(19) United States
(12) Patent Application Publication
(10) Pub. No.: US 2016/0045505 A1
(43) Pub. Date: Feb. 18, 2016

(54) COMPOUNDS AND COMPOSITIONS FOR THE TREATMENT OF PARASITIC DISEASES

(71) Applicants: Agnes VIDAL, San Diego, CA (US); Pranab Kumar MISHRA, West Bengal (IN)

(72) Inventors: Jan JIRICEK, Singapore (SG); Fang LIANG, Encinitas, CA (US); Casey Jacob Nelson MATHISON, San Diego, CA (US); Pranab Kumar MISHRA, Holland, PA (US); Valentina MOLTENI, San Diego, CA (US); Advait Suresh Nagle, San Diego, CA (US); Frantisek SUPEK, San Diego, CA (US); Liying Jocelyn TAN, Singapore (SG); Agnes VIDAL, Spring Valley, CA (US); Vince YEH, San Diego, CA (US)

(73) Assignee: NOVARTIS AG, Basel (CH)

(21) Appl. No.: 14/776,663

(22) PCT Filed: Mar. 13, 2014
(86) PCT No.: PCT/US2014/026445

§ 371 Date: Sep. 14, 2015

Related U.S. Application Data
(60) Provisional application No. 61/789,470, filed on Mar. 15, 2013.
(60) Provisional application No. 61/944,213, filed on Feb. 25, 2014.

(51) Int. Cl. A61K 31/519; C07D 519/00; A61K 31/695; A61K 31/553; C07F 7/08; C07D 487/04; A61K 31/5377

(52) U.S. Cl. CPC A61K 31/519; C07D 487/04; C07D 519/00; A61K 31/5377; A61K 31/553; C07F 7/0814; A61K 31/695

(57) ABSTRACT

The present invention provides compounds of formula (I): (Formula (I)) or a pharmaceutically acceptable salt, tautomer, or stereoisomer, thereof, wherein the variables are as defined herein. The present invention further provides pharmaceutical compositions comprising such compounds and methods of using such compounds for treating, preventing, inhibiting, ameliorating, or eradicating the pathology and/or symptomology of a disease caused by a parasite, such as Leishmaniasis, Human African Trypanosomiasis and Chagas disease.

### CROSS-REFERENCE TO RELATED APPLICATION

This application claims the benefit of priority to U.S. Provisional Patent Applications No. 61/789,470 filed 15 Mar., 2013 and No. 61/944,213 filed 25 Feb., 2014. The full disclosures of said applications are incorporated herein by reference in their entirety and for all purposes.

### BACKGROUND OF THE INVENTION

1. Field of the Invention

The invention provides a class of compounds, pharmaceutical compositions comprising such compounds and methods of using such compounds to treat or prevent Leishmaniasis.

2. Background

Leishmaniasis is a disease caused by protozoan parasites that belong to the genus Leishmania and is transmitted by the bite of certain species of sand fly.

Leishmaniasis is mostly a disease of the developing world, and is rarely known in the developed world outside a small number of cases, mostly in instances where troops are stationed away from their home countries. Leishmaniasis can be transmitted in many tropical and subtropical countries, and is found in parts of about 88 countries. Approximately 350 million people live in these areas. The settings in which leishmaniasis is found range from rainforests in Central and South America to deserts in West Asia and the Middle East. It affects as many as 12 million people worldwide, with 1.5-2 million new cases each year. The visceral form of leishmaniasis has an estimated incidence of 500,000 new cases and 60,000 deaths each year. More than 90 percent of the world's cases of visceral leishmaniasis are in India, Bangladesh, Nepal, Sudan, and Brazil. Kabul is estimated as the largest center of cutaneous leishmaniasis in the world, with approximately 67,500 cases as of 2004.

There are four main forms of Leishmaniasis. Cutaneous leishmaniasis is the most common form of leishmaniasis. Visceral leishmaniasis, also called kala-azar, is the most serious form in which the parasites migrate to the vital organs. Visceral leishmaniasis is caused by the parasite Leishmania donovani, and is potentially fatal if untreated.

Currently, no vaccines are in routine use.

The two main therapies for visceral leishmaniasis are the antimony derivatives sodium stibogluconate (Pentostam®) and meglumine antimoniate (Glucantim®). Sodium stibogluconate has been used for about 70 years and resistance to this drug is a growing problem. In addition, the treatment is relatively long and painful, and can cause undesirable side effects. Amphotericin (AmBisome) is now the treatment of choice. Miltefosine (Impavido), and paromomycin are the other treatment alternatives. These drugs are known to produce a definitive cure in >90% of patients. Amphotericin (AmBisome) is expansive and has to be given intravenously; it is not affordable to most patients affected. Paromomycin, although affordable, requires intramuscular injections for 3 weeks; compliance is a major issue. Miltefosine is an oral drug and has shown to be more effective and better tolerated than other drugs. However, there are problems associated with the use of miltefosine that arise from its teratogenicity and pharmacokinetics. Miltefosine was shown to be much slower eliminated from the body and was still detectable five months after the end of treatment. The presence of subtherapeutic miltefosine concentrations in the blood beyond five months after treatment might contribute to the selection of resistant parasites and, moreover, the measures for preventing the teratogenic risks of miltefosine must be reconsidered. This led to some reluctance to taking Miltefosine by affected populations.

The Drugs for Neglected Diseases Initiative is actively facilitating the search for novel therapeutics. Our invention meets that needs.

Human African trypanosomiasis (HAT), also known as African sleeping sickness, is a vector-borne parasitic disease caused by the protozoa Trypanosoma brucei. There are two subspecies that infect humans, T.b. gambiense and T.b. rhodesiense, with the former accounting for over 95% of reported cases and the latter accounting for the remaining reported cases. The parasites are transmitted to humans by tsetse fly (Glossina genus) bites which have acquired their infection from human beings or from animals harboring the human pathogenic parasites.

The disease has been recorded as occurring in 36 countries, all in subtropical and equatorial Africa. It is endemic in southeast Uganda and western Kenya. In 1995, the WHO estimated that 300,000 people were afflicted with the disease. In its 2001 report, the WHO set the figure of people at risk of infection at 60 million, of which only 4 to 5 million had access to any kind of medical monitoring. In 2006, the WHO estimated that about 70,000 people could have the disease, and many cases are believed to go unreported. About 48,000 people died of sleeping sickness in 2008. Public health efforts in prevention and the eradication of the tsetse fly population have proven to be successful in controlling the spread of the disease; under 10,000 new cases were reported in 2009 according to WHO figures, which represents a huge decrease from the estimated 300,000 new cases in 1998.

African trypanosomiasis symptoms occur in two stages. In the first stage, known as the haemolymphatic phase, the trypanosomes multiply in subcutaneous tissues, blood and lymph. The haemolymphatic phase is characterized by bouts of fever, headaches, Joint pains and itching. In the second stage, the neurological phase, the parasites cross the blood-brain barrier to infect the central nervous system. It is in this stage when more obvious signs and symptoms of the disease appear: changes of behaviour, confusion, sensory disturbances and poor coordination. Disturbance of the sleep cycle, which gives the disease its name, is an important feature of the second stage of the disease. Without treatment, the disease is invariably fatal, with progressive mental deterioration leading to coma, systemic organ failure, and death.

Four drugs are registered for the treatment of sleeping sickness. The protocol depends on the stage of the disease. The current standard treatment for first-stage disease is intravenous or intramuscular pentamidine (for T.b. gambiense), or intravenous suramin (for T.b. rhodesiense). The current standard treatment for second-stage disease is: Intravenous melarsoprol, or interavenous melarsoprol in combination with oral nifurtimox, intravenous eflornithine only or eflornithine in combination with nifurtimox. All of the drugs have undesirable or sometime serious side effects. For example, 3-10% of patients those injected with Melarsoprol (Arsobal), an organoarsenical, developed reactive encephalopathy (convulsions, progressive coma, or psychotic reactions), and 10-70% of such cases result in death.

Chagas disease, also called American trypanosomiasis, is a tropical parasitic disease caused by the flagellate protozoan Trypanosoma cruzi. T. cruzi is commonly transmitted to humans and other mammals by the blood-sucking “kissing bugs” of the subfamily Triatominae (family Reduviidae).

Chagas disease is contracted primarily in the Americas. It is endemic in twenty one Central and Latin American countries; particularly in poor, rural areas of Mexico, Central America, and South America. Large-scale population movements from rural to urban areas of Latin America and to other regions of the world have increased the geographic distribution of Chagas disease, and cases have been noted in many countries, particularly in Europe. Although there are triatomine bugs in the U.S., only very rarely vector borne cases of Chagas disease have been documented.

Each year, an estimated 10 to 15 million people across the world are infected with Chagas disease, most of whom do not know they are infected. Every year, 14,000 people die as a consequence of the disease. In Central and South America, Chagas kills more people than any other parasite-borne disease, including malaria. By applying published seroprevalence figures to immigrant populations, CDC estimates that more than 300,000 persons with Trypanosoma cruzi infection live in the United States. Most people with Chagas disease in the United States acquired their infections in endemic countries.

Chagas disease has an acute and a chronic phase. If untreated, infection is lifelong. Acute Chagas disease occurs immediately after infection, may last up to a few weeks or months, and parasites may be found in the circulating blood. Infection may be mild or asymptomatic. There may be fever or swelling around the site of inoculation (where the parasite entered into the skin or mucous membrane). Rarely, acute infection may result in severe inflammation of the heart muscle or the brain and lining around the brain. The initial acute phase is responsive to antiparasitic treatments, with 60-90% cure rates. Following the acute phase, most infected people enter into a prolonged asymptomatic form of disease (called “chronic indeterminate”) during which few or no parasites are found in the blood. During this time, most people are unaware of their infection. Many people may remain asymptomatic for life and never develop Chagas-related symptoms. However, an estimated 20-30% of infected people will develop debilitating and sometimes life-threatening medical problems over the course of their lives.

The symptoms of Chagas disease vary over the course of an infection. In the early, acute stage, symptoms are mild and usually produce no more than local swelling at the site of infection. The initial acute phase is responsive to antiparasitic treatments, with 60-90% cure rates. After 4-8 weeks, individuals with active infections enter the chronic phase of Chagas disease that is asymptomatic for 60-80% of chronically infected individuals through their lifetime.

There is no vaccine against Chagas disease. Treatment for Chagas disease focuses on killing the parasite and managing signs and symptoms.

During the acute phase of Chagas disease, the drugs currently available for treatment are benznidazole and nifurtimox. Once Chagas disease reaches the chronic phase, medications aren't effective for curing the disease. Instead, treatment depends on the specific signs and symptoms. However, problems with these current therapies include their diverse side effects, the length of treatment, and the requirement for medical supervision during treatment. Resistance to the two frontline drugs has already occurred. The antifungal agent Amphotericin b has been proposed as a second-line drug, but this drug is costly and relatively toxic.

In view of the foregoing, it is desirable to develop novel compounds as antiparasitic agents.

### SUMMARY OF THE INVENTION

The invention is related to compounds of Formula A:

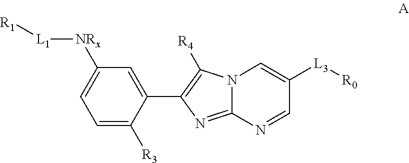

or a pharmaceutically acceptable salt, or stereoisomer thereof; wherein
Rₓ is hydrogen or C₁₋₄alkyl;
L₁ is a bond, —CH₂C(O)—, —C(O)— or —S(O)₂—;
R₁ is selected from hydrogen, nitro, C₁₋₄alkyl, C₂₋₄alkenyl, C₁₋₆alkoxy, —NR₇ₐR_{7b}, C₃₋₆cycloalkyl, C₄₋₇heterocycloalkyl, C₅₋₆heterocycloalkenyl, phenyl, benzyl and C₅₋₉heteroaryl; or —NRₓL₁R₁ is nitro; or R₁ and Rₓ, with L₁ and N to which R₁ and Rₓ are respectively attached to form a C₄₋₉heterocyclyl optionally substituted by 1 or 2 oxo, wherein
   R₇ₐ is hydrogen or C₁₋₄alkyl;
   R_{7b} is selected from hydrogen, C₁₋₄alkyl, C₁₋₄haloalkyl, C₁₋₄alkoxy, amino, C₃₋₆cycloalkyl, C₁₋₄alkoxy substituted C₃₋₆cycloalkyl, and C₅₋₆heteroaryl, wherein the C₅₋₆heteroaryl is optionally substituted by 1 to 2 substituents independently selected from halo, C₁₋₄alkyl, C₁₋₄alkoxyl and amino; and wherein the available hydrogens of the C₁₋₄alkyl of R₇ₐ and R_{7b} are each optionally replaced by deuterium;
   the C₁₋₄alkyl, C₂₋₄alkenyl, C₁₋₆alkoxy, amino, C₃₋₆cycloalkyl, C₄₋₇heterocycloalkyl, C₅₋₆heterocycloalkenyl, phenyl, or C₅₋₉heteroaryl of R₁ is optionally substituted by 1 to 4 substituents independently selected from hydroxy, halo, cyano, oxo, C₁₋₄alkyl, C₁₋₄hydroxyalkyl, C₁₋₄haloalkyl, C₁₋₆alkoxy, C₁₋₄haloalkoxy, C₁₋₄alkylcarbonyloxy, hydroxycarbonyl, C₁₋₄alkylcarbonyl, C₁₋₄alkoxycarbonyl, aminocarbonyl, C₁₋₄alkylsulfonyl, amino, C₁₋₄alkylamino, C₁₋₄alkylcarbonylamino, C₁₋₄alkylcarbonyl-N—C₁₋₄alkylamino, C₁₋₆alkoxycarbonylamino, C₁₋₄alkoxycarbonyl-N—C₁₋₄alkylamino, C₁₋₆alkoxy substituted C₃₋₆cycloalkyl, C₃₋₆cycloalkyl, C₄₋₆heterocycloalkyl, phenyl, and C₅₋₆heteroaryl; wherein the C₃₋₆cycloalkyl, C₄₋₆heterocycloalkyl, C₅₋₆heterocycloalkyl, phenyl or C₅₋₆heteroaryl substituent is optionally substituted by 1 to 2 substituent independently selected from halo, C₁₋₄alkyl, C₁₋₄alkylamino, or C₄₋₆heterocycloalkyl;
R₃ is selected from hydrogen, halo, cyano, C₁₋₄alkyl and C₁₋₄haloalkyl;
R₄ is selected from hydrogen, halo, C₁₋₄alkyl, C₁₋₄haloalkyl, and —C(O)R₁₀, wherein R₁₀ is selected from hydroxy, C₁₋₄alkyl, C₁₋₄alkoxy, amino, C₁₋₄alkylamino, C₃₋₆cycloalkyl and C₄₋₆heterocycloalkyl, wherein the C₁₋₄alkyl, C₁₋₄alkoxy, amino, C₁₋₄alkylamino, C₃₋₆cycloalkyl or C₄₋₆heterocycloalkyl of R₁₀ is optionally substituted by 1 to 2 substituents independently selected from hydroxyl, halo and C₁₋₄alkyl;
L₃ is selected from a bond, C₃₋₇cycloalkyl, C₄₋₇heterocycloalkyl, C₄₋₇cycloalkenyl, C₅₋₇heterocycloalkenyl, phenyl, and C₅₋₆heteroaryl;
R₀ is selected from hydrogen, hydroxy, halo, oxo, nitro, —N═CHN(CH₃)₂, C₁₋₆alkyl, C₁₋₄haloalkyl, C₂₋₄alkenyl, C₂₋₄alkynyl, C₁₋₄alkoxy, C₄₋₆heterocycloalkyloxy, —C(O)R₆, —NR₂ₐR_{2b}, —NR₅C(O)R₆, —NR₅S(O)₂R₈, —S(O)₂R₈, tri-C₁₋₄alkylsilyl, C₃₋₆cycloalkyl, C₄₋₆heterocycloalkyl, C₃₋₆cycloalkenyl, C₄₋₆heterocycloalkenyl, phenyl, and C₅₋₆heteroaryl; provided when L₃ is a bond, R₀ is not hydrogen; wherein
   the C₁₋₆alkyl, C₂₋₄alkenyl, C₂₋₄alkynyl, or C₁₋₄alkoxy of R₀ is optionally substituted by 1-2-substituents independently selected from hydroxy, halo, C₁₋₄alkoxy, trimethylsilylC₁₋₄alkoxy, C₁₋₄alkoxycarbonyl, —NR₁₁ₐR_{11b}, C₁₋₄alkoxycarbonylamino, C₅₋₆heterocycloalkyloxy, C₃₋₇cycloalkyl, C₅₋₆heterocycloalkyl, phenyl and C₅₋₆heteroaryl, wherein the C₃₋₇cycloalkyl, C₅₋₆heterocycloalkyl, phenyl or C₅₋₆heteroaryl substituent is optionally substituted by hydroxy, halo, C₁₋₄alkyl or C₁₋₄alkoxycarbonylamino, and wherein R₁₁ₐ and R_{11b} are independently hydrogen or C₁₋₄alkyl;
   the C₄₋₆heterocycloalkyloxy, C₃₋₆cycloalkyl, C₄₋₆heterocycloalkyl, C₄₋₆heterocycloalkenyl, phenyl or C₅₋₆heteroaryl of R₀ is optionally substituted by 1 to 4 substituents independently selected from hydroxy, halo, oxo, C₁₋₄alkyl, C₁₋₄hydroxyalkyl, C₁₋₄haloalkyl, —(CH₂)₀₋₄NRₐR_{b}, C₅₋₆arylC₁₋₄alkyl, C₄₋₆heterocycloalkylC₁₋₄alkyl, C₁₋₄alkoxy, C₁₋₄alkoxycarbonyl, C₁₋₄alkylsulfonyl, C₄₋₆heterocycloalkyl, wherein Rₐ and R_{b} are each independently hydrogen, C₁₋₄alkyl or C₃₋₆cycloalkyl;
   R₂ₐ is selected from hydrogen, C₁₋₄alkyl, C₁₋₄alkoxyC₁₋₄alkyl, C₁₋₄alkylcarbonyloxyC₁₋₄alkyl, and a C₁₋₄haloalkyl substituted C₅₋₆heteroaryl;
   R_{2b} is selected from hydrogen, C₁₋₆alkyl, C₁₋₆haloalkyl, aminoC₁₋₄alkyl, C₁₋₄alkoxy, C₁₋₄alkoxycarbonyl, C₃₋₆cycloalkyl, C₄₋₆heterocycloalkyl, C₅₋₆heterocycloalkenyl, and C₅₋₆heteroaryl, wherein
      the C₁₋₆alkyl of R_{2b} is optionally substituted by C₁₋₄alkoxy, amino, C₁₋₄alkylamino, C₄₋₆cycloalkyl, C₄₋₆heterocycloalkyl, phenyl or C₅₋₆heteroaryl, wherein the C₄₋₆cycloalkyl, C₄₋₆heterocycloalkyl, phenyl or C₅₋₆heteroaryl substituent is optionally substituted by hydroxy, halo, C₁₋₄alkyl, C₁₋₄haloalkyl, or C₁₋₄alkyoxycarbonylamino;
      the C₄₋₆heterocycloalkyl, C₅₋₆heterocycloalkenyl or C₅₋₆heteroaryl of R_{2b} is optionally substituted by 1 to 2 substituents independently selected from C₁₋₄alkyl and C₁₋₄haloalkyl;
   R₅ is hydrogen or C₁₋₄alkyl;
   R₆ is selected from hydrogen, C₁₋₆alkyl, C₁₋₆hydroxyalkyl, C₁₋₆haloalkyl, C₂₋₆alkenyl, C₁₋₄alkoxy, C₁₋₆haloalkoxy, C₁₋₄cycloalkoxy, C₂₋₆alkenyloxy, C₃₋₆cycloalkyloxy, benzyloxy, —NR₁₂ₐR_{12b}, C₃₋₇cycloalkyl, C₄₋₇heterocycloalkyl, C₅₋₇cycloalkenyl, C₅₋₆heterocycloalkenyl, phenyl, and C₅₋₆heteroaryl, wherein
      R₁₂ₐ is hydrogen or C₁₋₆alkyl,
      R_{12b} is selected from hydrogen, C₁₋₆alkyl, amino and C₁₋₆alkylamino;
      the C₁₋₆alkyl, C₂₋₆alkenyl, C₁₋₄alkoxy, C₃₋₆cycloalkyloxy, or amino of R₆ is optionally substituted by 1 to 2 substituents independently selected from hydroxy, halo, C₁₋₄alkyl, C₁₋₄haloalkyl, C₁₋₄alkoxy, C₁₋₄haloalkoxy, C₁₋₄alkoxyC₁₋₄alkoxy, —NR₉ₐR_{9b}, C₃₋₆cycloalkyl, C₄₋₇heterocycloalkyl, and C₅₋₆heteroaryl, wherein the C₅₋₆heterocycloalkyl or C₅₋₆heteroaryl substituent is optionally substituted by 1-2 substituents independently selected from hydroxyl, C₁₋₄alkyl and C₁₋₄alkoxycarbonyl, and wherein R₉ₐ is hydrogen or C₁₋₄alkyl and R_{9b} is selected from hydrogen, C₁₋₄alkyl, C₁₋₄alkylcarbonyl and C₁₋₄alkoxycarbonyl;
      the C₃₋₆cycloalkyloxy, C₃₋₇cycloalkyl, C₄₋₇heterocycloalkyl, C₅₋₇cycloalkenyl, or C₅₋₆heterocycloalkenyl of R₆ is optionally substituted by 1 to 3 substituents independently selected from hydroxy, halo, cyano, oxo, C₁₋₄alkyl, haloC₁₋₄alkyl, C₁₋₄alkoxylC₁₋₄alkyl, C₁₋₄alkoxycarbonylaminoC₁₋₄alkyl, C₁₋₄alkoxyl, C₁₋₄alkoxycarbonyl, amino, aminocarbonyl, and
      the phenyl or C₅₋₆heteroaryl of R₆ is optionally substituted with 1-2 substituents independently selected from hydroxy, halo, cyano, C₁₋₄alkyl, C₁₋₄hydroxyalkyl, C₁₋₄haloalkyl, C₁₋₄alkoxy, C₁₋₄alkoxycarbonyl, —(CH₂)₀₋₄NR₁₃ₐR_{13b}, and C₁₋₄alkoxycarbonylamino, wherein the C₁₋₄alkoxy substituent is optionally substituted by C₁₋₄alkylC₅₋₆heterocycloalkyl, and wherein R₁₃ₐ and R_{13b} are each independently hydrogen or C₁₋₄alkyl; and
   R₈ is C₁₋₄alkyl or C₁₋₄alkylamino.

In a second aspect, the present invention provides a pharmaceutical composition which contains a compound of the invention selected from Formula A, a subformulae thereof, an N-oxide derivative, individual isomers and mixture of isomers thereof; or a pharmaceutically acceptable salt thereof, in admixture with one or more suitable excipients.

In a third aspect, the present invention provides a method of treating a disease in an animal in which a compound of the invention can prevent, inhibit or ameliorate the pathology and/or symptomology of a disease caused by a parasite of the Leishmania genus, for example, Leishmania donovani, Leishmania infantum, Leishmania braziliensis, Leishmania panamensis, Leishmania guayanensis, Leishmania amazonensis, Leishmania mexicana, Leishmania tropica, Leishmania maJor, Trypanosoma cruzi, and Trypanosoma brucei and a parasite of the Trypanosoma genus, for example, Trypanosoma cruzi and Trypanosoma brucei, which method comprises administering to the animal a therapeutically effective amount of a compound selected from Formula A, and subformulae thereof, an N-oxide derivative, individual isomers and mixture of isomers thereof, or a pharmaceutically acceptable salt thereof.

In a fourth aspect, the present invention provides a compound of Formula A, a subformulae thereof, an N-oxide derivative, individual isomers and mixture of isomers thereof, or a pharmaceutically acceptable salt thereof, for treating, preventing, inhibiting, ameliorating, or eradicating the pathology and/or symptomology of a disease caused by a parasite of the Leishmania genus, for example, Leishmania donovani, Leishmania infantum, Leishmania braziliensis, Leishmania panamensis, Leishmania guayanensis, Leishmania amazonensis, Leishmania mexicana, Leishmania tropica, Leishmania maJor, Trypanosoma cruzi, and Trypanosoma brucei and a parasite of the Trypanosoma genus, such as, for example, Trypanosoma cruzi and Trypanosoma brucei. Particularly, the parasite is a Leishmania, and the disease is Leishmanaisis.

In a fifth aspect, the present invention provides the use of a compound selected from Formula A, a subformulae thereof, an N-oxide derivative, individual isomers and mixture of isomers thereof, or a pharmaceutically acceptable salt thereof, in the manufacture of a medicament for treating a disease caused by a parasite in an animal. The disease may be Leishmaniasis, Human African Trypanosomiasis and/or Chagas disease.

Unless specified otherwise, the term “compounds of the present invention” refers to compounds of Formula A, a subformulae thereof, salts of the compound, hydrates or solvates of the compounds, salts, as well as all stereoisomers (including diastereoisomers and enantiomers), tautomers and isotopically labeled compounds (including deuterium substitutions). Compounds of the present invention comprise polymorphs of compounds of Formula A, a subformulae thereof, and salts thereof.

### DETAILED DESCRIPTION OF THE INVENTION

### Definitions

For purposes of interpreting this specification, the following definitions will apply and whenever appropriate, terms used in the singular will also include the plural and vice versa.

“Acyl” as used herein refers to the radical —C(═O)Rₐ, where Rₐ is hydrogen or a non-hydrogen substituent on the carbonyl carbon, forming different carbonyl-containing groups including, but are not limited to, acids, acid halides, aldehydes, amides, esters, and ketones.

“Alkoxy” as used herein refers the radical —O-alkyl, wherein the alkyl is as defined herein. C_{X}alkoxy and C_{X-Y}alkoxy as used herein describe alkoxy groups where X and Y indicate the number of carbon atoms in the alkyl chain. Representative examples of C₁₋₁₀alkoxy include, but are not limited to, methoxy, ethoxy, propoxy, 2-propoxy, butoxy, tert-butoxy, pentyloxy, hexyloxy, heptyloxy, octyloxy and decyloxy. The alkyl portion of the alkoxy may be optionally substituted, and the substituents include those described for the alkyl group below.

“Alkyl” as used herein refers to a fully saturated branched or unbranched hydrocarbon chain having up to 10 carbon atoms. C_{X} alkyl and C_{X-Y} alkyl as used herein describe alkyl groups where X and Y indicate the number of carbon atoms in the alkyl chain. For example, C₁₋₁₀ alkyl refers to an alkyl radical as defined above containing one to ten carbon atoms. C₁₋₁₀ alkyl includes, but are not limited to, methyl, ethyl, n-propyl, isopropyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. Alkyl represented along with another radical like arylalkyl, heteroarylalkyl, alkoxyalkyl, alkoxyalkyl, alkylamino, where the alkyl portion shall have the same meaning as described for alkyl and is bonded to the other radical. For example, (C₆₋₁₀)aryl(C₁₋₃)alkyl includes, benzyl, phenylethyl, 1-phenylethyl, 3-phenylpropyl, 2-thienylm ethyl, 2-pyridinylmethyl and the like.

Unless stated otherwise specifically in the specification, an alkyl group may be unsubstituted or substituted by one or more substituents to the extent that such substitution makes sense chemically. Typical substituents include, but are not limited to halo, hydroxyl, alkoxy, cyano, amino, acyl, aryl, arylalkyl, and cycloalkyl, or an heteroforms of one of these groups, and each of which can be substituted by the substituents that are appropriate for the particular group.

“Alkenyl” as used herein refers to a straight or branched, hydrocarbon chain having up to 10 carbon atoms and at least one carbon-carbon double bond. C_{X}alkenyl and C_{X-Y}alkenyl as used herein describe alkenyl groups where X and Y indicate the number of carbon atoms in the alkenyl chain. Examples of C₂₋₇alkenyl include vinyl, allyl, isopropenyl, pentenyl, hexenyl, heptenyl, 1-propenyl, 2-butenyl, 2-methyl-2-butenyl, and the like. The alkenyl may be optionally substituted, and the substituents include those described for the alkyl group descried herein.

“Alkynyl” as used herein refers to a straight or branched, hydrocarbon chain having up to 10 carbon atoms and at least one carbon-carbon triple bond. C_{X}alkenyl and C_{X-Y}alkenyl as used herein describe alkynyl groups, where X and Y indicate the number of carbon atoms in the alkynyl chain. For example, C₂₋₇alkenyl include, but are not limited to, ethynyl, propargyl, 3-methyl-1-pentynyl, 2-heptynyl and the like. An alkynyl may be optionally substituted, and the substituents include those described for the alkyl group described herein.

“Alkylene” as used herein refers to a divalent alkyl group defined herein. Examples of C₁₋₁₀alkylene includes, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene. An alkylene group may be optionally substituted, and the substituents include those described for the alkyl group described herein.

“Alkenylene” as used herein refers to a divalent alkenyl group defined herein. Examples of C₁₋₃alkenylene include, but are not limited to, ethene-1,2-diyl, propene-1,3-diyl, and methylene-1,1-diyl. An alkenylene may be optionally substituted, and the substituents include those described for the alkyl group described herein.

“Alkynylene” as used herein refers to a divalent alkynyl group defined herein. Examples of alkynylene include ethyne-1,2-diylene, propyne-1,3-diylene, and the like. An alkynylene may be optionally substituted, and the substituents include those described for the alkyl group described herein.

“Amino” as used herein refers to the radical —NH₂. When an amino is described as “substituted” or “optionally substituted”, the term includes NR′R″ wherein each R′ and R″ is independently H, or is an alkyl, alkenyl, alkynyl, acyl, aryl, aryl, cycloalkyl, arylalkyl cycloalkylalkyl group or a heteroform of one of these groups, and each of the alkyl, alkenyl, alkynyl, acyl, aryl, arylalkyl or groups or heteroforms of one of these groups, each of which is optionally substituted with the substituents described herein as suitable for the corresponding group.

The term “amino” also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Alkylamino” as used herein refers to the radical —NRₐR_{b}, where at least one of, or both, Rₐ and R_{b} are an alkyl group as described herein. A C₁₋₄alkylamino group includes —NHC₁₋₄alkyl and —N(C₁₋₄alkyl)₂; e.g., —NHCH₃, —N(CH₃)₂, —NH(CH₂CH₃), —N(CH₂CH₃)₂, and the like.

“Aromatic” as used herein refers to a moiety wherein the constituent atoms make up an unsaturated ring system, where all atoms in the ring system are sp² hybridized and the total number of pi electrons is equal to 4n+2. An aromatic ring may be such that the ring atoms are only carbon atoms or may include carbon and non-carbon atoms (see Heteroaryl).

“Aryl” as used herein refers to a 6-14 membered monocyclic or polycyclic aromatic ring assembly where all the ring atoms are carbon atoms. Typically, the aryl is a 6 membered monocyclic, a 10-12 membered bicyclic or a 14-membered fused tricyclic aromatic ring system. C_{X}aryl and C_{X-Y}aryl as used herein describe an aryl group where X and Y indicate the number of carbon atoms in the ring system. C₆₋₁₄aryls include, but are not limited to, phenyl, biphenyl, naphthyl, azulenyl, and anthracenyl.

An aryl may be unsubstituted or substituted by 1-5 (such as one, or two, or three) substituents independently selected from the group consisting of hydroxy, thiol, cyano, nitro, C₁₋₄alkyl, C₁₋₄alkenyl, C₁₋₄alkynyl, C₁₋₄alkoxy, thioC₁₋₄alkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄alkyl)amino, sulfonyl, sulfamoyl, alkylsulfamoyl, C₁₋₄alkylaminosulfonyl, aryl, heteroaryl, cycloalkyl and heterocycloalkyl, wherein each of the afore-mentioned substitutents may be further substituted by one or more substituents independently selected from halogen, alkyl, hydroxyl or C₁₋₄alkoxy groups.

When an “aryl” is represented along with another radical like “arylalkyl”, “aryloxyalkyl”, “aryloxycarbonyl”, “aryloxy-carbonylalkyl”, the aryl portion shall have the same meaning as described in the above-mentioned definition of “aryl”.

“Aryloxy” as used herein, refers to the radical —O-aryl, wherein aryl is as defined herein.

“Bicyclic” or “bicyclyl” as used here in refers to a ring assembly of two rings where the two rings are fused together, linked by a single bond or linked by two bridging atoms. The rings may be a carbocyclyl, a heterocyclyl, or a mixture thereof.

“Bridging ring” as used herein refers to a polycyclic ring system where two ring atoms that are common to two rings are not directly bound to each other. One or more rings of the ring system may also comprise heteroatoms as ring atoms. Non-exclusive examples of bridging rings include norbornanyl, 7-oxabicyclo[2.2.1]heptanyl, adamantanyl, and the like.

“Carbamoyl” as used herein refers to the radical —C(O)NRₐ- where Rₐ is H, or is an alkyl, alkenyl, alkynyl, acyl, aryl, or arylalkyl group or a heteroform of one of these groups, and each of the alkyl, alkenyl, alkynyl, acyl, aryl, arylalkyl or heteroforms of one of these groups is optionally substituted with the substituents described herein as suitable for the corresponding group.

“Carbamate” as used herein refers to the radical —OC(O)NRₐR_{b} where Rₐ and R_{b} are each independently H, or is an alkyl, alkenyl, alkynyl, acyl, aryl, or arylalkyl group or a heteroform of one of these groups, and each of the alkyl, alkenyl, alkynyl, acyl, aryl, arylalkyl or heteroforms of one of these groups is optionally substituted with the substituents described herein as suitable for the corresponding group.

“Cycloalkyl”, as used herein, means a radical comprising a non-aromatic, saturated or partially unsaturated, monocyclic, bicyclic, tricyclic, fused, bridged or spiro polycyclic hydrocarbon ring system of 3-20 carbon atoms. C_{X}cycloalkyl and C_{X-Y}cycloalkyl are typically used where X and Y indicate the number of carbon atoms in the ring assembly. For example, C₃₋₆cycloalkyl includes cyclopropyl, cyclobutyl, cyclopentyl, cyclohexyl, cyclohexenyl, 2,5-cyclohexadienyl.

Exemplary monocyclic cycloalkyl include, but are not limited to, cyclopropyl, cyclobutyl, cyclopentyl, cyclopentenyl, cyclohexyl and cyclohexenyl and the like.

Exemplary bicyclic cycloalkyls include bornyl, norbornanyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl. Exemplary tricyclic cycloalkyl groups include, for example, adamantyl.

A cycloalkyl may be unsubstituted or substituted by one, or two, or three, or more substituents independently selected from the group consisting of hydroxyl, thiol, cyano, nitro, oxo, alkylimino, C₁₋₄alkyl, C₁₋₄alkenyl, C₁₋₄alkynyl, C₁₋₄alkoxy, C₁₋₄thioalkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄alkyl)amino, sulfonyl, sulfamoyl, alkylsulfamoyl, C₁₋₄alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁₋₄alkoxy groups.

“Cycloalkylene”, as used herein, refers to a divalent radical comprising a cycloalkyl ring assembly as defined herein.

“Cycloalkoxy”, as used herein, refers to —O-cycloalkyl, wherein the cycloalkyl is defined herein. Representative examples of C₃₋₁₂cycloalklyoxy include, but are not limited to, monocyclic groups such as cyclopropoxy, cyclobutoxy, cyclopentyloxy, cyclopentenyloxy, cyclohexyloxy and cyclohexenyloxy and the like. Exemplary bicyclic hydrocarbon groups include bornyloxy, indyloxy, hexahydroindyloxy, tetrahydronaphthyloxy, decahydronaphthyloxy, bicyclo[2.1.1]hexyloxy, bicyclo[2.2.1]heptyloxy, bicyclo[2.2.1]heptenyloxy, 6,6-dimethylbicyclo[3.1.1]heptyloxy, 2,6,6-trimethylbicyclo[3.1.1]heptyloxy, bicyclo[2.2.2]octyloxy and the like. Exemplary tricyclic hydrocarbon groups include, for example, adamantyloxy.

“Cyano”, as used herein, refers to the radical —CN.

“EC₅₀”, refers to the molar concentration of an inhibitor or modulator that produces 50% efficacy.

“Fused ring”, as used herein, refers to a multi-ring assembly wherein the rings comprising the ring assembly are so linked that the ring atoms that are common to two rings are directly bound to each other. The fused ring assemblies may be saturated, partially saturated, aromatics, carbocyclics, heterocyclics, and the like. Non-exclusive examples of common fused rings include decalin, naphthalene, anthracene, phenanthrene, indole, benzofuran, purine, quinoline, and the like.

“Halo” or “halogen” as used herein refers to fluoro, chloro, bromo, and iodo.

“Haloalkyl”, or halo-substituted-alkyl” as used herein, refers to an alkyl as defined herein, which is substituted by one or more halo atoms defined herein. The haloalkyl can be mono-haloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. C_{X}haloalkyl and C_{X-Y}haloalkyl are typically used where X and Y indicate the number of carbon atoms in the alkyl chain. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A C₁₋₄perhaloalkyl group refers to a C₁₋₄alkyl group having all hydrogen atoms replaced with halo atoms.

“Haloalkoxy”, or halo-substituted-alkoxy” as used herein, refers to an alkoxy as defined herein, which is substituted by one or more halo atoms defined herein. The haloalkoxy can be mono-haloalkoxy, dihaloalkoxy or polyhaloalkoxy including perhaloalkoxy. A monohaloalkoxy can have one iodo, bromo, chloro or fluoro within the alkoxy group. Dihaloalkoxy and polyhaloalkoxy groups can have two or more of the same halo atoms or a combination of different halo groups within the alkoxy. C_{X}haloalkoxy and C_{X-Y}haloalkoxy are typically used where X and Y indicate the number of carbon atoms in the alkoxy chain. Non-limiting examples of C₁₋₄haloalkoxy include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, difluoroethoxy, difluoropropoxy, dichloroethoxy and dichloropropoxy. A C₁₋₄perhaloalkoxy group refers to a C₁₋₄alkoxy group having all hydrogen atoms replaced with halo atoms.

“Heteroalicyclic” as used herein refers to 3-14 membered, monocyclic or polycyclic, non-aromatic ring assembly including 1 to 4 heteroatoms as ring atom. The ring assembly may be saturated or partially unsaturated with one, two or more double or triple bonds. The heteroatoms may include nitrogen, oxygen and sulfur; the nitrogen atoms can be optionally quaternerized or oxidized and the sulfur atoms can be optionally oxidized. C_{X}heteroalicyclyl and C_{X-Y}heteroalicyclyl as used herein describe the heterocyclic where X and Y indicate the number of ring atoms in the ring assembly.

“Heteroaryl”, as used herein, refers to a 5-14 membered ring assembly (e.g., a 5-7 membered monocycle, an 8-10 membered bicycle, or a 13-14 membered tricyclic ring system) having 1 to 8 heteroatoms selected from N, O and S as ring atoms and the remaining ring atoms are carbon atoms. The nitrogen atoms of such heteroaryl rings can be optionally quaternerized and the sulfur atoms of such heteroaryl rings can be optionally oxidized. C_{X}heteroaryl and C_{X-Y}heteroaryl as used herein describe heteroaryls where X and Y indicate the number of ring atoms in the heteroaryl ring. Typical C₅₋₇heteroaryl groups include thienyl, furanyl, imidazolyl, pyrazolyl, pyrrolyl, pyrrolinyl, thiazolyl, 1,3,4-thiadiazolyl, isothiazolyl, oxazolyl, oxadiazole isoxazolyl, triazolyl, tetrazolyl, pyridyl, pyridazinyl, pyrazinyl, pyrazinyl, pyrimidinyl, and the like. Bicyclic or tricyclic C₈₋₁₄heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, quinazolinyle, pteridinyl, indolizine, imidazo[1,2a]pyridine, quinoline, quinolinyl, isoquinoline, phthalazine, quinoxaline, naphthyridine, naphthyridinyl, quinolizine, indolyl, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, purinyl, carbazole, acridine, phenazine, phenothiazene, phenoxazine, 1,2-dihydropyrrolo[3,2,1-hi]indole, indolizine, pyrido[1,2-a]indole and 2(1H)-pyridinone.

A heteroaryl may be unsubstituted or substituted with one or more substituents independently selected from hydroxyl, thiol, cyano, nitro, C₁₋₄alkyl, C₁₋₄alkenyl, C₁₋₄alkynyl, C₁₋₄alkoxy, thioC₁₋₄alkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄alkyl)amino, sulfonyl, sulfamoyl, alkylsulfamoyl, C₁₋₄alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁₋₄alkoxy groups.

When a heteroaryl is represented along with another radical like “heteroaryloxy”, “heteroaryloxyalkyl”, “heteroaryloxycarbonyl”, the heteroaryl portion shall have the same meaning as described in the above-mentioned definition of “heteroaryl”.

“Heteroaryloxy”, as used herein, refers to an —O-heteroaryl group, wherein the heteroaryl is as defined in this Application.

“Heteroatom”, as used herein, refers to an atom that is not a carbon atom. Particular examples of heteroatoms include, but are not limited to nitrogen, oxygen, and sulfur.

“Heterocycloalkyl”, as used herein, refers to a 4-20 membered, non-aromatic, saturated or partially unsaturated, monocyclic or polycyclic ring system, comprising 1-8 heteroatoms as ring atoms and that the remaining ring atoms are carbon atoms. The heteroatoms are selected from N, O, and S, preferably O and N. The nitrogen atoms of the heterocycloalkyl can be optionally quaternerized and the sulfur atoms of the heterocycloalkyl can be optionally oxidized. The heterocycloalkyl can include fused or bridged rings as well as spirocyclic rings. C_{X}heterocycloalkyl and C_{X-Y}heterocycloalkyl are typically used where X and Y indicate the number of ring atoms in the ring. Typically, the heterocycloalkyl is 4-8-membered monocyclic ring containing 1 to 3 heteroatoms, a 7 to 12-membered bicyclic ring system containing 1-5 heteroatoms, or a 10-15-membered tricyclic ring system containing 1 to 7 heteroatoms. Examples of C₄₋₆heterocycloalkyl include azetidinyl, tetrahydrofuran (THF), dihydrofuran, 1,4-dioxane, morpholine, 1,4-dithiane, piperazine, piperidine, 1,3-dioxolane, imidazolidine, imidazoline, pyrazolidinyl, pyrroline, pyrrolidine, tetrahydropyran, dihydropyran, oxathiolane, dithiolane, 1,3-dioxane, 1,3-dithiane, oxathiane, thiomorpholine, and the like

A heterocycloalkyl may be unsubstituted or substituted with 1-5 substituents (such as one, or two, or three) each independently selected from hydroxyl, thiol, cyano, nitro, oxo, alkylimino, C₁₋₄alkyl, C₁₋₄alkenyl, C₁₋₄alkynyl, C₁₋₄alkoxy, C₁₋₄thioalkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄alkyl)amino, sulfonyl, sulfamoyl, alkylsulfamoyl, C₁₋₄alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁₋₄alkoxy groups.

When a heterocycloalkyl forms part of other groups like “heterocycloalkyl-alkyl”, “heterocycloalkoxy”, “heterocycloalkyl-aryl”, the heteroaryl portion shall have the same meaning as described in the above-mentioned definition of “heteroaryl”

“Heterocycloalkylene”, as used herein, refers to a cycloalkylene, as defined in this Application, provided that one or more of the ring member carbon atoms is replaced by a heteroatom.

“Heterocycloalkyl fused to a phenyl” as used herein, refers to a bicyclic fused ring system that one of the rings is heterocycloalkyl as defined above and the other ring is a phenyl. A heterocycloalkyl fused to a phenyl includes but are not limited to benzo[b][1,4]oxazinyl, oxo-benzo[b][1,4]oxazinyl, tetrahydroquinoxalinyl, tetrahydroquinolinyl, indolinyl, benzo[d]imidazolyl, and the like.

“Heterocyclyl”, “heterocycle” or “heterocyclo”, as used herein, refers to a 3-20 membered, monocyclic or polycyclic ring system containing at least one heteroatom moiety selected from the group consisting of N, O, SO, SO₂, (C═O), and S, and preferably N, O, S, optionally containing one to four additional heteroatoms in each ring. C_{X}heterocyclyl and C_{X-Y}heterocyclyl are typically used where X and Y indicate the number of ring atoms in the ring system. Unless otherwise specified, a heterocyclyl may be saturated, partially unsaturated, aromatic or partially aromatic.

Hydroxy, as used herein, refers to the radical —OH.

“Hydroxyalkyl” or “hydroxyl-substituted alkyl” as used herein, refers to an alkyl as defined herein, having one or more of the available hydrogen of the alkyl replaced by a hydroxyl group. For example, a hydroxyC₁₋₄alkyl includes, but are not limited to, —CH₂CH₂OH, —CH(OH)CH₂CH₂OH, —CH(OH)CH₂CH(OH)CH₃.

“Hydroxyalkoxy” or “hydroxyl-substituted alkoxy” as used herein, refers to an alkoxy as defined herein, having one or more of the available hydrogen on the alkyl replaced by a hydroxyl group. For example, a hydroxyC₁₋₄alkoxy includes, but are not limited to, —OCH₂CH₂OH, —OCH(OH)CH₂CH₂OH, —OCH(OH)CH₂CH(OH)CH₃.

“Nitro”, as used herein, refers to the radical —NO₂.

“Oxo”, as used herein, refers to the divalent radical ═O

“Protected derivatives” means derivatives of inhibitors in which a reactive site or sites are blocked with protecting groups. Protected derivatives are useful in the preparation of inhibitors or in themselves may be active as inhibitors. Examples of protected group includes, but are not limited to, acetyl, tetrahydropyran, methoxymethyl ether, β-methoxyethoxym ethyl ether, ρ-methoxybenzyl, methylthiomethyl ether, pivaloyl, silyl ether, carbobenzyloxy, benzyl, tert-butoxycarbonyl, ρ-methoxyphenyl, 9-fluorenylm ethyloxycarbonyl, acetals, ketals, acylals, dithianes, methylesters, benzyl esters, tert-butyl esters, and silyl esters. A comprehensive list of suitable protecting groups can be found in T. W. Greene, Protecting Groups in Organic Synthesis, 3rd edition, John Wiley & Sons, Inc. 1999.

“Unsubstituted or substituted” or “optionally substituted” as used herein indicate the substituent bound on the available valance of a named group or radical. “Unsubstituted” as used herein indicates that the named group or radical will have no further non-hydrogen substituents. “Substituted” or “optionally substituted” as used herein indicates that at least one of the available hydrogen atoms of named group or radical has been (or may be) replaced by a non-hydrogen substituent.

Unless otherwise specified, examples of substituents may include, but are not limited to, halo, nitro, cyano, thio, oxy, hydroxy, carbonyloxy, C₁₋₆alkoxy, C₆₋₁₀aryloxy, heteroC₅₋₁₀aryloxy, carbonyl, oxycarbonyl, aminocarbonyl, amino, C₁₋₆alkylamino, sulfonamido, imino, sulfonyl, sulfinyl, C₁₋₆alkyl, C₁₋₆haloalkyl, hydroxyC₁₋₆alkyl, carbonylC₁₋₆alkyl, thiocarbonylC₁₋₁₀alkyl, sulfonylC₁₋₆alkyl, sulfinylC₁₋₆alkyl, C₁₋₁₀azaalkyl, iminoC₁₋₆alkyl, C₃₋₁₂cycloalkylC₁₋₆alkyl, C₄₋₁₅heterocycloalkylC₁₋₆alkyl, C₆₋₁₀arylC₁₋₆alkyl, C₅₋₁₀heteroarylC₁₋₆alkyl, C₁₀₋₁₂bicycloarylC₁₋₆alkyl, C₉₋₁₂heterobicycloarylC₁₋₆alkyl, C₃₋₁₂cycloalkyl, C₄₋₁₂heterocycloalkyl, C₉₋₁₂bicycloalkyl, C₃₋₁₂heterobicycloalkyl, C₄₋₁₂aryl, heteroC₁₋₁₀aryl, C₉₋₁₂bicycloaryl and C₄₋₁₂heterobicycloaryl.

“Sulfonyl”, as used herein, means the radical —S(O)₂—. It is noted that the term “sulfonyl” when referring to a monovalent substituent can alternatively refer to a substituted sulfonyl group, —S(═O)₂R, where R is hydrogen or a non-hydrogen substituent on the sulfur atom forming different sulfonyl groups including sulfonic acids, sulfonamides, sulfonate esters, and sulfones.

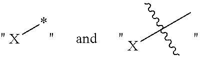

are symbols denoting the point of attachment of X, to other part of the molecule.

Any definition herein may be used in combination with any other definition to describe a composite structural group. By convention, the trailing element of any such definition is that which attaches to the parent moiety. For example, the composite group alkoxyalkyl would represent an alkoxy group attached to the parent molecule through an alkyl group.

It is noted in regard to all of the definitions provided herein that the definitions should be interpreted as being open ended in the sense that further substituents beyond those specified may be included. Hence, a C₁alkyl indicates that there is one carbon atom but does not indicate what are the substituents on the carbon atom. Hence, a C₁alkyl comprises methyl (i.e., —CH₃) as well as —CRₐR_{b}R_{c} where Rₐ, R_{b}, and R_{c} may each independently be hydrogen or any other substituent where the atom attached to the carbon is not a hydrogen atom. Hence, —CF₃, —CH₂OH and —CH₂CN, for example, are all C₁alkyls.

### DESCRIPTION OF THE PREFERRED EMBODIMENTS

The invention provides a novel class of compounds, pharmaceutical compositions comprising such compounds and methods of using such compounds to treat or prevent diseases or disorders associated with a parasite. In particular, the compounds can be used to treat leishmaniasis, Human Trypanosomiasis and/or Chagas disease. The compounds of the invention are effective in inhibiting, ameliorating, or eradicating the pathology and/or symptomology of the parasite.

In one embodiment, the compounds of the invention are of Formula A:

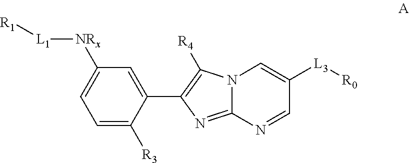

wherein the variables are as described in the Summary of the Invention.

In another embodiment of the compound of Formula A of the invention,
Rₓ is hydrogen or C₁₋₄alkyl;
L₁ is selected from a bond, —CH₂C(O)—, —C(O)— and —S(O)₂—;
R₁ is selected from hydrogen, C₁₋₄alkyl, C₂₋₄alkenyl, C₁₋₆alkoxy, C₁₋₆alkoxyC₁₋₆alkoxy, NR₇ₐR_{7b}, C₃₋₆cycloalkyl, C₄₋₇heterocycloalkyl, C₅₋₆heterocycloalkenyl, phenyl, benzyl, and C₅₋₉heteroaryl, or NRₓL₁R₁ is nitro; or R₁ and Rₓ, with L₁ and N to which R₁ and Rₓ are respectively attached are taken together to form a C₄₋₉heterocyclyl optionally substituted by 1 or 2 oxo, wherein
   R₇ₐ is hydrogen or C₁₋₄alkyl;
   R_{7b} is selected from hydrogen, C₁₋₄alkyl, C₁₋₄haloalkyl, C₁₋₆alkoxy, amino, C₃₋₆cycloalkyl, C₁₋₄alkoxy substituted C₃₋₆cycloalkyl, and C₅₋₆heteroaryl, wherein the C₅₋₆heteroaryl is optionally substituted by halo or C₁₋₄alkyl, and wherein the available hydrogens of the C₁₋₄alkyl of R₇ₐ or R_{7b} is optionally replaced by deuterium;
   the C₁₋₄alkyl or C₁₋₄alkenyl of R₁ is optionally substituted by 1 to 4 substituents each independently selected from C₁₋₄alkyl, —C₁₋₆alkoxy, C₁₋₄haloalkoxy, C₁₋₄alkylcarbonyloxy, amino, C₁₋₄alkylamino, C₃₋₆cycloalkyl, C₅₋₆heterocycloalkyl, phenyl and C₅₋₉heteroaryl,
   the C₃₋₆cycloalkyl, C₄₋₇heterocycloalkyl, C₅₋₆heterocycloalkenyl, phenyl or C₅₋₉heteroary of R₁ is optionally substituted by 1 to 4 substituents independently selected from hydroxy, halo, cyano, oxo, C₁₋₄alkyl, C₁₋₄hydroxyalkyl, C₁₋₄haloalkyl, C₁₋₆alkoxy, C₁₋₄haloalkoxy, hydroxycarbonyl, C₁₋₄alkylcarbonyl, C₁₋₄alkoxycarbonyl, aminocarbonyl, amino, C₁₋₄alkylamino, C₁₋₄alkylcarbonylamino, C₁₋₄alkylcarbonyl-N—C₁₋₄alkylamino, C₁₋₆alkoxycarbonylamino, C₁₋₄alkoxycarbonyl-N—C₁₋₄alkylamino, C₁₋₄alkylsulfonyl, C₃₋₆cycloalkyl, C₄₋₆heterocycloalkyl, pheny, and C₅₋₆heteroaryl, wherein the C₃₋₆cycloalkyl or C₄₋₆heterocycloalkyl substituent is optionally substituted by C₁₋₄alkyl;
R₃ is hydrogen or halo,
R₄ is hydrogen or halo;
L₃ is selected from a bond, C₃₋₇cycloalkyl, C₄₋₇heterocycloalkyl, C₄₋₇cycloalkenyl, C₅₋₇heterocycloalkenyl, phenyl, and C₅₋₆heteroaryl;
R₀ is selected from hydrogen, halo, oxo, C₁₋₆alkyl, C₁₋₄haloalkyl, C₂₋₄alkenyl, C₂₋₄alkynyl, C₁₋₄alkoxy, C₄₋₆heterocycloalkyloxy, —C(O)R₆, —NR₂ₐR_{2b}, NR₅C(O) R₆, NR₅S(O)₂R₈—, —S(O)₂R₈, tri-C₁₋₄alkylsilyl, C₃₋₆cycloalkyl, C₄₋₆heterocycloalkyl, C₃₋₆cycloalkenyl, C₄₋₆heterocycloalkenyl, phenyl, and C₅₋₆heteroaryl, provided when L₃ is a bond, R₀ is not hydrogen; wherein
   the C₁₋₆alkyl, C₂₋₄alkenyl, C₂₋₄alkynyl, or C₁₋₄alkoxy of R₀ is optionally substituted by 1 to 2-substituents independently selected from hydroxy, halo, C₁₋₄alkoxy, trimethylsilylC₁₋₄alkoxy, C₁₋₄alkoxycarbonyl, NR₁₁ₐR_{11b}, C₁₋₄alkoxycarbonylamino, C₅₋₆heterocycloalkyloxy, C₃₋₇cycloalkyl, C₅₋₆heterocycloalkyl, phenyl, and C₅₋₆heteroaryl, wherein the C₃₋₇cycloalkyl, C₅₋₆heterocycloalkyl, phenyl or C₅₋₆heteroaryl substituent is optionally substituted by hydroxy, halo, C₁₋₄alkyl, or C₁₋₄alkoxycarbonylamino, and wherein R₁₁ₐ and R_{11b} are independently hydrogen or C₁₋₄alkyl;
   the C₄₋₆heterocycloalkyloxy, C₃₋₆cycloalkyl, C₄₋₆heterocycloalkyl, C₄₋₆heterocycloalkenyl, phenyl, or C₅₋₆heteroaryl of R₀ is optionally substituted by 1 to 4 substituents independently selected from hydroxy, halo, oxo, C₁₋₄alkyl, C₁₋₄hydroxyalkyl, C₁₋₄haloalkyl, —(CH₂)₀₋₄NRₐR_{b}, C₅₋₆arylC₁₋₄alkyl, C₄₋₆heterocycloalkylC₁₋₄alkyl, C₁₋₄alkoxy, C₁₋₄alkoxycarbonyl, C₁₋₄alkylsulfonyl, C₄₋₆heterocycloalkyl, wherein Rₐ and R_{b} are each independently hydrogen, C₁₋₄alkyl or C₃₋₆cycloalkyl; wherein Rₐ and R_{b} are independently hydrogen, C₁₋₄alkyl or C₃₋₆cycloalkyl;
   R₂ₐ is selected from hydrogen, C₁₋₄alkyl, C₁₋₄alkoxyC₁₋₄alkyl, C₁₋₄alkylcarbonyloxyC₁₋₄alkyl, and a C₁₋₄haloalkyl substituted C₅₋₆heteroaryl;
   R_{2b} is selected from hydrogen, C₁₋₆alkyl, C₁₋₆haloalkyl, aminoC₁₋₄alkyl, C₁₋₄alkoxy, C₁₋₄alkoxycarbonyl, C₃₋₆cycloalkyl, C₄₋₆heterocycloalkyl, C₅₋₆heterocycloalkenyl, and C₅₋₆heteroaryl, wherein
      the C₁₋₄alkyl of R_{2b} is optionally substituted by C₁₋₄alkoxy, amino, C₁₋₄alkylamino, C₄₋₆cycloalkyl, C₄₋₆heterocycloalkyl, phenyl or C₅₋₆heteroaryl, wherein the C₄₋₆cycloalkyl, C₄₋₆heterocycloalkyl, phenyl or C₅₋₆heteroaryl substituent is optionally substituted by hydroxy, halo, C₁₋₄alkyl, C₁₋₄haloalkyl, or C₁₋₄alkyoxycarbonylamino, and
      the C₄₋₆heterocycloalkyl, C₅₋₆heterocycloalkenyl or C₅₋₆heteroaryl of R_{2b} is optionally substituted by 1 to 2 substituents independently selected from C₁₋₄alkyl and C₁₋₄haloalkyl;
   R₅ is hydrogen or C₁₋₄alkyl;
   R₆ is selected from hydrogen, C₁₋₆alkyl, C₁₋₆hydroxyalkyl, C₁₋₆haloalkyl, C₂₋₄alkenyl, C₁₋₄alkoxy, C₂₋₄alkenyloxy, C₃₋₆cycloalkyloxy, benzyloxy, —NR₁₂ₐR_{12b}, C₃₋₇cycloalkyl, C₄₋₇heterocycloalkyl, C₅₋₇cycloalkenyl, C₅₋₆heterocycloalkenyl, phenyl, and C₅₋₆heteroaryl, wherein
      R₁₂ₐ is hydrogen or C₁₋₆alkyl,
      R_{12b} is selected from hydrogen, C₁₋₆alkyl, amino and C₁₋₆alkylamino;
      the C₁₋₆alkyl, C₂₋₆alkenyl, C₁₋₄alkoxy or amino of R₆ is optionally substituted by 1 to 2 substituents independently selected from hydroxy, halo, C₁₋₄alkyl, C₁₋₄haloalkyl, C₁₋₄alkoxy, C₁₋₄haloalkoxy, C₁₋₄alkoxyC₁₋₄alkoxy, —NR₉ₐR_{9b}, C₃₋₆cycloalkyl, C₄₋₇heterocycloalkyl, and C₅₋₆heteroaryl, wherein the C₅₋₆heterocycloalkyl or C₅₋₆heteroaryl substituent is optionally substituted by 1-2 substituents independently selected from hydroxyl, C₁₋₄alkyl and C₁₋₄alkoxycarbonyl; and wherein R₉ₐ is hydrogen or C₁₋₄alkyl and R_{9b} is selected from hydrogen, C₁₋₄alkyl, C₁₋₄alkylcarbonyl and C₁₋₄alkoxycarbonyl; and
      the C₃₋₆cycloalkyloxy, C₃₋₆cycloalkyl, C₄₋₇heterocycloalkyl, C₅₋₇cycloalkenyl, or C₅₋₆heterocycloalkenyl of R₆ is optionally substituted by 1 to 3 substituents independently selected from hydroxy, halo, cyano, oxo, C₁₋₄alkyl, haloC₁₋₄alkyl, C₁₋₄alkoxylC₁₋₄alkyl, C₁₋₄alkoxycarbonylaminoC₁₋₄alkyl, C₁₋₄alkoxyl, C₁₋₄alkoxycarbonyl, amino, and aminocarbonyl,
      the phenyl or C₅₋₆heteroaryl of R₆ is optionally substituted by 1 to 2 substituents independently selected from hydroxy, halo, cyano, C₁₋₄alkyl, C₁₋₄hydroxyalkyl, C₁₋₄haloalkyl, C₁₋₄alkoxy, C₁₋₄alkoxycarbonyl, —(CH₂)₀₋₄NR₁₃ₐR_{13b}, and C₁₋₄alkoxycarbonylamino, wherein the C₁₋₄alkoxy substituent is optionally substituted by C₁₋₄alkylC₅₋₆heterocycloalkyl, and wherein R₁₃ₐ and R_{13b} are each independently hydrogen or C₁₋₄alkyl;
   R₈ is C₁₋₄alkyl or C₁₋₄alkylamino.

In yet another embodiment of the compound of Formula A of the invention,
Rₓ is hydrogen;
L₁ is a bond or —C(O)—;
R₁ is selected from C₁₋₆alkoxy, C₁₋₆alkoxyC₁₋₆alkoxy, NR₇ₐR_{7b}, C₃₋₆cycloalkyl, C₄₋₇heterocycloalkyl, C₅₋₆heterocycloalkenyl, phenyl, benzyl, and C₅₋₉heteroaryl, wherein
   R₇ₐ is hydrogen or C₁₋₄alkyl;
   R_{7b} is selected from hydrogen, C₁₋₄alkyl, C₁₋₄haloalkyl, C₁₋₄alkoxy, amino, C₁₋₄alkoxy substituted C₃₋₆cycloalkyl, C₃₋₆cycloalkyl, and C₅₋₆heteroaryl, wherein the C₅₋₆heteroaryl is optionally substituted by 1 to 2 substituents independently selected from halo and C₁₋₄alkyl;
   the C₃₋₆cycloalkyl, C₄₋₇heterocycloalkyl, C₅₋₆heterocycloalkenyl, phenyl, or C₅₋₉heteroary of R₁ is optionally substituted by 1-2 substituents independently selected from hydroxy, halo, cyano, oxo, C₁₋₄alkyl, C₁₋₄haloalkyl, C₁₋₄alkoxy, amino, C₁₋₄alkylamino, C₁₋₄alkylcarbonyl, am inocarbonyl, C₁₋₄alkylsulfonyl, C₃₋₆cycloalkyl, and C₄₋₆heterocycloalkyl;
R₃ is hydrogen or halo;
R₄ is hydrogen or halo;
L₃ is a selected from a bond, C₃₋₇cycloalkyl, C₄₋₇heterocycloalkyl, C₅₋₆cycloalkenyl, C₅₋₆heterocycloalkenyl, phenyl, and C₅₋₆heteroaryl;
R₀ is selected from hydrogen, halo, oxo, C₁₋₆alkyl, C₁₋₄haloalkyl, C₂₋₄alkenyl, C₂₋₄alkynyl, C₁₋₄alkoxy, C₄₋₆heterocycloalkyloxy, —C(O)R₆, —NR₂ₐR_{2b}, —NR₅C(O)R₆, -tri-C₁₋₄alkylsilyl, C₄₋₆heterocycloalkyl, C₄₋₆heterocycloalkenyl, phenyl, and C₅₋₆heteroaryl; provided when L₃ is a bond, R₀ is not hydrogen; wherein
   the C₁₋₄alkyl, C₂₋₄alkynyl or C₁₋₄alkoxy of R₀ is optionally substituted by 1 to 2 substituents independently selected from hydroxy, C₁₋₄alkoxy, trimethylsilylC₁₋₄alkoxy, C₁₋₄alkoxycarbonyl, -Nr₁₁ₐR_{11b}, C₁₋₄alkoxycarbonylamino, C₃₋₇cycloalkyl, C₅₋₆heterocycloalkyl, phenyl and C₅₋₆heteroaryl; wherein the C₃₋₇cycloalkyl or C₅₋₆heterocycloalkyl substituent is optionally substituted by hydroxy, C₁₋₄alkyl or C₁₋₄alkoxycarbonylamino, and wherein R₁₁ₐ and R_{11b} are independently hydrogen or C₁₋₄alkyl;
   the C₄₋₆heterocycloalkyloxy, C₄₋₆heterocycloalkyl, C₄₋₆heterocycloalkenyl, phenyl or C₅₋₆heteroaryl of R₀ is optionally substituted by 1 to 4 substituents independently selected from hydroxy, halo, C₁₋₄alkyl, C₁₋₄haloalkyl, —(CH₂)₀₋₄NRₐR_{b}, C₅₋₆arylC₁₋₄alkyl, C₄₋₆heterocycloalkylC₁₋₄alkyl, C₁₋₄alkoxycarbonyl, C₁₋₄alkylsulfonyl, and C₄₋₆heterocycloalkyl, wherein Rₐ and R_{b} are each independently hydrogen or C₁₋₄alkyl;
   R₂ₐ is selected from hydrogen, C₁₋₄alkyl, C₁₋₄alkoxyC₁₋₄alkyl, C₁₋₄alkylcarbonyloxyC₁₋₄alkyl, and a C₁₋₄haloalkyl substituted C₅₋₆heteroaryl;
   R_{2b} is selected from hydrogen, C₁₋₄alkyl, C₁₋₄haloalkyl, aminoC₁₋₄alkyl, C₁₋₄alkoxy, C₁₋₄alkoxycarbonyl, C₃₋₆cycloalkyl, C₄₋₆heterocycloalkyl, C₅₋₆heterocycloalkenyl, and C₅₋₆heteroaryl, wherein
      the C₁₋₄alkyl of R_{2b} is optionally substituted by C₁₋₄alkoxy, C₁₋₄alkylamino, C₄₋₆cycloalkyl, or C₅₋₆heteroaryl, wherein the C₄₋₆cycloalkyl substituent is optionally substituted by C₁₋₄alkyoxycarbonylamino; and
      the C₄₋₆heterocycloalkyl, C₅₋₆heterocycloalkenyl or C₅₋₆heteroaryl of R_{2b} is optionally substituted by 1 to 2 substituents independently selected from C₁₋₄alkyl and C₁₋₄haloalkyl;
   R₅ is hydrogen or C₁₋₄alkyl;
   R₆ is selected from hydrogen, C₁₋₆alkyl, C₁₋₆hydroxyalkyl, C₁₋₄haloalkyl,
   C₁₋₄alkoxy, C₂₋₄alkenyloxy, C₃₋₆cycloalkyloxy, benzyloxy, —NR₁₂ₐR_{12b}, C₃₋₇cycloalkyl, C₄₋₇heterocycloalkyl, C₅₋₇cycloalkenyl, C₅₋₆heterocycloalkenyl, phenyl, and C₅₋₆heteroaryl, wherein
      R₁₂ₐ is hydrogen or C₁₋₆alkyl;
      R_{12b} is selected from hydrogen, C₁₋₆alkyl, amino and C₁₋₆alkylamino;
      the C₁₋₆alkyl or C₁₋₄alkoxy of R₆ is optionally substituted by 1 to 2 substituents independently selected from halo, hydroxy, C₁₋₄haloalkyl, C₁₋₄alkoxy, C₁₋₄haloalkoxy, C₁₋₄alkoxyC₁₋₄alkoxy, —NR₉ₐR_{9b}, and C₄₋₇heterocycloalkyl, wherein R₉ₐ is hydrogen or C₁₋₄alkyl and R_{9b} is selected from hydrogen, C₁₋₄alkyl, and C₁₋₄alkoxycarbonyl;
      the C₃₋₆cycloalkyloxy, C₃₋₇cycloalkyl, C₄₋₇heterocycloalkyl, C₅₋₇cycloalkenyl, or C₅₋₆heterocycloalkenyl of R₆ is optionally substituted by 1 to 3 substituents independently selected from hydroxy, halo, cyano, oxo, C₁₋₆alkyl, C₁₋₄alkoxylC₁₋₄alkyl, C₁₋₄alkoxy, C₁₋₄alkoxycarbonyl, and amino;
      the phenyl or C₅₋₆heteroaryl of R₆ is optionally substituted with 1 to 2 substituents independently selected from halo, cyano, C₁₋₄alkyl, C₁₋₄hydroxyalkyl, C₁₋₄haloalkyl, C₁₋₄alkoxy, C₁₋₄alkoxycarbonyl, —(Ch₂)₀₋₄NR_{13A}R_{13B}, and C₁₋₄alkoxycarbonylamino, wherein the C₁₋₄alkoxy substituent is optionally substituted by C₁₋₄alkylC₅₋₆heterocycloalkyl, and wherein R₁₃ₐ and R_{13b} are each independently hydrogen or C₁₋₄alkyl; and
   R₈ is C₁₋₄alkyl.

In one variation of the above described embodiments of the compound of the invention, R₁ is selected from C₁₋₆alkoxy, C₃₋₆cycloalkyl, C₄₋₇heterocycloalkyl, C₅₋₆heterocycloalkenyl, phenyl, benzyl, and C₅₋₉heteroaryl, wherein the C₃₋₆cycloalkyl, C₄₋₇heterocycloalkyl, C₅₋₆heterocycloalkenyl, phenyl, or C₅₋₉heteroaryl is optional substituted by 1 to 2 substituents independently selected from hydroxy, halo, cyano, oxo, C₁₋₄alkyl, C₁₋₄haloalkyl, C₁₋₄alkoxy, amino, C₁₋₄alkylamino, C₁₋₄alkylcarbonyl, aminocarbonyl, C₁₋₄alkylsulfonyl, C₃₋₆cycloalkyl, and C₄₋₆heterocycloalkyl.

In another variation, R₁ is C₄₋₃heterocycloalkyl or C₃₋₆heteroaryl, each of which is optionally substituted by 1 to 2 substituents independently selected from halo and C₁₋₄alkyl.

In another variation, R₁ is selected from methoxy, ethoxy, isopropyloxy, cyclopropyl, cyclobutyl, cyclopentyl, cyclohexyl, azetidinyl, pyrrolidinyl, piperidinyl, piperazinyl, morpholinyl, tetrahydropyranyl, dioxanyl, 5-azaspiro[2,4]heptanyl, 2-oxa-6-aza-spiro[3,3]heptanyl, oxaazobicyclo[2.2.1]heptanyl, dihydrooxazolyl, phenyl, benzyl, pyrrolyl, pyrazolyl, imidazolyl, furanyl, isooxazolyl, oxazolyl, thiazolyl, pyridinyl, pyrimidinyl, indolyl, furo[2,3-c]pyridinyl, and imidazo[1,2-a]pyrimidinyl, wherein the cyclopropyl, cyclobutyl, cyclopentyl, cyclohexyl, azetidinyl, pyrrolidinyl, piperidinyl, piperazinyl, morpholinyl, tetrahydropyranyl, dioxanyl, 5-azaspiro[2,4]heptanyl, 2-oxa-6-aza-spiro[3,3]heptanyl, oxaazobicyclo[2.2.1]heptanyl, dihydrooxazolyl, phenyl, pyrrolyl, pyrazolyl, imidazolyl, furanyl, isooxazolyl, oxazolyl, thiazolyl, pyridinyl, pyrimidinyl, indolyl, furo[2,3-c]pyridinyl, or imidazo[1,2-a]pyrimidinyl, is optional substituted by 1 to 2 substituents independently selected from hydroxy, halo, cyano, oxo, methyl, trifluoromethyl, methoxy, amino, —NHCH₃, methylcarbonyl, aminocarbonyl, methylsulfonyl, cyclopropyl, and morpholinyl.

In another variation, R₁ is selected from azetidinyl, pyrrolidinyl, piperidinyl, piperazinyl, morpholinyl, tetrahydropyranyl, dioxanyl, pyrrolyl, pyrazolyl, imidazolyl, furanyl, isooxazolyl, oxazolyl, thiazolyl, pyridinyl, and pyrimidinyl, each of which is optional substituted by 1 to 2 substituents independently selected from hydroxy, halo, cyano, oxo, methyl, trifluoromethyl, methoxy, amino, and —NHCH₃.

In another variation, R₁ is selected from:

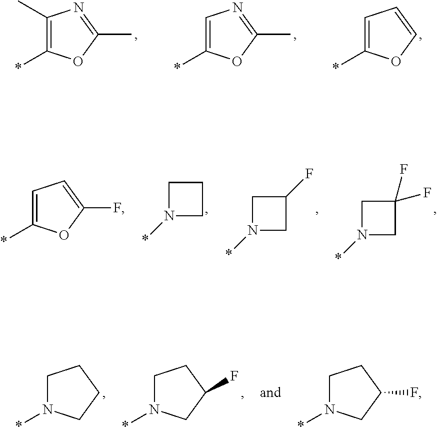

wherein “*” indicates the point of attachment of R₁.

In still another variation, R₁ is selected from:

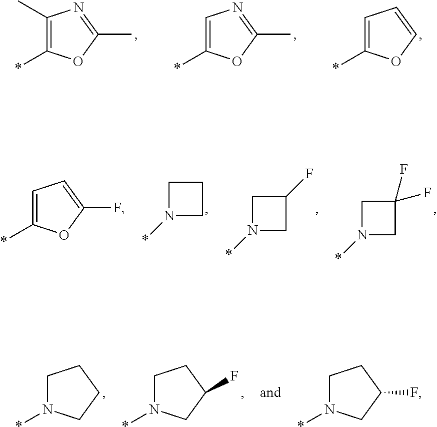

wherein “*” indicates the point of attachment of R₁. In still another variation, R1 is

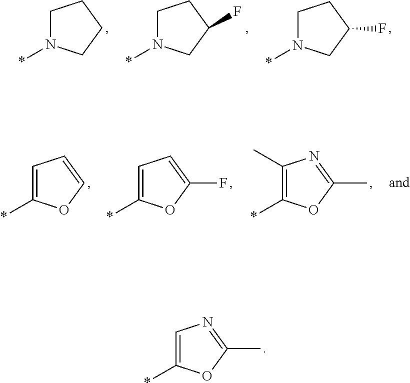

In still another variation, R₁ is

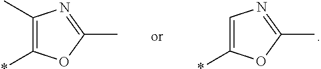

In still another variation, R₁ is

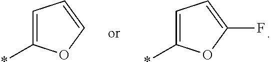 [SMILES: *c1ccco1 ; *c1ccc(F)o1]

In yet another variation, R₁ is selected from

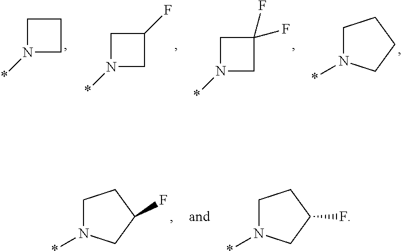

In yet another variation, R₁ is

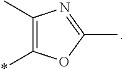 [SMILES: *c1oc(C)nc1C]

In yet another variation, R₁ is

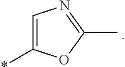 [SMILES: *c1cnc(C)o1]

In yet another variation, R₁ is

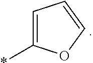 [SMILES: *c1ccco1]

In yet another variation, R₁ is

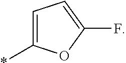 [SMILES: *c1ccc(F)o1]

In still yet another variation, R₁ is

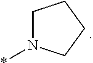 [SMILES: *N1CCCC1]

In still yet another variation, R₁ is

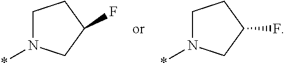 [SMILES: *N1CC[C@@H](F)C1 ; "or" ; *N1CC[C@H](F)C1]

In another variation of all of the above described embodiments and variations of the compound of the invention, L₃ is selected from a bond, C₃₋₇cycloalkyl, C₄₋₇heterocycloalkyl, C₄₋₇cycloalkenyl, C₅₋₇heterocycloalkenyl, phenyl, and C₅₋₆heteroaryl.

In another variation, L₃ is selected from C₃₋₇cycloalkyl, C₄₋₇heterocycloalkyl, C₄₋₇cycloalkenyl, and C₅₋₇heterocycloalkenyl. In another variation, L₃ is C₄₋₇heterocycloalkyl or C₅₋₇heterocycloalkenyl. In another variation, L₃ is phenyl or C₅₋₆heteroaryl.

In still another variation, L₃ is selected from cyclopropyl, cyclobutyl, cyclopentyl, bicyclo[3.1.1]heptanyl, pyrrolidinyl, piperidinyl, tetrahydropyranyl, morpholinyl, cyclopentenyl, tetrahydropyridinyl, dihydrofuranyl, dihydropyranyl, phenyl, pyrrolyl, pyrazolyl, imidazolyl, triazolyl, oxazolyl, isooxazolyl, pyridinyl, pyridazinyl, pyrimidinyl, and pyrazinyl. In still another variation, L₃ is selected from cyclopropyl, cyclobutyl, tetrahydropyranyl, cyclopentenyl, dihydrofuranyl, dihydropyranyl, phenyl, pyrazolyl, imidazolyl, thiazolyl, pyridinyl, and pyrimidinyl. In still another variation, L₃ is tetrahydropyranyl or dihydropyranyl. In still another variation, L₃ is phenyl, pyridinyl or pyrimidinyl.

In another variation of all of the above described embodiments and variations of the compound of the invention, R₀ is selected from hydrogen, halo, oxo, C₁₋₆alkyl, C₁₋₄hydroxyalkyl, C₁₋₄haloalkyl, C₂₋₄alkenyl, trimethylsilylC₁₋₆alkyloxyC₁₋₆alkyl, —NR₂ₐR_{2b}, —NHC(O) R₆, C₄₋₆heterocycloalkyl, C₄₋₆heterocycloalkenyl, phenyl and C₃₋₆heteroaryl, wherein

the C₄₋₆heterocycloalkyl, C₄₋₆heterocycloalkenyl, phenyl or C₃₋₆heteroaryl of R₀ is optionally substituted by 1 to 3 substituents independently selected from hydroxy, halo and C₁₋₄alkyl, C₁₋₄haloalkyl, and C₁₋₄alkoxycarbonyl;

R₂ₐ is selected from hydrogen, C₁₋₄alkyl, C₁₋₆alkoxyC₁₋₄alkyl, C₁₋₄alkylcarbonyloxyC₁₋₄alkyl, and a C₁₋₄haloalkyl substituted C₃₋₆heteroaryl;

R_{2b} is hydrogen, C₁₋₄alkyl, C₁₋₄alkoxy, or C₁₋₄alkoxycarbonyl;

R₆ is selected from C₁₋₄alkoxy, C₃₋₇cycloalkyl, C₄₋₇heterocycloalkyl, C₅₋₇cycloalkenyl, C₃₋₆heterocycloalkenyl, phenyl and C₃₋₆heteroaryl, wherein the C₃₋₇cycloalkyl, C₄₋₇heterocycloalkyl, C₃₋₇cycloalkenyl, or C₃₋₆heterocycloalkenyl is optionally substituted by 1 to 2 substituents independently selected from hydroxy, halo, cyano, oxo, C₁₋₆alkyl, C₁₋₄alkoxylC₁₋₄alkyl, C₁₋₄alkoxy, C₁₋₄alkoxycarbonyl, and amino, and wherein the phenyl or C₃₋₆heteroaryl of R₆ is optionally substituted by 1 to 2 substituents independently selected from halo, cyano, C₁₋₆alkyl, C₁₋₄hydroxyalkyl, C₁₋₄haloalkyl, C₁₋₄alkoxycarbonyl, amino, C₁₋₄alkyamino, and C₁₋₄alkoxycarbonylamino.

In another variation, R₀ is selected from hydrogen, halo, oxo, methyl, ethyl, isopropyl, isobutyl, —CH═CH₂, —(CH₂)₂OH, —CHF₂, —CF₃, —CH₂CF₃, —(CH₂)₂CF₃, trimethylsilylethoxymethyl, —NHCH₃, —NH-trifluoromethylpyridinyl, —N(CH₂OC(O)CH₂CH₃)(C(O)OCH(CH₃)₂), —NHC(O)R₆, azetidinyl, piperidinyl, piperazinyl, morpholinyl, dihydro-1,4-dioxinyl, dihydro-2H-pyranyl, phenyl, imidazolyl, oxazolyl, oxadiazoyl, thiazolyl, and pyridinyl, wherein the azetidinyl, piperidinyl, piperazinyl, morpholinyl, dihydro-1,4-dioxinyl, dihydro-2H-pyranyl, phenyl, imidazolyl, oxazolyl, oxadiazoyl, thiazolyl, or pyridinyl is optionally substituted by 1 to 3 substituents independently selected from hydroxy, halo, C₁₋₄alkyl, C₁₋₄haloalkyl, and —C(O)OCH₃, wherein

R₆ is selected from —OCH₃, —OCH(CH₃)₂, cyclopropyl, cyclobutyl, cyclohexyl, azetidinyl, pyrrolidinyl, piperidinyl, piperazinyl, oxetanyl, tetrahydrofuranyl, tetrahydropyranyl, morpholinyl, dioxanyl, oxazepanyl, oxabicyclo[2.2.1]heptanyl, bicyclo[2.2.1]hept-2-ene, dihydro-1,4-dioxinyl, phenyl, pyridinyl, imidazolyl, and triazolyl, wherein the cyclopropyl, cyclobutyl, cyclohexyl, azetidinyl, pyrrolidinyl, piperidinyl, piperazinyl, oxetanyl, tetrahydrofuranyl, tetrahydropyranyl, morpholinyl, dioxanyl, oxazepanyl, oxabicyclo[2.2.1]heptanyl, bicyclo[2.2.1]hept-2-ene, or dihydro-1,4-dioxinyl, of R₆ is optionally substituted by 1 to 3 substituents independently selected from hydroxy, halo, cyano, oxo, methyl, methoxymethyl, methoxy, butoxycarbonyl, amino, and wherein the phenyl, pyridinyl, imidazolyl, or triazolyl of R₆ is optionally substituted by halo, cyano, methyl, ethyl, isopropyl, —(CH₂)₂OH, —CHF₂, —CF₃, —CH₂CF₃, —(CH₂)₂CF₃, amino, —NHCH₃, —N(CH₃)₂, and butoxycarbonylamino.

In still another variation, R₀ is selected from hydrogen, halo, methyl, ethyl, isopropyl, isobutyl, —(CH₂)₂OH, —CHF₂, —CF₃, —CH₂CF₃, —(CH₂)₂CF₃, —NHCH₃, phenyl, and pyridinyl, wherein the phenyl or pyridinyl is optionally substituted by 1-2 substituents independently selected from halo and methyl. In still another variation, R₀ is selected from halo, methyl, ethyl, isopropyl, isobutyl, —(CH₂)₂OH, —CHF₂, —CF₃, —CH₂CF₃, —(CH₂)₂CF₃, —NHCH₃, phenyl, and pyridinyl, wherein the phenyl or pyridinyl is optionally substituted by 1-2 substituents independently selected from halo and methyl. In still another variation, R₀ is selected from methyl, ethyl, isopropyl, isobutyl, —(CH₂)₂OH, —CHF₂, —CF₃, —CH₂CF₃, —(CH₂)₂CF₃, and —NHCH₃. In still another variation, R₀ is halo. In still another variation, R₀ is phenyl or pyridinyl, optionally substituted by 1-2 substituents independently selected from halo and methyl.

In yet another variation of all of the above described embodiments and variations of the compound of the invention, -L₃-R₀ is selected from methyl, ethyl, isopropyl, —CHF₂, —CF₃, —CH₂CF₃, —(CH₂)₂CF₃,

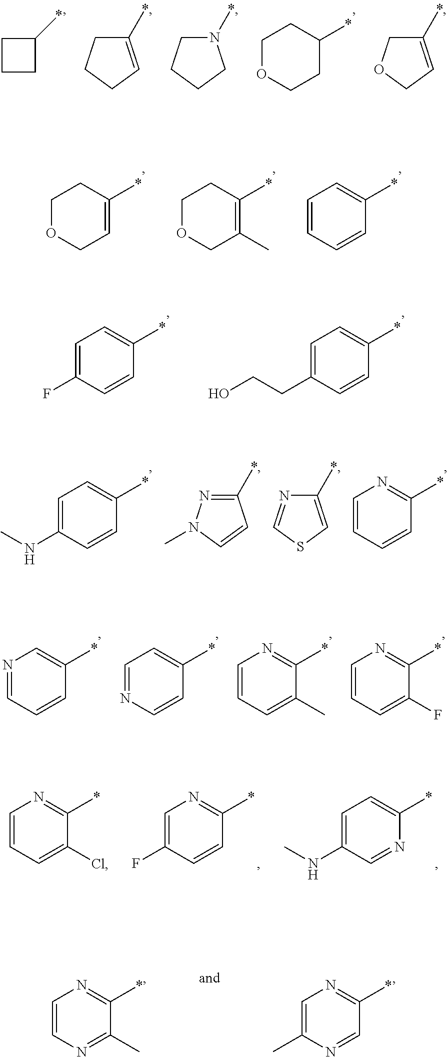

wherein “*” represents the point of attachment of -L₃R₀.

In another variation, -L₃-R₀ is C₁₋₄alkyl or C₁₋₄haloalkyl. In one variation, -L₃-R₀ is selected from methyl, ethyl, isopropyl, isobutyl, —CHF₂, —CF₃, —CH₂CF₃, and —(CH₂)₂CF₃. In another variation, -L₃-R₀ is C₁₋₄alkyl. In another variation, -L₃-R₀ is selected from —CHF₂, —CF₃, —CH₂CF₃, and —(CH₂)₂CF₃. In another variation, -L₃-R₀ is —CF₃ or CH₂CF₃. In another variation, -L₃-R₀ is CH₂CF₃. In another variation, -L₃-R₀ is —CF₃. In another variation, -L₃-R₀ is selected from

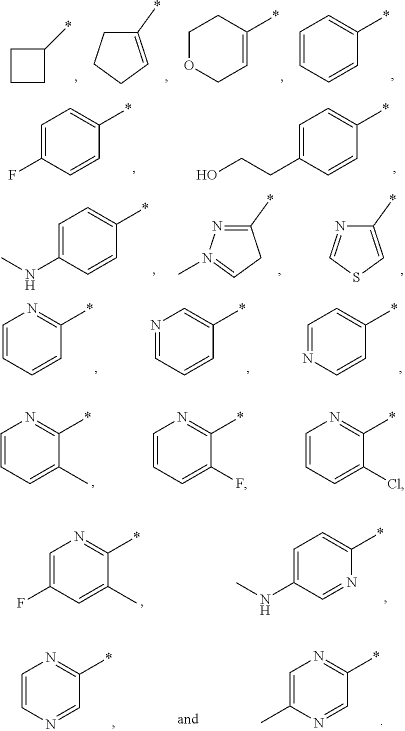

In still another variation, L₃-R₀ is selected from

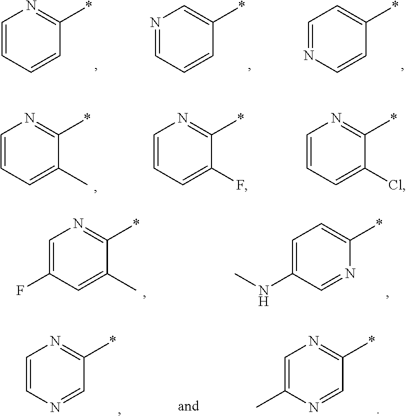

In still another variation, *-L₃-R₀ is selected from

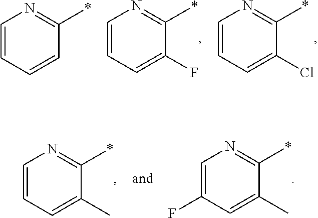

In still another variation, -L₃-R₀ is selected from

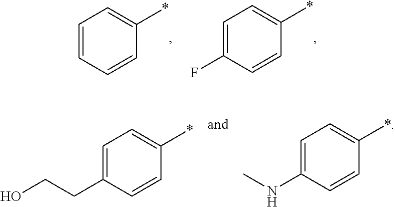

In still another variation, -L₃-R₀ is

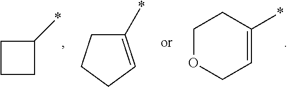

In yet another variation, -L₃-R₀ is

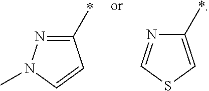

In a particular variation, -L₃-R₀ is selected from —CHF₂, —CF₃, —CH₂CF₃,

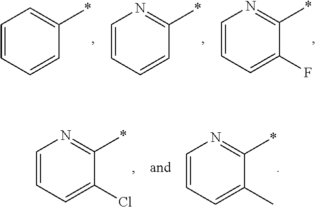

In another variation of all of the above described embodiments and variations, the compounds of the invention are those where R₃ is halo, and R₄ is hydrogen.

In a particular embodiment, the compound of the invention is of Formula A1:

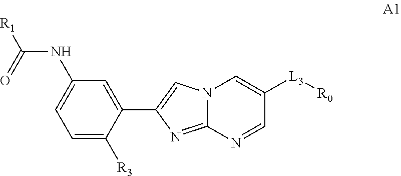

wherein

R₁ is C₄₋₇heterocycloalkyl or C₅₋₆heteroaryl, each of which is optionally substituted by 1-2 substituents independently selected from hydroxy, halo, cyano, oxo, C₁₋₄alkyl, C₁₋₄haloalkyl, C₁₋₆alkoxy, C₁₋₄alkylcarbonyl, aminocarbonyl, C₁₋₄alkylsulfonyl, amino, —NHCH₃, C₃₋₆cycloalkyl, and C₄₋₆heterocycloalkyl;

R₃ is hydrogen or halo;

L₃ is selected from a bond, C₃₋₇cycloalkyl, C₄₋₆heterocycloalkyl, C₅₋₆cycloalkenyl, C₅₋₆heterocycloalkenyl, phenyl, and C₅₋₆heteroaryl;

R₀ is selected from hydrogen, halo, C₁₋₆alkyl, C₁₋₄hydroxyalkyl, C₁₋₄haloalkyl, NR₂ₐR_{2b}, phenyl, and C₅₋₆heteroaryl; provided when L₃ is a bond, R₀ is not hydrogen; wherein the phenyl or C₅₋₆heteroaryl of R₀ is optionally substituted by 1 to 2 substituents independently selected from halo, C₁₋₄alkyl, C₁₋₄alkyamino, and C₁₋₄alkoxy, and

R₂ₐ and R_{2b} are each independently hydrogen or C₁₋₄alkyl.

In one variation of the above particular embodiment of the compound of the invention,

R₁ is C₄₋₇heterocycloalkyl or C₅₋₆heteroaryl, each of which is optionally substituted by 1 to 2 substituents independently selected from hydroxy, halo, cyano, oxo, C₁₋₄alkyl, C₁₋₄haloalkyl, C₁₋₆alkoxy, C₁₋₄alkylcarbonyl, aminocarbonyl, C₁₋₄alkylsulfonyl, amino, —NHCH₃, C₃₋₆cycloalkyl, and C₄₋₆heterocycloalkyl;

L₃ is selected from C₃₋₇cycloalkyl, C₄₋₆heterocycloalkyl, C₅₋₆cycloalkenyl, C₃₋₆heterocycloalkenyl, phenyl, and C₅₋₆heteroaryl;

R₀ is selected from hydrogen, halo, C₁₋₆alkyl, C₁₋₄hydroxyalkyl, C₁₋₄haloalkyl, NR₂ₐR_{2b}, phenyl, and C₅₋₆heteroaryl; wherein the phenyl or C₅₋₆heteroaryl is optionally substituted by 1 to 2 substituents independently selected from halo, C₁₋₄alkyl, C₁₋₄alkyamino, and C₁₋₄alkoxy, and

R₂ₐ and R_{2b} are each independently hydrogen or C₁₋₄alkyl.

In one variation of the above described particular embodiment and variations of the compound of the invention, R₁ is selected from azetidinyl, pyrrolidinyl, piperidinyl, piperazinyl, morpholinyl, tetrahydropyranyl, dioxanyl, pyrrolyl, pyrazolyl, imidazolyl, furanyl, isooxazolyl, oxazolyl, thiazolyl, pyridinyl, and pyrimidinyl, each of which is optional substituted by 1 to 2 substituents independently selected from hydroxy, halo, cyano, oxo, methyl, trifluoromethyl, methoxy, amino, and —NHCH₃.

In another variation, R₁ is selected from:

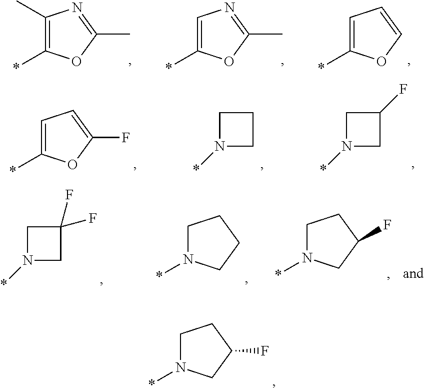

wherein “*” indicates the point of attachment of R₁.

In still another variation, R₁ is selected from:

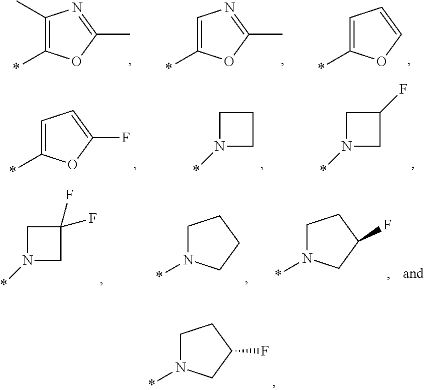

wherein “*” indicates the point of attachment of R₁. In still another variation, R₁ is

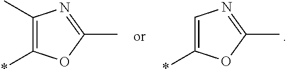

In still another variation, *—R₁ is

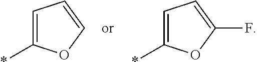 [SMILES: *c1ccco1 ; "or" ; *c1ccc(F)o1]

In yet another variation, R₁ is selected from

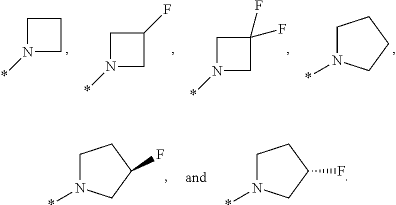

In yet another variation, R₁ is

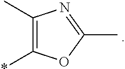 [SMILES: *c1oc(C)nc1C]

In yet another variation, R₁ is

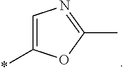 [SMILES: *c1cnc(C)o1]

In yet another variation, R₁ is

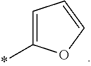 [SMILES: *c1ccco1]

In yet another variation, R₁ is

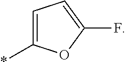 [SMILES: *c1ccc(F)o1]

In still yet another variation, R₁ is

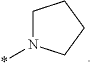 [SMILES: *N1CCCC1]

In still yet another variation, R₁ is

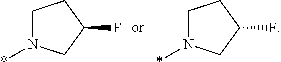 [SMILES: *N1CC[C@@H](F)C1 ; *N1CC[C@H](F)C1]

In one variation of the above described particular embodiment and variations of the compounds of the invention, L₃ is selected from C₃₋₇cycloalkyl, C₄₋₇heterocycloalkyl, C₄₋₇cycloalkenyl, and C₅₋₇heterocycloalkenyl. In another variation, L₃ is C₄₋₇heterocycloalkyl or C₅₋₇heterocycloalkenyl. In another variation, L₃ is phenyl or C₅₋₆heteroaryl.

In still another variation, L₃ is selected from cyclopropyl, cyclobutyl, cyclopentyl, bicyclo[3.1.1]heptanyl, pyrrolidinyl, piperidinyl, tetrahydropyranyl, morpholinyl, cyclopentenyl, tetrahydropyridinyl, dihydrofuranyl, dihydropyranyl, phenyl, pyrrolyl, pyrazolyl, imidazolyl, triazolyl, oxazolyl, isooxazolyl, pyridinyl, pyridazinyl, pyrimidinyl, and pyrazinyl. In still another variation, L₃ is selected from cyclopropyl, cyclobutyl, tetrahydropyranyl, cyclopentenyl, dihydrofuranyl, dihydropyranyl, phenyl, pyrazolyl, imidazolyl, thiazolyl, pyridinyl, and pyrimidinyl. In still another variation, L₃ is tetrahydropyranyl or dihydropyranyl. In still another variation, L₃ is phenyl, pyridinyl or pyrimidinyl.

In one variation of the above described particular embodiment and variations of the compound of the invention, R₀ is selected from hydrogen, halo, methyl, ethyl, isopropyl, isobutyl, —(CH₂)₂OH, —CHF₂, —CF₃, —CH₂CF₃, —(CH₂)₂CF₃, —NHCH₃, phenyl, and pyridinyl, wherein the phenyl or pyridinyl is optionally substituted by 1-2 substituents independently selected from halo and methyl. In yet another variation, R₀ is selected from halo, methyl, ethyl, isopropyl, isobutyl, —(CH₂)₂OH, —CHF₂, —CF₃, —CH₂CF₃, —(CH₂)₂CF₃, —NHCH₃, phenyl, and pyridinyl, wherein the phenyl or pyridinyl is optionally substituted by 1-2 substituents independently selected from halo and methyl. In yet another variation, R₀ is selected from methyl, ethyl, isopropyl, isobutyl, —(CH₂)₂OH, —CHF₂, —CF₃, —CH₂CF₃, —(CH₂)₂CF₃, and —NHCH₃. In yet another variation, R₀ is halo. In yet another variation, R₀ is phenyl or pyridinyl, optionally substituted by 1-2 substituents independently selected from halo and methyl.

In another variation of the above described particular embodiment and variations of the compounds of the invention, L₃-R₀ is selected from C₁₋₄alkyl or C₁₋₄haloalkyl,

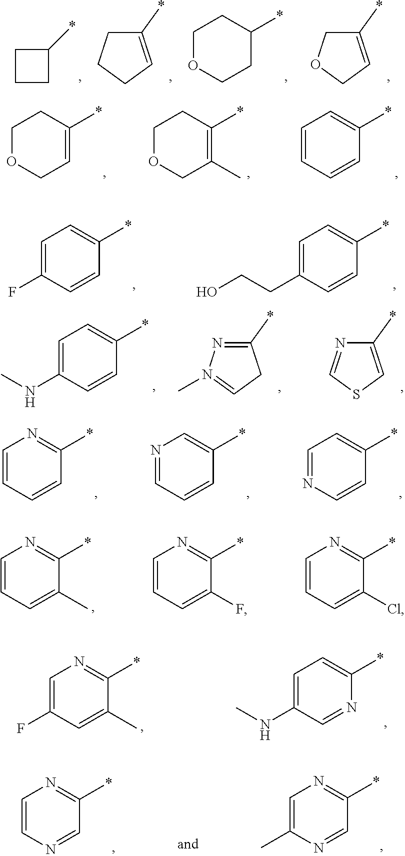

wherein “*” represents the point of attachment of -L₃R₀.

In another variation, -L₃-R₀ is C₁₋₄alkyl or C₁₋₄haloalkyl. In one variation, -L₃-R₀ is selected from methyl, ethyl, isopropyl, isobutyl, —CHF₂, —CF₃, —CH₂CF₃, and —(CH₂)₂CF₃. In another variation, -L₃-R₀ is C₁₋₄alkyl. In another variation, -L₃-R₀ is selected from —CHF₂, —CF₃, —CH₂CF₃, and —(CH₂)₂CF₃. In another variation, -L₃-R₀ is —CF₃ or CH₂CF₃. In [symbol] variation, -L₃-R₀ is selected from

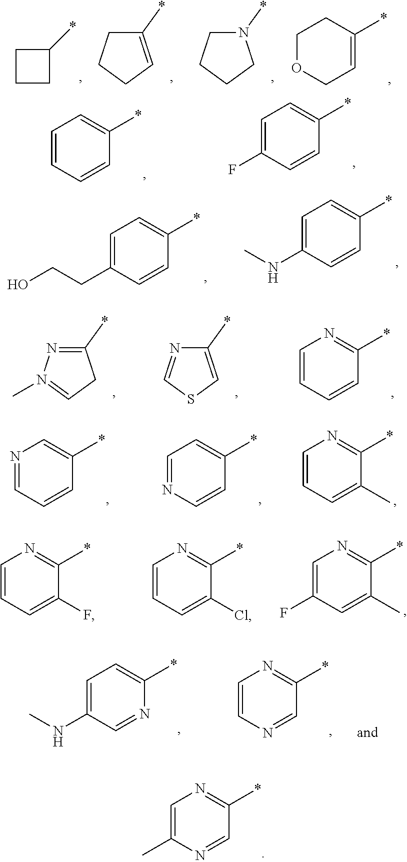

In still another variation, -L₃-R₀ is selected from

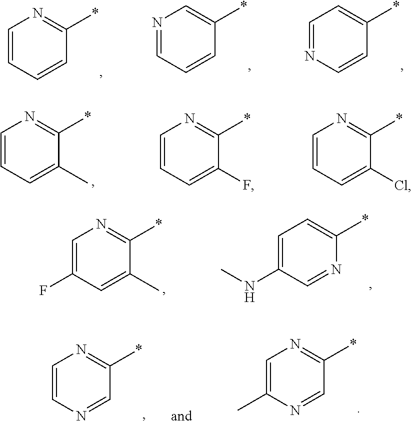

In still another variation, *-L₃-R₀ is selected from

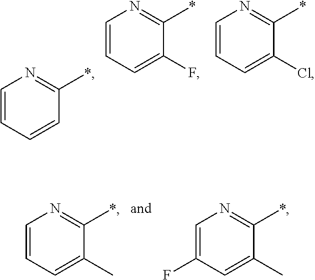

In still another variation, -L₃-R₀ is selected from

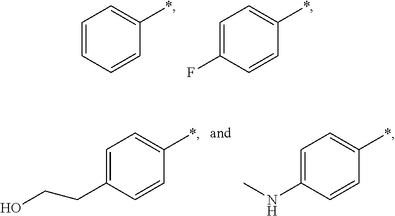

In still another variation, -L₃-R₀ is

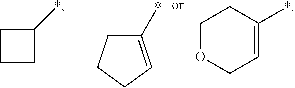

In yet another variation, -L₃-R₀ is

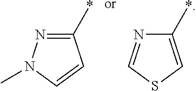

In a particular variation, -L₃-R₀ is selected from —CHF₂, —CF₃, —CH₂CF₃,

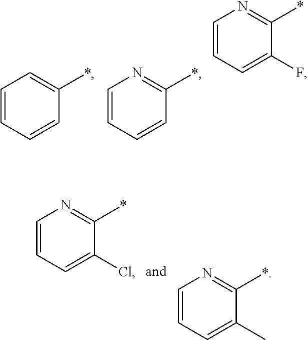

In another particular embodiment of the invention, the compound is of Formula I:

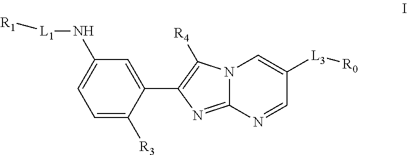

or a pharmaceutically acceptable salt, or stereoisomer thereof; wherein

L₁ is —C(O)— or —S(O)₂—;

R₁ is selected from nitro, C₁₋₄alkyl, C₁₋₆alkoxy, amino, C₅₋₉heteroaryl, C₃₋₆cycloalkyl and C₄₋₆heterocycloalkyl, each of which is optionally substituted by 1-2 substituent independently selected from halo, cyano, amino, C₁₋₄alkyl, haloC₁₋₄alkyl, C₁₋₆alkoxy, and C₁₋₄alkylcarbonyl; or —NHL₁R₁ is nitro;

R₃ is selected from hydrogen, halo, cyano, C₁₋₄alkyl and haloC₁₋₄alkyl;

R₄ is selected from hydrogen, C₁₋₄alkyl, haloC₁₋₄alkyl, and —C(O)R₁₀, wherein R₁₀ is hydroxy, C₁₋₄alkyl, C₁₋₄alkoxy, amino, C₁₋₄alkylamino, C₃₋₆cycloalkyl and C₄₋₆heterocycloalkyl, each of which is independently optionally substituted by 1-2 substituents independently selected from hydroxyl, halo and C₁₋₄alkyl;

L₃ is a bond, phenylene, or C₅₋₆heteroarylene;

R₀ is selected from hydrogen, hydroxyl, halo, nitro, —N═CHN(CH₃)₂, C₁₋₄alkyl, C₁₋₄alkoxy, —NR₂ₐR_{2b}, —NR₅C(O)R₆, —NR₅S(O)₂R₈, C₃₋₆cycloalkyl, C₄₋₆heterocycloalkyl, C₄₋₆heterocycloalkenyl, phenyl and C₅₋₆heteroaryl; wherein
the C₁₋₄alkyl or C₁₋₄alkoxy is optionally substituted by 1-2 substituents independently selected from C₁₋₄alkoxy, amino, phenyl and C₅₋₆heteroaryl; wherein the phenyl or C₅₋₆heteroaryl is optionally substituted by halo or C₁₋₄alkyl;
the C₃₋₆cycloalkyl, C₄₋₆heterocycloalkyl, C₄₋₆heterocycloalkenyl, phenyl and C₅₋₆heteroaryl of R₀ is optionally substituted with halo, oxo, C₁₋₄alkyl, hydroxyC₁₋₄alkYl, haloC₁₋₄alkyl, C₁₋₄alkoxy, amino, C₁₋₄alkylamino, and —(CH₂)₁₋₄NRₐR_{b}, wherein Rₐ and R_{b} are each independently hydrogen, C₁₋₄alkyl or C₃₋₆cycloalkyl;
R₂ₐ is hydrogen or C₁₋₄alkyl,
R_{2b} is selected from hydrogen, C₁₋₄alkyl, wherein the alkyl is optionally substituted by amino, C₄₋₆heterocycloalkyl, phenyl or C₅₋₆heteroaryl, wherein the C₄₋₆heterocycloalkyl, phenyl or C₅₋₆heteroaryl is optionally substituted by hydroxyl, halo or C₁₋₄alkyl;
R₅ is hydrogen or C₁₋₄alkyl;
R₆ is selected from hydrogen, C₁₋₆alkyl, C₁₋₄alkoxy, C₃₋₆cycloalkoxy, amino, C₃₋₆cycloalkyl, C₅₋₆heterocycloalkyl, and C₅₋₆heteroaryl, wherein
   the C₁₋₄alkyl, C₁₋₄alkoxy, C₃₋₆cycloalkoxy, and amino of R₆ are each optionally substituted by 1-2 substituents independently selected from halo, hydroxy, C₁₋₄alkyl, haloC₁₋₄alkyl, C₁₋₄alkoxy, —NR₉ₐR_{9b}, C₃₋₆cycloalkyl, C₅₋₆heterocycloalkyl, and C₅₋₆heteroaryl, wherein R₉ₐ is hydrogen or C₁₋₄alkyl and R_{9b} is selected from hydrogen, C₁₋₄alkyl, C₁₋₄alkylcarbonyl and C₁₋₄alkoxycarbonyl, and the C₅₋₆heterocycloalkyl and C₅₋₆heteroaryl are each optionally substituted by 1-2 substituents independently selected from hydroxyl, C₁₋₄alkyl and C₁₋₄alkoxycarbonyl,
   the C₅₋₆heteroaryl of R₆ is optionally substituted with 1-2 substituents selected from hydroxy, C₁₋₄alkyl, and C₁₋₄alkoxycarbonyl,
   the C₃₋₆cycloalkyl or C₄₋₆heterocycloalkyl of R₆ are each independently optionally substituted by 1-2 substituents independently selected from halo, cyano, hydroxy, C₁₋₄alkyl, haloC₁₋₄alkyl, C₁₋₄alkoxylC₁₋₄alkyl, aminocarbonyl, C₁₋₄alkoxycarbonyl, and C₁₋₄alkoxycarbonylaminoC₁₋₄alkyl, and
R₈ is C₁₋₄alkyl or C₁₋₄alkylamino.

In another embodiment of the above embodiment of the compound of the invention, L₁ is —C(O)—.

In another embodiment of the above embodiments, in one variation, R₁ is selected from C₁₋₄alkyl, C₁₋₆alkoxy, amino, C₅₋₉heteroaryl, C₃₋₆cycloalkyl and C₄₋₆heterocycloalkyl, each is optionally substituted by 1-2 substituent independently selected from hydroxyl, halo, cyano, amino, C₁₋₄alkyl, haloC₁₋₄alkyl, C₁₋₆alkoxy, C₁₋₄alkylcarbonyl, phenyl and C₅₋₆heteroaryl.

In another variation, R₁ is selected from C₁₋₆alkoxy and C₁₋₆alkylamino, wherein the C₁₋₆alkoxy and C₁₋₆alkylamino are each optionally substituted by 1-2 substituents independently selected from C₁₋₄alkyl and C₁₋₄alkoxy.

In another variation, R₁ is selected from —CH₃, —(CH₂)₁₋₃CH₃, —CH(CH₃)₂, —CH₂CH(CH₃)₂, —(CH₂)₂F, —(CH₂)₂OCH₃, —N(CH₃)₂, —N(CH₃)CH₂CH₃, —N(CH₂CH₃)₂, —N(CH₃)OCH₃, —OCH₂CH₃, —O(CH₂)₃CH₃, —OCH(CH₃)₂, —OCH₂CH(CH₃)₂, —O(CH₂)₂OCH₃.

In still another variation, R₁ is selected from —N(CH₃)CH₂CH₃, —N(CH₃)₂, —N(CH₂CH₃)₂, —N(CH₃)OCH₃, —OCH₂CH₃, —OCH(CH₃)₂, —O(CH₂)₂OCH₃.

In still another variation, R₁ is selected from C₅₋₉heteroaryl, C₄₋₆heterocycloalkyl, and C₃₋₆cycloalkyl, each of which is independently optionally substituted by 1-2 substituents independently selected from halo, cyano, C₁₋₄alkyl and C₁₋₄alkoxy.

In yet another variation, R₁ is selected from pyrrolyl, pyrazolyl, imidazolyl, oxazolyl, furanyl, thiophenyl, thiazolyl, phenyl, pyrazinyl, cyclopropyl, cyclopentyl, pyrrolidinyl, and indolyl, each of which is independently optionally substituted by 1-2 substituents independently selected from halo, cyano, C₁₋₄alkyl, haloC₁₋₄alkyl and C₁₋₄alkylcarbonyl.

In another embodiment of first embodiment above, in one variation, L₁-R₁ is selected from —C(O)CH(CH₃)₂, —C(O)(CH₂)₂F, —C(O)CH(NH₂)(CH₃), —C(O)N(CH₃)₂, —C(O)N(CH₃)CH₂CH₃, —C(O)N(CH₂CH₃)₂, —C(O)N(CH₃)OCH₃, —C(O)OCH₂CH₃, —C(O)OCH(CH₃)₂, —C(O)OCH(CH₃)(CH₂CH₃), —C(O)O(CH₂)CH(CH₃)₂, —C(O)O(CH₂)₂OCH₃. —S(O)₂CH₃, and —S(O)₂CH(CH₃)₂.

In another variation, L₁-R₁ is selected from —NHC(O)N(CH₃)CH₂CH₃, —NHC(O)N(CH₃)OCH₃, —NHC(O)N(CH₃)₂, —NHC(O)N(CH₂CH₃)₂, —NHC(O)OCH₂CH₃, —NHC(O)OCH(CH₃)₂, and —NHC(O)O(CH₂)₂OCH₃.

In another variation, L₁-R₁ is selected from

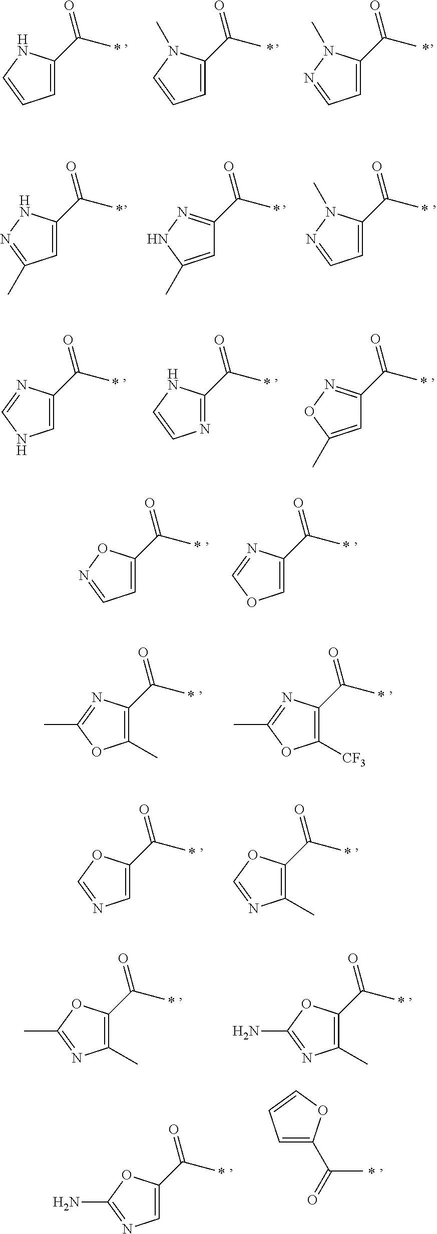

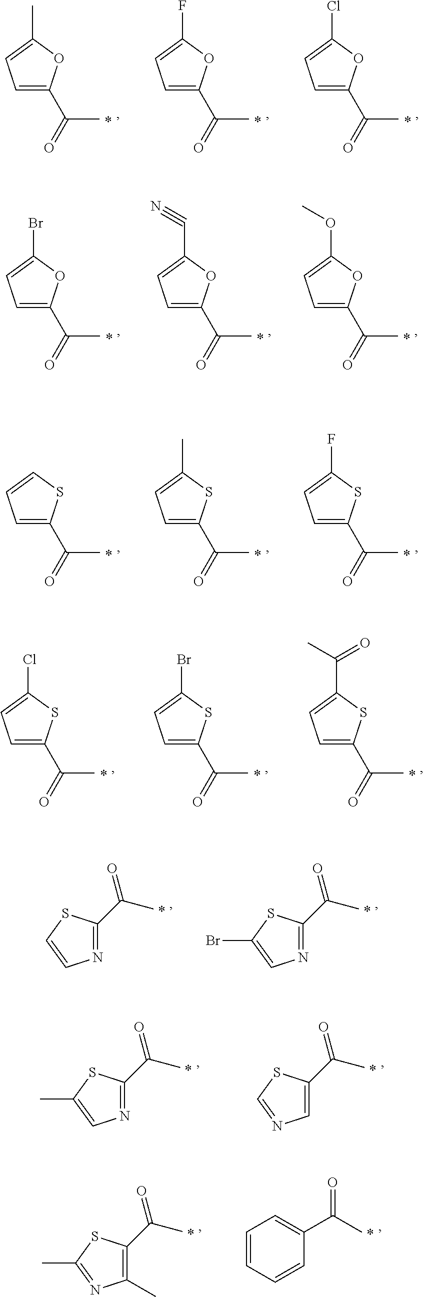

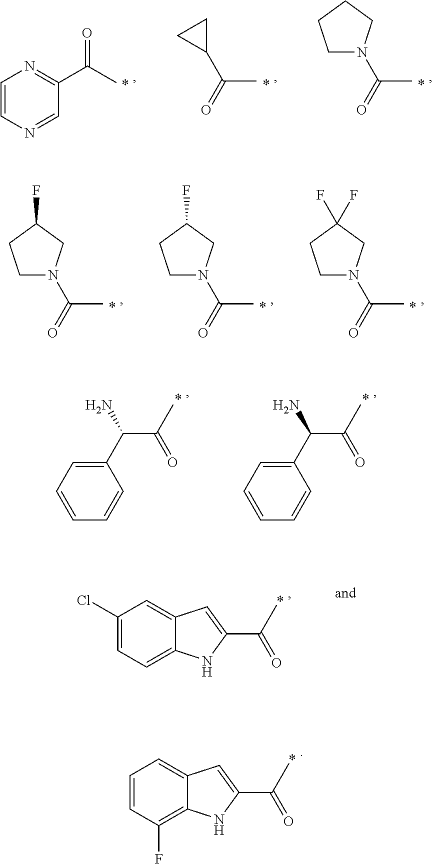

In still another variation, L₁-R₁ is selected from —C(O)OCH₂CH₃, —C(O)O(CH₂)₂OCH₃,

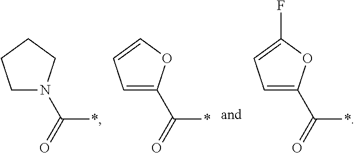

In another embodiment of the first embodiment of the compounds of the invention, NHL₁R₁ is nitro.

In still another embodiment of the above embodiments and variations of the compound of the invention, R₃ is selected from hydrogen, halo, methyl, or trifluoromethyl. In one variation, R₃ is chloro or fluoro. In another variation, R₃ is methyl or trifluorormethyl. In yet another variation, R₃ is hydrogen.

In still another embodiment of the above embodiments and variations of the compound of the invention, R₄ is hydrogen, fluoro, ethyl, —C(O)OCH(CH₃)(CH₂CH₃), and —C(O)OCH(CH₃)₂. In one variation, R₄ is halo. In another variation, R₄ is C₁₋₄alkyl. In still another variation, R₄ is hydrogen.

In yet another embodiment of the above embodiments and variations of the compound of the invention, in one variation, R₀ is selected from hydrogen, halo, nitro, —N═CHN(CH₃)₂, NHR_{2b}, —NR₅C(O)R₆, —NR₅S(O)₂R₈, C₄₋₆heterocycloalkyl, C₄₋₆heterocycloalkenyl, phenyl and C₅₋₆heteroaryl; wherein

the C₄₋₆heterocycloalkyl, C₄₋₆heterocycloalkenyl, phenyl and C₅₋₆heteroaryl of R₀ is optionally substituted with oxo, C₁₋₄alkyl, —(CH₂)₁₋₄OH, and —(CH₂)₁₋₄NRₐR_{b}, wherein Rₐ and R_{b} are each independently hydrogen, C₁₋₄alkyl or C₃₋₈cycloalkyl;

R_{2b} is selected from hydrogen, C₁₋₄alkyl, wherein the alkyl is optionally substituted by amino, C₄₋₆heterocycloalkyl, phenyl or C₅₋₆heteroaryl, wherein the C₄₋₆heterocycloalkyl, phenyl or C₅₋₆heteroaryl is optionally substituted by hydroxy or halo;

R₅ is hydrogen or C₁₋₄alkyl;

R₆ is selected from hydrogen, C₁₋₈alkyl, C₁₋₄alkoxy, C₃₋₈cycloalkoxy, amino, C₃₋₆cycloalkyl, C₅₋₆heterocycloalkyl, and C₅₋₆heteroaryl, wherein
the C₁₋₄alkyl, C₁₋₄alkoxy, C₃₋₈cycloalkoxy, and amino of R₆ are each optionally substituted by 1-2 substituents independently selected from halo, hydroxy, C₁₋₄alkyl, haloC₁₋₄alkyl, C₁₋₄alkoxy, —NR₉ₐR_{9b}, C₅₋₆heterocycloalkyl, and C₅₋₆heteroaryl, wherein R₉ₐ is hydrogen or C₁₋₄alkyl, and R_{9b} is selected from hydrogen, C₁₋₄alkyl, and C₁₋₄alkoxycarbonyl; and the C₅₋₆heterocycloalkyl and C₅₋₆heteroaryl are each optionally substituted by 1-2 substituents independently selected from hydroxyl, C₁₋₄alkyl and C₁₋₄alkoxycarbonyl;
the C₅₋₆heteroaryl of R₆ is optionally substituted with 1 to 2 C₁₋₄alkyl;
the C₃₋₈cycloalkyl or C₄₋₆heterocycloalkyl of R₆ are each independently optionally substituted by 1-2 substituents independently selected from halo, cyano, hydroxy, C₁₋₄alkyl, haloC₁₋₄alkyl, —CH₂OCH₃, —C(O)NH₂, C₁₋₄alkoxycarbonyl and C₁₋₄alkoxycarbonylaminoC₁₋₄alkyl; and

R₈ is C₁₋₄alkyl or C₁₋₄alkylamino.

In another variation of the above embodiment, R₀ is selected from hydrogen, halo, nitro, hydroxyl, C₁₋₄alkoxy, amino, C₁₋₄alkylamino, —NH(CH₂)₁₋₂-phenyl, —NR₅C(O)R₆, —NR₅S(O)₂R₈, oxazolidin-2-one, 1,2,4-triazol-5(4H)-one, pyrrolidin-2-one, phenyl and C₅₋₆heteroaryl; wherein

the oxazolidin-2-one, 1,2,4-triazol-5(4H)-one, pyrrolidin-2-one, phenyl or C₅₋₆heteroaryl is optionally substituted with halo, C₁₋₄alkyl, C₁₋₄alkoxy, amino, C₁₋₄alkylamino, —(CH₂)₁₋₄OH, and —(CH₂)₁₋₄NRₐR_{b}, wherein Rₐ and R_{b} are each independently hydrogen, C₁₋₄alkyl or C₃₋₈cycloalkyl;

R₅ is hydrogen or C₁₋₄alkyl;

R₆ is selected from C₁₋₈alkyl, C₁₋₈alkoxyl, C₃₋₈cycloalkyl, C₅₋₆heterocycloalkyl, and C₅₋₆heteroaryl, each of which is optionally substituted with 1 to 2 substituents independently selected from hydroxyl, C₁₋₄alkoxy, amino, C₁₋₄alkylamino; and

R₈ is C₁₋₄alkyl or C₁₋₄alkylamino.

In another variation of the above embodiment, R₀ is selected from hydrogen, fluoro, chloro, nitro, methyl, —NH₂, —NH(CH₃), —NH(CH₂CH₃), —N(CH₃)₂, —NHCH₂C(CH₃)₂NH₂, —NH(CH₂)₁₋₂-4-fluorophenyl, —NH-pyridin-3-yl, —NHCH₂-pyridin-4-yl, —NHCH₂-2-hydroxypyridin-3-yl, —NHCH₂-piperidin-4-yl, phenyl, thiophenyl, imidazolyl, oxazolidin-2-one, 1,2,4-triazol-5(4H)-one, and pyrrolidin-2-one, wherein the oxazolidin-2-one, 1,2,4-triazol-5(4H)-one, and pyrrolidin-2-one are each optionally substituted by C₁₋₄alkyl, —(CH₂)₁₋₄OH, and —(CH₂)₁₋₄NRₐR_{b}, wherein Rₐ and R_{b} are each independently hydrogen, C₁₋₄alkyl or C₃₋₈cycloalkyl.

In yet another variation of the above embodiment, R₀ is —NR₅C(O)R₆, wherein

R₅ is hydrogen or C₁₋₄alkyl;

R₆ is hydrogen, C₁₋₄alkyl, C₁₋₄alkoxy, amino, C₃₋₈cycloalkyloxy, C₃₋₈cycloalkyl, C₅₋₆heterocycloalkyl, and C₅₋₆heteroaryl, wherein
the C₁₋₄alkyl, C₁₋₄alkoxy, amino and C₃₋₈cycloalkyloxy are each optionally substituted by 1-2 substituents independently selected from halo, hydroxyl, C₁₋₄alkyl, haloC₁₋₄alkyl, C₁₋₄alkoxy, —NH₂, C₁₋₄alkylamino, —NHC(O)OC(CH₃)₃, pyrrolidinyl, piperidinyl, morpholinyl, and pyridinyl, wherein the pyrrolidinyl, piperidinyl, morpholinyl, or pyridinyl are each optionally substituted by hydroxy, C₁₋₄alkyl, or —C(O)OC(CH₃)₃);
the C₅₋₆heteroaryl is optionally substituted with 1-2 substituents independently selected from hydroxyl and C₁₋₄alkyl;
the C₃₋₆cycloalkyl or C₄₋₆heterocycloalkyl, each of which is independently optionally substituted by 1-2 substituents independently selected from halo, cyano, hydroxy, methyl, trifluoromethyl, —CH₂OCH₃, —CH₂NHC(O)(O)C(CH₃)₃, —C(O)(O)C(CH₃)₃, and —C(O)NH₂.

In yet still another variation of the above embodiment, R₀ is —NHC(O)R₆, wherein R₆ is selected from hydrogen, methyl, ethyl propyl, isopropyl, butyl, isobutyl, tert-butyl, —(CH₂)NH₂, —(CH₂)₂NH₂, —(CH₂)₃NH₂, —CH₂C(CH₃)₂NH₂, —CH(CH₃)NH₂, —C(CH₃)₂NH₂, —CH₂N(CH₃)₂, —(CH₂)₂NHC(O)OC(CH₃)₃, —(CH₂)-piperidin-4-yl, —CH₂-2-hydroxypiperidin-3-yl, —(CH₂)-pyrrolidin-3-yl, —CH₂-(1-tert-butoxycarbonyl)pyrrolidin-3-yl, —(CH₂)₂₋₃-morpholinyl, —(CH₂)-pyridin-3-yl, —(CH₂)₂OH, —C(CH₃)₂CH₂OH, —CH(OH)CH₂OH, —(CH₂)₂OCH₃; —OCH₃, —OCH₂CH₃, —OCH₂CH₂CH₃, —OCH(CH₃)₂, —OCH(CH₃)(CH₂CH₃), 1-methylcycopropoxy, —O(CH₂)₂F, —OC(CH₃)₂NH₂, —OCH₂C(CH₃)₂NH₂, —O(CH₂)₂OCH₃, —O(CH₂)₂—NHC(O)OC(CH₃)₃, —OCH₂C(CH₃)₂—NHC(O)OC(CH₃)₃, —NH₂, —NH(CH₃), —NHCH(CH₃)₂, —N(CH₃)₂, and —N(CH₃)CH(CH₃)₂, and —NH-pyridin-3-yl.

In yet still another variation of the above embodiment, R₀ is —NHC(O)R₆, wherein R₆ is selected from thiazolyl, pyridinyl, cyclopropyl, cyclobutyl, azetidinyl, pyrrolidinyl, pyrrolidinyl, piperidinyl, and oxetanyl, each of which is independently optionally substituted with 1-2 substituents independently selected from fluoro, cyano, hydroxy, C₁₋₄alkyl, trifluoromethyl, —CH₂NHC(O)OC(CH₃)₃, —C(O)NH₂, —CH₂O(CH₃), and —C(O)OC(CH₃)₃

In yet still another variation of the above embodiment, R₀ is —NHS(O)₂R₈, wherein R₈ is C₁₋₄alkyl or C₁₋₄alkylamino.

In yet still another variation of the above embodiment, R₀ is —NHS(O)₂R₈, wherein R₈ is methyl, isopropyl, methylamino or dimethylamino.

In yet another embodiment of the above embodiments and variations of the compounds of the invention, in one variation, -L₃R₀ is selected from chloro, bromo, nitro, —NHC(O)OCH(CH₃)₂, —N(CH₂CH₃)C(O)OCH(CH₃)₂, NHC(O)OCH₃, —NHC(O)N(CH₃)₂, phenyl, and thiophen-3-yl.

In another variation of the above embodiment, L₃R₃ is selected from —NH—C(O)CH(CH₃)₂, —NH—C(O)-cyclopropyl, —NH—C(O)O-cyclopropyl, —NH—C(O)-cyclobutyl, wherein the cyclopropyl and cyclobutyl are each independently optionally substituted by a substituent independently selected from cyano, halo and C₁₋₄alkyl.

In another variation, -L₃R₀ is selected from —NHC(O)OCH(CH₃)₂, —NHC(O)OCH₂CH₃, —NHC(O)OCH₃, nitro,

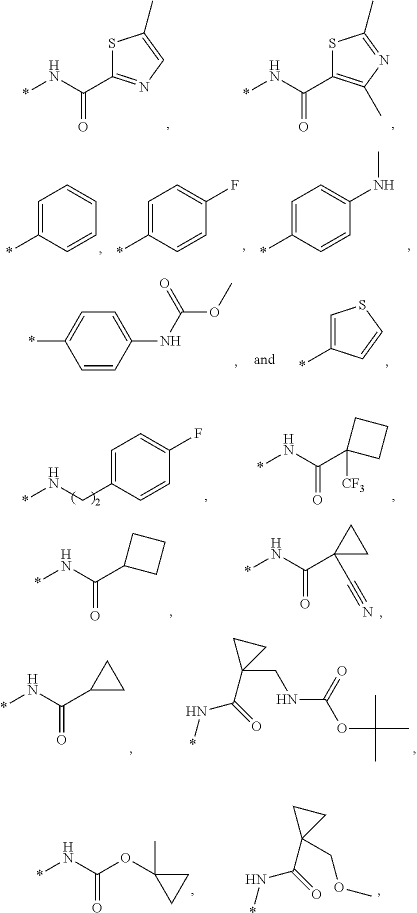

In yet another embodiment, -L₃R₀ is —NHC(O)OCH(CH₃)₂.

Compounds of the invention include, but are not limited to, the exemplified compounds listed in Table II (pages 232 to 354), and the corresponding chemical names of the compounds are listed below. It is understood that when there is a discrepancy between the structure and the name of a compound, the structure governs.

Accordingly, the compounds of the invention includes: propan-2-yl N-{2-[2-chloro-5-(5-fluorofuran-2-amido)phenyl]imidazo[1,2-a]pyrimidin-6-yl}carbamate; propan-2-yl N—(2-{2-chloro-5-[(pyrrolidin-1-yl)carbonylamino]phenyl}imidazo[1,2-a]pyrimidin-6-yl)carbamate; propan-2-yl N-{2-[3-(5-fluorofuran-2-amido)phenyl]imidazo[1,2-a]pyrimidin-6-yl}carbamate; propan-2-yl N—(2-{2-chloro-5-[(3,3-difluoropyrrolidin-1-yl)carbonylamino]phenyl}imidazo[1,2-a]pyrimidin-6-yl)carbamate; propan-2-yl N-(2-{3-[(pyrrolidin-1-yl)carbonylamino]phenyl}imidazo[1,2-a]pyrimidin-6-yl)carbamate; propan-2-yl N-[2-(3-{[ethyl(methyl)carbamoyl]amino}phenyl) imidazo[1,2-a]pyrimidin-6-yl]carbamate; propan-2-yl N-{2-[3-(pyrazine-2-amido)phenyl]imidazo[1,2-a]pyrimidin-6-yl}carbamate; propan-2-yl N-{2-[3-(furan-2-amido)phenyl]imidazo[1,2-a]pyrimidin-6-yl}carbamate; propan-2-yl N-[2-(5-{[ethyl(methyl)carbamoyl]amino}-2-fluorophenyl)imidazo[1,2-a]pyrimidin-6-yl]carbamate; propan-2-yl N-(2-{3-[(ethoxycarbonyl)amino]phenyl}imidazo[1,2-a]pyrimidin-6-yl)carbamate; propan-2-yl N-{2-[3-(1,3-thiazole-2-amido)phenyl]imidazo[1,2-a]pyrimidin-6-yl}carbamate; propan-2-yl N-{2-[2-fluoro-5-(furan-2-amido)phenyl]imidazo[1,2-a]pyrimidin-6-yl}carbamate; propan-2-yl N-{2-[3-(5-methylfuran-2-amido)phenyl]imidazo[1,2-a]pyrimidin-6-yl}carbamate; propan-2-yl N-[2-(3-{[methoxy(methyl)carbamoyl]amino}phenyl)imidazo[1,2-a]pyrimidin-6-yl]carbamate; propan-2-yl N-(2-{2-chloro-5-[(pyrrolidin-1-yl)carbonylamino]phenyl}-3-fluoroimidazo[1,2-a]pyrimidin-6-yl)carbamate; propan-2-yl N-(2-{3-[(diethylcarbamoyl)amino]phenyl}imidazo[1,2-a]pyrimidin-6-yl)carbamate; propan-2-yl N-(2-{2-fluoro-5-[(pyrrolidin-1-yl)carbonylamino]phenyl}imidazo[1,2-a]pyrimidin-6-yl)carbamate; propan-2-yl N-{2-[3-(5-chlorofuran-2-amido)phenyl]imidazo[1,2-a]pyrimidin-6-yl}carbamate; propan-2-yl N-[2-(2-fluoro-5-nitrophenyl)imidazo[1,2-a]pyrimidin-6-yl]carbamate; propan-2-yl N-(2-{3-[(dimethylcarbamoyl)amino]phenyl}imidazo[1,2-a]pyrimidin-6-yl)carbamate; propan-2-yl N-[2-(3-{[(propan-2-yloxy)carbonyl]amino}phenyl)imidazo[1,2-a]pyrimidin-6-yl]carbamate; propan-2-yl N-{2-[3-(2,4-dimethyl-1,3-oxazole-5-amido)phenyl]imidazo[1,2-a]pyrimidin-6-yl}carbamate; propan-2-yl N-[2-(3-{[(2-methoxyethoxy)carbonyl]amino}phenyl)imidazo[1,2-a]pyrimidin-6-yl]carbamate; methyl N-[4-(2-{2-chloro-5-[(pyrrolidin-1-yl)carbonylamino]phenyl}imidazo[1,2-a]pyrimidin-6-yl)phenyl]carbamate; N-{4-chloro-3-[6-(4-fluorophenyl)imidazo[1,2-a]pyrimidin-2-yl]phenyl}pyrrolidine-1-carboxamide; N-(4-chloro-3-{6-[4-(methylamino)phenyl]imidazo[1,2-a]pyrimidin-2-yl}phenyl)pyrrolidine-1-carboxamide; methyl N-(4-{2-[2-chloro-5-(5-methylfuran-2-amido)phenyl]imidazo[1,2-a]pyrimidin-6-yl}phenyl)carbamate; N-{4-chloro-3-[6-(thiophen-3-yl)imidazo[1,2-a]pyrimidin-2-yl]phenyl}pyrrolidine-1-carboxamide; N-(4-chloro-3-{6-phenylimidazo[1,2-a]pyrimidin-2-yl}phenyl)pyrrolidine-1-carboxamide; N-{4-chloro-3-[6-(thiophen-3-yl)imidazo[1,2-a]pyrimidin-2-yl]phenyl}-5-methylfuran-2-carboxamide; N-(4-chloro-3-{6-phenylimidazo[1,2-a]pyrimidin-2-yl}phenyl)-5-methylfuran-2-carboxamide; 2-methoxyethyl N-[4-chloro-3-(6-{4-[(methoxycarbonyl)amino]phenyl}imidazo[1,2-a]pyrimidin-2-yl)phenyl]carbamate; N-(4-chloro-3-{6-[4-(methylamino)phenyl]imidazo[1,2-a]pyrimidin-2-yl}phenyl)-5-methylfuran-2-carboxamide; 2-methoxyethyl N-{4-chloro-3-[6-(thiophen-3-yl)imidazo[1,2-a]pyrimidin-2-yl]phenyl}carbamate; 2-methoxyethyl N-(4-chloro-3-{6-phenylimidazo[1,2-a]pyrimidin-2-yl}phenyl)carbamate; 2-methoxyethyl N-(4-chloro-3-{6-[4-(methylamino)phenyl]imidazo[1,2-a]pyrimidin-2-yl}phenyl)carbamate; ethyl N-(4-chloro-3-{6-[4-(methylamino)phenyl]imidazo[1,2-a]pyrimidin-2-yl}phenyl)carbamate; N-(4-chloro-3-{6-chloroimidazo[1,2-a]pyrimidin-2-yl}phenyl)furan-2-carboxamide; N-(4-chloro-3-{3-fluoro-6-phenylimidazo[1,2-a]pyrimidin-2-yl}phenyl)pyrrolidine-1-carboxamide; propan-2-yl N-[2-(2-fluoro-5-{[(propan-2-yloxy)carbonyl]amino}phenyl)imidazo[1,2-a]pyrimidin-6-yl]carbamate; propan-2-yl N-{2-[3-(5-methyl-1,3-thiazole-2-amido)phenyl]imidazo[1,2-a]pyrimidin-6-yl}carbamate; propan-2-yl N-[2-(3-cyclopropaneamidophenyl)imidazo[1,2-a]pyrimidin-6-yl]carbamate; propan-2-yl N-{2-[2-chloro-5-(1,3-oxazole-4-amido)phenyl]imidazo[1,2-a]pyrimidin-6-yl}carbamate; propan-2-yl N-{2-[2-chloro-5-(5-chlorofuran-2-amido)phenyl]imidazo[1,2-a]pyrimidin-6-yl}carbamate; propan-2-yl N-{2-[2-chloro-5-(dimethyl-1,3-oxazole-5-amido)phenyl]imidazo[1,2-a]pyrimidin-6-yl}carbamate; propan-2-yl N-{2-[2-chloro-5-(dimethyl-1,3-oxazole-4-amido)phenyl]imidazo[1,2-a]pyrimidin-6-yl}carbamate; propan-2-yl N-{2-[2-chloro-5-(2-methyl-1,3-oxazole-5-amido)phenyl]imidazo[1,2-a]pyrimidin-6-yl}carbamate; propan-2-yl N-{2-[2-chloro-5-(5-cyanofuran-2-amido)phenyl]imidazo[1,2-a]pyrimidin-6-yl}carbamate; propan-2-yl N-[2-(2-chloro-5-{imidazo[1,2-a]pyrimidine-2-amido}phenyl)imidazo[1,2-a]pyrimidin-6-yl]carbamate; propan-2-yl N-{2-[2-chloro-5-(2-acetamido-1,3-thiazole-5-amido)phenyl]imidazo[1,2-a]pyrimidin-6-yl}carbamate; propan-2-yl N-{2-[2-chloro-5-(2-chloro-1,3-thiazole-5-amido)phenyl]imidazo[1,2-a]pyrimidin-6-yl}carbamate; propan-2-yl N-{2-[2-chloro-5-(1,3-oxazole-5-amido)phenyl]imidazo[1,2-a]pyrimidin-6-yl}carbamate; propan-2-yl N-{2-[2-chloro-5-(4-methyl-1,3-oxazole-5-amido)phenyl]imidazo[1,2-a]pyrimidin-6-yl}carbamate; propan-2-yl N-{2-[2-chloro-5-(2-methoxypropanamido)phenyl]imidazo[1,2-a]pyrimidin-6-yl}carbamate; propan-2-yl N-{2-[2-chloro-5-(oxolane-2-amido)phenyl]imidazo[1,2-a]pyrimidin-6-yl}carbamate; 1-{[4-chloro-3-(6-{[(propan-2-yloxy)carbonyl]amino}imidazo[1,2-a]pyrimidin-2-yl)phenyl]carbamoyl}ethyl acetate; propan-2-yl N-{2-[2-chloro-5-(oxane-2-amido)phenyl]imidazo[1,2-a]pyrimidin-6-yl}carbamate; propan-2-yl N-(2-{2-chloro-5-[2-(2,2,2-trifluoroethoxy)propanamido]phenyl}imidazo[1,2-a]pyrimidin-6-yl)carbamate; propan-2-yl N-{2-[2-chloro-5-(2-methyl-1H-imidazole-4-amido)phenyl]imidazo[1,2-a]pyrimidin-6-yl}carbamate; propan-2-yl N-{2-[2-chloro-5-(2-cyclopropyl-1,3-oxazole-5-amido)phenyl]imidazo[1,2-a]pyrimidin-6-yl}carbamate; propan-2-yl N-{2-[5-(dimethyl-1,3-oxazole-5-amido)-2-fluorophenyl]imidazo[1,2-a]pyrimidin-6-yl}carbamate; propan-2-yl N-{2-[5-(dimethyl-1,3-oxazole-4-amido)-2-fluorophenyl]imidazo[1,2-a]pyrimidin-6-yl}carbamate; propan-2-yl N-{2-[2-fluoro-5-(5-fluorofuran-2-amido)phenyl]imidazo[1,2-a]pyrimidin-6-yl}carbamate; propan-2-yl N-{2-[5-(5-chlorofuran-2-amido)-2-fluorophenyl]imidazo[1,2-a]pyrimidin-6-yl}carbamate; propan-2-yl N-{2-[2-fluoro-5-(1,3-oxazole-4-amido)phenyl]imidazo[1,2-a]pyrimidin-6-yl}carbamate; propan-2-yl N-{2-[5-(5-chlorothiophene-2-amido)-2-fluorophenyl]imidazo[1,2-a]pyrimidin-6-yl}carbamate; propan-2-yl N-{2-[2-fluoro-5-(5-fluorothiophene-2-amido)phenyl]imidazo[1,2-a]pyrimidin-6-yl}carbamate; propan-2-yl N-{2-[5-(dimethyl-1,3-thiazole-5-amido)-2-fluorophenyl]imidazo[1,2-a]pyrimidin-6-yl}carbamate; propan-2-yl N-(2-{2-fluoro-5-[2-methyl-5-(trifluoromethyl)-1,3-oxazole-4-amido]phenyl}imidazo[1,2-a]pyrimidin-6-yl)carbamate; propan-2-yl N-{2-[5-(5-bromofuran-2-amido)-2-fluorophenyl]imidazo[1,2-a]pyrimidin-6-yl}carbamate; propan-2-yl N-{2-[2-fluoro-5-(1H-pyrrole-2-amido)phenyl]imidazo[1,2-a]pyrimidin-6-yl}carbamate; propan-2-yl N-{2-[2-fluoro-5-(1,2-oxazole-5-amido)phenyl]imidazo[1,2-a]pyrimidin-6-yl}carbamate; propan-2-yl N-{2-[2-fluoro-5-(1-methyl-1H-pyrrole-2-amido)phenyl]imidazo[1,2-a]pyrimidin-6-yl}carbamate; propan-2-yl N-{2-[2-fluoro-5-(1-methyl-1H-pyrazole-5-amido)phenyl]imidazo[1,2-a]pyrimidin-6-yl}carbamate; propan-2-yl N-{2-[2-fluoro-5-(thiophene-2-amido)phenyl]imidazo[1,2-a]pyrimidin-6-yl}carbamate; propan-2-yl N-{2-[2-fluoro-5-(4-methyl-1,3-oxazole-5-amido)phenyl]imidazo[1,2-a]pyrimidin-6-yl}carbamate; propan-2-yl N-{2-[2-fluoro-5-(5-methyl-1H-pyrazole-3-amido)phenyl]imidazo[1,2-a]pyrimidin-6-yl}carbamate; propan-2-yl N-{2-[2-fluoro-5-(1,3-oxazole-5-amido)phenyl]imidazo[1,2-a]pyrimidin-6-yl}carbamate; propan-2-yl N-{2-[2-fluoro-5-(3-methyl-1H-pyrazole-5-amido)phenyl]imidazo[1,2-a]pyrimidin-6-yl}carbamate; propan-2-yl N-{2-[2-fluoro-5-(5-methylfuran-2-amido)phenyl]imidazo[1,2-a]pyrimidin-6-yl}carbamate; propan-2-yl N-{2-[2-fluoro-5-(1H-imidazole-4-amido)phenyl]imidazo[1,2-a]pyrimidin-6-yl}carbamate; propan-2-yl N-{2-[5-(5-bromothiophene-2-amido)-2-fluorophenyl]imidazo[1,2-a]pyrimidin-6-yl}carbamate; propan-2-yl N-{2-[5-(5-acetylthiophene-2-amido)-2-fluorophenyl]imidazo[1,2-a]pyrimidin-6-yl}carbamate; propan-2-yl N-(2-{5-[(2S)-2-amino-2-phenylacetamido]-2-fluorophenyl}imidazo[1,2-a]pyrimidin-6-yl)carbamate; propan-2-yl N-(2-{5-[(2R)-2-amino-2-phenylacetamido]-2-fluorophenyl}imidazo[1,2-a]pyrimidin-6-yl)carbamate; propan-2-yl N-{2-[2-fluoro-5-(5-methyl-1,2-oxazole-3-amido)phenyl]imidazo[1,2-a]pyrimidin-6-yl}carbamate; propan-2-yl N-{2-[5-(2-aminopropanamido)-2-fluorophenyl]imidazo[1,2-a]pyrimidin-6-yl}carbamate; propan-2-yl N-{2-[2-fluoro-5-(5-methylthiophene-2-amido)phenyl]imidazo[1,2-a]pyrimidin-6-yl}carbamate; propan-2-yl N-{2-[5-(5-chloro-1H-indole-2-amido)-2-fluorophenyl]imidazo[1,2-a]pyrimidin-6-yl}carbamate; propan-2-yl N-{2-[2-fluoro-5-(1,3-thiazole-5-amido)phenyl]imidazo[1,2-a]pyrimidin-6-yl}carbamate; propan-2-yl N-{2-[5-(2-bromo-1,3-thiazole-5-amido)-2-fluorophenyl]imidazo[1,2-a]pyrimidin-6-yl}carbamate; propan-2-yl N-{2-[2-fluoro-5-(1H-imidazole-2-amido)phenyl]imidazo[1,2-a]pyrimidin-6-yl}carbamate; propan-2-yl N-{2-[2-fluoro-5-(7-fluoro-1H-indole-2-amido)phenyl]imidazo[1,2-a]pyrimidin-6-yl}carbamate; propan-2-yl N-{2-[5-(2-amino-1,3-oxazole-5-amido)-2-fluorophenyl]imidazo[1,2-a]pyrimidin-6-yl}carbamate; propan-2-yl N-{2-[2-fluoro-5-(2-methyl-1,3-oxazole-5-amido)phenyl]imidazo[1,2-a]pyrimidin-6-yl}carbamate; propan-2-yl N-{2-[2-fluoro-5-(1-methyl-1H-imidazole-5-amido)phenyl]imidazo[1,2-a]pyrimidin-6-yl}carbamate; propan-2-yl N-{2-[2-fluoro-5-(1-methyl-1H-imidazole-2-amido)phenyl]imidazo[1,2-a]pyrimidin-6-yl}carbamate; propan-2-yl N-{2-[2-fluoro-5-(1-methyl-1H-pyrazole-3-amido)phenyl]imidazo[1,2-a]pyrimidin-6-yl}carbamate; propan-2-yl N-{2-[2-fluoro-5-(1,3-oxazole-2-amido)phenyl]imidazo[1,2-a]pyrimidin-6-yl}carbamate; propan-2-yl N-{2-[5-(2-chloro-1H-imidazole-5-amido)-2-fluorophenyl]imidazo[1,2-a]pyrimidin-6-yl}carbamate; propan-2-yl N-{2-[2-fluoro-5-(3-oxocyclobutaneamido)phenyl]imidazo[1,2-a]pyrimidin-6-yl}carbamate; propan-2-yl N-[2-(5-cyclobutaneamido-2-fluorophenyl)imidazo[1,2-a]pyrimidin-6-yl]carbamate; propan-2-yl N-(2-{5-[(2E)-4-(dimethylamino)but-2-enamido]-2-fluorophenyl}imidazo[1,2-a]pyrimidin-6-yl)carbamate; propan-2-yl N-{2-[2-fluoro-5-(2-methylbutanamido)phenyl]imidazo[1,2-a]pyrimidin-6-yl}carbamate; propan-2-yl N-{2-[2-fluoro-5-(2-methylpropanamido)phenyl]imidazo[1,2-a]pyrimidin-6-yl}carbamate; propan-2-yl N-{2-[2-fluoro-5-(2-methoxyacetamido)phenyl]imidazo[1,2-a]pyrimidin-6-yl}carbamate; propan-2-yl N-{2-[5-(5-cyanofuran-2-amido)-2-fluorophenyl]imidazo[1,2-a]pyrimidin-6-yl}carbamate; propan-2-yl N-{2-[5-(2-cyclopropyl-1,3-oxazole-5-amido)-2-fluorophenyl]imidazo[1,2-a]pyrimidin-6-yl}carbamate; propan-2-yl N-{2-[2-fluoro-5-(5-methanesulfonylthiophene-2-amido)phenyl]imidazo[1,2-a]pyrimidin-6-yl}carbamate; propan-2-yl N-(2-{2-fluoro-5-[2-(pyrrolidin-1-yl)acetamido]phenyl}imidazo[1,2-a]pyrimidin-6-yl)carbamate; propan-2-yl N-{2-[2-fluoro-5-(4-methyl-1,2-oxazole-5-amido)phenyl]imidazo[1,2-a]pyrimidin-6-yl}carbamate; propan-2-yl N-{2-[2-fluoro-5-(3-methyl-1,2-oxazole-5-amido)phenyl]imidazo[1,2-a]pyrimidin-6-yl}carbamate; N-{3-[6-(1,4-dioxane-2-amido)imidazo[1,2-a]pyrimidin-2-yl]-4-fluorophenyl}-2,4-dimethyl-1,3-oxazole-5-carboxamide; N-{4-fluoro-3-[6-(1-methylcyclobutaneamido)imidazo[1,2-a]pyrimidin-2-yl]phenyl}-2,4-dimethyl-1,3-oxazole-5-carboxamide; N-{4-fluoro-3-[6-(1-methylcyclopropaneamido)imidazo[1,2-a]pyrimidin-2-yl]phenyl}-2,4-dimethyl-1,3-oxazole-5-carboxamide; N-{4-fluoro-3-[6-(3-methyl-5,6-dihydro-1,4-dioxine-2-amido)imidazo[1,2-a]pyrimidin-2-yl]phenyl}-2,4-dimethyl-1,3-oxazole-5-carboxamide; N-{3-[6-(1-ethyl-1H-imidazole-2-amido)imidazo[1,2-a]pyrimidin-2-yl]-4-fluorophenyl}-2,4-dimethyl-1,3-oxazole-5-carboxamide; N-{3-[6-(3,3-difluoro-1-methylcyclobutaneamido)imidazo[1,2-a]pyrimidin-2-yl]-4-fluorophenyl}-2,4-dimethyl-1,3-oxazole-5-carboxamide; N-{2-[5-(dimethyl-1,3-oxazole-5-amido)-2-fluorophenyl]imidazo[1,2-a]pyrimidin-6-yl}pyridine-2-carboxamide; N-(4-fluoro-3-{6-[2-(propan-2-yl)oxane-4-amido]imidazo[1,2-a]pyrimidin-2-yl}phenyl)-2,4-dimethyl-1,3-oxazole-5-carboxamide; N-{4-fluoro-3-[6-(pyrrolidine-2-amido)imidazo[1,2-a]pyrimidin-2-yl]phenyl}pyrrolidine-1-carboxamide; N-{4-fluoro-3-[6-(3-methyl-5,6-dihydro-1,4-dioxine-2-amido)imidazo[1,2-a]pyrimidin-2-yl]phenyl}pyrrolidine-1-carboxamide; N-(2-{2-fluoro-5-[(pyrrolidine-1-carbonyl)amino]phenyl}imidazo[1,2-a]pyrimidin-6-yl)-2,4-dimethyl-1,3-oxazole-5-carboxamide; 5-fluoro-N-(2-{2-fluoro-5-[(pyrrolidine-1-carbonyl)amino]phenyl}imidazo[1,2-a]pyrimidin-6-yl)pyridine-2-carboxamide; N-(2-{2-fluoro-5-[(pyrrolidine-1-carbonyl)amino]phenyl}imidazo[1,2-a]pyrimidin-6-yl)-5-oxopyrrolidine-2-carboxamide; N-(4-fluoro-3-{6-[(2R)-oxolane-2-amido]imidazo[1,2-a]pyrimidin-2-yl}phenyl)pyrrolidine-1-carboxamide; N-{4-fluoro-3-[6-(2-methyloxolane-2-amido)imidazo[1,2-a]pyrimidin-2-yl]phenyl}pyrrolidine-1-carboxamide; N-(4-fluoro-3-{6-[2-hydroxy-2-(trifluoromethyl)butanamido]imidazo[1,2-a]pyrimidin-2-yl}phenyl)pyrrolidine-1-carboxamide; N-{4-fluoro-3-[6-(oxane-2-amido)imidazo[1,2-a]pyrimidin-2-yl]phenyl}pyrrolidine-1-carboxamide; (2S)—N-(2-{2-fluoro-5-[(pyrrolidine-1-carbonyl)amino]phenyl}imidazo[1,2-a]pyrimidin-6-yl)morpholine-2-carboxamide; N-(4-fluoro-3-{6-[1-(methoxymethyl)cyclopropaneamido]imidazo[1,2-a]pyrimidin-2-yl}phenyl)pyrrolidine-1-carboxamide; N-(2-{2-fluoro-5-[(pyrrolidine-1-carbonyl)amino]phenyl}imidazo[1,2-a]pyrimidin-6-yl)-2-methyl-1,3-oxazole-5-carboxamide; N-{4-fluoro-3-[6-(1-methylcyclopropaneamido)imidazo[1,2-a]pyrimidin-2-yl]phenyl}pyrrolidine-1-carboxamide; N-(3-{6-cyclopropaneamidoimidazo[1,2-a]pyrimidin-2-yl}-4-fluorophenyl)pyrrolidine-1-carboxamide; N-(4-fluoro-3-{6-[(2S)-oxolane-2-amido]imidazo[1,2-a]pyrimidin-2-yl}phenyl)pyrrolidine-1-carboxamide; N-{4-fluoro-3-[6-(2-methyloxane-2-amido)imidazo[1,2-a]pyrimidin-2-yl]phenyl}pyrrolidine-1-carboxamide; N-(4-fluoro-3-{6-[2-(2-methoxyethoxy)acetamido]imidazo[1,2-a]pyrimidin-2-yl}phenyl)pyrrolidine-1-carboxamide; N-{3-[6-(2-ethoxyacetamido)imidazo[1,2-a]pyrimidin-2-yl]-4-fluorophenyl}pyrrolidine-1-carboxamide; N-{4-fluoro-3-[6-(2-methoxyacetamido)imidazo[1,2-a]pyrimidin-2-yl]phenyl}pyrrolidine-1-carboxamide; N-{4-fluoro-3-[6-(1-hydroxycyclopropaneamido)imidazo[1,2-a]pyrimidin-2-yl]phenyl}pyrrolidine-1-carboxamide; N-{4-fluoro-3-[6-(3-oxocyclobutaneamido)imidazo[1,2-a]pyrimidin-2-yl]phenyl}pyrrolidine-1-carboxamide; 1-ethyl-N-(2-{2-fluoro-5-[(pyrrolidine-1-carbonyl)amino]phenyl}imidazo[1,2-a]pyrimidin-6-yl)-1H-imidazole-2-carboxamide; N-{3-[6-(1,4-dioxane-2-amido)imidazo[1,2-a]pyrimidin-2-yl]-4-fluorophenyl}pyrrolidine-1-carboxamide; N-(2-{2-fluoro-5-[(pyrrolidine-1-carbonyl)amino]phenyl}imidazo[1,2-a]pyrimidin-6-yl)-1-methyl-1H-imidazole-2-carboxamide; N-{4-fluoro-3-[6-(1-methylcyclobutaneamido)imidazo[1,2-a]pyrimidin-2-yl]phenyl}pyrrolidine-1-carboxamide; N-{3-[6-(3,3-difluoro-1-methylcyclobutaneamido)imidazo[1,2-a]pyrimidin-2-yl]-4-fluorophenyl}pyrrolidine-1-carboxamide; N-(2-{2-fluoro-5-[(pyrrolidine-1-carbonyl)amino]phenyl}imidazo[1,2-a]pyrimidin-6-yl)pyridine-2-carboxamide; N-{4-fluoro-3-[6-(4-methyl-1,3-thiazole-2-amido)imidazo[1,2-a]pyrimidin-2-yl]phenyl}-2,4-dimethyl-1,3-oxazole-5-carboxamide; N-{4-fluoro-3-[6-(oxolane-2-amido)imidazo[1,2-a]pyrimidin-2-yl]phenyl}-2,4-dimethyl-1,3-oxazole-5-carboxamide; N-{2-[5-(dimethyl-1,3-oxazole-5-amido)-2-fluorophenyl]imidazo[1,2-a]pyrimidin-6-yl}-2,4-dimethyl-1,3-oxazole-5-carboxamide; N-{4-fluoro-3-[6-(oxane-2-amido)imidazo[1,2-a]pyrimidin-2-yl]phenyl}-2,4-dimethyl-1,3-oxazole-5-carboxamide; N-{2-[5-(dimethyl-1,3-oxazole-5-amido)-2-fluorophenyl]imidazo[1,2-a]pyrimidin-6-yl}-4-methylmorpholine-2-carboxamide; N-{3-[6-(1-cyanocyclopropaneamido)imidazo[1,2-a]pyrimidin-2-yl]-4-fluorophenyl}-2,4-dimethyl-1,3-oxazole-5-carboxamide; N-{4-fluoro-3-[6-(oxane-3-amido)imidazo[1,2-a]pyrimidin-2-yl]phenyl}-2,4-dimethyl-1,3-oxazole-5-carboxamide; N-(4-fluoro-3-{6-[2-(oxan-2-yl)acetamido]imidazo[1,2-a]pyrimidin-2-yl}phenyl)-2,4-dimethyl-1,3-oxazole-5-carboxamide; N-{4-fluoro-3-[6-(5-oxooxolane-2-amido)imidazo[1,2-a]pyrimidin-2-yl]phenyl}-2,4-dimethyl-1,3-oxazole-5-carboxamide; N-(3-{6-[(1 S,2S,3R)-3-amino-7-oxabicyclo[2.2.1]heptane-2-amido]imidazo[1,2-a]pyrimidin-2-yl}-4-fluorophenyl)-2,4-dimethyl-1,3-oxazole-5-carboxamide; N-{4-fluoro-3-[6-(oxane-4-amido)imidazo[1,2-a]pyrimidin-2-yl]phenyl}-2,4-dimethyl-1,3-oxazole-5-carboxamide; N-{3-[6-(2,2-dimethyloxane-4-amido)imidazo[1,2-a]pyrimidin-2-yl]-4-fluorophenyl}-2,4-dimethyl-1,3-oxazole-5-carboxamide; N-(4-fluoro-3-{6-[(2S,3S)-3-hydroxypyrrolidine-2-amido]imidazo[1,2-a]pyrimidin-2-yl}phenyl)-2,4-dimethyl-1,3-oxazole-5-carboxamide; N-(4-fluoro-3-{6-[(2S)-oxolane-2-amido]imidazo[1,2-a]pyrimidin-2-yl}phenyl)-2,4-dimethyl-1,3-oxazole-5-carboxamide; N-{3-[6-(dimethyl-1,3-oxazole-4-amido)imidazo[1,2-a]pyrimidin-2-yl]-4-fluorophenyl}-2,4-dimethyl-1,3-oxazole-5-carboxamide; N-(4-fluoro-3-{6-[(2S)-3,3,3-trifluoro-2-hydroxy-2-methylpropanamido]imidazo[1,2-a]pyrimidin-2-yl}phenyl)-2,4-dimethyl-1,3-oxazole-5-carboxamide; N-(4-fluoro-3-{6-[(2R)-oxolane-2-amido]imidazo[1,2-a]pyrimidin-2-yl}phenyl)-2,4-dimethyl-1,3-oxazole-5-carboxamide; N-{2-[5-(dimethyl-1,3-oxazole-5-amido)-2-fluorophenyl]imidazo[1,2-a]pyrimidin-6-yl}-1-methylpiperidine-2-carboxamide; N-{4-fluoro-3-[6-(3-methoxypropanamido)imidazo[1,2-a]pyrimidin-2-yl]phenyl}-2,4-dimethyl-1,3-oxazole-5-carboxamide; N-{4-fluoro-3-[6-(1H-imidazole-4-amido)imidazo[1,2-a]pyrimidin-2-yl]phenyl}-2,4-dimethyl-1,3-oxazole-5-carboxamide; N-(3-{6-[(1R,2S)-2-aminocyclohexaneamido]imidazo[1,2-a]pyrimidin-2-yl}-4-fluorophenyl)-2,4-dimethyl-1,3-oxazole-5-carboxamide; N-{4-fluoro-3-[6-(4-methoxycyclohexaneamido)imidazo[1,2-a]pyrimidin-2-yl]phenyl}-2,4-dimethyl-1,3-oxazole-5-carboxamide; N-{4-fluoro-3-[6-(2-methyloxane-2-amido)imidazo[1,2-a]pyrimidin-2-yl]phenyl}-2,4-dimethyl-1,3-oxazole-5-carboxamide; N-{2-[5-(dimethyl-1,3-oxazole-5-amido)-2-fluorophenyl]imidazo[1,2-a]pyrimidin-6-yl}-3-methyl-1H-1,2,4-triazole-5-carboxamide; N-(4-fluoro-3-{6-[3-(propan-2-yl)-1H-pyrazole-5-amido]imidazo[1,2-a]pyrimidin-2-yl}phenyl)-2,4-dimethyl-1,3-oxazole-5-carboxamide; tert-butyl 2-({2-[5-(dimethyl-1,3-oxazole-5-amido)-2-fluorophenyl]imidazo[1,2-a]pyrimidin-6-yl}carbamoyl)-1,4-oxazepane-4-carboxylate; N-{4-fluoro-3-[6-(1-methyl-1H-imidazole-2-amido)imidazo[1,2-a]pyrimidin-2-yl]phenyl}-2,4-dimethyl-1,3-oxazole-5-carboxamide; N-[4-fluoro-3-(6-{4-[2-(4-methylpiperazin-1-yl)ethoxy]benzamido}imidazo[1,2-a]pyrimidin-2-yl)phenyl]-2,4-dimethyl-1,3-oxazole-5-carboxamide; tert-butyl N-[2-({2-[5-(dimethyl-1,3-oxazole-5-amido)-2-fluorophenyl]imidazo[1,2-a]pyrimidin-6-yl}carbamoyl)-1-methyl-1H-imidazol-4-yl]carbamate; N-{4-fluoro-3-[6-(2-methoxypropanamido)imidazo[1,2-a]pyrimidin-2-yl]phenyl}-2,4-dimethyl-1,3-oxazole-5-carboxamide; N-{3-[6-(4-amino-1-methyl-1H-imidazole-2-amido)imidazo[1,2-a]pyrimidin-2-yl]-4-fluorophenyl}-2,4-dimethyl-1,3-oxazole-5-carboxamide; N-{2-[5-(dimethyl-1,3-oxazole-5-amido)-2-fluorophenyl]imidazo[1,2-a]pyrimidin-6-yl}morpholine-2-carboxamide; N-(3-{6-[(1R,2S,3R,4S)-3-aminobicyclo[2.2.1]hept-5-ene-2-amido]imidazo[1,2-a]pyrimidin-2-yl}-4-fluorophenyl)-2,4-dimethyl-1,3-oxazole-5-carboxamide; N-(3-{6-[(1R,2R,3S,4S)-3-aminobicyclo[2.2.1]hept-5-ene-2-amido]imidazo[1,2-a]pyrimidin-2-yl}-4-fluorophenyl)-2,4-dimethyl-1,3-oxazole-5-carboxamide; N-{2-[5-(dimethyl-1,3-oxazole-5-amido)-2-fluorophenyl]imidazo[1,2-a]pyrimidin-6-yl}-1,4-oxazepane-2-carboxamide; N-{2-[5-(dimethyl-1,3-oxazole-5-amido)-2-fluorophenyl]imidazo[1,2-a]pyrimidin-6-yl}-5-methyl-1,2,4-oxadiazole-3-carboxamide; N-{4-fluoro-3-[6-(oxane-2-amido)imidazo[1,2-a]pyrimidin-2-yl]phenyl}-2,4-dimethyl-1,3-oxazole-5-carboxamide; N-{4-fluoro-3-[6-(oxane-2-amido)imidazo[1,2-a]pyrimidin-2-yl]phenyl}-2,4-dimethyl-1,3-oxazole-5-carboxamide; N-(3-{6-[2-(dimethylamino)acetamido]imidazo[1,2-a]pyrimidin-2-yl}-4-fluorophenyl)-2,4-dimethyl-1,3-oxazole-5-carboxamide; N-{2-[5-(dimethyl-1,3-oxazole-5-amido)-2-fluorophenyl]imidazo[1,2-a]pyrimidin-6-yl}-5-fluoropyridine-2-carboxamide; N-{4-fluoro-3-[6-(1,3-oxazole-2-amido)imidazo[1,2-a]pyrimidin-2-yl]phenyl}-2,4-dimethyl-1,3-oxazole-5-carboxamide; N-[3-(6-{4-[(dimethylamino)methyl]benzamido}imidazo[1,2-a]pyrimidin-2-yl)-4-fluorophenyl]-2,4-dimethyl-1,3-oxazole-5-carboxamide; N-{4-fluoro-3-[6-(2-methyl-1H-imidazole-5-amido)imidazo[1,2-a]pyrimidin-2-yl]phenyl}-2,4-dimethyl-1,3-oxazole-5-carboxamide; N-[4-fluoro-3-(6-{[3-(trifluoromethyl)pyridin-2-yl]amino}imidazo[1,2-a]pyrimidin-2-yl)phenyl]pyrrolidine-1-carboxamide; N-{4-fluoro-3-[6-(3-methyloxetane-3-amido)imidazo[1,2-a]pyrimidin-2-yl]phenyl}-2,4-dimethyl-1,3-oxazole-5-carboxamide; N-{4-fluoro-3-[6-(2-methyloxolane-2-amido)imidazo[1,2-a]pyrimidin-2-yl]phenyl}-2,4-dimethyl-1,3-oxazole-5-carboxamide; N-{2-[5-(dimethyl-1,3-oxazole-5-amido)-2-fluorophenyl]imidazo[1,2-a]pyrimidin-6-yl}-1-(propan-2-yl)piperidine-4-carboxamide; N-{2-[5-(dimethyl-1,3-oxazole-5-amido)-2-fluorophenyl]imidazo[1,2-a]pyrimidin-6-yl}-5-[(dimethylamino)methyl]pyridine-2-carboxamide; N-(3-{6-bromoimidazo[1,2-a]pyrimidin-2-yl}-4-fluorophenyl)cyclopentanecarboxamide; N-(3-{6-bromoimidazo[1,2-a]pyrimidin-2-yl}-4-fluorophenyl)benzamide; N-{4-fluoro-3-[6-(pyridin-2-yl)imidazo[1,2-a]pyrimidin-2-yl]phenyl}-2-methyl-1,3-oxazole-5-carboxamide; N-(4-fluoro-3-{6-fluoroimidazo[1,2-a]pyrimidin-2-yl}phenyl)-2,4-dimethyl-1,3-oxazole-5-carboxamide; N-{4-fluoro-3-[6-(trifluoromethyl)imidazo[1,2-a]pyrimidin-2-yl]phenyl}-2,4-dimethyl-1,3-oxazole-5-carboxamide; N-{4-fluoro-3-[6-(trifluoromethyl)imidazo[1,2-a]pyrimidin-2-yl]phenyl}-2-methyl-1,3-oxazole-5-carboxamide; propan-2-yl N-{2-[3-(2-methyl-1,3-oxazole-5-amido)phenyl]imidazo[1,2-a]pyrimidin-6-yl}carbamate; propan-2-yl N-{2-[3-(1,3-oxazole-5-amido)phenyl]imidazo[1,2-a]pyrimidin-6-yl}carbamate; propan-2-yl N-{2-[3-(4-methyl-1,3-oxazole-5-amido)phenyl]imidazo[1,2-a]pyrimidin-6-yl}carbamate; N-(3-{6-chloroimidazo[1,2-a]pyrimidin-2-yl}-4-fluorophenyl)-2,4-dimethyl-1,3-oxazole-5-carboxamide; N-(3-{6-acetamidoimidazo[1,2-a]pyrimidin-2-yl}-4-fluorophenyl)-2-methyl-1,3-oxazole-5-carboxamide; propan-2-yl N-({2-[2-fluoro-5-(5-fluorofuran-2-amido)phenyl]imidazo[1,2-a]pyrimidin-6-yl}methyl)carbamate; N-{3-[6-(difluoromethyl)imidazo[1,2-a]pyrimidin-2-yl]-4-fluorophenyl}-2,4-dimethyl-1,3-oxazole-5-carboxamide; N-{3-[6-(difluoromethyl)imidazo[1,2-a]pyrimidin-2-yl]-4-fluorophenyl}-2-methyl-1,3-oxazole-5-carboxamide; N-(3-{6-cyclobutylimidazo[1,2-a]pyrimidin-2-yl}-4-fluorophenyl)-2,4-dimethyl-1,3-oxazole-5-carboxamide; N-(3-{6-cyclobutylimidazo[1,2-a]pyrimidin-2-yl}-4-fluorophenyl)-2-methyl-1,3-oxazole-5-carboxamide; N-{4-fluoro-3-[6-(methylamino)imidazo[1,2-a]pyrimidin-2-yl]phenyl}-2,4-dimethyl-1,3-oxazole-5-carboxamide; N-(4-fluoro-3-{6-phenylimidazo[1,2-a]pyrimidin-2-yl}phenyl)cyclopentanecarboxamide; propan-2-yl N-[2-(2-chloro-5-{[methoxy(methyl)carbamoyl]amino}phenyl)imidazo[1,2-a]pyrimidin-6-yl]carbamate; propan-2-yl N-[2-(3-{[methoxy(methyl)carbamoyl]amino}phenyl)imidazo[1,2-a]pyrimidin-6-yl]carbamate; propan-2-yl N-(2-{2-chloro-5-[(morpholine-4-carbonyl)amino]phenyl}imidazo[1,2-a]pyrimidin-6-yl)carbamate; propan-2-yl N-(2-{2-chloro-5-[(2-oxoimidazolidine-1-carbonyl)amino]phenyl}imidazo[1,2-a]pyrimidin-6-yl)carbamate; propan-2-yl N-(2-{2-chloro-5-[(4-methylpiperazine-1-carbonyl)amino]phenyl}imidazo[1,2-a]pyrimidin-6-yl)carbamate; propan-2-yl N-(2-{5-[(dimethylcarbamoyl)amino]-2-fluorophenyl}imidazo[1,2-a]pyrimidin-6-yl)carbamate; propan-2-yl N-(2-{2-fluoro-5-[(methylcarbamoyl)amino]phenyl}imidazo[1,2-a]pyrimidin-6-yl)carbamate; propan-2-yl N-[2-(2-fluoro-5-{[methoxy(methyl)carbamoyl]amino}phenyl)imidazo[1,2-a]pyrimidin-6-yl]carbamate; propan-2-yl N-[2-(2-fluoro-5-{[methyl(2,2,2-trifluoroethyl)carbamoyl]amino}phenyl)imidazo[1,2-a]pyrimidin-6-yl]carbamate; N-{2-[5-(dimethyl-1,3-oxazole-5-amido)-2-fluorophenyl]imidazo[1,2-a]pyrimidin-6-yl}morpholine-4-carboxamide; N-(4-fluoro-3-{6-[(2-oxoimidazolidine-1-carbonyl)amino]imidazo[1,2-a]pyrimidin-2-yl}phenyl)-2,4-dimethyl-1,3-oxazole-5-carboxamide; 1-(3-{6-chloroimidazo[1,2-a]pyrimidin-2-yl}-4-fluorophenyl)-3,3-dimethylurea; propan-2-yl N-(2-{5-[(azetidine-1-carbonyl)amino]-2-fluorophenyl}imidazo[1,2-a]pyrimidin-6-yl)carbamate; propan-2-yl N-[2-(2-fluoro-5-{[(3S)-3-fluoropyrrolidine-1-carbonyl]amino}phenyl)imidazo[1,2-a]pyrimidin-6-yl]carbamate; propan-2-yl N-[2-(5-{[bis(propan-2-yl)carbamoyl]amino}-2-fluorophenyl)imidazo[1,2-a]pyrimidin-6-yl]carbamate; propan-2-yl N-[2-(2-fluoro-5-{[(3R)-3-fluoropyrrolidine-1-carbonyl]amino}phenyl)imidazo[1,2-a]pyrimidin-6-yl]carbamate; 1-(4-fluoro-3-{6-fluoroimidazo[1,2-a]pyrimidin-2-yl}phenyl)-3,3-dimethylurea; 3-fluoro-N-(4-fluoro-3-{6-fluoroimidazo[1,2-a]pyrimidin-2-yl}phenyl)azetidine-1-carboxamide; N-(4-fluoro-3-{6-fluoroimidazo[1,2-a]pyrimidin-2-yl}phenyl)azetidine-1-carboxamide; 3,3-difluoro-N-(4-fluoro-3-{6-fluoroimidazo[1,2-a]pyrimidin-2-yl}phenyl)azetidine-1-carboxamide; (3R)-3-fluoro-N-(4-fluoro-3-{6-fluoroimidazo[1,2-a]pyrimidin-2-yl}phenyl)pyrrolidine-1-carboxamide; N-(3-{6-chloroimidazo[1,2-a]pyrimidin-2-yl}-4-fluorophenyl)-3,3-difluoroazetidine-1-carboxamide; (3R)—N-(3-{6-chloroimidazo[1,2-a]pyrimidin-2-yl}-4-fluorophenyl)-3-fluoropyrrolidine-1-carboxamide; N-(3-{6-chloroimidazo[1,2-a]pyrimidin-2-yl}-4-fluorophenyl)azetidine-1-carboxamide; N-(3-{6-chloroimidazo[1,2-a]pyrimidin-2-yl}-4-fluorophenyl)-3-fluoroazetidine-1-carboxamide; 1-{3-[6-(dif luoromethyl)imidazo[1,2-a]pyrimidin-2-yl]-4-fluorophenyl}-3,3-dimethylurea; propan-2-yl N-[2-(5-cyclopropaneamido-2-fluorophenyl)imidazo[1,2-a]pyrimidin-6-yl]carbamate; propan-2-yl N-{2-[2-fluoro-5-(pyrazine-2-amido)phenyl]imidazo[1,2-a]pyrimidin-6-yl}carbamate; propan-2-yl N-{2-[2-fluoro-5-(1,3-thiazole-2-amido)phenyl]imidazo[1,2-a]pyrimidin-6-yl}carbamate; propan-2-yl N-{2-[2-chloro-5-(1,3-thiazole-2-amido)phenyl]imidazo[1,2-a]pyrimidin-6-yl}carbamate; propan-2-yl N-{2-[2-chloro-5-(furan-2-amido)phenyl]imidazo[1,2-a]pyrimidin-6-yl}carbamate; propan-2-yl N-[2-(2-chloro-5-cyclopropaneamidophenyl)imidazo[1,2-a]pyrimidin-6-yl]carbamate; propan-2-yl N-(2-{2-chloro-5-[(dimethylcarbamoyl)amino]phenyl}imidazo[1,2-a]pyrimidin-6-yl)carbamate; propan-2-yl N-[2-(2-chloro-5-propanamidophenyl)imidazo[1,2-a]pyrimidin-6-yl]carbamate; N-(3-{6-cyclopropaneamidoimidazo[1,2-a]pyrimidin-2-yl}-4-fluorophenyl)-2,4-dimethyl-1,3-oxazole-5-carboxamide; propan-2-yl N-[2-(2-chloro-5-{[methyl(2-methylpropyl)carbamoyl]amino}phenyl)imidazo[1,2-a]pyrimidin-6-yl]carbamate; propan-2-yl N-[2-(2-chloro-5-{[ethyl(methyl)carbamoyl]amino}phenyl)imidazo[1,2-a]pyrimidin-6-yl]carbamate; N-{4-fluoro-3-[6-(pyrazin-2-yl)imidazo[1,2-a]pyrimidin-2-yl]phenyl}-2,4-dimethyl-1,3-oxazole-5-carboxamide; propan-2-yl N-{2-[5-(carbamoylamino)-2-chlorophenyl]imidazo[1,2-a]pyrimidin-6-yl}carbamate; propan-2-yl N-[2-(2-chloro-5-{[(2-chloro-1,3-thiazol-5-yl)(methyl)carbamoyl]amino}phenyl)imidazo[1,2-a]pyrimidin-6-yl]carbamate; propan-2-yl N-(2-{2-chloro-5-[(ethylcarbamoyl)amino]phenyl}imidazo[1,2-a]pyrimidin-6-yl)carbamate; propan-2-yl N-[2-(2-chloro-5-{[methyl(2,2,2-trifluoroethyl)carbamoyl]amino}phenyl)imidazo[1,2-a]pyrimidin-6-yl]carbamate; propan-2-yl N-[2-(2-chloro-5-{[methyl(1-methyl-1H-imidazol-2-yl)carbamoyl]amino}phenyl)imidazo[1,2-a]pyrimidin-6-yl]carbamate; propan-2-yl N-(2-{2-chloro-5-[(hydrazinecarbonyl)amino]phenyl}imidazo[1,2-a]pyrimidin-6-yl)carbamate; propan-2-yl N-[2-(2-chloro-5-{[(1S,4S)-2-oxa-5-azabicyclo[2.2.1]heptane-5-carbonyl]amino}phenyl)imidazo[1,2-a]pyrimidin-6-yl]carbamate; propan-2-yl N-[2-(2-chloro-5-{[2-(pyridin-2-yl)pyrrolidine-1-carbonyl]amino}phenyl)imidazo[1,2-a]pyrimidin-6-yl]carbamate; propan-2-yl N-[2-(2-chloro-5-{[(2R)-2-(trifluoromethyl)pyrrolidine-1-carbonyl]amino}phenyl)imidazo[1,2-a]pyrimidin-6-yl]carbamate; propan-2-yl N-{2-[2-chloro-5-({1,1-difluoro-5-azaspiro[2.4]heptane-5-carbonyl}amino)phenyl]imidazo[1,2-a]pyrimidin-6-yl}carbamate; propan-2-yl N-[2-(5-{[(2S)-2-carbamoylpyrrolidine-1-carbonyl]amino}-2-chlorophenyl)imidazo[1,2-a]pyrimidin-6-yl]carbamate; propan-2-yl N-[2-(2-chloro-5-{[(2-methylpropyl)carbamoyl]amino}phenyl)imidazo[1,2-a]pyrimidin-6-yl]carbamate; propan-2-yl N-[2-(2-chloro-5-{[(3S)-3-fluoropyrrolidine-1-carbonyl]amino}phenyl)imidazo[1,2-a]pyrimidin-6-yl]carbamate; propan-2-yl N-[2-(2-chloro-5-{[(3R)-3-methoxypyrrolidine-1-carbonyl]amino}phenyl)imidazo[1,2-a]pyrimidin-6-yl]carbamate; propan-2-yl N-(2-{2-chloro-5-[(3,3,4,4-tetrafluoropyrrolidine-1-carbonyl)amino]phenyl}imidazo[1,2-a]pyrimidin-6-yl)carbamate; propan-2-yl N-[2-(2-chloro-5-{[(3R)-3-fluoropyrrolidine-1-carbonyl]amino}phenyl)imidazo[1,2-a]pyrimidin-6-yl]carbamate; propan-2-yl N-(2-{5-[(3-{[(tert-butoxy)carbonyl]amino}pyrrolidine-1-carbonyl)amino]-2-chlorophenyl}imidazo[1,2-a]pyrimidin-6-yl)carbamate; propan-2-yl N-(2-{2-chloro-5-[(cyclobutylcarbamoyl)amino]phenyl}imidazo[1,2-a]pyrimidin-6-yl)carbamate; propan-2-yl N-(2-{2-chloro-5-[(2,5-dimethylpyrrolidine-1-carbonyl)amino]phenyl}imidazo[1,2-a]pyrimidin-6-yl)carbamate; propan-2-yl N-[2-(2-chloro-5-{[(3R)-3-acetamidopyrrolidine-1-carbonyl]amino}phenyl)imidazo[1,2-a]pyrimidin-6-yl]carbamate; propan-2-yl N-(2-{2-chloro-5-[(2-methylpyrrolidine-1-carbonyl)amino]phenyl}imidazo[1,2-a]pyrimidin-6-yl)carbamate; propan-2-yl N-[2-(2-chloro-5-{[3-(morpholin-4-yl)pyrrolidine-1-carbonyl]amino}phenyl)imidazo[1,2-a]pyrimidin-6-yl]carbamate; propan-2-yl N-(2-{2-chloro-5-[(diethylcarbamoyl)amino]phenyl}imidazo[1,2-a]pyrimidin-6-yl)carbamate; propan-2-yl N-[2-(2-chloro-5-{[3-(dimethylamino)pyrrolidine-1-carbonyl]amino}phenyl)imidazo[1,2-a]pyrimidin-6-yl]carbamate; propan-2-yl N-[2-(2-chloro-5-{[3-(N-methylacetamido)pyrrolidine-1-carbonyl]amino}phenyl)imidazo[1,2-a]pyrimidin-6-yl]carbamate; propan-2-yl N-[2-(2-chloro-5-{[(propan-2-yl)carbamoyl]amino}phenyl)imidazo[1,2-a]pyrimidin-6-yl]carbamate; propan-2-yl N-(2-{5-[(azetidine-1-carbonyl)amino]-2-chlorophenyl}imidazo[1,2-a]pyrimidin-6-yl)carbamate; propan-2-yl N-(2-{2-chloro-5-[(3-oxoazetidine-1-carbonyl)amino]phenyl}imidazo[1,2-a]pyrimidin-6-yl)carbamate; 1-{[4-chloro-3-(6-{[(propan-2-yloxy)carbonyl]amino}imidazo[1,2-a]pyrimidin-2-yl)phenyl]carbamoyl}-3-methylpyrrolidine-3-carboxylic acid; propan-2-yl N-(2-{2-chloro-5-[(3-oxopyrrolidine-1-carbonyl)amino]phenyl}imidazo[1,2-a]pyrimidin-6-yl)carbamate; propan-2-yl N-(2-{2-chloro-5-[(2,2-dimethylpyrrolidine-1-carbonyl)amino]phenyl}imidazo[1,2-a]pyrimidin-6-yl)carbamate; propan-2-yl N-(2-{2-chloro-5-[(3-fluoroazetidine-1-carbonyl)amino]phenyl}imidazo[1,2-a]pyrimidin-6-yl)carbamate; propan-2-yl N-[2-(2-chloro-5-{[(3-methoxycyclobutyl)carbamoyl]amino}phenyl)imidazo[1,2-a]pyrimidin-6-yl]carbamate; propan-2-yl N-(2-{2-chloro-5-[(cyclopropylcarbamoyl)amino]phenyl}imidazo[1,2-a]pyrimidin-6-yl)carbamate; propan-2-yl N-[2-(2-chloro-5-{[(3R)-3-hydroxypyrrolidine-1-carbonyl]amino}phenyl)imidazo[1,2-a]pyrimidin-6-yl]carbamate; propan-2-yl N-(2-{2-fluoro-5-[(3-fluoroazetidine-1-carbonyl)amino]phenyl}imidazo[1,2-a]pyrimidin-6-yl)carbamate; propan-2-yl N-(2-{2-fluoro-5-[(3-hydroxyazetidine-1-carbonyl)amino]phenyl}imidazo[1,2-a]pyrimidin-6-yl)carbamate; propan-2-yl N-(2-{5-[(3,3-difluoroazetidine-1-carbonyl)amino]-2-fluorophenyl}imidazo[1,2-a]pyrimidin-6-yl)carbamate; propan-2-yl N-(2-{5-[(3-cyanoazetidine-1-carbonyl)amino]-2-fluorophenyl}imidazo[1,2-a]pyrimidin-6-yl)carbamate; propan-2-yl N-(2-{5-[(3-chloroazetidine-1-carbonyl)amino]-2-fluorophenyl}imidazo[1,2-a]pyrimidin-6-yl)carbamate; propan-2-yl N-(2-{2-fluoro-5-[(3-methanesulfonylazetidine-1-carbonyl)amino]phenyl}imidazo[1,2-a]pyrimidin-6-yl)carbamate; tert-butyl N-(1-{[4-fluoro-3-(6-{[(propan-2-yloxy)carbonyl]amino}imidazo[1,2-a]pyrimidin-2-yl)phenyl]carbamoyl}azetidin-3-yl)-N-methylcarbamate; propan-2-yl N-{2-[2-fluoro-5-({2-oxa-6-azaspiro[3.3]heptane-6-carbonyl}amino)phenyl]imidazo[1,2-a]pyrimidin-6-yl}carbamate; 1-{[4-fluoro-3-(6-{[(propan-2-yloxy)carbonyl]amino}imidazo[1,2-a]pyrimidin-2-yl)phenyl]carbamoyl}azetidine-2-carboxylic acid; 3,3-difluoro-N-{4-fluoro-3-[6-(pyridin-2-yl)imidazo[1,2-a]pyrimidin-2-yl]phenyl}azetidine-1-carboxamide; (3S)-3-fluoro-N-{4-fluoro-3-[6-(trifluoromethyl)imidazo[1,2-a]pyrimidin-2-yl]phenyl}pyrrolidine-1-carboxamide; 1-{4-fluoro-3-[6-(trifluoromethyl)imidazo[1,2-a]pyrimidin-2-yl]phenyl}-3,3-dimethylurea; N-{4-fluoro-3-[6-(trifluoromethyl)imidazo[1,2-a]pyrimidin-2-yl]phenyl}azetidine-1-carboxamide; 3,3-difluoro-N-{4-fluoro-3-[6-(trifluoromethyl)imidazo[1,2-a]pyrimidin-2-yl]phenyl}azetidine-1-carboxamide; propan-2-yl N-{4-fluoro-3-[6-(trifluoromethyl)imidazo[1,2-a]pyrimidin-2-yl]phenyl}carbamate; propan-2-yl N-[2-(2-fluoro-5-{[(propan-2-yloxy)carbonyl]amino}phenyl)imidazo[1,2-a]pyrimidin-6-yl]carbamate; propan-2-yl N-[2-(2-fluoro-5-{[(2-methoxyethoxy)carbonyl]amino}phenyl)imidazo[1,2-a]pyrimidin-6-yl]carbamate; 2-methoxyethyl N-{2-[5-(dimethyl-1,3-oxazole-5-amido)-2-fluorophenyl]imidazo[1,2-a]pyrimidin-6-yl}carbamate; ethyl N-{2-[5-(dimethyl-1,3-oxazole-5-amido)-2-fluorophenyl]imidazo[1,2-a]pyrimidin-6-yl}carbamate; cyclopentyl N-{2-[5-(dimethyl-1,3-oxazole-5-amido)-2-fluorophenyl]imidazo[1,2-a]pyrimidin-6-yl}carbamate; 2-fluoroethyl N-{2-[5-(dimethyl-1,3-oxazole-5-amido)-2-fluorophenyl]imidazo[1,2-a]pyrimidin-6-yl}carbamate; 2-methylpropyl N-{2-[5-(dimethyl-1,3-oxazole-5-amido)-2-fluorophenyl]imidazo[1,2-a]pyrimidin-6-yl}carbamate; butan-2-yl N-{2-[5-(dimethyl-1,3-oxazole-5-amido)-2-fluorophenyl]imidazo[1,2-a]pyrimidin-6-yl}carbamate; methyl N-{2-[5-(dimethyl-1,3-oxazole-5-amido)-2-fluorophenyl]imidazo[1,2-a]pyrimidin-6-yl}carbamate; prop-1-en-2-yl N-{2-[5-(dimethyl-1,3-oxazole-5-amido)-2-fluorophenyl]imidazo[1,2-a]pyrimidin-6-yl}carbamate; 1-methylcyclopropyl N-{2-[5-(dimethyl-1,3-oxazole-5-amido)-2-fluorophenyl]imidazo[1,2-a]pyrimidin-6-yl}carbamate; 2-methoxyethyl N-(2-{2-fluoro-5-[(pyrrolidine-1-carbonyl)amino]phenyl}imidazo[1,2-a]pyrimidin-6-yl)carbamate; propan-2-yl N-{4-fluoro-3-[6-(pyridin-2-yl)imidazo[1,2-a]pyrimidin-2-yl]phenyl}carbamate; N-{4-fluoro-3-[6-(propane-2-sulfonamido)imidazo[1,2-a]pyrimidin-2-yl]phenyl}-2,4-dimethyl-1,3-oxazole-5-carboxamide; N-(3-{6-bromoimidazo[1,2-a]pyrimidin-2-yl}-4-fluorophenyl)methanesulfonamide; 3-fluoro-N-(4-fluoro-3-{6-phenylimidazo[1,2-a]pyrimidin-2-yl}phenyl)azetidine-1-carboxamide; N-{3-[6-(3,6-dihydro-2H-pyran-4-yl)imidazo[1,2-a]pyrimidin-2-yl]-4-fluorophenyl}-3-fluoroazetidine-1-carboxamide; 3,3-difluoro-N-(4-fluoro-3-{6-phenylimidazo[1,2-a]pyrimidin-2-yl}phenyl)azetidine-1-carboxamide; N-{3-[6-(3,6-dihydro-2H-pyran-4-yl)imidazo[1,2-a]pyrimidin-2-yl]-4-fluorophenyl}-3,3-difluoroazetidine-1-carboxamide; N-(4-fluoro-3-{6-phenylimidazo[1,2-a]pyrimidin-2-yl}phenyl)azetidine-1-carboxamide; N-{3-[6-(3,6-dihydro-2H-pyran-4-yl)imidazo[1,2-a]pyrimidin-2-yl]-4-fluorophenyl}azetidine-1-carboxamide; 1-(4-fluoro-3-{6-phenylimidazo[1,2-a]pyrimidin-2-yl}phenyl)-3,3-dimethylurea; 1-{3-[6-(3,6-dihydro-2H-pyran-4-yl)imidazo[1,2-a]pyrimidin-2-yl]-4-fluorophenyl}-3,3-dimethylurea; 1-(4-fluoro-3-{6-phenylimidazo[1,2-a]pyrimidin-2-yl}phenyl)-3-methyl-3-(2,2,2-trifluoroethyl)urea; 1-{3-[6-(3,6-dihydro-2H-pyran-4-yl)imidazo[1,2-a]pyrimidin-2-yl]-4-fluorophenyl}-3-methyl-3-(2,2,2-trifluoroethyl)urea; 3-ethyl-1-(4-fluoro-3-{6-phenylimidazo[1,2-a]pyrimidin-2-yl}phenyl)-3-methylurea; 1-{3-[6-(3,6-dihydro-2H-pyran-4-yl)imidazo[1,2-a]pyrimidin-2-yl]-4-fluorophenyl}-3-ethyl-3-methylurea; N-(4-fluoro-3-{6-phenylimidazo[1,2-a]pyrimidin-2-yl}phenyl)-2-methyl-1,3-oxazole-5-carboxamide; N-{3-[6-(3,6-dihydro-2H-pyran-4-yl)imidazo[1,2-a]pyrimidin-2-yl]-4-fluorophenyl}-2-methyl-1,3-oxazole-5-carboxamide; propan-2-yl N-(4-fluoro-3-{6-phenylimidazo[1,2-a]pyrimidin-2-yl}phenyl)carbamate; propan-2-yl N-{3-[6-(3,6-dihydro-2H-pyran-4-yl)imidazo[1,2-a]pyrimidin-2-yl]-4-fluorophenyl}carbamate; N-(4-fluoro-3-{6-phenylimidazo[1,2-a]pyrimidin-2-yl}phenyl)-2,4-dimethyl-1,3-oxazole-5-carboxamide; N-{3-[6-(3,6-dihydro-2H-pyran-4-yl)imidazo[1,2-a]pyrimidin-2-yl]-4-fluorophenyl}-2,4-dimethyl-1,3-oxazole-5-carboxamide; N-{3-[6-(3,6-dihydro-2H-pyran-4-yl)imidazo[1,2-a]pyrimidin-2-yl]phenyl}-2,4-dimethyl-1,3-oxazole-5-carboxamide; 2,4-dimethyl-N-(3-{6-phenylimidazo[1,2-a]pyrimidin-2-yl}phenyl)-1,3-oxazole-5-carboxamide; (3R)-3-fluoro-N-(3-{6-phenylimidazo[1,2-a]pyrimidin-2-yl}phenyl)pyrrolidine-1-carboxamide; N-(4-fluoro-3-{3-fluoro-6-phenylimidazo[1,2-a]pyrimidin-2-yl}phenyl)-2,4-dimethyl-1,3-oxazole-5-carboxamide; N-(3-{6-cyclopropylimidazo[1,2-a]pyrimidin-2-yl}-4-fluorophenyl)-2,4-dimethyl-1,3-oxazole-5-carboxamide; propan-2-yl N-[4-(2-{2-fluoro-5-[(pyrrolidine-1-carbonyl)amino]phenyl}imidazo[1,2-a]pyrimidin-6-yl)phenyl]carbamate; N-(4-fluoro-3-{6-[2-(trifluoromethyl)pyridin-4-yl]imidazo[1,2-a]pyrimidin-2-yl}phenyl)pyrrolidine-1-carboxamide; N-{3-[6-(2-aminopyridin-4-yl)imidazo[1,2-a]pyrimidin-2-yl]-4-fluorophenyl}pyrrolidine-1-carboxamide; tert-butyl 4-(2-{2-chloro-5-[(pyrrolidine-1-carbonyl)amino]phenyl}imidazo[1,2-a]pyrimidin-6-yl)-1,2,3,6-tetrahydropyridine-1-carboxylate; N-{4-chloro-3-[6-(1-{[2-(trimethylsilyl)ethoxy]methyl}-1H-imidazol-4-yl)imidazo[1,2-a]pyrimidin-2-yl]phenyl}pyrrolidine-1-carboxamide; tert-butyl 4-[4-(2-{2-chloro-5-[(pyrrolidine-1-carbonyl)amino]phenyl}imidazo[1,2-a]pyrimidin-6-yl)phenoxy]piperidine-1-carboxylate; N-{4-fluoro-3-[6-(1-phenylethenyl)imidazo[1,2-a]pyrimidin-2-yl]phenyl}-2,4-dimethyl-1,3-oxazole-5-carboxamide; tert-butyl 4-{2-[5-(dimethyl-1,3-oxazole-5-amido)-2-fluorophenyl]imidazo[1,2-a]pyrimidin-6-yl}-1,2,3,6-tetrahydropyridine-1-carboxylate; N-[4-fluoro-3-(6-{4-[2-(morpholin-4-yl)ethyl]phenyl}imidazo[1,2-a]pyrimidin-2-yl)phenyl]-2,4-dimethyl-1,3-oxazole-5-carboxamide; N-(4-fluoro-3-{6-[4-(2-hydroxyethyl)phenyl]imidazo[1,2-a]pyrimidin-2-yl}phenyl)-2,4-dimethyl-1,3-oxazole-5-carboxamide; N-(4-fluoro-3-{6-[5-(2-hydroxyethyl)pyridin-2-yl]imidazo[1,2-a]pyrimidin-2-yl}phenyl)-2,4-dimethyl-1,3-oxazole-5-carboxamide; N-(4-fluoro-3-{6-[4-(methylamino)phenyl]imidazo[1,2-a]pyrimidin-2-yl}phenyl)pyrrolidine-1-carboxamide; N-[4-fluoro-3-(6-{4-[2-(morpholin-4-yl)ethyl]phenyl}imidazo[1,2-a]pyrimidin-2-yl)phenyl]pyrrolidine-1-carboxamide; N-{3-[6-(3,6-dihydro-2H-pyran-4-yl)imidazo[1,2-a]pyrimidin-2-yl]-4-fluorophenyl}pyrrolidine-1-carboxamide; N-{3-[6-(cyclopent-1-en-1-yl)imidazo[1,2-a]pyrimidin-2-yl]-4-fluorophenyl}pyrrolidine-1-carboxamide; N-{4-fluoro-3-[6-(1-methyl-1H-imidazol-4-yl)imidazo[1,2-a]pyrimidin-2-yl]phenyl}pyrrolidine-1-carboxamide; N-{4-fluoro-3-[6-(2-methylphenyl)imidazo[1,2-a]pyrimidin-2-yl]phenyl}pyrrolidine-1-carboxamide; N-{3-[6-(2-chlorophenyl)imidazo[1,2-a]pyrimidin-2-yl]-4-fluorophenyl}pyrrolidine-carboxamide; N-(4-fluoro-3-{6-phenylimidazo[1,2-a]pyrimidin-2-yl}phenyl)pyrrolidine-1-carboxamide; N-[4-fluoro-3-(6-{4-[2-(morpholin-4-yl)ethoxy]phenyl}imidazo[1,2-a]pyrimidin-2-yl)phenyl]-2,4-dimethyl-1,3-oxazole-5-carboxamide; N-[3-(6-{4-[(dimethylamino)methyl]phenyl}imidazo[1,2-a]pyrimidin-2-yl)-4-fluorophenyl]-2,4-dimethyl-1,3-oxazole-5-carboxamide; N-{4-fluoro-3-[6-(1-methyl-1,2,3,6-tetrahydropyridin-4-yl)imidazo[1,2-a]pyrimidin-2-yl]phenyl}-2,4-dimethyl-1,3-oxazole-5-carboxamide; N-{4-fluoro-3-[6-(2-fluoro-4-methanesulfonylphenyl)imidazo[1,2-a]pyrimidin-2-yl]phenyl}-2,4-dimethyl-1,3-oxazole-5-carboxamide; N-{4-fluoro-3-[6-(1-methyl-1H-pyrazol-3-yl)imidazo[1,2-a]pyrimidin-2-yl]phenyl}-2,4-dimethyl-1,3-oxazole-5-carboxamide; tert-butyl 2-{2-[5-(dimethyl-1,3-oxazole-5-amido)-2-fluorophenyl]imidazo[1,2-a]pyrimidin-6-yl}-1H-pyrrole-1-carboxylate; N-{3-[6-(dimethyl-1,3-thiazol-5-yl)imidazo[1,2-a]pyrimidin-2-yl]-4-fluorophenyl}-2,4-dimethyl-1,3-oxazole-5-carboxamide; N-{3-[6-(dimethyl-1,2-oxazol-4-yl)imidazo[1,2-a]pyrimidin-2-yl]-4-fluorophenyl}-2,4-dimethyl-1,3-oxazole-5-carboxamide; N-{4-fluoro-3-[6-(1,2-oxazol-4-yl)imidazo[1,2-a]pyrimidin-2-yl]phenyl}-2,4-dimethyl-1,3-oxazole-5-carboxamide; tert-butyl 4-(6-{2-[5-(dimethyl-1,3-oxazole-5-amido)-2-fluorophenyl]imidazo[1,2-a]pyrimidin-6-yl}pyridin-2-yl)piperazine-1-carboxylate; N-{4-fluoro-3-[6-(1H-pyrazol-5-yl)imidazo[1,2-a]pyrimidin-2-yl]phenyl}-2,4-dimethyl-1,3-oxazole-5-carboxamide; N-{4-fluoro-3-[6-(1-methyl-1H-imidazol-5-yl)imidazo[1,2-a]pyrimidin-2-yl]phenyl}-2,4-dimethyl-1,3-oxazole-5-carboxamide; N-{3-[6-(4,5-dihydrofuran-2-yl)imidazo[1,2-a]pyrimidin-2-yl]-4-fluorophenyl}-2,4-dimethyl-1,3-oxazole-5-carboxamide; N-{3-[6-(2-aminophenyl)imidazo[1,2-a]pyrimidin-2-yl]-4-fluorophenyl}-2,4-dimethyl-1,3-oxazole-5-carboxamide; N-{3-[6-(3,4-dihydro-2H-pyran-6-yl)imidazo[1,2-a]pyrimidin-2-yl]-4-fluorophenyl}-2,4-dimethyl-1,3-oxazole-5-carboxamide; N-{4-fluoro-3-[6-(1,3-thiazol-4-yl)imidazo[1,2-a]pyrimidin-2-yl]phenyl}-2,4-dimethyl-1,3-oxazole-5-carboxamide; N-{4-fluoro-3-[6-(1-methyl-1,2,5,6-tetrahydropyridin-3-yl)imidazo[1,2-a]pyrimidin-2-yl]phenyl}-2,4-dimethyl-1,3-oxazole-5-carboxamide; N-{4-fluoro-3-[6-(6-methylpyridin-2-yl)imidazo[1,2-a]pyrimidin-2-yl]phenyl}-2,4-dimethyl-1,3-oxazole-5-carboxamide; N-{4-fluoro-3-[6-(pyridin-4-yl)imidazo[1,2-a]pyrimidin-2-yl]phenyl}-2,4-dimethyl-1,3-oxazole-5-carboxamide; N-{3-[6-(1-benzyl-1,2,3,6-tetrahydropyridin-4-yl)imidazo[1,2-a]pyrimidin-2-yl]-4-fluorophenyl}-2,4-dimethyl-1,3-oxazole-5-carboxamide; N-{4-fluoro-3-[6-(2,2,6,6-tetramethyl-3,6-dihydro-2H-pyran-4-yl)imidazo[1,2-a]pyrimidin-2-yl]phenyl}-2,4-dimethyl-1,3-oxazole-5-carboxamide; N-[4-fluoro-3-(6-{4-[(propan-2-yl)amino]phenyl}imidazo[1,2-a]pyrimidin-2-yl)phenyl]-2,4-dimethyl-1,3-oxazole-5-carboxamide; 3-ethyl-1-{4-fluoro-3-[6-(pyridin-2-yl)imidazo[1,2-a]pyrimidin-2-yl]phenyl}-3-methylurea; N-[4-fluoro-3-(6-{4-[4-(propan-2-yl)piperazin-1-yl]phenyl}imidazo[1,2-a]pyrimidin-2-yl)phenyl]-2,4-dimethyl-1,3-oxazole-5-carboxamide; N-(4-fluoro-3-{6-[4-(morpholin-4-yl)phenyl]imidazo[1,2-a]pyrimidin-2-yl}phenyl)-2,4-dimethyl-1,3-oxazole-5-carboxamide; tert-butyl 4-(4-{2-[5-(dimethyl-1,3-oxazole-5-amido)-2-fluorophenyl]imidazo[, 2-a]pyrimidin-6-yl}-1H-pyrazol-1-yl)piperidine-1-carboxylate; N-(4-fluoro-3-{6-[1-(oxan-4-yl)-1H-pyrazol-4-yl]imidazo[1,2-a]pyrimidin-2-yl}phenyl)-2,4-dimethyl-1,3-oxazole-5-carboxamide; N-{4-fluoro-3-[6-(1H-pyrazol-4-yl)imidazo[1,2-a]pyrimidin-2-yl]phenyl}-2,4-dimethyl-1,3-oxazole-5-carboxamide; N-{3-[6-(1-ethyl-1H-pyrazol-5-yl)imidazo[1,2-a]pyrimidin-2-yl]-4-fluorophenyl}-2,4-dimethyl-1,3-oxazole-5-carboxamide; N-[4-fluoro-3-(6-{1-[2-(morpholin-4-yl)ethyl]-1H-pyrazol-4-yl}imidazo[1,2-a]pyrimidin-2-yl)phenyl]-2,4-dimethyl-1,3-oxazole-5-carboxamide; N-{4-fluoro-3-[6-(1-methyl-1H-pyrazol-4-yl)imidazo[1,2-a]pyrimidin-2-yl]phenyl}-2,4-dimethyl-1,3-oxazole-5-carboxamide; N-(4-fluoro-3-{6-[1-(propan-2-yl)-1H-pyrazol-5-yl]imidazo[1,2-a]pyrimidin-2-yl}phenyl)-2,4-dimethyl-1,3-oxazole-5-carboxamide; N-{4-fluoro-3-[6-(2-fluorophenyl)imidazo[1,2-a]pyrimidin-2-yl]phenyl}-2,4-dimethyl-1,3-oxazole-5-carboxamide; N-(4-fluoro-3-{6-[4-(methylamino)phenyl]imidazo[1,2-a]pyrimidin-2-yl}phenyl)-2,4-dimethyl-1,3-oxazole-5-carboxamide; N-{4-fluoro-3-[6-(1-methyl-1H-pyrazol-5-yl)imidazo[1,2-a]pyrimidin-2-yl]phenyl}-2,4-dimethyl-1,3-oxazole-5-carboxamide; N-{4-fluoro-3-[6-(pyridin-2-yl)imidazo[1,2-a]pyrimidin-2-yl]phenyl}-2,4-dimethyl-1,3-oxazole-5-carboxamide; N-{4-fluoro-3-[6-(pyridin-3-yl)imidazo[1,2-a]pyrimidin-2-yl]phenyl}-2,4-dimethyl-1,3-oxazole-5-carboxamide; N-{4-fluoro-3-[6-(4-fluorophenyl)imidazo[1,2-a]pyrimidin-2-yl]phenyl}-2,4-dimethyl-1,3-oxazole-5-carboxamide; N-{4-fluoro-3-[6-(2-methanesulfonylphenyl)imidazo[1,2-a]pyrimidin-2-yl]phenyl}-2,4-dimethyl-1,3-oxazole-5-carboxamide; N-{4-fluoro-3-[6-(pyridin-3-yl)imidazo[1,2-a]pyrimidin-2-yl]phenyl}-2-methyl-1,3-oxazole-5-carboxamide; propan-2-yl N-{4-fluoro-3-[6-(pyridin-3-yl)imidazo[1,2-a]pyrimidin-2-yl]phenyl}carbamate; 3-fluoro-N-{4-fluoro-3-[6-(pyridin-2-yl)imidazo[1,2-a]pyrimidin-2-yl]phenyl}azetidine-1-carboxamide; 3-fluoro-N-{4-fluoro-3-[6-(pyridin-3-yl)imidazo[1,2-a]pyrimidin-2-yl]phenyl}azetidine-1-carboxamide; 3,3-difluoro-N-{4-fluoro-3-[6-(pyridin-3-yl)imidazo[1,2-a]pyrimidin-2-yl]phenyl}azetidine-1-carboxamide; 3,3-difluoro-N-{4-fluoro-3-[6-(3-methylpyridin-2-yl)imidazo[1,2-a]pyrimidin-2-yl]phenyl}azetidine-1-carboxamide; N-{4-fluoro-3-[6-(pyridin-2-yl)imidazo[1,2-a]pyrimidin-2-yl]phenyl}azetidine-1-carboxamide; N-{4-fluoro-3-[6-(pyridin-3-yl)imidazo[1,2-a]pyrimidin-2-yl]phenyl}azetidine-1-carboxamide; 1-{4-fluoro-3-[6-(pyridin-2-yl)imidazo[1,2-a]pyrimidin-2-yl]phenyl}-3,3-dimethylurea; (3R)-3-fluoro-N-{4-fluoro-3-[6-(pyridin-2-yl)imidazo[1,2-a]pyrimidin-2-yl]phenyl}pyrrolidine-1-carboxamide; (3R)-3-fluoro-N-{4-fluoro-3-[6-(pyridin-3-yl)imidazo[1,2-a]pyrimidin-2-yl]phenyl}pyrrolidine-1-carboxamide; (3R)-3-fluoro-N-(4-fluoro-3-{6-phenylimidazo[1,2-a]pyrimidin-2-yl}phenyl)pyrrolidine-1-carboxamide; (3R)-3-fluoro-N-{4-fluoro-3-[6-(3-methylpyridin-2-yl)imidazo[1,2-a]pyrimidin-2-yl]phenyl}pyrrolidine-1-carboxamide; N-{4-fluoro-3-[6-(propan-2-yl)imidazo[1,2-a]pyrimidin-2-yl]phenyl}azetidine-1-carboxamide; N-{4-fluoro-3-[6-(propan-2-yl)imidazo[1,2-a]pyrimidin-2-yl]phenyl}-2,4-dimethyl-1,3-oxazole-5-carboxamide; N-{4-fluoro-3-[6-(propan-2-yl)imidazo[1,2-a]pyrimidin-2-yl]phenyl}-2-methyl-1,3-oxazole-5-carboxamide; propan-2-yl N-{4-fluoro-3-[6-(propan-2-yl)imidazo[1,2-a]pyrimidin-2-yl]phenyl}carbamate; 3-fluoro-N-{4-fluoro-3-[6-(propan-2-yl)imidazo[1,2-a]pyrimidin-2-yl]phenyl}azetidine-1-carboxamide; 3,3-difluoro-N-{4-fluoro-3-[6-(propan-2-yl)imidazo[1,2-a]pyrimidin-2-yl]phenyl}azetidine-1-carboxamide; 1-{4-fluoro-3-[6-(propan-2-yl)imidazo[1,2-a]pyrimidin-2-yl]phenyl}-3,3-dimethylurea; (3R)-3-fluoro-N-{4-fluoro-3-[6-(propan-2-yl)imidazo[1,2-a]pyrimidin-2-yl]phenyl}pyrrolidine-1-carboxamide; N-{4-fluoro-3-[6-(oxan-4-yl)imidazo[1,2-a]pyrimidin-2-yl]phenyl}pyrrolidine-1-carboxamide; N-{4-fluoro-3-[6-(1-phenylethyl)imidazo[1,2-a]pyrimidin-2-yl]phenyl}-2,4-dimethyl-1,3-oxazole-5-carboxamide; N-{4-fluoro-3-[6-(oxan-4-yl)imidazo[1,2-a]pyrimidin-2-yl]phenyl}-2,4-dimethyl-1,3-oxazole-5-carboxamide; N-(3-{6-cyclopentylimidazo[1,2-a]pyrimidin-2-yl}-4-fluorophenyl)-2,4-dimethyl-1,3-oxazole-5-carboxamide; N-(3-{6-ethylimidazo[1,2-a]pyrimidin-2-yl}-4-fluorophenyl)-2,4-dimethyl-1,3-oxazole-5-carboxamide; N-{4-fluoro-3-[6-(oxan-4-yl)imidazo[1,2-a]pyrimidin-2-yl]phenyl}-2-methyl-1,3-oxazole-5-carboxamide; tert-butyl 4-{2-[5-(dimethyl-1,3-oxazole-5-amido)-2-fluorophenyl]imidazo[1,2-a]pyrimidin-6-yl}piperidine-1-carboxylate; N-{4-fluoro-3-[6-(1-methyl-1H-imidazol-4-yl)imidazo[1,2-a]pyrimidin-2-yl]phenyl}-2,4-dimethyl-1,3-oxazole-5-carboxamide; N-{4-fluoro-3-[6-(1-methyl-1H-imidazol-2-yl)imidazo[1,2-a]pyrimidin-2-yl]phenyl}-2,4-dimethyl-1,3-oxazole-5-carboxamide; N-(4-fluoro-3-{6-[1-(propan-2-yl)-1H-imidazol-4-yl]imidazo[1,2-a]pyrimidin-2-yl}phenyl)-2,4-dimethyl-1,3-oxazole-5-carboxamide; N-{4-fluoro-3-[6-(1H-imidazol-4-yl)imidazo[1,2-a]pyrimidin-2-yl]phenyl}-2,4-dimethyl-1,3-oxazole-5-carboxamide; N-{4-fluoro-3-[6-(5-fluoro-3-methylpyridin-2-yl)imidazo[1,2-a]pyrimidin-2-yl]phenyl}-2,4-dimethyl-1,3-oxazole-5-carboxamide; N-{4-fluoro-3-[6-(5-fluoropyridin-2-yl)imidazo[1,2-a]pyrimidin-2-yl]phenyl}-2,4-dimethyl-1,3-oxazole-5-carboxamide; N-(4-fluoro-3-{6-[5-(methylamino)pyridin-2-yl]imidazo[1,2-a]pyrimidin-2-yl}phenyl)-2,4-dimethyl-1,3-oxazole-5-carboxamide; 6-{2-[5-(dimethyl-1,3-oxazole-5-amido)-2-fluorophenyl]imidazo[1,2-a]pyrimidin-6-yl}pyridine-2-carboxamide; N-{4-fluoro-3-[6-(6-fluoropyridin-2-yl)imidazo[1,2-a]pyrimidin-2-yl]phenyl}-2,4-dimethyl-1,3-oxazole-5-carboxamide; N-{4-fluoro-3-[6-(4-fluoropyridin-2-yl)imidazo[1,2-a]pyrimidin-2-yl]phenyl}-2,4-dimethyl-1,3-oxazole-5-carboxamide; N-{4-fluoro-3-[6-(6-fluoropyridin-3-yl)imidazo[1,2-a]pyrimidin-2-yl]phenyl}-2,4-dimethyl-1,3-oxazole-5-carboxamide; N-{3-[6-(6-acetylpyridin-3-yl)imidazo[1,2-a]pyrimidin-2-yl]-4-fluorophenyl}-2,4-dimethyl-1,3-oxazole-5-carboxamide; N-(4-fluoro-3-{6-[5-(propan-2-yl)pyridin-3-yl]imidazo[1,2-a]pyrimidin-2-yl}phenyl)-2,4-dimethyl-1,3-oxazole-5-carboxamide; N-{4-fluoro-3-[6-(2-fluoro-5-methylpyridin-3-yl)imidazo[1,2-a]pyrimidin-2-yl]phenyl}-2,4-dimethyl-1,3-oxazole-5-carboxamide; N-{4-fluoro-3-[6-(5-methylpyridin-3-yl)imidazo[1,2-a]pyrimidin-2-yl]phenyl}-2,4-dimethyl-1,3-oxazole-5-carboxamide; N-{4-fluoro-3-[6-(6-methylpyridin-3-yl)imidazo[1,2-a]pyrimidin-2-yl]phenyl}-2,4-dimethyl-1,3-oxazole-5-carboxamide; N-{4-fluoro-3-[6-(4-methylpyridin-3-yl)imidazo[1,2-a]pyrimidin-2-yl]phenyl}-2,4-dimethyl-1,3-oxazole-5-carboxamide; N-{4-fluoro-3-[6-(5-fluoropyridin-3-yl)imidazo[1,2-a]pyrimidin-2-yl]phenyl}-2,4-dimethyl-1,3-oxazole-5-carboxamide; N-{4-fluoro-3-[6-(3-fluoropyridin-4-yl)imidazo[1,2-a]pyrimidin-2-yl]phenyl}-2,4-dimethyl-1,3-oxazole-5-carboxamide; N-{4-fluoro-3-[6-(5-methylpyridin-2-yl)imidazo[1,2-a]pyrimidin-2-yl]phenyl}-2,4-dimethyl-1,3-oxazole-5-carboxamide; N-(4-fluoro-3-{6-[5-(propan-2-yl)pyrazin-2-yl]imidazo[1,2-a]pyrimidin-2-yl}phenyl)-2,4-dimethyl-1,3-oxazole-5-carboxamide; N-{3-[6-(3,5-difluoropyridin-2-yl)imidazo[1,2-a]pyrimidin-2-yl]-4-fluorophenyl}-2,4-dimethyl-1,3-oxazole-5-carboxamide; N-{4-fluoro-3-[6-(6-methylpyridazin-3-yl)imidazo[1,2-a]pyrimidin-2-yl]phenyl}-2,4-dimethyl-1,3-oxazole-5-carboxamide; N-{4-fluoro-3-[6-(3-fluoropyridin-2-yl)imidazo[1,2-a]pyrimidin-2-yl]phenyl}-2,4-dimethyl-1,3-oxazole-5-carboxamide; N-[3-(6-{4-[2-(dimethylamino)ethyl]phenyl}imidazo[1,2-a]pyrimidin-2-yl)-4-fluorophenyl]-2,4-dimethyl-1,3-oxazole-5-carboxamide; N-[4-fluoro-3-(6-{4-[2-(methylamino)ethyl]phenyl}imidazo[1,2-a]pyrimidin-2-yl)phenyl]-2,4-dimethyl-1,3-oxazole-5-carboxamide; N-{4-fluoro-3-[6-(5-methylpyrazin-2-yl)imidazo[1,2-a]pyrimidin-2-yl]phenyl}-2,4-dimethyl-1,3-oxazole-5-carboxamide; N-{4-fluoro-3-[6-(3-methylpyridin-2-yl)imidazo[1,2-a]pyrimidin-2-yl]phenyl}-2,4-dimethyl-1,3-oxazole-5-carboxamide; N-{3-[6-(3-chloropyridin-2-yl)imidazo[1,2-a]pyrimidin-2-yl]-4-fluorophenyl}-2,4-dimethyl-1,3-oxazole-5-carboxamide; N-{4-fluoro-3-[6-(pyrazin-2-yl)imidazo[1,2-a]pyrimidin-2-yl]phenyl}-2,4-dimethyl-1,3-oxazole-5-carboxamide; N-{4-fluoro-3-[6-(6-methylpyridin-2-yl)imidazo[1,2-a]pyrimidin-2-yl]phenyl}pyrrolidine-1-carboxamide; N-{4-chloro-3-[6-(pyridin-2-yl)imidazo[1,2-a]pyrimidin-2-yl]phenyl}pyrrolidine-1-carboxamide; N-{3-[6-(dimethyl-1,3-thiazol-2-yl)imidazo[1,2-a]pyrimidin-2-yl]-4-fluorophenyl}-2,4-dimethyl-1,3-oxazole-5-carboxamide; N-{4-fluoro-3-[6-(6-methoxypyridin-2-yl)imidazo[1,2-a]pyrimidin-2-yl]phenyl}-2,4-dimethyl-1,3-oxazole-5-carboxamide; N-{4-fluoro-3-[6-(3-methoxypyridin-2-yl)imidazo[1,2-a]pyrimidin-2-yl]phenyl}-2,4-dimethyl-1,3-oxazole-5-carboxamide; 3,3-difluoro-N-(4-fluoro-3-{6-[(propan-2-yl)amino]imidazo[1,2-a]pyrimidin-2-yl}phenyl)azetidine-1-carboxamide; (3R)-3-fluoro-N-(4-fluoro-3-{6-[(propan-2-yl)amino]imidazo[1,2-a]pyrimidin-2-yl}phenyl)pyrrolidine-1-carboxamide; N-(4-fluoro-3-{6-[(propan-2-yl)amino]imidazo[1,2-a]pyrimidin-2-yl}phenyl)-2-methyl-1,3-oxazole-5-carboxamide; N-(4-fluoro-3-{6-[(2-methylpropyl)amino]imidazo[1,2-a]pyrimidin-2-yl}phenyl)-2,4-dimethyl-1,3-oxazole-5-carboxamide; N-(3-{6-[(butan-2-yl)amino]imidazo[1,2-a]pyrimidin-2-yl}-4-fluorophenyl)-2,4-dimethyl-1,3-oxazole-5-carboxamide; N-(3-{6-[(2,2-dimethylpropyl)amino]imidazo[1,2-a]pyrimidin-2-yl}-4-fluorophenyl)-2,4-dimethyl-1,3-oxazole-5-carboxamide; N-{3-[6-(dimethylamino)imidazo[1,2-a]pyrimidin-2-yl]-4-fluorophenyl}-3,3-difluoroazetidine-1-carboxamide; N-{3-[6-(dimethylamino)imidazo[1,2-a]pyrimidin-2-yl]-4-fluorophenyl}-2-methyl-1,3-oxazole-5-carboxamide; (3R)—N-{3-[6-(dimethylamino)imidazo[1,2-a]pyrimidin-2-yl]-4-fluorophenyl}-3-fluoropyrrolidine-1-carboxamide; N-{3-[6-(dimethylamino)imidazo[1,2-a]pyrimidin-2-yl]-4-fluorophenyl}azetidine-1-carboxamide; N-{3-[6-(dimethylamino)imidazo[1,2-a]pyrimidin-2-yl]-4-fluorophenyl}-2,4-dimethyl-1,3-oxazole-5-carboxamide; N-{3-[6-(diethylamino)imidazo[1,2-a]pyrimidin-2-yl]-4-fluorophenyl}-2,4-dimethyl-1,3-oxazole-5-carboxamide; N-(4-fluoro-3-{6-[(2-methylpropyl)amino]imidazo[1,2-a]pyrimidin-2-yl}phenyl)pyrrolidine-1-carboxamide; tert-butyl N-[(1 s,4s)-4-{[(2-{2-fluoro-5-[(pyrrolidine-1-carbonyl)amino]phenyl}imidazo[1,2-a]pyrimidin-6-yl)amino]methyl}cyclohexyl]carbamate; N-{4-fluoro-3-[6-(propylamino)imidazo[1,2-a]pyrimidin-2-yl]phenyl}pyrrolidine-1-carboxamide; N-(4-fluoro-3-{6-[(oxan-4-yl)amino]imidazo[1,2-a]pyrimidin-2-yl}phenyl)pyrrolidine-1-carboxamide; N-(4-fluoro-3-{6-[(propan-2-yl)amino]imidazo[1,2-a]pyrimidin-2-yl}phenyl)pyrrolidine-1-carboxamide; tert-butyl N-[(1 r,4r)-4-{[(2-{2-fluoro-5-[(pyrrolidine-1-carbonyl)amino]phenyl}imidazo[1,2-a]pyrimidin-6-yl)amino]methyl}cyclohexyl]carbamate; N-(4-fluoro-3-{6-[(3,3,3-trifluoropropyl)amino]imidazo[1,2-a]pyrimidin-2-yl}phenyl)pyrrolidine-1-carboxamide; N-(4-fluoro-3-{6-[(1H-imidazol-2-ylmethyl)amino]imidazo[1,2-a]pyrimidin-2-yl}phenyl)-2,4-dimethyl-1,3-oxazole-5-carboxamide; N-(4-fluoro-3-{6-[(1H-imidazol-4-ylmethyl)amino]imidazo[1,2-a]pyrimidin-2-yl}phenyl)-2,4-dimethyl-1,3-oxazole-5-carboxamide; N-(4-fluoro-3-{6-[(propan-2-yl)amino]imidazo[1,2-a]pyrimidin-2-yl}phenyl)-2,4-dimethyl-1,3-oxazole-5-carboxamide; N-{3-[6-(cyclobutylamino)imidazo[1,2-a]pyrimidin-2-yl]-4-fluorophenyl}-2,4-dimethyl-1,3-oxazole-5-carboxamide; N-(4-fluoro-3-{6-[methyl(propan-2-yl)amino]imidazo[1,2-a]pyrimidin-2-yl}phenyl)-2,4-dimethyl-1,3-oxazole-5-carboxamide; propan-2-yl N-(2-{2-fluoro-5-[(pyridin-2-yl)amino]phenyl}imidazo[1,2-a]pyrimidin-6-yl)carbamate; propan-2-yl N-[2-(2-fluoro-5-{[3-(trifluoromethyl)pyridin-2-yl]amino}phenyl)imidazo[1,2-a]pyrimidin-6-yl]carbamate; propan-2-yl N-(2-{2-fluoro-5-[(pyridin-3-yl)amino]phenyl}imidazo[1,2-a]pyrimidin-6-yl)carbamate; propan-2-yl N-[2-(2-fluoro-5-{[3-(trifluoromethoxy)pyridin-2-yl]amino}phenyl)imidazo[1,2-a]pyrimidin-6-yl]carbamate; N-[4-fluoro-3-(6-{[3-(trifluoromethyl)pyridin-2-yl]amino}imidazo[1,2-a]pyrimidin-2-yl)phenyl]-2,4-dimethyl-1,3-oxazole-5-carboxamide; N-{4-chloro-3-[6-(3-hydroxy-3-methylazetidin-1-yl)imidazo[1,2-a]pyrimidin-2-yl]phenyl}pyrrolidine-1-carboxamide; N-{4-fluoro-3-[6-(morpholin-4-yl)imidazo[1,2-a]pyrimidin-2-yl]phenyl}-2,4-dimethyl-1,3-oxazole-5-carboxamide; N-(3-{6-[(2R,6S)-2,6-dimethylmorpholin-4-yl]imidazo[1,2-a]pyrimidin-2-yl}-4-fluorophenyl)-2,4-dimethyl-1,3-oxazole-5-carboxamide; N-(4-fluoro-3-{6-[(2R)-2-(trifluoromethyl)pyrrolidin-1-yl]imidazo[1,2-a]pyrimidin-2-yl}phenyl)-2,4-dimethyl-1,3-oxazole-5-carboxamide; N-{4-fluoro-3-[6-(pyrrolidin-1-yl)imidazo[1,2-a]pyrimidin-2-yl]phenyl}-2,4-dimethyl-1,3-oxazole-5-carboxamide; N-{3-[6-(cyclopropylamino)imidazo[1,2-a]pyrimidin-2-yl]-4-fluorophenyl}-2,4-dimethyl-1,3-oxazole-5-carboxamide; N-{4-chloro-3-[6-(pyrrolidin-1-yl)imidazo[1,2-a]pyrimidin-2-yl]phenyl}pyrrolidine-1-carboxamide; N-(4-chloro-3-{6-[(2-methoxyethyl)amino]imidazo[1,2-a]pyrimidin-2-yl}phenyl)pyrrolidine-1-carboxamide; propan-2-yl N-{2-[2-fluoro-5-({furo[2,3-c]pyridin-7-yl}amino)phenyl]imidazo[1,2-a]pyrimidin-6-yl}carbamate; propan-2-yl N-(2-{2-fluoro-5-[(3-methoxypyridin-2-yl)amino]phenyl}imidazo[1,2-a]pyrimidin-6-yl)carbamate; propan-2-yl N-(2-{5-[(4,5-dihydro-1,3-oxazol-2-yl)amino]-2-fluorophenyl}imidazo[1,2-a]pyrimidin-6-yl)carbamate; propan-2-yl N-(2-{5-[(4,4-dimethyl-4,5-dihydro-1,3-oxazol-2-yl)amino]-2-fluorophenyl}imidazo[1,2-a]pyrimidin-6-yl)carbamate; propan-2-yl N-(2-{2-fluoro-5-[(5-methyl-4,5-dihydro-1,3-oxazol-2-yl)amino]phenyl}imidazo[1,2-a]pyrimidin-6-yl)carbamate; propan-2-yl N-(2-{2-fluoro-5-[(4-methyl-4,5-dihydro-1,3-oxazol-2-yl)amino]phenyl}imidazo[1,2-a]pyrimidin-6-yl)carbamate; propan-2-yl N-(2-{5-[(5,5-dimethyl-4,5-dihydro-1,3-oxazol-2-yl)amino]-2-fluorophenyl}imidazo[1,2-a]pyrimidin-6-yl)carbamate; N-(4-fluoro-3-{6-[(5-methyl-4,5-dihydro-1,3-oxazol-2-yl)amino]imidazo[1,2-a]pyrimidin-2-yl}phenyl)-2,4-dimethyl-1,3-oxazole-5-carboxamide; N-(3-{6-[(4,4-dimethyl-4,5-dihydro-1,3-oxazol-2-yl)amino]imidazo[1,2-a]pyrimidin-2-yl}-4-fluorophenyl)-2,4-dimethyl-1,3-oxazole-5-carboxamide; N-(4-fluoro-3-{6-[(1-methyl-4,5-dihydro-1H-imidazol-2-yl)amino]imidazo[1,2-a]pyrimidin-2-yl}phenyl)-2,4-dimethyl-1,3-oxazole-5-carboxamide; N-(3-{6-[(5,5-dimethyl-4,5-dihydro-1,3-oxazol-2-yl)amino]imidazo[1,2-a]pyrimidin-2-yl}-4-fluorophenyl)-2,4-dimethyl-1,3-oxazole-5-carboxamide; N-[4-fluoro-3-(6-{[5-(trifluoromethyl)-4,5-dihydro-1,3-oxazol-2-yl]amino}imidazo[1,2-a]pyrimidin-2-yl)phenyl]-2,4-dimethyl-1,3-oxazole-5-carboxamide; N-(3-{6-[(5,5-dimethyl-4,5-dihydro-1H-imidazol-2-yl)amino]imidazo[1,2-a]pyrimidin-2-yl}-4-fluorophenyl)-2,4-dimethyl-1,3-oxazole-5-carboxamide; N-[3-(6-{[(3aS,7aS)-3a,4,5,6,7,7a-hexahydro-1,3-benzoxazol-2-yl]amino}imidazo[1,2-a]pyrimidin-2-yl)-4-fluorophenyl]-2,4-dimethyl-1,3-oxazole-5-carboxamide; N-[3-(6-{[(3aR,6aR)-3aH,4H,5H,6H,6aH-cyclopenta[d][1,3]oxazol-2-yl]amino}imidazo[1,2-a]pyrimidin-2-yl)-4-fluorophenyl]-2,4-dimethyl-1,3-oxazole-5-carboxamide; benzyl N-(2-{2-fluoro-5-[(pyrrolidine-1-carbonyl)amino]phenyl}imidazo[1,2-a]pyrimidin-6-yl)carbamate; benzyl N-(2-{4-fluoro-3-[(pyrrolidine-1-carbonyl)amino]phenyl}imidazo[1,2-a]pyrimidin-6-yl)carbamate; N-(4-fluoro-3-{6-fluoroimidazo[1,2-a]pyrimidin-2-yl}phenyl)pyrrolidine-1-carboxamide; N-(3-{6-bromoimidazo[1,2-a]pyrimidin-2-yl}-4-fluorophenyl)morpholine-4-carboxamide; N-(3-{6-bromoimidazo[1,2-a]pyrimidin-2-yl}-4-fluorophenyl)-4-methylpiperazine-1-carboxamide; methyl N-(3-{6-bromoimidazo[1,2-a]pyrimidin-2-yl}-4-fluorophenyl)carbamate; benzyl N-[2-(2-fluoro-5-{[(propan-2-yloxy)carbonyl]amino}phenyl)imidazo[1,2-a]pyrimidin-6-yl]carbamate; 3-chloropropyl N-{2-[5-(dimethyl-1,3-oxazole-5-amido)-2-fluorophenyl]imidazo[1,2-a]pyrimidin-6-yl}carbamate; N-{4-chloro-3-[6-(2-oxo-1,3-oxazolidin-3-yl)imidazo[1,2-a]pyrimidin-2-yl]phenyl}-2,4-dimethyl-1,3-oxazole-5-carboxamide; N-(4-fluoro-3-{6-[(hydrazinecarbonyl)amino]imidazo[1,2-a]pyrimidin-2-yl}phenyl)pyrrolidine-1-carboxamide; N-[4-fluoro-3-(6-{[N′-(propan-2-yl)hydrazinecarbonyl]amino}imidazo[1,2-a]pyrimidin-2-yl)phenyl]pyrrolidine-1-carboxamide; propan-2-yl N-(2-{2-fluoro-5-[(propan-2-yl)amino]phenyl}imidazo[1,2-a]pyrimidin-6-yl)carbamate; N-(4-fluoro-3-{6-[(4-methyl-4,5-dihydro-1,3-oxazol-2-yl)amino]imidazo[1,2-a]pyrimidin-2-yl}phenyl)-2,4-dimethyl-1,3-oxazole-5-carboxamide; N-(3-{6-acetamidoimidazo[1,2-a]pyrimidin-2-yl}-4-fluorophenyl)-2,4-dimethyl-1,3-oxazole-5-carboxamide; propan-2-yl N-{2-[3-(5-methyl-1,3,4-oxadiazole-2-amido)phenyl]imidazo[1,2-a]pyrimidin-6-yl}carbamate; propan-2-yl N-{2-[2-chloro-5-(5-methyl-1,3,4-oxadiazole-2-amido)phenyl]imidazo[1,2-a]pyrimidin-6-yl}carbamate; propan-2-yl N-{2-[2-chloro-5-(5-methyl-1,2,4-oxadiazole-3-amido)phenyl]imidazo[1,2-a]pyrimidin-6-yl}carbamate; tert-butyl N-[2-(5-acetamido-2-fluorophenyl)imidazo[1,2-a]pyrimidin-6-yl]carbamate; propan-2-yl N-{2-[2-chloro-5-(1,3-dioxo-2,3-dihydro-1H-isoindol-2-yl)phenyl]imidazo[1,2-a]pyrimidin-6-yl}carbamate; N-(3-{6-bromoimidazo[1,2-a]pyrimidin-2-yl}-4-fluorophenyl)formamide; tert-butyl N-[2-(2-fluoro-5-formamidophenyl)imidazo[1,2-a]pyrimidin-6-yl]carbamate; N-(4-fluoro-3-{6-formamidoimidazo[1,2-a]pyrimidin-2-yl}phenyl)pyrrolidine-1-carboxamide; N-{4-fluoro-3-[6-(5-oxo-4,5-dihydro-1H-1,2,4-triazol-4-yl)imidazo[1,2-a]pyrimidin-2-yl]phenyl}pyrrolidine-1-carboxamide; propan-2-yl N-{2-[2-chloro-5-(5-oxo-4,5-dihydro-1H-1,2,4-triazol-4-yl)phenyl]imidazo[1,2-a]pyrimidin-6-yl}carbamate; N-(4-fluoro-3-{6-methoxyimidazo[1,2-a]pyrimidin-2-yl}phenyl)pyrrolidine-1-carboxamide; N-{4-fluoro-3-[6-(prop-1-en-2-yl)imidazo[1,2-a]pyrimidin-2-yl]phenyl}-2,4-dimethyl-1,3-oxazole-5-carboxamide; N-{4-fluoro-3-[6-(piperidin-4-yl)imidazo[1,2-a]pyrimidin-2-yl]phenyl}-2,4-dimethyl-1,3-oxazole-5-carboxamide; methyl 2-(4-{2-[5-(dimethyl-1,3-oxazole-5-amido)-2-fluorophenyl]imidazo[1,2-a]pyrimidin-6-yl}piperidin-1-yl)acetate; N-(4-fluoro-3-{6-[1-(2-methoxyethyl)piperidin-4-yl]imidazo[1,2-a]pyrimidin-2-yl}phenyl)-2,4-dimethyl-1,3-oxazole-5-carboxamide; N-(4-fluoro-3-{6-[1-(2-hydroxyethyl)piperidin-4-yl]imidazo[1,2-a]pyrimidin-2-yl}phenyl)-2,4-dimethyl-1,3-oxazole-5-carboxamide; N-(4-fluoro-3-{6-[1-(2-hydroxyacetyl)piperidin-4-yl]imidazo[1,2-a]pyrimidin-2-yl}phenyl)-2,4-dimethyl-1,3-oxazole-5-carboxamide; N-{4-fluoro-3-[6-(4-hydroxybut-1-yn-1-yl)imidazo[1,2-a]pyrimidin-2-yl]phenyl}-2,4-dimethyl-1,3-oxazole-5-carboxamide; N-(4-fluoro-3-{6-[4-(morpholin-4-yl)but-1-yn-1-yl]imidazo[1,2-a]pyrimidin-2-yl}phenyl)-2,4-dimethyl-1,3-oxazole-5-carboxamide; N-(4-fluoro-3-{6-[4-(3-hydroxypiperidin-1-yl)but-1-yn-1-yl]imidazo[1,2-a]pyrimidin-2-yl}phenyl)-2,4-dimethyl-1,3-oxazole-5-carboxamide; N-(4-fluoro-3-{6-[4-(4-methylpiperazin-1-yl)but-1-yn-1-yl]imidazo[1,2-a]pyrimidin-2-yl}phenyl)-2,4-dimethyl-1,3-oxazole-5-carboxamide; N-{4-fluoro-3-[6-(2-methoxyethoxy)imidazo[1,2-a]pyrimidin-2-yl]phenyl}-2,4-dimethyl-1,3-oxazole-5-carboxamide; methyl 2-[5-(dimethyl-1,3-oxazole-5-amido)-2-fluorophenyl]imidazo[1,2-a]pyrimidine-6-carboxylate; N-{4-fluoro-3-[6-(trimethylsilyl)imidazo[1,2-a]pyrimidin-2-yl]phenyl}-2,4-dimethyl-1,3-oxazole-5-carboxamide; propan-2-yl N-{2-[5-(5-chlorofuran-2-amido)-2-fluorophenyl]imidazo[1,2-a]pyrimidin-6-yl}-N-methylcarbamate; propan-2-yl N-{2-[2-fluoro-5-(N-methyl5-chlorofuran-2-amido)phenyl]imidazo[1,2-a]pyrimidin-6-yl}-N-methylcarbamate; propan-2-yl N-{2-[5-(dimethyl-1,3-oxazole-5-amido)-2-fluorophenyl]imidazo[1,2-a]pyrimidin-6-yl}-N-methylcarbamate; propan-2-yl N-{2-[2-fluoro-5-(N-methyldimethyl-1,3-oxazole-5-amido)phenyl]imidazo[1,2-a]pyrimidin-6-yl}-N-methylcarbamate; propan-2-yl N-{2-[5-(dimethyl-1,3-oxazole-5-amido)-2-fluorophenyl]imidazo[1,2-a]pyrimidin-6-yl}-N-(methoxymethyl)carbamate; propan-2-yl N-{2-[5-(dimethyl-1,3-oxazole-5-amido)-2-fluorophenyl]imidazo[1,2-a]pyrimidin-6-yl}-N-(2-methoxyethyl)carbamate; ({2-[5-(dimethyl-1,3-oxazole-5-amido)-2-fluorophenyl]imidazo[1,2-a]pyrimidin-6-yl}[(propan-2-yloxy)carbonyl]amino)methyl butanoate; N-(4-fluoro-3-{6-[(propan-2-yl)amino]imidazo[1,2-a]pyrimidin-2-yl}phenyl)-N,2,4-trimethyl-1,3-oxazole-5-carboxamide; N-(3-{6-aminoimidazo[1,2-a]pyrimidin-2-yl}-4-fluorophenyl)-N-methylpyrrolidine-1-carboxamide; N-{3-[6-(azetidin-1-yl)imidazo[1,2-a]pyrimidin-2-yl]-4-fluorophenyl}-2,4-dimethyl-1,3-oxazole-5-carboxamide; propan-2-yl N-[2-(2-fluoro-5-{[2-oxo-2-(pyrrolidin-1-yl)ethyl]amino}phenyl)imidazo[1,2-a]pyrimidin-6-yl]carbamate; propan-2-yl N-{2-[2-chloro-5-(furan-2-amido)phenyl]imidazo[1,2-a]pyrimidin-6-yl}-N-methylcarbamate; N-{3-[6-(cyclopropylmethoxy)imidazo[1,2-a]pyrimidin-2-yl]-4-fluorophenyl}pyrrolidine-1-carboxamide; N-(4-fluoro-3-{6-[2-(morpholin-4-yl)ethoxy]imidazo[1,2-a]pyrimidin-2-yl}phenyl)pyrrolidine-1-carboxamide; N-(4-fluoro-3-{6-[(propan-2-yl)amino]imidazo[1,2-a]pyrimidin-2-yl}phenyl)azetidine-1-carboxamide; N-{4-fluoro-3-[6-(4-methylpiperazin-1-yl)imidazo[1,2-a]pyrimidin-2-yl]phenyl}-2,4-dimethyl-1,3-oxazole-5-carboxamide; N-{4-fluoro-3-[6-(trifluoroacetamido)imidazo[1,2-a]pyrimidin-2-yl]phenyl}pyrrolidine-1-carboxamide; N-[2-(5-acetamido-2-fluorophenyl)imidazo[1,2-a]pyrimidin-6-yl]acetamide; N-(5-{6-aminoimidazo[1,2-a]pyrimidin-2-yl}-2-fluorophenyl)pyrrolidine-1-carboxamide; N-(3-{6-aminoimidazo[1,2-a]pyrimidin-2-yl}-4-fluorophenyl)acetamide; N-{4-chloro-3-[6-(1,2,3,6-tetrahydropyridin-4-yl)imidazo[1,2-a]pyrimidin-2-yl]phenyl}pyrrolidine-1-carboxamide; propan-2-yl N-(2-{5-[(3-aminoazetidine-1-carbonyl)amino]-2-fluorophenyl}imidazo[1,2-a]pyrimidin-6-yl)carbamate; N-(4-fluoro-3-{6-[6-(piperazin-1-yl)pyridin-2-yl]imidazo[1,2-a]pyrimidin-2-yl}phenyl)-2,4-dimethyl-1,3-oxazole-5-carboxamide; N-{4-fluoro-3-[6-(1H-pyrrol-2-yl)imidazo[1,2-a]pyrimidin-2-yl]phenyl}-2,4-dimethyl-1,3-oxazole-5-carboxamide; N-(4-chloro-3-{6-[4-(piperidin-4-yloxy)phenyl]imidazo[1,2-a]pyrimidin-2-yl}phenyl)pyrrolidine-1-carboxamide; N-{4-fluoro-3-[6-(2,2,2-trifluoroethyl)imidazo[1,2-a]pyrimidin-2-yl]phenyl}-2,4-dimethyl-1,3-oxazole-5-carboxamide; 5-fluoro-N-(4-fluoro-3-{6-phenylimidazo[1,2-a]pyrimidin-2-yl}phenyl)furan-2-carboxamide; N-(3-{6-aminoimidazo[1,2-a]pyrimidin-2-yl}-4-chlorophenyl)-5-fluorofuran-2-carboxamide; N-[3-(6-{[1-(dimethylamino)propan-2-yl]amino}imidazo[1,2-a]pyrimidin-2-yl)-4-fluorophenyl]-2,4-dimethyl-1,3-oxazole-5-carboxamide; tert-butyl N-(2-{2-fluoro-3-[(pyrrolidine-1-carbonyl)amino]phenyl}imidazo[1,2-a]pyrimidin-6-yl)carbamate; N-(3-{6-aminoimidazo[1,2-a]pyrimidin-2-yl}-2-fluorophenyl)pyrrolidine-1-carboxamide; N-(3-{6-ethenylimidazo[1,2-a]pyrimidin-2-yl}-4-fluorophenyl)-2,4-dimethyl-1,3-oxazole-5-carboxamide; N-{3-[6-(ethylamino)imidazo[1,2-a]pyrimidin-2-yl]-4-fluorophenyl}-2,4-dimethyl-1,3-oxazole-5-carboxamide; 1-{3-[6-(dimethylamino)imidazo[1,2-a]pyrimidin-2-yl]-4-fluorophenyl}-3,3-dimethylurea; 4-fluoro-3-[6-(propan-2-yl)imidazo[1,2-a]pyrimidin-2-yl]aniline; N-(4-fluoro-3-{6-[5-(2-hydroxyethyl)pyridin-2-yl]imidazo[1,2-a]pyrimidin-2-yl}phenyl)-2,4-dimethyl-1,3-oxazole-5-carboxamide; 1-(3-{6-bromoimidazo[1,2-a]pyrimidin-2-yl}-4-fluorophenyl)-3-methyl-3-(2,2,2-trifluoroethyl)urea; 3-fluoro-N-{4-fluoro-3-[6-(trifluoromethyl)imidazo[1,2-a]pyrimidin-2-yl]phenyl}azetidine-1-carboxamide; N-(3-{6-[3-(difluoromethyl)pyridin-2-yl]imidazo[1,2-a]pyrimidin-2-yl}-4-fluorophenyl)-2,4-dimethyl-1,3-oxazole-5-carboxamide; N-(4-fluoro-3-{6-[3-(trifluoromethyl)pyridin-2-yl]imidazo[1,2-a]pyrimidin-2-yl}phenyl)-2,4-dimethyl-1,3-oxazole-5-carboxamide; N-{4-fluoro-3-[6-(2-methylpropyl)imidazo[1,2-a]pyrimidin-2-yl]phenyl}-2,4-dimethyl-1,3-oxazole-5-carboxamide; N-{3-[6-(2,2-dimethylpropyl)imidazo[1,2-a]pyrimidin-2-yl]-4-fluorophenyl}-2,4-dimethyl-1,3-oxazole-5-carboxamide; 3-fluoro-N-(4-fluoro-3-{6-[(propan-2-yl)amino]imidazo[1,2-a]pyrimidin-2-yl}phenyl)azetidine-1-carboxamide; N-{3-[6-(dimethylamino)imidazo[1,2-a]pyrimidin-2-yl]-4-fluorophenyl}-3-fluoroazetidine-1-carboxamide; 4-fluoro-3-[6-(3-methylpyridin-2-yl)imidazo[1,2-a]pyrimidin-2-yl]aniline; N-(3-{6-cyclopropylimidazo[1,2-a]pyrimidin-2-yl}-4-fluorophenyl)-5-(3-methyloxetan-3-yl)-1,2,4-oxadiazol-3-amine; 2-{3-[(3-{6-cyclopropylimidazo[1,2-a]pyrimidin-2-yl}-4-fluorophenyl)amino]-1,2,4-oxadiazol-5-yl}-2-methylpropan-1-ol; N-(3-{6-cyclopropylimidazo[1,2-a]pyrimidin-2-yl}-4-fluorophenyl)-4-methoxycyclohexane-1-carboxamide; (1 s,4s)-N-(3-{6-cyclopropylimidazo[1,2-a]pyrimidin-2-yl}-4-fluorophenyl)-4-hydroxycyclohexane-1-carboxamide; N-(3-{6-cyclopropylimidazo[1,2-a]pyrimidin-2-yl}-4-fluorophenyl)-3-oxocyclopentane-1-carboxamide; N-(3-{6-cyclopropylimidazo[1,2-a]pyrimidin-2-yl}-4-fluorophenyl)-1,4-dioxane-2-carboxamide; N-(3-{6-cyclopropylimidazo[1,2-a]pyrimidin-2-yl}-4-fluorophenyl)-4-methylmorpholine-2-carboxamide; tert-butyl 2-[(3-{6-cyclopropylimidazo[1,2-a]pyrimidin-2-yl}-4-fluorophenyl)carbamoyl]morpholine-4-carboxylate; tert-butyl 3-[(3-{6-cyclopropylimidazo[1,2-a]pyrimidin-2-yl}-4-fluorophenyl)carbamoyl]morpholine-4-carboxylate; N-(3-{6-cyclopropylimidazo[1,2-a]pyrimidin-2-yl}-4-fluorophenyl)-3,3-difluoroazetidine-1-carboxamide; (3R)—N-(3-{6-cyclopropylimidazo[1,2-a]pyrimidin-2-yl}-4-fluorophenyl)-3-fluoropyrrolidine-1-carboxamide; 1-(3-{6-cyclopropylimidazo[1,2-a]pyrimidin-2-yl}-4-fluorophenyl)-3,3-bis(²H₃)methylurea; N-(4-fluoro-3-{6-methoxyimidazo[1,2-a]pyrimidin-2-yl}phenyl)-2,4-dimethyl-1,3-oxazole-5-carboxamide; N-{4-fluoro-3-[6-(propan-2-yloxy)imidazo[1,2-a]pyrimidin-2-yl]phenyl}-2,4-dimethyl-1,3-oxazole-5-carboxamide; methyl 2-({2-[5-(dimethyl-1,3-oxazole-5-amido)-2-fluorophenyl]imidazo[1,2-a]pyrimidin-6-yl}oxy)acetate; N-{3-[6-(cyclopropylmethoxy)imidazo[1,2-a]pyrimidin-2-yl]-4-fluorophenyl}-2,4-dimethyl-1,3-oxazole-5-carboxamide; N-{4-fluoro-3-[6-(pyridin-2-ylmethoxy)imidazo[1,2-a]pyrimidin-2-yl]phenyl}-2,4-dimethyl-1,3-oxazole-5-carboxamide; N-{4-fluoro-3-[6-(propan-2-yloxy)imidazo[1,2-a]pyrimidin-2-yl]phenyl}-2,4-dimethyl-N-(propan-2-yl)-1,3-oxazole-5-carboxamide; N-{3-[6-(cyclobutylmethyl)imidazo[1,2-a]pyrimidin-2-yl]-4-fluorophenyl}-2,4-dimethyl-1,3-oxazole-5-carboxamide; N-{3-[6-(1,3-dioxolan-2-ylmethyl)imidazo[1,2-a]pyrimidin-2-yl]-4-fluorophenyl}-2,4-dimethyl-1,3-oxazole-5-carboxamide; N-[3-(6-{bicyclo[3.1.1]heptan-6-yl}imidazo[1,2-a]pyrimidin-2-yl)-4-fluorophenyl]-2,4-dimethyl-1,3-oxazole-5-carboxamide; N-{4-fluoro-3-[6-(morpholin-4-ylmethyl)imidazo[1,2-a]pyrimidin-2-yl]phenyl}-2,4-dimethyl-1,3-oxazole-5-carboxamide; N-(3-{6-[(dimethylamino)methyl]imidazo[1,2-a]pyrimidin-2-yl}-4-fluorophenyl)-2,4-dimethyl-1,3-oxazole-5-carboxamide; N-(4-fluoro-3-{6-[1-(morpholin-4-yl)ethyl]imidazo[1,2-a]pyrimidin-2-yl}phenyl)-2,4-dimethyl-1,3-oxazole-5-carboxamide; and N-(3-{6-[1-(dimethylamino)ethyl]imidazo[1,2-a]pyrimidin-2-yl}-4-fluorophenyl)-2,4-dimethyl-1,3-oxazole-5-carboxamide.

It is noted that the compounds of the present invention may be in the form of a pharmaceutically acceptable salt. It is further note that the compounds of the present invention may be a mixture of stereoisomers, or the compound may comprise a single stereoisomer.

Further compounds of the invention are detailed in the Examples, infra.

In another aspect, the present invention is directed to a pharmaceutical composition which includes as an active ingredient a compound according to any one of the above embodiments and variations in combination with a pharmaceutically acceptable carrier, diluent or excipient.

In another embodiment, the pharmaceutical composition is a solid formulation adapted for oral administration. In another embodiment, the composition is a liquid formulation adapted for oral administration. In yet another embodiment, the composition is a tablet. In still another embodiment, the composition is a liquid formulation adapted for parenteral administration.

In yet another embodiment, the pharmaceutical composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In another aspect, the present application is directed to a compound or a pharmaceutical composition according to any one of the above embodiments and variations for use in a therapeutic application.

In another aspect, the present application is directed to a compound or a pharmaceutical composition according to any one of the above embodiments and variations for use as a medicament.

In still another aspect, the present application is directed to a compound or a pharmaceutical composition according to any one of the above embodiments and variations for treating, preventing, inhibiting, ameliorating, or eradicating the pathology and/or symptomology of a parasitic disease, wherein parasitic disease is Leishmaniasis, Human African Trypanosomiasis, or Chagas disease. Further the treatment may further include a second agent which may be other drugs that are known for treating Leishmaniasis, Human African Trypanosomiasis, or Chagas diseases. In one particular variation of treating Leishmaniasis, the second agent is selected from meglumine antimoniate, stibogluconate, Amphotericin, Miltefosine and paromomycin. In another particular variation of treating Human African Trypanosomiasis, the second agent is selected from pentamidine, suramin, melarsoprol, and eflornithine. In yet another particular variation of treating Chagas disease, the second agent is selected from benznidazole, nifurtimox or Amphotericin b.

In yet another aspect, the present invention is directed to a method for treating, preventing, inhibiting, ameliorating, or eradicating the pathology and/or symptomology of a parasitic disease. The method involves administering to a subJect a therapeutically effective amount of a compound or a pharmaceutical composition according to the above embodiments and variations.

In one embodiment of the method of the invention, the disease being treated is Leishmaniasis, Human African Trypanosomiasis, or Chagas disease.

In one embodiment of the method of the invention, the disease being treated is Leishmaniasis caused by the parasite Leishmania donovani, Leishmania infantum, Leishmania braziliensis, Leishmania panamensis, Leishmania guayanensis, Leishmania amazonensis, Leishmania mexicana, Leishmania tropica, or Leishmania major.

In a embodiment, the disease being treated is visceral Leishmaniasis caused by the parasite Leishmania donovani.

In another embodiment, the disease being treated is Human African Trypanosomiasis caused by Trypanosoma brucei, particularly, by the sub-species T.b. gambiense or T.b. rhodesiense.

In still another embodiment of the method of the invention, the disease being treated is Chagas disease, (also call American Trypanosomiasis) caused by Trypanosoma cruzi.

In the above method of the invention, the compounds or pharmaceutical compositions may be administered prior to, simultaneously with, or after a second agent. The second agent can be other drugs that are known for treating Leishmaniasis, Human African Trypanosomiasis, or Chagas diseases. In one particular variation for treating Leishmaniasis, the second agent is selected from meglumine antimoniate, stibogluconate, Amphotericin, Miltefosine and paromomycin. In another variation, for treating Human African Trypanosomiasis, the second agent is selected from pentamidine, suramin, melarsoprol, and eflornithine. In another particular variation of the method, for treating Chagas disease, the second agent is selected from benznidazole, nifurtimox or Amphotericin b.

In another aspect, the invention is directed to a compound, salt, steroisomer, or pharmaceutical composition thereof, according to any one of the above embodiments or variation, for treating, preventing, inhibiting, ameliorating, or eradicating the pathology and/or symptomology of a disease caused by the parasite Leishmania donovani, Leishmania infantum, Leishmania braziliensis, Leishmania panamensis, Leishmania guayanensis, Leishmania amazonensis, Leishmania mexicana, Leishmania tropica, Leishmania maJor, Trypanosoma cruzi, or Trypanosoma brucei. In one embodiment, the disease is visceral Leishmaniasis caused by Leishmania donovani. In another embodiment, the disease is Human African Trypanosomiasis caused by Trypanosoma brucei. In yet another embodiment, the disease is Chagas disease caused by Trypanosoma cruzi.

In still another aspect, the present invention is directed to the use of the compound, or a salt, a stereoisomer, or a pharmaceutical composition thereof, according to the any one of the above embodiments or variations in the manufacture of a medicament for treating, preventing, inhibiting, ameliorating, or eradicating the pathology and/or symptomology of a disease caused by Leishmania donovani, Leishmania infantum, Leishmania braziliensis, Leishmania panamensis, Leishmania guayanensis, Leishmania amazonensis, Leishmania mexicana, Leishmania tropica, Leishmania maJor, Trypanosoma cruzi, or Trypanosoma brucei. In one embodiment, the medicament is for treating visceral Leishmaniasis caused by Leishmania donovani. In another embodiment, the medicament is for treating Human African Trypanosomiasis caused by Trypanosoma brucei. In yet another embodiment, the medicament is for treating Chagas disease caused by Trypanosoma cruzi.

The medicament, in addition to the compound of the invention, may further include a second agent. The second agent may be other drugs that are known for treating Leishmaniasis, Human African Trypanosomiasis, or Chagas diseases. In one particular variation of the medicament, for treating Leishmaniasis, the second agent is selected from meglumine antimoniate, stibogluconate, Amphotericin, Miltefosine and paromomycin. In another particular variation of the medicament, for treating Human African Trypanosomiasis, the second agent is selected from pentamidine, suramin, melarsoprol, and eflornithine. In yet another particular variation of the medicament, for treating Chagas disease, the second agent is selected from benznidazole, nifurtimox or Amphotericin b.

In another aspect, the invention is related to a kit which comprises a compound of any one of the above embodiments and variations, and optionally a second therapeutic agent. In one particular variation, the kit comprises the compound in a multiple dose form.

### Enumerated Embodiments

Various enumerated embodiments of the invention are described herein. It will be recognized that features specified in each embodiment may be combined with other specified features to provide further embodiments of the present invention.

In a first embodiment, the invention provides a compound of Formula A:

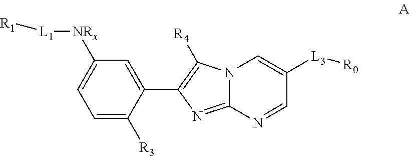

or a pharmaceutically acceptable salt, or stereoisomer thereof; wherein

Rₓ is hydrogen or C₁₋₄alkyl;

L₁ is a bond, —CH₂C(O)—, —C(O)— or —S(O)₂—;

R₁ is selected from hydrogen, nitro, C₁₋₄alkyl, C₂₋₄alkenyl, C₁₋₆alkoxy, —NR₇ₐR_{7b}, C₃₋₆cycloalkyl, C₄₋₇heterocycloalkyl, C₅₋₆heterocycloalkenyl, phenyl, benzyl and C₅₋₉heteroaryl; or —NRₓL₁R₁ is nitro; or R₁ and Rₓ with L₁ and N to which R₁ and Rₓ are respectivelyt attached are taken together to form a C₄₋₉heterocycylyl optionally substituted by 1 or 2 oxo, wherein
R₇ₐ is hydrogen or C₁₋₄alkyl;
R_{7b} is selected from hydrogen, C₁₋₄alkyl, C₁₋₄haloalkyl, C₁₋₄alkoxy, amino, C₃₋₆cycloalkyl, C₁₋₄alkoxy substituted C₃₋₆cycloalkyl, and C₅₋₆heteroaryl, wherein the C₅₋₆heteroaryl is optionally substituted by 1 to 2 substituents independently selected from halo, C₁₋₄alky, C₁₋₄alkoxyl and amino; and wherein the available hydrogens on the C₁₋₄alkyl of R₇ₐ and R_{7b} are each optionally replaced by deuterium;
the C₁₋₄alkyl, C₁₋₄alkenyl, C₁₋₆alkoxy, amino, C₃₋₆cycloalkyl, C₄₋₇heterocycloalkyl, C₅₋₆heterocycloalkenyl, phenyl, or C₅₋₉heteroaryl of R₁ is optionally substituted by 1 to 4 substituents independently selected from hydroxy, halo, cyano, oxo, C₁₋₄alkyl, C₁₋₄hydroxyalkyl, C₁₋₄haloalkyl, C₁₋₆alkoxy, C₁₋₄haloalkoxy, C₁₋₄alkylcarbonyloxy, hydroxycarbonyl, C₁₋₄alkylcarbonyl, C₁₋₄alkoxycarbonyl, aminocarbonyl, C₁₋₄alkylsulfonyl, amino, C₁₋₄alkylamino, C₁₋₄alkylcarbonylamino, C₁₋₄alkylcarbonyl-N—C₁₋₄alkylamino, C₁₋₆alkoxycarbonylamino, C₁₋₄alkoxycarbonyl-N—C₁₋₄alkylamino, C₁₋₆alkoxy substituted C₃₋₆cycloalkyl, C₃₋₆cycloalkyl, C₄₋₆heterocycloalkyl, phenyl, and C₅₋₆heteroaryl; wherein the C₃₋₆cycloalkyl, C₄₋₆heterocycloalkyl, C₅₋₆heterocycloalkyl, phenyl or C₅₋₆heteroaryl substituent is optionally substituted by 1 to 2 substituent independently selected from halo, C₁₋₄alkyl, C₁₋₄alkylamino, or C₄₋₆heterocycloalkyl;

R₃ is selected from hydrogen, halo, cyano, C₁₋₄alkyl and C₁₋₄haloalkyl;

R₄ is selected from hydrogen, halo, C₁₋₄alkyl, C₁₋₄haloalkyl, and —C(O)R₁₀, wherein R₁₀ is selected from hydroxy, C₁₋₄alkyl, C₁₋₄alkoxy, amino, C₁₋₄alkylamino, C₃₋₆cycloalkyl and C₄₋₆heterocycloalkyl, wherein the C₁₋₄alkyl, C₁₋₄alkoxy, amino, C₁₋₄alkylamino, C₃₋₆cycloalkyl or C₄₋₆heterocycloalkyl of R₁₀ is optionally substituted by 1 to 2 substituents independently selected from hydroxyl, halo and C₁₋₄alkyl;

L₃ is selected from a bond, C₃₋₇cycloalkyl, C₄₋₇heterocycloalkyl, C₄₋₇cycloalkenyl, C₆₋₇heterocycloalkenyl, phenyl, and C₅₋₆heteroaryl;

R₀ is selected from hydrogen, hydroxy, halo, oxo, nitro, —N═CHN(CH₃)₂, C₁₋₆alkyl, C₁₋₄haloalkyl, C₂₋₄alkenyl, C₂₋₄alkynyl, C₁₋₄alkoxy, C₄₋₆heterocycloalkyloxy, —C(O) R₆, —NR₂ₐR_{2b}, —NR₅C(O)R₆, —NR₆S(O)₂R₈, —S(O)₂R₈, tri-C₁₋₄alkylsilyl, C₃₋₆cycloalkyl, C₄₋₆heterocycloalkyl, C₃₋₆cycloalkenyl, C₄₋₆heterocycloalkenyl, phenyl, and C₅₋₆heteroaryl; provided when L₃ is a bond, R₀ is not hydrogen; wherein
the C₁₋₆alkyl, C₂₋₄alkenyl, C₂₋₄alkynyl, or C₁₋₄alkoxy of R₀ is optionally substituted by 1-2-substituents independently selected from hydroxy, halo, C₁₋₄alkoxy, trimethylsilylC₁₋₄alkoxy, C₁₋₄alkoxycarbonyl, —NR₁₁ₐR_{11b}, C₁₋₄alkoxycarbonylamino, C₅₋₆heterocycloalkyloxy, C₃₋₇cycloalkyl, C₅₋₆heterocycloalkyl, phenyl and C₅₋₆heteroaryl, wherein the C₃₋₇cycloalkyl, C₅₋₆heterocycloalkyl, phenyl or C₅₋₆heteroaryl substituent is optionally substituted by hydroxy, halo, C₁₋₄alkyl or C₁₋₄alkoxycarbonylamino, and wherein R₁₁ₐ and R_{11b} are independently hydrogen or C₁₋₄alkyl;
the C₄₋₆heterocycloalkyloxy, C₃₋₆cycloalkyl, C₄₋₆heterocycloalkyl, C₄₋₆heterocycloalkenyl, phenyl or C₅₋₆heteroaryl of R₀ is optionally substituted by 1 to 4 substituents independently selected from hydroxy, halo, oxo, C₁₋₄alkyl, C₁₋₄hydroxyalkyl, C₁₋₄haloalkyl, —(CH₂)₀₋₄NRₐR_{b}, C₅₋₆arylC₁₋₄alkyl, C₄₋₆heterocycloalkylC₁₋₄alkyl, C₁₋₄alkoxy, C₁₋₄alkoxycarbonyl, C₁₋₄alkylsulfonyl, C₄₋₆heterocycloalkyl, wherein Rₐ and R_{b} are each independently hydrogen, C₁₋₄alkyl or C₃₋₆cycloalkyl;

R₂ₐ is selected from hydrogen, C₁₋₄alkyl, C₁₋₄alkoxyC₁₋₄alkyl, C₁₋₄alkylcarbonyloxyC₁₋₄alkyl, and a C₁₋₄haloalkyl substituted C₅₋₆heteroaryl;
R_{2b} is selected from hydrogen, C₁₋₆alkyl, C₁₋₆haloalkyl, aminoC₁₋₄alkyl, C₁₋₄alkoxy, C₁₋₄alkoxycarbonyl, C₃₋₆cycloalkyl, C₄₋₆heterocycloalkyl, C₃₋₆heterocycloalkenyl, and C₅₋₆heteroaryl, wherein
   the C₁₋₆alkyl of R_{2b} is optionally substituted by C₁₋₄alkoxy, amino, C₁₋₄alkylamino, C₄₋₆cycloalkyl, C₄₋₆heterocycloalkyl, phenyl or C₅₋₆heteroaryl, wherein the C₄₋₆cycloalkyl, C₄₋₆heterocycloalkyl, phenyl or C₅₋₆heteroaryl substituent is optionally substituted by hydroxy, halo, C₁₋₄alkyl, C₁₋₄haloalkyl, or C₁₋₄alkyoxycarbonylamino;
   the C₄₋₆heterocycloalkyl, C₅₋₆heterocycloalkenyl or C₅₋₆heteroaryl of R_{2b} is optionally substituted by 1 to 2 substituents independently selected from C₁₋₄alkyl and C₁₋₄haloalkyl;
R₅ is hydrogen or C₁₋₄alkyl;
R₆ is selected from hydrogen, C₁₋₆alkyl, C₁₋₆hydroxyalkyl, C₁₋₆haloalkyl, C₂₋₆alkenyl, C₁₋₄alkoxy, C₁₋₆haloalkoxy, C₁₋₄cycloalkoxy, C₂₋₆alkenyloxy, C₃₋₆cycloalkyloxy, benzyloxy, —NR₁₂ₐR_{12b}, C₃₋₇cycloalkyl, C₄₋₇heterocycloalkyl, C₅₋₇cycloalkenyl, C₅₋₆heterocycloalkenyl, phenyl, and C₅₋₆heteroaryl, wherein
   R₁₂ₐ is hydrogen or C₁₋₆alkyl,
   R_{12b} is selected from hydrogen, C₁₋₆alkyl, amino and C₁₋₆alkylamino;
   the C₁₋₆alkyl, C₂₋₆alkenyl, C₁₋₄alkoxy, C₃₋₆cycloalkyloxy, or amino of R₆ is optionally substituted by 1 to 2 substituents independently selected from hydroxy, halo, C₁₋₄alkyl, C₁₋₄haloalkyl, C₁₋₄alkoxy, C₁₋₄haloalkoxy, C₁₋₄alkoxyC₁₋₄alkoxy, —NR₉ₐR_{9b}, C₃₋₆cycloalkyl, C₄₋₇heterocycloalkyl, and C₅₋₆heteroaryl, wherein the C₅₋₆heterocycloalkyl or C₅₋₆heteroaryl substituent is optionally substituted by 1-2 substituents independently selected from hydroxyl, C₁₋₄alkyl and C₁₋₄alkoxycarbonyl, and wherein R₉ₐ is hydrogen or C₁₋₄alkyl and R_{9b} is selected from hydrogen, C₁₋₄alkyl, C₁₋₄alkylcarbonyl and C₁₋₄alkoxycarbonyl;
   the C₃₋₆cycloalkyloxy, C₃₋₇cycloalkyl, C₄₋₇heterocycloalkyl, C₅₋₇cycloalkenyl, or C₅₋₆heterocycloalkenyl of R₆ is optionally substituted by 1 to 3 substituents independently selected from hydroxy, halo, cyano, oxo, C₁₋₄alkyl, haloC₁₋₄alkyl, C₁₋₄alkoxylC₁₋₄alkyl, C₁₋₄alkoxycarbonylaminoC₁₋₄alkyl, C₁₋₄alkoxyl, C₁₋₄alkoxycarbonyl, amino, aminocarbonyl, and the phenyl or C₅₋₆heteroaryl of R₆ is optionally substituted with 1-2 substituents independently selected from hydroxy, halo, cyano, C₁₋₄alkyl, C₁₋₄hydroxyalkyl, C₁₋₄haloalkyl, C₁₋₄alkoxy, C₁₋₄alkoxycarbonyl, —(CH₂)₀₋₄NR₁₃ₐR_{13b}, and C₁₋₄alkoxycarbonylamino, wherein the C₁₋₄alkoxy substituent is optionally substituted by C₁₋₄alkylC₅₋₆heterocycloalkyl, and wherein R₁₃ₐ and R_{13b} are each independently hydrogen or C₁₋₄alkyl; and

R₈ is C₁₋₄alkyl or C₁₋₄alkylamino.

### Embodiment 2

The compound of Formula A, or a pharmaceutically acceptable salt, or stereoisomer thereof; according to Embodiment 1,

Rₓ is hydrogen;

L₁ is a bond or —C(O)—;

R₁ is selected from C₁₋₆alkoxy, C₁₋₆alkoxyC₁₋₆alkoxy, NR₇ₐR_{7b}, C₃₋₆cycloalkyl, C₄₋₇heterocycloalkyl, C₅₋₆heterocycloalkenyl, phenyl, benzyl, and C₅₋₉heteroaryl, wherein
R₇ₐ is hydrogen or C₁₋₄alkyl;
R_{7b} is selected from hydrogen, C₁₋₄alkyl, C₁₋₄haloalkyl, C₁₋₄alkoxy, amino, C₁₋₄alkoxy substituted C₃₋₆cycloalkyl, C₃₋₆cycloalkyl, and C₅₋₆heteroaryl, wherein the C₅₋₆heteroaryl is optionally substituted by halo or C₁₋₄alkyl;
the C₃₋₆cycloalkyl, C₄₋₇heterocycloalkyl, C₅₋₆heterocycloalkenyl, phenyl, or C₅₋₉heteroary of R₁ is optionally substituted by 1-2 substituents independently selected from hydroxy, halo, cyano, oxo, C₁₋₄alkyl, C₁₋₄haloalkyl, C₁₋₄alkoxy, amino, C₁₋₄alkylamino, C₁₋₄alkylcarbonyl, aminocarbonyl, C₁₋₄alkylsulfonyl, C₃₋₆cycloalkyl, and C₄₋₆heterocycloalkyl; R₃ is hydrogen or halo;

R₄ is hydrogen or halo;

L₃ is a selected from a bond, C₃₋₇cycloalkyl, C₄₋₇heterocycloalkyl, C₅₋₆cycloalkenyl,

C₅₋₆heterocycloalkenyl, phenyl, and C₅₋₆heteroaryl;

R₀ is selected from hydrogen, halo, oxo, C₁₋₆alkyl, C₁₋₄haloalkyl, C₂₋₄alkenyl, C₂₋₄alkynyl, C₁₋₄alkoxy, C₄₋₆heterocycloalkyloxy, —C(O) R₆, —NR₂ₐR_{2b}, —NR₅C(O)R₆, -tri-C₁₋₄alkylsilyl, C₄₋₆heterocycloalkyl, C₄₋₆heterocycloalkenyl, phenyl, and C₅₋₆heteroaryl; provided when L₃ is a bond, R₀ is not hydrogen; wherein
the C₁₋₆alkyl, C₂₋₄alkynyl or C₁₋₄alkoxy of R₀ is optionally substituted by 1 to 2 substituents independently selected from hydroxy, C₁₋₄alkoxy, trimethylsilylC₁₋₄alkoxy, C₁₋₄alkoxycarbonyl, —NR₁₁ₐR_{11b}, C₁₋₄alkoxycarbonylamino, C₃₋₇cycloalkyl, C₅₋₆heterocycloalkyl, phenyl and C₅₋₆heteroaryl; wherein the C₃₋₇cycloalkyl or C₅₋₆heterocycloalkyl substituent is optionally substituted by hydroxy, C₁₋₄alkyl or C₁₋₄alkoxycarbonylamino, and wherein R₁₁ₐ and R_{11b} are independently hydrogen or C₁₋₄alkyl;
the C₄₋₆heterocycloalkyloxy, C₄₋₆heterocycloalkyl, C₄₋₆heterocycloalkenyl, phenyl or C₅₋₆heteroaryl of R₀ is optionally substituted by 1 to 4 substituents independently selected from hydroxy, halo, C₁₋₄alkyl, C₁₋₄haloalkyl, —(CH₂)₀₋₄NRₐR_{b}, C₅₋₆arylC₁₋₄alkyl, C₄₋₆heterocycloalkylC₁₋₄alkyl, C₁₋₄alkoxy, C₁₋₄alkoxycarbonyl, C₁₋₄alkylsulfonyl, and C₄₋₆heterocycloalkyl, wherein Rₐ and R_{b} are independently hydrogen or C₁₋₄alkyl;
R₂ₐ is selected from hydrogen, C₁₋₄alkyl, C₁₋₄alkoxyC₁₋₄alkyl, C₁₋₄alkylcarbonyloxyC₁₋₄alkyl, and a C₁₋₄haloalkyl substituted C₅₋₆heteroaryl;
R_{2b} is selected from hydrogen, C₁₋₄alkyl, C₁₋₄haloalkyl, aminoC₁₋₄alkyl, C₁₋₄alkoxy, C₁₋₄alkoxycarbonyl, C₃₋₆cycloalkyl, C₄₋₆heterocycloalkyl, C₅₋₆heterocycloalkenyl, and C₅₋₆heteroaryl, wherein
   the C₁₋₄alkyl of R_{2b} is optionally substituted by C₁₋₄alkoxy, C₁₋₄alkylamino, C₄₋₆cycloalkyl, or C₅₋₆heteroaryl, wherein the C₄₋₆cycloalkyl substituent is optionally substituted by C₁₋₄alkyoxycarbonylamino; and
   the C₄₋₆heterocycloalkyl, C₅₋₆heterocycloalkenyl or C₅₋₆heteroaryl of
R_{2b} is optionally substituted by 1 to 2 substituents independently selected from C₁₋₄alkyl and C₁₋₄haloalkyl;
R₅ is hydrogen or C₁₋₄alkyl;
R₆ is selected from hydrogen, C₁₋₆alkyl, C₁₋₆hydroxyalkyl, C₁₋₄haloalkyl, C₁₋₄alkoxy, C₂₋₄alkenyloxy, C₃₋₆cycloalkyloxy, benzyloxy, —NR₁₂ₐR_{12b}, C₃₋₇cycloalkyl, C₄₋₇heterocycloalkyl, C₅₋₇cycloalkenyl, C₅₋₆heterocycloalkenyl, phenyl, and C₅₋₆heteroaryl, wherein
   R₁₂ₐ is hydrogen or C₁₋₆alkyl;
   R_{12b} is selected from hydrogen, C₁₋₆alkyl, amino and C₁₋₆alkylamino;
   the C₁₋₆alkyl or C₁₋₄alkoxy of R₆ is optionally substituted by 1 to 2 substituents independently selected from halo, hydroxy, C₁₋₄haloalkyl, C₁₋₄alkoxy, C₁₋₄haloalkoxy, C₁₋₄alkoxyC₁₋₄alkoxy, —NR₉ₐR_{9b}, and C₄₋₇heterocycloalkyl, wherein R₉ₐ is hydrogen or C₁₋₄alkyl and R_{9b} is selected from hydrogen, C₁₋₄alkyl, and C₁₋₄alkoxycarbonyl;
   the C₃₋₆cycloalkyloxy, C₃₋₇cycloalkyl, C₄₋₇heterocycloalkyl, C₅₋₇cycloalkenyl, or C₅₋₆heterocycloalkenyl of R₆ is optionally substituted by 1 to 3 substituents independently selected from hydroxy, halo, cyano, oxo, C₁₋₆alkyl, C₁₋₄alkoxylC₁₋₄alkyl, C₁₋₄alkoxy, C₁₋₄alkoxycarbonyl, and amino;
   the phenyl or C₅₋₆heteroaryl of R₆ is optionally substituted with 1 to 2 substituents independently selected from halo, cyano, C₁₋₄alkyl, C₁₋₄hydroxyalkyl, C₁₋₄haloalkyl, C₁₋₄alkoxy, C₁₋₄alkoxycarbonyl, —(CH₂)₀₋₄NR₁₃ₐR_{13b}, and C₁₋₄alkoxycarbonylamino, wherein the C₁₋₄alkoxy substituent is optionally substituted by C₁₋₄alkylC₅₋₆heterocycloalkyl, and wherein R₁₃ₐ and R_{13b} are independently hydrogen or C₁₋₄alkyl; and R₈ is C₁₋₄alkyl.

### Embodiment 3

A compound of Formula A, or a pharmaceutically acceptable salt, or stereoisomer thereof; according to any one of Embodiments 1 to 2, wherein R₁ is selected from C₁₋₆alkoxy, C₃₋₆cycloalkyl, C₄₋₇heterocycloalkyl, C₅₋₆heterocycloalkenyl, phenyl, benzyl, and C₅₋₉heteroaryl, wherein the C₃₋₆cycloalkyl, C₄₋₇heterocycloalkyl, C₅₋₆heterocycloalkenyl, phenyl, or C₅₋₉ heteroaryl is optional substituted by 1 to 2 substituents independently selected from hydroxy, halo, cyano, oxo, C₁₋₄alkyl, C₁₋₄haloalkyl, C₁₋₄alkoxy, amino, C₁₋₄alkylamino, C₁₋₄alkylcarbonyl, aminocarbonyl, C₁₋₄alkylsulfonyl, C₃₋₆cycloalkyl, and C₄₋₆heterocycloalkyl.

### Embodiment 4

A compound of Formula A, or a pharmaceutically acceptable salt, or stereoisomer thereof; according to any one of Embodiments 1 to 2, wherein R₁ is selected methoxy, ethoxy, isopropyloxy, cyclopropyl, cyclobutyl, cyclopentyl, cyclohexyl, azetidinyl, pyrrolidinyl, piperidinyl, piperazinyl, morpholinyl, tetrahydropyranyl, dioxanyl, 5-azaspiro[2,4]heptanyl, 2-oxa-6-aza-spiro[3,3]heptanyl, oxaazobicyclo[2.2.1]heptanyl, dihydrooxazolyl, phenyl, benzyl, pyrrolyl, pyrazolyl, imidazolyl, furanyl, isooxazolyl, oxazolyl, thiazolyl, pyridinyl, pyrimidinyl, indolyl, furo[2,3-c]pyridinyl, and imidazo[1,2-a]pyrimidinyl, wherein the above C₃₋₆cycloalkyl, C₄₋₇heterocycloalkyl, C₅₋₆heterocycloalkenyl, phenyl, or C₅₋₉heteroaryl is optional substituted by 1 to 2 substituents independently selected from hydroxy, halo, cyano, oxo, methyl, trifluoromethyl, methoxy, amino, —NHCH₃, methylcarbonyl, aminocarbonyl, methylsulfonyl, cyclopropyl, and morpholinyl.

### Embodiment 5

A compound of Formula A, or a pharmaceutically acceptable salt, or stereoisomer thereof; according to any one of Embodiments 1 to 4, wherein R₀ is selected from hydrogen, halo, oxo, C₁₋₆alkyl, C₁₋₄hydroxyalkyl, C₁₋₄haloalkyl, C₂₋₄alkenyl, trimethylsilylC₁₋₆alkyloxyC₁₋₆alkyl, —NR₂ₐR_{2b}, —NHC(O)R₆, C₄₋₆heterocycloalkyl, C₄₋₆heterocycloalkenyl, phenyl and C₅₋₆heteroaryl, wherein

the C₄₋₆heterocycloalkyl, C₄₋₆heterocycloalkenyl, phenyl or C₅₋₆heteroaryl is optionally substituted by 1 to 3 substituents independently selected from hydroxy, halo and C₁₋₄alkyl, C₁₋₄haloalkyl, and C₁₋₄alkoxycarbonyl;

R₂ₐ is selected from hydrogen, C₁₋₄alkyl, C₁₋₆alkoxyC₁₋₄alkyl, C₁₋₄alkylcarbonyloxyC₁₋₄alkyl, and a C₁₋₄haloalkyl substituted C₅₋₆heteroaryl;

R_{2b} is hydrogen, C₁₋₄alkyl, C₁₋₄alkoxy, or C₁₋₄alkoxycarbonyl;

R₆ is selected from C₁₋₄alkoxy, C₃₋₇cycloalkyl, C₄₋₇heterocycloalkyl, C₅₋₇cycloalkenyl, C₅₋₆heterocycloalkenyl, phenyl and C₅₋₆heteroaryl, wherein the C₃₋₇cycloalkyl, C₄₋₇heterocycloalkyl, C₅₋₇cycloalkenyl, or C₅₋₆heterocycloalkenyl is optionally substituted by 1 to 2 substituents independently selected from hydroxy, halo, cyano, oxo, C₁₋₆alkyl, C₁₋₄alkoxylC₁₋₄alkyl, C₁₋₄alkoxy, C₁₋₄alkoxycarbonyl, and amino, and wherein the phenyl or C₅₋₆heteroaryl is optionally substituted by 1 to 2 substituents independently selected from halo, cyano, C₁₋₆alkyl, C₁₋₄hydroxyalkyl, C₁₋₄haloalkyl, C₁₋₄alkoxycarbonyl, amino, C₁₋₄alkyamino, and C₁₋₄alkoxycarbonylamino.

### Embodiment 6

A compound of Formula A, or a pharmaceutically acceptable salt, or stereoisomer thereof; according to any one of Embodiments 1 to 4, wherein R₀ is selected from hydrogen, halo, oxo, methyl, ethyl, isopropyl, isobutyl, isohexyl, —CH═CH₂, —(CH₂)₂OH, —CHF₂, —CF₃, —CH₂CF₃, —(CH₂)₂CF₃, trimethylsilylethoxymethyl, —NHCH₃, —NH— trifluoromethylpyridinyl, —N(CH₂OC(O)CH₂CH₃)(C(O)OCH(CH₃)₂), —NHC(O)R₆, azetidinyl, piperidinyl, piperazinyl, morpholinyl, dihydro-1,4-dioxinyl, dihydro-2H-pyranyl, phenyl, imidazolyl, oxazolyl, oxadiazoyl, thiazolyl, and pyridinyl, wherein the azetidinyl, piperidinyl, piperazinyl, morpholinyl, dihydro-1,4-dioxinyl, dihydro-2H-pyranyl, phenyl, imidazolyl, oxazolyl, oxadiazoyl, thiazolyl, or pyridinyl is optionally substituted by 1 to 3 substituents independently selected from hydroxy, halo, C₁₋₄alkyl, C₁₋₄haloalkyl, and —C(O)OCH₃, wherein

R₆ is selected from —OCH₃, —OCH(CH₃)₂, cyclopropyl, cyclobutyl, azetidinyl, pyrrolidinyl, piperidinyl, piperazinyl, oxetanyl, tetrahydrofuranyl, tetrahydropyranyl, morpholinyl, dioxanyl, oxazepanyl, oxabicyclo[2.2.1]heptanyl, bicyclo[2.2.1]hept-2-ene, dihydro-1,4-dioxinyl, phenyl, pyridinyl, imidazolyl, and triazolyl, wherein
the cyclopropyl, cyclobutyl, azetidinyl, pyrrolidinyl, piperidinyl, piperazinyl, oxetanyl, tetrahydrofuranyl, tetrahydropyranyl, morpholinyl, dioxanyl, oxazepanyl, oxabicyclo[2.2.1]heptanyl, bicyclo[2.2.1]hept-2-ene, or dihydro-1,4-dioxinyl of R₆ is optionally substituted by 1 to 3 substituents independently selected from hydroxy, halo, cyano, oxo, methyl, methoxymethyl, methoxy, butoxycarbonyl, amino, and
the phenyl, pyridinyl, imidazolyl, or triazolyl of R₆ is optionally substituted by halo, cyano, methyl, ethyl, isopropyl, —(CH₂)₂OH, —CHF₂, —CF₃, —CH₂CF₃, —(CH₂)₂CF₃, amino, —NHCH₃, —N(CH₃)₂, and butoxycarbonylamino

### Embodiment 7

A compound, or a pharmaceutically acceptable salt, or stereoisomer thereof; according to Embodiment 1, wherein the compound is of Formula A1,

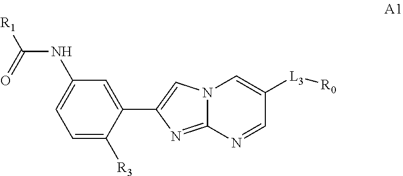

or a pharmaceutically acceptable salt, or stereoisomer thereof; wherein

R₁ is C₄₋₇heterocycloalkyl or C₅₋₆heteroaryl, which of which is optionally substituted by 1-2 substituents independently selected from hydroxy, halo, cyano, oxo, C₁₋₄alkyl, C₁₋₄haloalkyl, C₁₋₆alkoxy, C₁₋₄alkylcarbonyl, aminocarbonyl, C₁₋₄alkylsulfonyl, amino, —NHCH₃, C₃₋₆cycloalkyl, and C₄₋₆heterocycloalkyl;

R₃ is hydrogen or halo;

L₃ is selected from a bond, C₃₋₇cycloalkyl, C₄₋₆heterocycloalkyl, C₅₋₆cycloalkenyl, C₅₋₆heterocycloalkenyl, phenyl, and C₅₋₆heteroaryl;

R₀ is selected from hydrogen, halo, C₁₋₆alkyl, C₁₋₄hydroxyalkyl, C₁₋₄haloalkyl, NR₂ₐR_{2b}, phenyl, and C₅₋₆heteroaryl; provided when L₃ is a bond, R₀ is not hydrogen; wherein
the phenyl or C₅₋₆heteroaryl of R₀ is optionally substituted by 1 to 2 substituents independently selected from halo, C₁₋₄alkyl, C₁₋₄alkyamino, and C₁₋₄alkoxy, and
R₂ₐ and R_{2b} are each independently hydrogen or C₁₋₄alkyl.

### Embodiment 8

A compound, or a pharmaceutically acceptable salt, or stereoisomer thereof; according to any one of Embodiments 1 to 2 and 5 to 7, wherein R₁ is selected from azetidinyl, pyrrolidinyl, piperidinyl, piperazinyl, morpholinyl, tetrahydropyranyl, dioxanyl, pyrrolyl, pyrazolyl, imidazolyl, furanyl, isooxazolyl, oxazolyl, thiazolyl, pyridinyl, and pyrimidinyl, each of which is optional substituted by 1 to 2 substituents independently selected from hydroxy, halo, cyano, oxo, methyl, trifluoromethyl, methoxy, amino, and —NHCH₃.

### Embodiment 9

A compound, or a pharmaceutically acceptable salt, or stereoisomer thereof; according to any one of Embodiments 1 to 2 and 5 to 7, wherein the compound is of Formula A or A1, wherein R₁ is selected from:

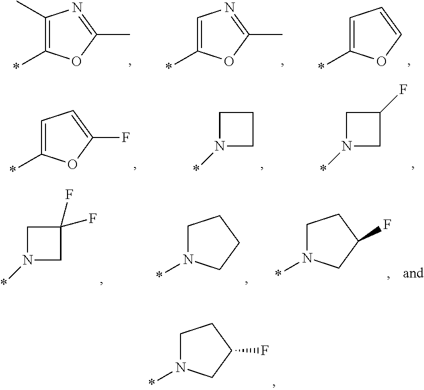

wherein “*” indicates the point of attachment of R₁

### Embodiment 10

A compound, or a pharmaceutically acceptable salt, or stereoisomer thereof, according to any one of Embodiments 1 to 9, wherein the compound is of Formula A or A1, wherein L₃ is selected from C₃₋₇cycloalkyl, C₄₋₆heterocycloalkyl, C₅₋₆cycloalkenyl, C₅₋₆heterocycloalkenyl, phenyl, and C₅₋₆heteroaryl.

### Embodiment 11

A compound, or a pharmaceutically acceptable salt, or stereoisomer thereof, according to any one of Embodiments 1 to 9, wherein the compound is of Formula A or A1, wherein L₃ is selected from cyclopropyl, cyclobutyl, cyclopentyl, bicyclo[3.1.1]heptanyl, pyrrolidinyl, piperidinyl, tetrahydropyranyl, morpholinyl, cyclopentenyl, tetrahydropyridinyl, dihydrofuranyl, dihydropyranyl, phenyl, pyrrolyl, pyrazolyl, imidazolyl, triazolyl, oxazolyl, isooxazolyl, pyridinyl, pyridazinyl, pyrimidinyl, and pyrazinyl.

### Embodiment 12

A compound, or a pharmaceutically acceptable salt, or stereoisomer thereof, according to any one of Embodiments 1 to 9, wherein L₃ is selected from cyclopropyl, cyclobutyl, tetrahydropyranyl, cyclopentenyl, dihydrofuranyl, dihydropyranyl, phenyl, pyrazolyl, imidazolyl, thiazolyl, pyridinyl, and pyrimidinyl.

### Embodiment 13

A compound, or a pharmaceutically acceptable salt, or stereoisomer thereof; according to any one of Embodiments 1 to 4 and 7 to 12, wherein R₀ is selected from hydrogen, halo, methyl, ethyl, isopropyl, isobutyl, —(CH₂)₂OH, —CHF₂, —CF₃, —CH₂CF₃, —(CH₂)₂CF₃, —NHCH₃, phenyl, and pyridinyl, wherein the phenyl or pyridinyl is optionally substituted by 1-2 substituents independently selected from halo and methyl.

### Embodiment 14

A compound, or a pharmaceutically acceptable salt, or stereoisomer thereof, according to any one of Embodiments 1 to 4 and 7 to 9, wherein -L₃-R₀ is selected from methyl, ethyl, isopropyl, —CHF₂, —CF₃, —CH₂CF₃, —(CH₂)₂CF₃,

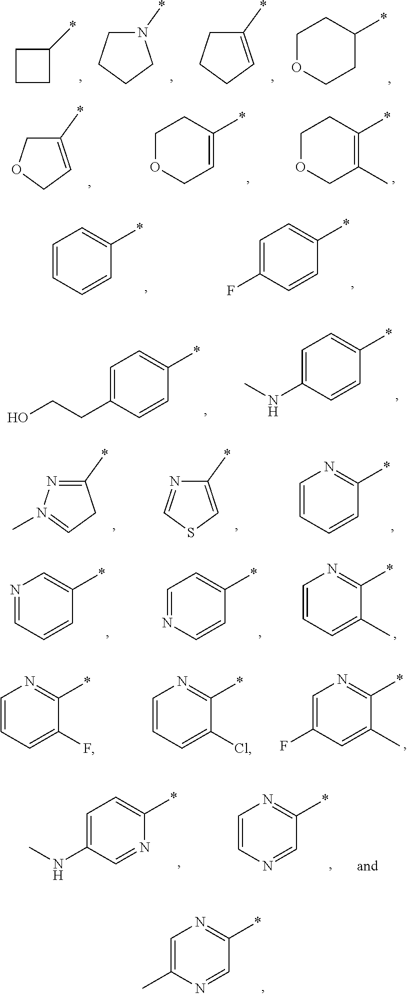

wherein “*” represents the point of attachment of L₃R₀.

### Embodiment 15

A compound, or a pharmaceutically acceptable salt, or stereoisomer thereof, according to Embodiment 1, wherein the compound is of Formula I:

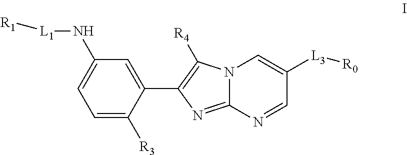

wherein

L₁ is —C(O)— or —S(O)₂—;

R₁ is selected from hydrogen, nitro, C₁₋₄alkyl, C₁₋₆alkoxy, amino, C₅₋₉heteroaryl, C₃₋₆cycloalkyl and C₄₋₆heterocycloalkyl, each of which is optionally substituted by 1-2 substituent independently selected from halo, cyano, amino, C₁₋₄alkyl, haloC₁₋₄alkyl, C₁₋₆alkoxy, and C₁₋₄alkylcarbonyl; or —NHL₁R₁ is nitro;

R₃ is selected from hydrogen, halo, cyano, C₁₋₄alkyl and haloC₁₋₄alkyl;

R₄ is selected from hydrogen, C₁₋₄alkyl, haloC₁₋₄alkyl, and —C(O)R₁₀, wherein R₁₀ is hydroxy, C₁₋₄alkyl, C₁₋₄alkoxy, amino, C₁₋₄alkylamino, C₃₋₆cycloalkyl and C₄₋₅heterocycloalkyl, each of which is independently optionally substituted by 1-2 substituents independently selected from hydroxyl, halo and C₁₋₄alkyl;

L₃ is a bond, phenylene, or C₅₋₆heteroarylene;

R₀ is selected from hydrogen, hydroxyl, halo, nitro, —N═CHN(CH₃)₂, C₁₋₄alkyl, C₁₋₄alkoxy, —NR₂ₐR_{2b}, —NR₅C(O)R₆, —NR₅S(O)₂R₉, C₃₋₆cycloalkyl, C₄₋₆heterocycloalkyl, C₄₋₆heterocycloalkenyl, phenyl and C₅₋₆heteroaryl; wherein
the C₁₋₄alkyl or C₁₋₄alkoxy is optionally substituted by 1-2 substituents independently selected from C₁₋₄alkoxy, amino, phenyl and C₅₋₆heteroaryl; wherein the phenyl or C₅₋₆heteroaryl is optionally substituted by halo or C₁₋₄alkyl;
the C₃₋₆cycloalkyl, C₄₋₆heterocycloalkyl, C₄₋₆heterocycloalkenyl, phenyl and C₅₋₆heteroaryl of R₀ is optionally substituted with halo, oxo, C₁₋₄alkyl, hydroxyC₁₋₄alkYl, haloC₁₋₄alkyl, C₁₋₄alkoxy, amino, C₁₋₄alkylamino, and —(CH₂)₁₋₄NRₐR_{b}, wherein Rₐ and R_{b} are each independently hydrogen, C₁₋₄alkyl or C₃₋₆cycloalkyl;
R₂ₐ is hydrogen or C₁₋₄alkyl;
R_{2b} is selected from hydrogen, C₁₋₄alkyl, wherein the alkyl is optionally substituted by amino, C₄₋₆heterocycloalkyl, phenyl or C₅₋₆heteroaryl, wherein the C₄₋₆heterocycloalkyl, phenyl or C₅₋₆heteroaryl is optionally substituted by hydroxyl, halo or C₁₋₄alkyl;
R₅ is hydrogen or C₁₋₄alkyl;
R₆ is selected from hydrogen, C₁₋₆alkyl, C₁₋₄alkoxy, C₃₋₆cycloalkoxy, amino, C₃₋₆cycloalkyl, C₅₋₆heterocycloalkyl, and C₅₋₆heteroaryl, wherein
   the C₁₋₄alkyl, C₁₋₄alkoxy, C₃₋₆cycloalkoxy, and amino of R₆ are each optionally substituted by 1-2 substituents independently selected from halo, hydroxy, C₁₋₄alkyl, haloC₁₋₄alkyl, C₁₋₄alkoxy, —NR₉ₐR_{9b}, C₃₋₆cycloalkyl, C₅₋₆heterocycloalkyl, and C₅₋₆heteroaryl, wherein R₉ₐ is hydrogen or C₁₋₄alkyl and R_{9b} is selected from hydrogen, C₁₋₄alkyl, C₁₋₄alkylcarbonyl and C₁₋₄alkoxycarbonyl, and the C₅₋₆heterocycloalkyl and C₅₋₆heteroaryl are each optionally substituted by 1-2 substituents independently selected from hydroxyl, C₁₋₄alkyl and C₁₋₄alkoxycarbonyl,
   the C₅₋₆heteroaryl of R₆ is optionally substituted with 1-2 substituents selected from hydroxy, C₁₋₄alkyl, and C₁₋₄alkoxycarbonyl,
   the C₃₋₆cycloalkyl or C₄₋₆heterocycloalkyl of R₆ are each independently optionally substituted by 1-2 substituents independently selected from halo, cyano, hydroxy, C₁₋₄alkyl, haloC₁₋₄alkyl, C₁₋₄alkoxylC₁₋₄alkyl, aminocarbonyl, C₁₋₄alkoxycarbonyl, and C₁₋₄alkoxycarbonylaminoC₁₋₄alkyl, and
   R₈ is C₁₋₄alkyl or C₁₋₄alkylamino.

### Embodiment 16

A compound of Formula I, or a pharmaceutically acceptable salt, or stereoisomer thereof; according to Embodiment 15, wherein L₁-R₁ is selected from

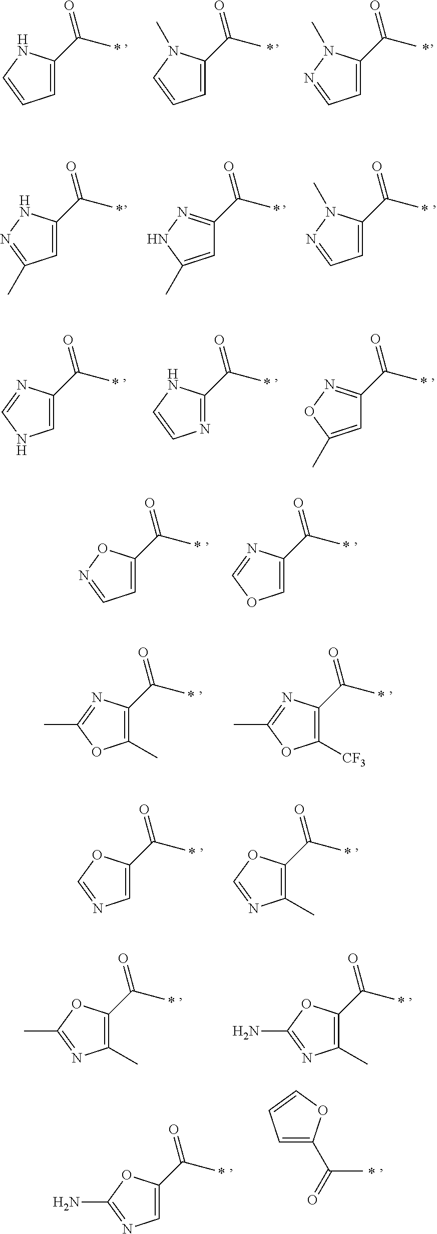

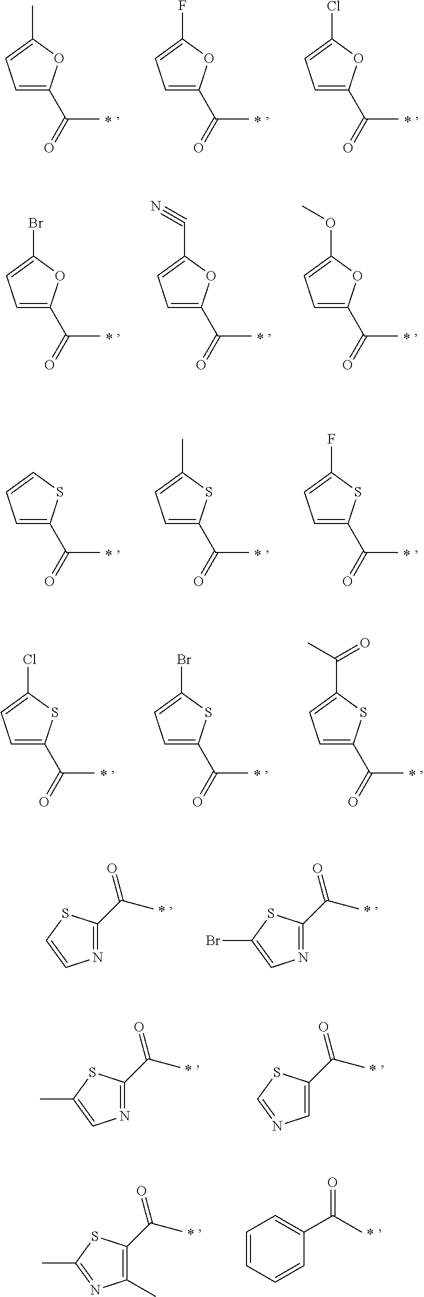

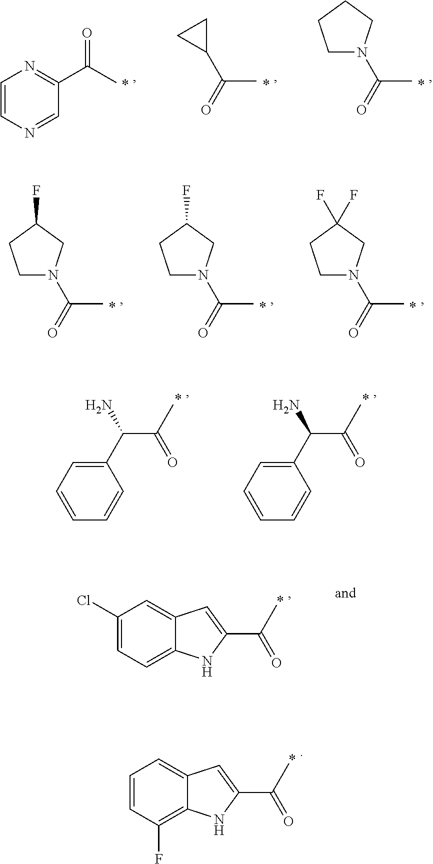

### Embodiment 17

A compound of Formula I, or a pharmaceutically acceptable salt, or stereoisomer thereof; according to Embodiment 15 or 16, wherein R₃ is chloro or fluoro,

### Embodiment 18

A compound of Formula I, or a pharmaceutically acceptable salt, or stereoisomer thereof, according to any one of Embodiments 15 to 17, wherein R₄ is hydrogen.

### Embodiment 19

A compound of Formula I, or a pharmaceutically acceptable salt, or stereoisomer thereof, according to any one of Embodiments 15 to 18, wherein R₀ is selected from hydrogen, halo, nitro, hydroxyl, C₁₋₄alkoxy, amino, C₁₋₄alkylamino, —NH(CH₂)₁₋₂-phenyl, —NR₅C(O)R₆, —NR₀S(O)₂R₈, oxazolidin-2-one, 1,2,4-triazol-5(4H)-one, pyrrolidin-2-one, phenyl and C₅₋₆heteroaryl; wherein

the oxazolidin-2-one, 1,2,4-triazol-5(4H)-one, pyrrolidin-2-one, phenyl or C₅₋₆heteroaryl is optionally substituted with halo, C₁₋₄alkyl, C₁₋₄alkoxy, amino, C₁₋₄alkylamino, (CH₂)₁₋₄OH, and (CH₂)₁₋₄NRₐR_{b}, wherein Rₐ and R_{b} are each independently hydrogen, C₁₋₄alkyl or C₃₋₆cycloalkyl;

R₅ is hydrogen or C₁₋₄alkyl;

R₆ is selected from C₁₋₆alkyl, C₁₋₆alkoxyl, C₃₋₆cycloalkyl, C₅₋₆heterocycloalkyl, and C₅₋₆heteroaryl, each of which is optionally substituted with 1 to 2 substituents independently selected from hydroxyl, C₁₋₄alkoxy, amino, C₁₋₄alkylamino; and

R₈ is C₁₋₄alkyl or C₁₋₄alkylamino.

### Embodiment 20

A compound of Formula A, or a pharmaceutically acceptable salt, or stereoisomer thereof, according to Embodiment 1, wherein the compound is selected from the exemplified compounds listed in Table II, infra (pages 232-354), wherein the corresponding chemical names of said compounds are listed on pages 45 to 70, supra, of the specification.

### Embodiment 21

A pharmaceutical composition comprising a compound according to any one of Embodiments 1 to 20 as an active ingredient and at least one excipient.

### Embodiment 23

A method for treating, preventing, inhibiting, ameliorating, or eradicating the pathology and/or symptomology of a disease caused by a parasite, comprising administering to a subject a therapeutically effective amount of a compound according to any one of Embodiments 1 to 20 or a composition according to Embodiment 21, wherein the disease is selected from Leishmaniasis, Human African Trypanosomiasis, and Chagas disease, and wherein the administering is optionally in combination with a second agent.

As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)—.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by Plasdmodium or (ii) associated with Plasdmodium activity, or (iii) characterized by activity (normal or abnormal) of Plasdmodium or (2) reduce or inhibit the activity of Plasdmodium; or (3) reduce or inhibit the growth of Plasdmodium. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of Plasdmodium; or at least partially reducing or inhibiting the growth of Plasdmodium.

As used herein, the term “subject” refers to an animal. Typically the animal is a mammal. A subJect also refers to for example, primates (e.g., humans, male or female), cows, sheep, goats, horses, dogs, cats, rabbits, rats, mice, fish, birds and the like. In certain embodiments, the subJect is a primate. In yet other embodiments, the subJect is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subJect is “in need of” a treatment if such subJect would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

### General Processes for Preparing Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Synthesis”, John Wiley and Sons, 1991.

Typically, the compounds of formula (I) can be prepared according to Schemes 1A, 2 and 3 provided infra., where the variables: R₀, R₁, R₃, R₄, R₇ and others are as defined in the Summary of the Invention. The following reaction schemes are given to be illustrative, not limiting, descriptions of the synthesis of compounds of the invention. Detailed descriptions of the synthesis of compounds of the Invention are given in the Examples, infra.

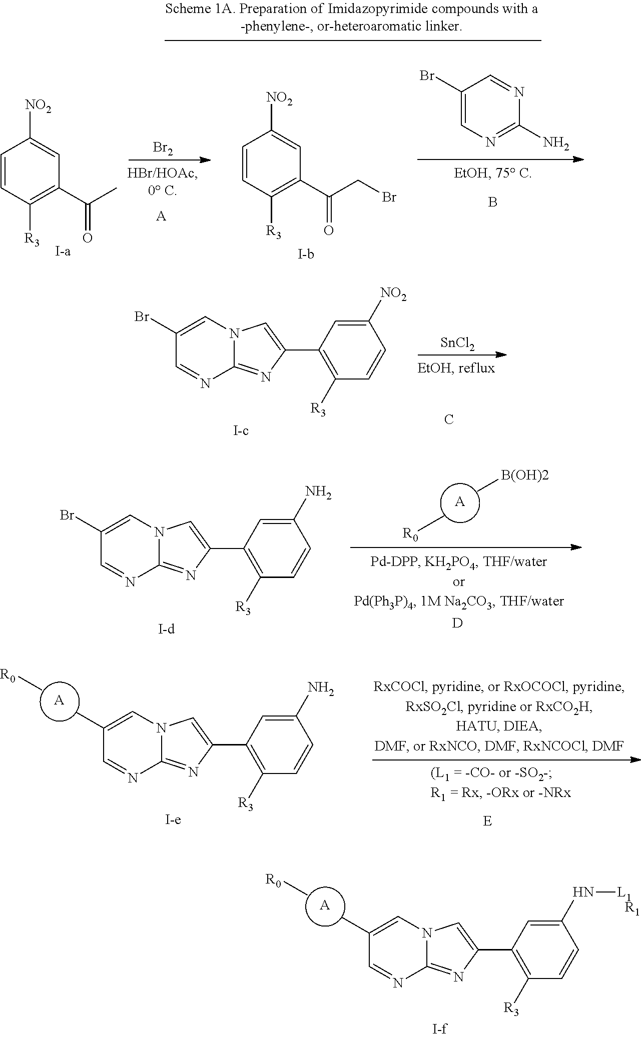

The Imidazopyrimidine analog having a *-phenylene- or *-heteroaaromatic linker (1f) can be synthesized according to Scheme 1A.
A. Bromination of α-alkyl ketone with bromine in acetic acid;
B. Condensation of 1 b with 5-Bromopyrimidine amine with ethanol at elevated temperatures in the presence or absence of acid leading to formation of imidazopyrimidine 1c;
C. Reduction of nitro to the corresponding amine using tin chloride and ethanol as solvent. This reduction can also be achieved by other metals such as Fe, HCl or zinc. Alternatively, this reduction can be carried out by catalytic hydrogenation using Pd, Pt, or Raney-Ni using hydrogen gas as atmosphere;
D. Suzuki coupling; and
E. Coupling of the amine to form an amide either by using an acid chloride in pyridine as solvent or direct coupling of carboxylic acid using a suitable peptide coupling agent such as HATU in presence of amine base such as Et₃N, DIEA (Hunig's base). Carbamates can be formed either by reaction with RₓOCOCl or activated carbonates such as 4-nitophenyl activated carbonates in the presence of base such as pyridine or DIEA. Sufonamides are formed by the reaction of sulfonyl chloride with aniline in the presence of base such as pyridine. Ureas are formed by reaction of amine with isocyanate or carbamoyl chloride in DMF with or without base.

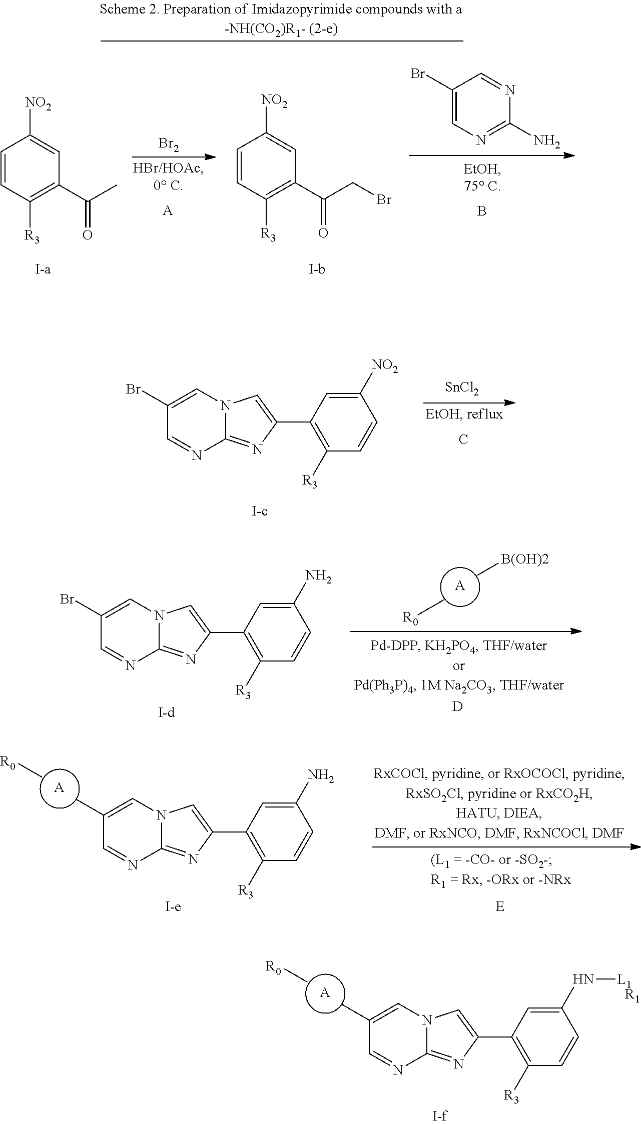

A. Reduction of nitro group using Pd—C in hydrogen atmosphere;
B. Formation of carbamate by reaction with chloroformate or carbonic anhydride. Alternatively, amides can be formed by reaction with acid chloride or carboxylic acid in the presence of coupling agent;
C. Condensation of ketobromide (1-c) with 2-b with acetone at elevated temperatures in the presence or absence of acid leading to formation of imidazopyrimidine 2-c;
D. Reduction of nitro reduction to the corresponding amine using tin chloride and ethanol as solvent. This reduction can also be achieved by other metals such as Fe, HCl o zinc. Alternatively, this reduction can be carried out by catalytic hydrogentation using Pd, Pt, or raney-Ni using hydrogen gas as atmosphere; and
E. Coupling of the amine to form a amide either by using an acid chloride in pyridine as solvent or direct coupling of carboxylic acid using a suitable peptide coupling agent such as HATU in presence of amine base such as Et₃N, DIEA (Hunig's base). Carbamates can be formed either by reaction with RₓOCOCl or activated carbonates such as 4-nitophenyl activated carbonates in the presence of base such as pyridine or DIEA. Sufonamides are formed by the reaction of sulfonyl chloride with aniline in the presence of base such as pyridine. Ureas are formed by reaction of amine with isocyanate or carbamoyl chloride in DMF with or without base.

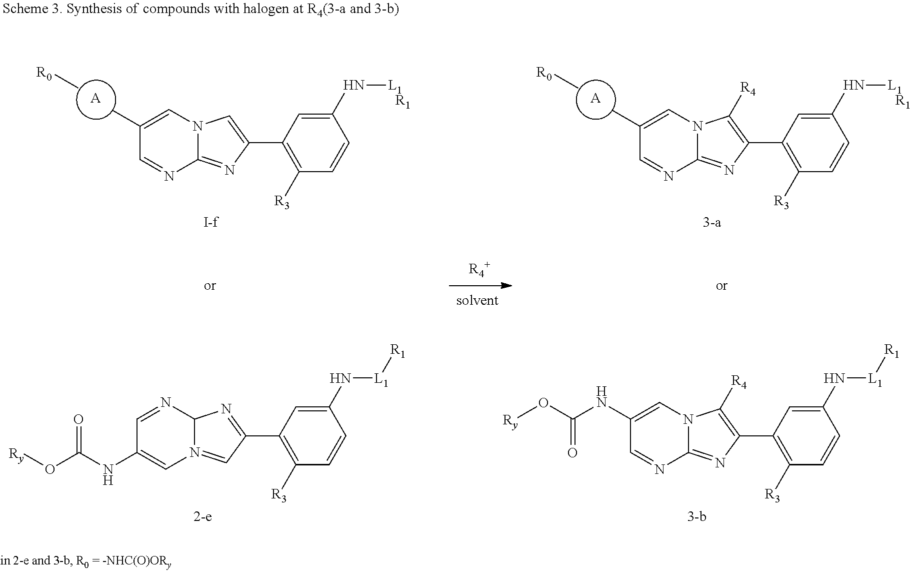

A. Introduction of halogen (F, Cl, Br or I) by reacting with suitable halogenating agents such as Selectfluor, N-chlorosuccinimide, N-bromosuccinimide and N-iodosuccinimide or any halogenation agents in the presence of solvents such as chloroform, dichloromethane, acetic acid and acetonitrile.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

Compounds of the invention and intermediates can also be converted into each other according to methods generally known to those skilled in the art.

Compounds of the invention are useful in the treatment and/or prevention of infections such as Leishmaniasis, Human African Trypanosomiasis, or Chagas disease.

Leishmaniasis is a disease caused by protozoan parasites that belong to the genus Leishmania infantum, Leishmania braziliensis, Leishmania panamensis, Leishmania guayanensis, Leishmania amazonensis, Leishmania mexicana, Leishmania tropica, or Leishmania maJor, and more typically caused by Leishmania donovani. These parasites are typically transmitted by the bite of an infected female sandfly from Phlebotomus or Lutzomyia genus.

Leishmaniasis is mostly a disease of the developing world, and is rarely known in the developed world outside a small number of cases, mostly in instances where troops are stationed away from their home countries. Leishmaniasis can be transmitted in many tropical and subtropical countries, and is found in parts of about 88 countries. Approximately 350 million people live in these areas. The settings in which leishmaniasis is found range from rainforests in Central and South America to deserts in West Asia and the Middle East. It affects as many as 12 million people worldwide, with 1.5-2 million new cases each year.^{[19]} The visceral form of leishmaniasis has an estimated incidence of 500,000 new cases and 60,000 deaths each year. More than 90 percent of the world's cases of visceral leishmaniasis are in India, Bangladesh, Nepal, Sudan, and Brazil. Kabul is estimated as the largest center of cutaneous leishmaniasis in the world, with approximately 67,500 cases as of 2004.

There are four main forms of Leishmaniasis. Cutaneous leishmaniasis is the most common form of leishmaniasis. Visceral leishmaniasis, also called kala-azar, is the most serious form in which the parasites migrate to the vital organs. Visceral leishmaniasis is caused by the parasite Leishmania donovani, and is potentially fatal if untreated.

Currently, no vaccines are in routine use. The two main therapies for visceral leishmaniasis are the antimony derivatives sodium stibogluconate (Pentostam®) and meglumine antimoniate (Glucantim®). Sodium stibogluconate has been used for about 70 years and resistance to this drug is a growing problem. In addition, the treatment is relatively long and painful, and can cause undesirable side effects. Amphotericin (AmBisome) is now the treatment of choice. Miltefosine (Impavido), and paromomycin are the other treatment alternatives. These drugs are known to produce a definitive cure in >90% of patients. Amphotericin (AmBisome) is expansive and has to be given intravenously; it is not affordable to most patients affected. Paromomycin, although affordable, requires intramuscular injections for 3 weeks; compliance is a maJor issue. Miltefosine is an oral drug and has shown to be more effective and better tolerated than other drugs. However, there are problems associated with the use of miltefosine that arise from its teratogenicity and pharmacokinetics. Miltefosine was shown to be much slower eliminated from the body and was still detectable five months after the end of treatment. The presence of subtherapeutic miltefosine concentrations in the blood beyond five months after treatment might contribute to the selection of resistant parasites and, moreover, the measures for preventing the teratogenic risks of miltefosine must be reconsidered. This led to some reluctance to taking Miltefosine by affected populations.

The Drugs for Neglected Diseases Initiative is actively facilitating the search for novel therapeutics. Our invention meets that need.

Human African trypanosomiasis (HAT), also known as African sleeping sickness, is a vector-borne parasitic disease caused by the protozoa Trypanosoma brucei. There are two subspecies that infect humans, Tb. gambiense and Tb. rhodesiense, with the former accounting for over 95% of reported cases and the latter accounting for the remaining reported cases. The parasites are transmitted to humans by tsetse fly (Glossina genus) bites which have acquired their infection from human beings or from animals harboring the human pathogenic parasites.

The disease has been recorded as occurring in 36 countries, all in subtropical and equatorial Africa. It is endemic in southeast Uganda and western Kenya. In 1995, the WHO estimated that 300,000 people were afflicted with the disease. In its 2001 report, the WHO set the figure of people at risk of infection at 60 million, of which only 4 to 5 million had access to any kind of medical monitoring. In 2006, the WHO estimated that about 70,000 people could have the disease, and many cases are believed to go unreported. About 48,000 people died of sleeping sickness in 2008. Public health efforts in prevention and the eradication of the tsetse fly population have proven to be successful in controlling the spread of the disease; under 10,000 new cases were reported in 2009 according to WHO figures, which represents a huge decrease from the estimated 300,000 new cases in 1998.

African trypanosomiasis symptoms occur in two stages. In the first stage, known as the haemolymphatic phase, the trypanosomes multiply in subcutaneous tissues, blood and lymph. The haemolymphatic phase is characterized by bouts of fever, headaches, Joint pains and itching. In the second stage, the neurological phase, the parasites cross the blood-brain barrier to infect the central nervous system. It is in this stage when more obvious signs and symptoms of the disease appear: changes of behaviour, confusion, sensory disturbances and poor coordination. Disturbance of the sleep cycle, which gives the disease its name, is an important feature of the second stage of the disease. Without treatment, the disease is invariably fatal, with progressive mental deterioration leading to coma, systemic organ failure, and death.

Four drugs are registered for the treatment of sleeping sickness. The protocol depends on the stage of the disease. The current standard treatment for first-stage disease is intravenous or intramuscular pentamidine (for T.b. gambiense), or intravenous suramin (for T.b. rhodesiense). The current standard treatment for second-stage disease is: Intravenous melarsoprol, or interavenous melarsoprol in combination with oral nifurtimox, intravenous eflornithine only or eflornithine in combination with nifurtimox. All of the drugs have undesirable or sometime serious side effects. For example, 3-10% of patients those injected with Melarsoprol (Arsobal), an organoarsenical, developed reactive encephalopathy (convulsions, progressive coma, or psychotic reactions), and 10-70% of such cases result in death. There remains a need for new therapy.

Chagas disease, also called American trypanosomiasis, is a tropical parasitic disease caused by the flagellate protozoan Trypanosoma cruzi. T. cruzi is commonly transmitted to humans and other mammals by the blood-sucking “kissing bugs” of the subfamily Triatominae (family Reduviidae).

Chagas disease is contracted primarily in the Americas. It is endemic in twenty one Central and Latin American countries; particularly in poor, rural areas of Mexico, Central America, and South America. Large-scale population movements from rural to urban areas of Latin America and to other regions of the world have increased the geographic distribution of Chagas disease, and cases have been noted in many countries, particularly in Europe. Rarely, the disease has been found in the Southern part of the United States.

Each year, an estimated 10 to 15 million people across the world are infected with Chagas disease, most of whom do not know they are infected. Every year, 14,000 people die as a consequence of the disease. In Central and South America, Chagas kills more people than any other parasite-borne disease, including malaria. By applying published seroprevalence figures to immigrant populations, CDC estimates that more than 300,000 persons with Trypanosoma cruzi infection live in the United States. Most people with Chagas disease in the United States acquired their infections in endemic countries.

Chagas disease has an acute and a chronic phase. If untreated, infection is lifelong. Acute Chagas disease occurs immediately after infection, may last up to a few weeks or months, and parasites may be found in the circulating blood. Infection may be mild or asymptomatic. There may be fever or swelling around the site of inoculation (where the parasite entered into the skin or mucous membrane). Rarely, acute infection may result in severe inflammation of the heart muscle or the brain and lining around the brain. The initial acute phase is responsive to antiparasitic treatments, with 60-90% cure rates. Following the acute phase, most infected people enter into a prolonged asymptomatic form of disease (called “chronic indeterminate”) during which few or no parasites are found in the blood. During this time, most people are unaware of their infection. Many people may remain asymptomatic for life and never develop Chagas-related symptoms. However, an estimated 20-30% of infected people will develop debilitating and sometimes life-threatening medical problems over the course of their lives.

The symptoms of Chagas disease vary over the course of an infection. In the early, acute stage, symptoms are mild and usually produce no more than local swelling at the site of infection. The initial acute phase is responsive to antiparasitic treatments, with 60-90% cure rates. After 4-8 weeks, individuals with active infections enter the chronic phase of Chagas disease that is asymptomatic for 60-80% of chronically infected individuals through their lifetime.

There is no vaccine against Chagas disease. Treatment for Chagas disease focuses on killing the parasite and managing signs and symptoms.

During the acute phase of Chagas disease, the drugs currently available for treatment are benznidazole and nifurtimox. Once Chagas disease reaches the chronic phase, medications aren't effective for curing the disease. Instead, treatment depends on the specific signs and symptoms. However, problems with these current therapies include their diverse side effects, the length of treatment, and the requirement for medical supervision during treatment. Resistance to the two frontline drugs has already occurred. The antifungal agent Amphotericin b has been proposed as a second-line drug, but this drug is costly and relatively toxic.

In accordance with the foregoing, the present invention further provides a method for preventing or treating Leishmaniasis, Chaga disease or Human African Trypanosomiasis in a subJect in need of such treatment, which method comprises administering to said subJect a therapeutically effective amount of a compound selected from Formula I or a pharmaceutically acceptable salt thereof. The required dosage will vary depending on the mode of administration, the particular condition to be treated and the effect desired.

In one embodiment of the method of the invention, the disease being treated is Leishmaniasis caused by the parasites Leishmania donovani, Leishmania infantum, Leishmania braziliensis, Leishmania panamensis, Leishmania guayanensis, Leishmania amazonensis, Leishmania mexicana, Leishmania tropica, Leishmania major. In one variation of the above embodiment, the disease being treated is Visceral leishmaniasis, caused by the parasite Leishmania donovani.

In another embodiment of the method of the invention, the disease being treated is Human African Trypanosomiasis caused by a protozoa belonging to the species Trypanosoma brucei. In one embodiment, the protozoa is Trypanosoma brucei gambiense. In another embodiment, the protozoa is Trypanosoma brucei rhodesiense.

In yet another embodiment of the method of the invention, the disease being treated is Chagas disease (also call American Trypanosomiasis) caused by protozoa Trypanosoma cruzi.

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). In one embodiment, the compound of the invention is administered with the known treatment drugs. For example, for the treatment of Leishmaniasis, compound of the invention may be used in combination with stibogluconate, meglumine antimoniate, Amphotericin, Miltefosine and paromomycin. For the treatment of Human African Trypanosomiasis, the compound of the invention may be used in combination with pentamidine, suramin, melarsoprol, eflornithine, and nifurtimox. For the treatment of Chagas disease, the compound of the invention may be used in combination with benznidazole, nifurtimox and Amphotericin.

Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) optionally co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Biological Assays

Leishmania donovani Axenic Amastigote Proliferation Assay

Leishmania donovani axenic amastigote parasites are grown at 37° C., 5% CO₂ in media made of RPMI 1640, 4 mM L-glutamine, 20% heat inactivated FBS, 100 units/mL of penicillin and 100 μg/mL of streptomycin, 23 μM Folic Acid, 100 μM Adenosine, 22 mM D-glucose, 25 mM MES. The pH of media is adjusted to 5.5 at 37° C. using HCl. 20 μL of media is first dispensed into 384 well plates and 100 nL of the compounds of invention in DMSO are added to the plate wells. At the same time control compounds and DMSO are added to plates to serve as the positive and negative controls, respectively. 40 μL of parasite culture (9600 parasites) are then added to the plate wells. The plates are then placed into incubators. After 2 day incubation, 20 μL of Cell TiterGlo (Promega) is added to the plate wells. The luminescence signal of each well is measured using the Envision reader (Perkin Elmer). The percentage inhibition of 50%, EC₅₀, is calculated for each of the compounds.

Compounds of the invention have an EC₅₀ of 25 μM or less, typically less than 5 μm, and about half of the compounds have an EC₅₀ below 1 μM. Selected compounds of the invention can significantly delay the proliferation of L. donovani. The inhibitory efficacy of the compounds of the invention against L. donovani axenic amastigotes in vitro is provided in Table I.

### Assay for Proliferation of Leishmania Parasites

The activity of a compound according to the present invention for inhibition of parasitemia can be assessed by the parasite proliferation assay. The assay measures the increase in the parasite number in the assayed plate well using a DNA intercalating dye, SYBR Green I® dye (INVITROGEN) to stain Leishmania cell nuclei. It is understood that the assays illustrate the invention without in any way limiting the scope of the invention.

L. donovani HU3 strain is propagated by infecting BALB/c mice through tail vein injection with 10⁷ Leishmania parasites. Infected mice are allowed to develop infection during 9-11 weeks post-infection. During this time, the parasites accumulate in the infected mouse spleens to large numbers, and the infected mice serve as the source of parasites for the in vitro measurement of compound efficacies. To assay a compound for anti-leishmanial activity, peritoneal macrophages isolated from non-infected BALB/c mice are seeded into 384-well plates at density 2×10⁴ macrophages per well in 25 mL of medium (RPMI1640, 10% fetal serum albumin, 10 mM HEPES, 1 mM sodium pyruvate, 1% Pen/Strep). Subsequently, the seeded plates are placed into an incubator set to maintain 37° C. temperature and atmosphere with 5% CO₂. The next day, Leishmania parasites are isolated from the spleens of mice infected for 9-11 weeks and 4×10⁵ isolated parasites in 10 mL of the above media are added to each plate well. Plates are then returned into incubators and infection is allowed to proceed for 24 hours. After the infection of macrophages is completed, 5 mL of compounds of the invention in the above medium, which also contains 5% DMSO, are added to plate wells containing infected macrophages. At the same time control compounds (miltefosine and amphotericin B) and DMSO are added to plates to serve as the positive and negative controls, respectively. After the compound addition, the plates are returned into incubator and cells infected with parasites are cultured for 5 days. At the end of cultivation, 40 mL of 8% paraformaldehyde is added to plate wells and incubated for 15 min at room temperature. Following the incubation, the paraformaldehyde from plate wells is aspirated, and 40 mL of PBS containing 0.2% Triton X-100 is added to wells. After 15 min incubation, the solution is aspirated from wells again, and replaced with SybrGreen Dye solution in PBS (1:125,000 dilution). Infected cells are imaged with Evotec Opera high-content microscope, and the [symbol] staining with SybrGreen dye. The percentage inhibition of 50%, EC₅₀, is calculated for each compound.

Compounds of the invention have an EC₅₀ of typically less than 10 μM; about 50% of the compounds analyzed have an EC₅₀ of less than 1 μm. Selected compounds have EC50 less than 200 nM. The data shows the compounds of the invention can significantly delay the proliferation of L. donovani.

The inhibitory efficacy of the compounds of the invention against proliferation of L. donovani in mouse peritoneal macrophages is provided in Table I.

Assay for Proliferation of Kinetoplastid Parasite Trypanosoma brucei.

The proliferation is quantified by the addition of Cell Titer Glo (Promega®) a luminescent cell viability assay that measures the number of viable cells in culture based on the quantification of cellular ATP amount, which is an indicator of metabolically active cells.

The following assay illustrates the invention without in any way limiting the scope of the invention. This parasite proliferation assay measures the increase in parasite growth using an assay that measures ATP activity, Cell Titer Glo®.

Trypanosoma brucei Lister 427 strain is grown in HMI-9 Trypanosome media for T. brucei bloodstream form. 30 μl of HMI-9 media is dispensed into 384 well assay plates. 200 nl of compounds of the invention (in DMSO), including anti-trypanosome controls (Pentamidine and suramin), are then transferred into the assay plates, as well as DMSO alone to serve as a negative control for inhibition. Then 25 μl of a suspension of T. brucei culture in HMI-9 media is dispensed into the assay plates. The final concentration of parasites in culture corresponds to 1.7% of 0.5 uM ATP activity with Cell Titer Glo® in HMI-9 media. The plates are placed in a 37° C. incubator for 48 hours in an atmospheric environment containing 5% CO₂. 40 μl of Cell Titer Glo® is dispensed into the plates. The plates are then read for luminescence. The percentage inhibition of 50%, EC₅₀, is calculated for each compound.

Compounds of the invention have an EC₅₀ of 5 μM or less, typically less than 1 μM, and more typically less than 200 nM. The data supports that compounds of the invention can significantly delay the proliferation of T. brucei.

The inhibitory efficacy of the compounds of the invention against the proliferation of T. brucei is provided in Table I, infra.

Assay for Proliferation of Kinetoplastid Parasite Trypanosoma cruzi.

Compounds of the invention can be assayed to measure their capacity to inhibit proliferation of kinetoplastid parasite Trypanosoma cruzi. The screening procedure is for identifying compounds with inhibitor activity against Trypanosoma cruzi amastigotes cultured in 3T3 fibroblast cells. The assay is done using the mammalian stage (amastigotes) of T. cruzi that replicates in the intracellular space of host cells. The host cells are initially infected with the culture-derived trypomastigotes that rapidly invade and then divide as amastigotes. The protocol uses the Tulahuen strain of T. cruzi that has been engineered to express the E. coli beta-galactosidase gene (Lac-Z) (Antimicr. Agents Chemoth. 40:2592, 1996). This allows for a colorimetric readout by using the substrate CPRG and an absorbance plate reader.

3T3 fibroblast cells are re-suspended in RPMI-1640 medium without phenol red medium supplemented with 10% FBS (heat inactivated), 100 μg/mL penicillin, and 100 μg/mL streptomycin. Forty μL of suspension (1,000 cells) is dispensed into 384-well plates and incubated overnight at 37° C. temperature and in atmosphere containing 5% CO₂. The following day, 100 nL of compounds of the invention in DMSO are added to plate wells containing 3T3 cells. At the same time control compounds (benznidazole and nifurtimox) and DMSO are added to plates to serve as the positive and negative controls, respectively. After that, 10 μL of media containing 10,000 T. cruzi trypomastigotes are added to each plate well and plates are placed back into incubators. After 6 day incubation, 10 μL of reagent solution (0.6 mM CPRG, 0.6% NP-40 in PBS) is added to plates and incubated at room temperature for 2 hours. Absorbance is then measured on SpectraMax Gemini fluorimeter to determine relative number of T. cruzi cells present in each plate well. The percentage inhibition of 50%, EC₅₀, is calculated for each compound. Of the three compounds assayed, the EC50s are at the sub-micromolar level. Compound 1, Propan-2-yl N-{2-[2-chloro-5-(5-fluorofuran-2-amido)phenyl]imidazo[1,2-a]pyrimidin-6-yl}carbamate, has an EC50 of 30 nM; Compound 2, propan-2-yl N-(2-{2-chloro-5-[(pyrrolidin-1-yl)carbonylamino]phenyl}imidazo[1,2-a]pyrimidin-6-yl)carbamate, has an EC50 of 243 nM; and Compound 3, propan-2-yl N-{2-[3-(5-fluorofuran-2-amido)phenyl]imidazo[1,2-a]pyrimidin-6-yl}carbamate, has an EC50 of 510 nM. The data shows selected compounds of the invention can significantly delay the proliferation of T. cruzi. The inhibitory efficacy of the compounds of the invention against proliferation of T. cruzi is reported in Table I below.

TABLE I
Inhibitory efficacy of selected compounds of the invention against
proliferation of L. donovani, T cruzi or T. brucei

| | L. donovani | L. donovani | | |
|---|---|---|---|---|
| Example | amastigotes | Macrophage | T cruzi | T. brucei |
| No. | EC50 (μM) | EC50 (μM) | EC50 (μM) | EC50 (μM) |
| 1 | 0.088 | 0.243 | 0.03 | n.d. |
| 2 | 0.096 | 0.268 | 0.243 | n.d. |
| 3 | 0.24 | 0.43 | 0.51 | n.d. |
| 4 | 1.34 | 2.22 | n.d. | 2.363 |
| 5 | 1.08 | 4 | n.d. | n.d. |
| 6 | 22.3 | 9.2 | n.d. | 0.6645 |
| 7 | 2.53 | 1.86 | n.d. | n.d. |
| 8 | 1.59 | 4.7 | n.d. | n.d. |
| 9 | 2.48 | 1.32 | n.d. | 0.1913 |
| 10 | 2.98 | 2.36 | n.d. | 0.2309 |
| 11 | 3.8 | 5 | n.d. | 0.6594 |
| 12 | n.d. | n.d. | n.d. | 0.06557 |
| 13 | 1.72 | 0.81 | n.d. | 0.1806 |
| 14 | 4.2 | 3.2 | n.d. | 0.4787 |
| 15 | 1.5 | 2.95 | n.d. | n.d. |
| 16 | >25 | 14 | n.d. | 1.64 |
| 17 | 0.41 | 0.35 | n.d. | n.d. |
| 18 | 0.41 | 0.172 | n.d. | 0.05679 |
| 19 | 19.1 | 46.9 | n.d. | n.d. |
| 20 | 13.4 | 7.9 | n.d. | 2.002 |
| 21 | 1.97 | 1.05 | n.d. | 0.1867 |
| 22 | 1.24 | 0.53 | n.d. | 0.3843 |
| 23 | 7.3 | 24.7 | n.d. | n.d. |
| 24 | 0.0199 | 0.057 | n.d. | 0.008451 |
| 25 | 0.054 | 0.1 | n.d. | 0.02753 |
| 26 | 0.057 | 0.104 | n.d. | 0.02335 |
| 27 | 0.035 | 0.11 | n.d. | 0.589 |
| 28 | 0.028 | 0.129 | n.d. | 0.009364 |
| 29 | 0.036 | 0.152 | n.d. | n.d. |
| 30 | 0.101 | 0.172 | n.d. | 0.08677 |
| 31 | 0.169 | 0.243 | n.d. | 0.06766 |
| 32 | 0.118 | 0.33 | n.d. | 0.1863 |
| 33 | 0.37 | 0.66 | n.d. | 0.1883 |
| 34 | 0.258 | 0.75 | n.d. | 0.4925 |
| 35 | 0.73 | 0.87 | n.d. | 1.121 |
| 36 | 0.56 | 1.28 | n.d. | n.d. |
| 37 | 0.61 | 3.01 | n.d. | 0.1966 |
| 38 | 5.9 | 15.6 | n.d. | 1.803 |
| 39 | >3.4 | 0.18 | n.d. | 0.07449 |
| 40 | n.d. | n.d. | n.d. | 0.185 |
| 41 | n.d. | n.d. | n.d. | 4.396 |
| 42 | n.d. | n.d. | n.d. | 1.697 |
| 43 | 0.782 | 3.91 | 0.409 | 0.474 |
| 44 | 0.121 | 0.193 | 0.047 | 0.039 |
| 45 | 0.226 | 0.452 | 0.4 | 0.522 |
| 46 | 1.388 | 4.99 | 0.262 | 3.008 |
| 47 | 0.077 | 0.22 | 0.442 | 0.128 |
| 48 | 0.082 | 0.139 | 0.157 | n.d. |
| 49 | >25 | >50 | >20 | >50 |
| 50 | >25 | >50 | >20 | >50 |
| 51 | 3.8 | 9.33 | 1.491 | n.d. |
| 52 | 0.261 | 2.745 | 1.284 | 0.571 |
| 53 | 0.448 | 6.27 | 0.572 | 0.386 |
| 54 | 1.972 | 14.47 | 1.292 | n.d. |
| 55 | 7.23 | 27.08 | 4.14 | n.d. |
| 56 | 19.25 | >50 | 11.53 | n.d. |
| 57 | 0.861 | 2.941 | 0.411 | n.d. |
| 58 | 14.24 | 10.49 | 4.53 | n.d. |
| 59 | >25 | >50 | >20 | n.d. |
| 60 | 0.727 | 1.102 | 0.444 | 1.543 |
| 61 | 0.245 | 0.715 | n.d. | n.d. |
| 62 | 1.999 | 11.55 | n.d. | n.d. |
| 63 | 0.089 | 0.222 | 0.124 | n.d. |
| 64 | 0.161 | 0.469 | n.d. | n.d. |
| 65 | 2.038 | 9.13 | n.d. | n.d. |
| 66 | 2.806 | 16.15 | 0.444 | 1.841 |
| 67 | 0.677 | 1.126 | 0.136 | n.d. |
| 68 | 14.21 | 39.25 | 3.6 | 14.25 |
| 69 | 1.404 | 3.3 | 0.403 | 3.115 |
| 70 | 0.136 | 0.19 | 0.072 | 0.054 |
| 71 | 1.891 | 3.23 | n.d. | n.d. |
| 72 | 0.764 | 8.39 | n.d. | n.d. |
| 73 | 1.288 | 15.19 | n.d. | n.d. |
| 74 | 0.608 | 3.89 | 0.608 | 0.49 |
| 75 | 0.581 | >6.02 | 0.15 | 0.482 |
| 76 | 0.305 | 4.22 | 0.636 | 0.348 |
| 77 | 9.23 | >50 | >20 | n.d. |
| 78 | 0.307 | 6.41 | 1.907 | 0.553 |
| 79 | 7.28 | >50 | >20 | 7.35 |
| 80 | 0.089 | 0.244 | 0.063 | 0.059 |
| 81 | >25 | >50 | >20 | >50 |
| 82 | 4.99 | 3.9 | 1.213 | 4.2 |
| 83 | 15.45 | 6.81 | 7.04 | >50 |
| 84 | >25 | 9.91 | 8.62 | n.d. |
| 85 | >25 | 15.18 | 10.79 | 32.73 |
| 86 | >25 | >50 | >20 | 0.924 |
| 87 | >25 | >43.74 | >20 | >50 |
| 88 | 5.37 | 19.69 | 1.17 | 1.177 |
| 89 | >25 | >50 | n.d. | n.d. |
| 90 | 0.894 | 7.58 | 1.463 | 0.61 |
| 91 | 4.61 | 24.14 | n.d. | n.d. |
| 92 | >25 | >28.87 | >20 | n.d. |
| 93 | >25 | >50 | n.d. | n.d. |
| 94 | >25 | >50 | n.d. | n.d. |
| 95 | 0.069 | 0.188 | n.d. | n.d. |
| 96 | >15.77 | 42.24 | n.d. | 1.479 |
| 97 | 12.02 | 18.24 | 10.54 | 13.25 |
| 98 | 0.645 | 3.34 | 0.549 | 0.503 |
| 99 | 2.979 | 9.92 | n.d. | n.d. |
| 100 | >25 | >50 | n.d. | n.d. |
| 101 | 0.636 | 2.516 | 9.95 | 3.49 |
| 102 | 0.54 | 1.708 | 0.464 | 0.492 |
| 103 | 2.147 | 9.86 | 10.78 | 5.62 |
| 104 | 2.39 | >50 | 1.344 | 1.214 |
| 105 | 2.173 | >50 | 1.461 | 1.484 |
| 106 | 2.738 | 24.84 | 3.45 | 2.199 |
| 107 | 0.084 | 0.298 | n.d. | n.d. |
| 108 | 0.771 | 2.406 | 1.193 | n.d. |
| 109 | >25 | 28.22 | >20 | >50 |
| 110 | >25 | 8.1 | 9.59 | 27.7 |
| 111 | >25 | >50 | >20 | n.d. |
| 112 | 2.095 | 16.6 | 1.269 | 1.089 |
| 113 | 0.257 | 0.502 | 2.986 | 0.452 |
| 114 | 0.143 | 0.266 | 1.069 | 0.105 |
| 115 | 0.073 | 0.093 | 0.41 | 0.054 |
| 116 | 0.096 | 0.484 | 3.42 | 0.153 |
| 117 | 0.125 | >50 | >20 | 0.166 |
| 118 | 0.606 | 2.054 | 3.38 | 0.442 |
| 119 | 0.056 | 0.289 | 0.078 | 0.086 |
| 120 | 0.3 | 0.975 | 0.767 | 0.419 |
| 121 | 23.09 | >50 | >20 | 3.95 |
| 122 | 0.08 | n.d. | n.d. | n.d. |
| 123 | 0.423 | >50 | >20 | 0.414 |
| 124 | 0.049 | 5.64 | 0.046 | 0.023 |
| 125 | >21.9 | >50 | >20 | 7.94 |
| 126 | 0.434 | 4.05 | 4.86 | 0.126 |
| 127 | 0.561 | 2.12 | 1.983 | 0.074 |
| 128 | 1.748 | >50 | >20 | 0.171 |
| 129 | 0.435 | 1.265 | 1.336 | 0.123 |
| 130 | 13.89 | 26.63 | 7.12 | 4.66 |
| 131 | 0.317 | 1.142 | 1.051 | 0.06 |
| 132 | 2.866 | >50 | >13.78 | 1.547 |
| 133 | 0.097 | 0.607 | 1.842 | 0.019 |
| 134 | 0.267 | 8.36 | 5.2 | 0.073 |
| 135 | 0.255 | 3.126 | 3.79 | 0.106 |
| 136 | 0.392 | 1.416 | >20 | 0.157 |
| 137 | 2.147 | 24.76 | 8.01 | 0.406 |
| 138 | 0.549 | 3.88 | 1.95 | 0.127 |
| 139 | 0.45 | 6.82 | 3.74 | 0.128 |
| 140 | 1.266 | 8.38 | >17.61 | 0.378 |
| 141 | 0.862 | 8.28 | >20 | 1.361 |
| 142 | 0.063 | 0.506 | 0.179 | 0.121 |
| 143 | 0.478 | 1.396 | 7.91 | 0.159 |
| 144 | 0.555 | >50 | >20 | 0.164 |
| 145 | 0.223 | 0.464 | 1.73 | 0.045 |
| 146 | 1.046 | 7.18 | >20 | 0.236 |
| 147 | 0.074 | 0.067 | 0.065 | 0.019 |
| 148 | 0.051 | 0.222 | 0.09 | 0.061 |
| 149 | 0.241 | 0.617 | 1.876 | 0.516 |
| 150 | 0.532 | 43.79 | >20 | 0.506 |
| 151 | 0.136 | 0.429 | 0.59 | n.d. |
| 152 | 1.734 | 2.377 | 4.67 | 0.425 |
| 153 | 0.348 | 0.901 | 3.4 | 0.135 |
| 154 | 0.997 | 2.534 | 6.18 | 1.481 |
| 155 | 1.547 | 3.72 | 2.406 | 0.796 |
| 156 | 3.88 | >50 | >20 | 10.63 |
| 157 | >25 | >50 | >20 | 31.28 |
| 158 | 1.916 | >50 | >20 | 4.1 |
| 159 | 2.89 | 10.13 | 9.95 | 1.469 |
| 160 | >25 | >50 | >20 | 28.7 |
| 161 | 0.206 | 0.655 | 2.537 | 0.17 |
| 162 | 0.065 | 0.54 | 0.127 | 0.052 |
| 163 | 0.83 | 4.45 | 10.91 | 0.399 |
| 164 | 0.28 | 0.913 | 1.991 | 0.196 |
| 165 | >25 | >50 | 5.18 | 1.446 |
| 166 | 2.285 | >46.29 | >20 | 1.01 |
| 167 | 10.78 | >50 | >20 | 0.904 |
| 168 | 20.31 | 24.42 | >18.36 | 4.37 |
| 169 | 0.7 | 2.419 | 3.87 | 0.523 |
| 170 | 0.428 | 0.887 | 1.157 | 0.518 |
| 171 | 1.54 | >50 | >20 | 1.409 |
| 172 | 0.281 | 3.78 | 1.366 | 0.349 |
| 173 | 0.355 | 0.578 | 0.405 | 0.148 |
| 174 | n.d. | n.d. | n.d. | 0.483 |
| 175 | >25 | 12.64 | >20 | 4.48 |
| 176 | >25 | >50 | >20 | >50 |
| 177 | 0.31 | 0.911 | 3.47 | 0.161 |
| 178 | 6.71 | >42.54 | >20 | 2.973 |
| 179 | >25 | >45.85 | >20 | 6.96 |
| 180 | >25 | >50 | >20 | 4.38 |
| 181 | >25 | 33.14 | >20 | 12.7 |
| 182 | >25 | >50 | >20 | 10.76 |
| 183 | 3.63 | 11.48 | >20 | 2.096 |
| 184 | 0.146 | 0.333 | 0.565 | 0.175 |
| 185 | 0.462 | 1.139 | 1.073 | 0.954 |
| 186 | 15.84 | 6.22 | 10.23 | 0.59 |
| 187 | 0.031 | >50 | 0.142 | n.d. |
| 188 | 0.226 | >5.6 | 1.233 | 0.185 |
| 189 | 9.07 | 15.48 | 10.68 | 4.27 |
| 190 | 0.775 | 8.48 | 12.25 | 0.244 |
| 191 | 0.028 | 0.143 | 0.033 | n.d. |
| 192 | 3.24 | >25.12 | >20 | 1.135 |
| 193 | 0.339 | 0.717 | 1.09 | 0.201 |
| 194 | >25 | >50 | >20 | 29.84 |
| 195 | 5.64 | 5.33 | 2.04 | 0.463 |
| 196 | 6.52 | n.d. | n.d. | 1.865 |
| 197 | 2.491 | n.d. | n.d. | 0.491 |
| 198 | 0.007 | 0.016 | 0.016 | 0.018 |
| 199 | 0.878 | 6.44 | 1.057 | 1.115 |
| 200 | 0.219 | 0.631 | 0.147 | 0.164 |
| 201 | 0.058 | 0.208 | 0.126 | 0.46 |
| 202 | 0.229 | 2.719 | >20 | 0.249 |
| 203 | 0.97 | 13.81 | 10.59 | 1.156 |
| 204 | 0.941 | 19.19 | 4.1 | 0.676 |
| 205 | 0.3 | 1.243 | 0.413 | 0.352 |
| 206 | 3.19 | >50 | >20 | >50 |
| 207 | 7.14 | >40.28 | n.d. | n.d. |
| 208 | 0.134 | 0.445 | 0.1 | 0.108 |
| 209 | 0.087 | 0.317 | 0.13 | 0.169 |
| 210 | 0.047 | 0.085 | 0.031 | 0.034 |
| 211 | 0.021 | 0.055 | 0.016 | 0.029 |
| 212 | 1.121 | 4.15 | 1.3 | 1.013 |
| 213 | n.d. | n.d. | n.d. | 0.443 |
| 214 | 0.09 | 0.785 | 0.154 | 0.04 |
| 215 | 0.533 | 1.324 | n.d. | 0.181 |
| 216 | 4.43 | >50 | 11.54 | 3.128 |
| 217 | >25 | >50 | 7.06 | >50 |
| 218 | >25 | 31.69 | >20 | n.d. |
| 219 | 0.752 | 2.151 | n.d. | n.d. |
| 220 | 0.606 | 4.97 | 1.372 | n.d. |
| 221 | 0.249 | 1.058 | n.d. | 0.177 |
| 222 | 0.109 | 0.424 | 0.419 | 0.067 |
| 223 | 2.424 | 36.7 | >20 | 4.69 |
| 224 | 1.414 | >50 | >20 | 0.757 |
| 225 | 1.612 | 8.25 | 1.198 | 0.468 |
| 226 | 0.042 | 0.384 | n.d. | 0.037 |
| 227 | 3.74 | >50 | 4.91 | 4.37 |
| 228 | >25 | 19.48 | 9.99 | 34.54 |
| 229 | 0.884 | 2.82 | 10.99 | 0.776 |
| 230 | 2.732 | 11.69 | 1.311 | 0.323 |
| 231 | 0.307 | 1.718 | 0.42 | 0.313 |
| 232 | 0.381 | 1.648 | 0.384 | 0.203 |
| 233 | 0.29 | 2.051 | 0.754 | 0.326 |
| 234 | 0.219 | 0.741 | 0.155 | 0.151 |
| 235 | 0.215 | 0.821 | 0.433 | 0.165 |
| 236 | 0.095 | 0.434 | 0.098 | 0.036 |
| 237 | 0.217 | 0.877 | 0.229 | 0.122 |
| 238 | 0.208 | 0.788 | 0.233 | 0.117 |
| 239 | 0.515 | 2.2 | 0.372 | 0.35 |
| 240 | 1.176 | >50 | 0.602 | 0.439 |
| 241 | 0.303 | 2.953 | n.d. | n.d. |
| 242 | 0.687 | 2.201 | 0.198 | 0.477 |
| 243 | 0.78 | 2.613 | 0.15 | 0.369 |
| 244 | 0.152 | 0.345 | 0.055 | 0.065 |
| 245 | 1.693 | 2.031 | 1.127 | 1.398 |
| 246 | 0.562 | 1.19 | 0.622 | 0.085 |
| 247 | 0.74 | 3.25 | 0.575 | 0.367 |
| 248 | 0.191 | 0.411 | >20 | 0.169 |
| 249 | 3.37 | 5.55 | 1.335 | 4.07 |
| 250 | 0.498 | 0.839 | 0.631 | 0.112 |
| 251 | 0.035 | 0.085 | 0.061 | 0.045 |
| 252 | >25 | >50 | >20 | 33.02 |
| 253 | n.d. | n.d. | n.d. | n.d. |
| 254 | 6.9 | >18.32 | n.d. | n.d. |
| 255 | 0.364 | 0.502 | 0.329 | 0.122 |
| 256 | >25 | >50 | >20 | 31.59 |
| 257 | 9.22 | >33.77 | 4.05 | 4.65 |
| 258 | 20.17 | >42.45 | >20 | 13.14 |
| 259 | >22.14 | 23.73 | 10.9 | 34.64 |
| 260 | >25 | 3.83 | 4.55 | >50 |
| 261 | 4.32 | 7.03 | 1.902 | 1.219 |
| 262 | >25 | >50 | >20 | n.d. |
| 263 | 1.74 | 3.53 | 1.316 | 1.103 |
| 264 | 4.75 | >14 | 4.72 | 4.31 |
| 265 | 0.978 | 4.26 | 3.93 | 1.554 |
| 266 | 1.257 | 3.37 | 1.163 | 1.454 |
| 267 | 0.485 | 1.803 | 1.923 | 0.116 |
| 268 | >25 | 14.5 | 10.6 | n.d. |
| 269 | 9.59 | 15.1 | 4.16 | 3.57 |
| 270 | 2.9 | 2.203 | 0.701 | 4.11 |
| 271 | >25 | >50 | n.d. | n.d. |
| 272 | 1.687 | 3.37 | 1.29 | n.d. |
| 273 | >25 | >50 | >20 | n.d. |
| 274 | 0.862 | 1.904 | n.d. | n.d. |
| 275 | >25 | 17.14 | 11.71 | 34.66 |
| 276 | >25 | >50 | >20 | 8.72 |
| 277 | 6.88 | 10.7 | 3.9 | n.d. |
| 278 | 0.177 | 1.083 | 1.088 | n.d. |
| 279 | 2.441 | 9.59 | >17.48 | n.d. |
| 280 | >25 | >42.09 | >20 | n.d. |
| 281 | 8.7 | >37.1 | >20 | n.d. |
| 282 | >25 | 10.06 | 7.35 | n.d. |
| 283 | 0.244 | 0.724 | 1.322 | n.d. |
| 284 | >25 | 17.04 | 12.97 | n.d. |
| 285 | 11.69 | 16.14 | 5.02 | n.d. |
| 286 | >19.68 | >50 | >20 | 29.71 |
| 287 | 0.12 | 0.402 | 1.431 | 0.077 |
| 288 | 7.69 | >50 | n.d. | 33.78 |
| 289 | 0.141 | 0.321 | n.d. | n.d. |
| 290 | 3.145 | >45.68 | n.d. | 12.65 |
| 291 | 0.39 | 0.968 | n.d. | 0.206 |
| 292 | >25 | >43.53 | n.d. | n.d. |
| 293 | >25 | >38.23 | >16.51 | n.d. |
| 294 | >25 | >44.94 | >20 | n.d. |
| 295 | >25 | >50 | n.d. | n.d. |
| 296 | 0.004 | 0.013 | 0.014 | 0.012 |
| 297 | 0.052 | 0.223 | 0.037 | 0.051 |
| 298 | 0.781 | 2.234 | 0.525 | 0.355 |
| 299 | 0.092 | 0.178 | 0.045 | 0.034 |
| 300 | 0.09 | 0.397 | 0.148 | 0.155 |
| 301 | 0.727 | >32.35 | 0.444 | 0.307 |
| 302 | 0.305 | 0.587 | 0.134 | n.d. |
| 303 | >20.29 | >50 | 17.74 | n.d. |
| 304 | 0.539 | 10.54 | 2.372 | 1.421 |
| 305 | 0.112 | 0.269 | 0.499 | 0.163 |
| 306 | 0.315 | 0.835 | 0.673 | 0.36 |
| 307 | 0.249 | 0.428 | 2.535 | 0.356 |
| 308 | 0.211 | 0.338 | 0.329 | 0.131 |
| 309 | 0.109 | 0.204 | 0.275 | 0.132 |
| 310 | 0.243 | 0.564 | 1.237 | 0.293 |
| 311 | 0.162 | 11.97 | 5.58 | 1.669 |
| 312 | 0.12 | 0.139 | 0.441 | 0.054 |
| 313 | 0.761 | >50 | 13.27 | 0.502 |
| 314 | 0.046 | 0.095 | 0.015 | 0.051 |
| 315 | 2.524 | 19.06 | >19.78 | 4.41 |
| 316 | >25 | >50 | n.d. | >50 |
| 317 | 0.014 | 0.038 | 0.012 | 0.012 |
| 318 | 0.031 | 0.132 | 0.146 | 0.019 |
| 319 | 0.033 | 0.102 | 0.02 | 0.02 |
| 320 | 0.058 | 0.18 | 0.186 | 0.035 |
| 321 | 0.008 | 0.015 | 0.005 | n.d. |
| 322 | 0.043 | 0.157 | 0.076 | 0.02 |
| 323 | 0.097 | 0.278 | 0.046 | 0.047 |
| 324 | 0.288 | 1.566 | 0.253 | 0.047 |
| 325 | 0.154 | 0.722 | n.d. | n.d. |
| 326 | 0.158 | 0.496 | 0.123 | n.d. |
| 327 | 0.167 | 0.459 | 0.05 | 0.102 |
| 328 | 0.387 | 1.5 | 0.302 | 0.167 |
| 329 | 0.026 | 0.057 | 0.007 | 0.055 |
| 330 | 0.028 | 0.114 | 0.104 | 0.036 |
| 331 | 0.396 | 0.36 | 0.053 | 0.116 |
| 332 | 0.166 | 0.592 | 0.134 | 0.124 |
| 333 | 0.088 | 0.087 | 0.016 | n.d. |
| 334 | 0.052 | 0.085 | 0.064 | n.d. |
| 335 | 0.178 | 0.521 | 0.407 | 0.114 |
| 336 | 0.074 | 0.168 | 0.045 | 0.086 |
| 337 | 0.072 | 0.195 | 0.068 | 0.024 |
| 338 | 0.047 | 0.101 | 0.018 | 0.04 |
| 339 | 0.085 | 0.272 | n.d. | 0.08 |
| 340 | 0.707 | 2.115 | n.d. | n.d. |
| 341 | 2.599 | 9.43 | n.d. | n.d. |
| 342 | 2.14 | 14.56 | 9.36 | 0.364 |
| 343 | 0.106 | 0.308 | 0.074 | n.d. |
| 344 | 0.073 | 0.158 | 0.051 | n.d. |
| 345 | 0.251 | 0.955 | 0.408 | 0.527 |
| 346 | 0.355 | 0.49 | 0.159 | n.d. |
| 347 | 0.154 | 0.151 | 0.099 | n.d. |
| 348 | 0.341 | 1.033 | 0.041 | 0.165 |
| 349 | 0.074 | 0.672 | 0.18 | 0.069 |
| 350 | 0.016 | 0.128 | n.d. | 0.053 |
| 351 | 0.071 | 0.13 | 0.095 | n.d. |
| 352 | 0.215 | 0.061 | 0.018 | n.d. |
| 353 | 0.051 | 0.112 | 0.053 | n.d. |
| 354 | 0.026 | 0.103 | 0.008 | 0.004 |
| 355 | 0.097 | 0.521 | 1.135 | 0.086 |
| 356 | 0.088 | 0.332 | 0.016 | 0.018 |
| 357 | 0.088 | 0.194 | 0.015 | n.d. |
| 358 | 0.03 | 0.148 | 0.012 | n.d. |
| 359 | 0.45 | 4.86 | 0.405 | 1.498 |
| 360 | 3.37 | 0.45 | 0.252 | 0.116 |
| 361 | 7.93 | 0.993 | 1.04 | n.d. |
| 362 | 0.403 | >34.42 | >20 | 0.345 |
| 363 | 0.077 | 0.136 | 0.045 | 0.058 |
| 364 | 0.279 | 0.155 | 0.22 | 0.178 |
| 365 | 0.102 | 0.2 | n.d. | 0.059 |
| 366 | 0.849 | 0.339 | 0.443 | 0.338 |
| 367 | >25 | >50 | n.d. | n.d. |
| 368 | 0.463 | 0.905 | 0.135 | 0.498 |
| 369 | 0.282 | 0.294 | 0.988 | 0.154 |
| 370 | 5.47 | 3.61 | 7.37 | 0.386 |
| 371 | 1.652 | 2.302 | 0.465 | 1.47 |
| 372 | 0.221 | 0.242 | 0.095 | 0.08 |
| 373 | 0.115 | 0.134 | 0.084 | 0.044 |
| 374 | 0.04 | 0.043 | 0.041 | 0.425 |
| 375 | 4.3 | 0.499 | 0.303 | 0.135 |
| 376 | 0.085 | 0.256 | 0.096 | 0.158 |
| 377 | 0.075 | 0.144 | 0.137 | 0.042 |
| 378 | 0.543 | 0.134 | 0.047 | 0.038 |
| 379 | 0.528 | 1.679 | 0.683 | 1.434 |
| 380 | 24.47 | 33.86 | 14.05 | 30.71 |
| 381 | 0.392 | 1.441 | 0.445 | 0.482 |
| 382 | 3.88 | 10.66 | 1.39 | 2.604 |
| 383 | 0.146 | 0.521 | 0.137 | 0.331 |
| 384 | 0.095 | 0.616 | 0.115 | 0.143 |
| 385 | 0.268 | >36.7 | 1.162 | 0.369 |
| 386 | 1.496 | >50 | >20 | 1.554 |
| 387 | 0.086 | 0.286 | 0.149 | n.d. |
| 388 | 1.008 | 2.953 | 3.53 | 0.415 |
| 389 | 0.097 | 0.33 | 0.254 | 0.158 |
| 390 | 0.053 | 0.174 | 0.075 | 0.054 |
| 391 | 0.056 | 0.129 | 0.016 | 0.053 |
| 392 | 0.103 | 0.152 | 0.048 | 0.055 |
| 393 | 0.147 | 0.183 | 0.149 | 0.11 |
| 394 | 0.08 | 0.107 | >20 | n.d. |
| 395 | 0.109 | 0.187 | 0.153 | 0.045 |
| 396 | 0.055 | 0.088 | 0.017 | 0.042 |
| 397 | 0.337 | 0.448 | 0.2 | 0.068 |
| 398 | 0.186 | 0.424 | 0.572 | 0.317 |
| 399 | 0.274 | 0.646 | 0.08 | 0.103 |
| 400 | 0.045 | 0.203 | 0.199 | n.d. |
| 401 | 0.094 | 0.263 | 0.445 | 0.034 |
| 402 | 0.069 | 0.285 | 0.251 | 0.011 |
| 403 | 0.063 | 0.181 | n.d. | 0.055 |
| 404 | 0.011 | 0.034 | 0.017 | n.d. |
| 405 | 0.094 | 0.361 | 0.271 | 0.013 |
| 406 | 0.266 | 1.516 | 0.436 | n.d. |
| 407 | 0.009 | 0.014 | 0.014 | 0.004 |
| 408 | 0.078 | 0.246 | 0.237 | 0.018 |
| 409 | 0.019 | 0.061 | 0.005 | 0.004 |
| 410 | 0.022 | 0.065 | n.d. | 0.017 |
| 411 | 0.083 | 0.154 | 0.046 | 0.022 |
| 412 | 0.046 | 0.134 | 0.048 | 0.04 |
| 413 | 0.026 | 0.054 | 0.014 | 0.039 |
| 414 | 0.217 | 1.163 | 0.115 | 0.131 |
| 415 | 0.028 | 0.114 | 0.044 | 0.012 |
| 416 | 0.03 | 0.093 | 0.047 | 0.041 |
| 417 | 0.25 | 1.3 | 0.135 | 0.145 |
| 418 | 0.029 | 0.071 | 0.015 | 0.008 |
| 419 | 0.077 | 0.182 | 0.114 | n.d. |
| 420 | 0.83 | 1.087 | 0.307 | 0.52 |
| 421 | 0.127 | 0.337 | 0.17 | n.d. |
| 422 | 0.126 | 0.305 | 0.193 | 0.033 |
| 423 | 0.09 | 0.217 | 0.048 | 0.035 |
| 424 | 0.056 | 0.164 | 0.118 | 0.054 |
| 425 | 0.14 | 0.198 | 0.05 | 0.097 |
| 426 | 0.016 | 0.182 | 0.124 | 0.378 |
| 427 | 0.237 | 0.438 | 1.333 | 0.163 |
| 428 | 0.1 | 0.291 | 0.451 | 0.161 |
| 429 | n.d. | n.d. | n.d. | n.d. |
| 430 | 0.031 | 0.059 | 0.01 | 0.052 |
| 431 | 0.079 | 0.845 | 0.046 | 0.149 |
| 432 | 0.012 | 0.033 | 0.021 | 0.018 |
| 433 | 1.813 | >50 | >20 | 0.494 |
| 434 | 0.107 | 0.924 | 0.042 | 0.16 |
| 435 | 0.019 | 0.049 | 0.016 | 0.042 |
| 436 | 0.077 | 1.715 | 0.05 | 0.072 |
| 437 | 0.08 | 1.248 | 0.682 | 0.403 |
| 438 | 0.141 | 0.282 | 0.146 | 0.109 |
| 439 | 0.108 | 0.754 | 0.089 | 0.121 |
| 440 | 0.119 | 0.2 | 0.238 | 0.315 |
| 441 | 0.097 | 0.912 | 0.141 | 0.309 |
| 442 | 0.087 | 0.208 | 0.048 | 0.055 |
| 443 | 0.081 | 0.146 | 0.103 | 0.034 |
| 444 | 0.086 | 0.183 | 0.05 | 0.025 |
| 445 | 0.012 | 0.688 | 0.014 | 0.031 |
| 446 | 0.033 | 0.146 | 0.122 | 0.161 |
| 447 | 0.036 | 1.376 | 0.02 | 0.103 |
| 448 | 0.108 | 5.15 | 0.798 | 0.157 |
| 449 | 0.028 | 0.054 | 0.022 | 0.056 |
| 450 | 4.62 | 2.993 | 0.397 | 0.499 |
| 451 | 6.47 | 1.934 | 1.949 | 0.968 |
| 452 | 0.019 | 0.061 | 0.045 | 0.097 |
| 453 | 0.017 | 0.023 | 0.015 | 0.021 |
| 454 | 0.009 | 0.024 | 0.014 | 0.018 |
| 454 | 0.035 | 0.085 | 0.061 | 0.045 |
| 455 | 0.225 | 0.681 | 0.215 | 0.134 |
| 456 | 0.042 | 0.218 | 0.04 | 0.054 |
| 457 | 0.162 | 13.16 | 0.32 | n.d. |
| 458 | 0.075 | 0.513 | 0.043 | 0.12 |
| 459 | 0.129 | 0.347 | 0.119 | n.d. |
| 460 | 0.241 | 0.208 | 0.182 | 0.164 |
| 461 | 0.234 | 0.185 | 0.232 | 0.055 |
| 462 | 0.174 | 0.403 | 0.151 | 0.129 |
| 463 | 0.571 | 0.507 | 0.163 | 0.238 |
| 464 | 0.47 | 0.672 | 0.148 | 0.109 |
| 465 | 0.329 | 0.289 | 0.146 | 0.153 |
| 466 | 0.158 | 0.207 | 0.183 | 0.044 |
| 467 | 0.147 | 0.266 | 0.129 | 0.166 |
| 468 | 0.087 | 0.23 | 0.146 | 0.015 |
| 469 | 0.143 | 0.23 | 0.146 | 0.055 |
| 470 | 0.252 | 0.689 | 0.148 | 0.159 |
| 471 | 0.601 | 0.637 | 0.147 | 0.159 |
| 472 | 0.21 | 0.737 | 0.297 | 0.058 |
| 473 | 1.028 | 1.639 | 0.38 | 0.043 |
| 474 | 0.292 | 1.108 | 0.478 | 0.203 |
| 475 | 1.31 | 5.98 | 9.16 | 0.217 |
| 476 | 0.153 | 0.358 | 0.152 | 0.042 |
| 477 | 0.751 | 0.425 | 0.386 | 0.055 |
| 478 | 4.72 | >16.38 | 5.52 | 0.533 |
| 479 | >22.76 | >50 | >20 | 41.94 |
| 480 | >22.31 | >50 | >20 | 8.42 |
| 481 | 0.209 | 0.43 | 0.151 | n.d. |
| 482 | 0.569 | 1.011 | 0.547 | 0.494 |
| 483 | 0.169 | 0.196 | 0.074 | 0.07 |
| 484 | 1.48 | 9.11 | 0.361 | 1.356 |
| 485 | >25 | 11.5 | 1.971 | >50 |
| 486 | 16.1 | >50 | 1.451 | n.d. |
| 487 | >25 | >50 | >16.59 | >50 |
| 488 | 0.032 | 0.132 | 0.036 | 0.058 |
| 489 | 1.772 | 7.79 | 6.71 | 0.152 |
| 490 | 0.134 | 0.383 | 0.157 | n.d. |
| 491 | 0.234 | 0.743 | 0.296 | 0.14 |
| 492 | 0.841 | 1.525 | 0.31 | 0.472 |
| 493 | 0.26 | 0.192 | 0.031 | 0.04 |
| 494 | 3.52 | 4.06 | 1.299 | 1.011 |
| 495 | 0.235 | 0.233 | 0.055 | 0.036 |
| 496 | 2.055 | 8.85 | 2.115 | n.d. |
| 497 | 0.641 | 1.282 | 0.112 | 0.496 |
| 498 | 0.849 | 28.01 | 1.345 | 0.478 |
| 499 | >25 | 8.26 | 9.42 | 4.67 |
| 500 | >25 | >17.55 | >20 | >50 |
| 501 | >25 | >50 | 10.46 | 3.14 |
| 502 | >25 | 16.02 | 10.97 | 6.73 |
| 503 | >25 | 26.64 | >20 | 36.72 |
| 504 | 2.973 | 5.51 | 10.74 | 0.976 |
| 505 | 1.17 | 3.48 | 5.0 | 0.845 |
| 506 | >25 | >25.95 | >18.23 | n.d. |
| 507 | 1.848 | 5.8 | 9.92 | 1.263 |
| 508 | 1.71 | 8.24 | 11.54 | 4.36 |
| 509 | >25 | >50 | >20 | 10.35 |
| 510 | 1.601 | 8.13 | 2.609 | 4.01 |
| 511 | 1.552 | 2.279 | 3.68 | 0.59 |
| 512 | 0.211 | 2.992 | n.d. | 0.076 |
| 513 | 0.958 | 1.579 | n.d. | 0.426 |
| 514 | 0.641 | 1.644 | n.d. | 0.084 |
| 515 | 7.37 | n.d. | n.d. | 13.53 |
| 516 | >25 | 21.02 | n.d. | >50 |
| 517 | 4.59 | >50 | n.d. | 1.428 |
| 518 | 0.785 | n.d. | n.d. | 0.386 |
| 519 | 0.244 | 1.623 | 3.26 | 0.366 |
| 520 | >25 | >50 | >20 | n.d. |
| 521 | 4.68 | 38.19 | 9.23 | n.d. |
| 522 | 19.28 | >50 | >20 | n.d. |
| 523 | >25 | 37.72 | n.d. | >50 |
| 524 | 3.51 | 9.54 | 12.65 | 1.001 |
| 525 | 5.35 | 21.09 | >20 | n.d. |
| 526 | >25 | >50 | >20 | >50 |
| 527 | >25 | 5.77 | >20 | >50 |
| 528 | >25 | 8.94 | >20 | 6.96 |
| 529 | 7.23 | >50 | n.d. | 3.26 |
| 530 | >13.12 | >50 | >20 | >50 |
| 531 | >25 | n.d. | n.d. | >50 |
| 532 | 13.94 | >50 | n.d. | 17.75 |
| 533 | n.d. | n.d. | n.d. | 1.539 |
| 534 | >23.67 | >50 | >20 | n.d. |
| 535 | >25 | >35.53 | >20 | 31.09 |
| 536 | 0.348 | 2.003 | 0.153 | n.d. |
| 537 | 0.124 | 0.286 | 0.103 | 0.164 |
| 538 | 2.364 | 1.859 | 0.447 | 0.613 |
| 539 | 0.975 | n.d. | 0.381 | n.d. |
| 540 | >25 | n.d. | 1.328 | 0.868 |
| 541 | >25 | n.d. | >20 | 9.92 |
| 542 | 0.45 | n.d. | 8.06 | 0.467 |
| 543 | 0.212 | 0.759 | 0.882 | 0.134 |
| 544 | 4.04 | 19.27 | 4.01 | >50 |
| 545 | 3.9 | 2.134 | 7.18 | 3.25 |
| 546 | 5.66 | 30.16 | 3.85 | 3.18 |
| 547 | 0.856 | 4.53 | 1.36 | 0.607 |
| 548 | 0.651 | 1.069 | 0.539 | 0.518 |
| 549 | 0.133 | 0.448 | 0.063 | 0.117 |
| 550 | 0.176 | 0.516 | 0.042 | 0.078 |
| 551 | 10.99 | 6.94 | 3.28 | 9.32 |
| 552 | 0.42 | 0.967 | 0.326 | 0.24 |
| 553 | 3.48 | 3.61 | 3.107 | 4.29 |
| 554 | 0.814 | 1.854 | 1.512 | 0.664 |
| 555 | 3.112 | 5.47 | 1.322 | 1.569 |
| 556 | 0.063 | 0.099 | 0.313 | 0.07 |
| 557 | >25 | >50 | >20 | n.d. |
| 558 | >25 | >50 | n.d. | 17.24 |
| 559 | >25 | >50 | >20 | n.d. |
| 560 | 9.84 | 29.7 | 12.14 | 5.65 |
| 561 | 0.74 | 2.136 | 0.139 | 0.286 |
| 562 | 0.16 | 0.245 | 0.046 | n.d. |
| 563 | 14.21 | 8.5 | 6.7 | >50 |
| 564 | 0.212 | 0.29 | 0.204 | 0.056 |
| 565 | 12.26 | 6.27 | 11.21 | n.d. |
| 566 | 1.137 | 12.03 | n.d. | 0.212 |
| 567 | >25 | >50 | n.d. | 12.09 |
| 568 | n.d. | n.d. | n.d. | 9.84 |
| 569 | >25 | n.d. | n.d. | >50 |
| 570 | 1.816 | 13.58 | 1.991 | 0.248 |
| 571 | >25 | >50 | >20 | n.d. |
| 572 | 1.941 | 2.363 | 0.394 | n.d. |
| 573 | 0.155 | 0.168 | 0.462 | 0.107 |
| 574 | 0.744 | 0.886 | 0.436 | 0.46 |
| 575 | 0.078 | n.d. | 0.052 | 0.022 |
| 576 | 0.526 | 0.807 | 0.05 | n.d. |
| 577 | 2.991 | >50 | 2.341 | 1.348 |
| 578 | n.d. | n.d. | n.d. | n.d. |
| 579 | >25 | >50 | n.d. | 36.18 |
| 580 | >25 | n.d. | n.d. | >50 |
| 581 | 0.169 | 0.249 | 0.11 | 0.101 |
| 582 | 0.585 | 0.928 | 0.466 | 0.476 |
| 583 | 0.869 | 1.787 | 0.377 | 0.139 |
| 584 | >25 | 2.267 | n.d. | >50 |
| 585 | n.d. | n.d. | n.d. | 0.414 |
| 586 | 0.552 | 4.0 | n.d. | 0.059 |
| 587 | 0.073 | 0.647 | n.d. | n.d. |
| 588 | 0.019 | 0.459 | n.d. | n.d. |
| 589 | 0.022 | 0.375 | n.d. | n.d. |
| 590 | 0.024 | 0.045 | n.d. | n.d. |
| 591 | 0.028 | >50 | n.d. | n.d. |
| 592 | 0.145 | 0.428 | n.d. | 0.053 |
| 593 | 0.255 | 0.16 | n.d. | 0.054 |
| 594 | >25 | >32.83 | >19.88 | >50 |
| 595 | >25 | >50 | 9.17 | n.d. |
| 596 | >21.24 | 0.075 | 3.74 | n.d. |
| 597 | 24.06 | 17.13 | 15.97 | 31.44 |
| 598 | 2.767 | 10.28 | 2.546 | 4.36 |
| 599 | 2.69 | 0.639 | 3.93 | 3.3 |
| 600 | 1.464 | >50 | 1.751 | 1.779 |
| 601 | >25 | 4.04 | >20 | >50 |
| 602 | >25 | >50 | 13.62 | >50 |
| 603 | 17.79 | 12.65 | 5.84 | 8.17 |
| 604 | 0.022 | 0.041 | 0.046 | 0.051 |
| 605 | 0.021 | 0.059 | 0.016 | 0.013 |
| 606 | 0.545 | 3.2 | 0.38 | 0.267 |
| 607 | 0.705 | 0.443 | 0.437 | 1.04 |
| 608 | 0.127 | 0.456 | 0.132 | 0.159 |
| 609 | 0.835 | 5.97 | >20 | 1.05 |
| 610 | 0.121 | 0.269 | 0.109 | 0.15 |
| 611 | 0.645 | 1.551 | 1.134 | 0.611 |
| 612 | 9.4 | 1.059 | 3.81 | 12.44 |
| 613 | 0.03 | >50 | n.d. | n.d. |
| 614 | 0.389 | >50 | n.d. | n.d. |
| 615 | 0.082 | n.d. | n.d. | n.d. |
| 616 | 0.602 | >50 | n.d. | n.d. |
| 617 | 3.44 | n.d. | n.d. | n.d. |
| 618 | 0.903 | 0.691 | n.d. | n.d. |
| 619 | 6.35 | 0.929 | n.d. | n.d. |

n.d. means not determined.

### EXAMPLES

The present invention is further exemplified, but not to be limited, by the following examples and intermediates that illustrate the preparation of compounds of the invention. It is understood that if there appears to be a discrepancy between the name and structure of a particular compound, the structure is to be considered correct as the compound names were generated from the structures.

Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are [symbol] one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

### Method for Analysis:

### Method 1:

Waters Acquity UPLC system:

Acquity Binary Gradient Manager with Degasser

Acquity Column Compartment set at 50° C.

Acquity Diode Array Detector

### Leap Technologies HTS Pal Autosampler

### Antek Chemiluminescent Nitrogen Detector (CLND)

### Waters ZQ2000 Mass Spectrometer

### HPLC Column: Thermo Syncronis C18 30×2.1 mm

Mobile Phase: (A)95% H2O/5% MeOH/IPA (75/25, v/v)+0.05% formic acid, (B)
MeOH/IPA (75/25, v/v)+0.035% formic acid
Gradient: 0.4 mL/minute, initial 2% B for 1.0 minutes, ramp to 95% B over 2.5 minutes, until 4.0 minutes, return to 2% B to at 4.25 minutes until end of run at 5.0.
MS Scan: 150 to 1000 amu in 1 second
Diode Array Detector: 190 nm-400 nm

### Method 2:

Waters Acquity UPLC system:

Acquity Binary Gradient Manager with Degasser

Acquity Column Compartment set at 50° C.

Acquity Diode Array Detector

### Leap Technologies HTS Pal Autosampler

### Waters ZQ2000 or SQDII Mass Spectrometer

### HPLC Column: Waters Acquity C18 1.7 um 2.1×30 mm

### Mobile Phase: (A)H2O+0.05% TFA and (B)Acetonitrile+0.05% TFA

Gradient: 1 mL/minute, initial 5% B for 0.1 minutes, ramp to 95% B over 1.5 minutes, hold until 1.6 minutes then to 100% B at 1.7 and return to 5% B to at 1.9 minutes until end of run at 2.25.
MS Scan: 180 to 800 amu in 0.4 seconds
Diode Array Detector: 214 nm-400 nm

### Method 3:

Agilent G1379A Degasser

Agilent G1312A Binary Pump

Agilent G1315B Diode Array Detector

Leap Technologies HTS Pal Autosampler

Sedex 75 Evaporative Light Scattering Detector

Waters ZQ2000 Mass Spectrometer

HPLC Column: Thermo Accucore aQ C18 2.6 um 30×2.1 mm

Mobile Phase: (A)H₂O+0.05% TFA and (B)Acetonitrile+0.05% TFA

Gradient: 1 mL/minute, initial 5% B for 0.1 minutes, ramp to 90% B over 2.5 minutes, then to 100% B at 2.61 and hold until 3.10 minutes, return to 5% B to at 3.15 minutes until end of run at 3.25. The column is re-equilibrated in the ˜30 seconds between injections.

MS Scan: 180 to 800 amu in 0.4 seconds

Diode Array Detector: 214 nm-400 nm

### Method 4:

Waters Acquity UPLC system:

Acquity Binary Gradient Manager with Degasser

Acquity Diode Array Detector

### Leap Technologies HTS Pal Autosampler

### Waters Waters SQD Mass Spectrometer

### HPLC Column: Waters Acquity C18 1.7 um 2.1×30 mm

### Mobile Phase: (A)H2O+0.05% TFA and (B)Acetonitrile+0.05% TFA

Gradient: 1 mL/minute, initial 5% B for 0.1 minutes, ramp to 95% B over 1.5 minutes, hold until 1.6 minutes then to 100% B at 1.7 and return to 5% B to at 1.9 minutes until end of run at 2.25.
MS Scan: 180 to 800 amu in 0.4 seconds
Diode Array Detector: 214 nm-400 nm

### Method 5:

### Column: 21×250 mm ChiralPak AS-H

### Solvents: (A) Hexane (75%); (B) 1:1 (Ethanol:Methanol) 25%

Flow rate: 1 mL/min

### Phase: 3 um 4.6×50 mm ChiralPak AS (HPLC)

Prep conditions: 20 mL/min, 72/14/14 Hexane/EtOH/MeOH
Run time 17 minutes

### Synthesis of Intermediates

### Example 1

### Synthesis of 3-(6-bromoimidazo[1,2-a]pyrimidin-2-yl)-4-chloroaniline (I-4)

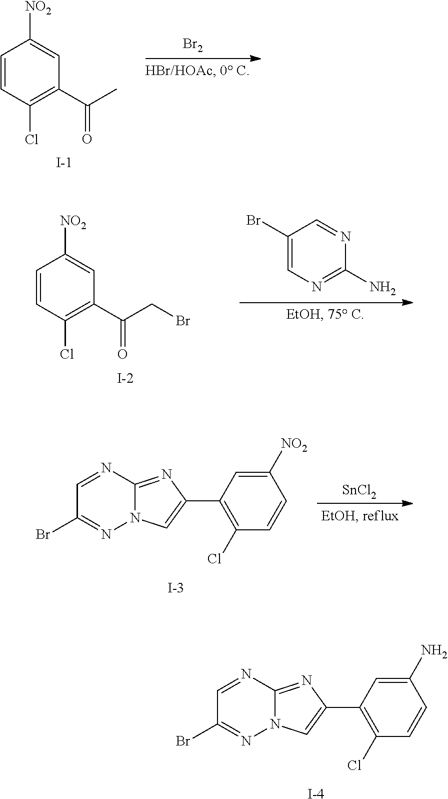

### Synthesis of I-2

To a solution of 1-(2-chloro-5-nitrophenyl)ethanone I-1 (30 g, 150.75 mmol, 1 eq.) in AcOH (250 mL) was added Br₂ (18.57 g, 116 mmol, 0.77 eq.) in AcOH (50 mL) dropwise and the resulting mixture was stirred for 12 h at 70° C. The reaction mixture was concentrated under vacuum. The residue was diluted with 200 mL of water and extracted with Ethyl Acetate (2×200 mL). The organic layer was washed with water and brine, dried over Na₂SO₄ and concentrated under vacuum to give 30 g (71%) of 2-bromo-1-(2-chloro-5-nitrophenyl)ethanone I-2 as a brown oil.

### Synthesis of I-3

A solution of 2-bromo-1-(2-chloro-5-nitrophenyl)ethan-1-one I-2 (30 g, 108 mmol, 1 eq.) and 5-bromopyrimidin-2-amine (15 g, 88 mmol, 0.81 eq.) in ethanol (300 mL) was stirred for 12 hours at 75° C. and then concentrated under vacuum. The residue was diluted with 200 mL of water and extracted with Ethyl Acetate (3×100 mL). The combined organic layers were washed with brine, dried over Na₂SO₄ and concentrated under vacuum to give 15 g (39%) of 6-bromo-2-(2-chloro-5-nitrophenyl)imidazo[1,2-a]pyrimidine I-3 as a brown solid.

### Synthesis of I-4

To a suspension of 6-bromo-2-(2-chloro-5-nitrophenyl)imidazo[1,2-a]pyrimidine I-3 (10 g, 28.28 mmol, 1 eq.) in ethanol (100 mL) was added SnCl₂.2H₂O (12.7 g, 56 mmol, 2 eq.) and the solution was stirred for 12 hours at 75° C. The solvent was removed under vacuum and the residue was diluted with water. The pH value of the mixture was adjusted to 8-9 with a saturated sodium bicarbonate solution. The solids were collected by filtration and applied onto a silica gel column with Ethyl Acetate/Petroleum Ether (2/1) to give 2.6 g (28%) of 3-[6-bromoimidazo[1,2-a]pyrimidin-2-yl]-4-chloroaniline I-4 as a light yellow solid.

¹H NMR (400 MHz, DMSO-d₆) δ 9.37 (d, J=2.4 Hz, 1H), 8.63 (d, J=2.4 Hz, 1H), 8.47 (s, 1H), 7.56 (d, J=2.8 Hz, 1H), 7.18 (d, J=8.4 Hz, 1H), 6.59 (dd, J=8.4, 2.8 Hz, 1H), 5.44 (s, 2H). MS m/z=325 (M+1).

### Example 2

### Synthesis of isopropyl (2-(3-aminophenyl)imidazo[1,2-a]pyrimidin-6-yl)carbamate (I-8)

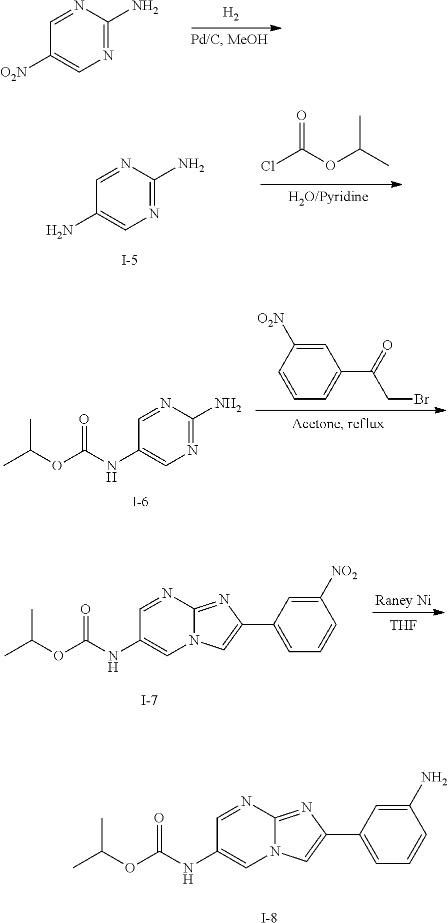

### Synthesis of I-5

Into a 1 L round-bottom flask, was placed 5-nitropyrimidin-2-amine (30 g, 214.13 mmol, 1 eq.), methanol (400 mL), and Palladium on carbon (12 g). The resulting solution was stirred overnight at room temperature under an hydrogen atmosphere. The solids were filtered out. The resulting mixture was concentrated under vacuum giving 20 g (85%) of pyrimidine-2,5-diamine I-5 as a light brown solid.

### Synthesis of I-6

To a 1 L round-bottom flask, containing pyrimidine-2,5-diamine I-5 (20 g, 181.62 mmol, 1 eq.), water (500 mL), and isopropyl chloroformate (33.3 g, 271.73 mmol, 1.50 eq.) was added pyridine (71.8 g, 907.71 mmol, 5.00 eq.) dropwise while stirring. The resulting solution was stirred for 5 h at room temperature. The mixture was then extracted with 5×500 mL of Ethyl Acetate and the organic layers combined and concentrated under vacuum. The residue was applied onto a silica gel column with Ethyl Acetate/petroleum ether (2:1). This resulted in 26 g (73%) of propan-2-yl N-(2-aminopyrimidin-5-yl)carbamate I-6 as a yellow solid.

### Synthesis of I-7

Into a 500 mL round-bottom flask, was placed propan-2-yl N-(2-aminopyrimidin-5-yl)carbamate I-6 (6 g, 30.58 mmol, 1.00 eq.), acetone (300 mL) and 2-bromo-1-(3-nitrophenyl)ethan-1-one (8.2 g, 33.60 mmol, 1.10 eq.). The resulting solution was stirred overnight at 70° C. The reaction mixture was cooled and the solids were collected by filtration resulting in 8 g (77%) of propan-2-yl N-[2-(3-nitrophenyl)imidazo[1,2-a]pyrimidin-6-yl]carbamate I-7 as a yellow solid.

### Synthesis of I-8

Into a 1-L round-bottom flask, was placed tetrahydrofuran (300 mL), Raney Ni (10 g) and propan-2-yl N-[2-(3-nitrophenyl)imidazo[1,2-a]pyrimidin-6-yl]carbamate I-7 (7.5 g, 21.97 mmol, 1 eq.). The resulting solution was stirred for 5 h at room temperature under an hydrogen atmosphere. The solids were filtered out, and washed with 5×150 mL MeOH. The resulting mixture was concentrated under vacuum. This resulted in 6.0 g (88%) of propan-2-yl N-[2-(3-aminophenyl)imidazo[1,2-a]pyrimidin-6-yl]carbamate I-8 as a yellow solid. ¹H NMR (300 MHz, DMSO-d₆) δ 10.10 (s, 1H), 9.30 (s, 1H), 8.54 (s, 1H), 8.41 (s, 1H), 7.23-7.37 (m, 3H), 6.79-6.85 (m, 1H), 4.92-4.98 (m, 1H), 3.17 (s, 2H), 1.13-1.31 (m, 6H). MS m/z=312 (M+1).

The reduction can be also carried out using tin chloride following the protocol described in the synthesis of 1-4.

### Example 3

### Synthesis of isopropyl (2-(5-amino-2-chlorophenyl)imidazo[1,2-a]pyrimidin-6-yl)carbamate (I-9)

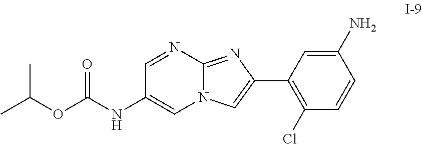 [SMILES: CC(C)OC(=O)Nc1cnc2nc(-c3cc(N)ccc3Cl)cn2c1]

Intermediate I-9 was synthesized using the synthetic protocol described for I-8, 2-bromo-1-(2-chloro-5-nitrophenyl)ethan-1-one was used in lieu of 2-bromo-1-(3-nitrophenyl)ethan-1-one for the cyclization step as described in the synthesis of 1-7. ¹H NMR (300 MHz, CD₃OD) δ 9.30 (s, 1H), 8.51 (d, J=2.1 Hz, 1H), 8.37 (s, 1H), 7.37 (d, J=1.8 Hz, 1H), 7.23 (d, J=6.6 Hz, 1H), 6.72-6.74 (m, 1H), 5.01-5.07 (m, 1H), 1.24-1.36 (m, 6H). MS m/z=346 (M+1).

### Example 4

### Synthesis of isopropyl (2-(5-amino-2-fluorophenyl)imidazo[1,2-a]pyrimidin-6-yl)carbamate (I-10)

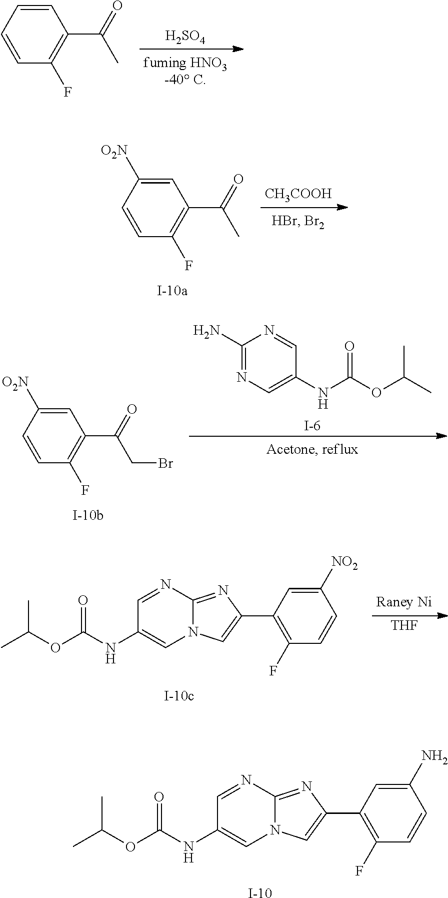

### Synthesis of I-10a

A 3000 mL three necked flask equipped with a mechanic stirrer was charged with concentrated H₂SO₄ (720 mL) and cooled to −40° C. 1-(2-Fluorophenyl)ethanone (180 g, 1.3 mol) was added, followed by addition of a mixture of fuming HNO₃ (106.2 mL) in concentrated H₂SO₄ (260 mL) dropwise over 45 minutes. This mixture was stirred at this temperature for 15 minutes, poured into ice (8 kg), and extracted with Ethyl Acetate (2000 mL×2). The combined Ethyl Acetate layer was washed with sat NaHCO₃ solution (800 mL×3), brine (800 mL), dried with anhydrous sodium sulfate, and concentrated under vacuo. The residue was crystallized with petroleum ether to give compound I-10a (200 g, yield: 84%) as a yellow solid. ¹H NMR (400 MHz, CDCl₃) δ 7.34 (t, J=9.29 Hz, 1H), 8.33-8.48 (m, 1H), 8.78 (dd, J=6.15, 2.89 Hz, 1H).

### Synthesis of I-10b

To a solution of compound I-10a (126 g, 0.688 mol) in acetic acid (860 mL), and 40% HBr solution (825.6 mL) was added a solution of Br₂ (110 g, 0.688 mol) in acetic acid (344 mL) in one portion at 0° C. This mixture was stirred at room temperature overnight, diluted with water (3000 mL), and extracted with 50% Ethyl Acetate/petroleum ether (1500 mL×2). The combined organic layer was washed with a saturated NaHCO₃ solution (1000 mL×2), brine (1000 mL), dried with anhydrous sodium sulfate and concentrated. The residue was purified by column chromatography on silica gel (20% EA/PE) to give the compound I-10b (150 g, yield: 83%) as a white solid. ¹H NMR (400 MHz, CDCl₃) δ 8.85 (dd, J=5.90, 2.89 Hz, 1H), 8.42-8.58 (m, 1H), 7.42 (t, J=9.29 Hz, 1H), 4.52 (d, J=2.01 Hz, 2H).

### Synthesis of I-10c

Into a 500 mL round-bottom flask, was placed 2-bromo-1-(2-fluoro-5-nitrophenyl)ethan-1-one I-10b (30 g, 114.49 mmol, 1 eq.), propan-2-yl N-(2-aminopyrimidin-5-yl)carbamate (11.2 g, 57.08 mmol, 0.5 eq.) and acetone (200 mL). The resulting solution was stirred overnight at 70° C. The reaction mixture was cooled down and the solids were collected by filtration resulting in 15 g (36%) of propan-2-yl N-[2-(2-fluoro-5-nitrophenyl)imidazo[1,2-a]pyrimidin-6-yl]carbamate I-10c as a brown solid.

### Synthesis of I-10

Into a 1 L round-bottom flask was placed tetrahydrofuran (500 mL), Raney Ni (15 g) and propan-2-yl N-[2-(2-fluoro-5-nitrophenyl)imidazo[1,2-a]pyrimidin-6-yl]carbamate I-10c (8 g, 22.26 mmol, 1 eq.). The resulting solution was stirred overnight at room temperature under an atmosphere of hydrogen. The solids were filtered out, and washed with MeOH (4×200 mL). The resulting mixture was concentrated under vacuum to give 7 g (95%) of propan-2-yl N-[2-(5-amino-2-fluorophenyl)imidazo[1,2-a]pyrimidin-6-yl]carbamate I-10 as a brown solid. ¹H NMR (400 MHz, DMSO-d₆) δ 9.94 (s, 1H), 9.24 (s, 1H), 8.46-8.47 (m, 1H), 8.26-8.28 (m, 1H), 7.51-7.53 (m, 1H), 6.96-7.02 (m, 1H), 6.55-6.59 (m, 1H), 4.89-4.98 (m, 1H), 3.17 (s, 2H), 1.07-1.30 (m, 6H). MS m/z=330 (M+1).

### Example 5

### Synthesis of tert-butyl (4-(2-(5-amino-2-chlorophenyl)imidazo[1,2-a]pyrimidin-6-yl)phenyl)(methyl)carbamate (I-11)

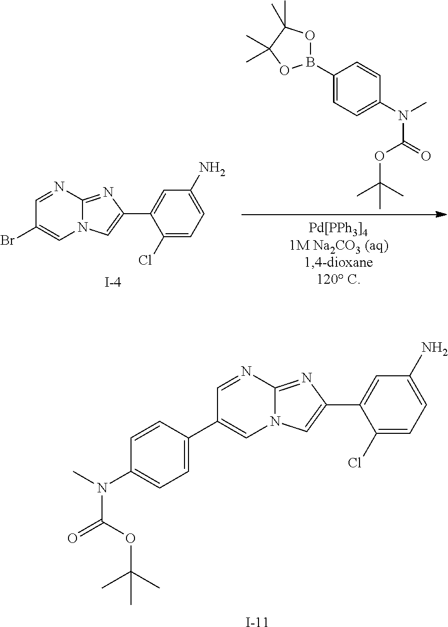

In a 10 mL microwave vial, 3-(6-bromoimidazo[1,2-a]pyrimidin-2-yl)-4-chloroaniline I-4 (140 mg, 0.48 mmol, 1 eq.), tert-butyl methyl(4-(4,4,5,5-tetramethyl-1,3,2-dioxaborolan-2-yl)phenyl)carbamate (216 mg, 0.65 mmol, 1.5 eq.), Tetrakis(triphenylphosphine) palladium (30 mg, 0.02 mmol, 0.05 eq.) and 1 M sodium carbonate solution (0.87 mL, 0.87 mM, 2 eq.) were added. The vial was sealed and purged with nitrogen. 1,4-Dioxane was added into the vial via a syringe and the solution was microwaved for 30 minutes at 120° C. The reaction mixture was diluted with Ethyl Acetate and filtered. Silica gel was added and the solution was evaporated to dryness. Purification was done by flash chromatography, 25% Ethyl Acetate in Hexane isocratically, then via a slow gradient through to 80% Ethyl Acetate. The system was held at 80% to separate tert-butyl (4-(2-(5-amino-2-chlorophenyl)imidazo[1,2-a]pyrimidin-6-yl)phenyl)(methyl)carbamate I-11. ¹H NMR (600 MHz, DMSO-d₆) δ 9.35 (d, J=2.6 Hz, 1H), 8.94 (d, J=2.6 Hz, 1H), 8.50 (s, 1H), 7.75 (d, J=8.7 Hz, 2H), 7.58 (d, J=2.9 Hz, 1H), 7.46 (d, J=8.7 Hz, 2H), 7.17 (d, J=8.5 Hz, 1H), 6.58 (m, 1H), 5.41 (s, 2H), 3.24 (s, 3H), 1.43 (s, 9H). MS m/z=451 (M+1).

### Example 6

### Synthesis 2-methoxyethyl (3-(6-bromoimidazo[1,2-a]pyrimidin-2-yl)-4-chlorophenyl)carbamate (I-12)

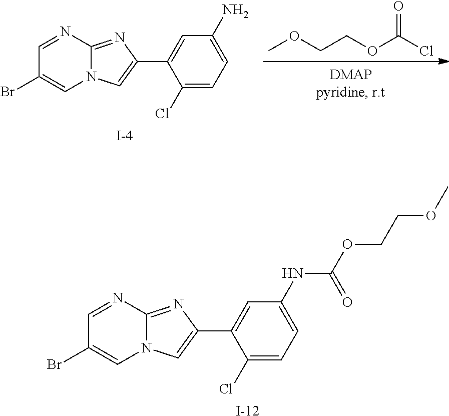

Compound I-4 (0.62 mmol, 1 eq.) was added into a vial with DMAP (1.51 mg, 0.01 mmol, 0.02 eq.) and pyridine (2 mL), and the mixture was flushed with nitrogen. 2-methoxyethyl chloroformate (103 mg, 0.74 mmol, 1.2 eq.) was added and the resulting solution was stirred at room temperature overnight. The reaction mixture was quenched with methanol and evaporated to dryness. Precipitation occurred upon addition of methanol. The precipitate was filtered and dried, and was used for further reactions without purification. MS m/z=426 (M+1).

### Example 7

### Synthesis of methyl (4-(2-(5-amino-2-chlorophenyl)imidazo[1,2-a]pyrimidin-6-yl)phenyl)carbamate (I-13)

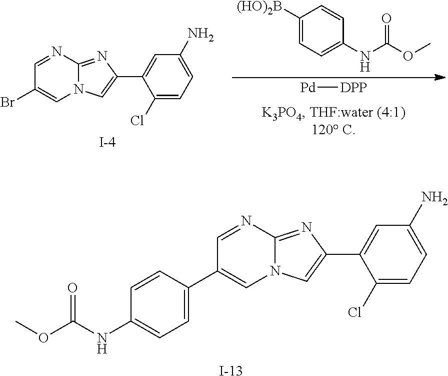

To a solution of 3-(6-bromoimidazo[1,2-a]pyrimidin-2-yl)-4-chloroaniline I-4 (1 eq.) in THF:H₂O (4:1) solvent (0.3 mM) was added (4-((methoxycarbonyl)amino)phenyl) boronic acid (1.5 eq.), K₃PO₄(s) (4 eq.) and DPP-Pd (0.26 mmol/g loading) (0.1 eq.). The vial was sealed and evacuated of air with vacuum and purged with nitrogen. The reaction vial was heated for 1 hour at 120° C. in the microwave oven. The vial was left to cool, and filtered. The filter cake was washed twice with THF:H₂O (4:1) solvent. The filtrate was collected, concentrated in vacuo, and the remaining residue was further purified using column chromatography. ¹H NMR (600 MHz, DMSO-d₆) δ 9.85 (s, 1H), 9.28 (d, J=2.6 Hz, 1H), 8.91 (d, J=2.6 Hz, 1H), 8.48 (s, 1H), 7.71 (d, J=8.8 Hz, 2H), 7.62 (d, J=8.6 Hz, 2H), 7.58 (d, J=2.9 Hz, 1H), 7.16 (d, J=8.5 Hz, 1H), 6.57 (dd, J=2.9 Hz, 8.5, 1H), 5.40 (s, 2H), 3.70 (s, 3H). MS m/z=395 (M+1)

### Example 8

### Synthesis of N-(3-(6-bromoimidazo[1,2-a]pyrimidin-2-yl)-4-chlorophenyl)-5-methylfuran-2-carboxamide (I-14)

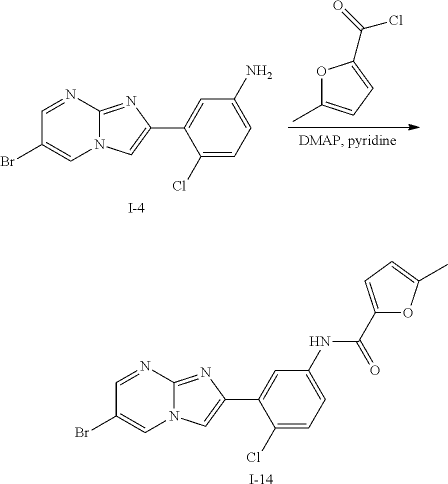

To the 5-methylfuran-2-carbonyl chloride (1.2 eq.) was added a solution of 3-(6-bromoimidazo[1,2-a]pyrimidin-2-yl)-4-chloroaniline I-4 (1 eq.) in anhydrous pyridine (0.3 mM). The resulting solution was stirred and DMAP (0.02 eq.) was added. The reaction was stirred at room temperature overnight. The residue was redissolved in methanol and precipitation yielded N-(3-(6-bromoimidazo[1,2-a]pyrimidin-2-yl)-4-chlorophenyl)-5-methylfuran-2-carboxamide I-14. ¹H NMR (600 MHz, DMSO-d₆) δ 10.30 (s, 1H), 9.38 (d, J=2.5 Hz, 1H), 8.67 (t, J=2.5 Hz, 2H), 8.56 (s, 1H), 7.94 (dd, J=2.7, 8.8 Hz, 1H), 7.54 (d, J=8.7 Hz, 1H), 7.32 (d, J=3.4 Hz, 1H), 6.34 (dd, J=0.9, 3.3 Hz, 1H), 2.39 (s, 3H). MS m/z=431 (M+1)

### Example 9

### Synthesis of N-(3-(6-bromoimidazo[1,2-a]pyrimidin-2-yl)-4-chlorophenyl)cyclopentanecarboxamide (I-15)

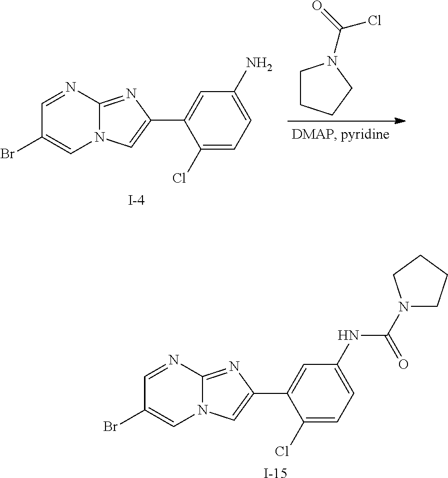

N-(3-(6-bromoimidazo[1,2-a]pyrimidin-2-yl)-4-chlorophenyl)pyrrolidine-1-carboxamide I-15 was synthesized using N-(3-(6-bromoimidazo[1,2-a]pyrimidin-2-yl)-4-chlorophenyl)pyrrolidine-1-carboxamide (I-4) and pyrrolidine-1-carbonyl chloride using the protocol used in the synthesis of I-14. Purification of the compound was done via normal phase column chromatography (30% to 100% Ethyl Acetate/Hexane). ¹H NMR (400 MHz, DMSO-d₆) δ 9.37 (s, 1H), 8.65 (s, 1H), 8.53 (s, 1H), 8.46 (d, J=17.4 Hz, 2H), 7.73 (d, J=8.8 Hz, 1H), 7.41 (d, J=8.8 Hz, 1H), 3.39 (s, 4H), 1.86 (s, 4H). MS m/z=422 (M+1).

### Example 10

### Synthesis of 3-(6-bromoimidazo[1,2-a]pyrimidin-2-yl)-4-fluoroaniline (I-16)

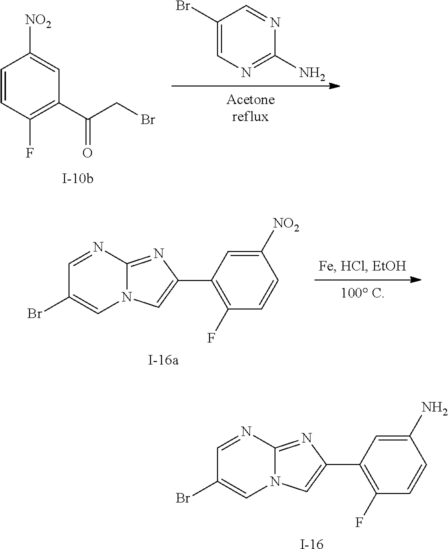

### Synthesis of I-16a

A mixture of compound I-10b (10 g, 57.5 mmol) and 5-bromopyrimidin-2-amine (7.5 g, 28.75 mmol) in acetone (100 mL) was stirred at 80° C. for 12 hours. Additional 5-bromopyrimidin-2-amine (15 g, 57.5 mmol) was then added, and stirred at this temperature for 48 hours. The reaction mixture was concentrated, and the residue was diluted with water (100 mL), and Ethyl Acetate (50 mL). This mixture was filtered and the solid was washed with 0.1 N HCl (50 mL), water (50 mL×3), and dried under vacuum to give compound I-16a (9.8 g, yield: 51%) as a yellow solid. ¹H NMR (400 MHz, DMSO-d₆) δ 9.38 (d, J=2.26 Hz, 1H), 9.04 (dd, J=6.27, 3.01 Hz, 1H), 8.71 (d, J=2.26 Hz, 1H), 8.37 (d, J=4.27 Hz, 1H), 8.34-8.26 (m, 1H), 7.70 (t, J=9.79 Hz, 1H).

### Synthesis of I-16

To a mixture of compound I-16a (7 g, 19.8 mmol) in EtOH (100 mL), and 2N HCl (100 mL) was added Fe (5.5 g, 99 mmol). This reaction mixture was heated to reflux for 30 minutes, cooled down, alkalized with K₂CO₃, diluted with Ethyl Acetate (200 mL), and filtered. The solid was washed with Ethyl Acetate (100 mL). The filtrate was washed with brine, dried with anhydrous Na₂SO₄, and concentrated to give a crude product (300 mg, 70% pure). The solid was washed with hot THF (200 mL×3), and the THF was dried with anhydrous Na₂SO₄, and concentrated to give 3-(6-bromoimidazo[1,2-a]pyrimidin-2-yl)-4-fluoroaniline I-16 (1.6 g, 25%). ¹H NMR (400 MHz, DMSO-d₆) 9.33 (d, J=2.51 Hz, 1H), 8.60 (d, J=2.51 Hz, 1H), 8.15 (d, J=4.02 Hz, 1H), 7.50 (dd, J=6.40, 2.89 Hz, 1H), 6.99 (dd, J=11.29, 8.78 Hz, 1H), 6.56 (dd, J=7.78, 4.27 Hz, 1H), 5.14 (brs, 2H). MS m/z=307 (M+1), 309 (M+3).

### Example 11

### Synthesis of N-(3-(6-bromoimidazo[1,2-a]pyrimidin-2-yl)-4-fluorophenyl)-2,4-dimethyloxazole-5-carboxamide (I-17)

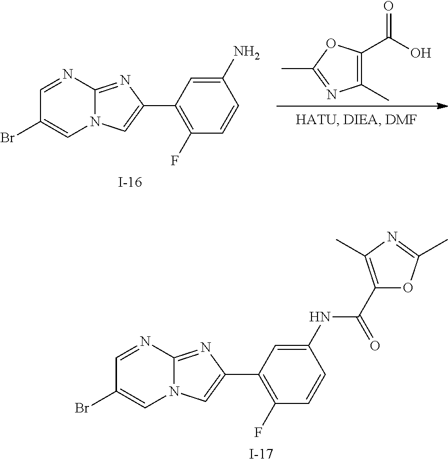

To a solution of 2,4-dimethyloxazole-5-carboxylic acid (4.4 g, 31.3 mmol) in DMF (150 mL) was added DIEA (6.7 g, 52.2 mmol) and HATU (11.9 g, 31.3 mmol). This mixture was stirred at room temperature for 30 minutes, then compound I-16 (8.0 g, 26.1 mmol) was added. This mixture was stirred at room temperature for 4 hours, then water (300 mL) was added and extracted with THF/Ethyl Acetate (500 mL/250 mL), the organic layer was dried with Na₂SO₄ and concentrated under reduced pressure. This crude product was further washed with MeOH (100 mL) to give compound I-17 (6.6 g, 59%) as a yellow solid. ¹H NMR (400 MHz, DMSO-d₆) δ 10.33 (s, 1H), 9.36 (d, J=2.4 Hz, 1H), 8.73 (dd, J=2.4, 6.8 Hz, 1H), 8.66 (d, J=2.4 Hz, 1H), 8.26 (d, J=4 Hz, 1H), 7.86-7.79 (m, 1H), 7.37-7.31 (m, 1H), 2.51 (s, 3H), 2.39 (s, 3H). MS m/z=430 (M+1), 432 (M+3).

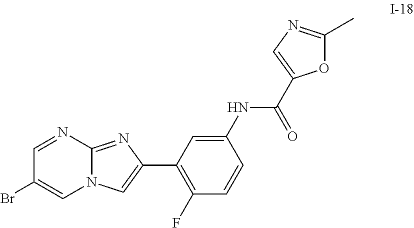 [SMILES: Cc1ncc(C(=O)Nc2ccc(F)c(-c3cn4cc(Br)cnc4n3)c2)o1]

Compound I-18 was prepared following the same protocol as I-17 using 2-methyloxazole-5-carboxylic acid. ¹H NMR (400 MHz, DMSO-d₆) δ 10.50 (s, 1H), 9.36 (d, J=2.4 Hz, 1H), 8.66 (d, J=2.4 Hz, 1H), 8.62 (dd, J=2.8, 6.8 Hz, 1H), 8.26 (d, J=4.4 Hz, 1H), 7.90-7.87 (m, 1H), 7.40-7.34 (m, 1H), 2.54 (s, 3H), 2.51 (s, 3H). MS m/z=415.9 (M+1), 417.9 (M+3).

### Example 12

### Synthesis of isopropyl (3-(6-bromoimidazo[1,2-a]pyrimidin-2-yl)-4-fluorophenyl) carbamate (I-19)

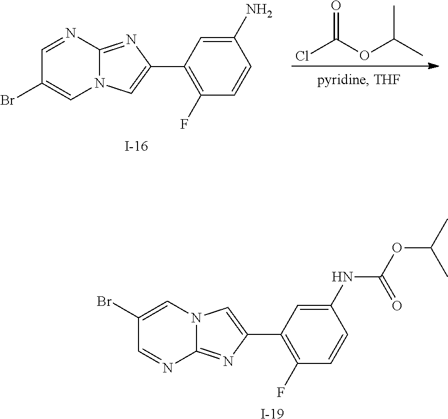

To a solution of compound I-16 (2.5 g, 8.17 mmol) in anhydrous THF (30 mL) was added pyridine (1.9 g, 24.5 mmol) at room temperature. This reaction mixture was stirred at this temperature for 30 minutes, isopropyl chloroformate was added and the mixture was stirred for 3 hours. The reaction mixture was quenched with water (100 mL), filtered and the filter cake was washed with H₂O (50 mL×2) and dried in vacuo to give I-19 (1.2 g, 37.5%) as a yellow solid. ¹H NMR (400 MHz, DMSO-d₆) δ 9.72 (s, 1H), 9.33 (d, J=2.51 Hz, 1H), 8.63 (d, J=2.26 Hz, 1H), 8.41 (d, J=4.27 Hz, 1H), 8.22 (d, J=4.27 Hz, 1H), 7.40-7.54 (m, 1H), 7.26 (dd, J=10.67, 9.16 Hz, 1H), 4.85-4.98 (m, 1H), 1.27 (d, J=6.27 Hz, 6H). MS m/z=393 (M+1), 395 (M+3).

### Example 13

### Synthesis N-(3-(6-bromoimidazo[1,2-a]pyrimidin-2-yl)-4-fluorophenyl) pyrrolidine-1-carboxamide (I-20)

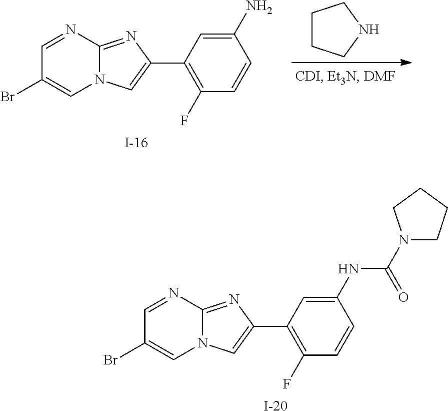

To a solution of CDI (1.2, 3.25 mmol) and Et₃N (0.9 mL, 6.5 mmol) in anh DMF (15 mL) was added compound I-16 (1 g, 3.25 mmol) portion wise at 0° C. This reaction mixture was stirred at this temperature for 3 hours, and pyrrolidine (1.15 g, 16.25 mmol) was added in one portion. The reaction mixture was stirred at room temperature for an hour, diluted with water (30 mL). The solid was collected by the filtration, and washed with water (20 mL), Ethyl Acetate (10 mL), MeOH (10 mL), and dried under vacuum to give compound I-20 (0.65 g, yield: 50%) as a yellow solid. ¹H NMR (400 MHz, DMSO-d₆) δ 9.28 (d, J=2.6 Hz, 1H), 8.57 (d, J=2.5 Hz, 1H), 8.39-8.22 (m, 2H), 8.15 (d, J=4.1 Hz, 1H), 7.61 (ddd, J=8.9, 4.6, 2.8 Hz, 1H), 7.14 (dd, J=11.2, 9.0 Hz, 1H), 3.38-3.30 (m, 4H), 1.85-1.72 (m, 4H). MS m/z=404.5 (M+1), 406.5 (M+3).

### Example 14

### Synthesis of N-(3-(6-bromoimidazo[1,2-a]pyrimidin-2-yl)-4-fluorophenyl)-3-fluoroazetidine-1-carboxamide (I-21)

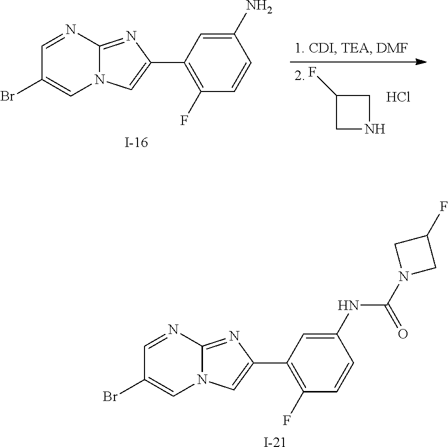

To a solution of CDI (2.62 g, 16.2 mmol) and TEA (4.5 mL, 32.4 mmol) in anhydrous DMF (50 mL) was added compound I-16 (2.5 g, 8.1 mmol) portion wise at 0° C. This reaction mixture was stirred at this temperature for 1 hour, then warmed up to room temperature. 3-Fluoroazetidine hydrochloride (1.8 g, 16.2 mmol) was added an hour later. The resulting reaction mixture was stirred at room temperature for 4 hours, quenched with water (150 mL), and extracted with 50% EA/THF (250 mL×4). The combined organic layers were washed with brine (250 mL×3), dried with anhydrous sodium sulfate, and concentrated in vacuo. The residue was triturated with MeOH (20 mL) to give product I-21 (1.5 g, 50% yield) as a white solid. ¹H NMR (400 MHz, DMSO-d₆) δ 9.34 (s, 1H), 8.84 (s, 1H), 8.63 (s, 1H), 8.35 (dd, J=6.78, 2.76 Hz, 1H), 8.22 (d, J=4.02 Hz, 1H), 7.73-7.59 (m, 1H), 7.24 (dd, J=11.04, 9.03 Hz, 1H), 5.52-5.26 (m, 1H), 4.39-4.22 (m, 2H), 4.10-3.92 (m, 2H). MS m/z=409.8 (M+3).

Intermediates I-22, I-23, I-24, I-25 and I-26 were prepared following the protocol described above using the corresponding commercially available amine.

| No | Structure | ¹HNMR and/or mass |
|---|---|---|
| I-22 | 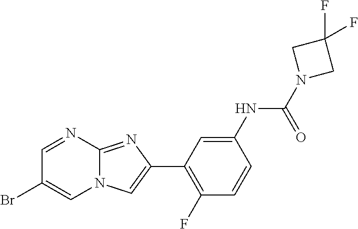 [SMILES: O=C(Nc1ccc(F)c(-c2cn3cc(Br)cnc3n2)c1)N1CC(F)(F)C1] | 1H NMR (400 MHz, DMSO-d₆) δ 9.35 (d, J = 2.51 Hz, 1H), 9.10 (s, 1H), 8.70-8.56 (m, 1H), 8.36 (dd, J = 6.84, 2.82 Hz, 1H), 8.23 (d, J = 4.27 Hz, 1H), 7.68 (ddd, J = 8.97, 4.45, 2.89 Hz, 1H), 7.26 (dd, J = 11.04, 9.03 Hz, 1H), 4.40 (t, J = 12.74 Hz, 4H). MS m/z = 426.0 (M + 1), 428.0 (M + 3). |
| I-23 | 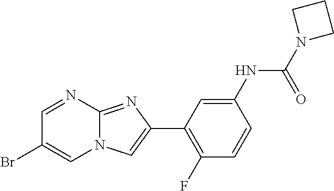 [SMILES: O=C(Nc1ccc(F)c(-c2cn3cc(Br)cnc3n2)c1)N1CCC1] | 1H NMR (400 MHz, DMSO-d₆) δ 9.35 (d, J = 2.51 Hz, 1H), 8.51-8.72 (m, 2H), 8.36 (dd, J = 6.84, 2.70 Hz, 1H), 8.21 (d, J = 4.14 Hz, 1H), 7.74- 7.61 (m, 1H), 7.21 (dd, J = 10.98, 9.10 Hz, 1H), 3.96 (t, J = 7.53 Hz, 4H), 2.18 (q, J = 7.53 Hz, 2H). MS m/z = 390.0 (M + 1), 392.0 (M + 3). |
| I-24 | 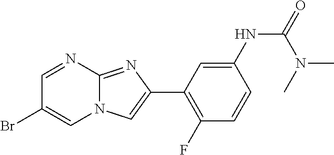 [SMILES: CN(C)C(=O)Nc1ccc(F)c(-c2cn3cc(Br)cnc3n2)c1] | ¹H NMR (400 MHz, CD₃OD) δ 9.16 (d, J = 2.26 Hz, 1H), 8.61 (d, J = 2.26 Hz, 1H), 8.20 (d, J = 3.76 Hz, 1H), 8.00 (dd, J = 6.65, 2.64 Hz, 1H), 7.45-7.59 (m, 1H), 7.10-7.24 (m, 1H), 3.05 (s, 5H). MS m/z = 378.0 (M + 1), 380.0 (M + 3). |
| I-25 | 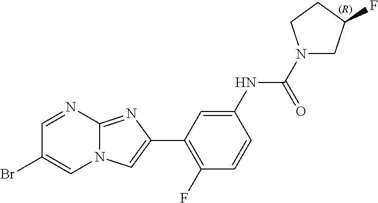 | ¹H NMR (400 MHz, DMSO-d₆) 9.36 (s, 1H), 8.64 (s, 1H), 8.50 (s, 1H), 8.40 (dd, J = 6.78, 2.76 Hz, 1H), 8.23 (d, J = 4.02 Hz, 1H), 7.74-7.61 (m, 1H), 7.24 (dd, J = 11.04, 9.03 Hz, 1H), 5.50-5.27 (m, 1H), 3.83- 3.39 (m, 4H), 2.28-1.98 (m, 2H). MS m/z = 421.9 (M + 1), 423.9 (M + 3). |
| I-26 | 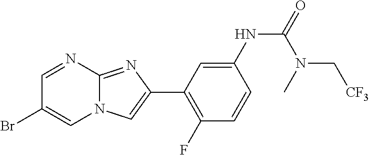 [SMILES: CN(CC(F)(F)F)C(=O)Nc1ccc(F)c(-c2cn3cc(Br)cnc3n2)c1] | ¹H NMR (400 MHz, DMSO-d₆) δ 9.36 (s, 1H), 8.64 (s, 1H), 8.50 (s, 1H), 8.40 (dd, J = 6.78, 2.76 Hz, 1H), 8.23 (d, J = 4.02 Hz, 1H), 7.74- 7.61 (m, 1H), 7.24 (dd, J = 11.04, 9.03 Hz, 1H), 5.50-5.27 (m, 1H), 3.83-3.39 (m, 4H), 2.28-1.98 (m, 2H). MS m/z = 446.0 (M + 1), 448.0 (M + 3). |
| I-27 | 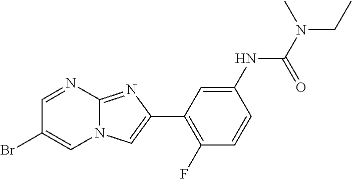 [SMILES: CCN(C)C(=O)Nc1ccc(F)c(-c2cn3cc(Br)cnc3n2)c1] | ¹H NMR (400 MHz, DMSO-d₆) δ 9.34 (d, J = 2.51 Hz, 1H), 8.64 (d, J = 2.51 Hz, 1H), 8.45 (s, 1H), 8.37 (dd, J = 6.90, 2.64 Hz, 1H), 8.21 (d, J = 4.02 Hz, 1H), 7.62-7.59 (m, H), 7.23-7.18 (m, 1H), 2.93 (s, 3H), 1.09 (t, J = 7.03 Hz, 3H). MS m/z = 392.0 (M + 1) |

### Example 15

### Synthesis of (N-(4-fluoro-3-(6-(4,4,5,5-tetramethyl-1,3,2-dioxaborolan-2-yl)imidazo[1,2-a]pyrimidin-2-yl)phenyl)-2,4-dimethyloxazole-5-carboxamide (I-28)

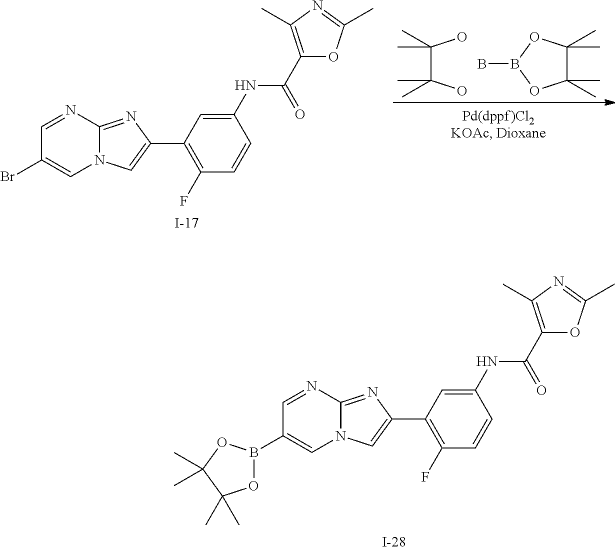

To a solution of compound I-17 (15 g, 34.9 mmol) in 1,4-dioxane (300 mL) was added 4,4,4′,4′,5,5,5′,5′-octamethyl-2,2′-bi(1,3,2-dioxaborolane) (35 g, 140 mmol), Pd(dppf)Cl₂ (2.5 g, 3.49 mmol) and KOAc (10.2 g, 105 mmol) under N₂. This mixture was heated to 100° C. for 16 hours. The solvent was removed, then the residue was redissolved in THF and filtered over silica gel. The solvent was concentrated and the residue triturated with MTBE to give compound I-28 (10 g, 60%). ¹H NMR (400 MHz, DMSO-d₆) δ 10.34 (s, 1H), 9.30 (d, J=2.01 Hz, 1H), 8.72 (dd, J=6.90, 2.64 Hz, 1H), 8.62 (d, J=2.01 Hz, 1H), 8.32-8.37 (m, 1H), 7.77-7.85 (m, 1H), 7.33 (dd, J=10.92, 9.16 Hz, 1H), 2.49-2.51 (m, 3H), 2.41 (s, 3H), 1.36 (s, 12H). MS m/z=396.1 (M+1).

### Example 16

### Synthesis of tert-butyl (2-(5-amino-2-fluorophenyl)imidazo[1,2-a]pyrimidin-6-yl)carbamate (I-29)

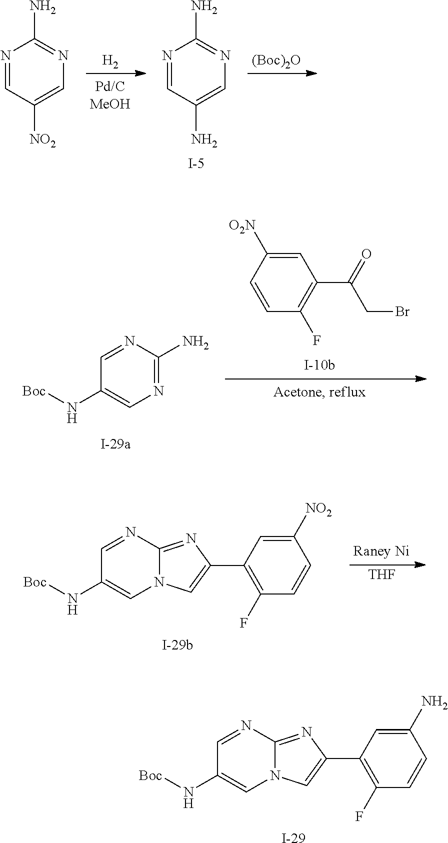

### Synthesis of compound I-29a

A mixture of 5-nitropyrimidin-2-amine (50 g, 0.357 mol) and Pd/C (10 g) in MeOH (800 mL) was stirred with a balloon of H₂ at room temperature for 18 hours. The reaction mixture was degassed with N₂ three times, cooled to 0° C., and (Boc)₂O (74 g, 0.34 mol) was added. This reaction mixture was stirred at this temperature for 6 hours and filtered. The cake was washed with Ethyl Acetate (300 mL×3), and the filtrate was concentrated at 40° C. to give a yellow solid. This crude solid was purified by column chromatography on silica gel (DCM: EA=1:2) to give compound I-29a (50 g, yield: 67%) as a yellow solid. ¹H NMR (400 MHz, CDCl₃) δ 8.34 (brs, 2H), 6.21 (brs, 1H), 4.98 (brs, 2H), 1.50 (s, 9H).

### Synthesis of compound I-29b

A mixture of tert-butyl (2-aminopyrimidin-5-yl)carbamate I-29a (10 g, 47.6 mmol) and 2-bromo-1-(2-fluoro-5-nitrophenyl) ethanone I-10b (25 g, 95.2 mmol) in acetone (20 mL) was stirred under reflux for 20 hours. The reaction mixture was cooled down, and filtered. The solid was washed with acetone (10 mL×2), and dried under vacuum to give compound I-29b (9 g, 50% yield) as a white solid. ¹H NMR (400 MHz, DMSO-d₆) δ 9.97 (brs, 1H), 9.43 (brs, 1H), 9.03 (dd, J=6.40, 2.89 Hz, 1H), 8.58 (dd, J=7.03, 3.51 Hz, 2H), 8.39-2.21 (m, 1H), 7.69 (s, 1H), 1.52 (s, 9H).

### Synthesis of compound I-29

A mixture of I-29b (9 g, 24.1 mmol) and Raney Ni (18 g) in anhydrous THF (800 mL) was stirred at room temperature under a balloon of H₂ for 12 hours. This reaction mixture was diluted with MeOH (800 mL), and filtered. The cake was washed with CH₃OH (300 mL×3), and the filtrate was concentrated under vacuum at 35-40° C. to give compound I-29 (8.2 g, 90% pure). ¹H NMR (400 MHz, DMSO-d₆) δ 9.79 (brs, 1H), 9.29 (brs, 1H), 8.45 (d, J=2.51 Hz, 1H), 8.25 (d, J=4.02 Hz, 1H), 7.47 (dd, J=6.27, 2.76 Hz, 1H), 6.96 (dd, J=11.04, 8.78 Hz, 1H), 6.60-6.47 (m, 1H), 5.12 (brs, 2H), 1.51 (s, 9H). MS m/z=344.41 (M+1).

### Example 17

### Synthesis of N-(3-(6-aminoimidazo[1,2-a]pyrimidin-2-yl)-4-fluorophenyl)-5-fluorofuran-2-carboxamide (I-30)

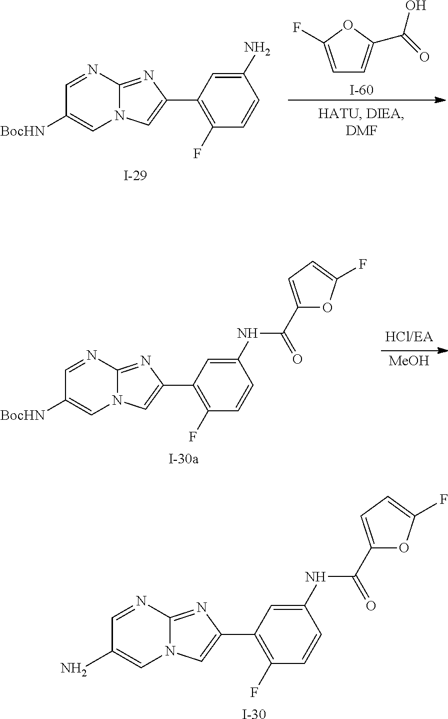

### Synthesis of I-30a

To a solution of 5-fluorofuran-2-carboxylic acid I-60 (1.137 g, 8.75 mmol) in DMF (10 mL), HATU (4.985 g, 13.1 mmol) and DIEA (3.385 g, 26.2 mmol) were added. 20 minutes later, compound I-29 (3 g, 8.75 mmol) was added. The mixture was stirred at room temperature overnight, diluted with water (150 mL), extracted with Ethyl Acetate (80 mL×2), washed with a saturated sodium bicarbonate solution (80 mL), dried and concentrated under reduced pressure. The residue was purified by chromatography (50% EA/PE) to give compound I-30a (2.2 g, 58%) as a white solid. ¹H NMR (400 MHz, DMSO-d₆) δ 10.34 (s, 1H), 9.81 (brs, 1H), 9.32 (brs., 1H), 8.58 (dd, J=6.90, 2.76 Hz, 1H), 8.49 (d, J=2.76 Hz, 1H), 8.37 (d, J=4.14 Hz, 1H), 7.82 (ddd, J=8.91, 4.39, 2.89 Hz, 1H), 7.45 (t, J=3.58 Hz, 1H), 7.31 (dd, J=10.92, 9.03 Hz, 1H), 6.11 (dd, J=7.15, 3.64 Hz, 1H), 1.52 (s, 9H).

### Synthesis of I-30

To a solution of compound I-30a (2.1 g, 4.6 mmol) in methanol (210 mL), hydrochloric acid in Ethyl Acetate (80 mL, 4M) was added dropwise. The reaction mixture was stirred for 2 hours, concentrated, washed with a saturated sodium bicarbonate solution (50 mL) and water (100 mL), and dried to give compound I-30 (1.2 g, 73%) as a yellow solid. ¹H NMR (400 MHz, DMSO-d₆) δ 8.34 (d, J=2.76 Hz, 1H), 8.16-8.11 (m, 2H), 8.04 (d, J=4.02 Hz, 1H), 7.87 (ddd, J=8.91, 4.39, 2.89 Hz, 1H), 7.32 (t, J=3.51 Hz, 1H), 7.23 (dd, J=10.92, 8.91 Hz, 1H), 5.91 (dd, J=7.03, 3.64 Hz, 1H). MS m/z=356.0 (M+1).

### Compound I-31

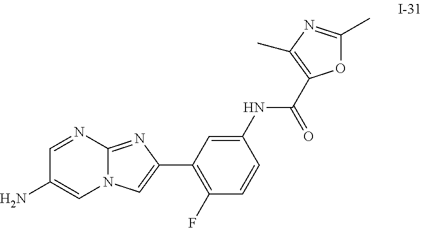 [SMILES: Cc1nc(C)c(C(=O)Nc2ccc(F)c(-c3cn4cc(N)cnc4n3)c2)o1]

Compound I-31 was prepared following the protocol described above using 2,4-dimethyloxazole-5-carboxylic acid. ¹H NMR (400 MHz, DMSO-d₆) δ 10.45 (s, 1H), 8.62 (d, J=2.8 Hz, 1H), 8.50-8.46 (m, 2H), 8.40 (d, J=2.8 Hz, 1H), 7-77-7.73 (m, 1H), 7.46 (t, J=9.2 Hz, 1H), 2.50 (s, 3H), 2.39 (s, 3H). MS m/z=367.1 (M+1).

### Example 18

### Synthesis of (R)—N-(3-(6-aminoimidazo[1,2-a]pyrimidin-2-yl)-4-fluorophenyl)-3-fluoropyrrolidine-1-carboxamide (I-32)

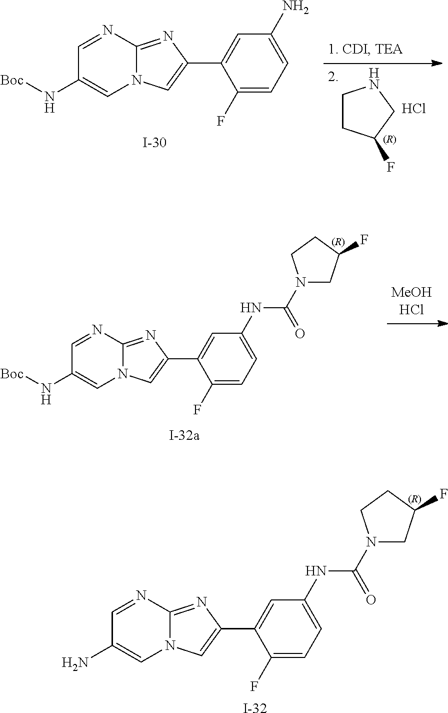

### Synthesis of I-32a

To a solution of DMF (30 mL) was added CDI (0.97 g, 6 mmol), TEA (1.65 mL) and compound I-30 (1.02 g, 3 mmol) at 0° C. This reaction mixture was stirred at 0° C. for 2 hours, and allowed to warm to room temperature and stirred for another 2 hours. The (R)-3-fluoropyrrolidine hydrochloride (755 mg, 6 mmol) was added to the flask, and stirred overnight. The reaction mixture was diluted with water (100 mL), extracted with 50% EA/THF (30 mL×3). The combined organic layers were washed with brine (20 mL×2), dried over anhydrous sodium sulfate, and concentrated. The residue was tritutated with MeOH (10 mL) to give compound I-32a (1.2 g, yield, 90%). ¹H NMR (400 MHz, DMSO-d₆) δ 9.79 (s, 1H), 9.31 (s, 1H), 8.48 (d, J=2.76 Hz, 2H), 8.40-8.21 (m, 2H), 7.63 (dt, J=7.97, 3.92 Hz, 1H), 7.20 (dd, J=10.67, 9.16 Hz, 1H), 5.55-5.23 (m, 1H), 3.81-3.40 (m, 4H), 2.29-1.93 (m, 2H), 1.52 (s, 9H).

### Synthesis of I-32

A mixture of I-32a (1.3 g, 12.6 mmol) in 2N MeOH/HCl (30 mL) was stirred at room temperature overnight. The reaction mixture was concentrated under vacuum, quenched with a saturated. sodium bicarbonate solution (50 mL). The solid was collected by filtration, washed with water (50 mL), MeOH (5 mL×2), and dried under vacuum to give I-32 (700 mg, yield: 78%) as a pale solid. ¹H NMR (400 MHz, DMSO-d₆) δ 8.44 (s, 1H), 8.29 (d, J=7.2, 1H), 8.22 (s, 1H), 8.07-8.10 (m, 2H), 7.57-7.60 (m, 1H), 7.13-7.18 (m, 1H), 5.37 (d, J=7.2, 52.8, 1H), 5.21 (s, 1H), 3.63-3.72 (m, 4H), 2.14-2.19 (m, 2H). MS m/z=359.1 (M+1).

### Example 19

### Synthesis of tert-butyl (2-(2-fluoro-5-(pyrrolidine-1-carboxamido)phenyl)imidazo[1,2-a]pyrimidin-6-yl)carbamate (I-33)

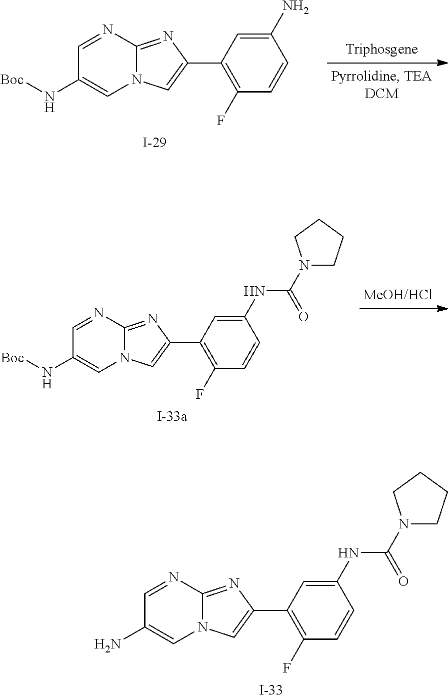

### Synthesis of I-33a

In a 50 mL round bottom flask containing the intermediate I-29 (1.019 mmol, 350 mg) and triphosgene (1.121 mmol, 333 mg) in DCM cooled down to 0° C., triethylamine (1.529 mmol, 213 μl) was added. The reaction mixture was stirred at 0° C. for 15 minutes. At this stage, pyrrolidine (3.57 mmol, 295 μl) was slowly added and the mixture was brought to room temperature and stirred for 16 hours. The reaction mixture was diluted with water and DCM and transferred into a separating funnel. The organic layer was recovered and after drying over Na₂SO₄, was adsorbed onto silica gel. Flash column chromatography (Cyclohexane/Ethyl Acetate) allowed the isolation of the desired product as a white solid (140 mg, 30%). ¹H NMR (400 MHz, DMSO-d₆) δ 9.77 (brs, 1H), 9.29 (brs, 1H), 8.47 (d, J=2.69 Hz, 1H), 8.34 (dd, J=6.91, 2.87 Hz, 1H), 8.30-8.32 (m, 2H), 7.63 (ddd, J=8.96, 4.43, 2.87 Hz, 1H), 7.17 (dd, J=11.00, 9.05 Hz, 1H), 3.39 (t, J=6.60 Hz, 4H), 1.83-1.89 (m, 4H), 1.51 (s, 9H). MS m/z=441.2 (M+1).

### Synthesis of I-33

Compound I-33 was prepared following the protocol described in Example 18 to prepare I-32. ¹H NMR (400 MHz, DMSO-d₆) δ 8.59 (d, J=2.51 Hz, 1H), 8.44 (s, 2H), 8.36 (d, J=2.51 Hz, 1H), 8.22 (dd, J=6.78, 2.51 Hz, 1H), 7.57-7.53 (m, 1H), 7.37-7.32 (m, 1H), 3.40-3.38 (m, 4H), 1.87-1.89 (m, 4H). MS m/z=341.2 (M+1).

### Example 20

### Synthesis of 4-fluoro-3-(6-(pyridin-2-yl)imidazo[1,2-a]pyrimidin-2-yl)aniline hydrochloride (I-34)

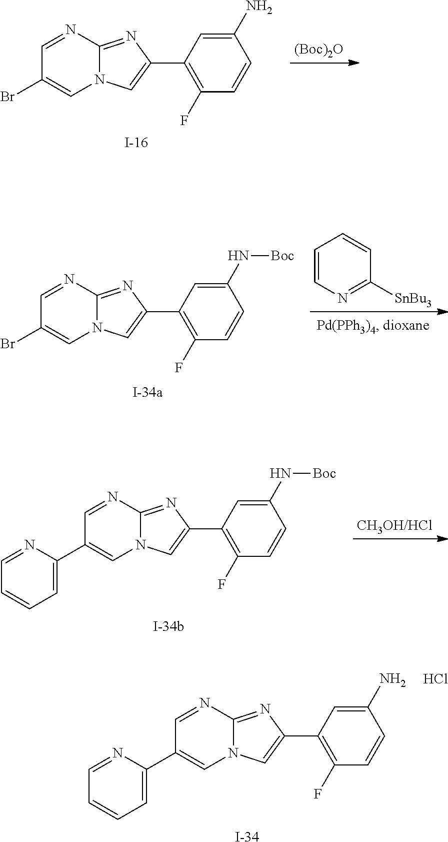

### Synthesis of I-34a

A mixture of compound I-16 (5 g, 16.3 mmol), and di-tert-butyl dicarbonate (20 g, 81.4 mmol) in THF (50 mL) was stirred under reflux for 24 hours. The reaction mixture was concentrated, and the residue was washed with Petroleum Ether. The yellow solid (6 g, yield: 90%) was dried under vacuum and used directly in the next step. MS m/z=407.0 (M+1), 409.0 (M+3).

### Synthesis of I-34b

A mixture of compound I-34a (4 g, 9.82 mmol), and 2-(tributylstannyl)pyridine (18.1 g, 49.11 mmol) in 1,4-dioxane (100 mL) was stirred at room temperature under N₂. Tetrakis(triphenylphosphine)palladium (567 mg, 0.49 mmol) was added and the reaction mixture was heated to 110° C. overnight. The solvent was evaporated and the residue was dissolved in THF (200 mL). The solution was passed through a short silca gel column, and the column was washed with THF (100 mL×3). The combined THF was concentrated under vacuo. The residue was crystallized with MeOH (30 mL) to give product I-34b (1.7 g, yield: 42.5%) as a yellow solid. MS m/z=406.1 (M+1).

### Synthesis of I-34

A mixture of compound I-34b (2 g, 5 mmol) in 2N MeOH (50 mL) was stirred at room temperature overnight. The reaction was concentrated to 10 mL, and filtered. The solid was dried in vacuo to give I-34 (1.7 g, yield: 85%) as a white solid. ¹H NMR (400 MHz, DMSO-d₆) δ 9.80 (s, 1H), 9.36 (s, 1H), 8.76 (d, J=4.27 Hz, 1H), 8.50 (d, J=4.02 Hz, 1H), 8.32 (dd, J=6.27, 2.51 Hz, 1H), 8.17-8.11 (m, 1H), 8.05 (td, J=7.72, 1.63 Hz, 1H),7.59-7.41 (m, 3H). MS m/z=306.0 (M+1).

### Example 21

### Synthesis of 4-fluoro-3-(6-fluoroimidazo[1,2-a]pyrimidin-2-yl)aniline (I-35)

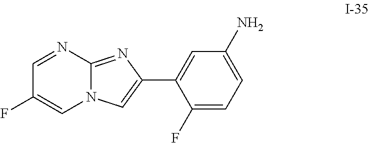 [SMILES: Nc1ccc(F)c(-c2cn3cc(F)cnc3n2)c1]

Compound I-35 was prepared following the same synthetic route used to prepared compound I-10 using 5-fluoropyrimidin-2-amine as starting material. ¹H NMR (400 MHz, DMSO-d₆) δ 9.23 (brs, 1H), 8.74 (brs, 1H), 8.20 (brs, 1H), 7.51 (brs, 1H), 6.56 (brs, 1H), 5.13 (brs, 2H). MS m/z=247.1 (M+1).

### Example 22

### Synthesis of 4-fluoro-3-(6-(trifluoromethyl)imidazo[1,2-a]pyrimidin-2-yl)aniline (I-36)

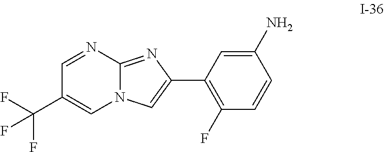 [SMILES: Nc1ccc(F)c(-c2cn3cc(C(F)(F)F)cnc3n2)c1]

Compound I-36 was prepared following the same synthetic route used to prepared compound I-10 using 5-(trifluoromethyl)pyrimidin-2-amine as starting material. ¹H NMR (400 MHz, DMSO-d₆) δ 9.65 (s, 1H), 8.87 (s, 1H), 8.32 (s, 1H), 7.54 (s, 1H), 7.00-7.02 (m, 1H), 6.59 (s, 1H), 5.17 (s, 2H).

### Example 23

### Synthesis of 4-fluoro-3-(6-(trifluoromethyl)imidazo[1,2-a]pyrimidin-2-yl)aniline (I-37)

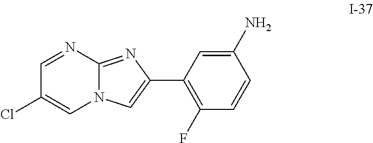 [SMILES: Nc1ccc(F)c(-c2cn3cc(Cl)cnc3n2)c1]

Compound I-37 was prepared following the same synthetic route used to prepared compound I-10 using 5-chloropyrimidin-2-amine as starting material. ¹H NMR (400 MHz, DMSO-d₆) δ 9.28 (s, 1H), 8.59 (s, 1H), 8.17 (s, 1H), 7.51 (dd, J=6.27, 2.76 Hz, 1H), 7.00 (dd, J=11.17, 8.91 Hz, 1H), 6.64-6.50 (m, 1H), 5.14 (s, 2H). MS m/z=262.9 (M+1).

### Example 24

### Synthesis of isopropyl (2-(5-bromo-2-fluorophenyl)imidazo[1,2-a]pyrimidin-6-yl)carbamate (I-38)

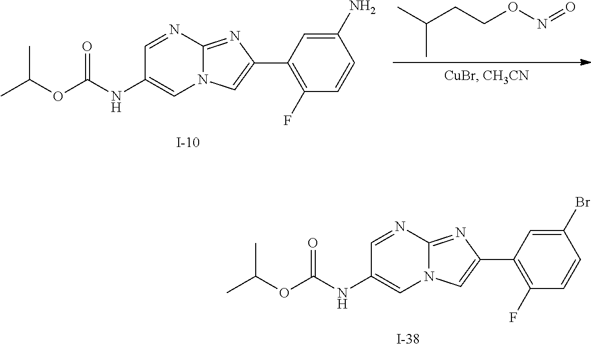

To a mixture of CuBr (0.9 g, 6.7 mmol) in CH₃CN (20 mL) was added isopentyl nitrite (1.2 g, 9.9 mmol), and compound I-10 (1.1 g, 3.3 mmol) portion wise. This mixture was stirred at room temperature for 2 hours, and diluted with NH₄OH (30 mL) and Ethyl Acetate (20 mL). The reaction mixture was stirred for 20 min, and filtered. The solid was washed with water (10 mL×3), Ethyl Acetate (10 mL), and dried under vacuum to give I-38 (700 mg, yield: 53%) as a yellow solid. ¹H NMR (400 MHz, DMSO-d₆) δ 10.02 (brs, 1H), 9.30 (brs, 1H), 8.51 (d, J=2.26 Hz, 1H), 8.43 (d, J=4.02 Hz, 1H), 8.34 (dd, J=6.65, 2.38 Hz, 1H), 7.62-7.52 (m, 1H), 7.41-7.29 (m, 1H), 4.99-4.80 (m, 1H), 1.29 (d, J=6.27 Hz, 6H). MS m/z=393.0 (M+1), 395.0 (M+3).

### Example 25

### Synthesis of N-(2-(5-amino-2-fluorophenyl)imidazo[1,2-a]pyrimidin-6-yl)acetamide (I-39)

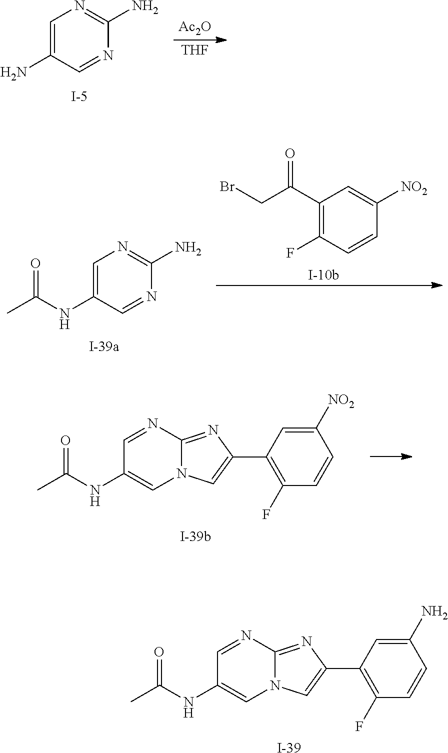

### Synthesis of I-39a

To a solution of 1-5 in THF (0.3M) was added dropwise acetic anhydride (1.2 eq.) at room temperature and was allowed to stir for 1 hour. The reaction was diluted with methanol, dried in vacuo, azetroped with toluene, and used directly in the next step. ¹H NMR (400 MHz, DMSO-d₆) δ 9.71 (s, 1H), 8.33 (d, J=2.2 Hz, 2H), 6.44 (s, 2H), 1.99 (s, 3H).

### Synthesis of I-39b

Compound I-39b was prepared following the procedure described for the synthesis of I-10c using I-39a as starting material. ¹H NMR (400 MHz, DMSO-d₆) δ 10.53 (s, 1H), 9.67 (d, J=2.4 Hz, 1H), 9.03 (d, J=3.4 Hz, 1H), 8.70-8.55 (m, 2H), 8.29 (s, 1H), 7.69 (t, J=9.7 Hz, 1H), 2.14 (s, 3H).

### Synthesis of I-39

Compound I-39 was prepared following the procedure described for the synthesis of 1-4 using I-39b as starting material. ¹H NMR (600 MHz, DMSO-d₆) 10.51 (s, 1H), 9.58 (d, J=2.6 Hz, 1H), 8.55 (d, J=2.6 Hz, 1H), 8.43 (d, J=4.0 Hz, 2H), 7.95 (s, 1H), 7.30 (s, 1H), 7.08 (s, 1H), 2.53 (t, J=5.6 Hz, 3H).

### Example 26

### Synthesis of N-(4-fluoro-3-(6-isothiocyanatoimidazo[1,2-a]pyrimidin-2-yl)phenyl)-2,4-dimethyloxazole-5-carboxamide (I-40)

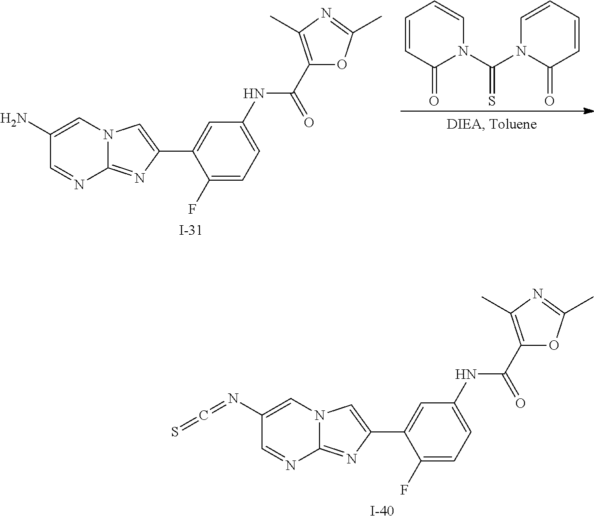

A mixture of I-31 (0.4 g, 1.09 mmol), 1,1′-thiocarbonylbis(pyridin-2(1H)-one) (0.38 g, 1.63 mmol) and DIEA (0.286 mL, 1.63 mmol) was refluxed in toluene overnight. The reaction mixture was cooled down to room temperature, filtered and evaporated to dryness. Purification by flash chromatography (0 to 100% Ethyl Acetate in Hexanes) gave the intermediate I-40. ¹H NMR (400 MHz, DMSO-d₆) δ 10.36 (s, 1H), 9.28 (d, J=2.6 Hz, 1H), 8.73 (dd, J=2.7, 5.6 Hz, 2H), 8.29 (d, J=4.1 Hz, 1H), 7.82 (ddd, J=2.8, 4.5, 8.9 Hz, 1H), 7.35 (dd, J=9.0, 11.0 Hz, 1H). MS m/z=409.2 (M+1).

### Synthesis of isopropyl (2-(2-fluoro-5-isothiocyanatophenyl)imidazo[1,2-a]pyrimidin-6-yl)carbamate (I-41)

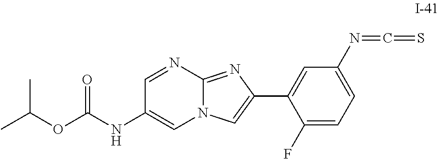 [SMILES: CC(C)OC(=O)Nc1cnc2nc(-c3cc(N=C=S)ccc3F)cn2c1]

Compound I-41 was prepared following the procedure described for the synthesis of 1-40 using I-10 as starting material. ¹H NMR (400 MHz, DMSO-d₆) 10.10 (s, 1H), 9.37 (s, 1H), 8.58 (d, J=2.7 Hz, 1H), 8.51 (d, J=4.3 Hz, 1H), 8.31-8.19 (m, 1H), 7.58-7.42 (m, 2H), 5.08-4.90 (m, 1H), 1.36 (s, 3H), 1.35 (s, 3H). MS m/z=372.1 (M+1).

### Example 27

### Synthesis of N-(3-(6-aminoimidazo[1,2-a]pyrimidin-2-yl)-4-fluorophenyl)-2-methyloxazole-5-carboxamide (I-42)

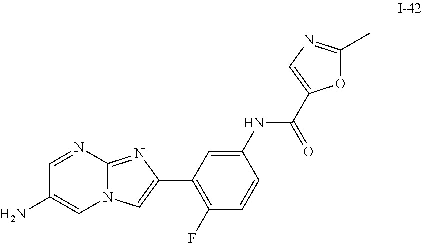 [SMILES: Cc1ncc(C(=O)Nc2ccc(F)c(-c3cn4cc(N)cnc4n3)c2)o1]

Compound I-42 was prepared following the protocol described in Example 17 using 2-methyloxazole-5-carboxylic acid. ¹H NMR (400 MHz, DMSO-d₆) δ 10.47 (s, 1H), 8.59-8.54 (m, 1H), 8.29-8.12 (m, 3H), 7.90 (s, 1H), 7.84-7.80 (m, 1H), 7.33-7.28 (m, 1H), 5.32-5.16 (m, 2H), 2.54 (s, 3H). MS m/z=353.1 (M+1).

### Example 28

### Synthesis of N-(3-(6-aminoimidazo[1,2-a]pyrimidin-2-yl)-4-fluorophenyl)-3,3-difluoroazetidine-1-carboxamide (I-43)

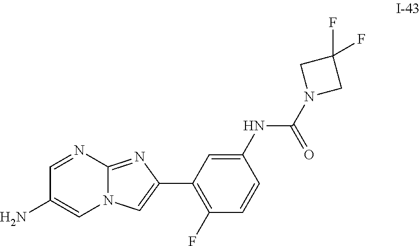 [SMILES: Nc1cnc2nc(-c3cc(NC(=O)N4CC(F)(F)C4)ccc3F)cn2c1]

Compound I-43 was prepared following the protocol described in Example 18 using 3,3-difluoroazetidine hydrochloride. MS m/z=363.1 (M+1).

### Example 29

### Synthesis of N-(3-(6-bromoimidazo[1,2-a]pyrimidin-2-yl)phenyl)-2,4-dimethyloxazole-5-carboxamide (I-44)

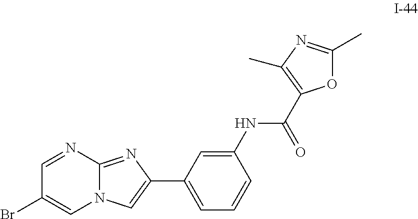 [SMILES: Cc1nc(C)c(C(=O)Nc2cccc(-c3cn4cc(Br)cnc4n3)c2)o1]

Compound I-44 was prepared following the synthetic route described for the preparation of compound I-17 using 2-bromo-1-(3-nitrophenyl)ethanone as starting material. ¹H NMR (DMSO-d₆, 400 MHz) 10.26 (s, 1H), 9.34 (s, 1H), 8.60 (d, J=4.0 Hz, 1H), 8.48 (s, 1H), 8.29 (s, 1H), 7.74 (t, J=8.0 Hz, 2H), 7.44 (t, J=8.0 Hz, 1H), 2.40 (s, 3H).

### Example 30

### Synthesis of (R)—N-(3-(6-bromoimidazo[1,2-a]pyrimidin-2-yl)phenyl)-3-fluoropyrrolidine-1-carboxamide (I-45)

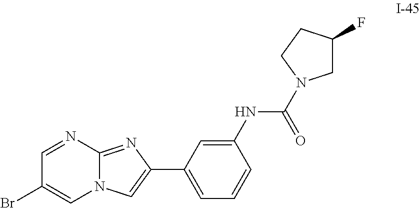 [SMILES: O=C(Nc1cccc(-c2cn3cc(Br)cnc3n2)c1)N1CC[C@@H](F)C1]

Compound I-45 was prepared following the synthetic route described for the preparation of compound I-25 using 2-bromo-1-(3-nitrophenyl)ethanone as starting material. MS m/z=404.0 (M+1), 406.0 (M+3).

### Example 31

### Synthesis of isopropyl ((2-(5-amino-2-fluorophenyl)imidazo[1,2-a]pyrimidin-6-yl)methyl)carbamate

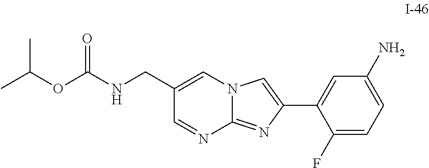 [SMILES: CC(C)OC(=O)NCc1cnc2nc(-c3cc(N)ccc3F)cn2c1]

Compound I-46 was prepared using the same synthetic route as intermediate I-10 starting with 5-(aminomethyl)pyrimidin-2-amine. MS m/z=344.0 (M+1)

### Example 32

### Synthesis of 3-(6-(difluoromethyl)imidazo[1,2-a]pyrimidin-2-yl)-4-fluoroaniline (I-47)

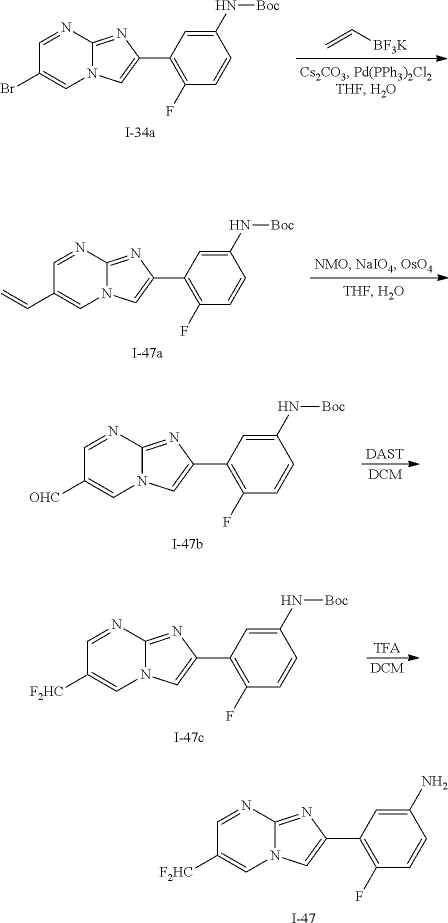

### Synthesis of I-47a

A mixture of compound I-34a (6 g, 15 mmol), and potassium vinyltrifluoroborate (6.8 g, 4.5 mmol) and cesium carbonate (14.67 g, 45 mmol) in THF (150 mL) and water (15 mL) was stirred at room temperature under nitrogen. To this reaction mixture was added Bis(triphenylphosphine)palladium(II) dichloride (525 mg, 0.75 mmol), and the mixture was heated to 80° C. overnight under nitrogen. The reaction mixture was cooled down and diluted with Ethyl Acetate (150 mL). The organic layer was washed with brine, dried with anhydrous sodium sulfate, and concentrated in vacuo. The residue was triturated with MeOH (20 mL) and stirred for 20 minutes. The solid was collected by filtration, and dried in vacuo to give the crude compound I-47a (2 g, yield: 38%) as a yellow solid. ¹H NMR (400 MHz, DMSO-d₆) δ 9.51 (s, 1H), 9.05 (s, 1H), 8.89 (s, 1H), 8.22 (d, J=4.02 Hz, 1H), 7.39-7.51 (m, 3H), 7.24 (dd, J=10.92, 9.16 Hz, 1H), 6.76 (dd, J=17.69, 11.17 Hz, 1H), 6.04 (d, J=17.82 Hz, 1H), 5.44 (d, J=11.29 Hz, 1H), 1.51 (s, 9H). MS m/z=355.0 (M+1)

### Synthesis of I-47b

To a solution of compound I-47a (2 g, 5.6 mmol) in THF (200 mL) and water (20 mL) was added NMO (1.3 g, 11.2 mmol) and osmium (VIII) oxide (71.7 mg, 0.28 mmol) at room temperature. After 2 hours, sodium periodate (1.3 g, 11.2 mmol) was added and the reaction mixture was stirred at room temperature overnight. The mixture was quenched with sodium hydrogen sulfite (200 mL) at 0° C., and stirred for 30 minutes. The reaction mixture was then extracted with 50% EA/THF (200 mL×3). The combined organic layers were washed with brine (200 mL), dried with anhydrous sodium sulfate, and concentrated in vacuo. The residue was triturated with MeOH (20 mL), and the solid was collected by filtration to give compound I-47b (1.3 g, yield: 62%) as a yellow solid. ¹H NMR (400 MHz, DMSO-d₆) δ 9.64 (s, 1H), 10.04 (s, 1H), 9.54 (s. 1H), 8.97 (d, J=2.26 Hz, 1H), 8.40-8.56 (m, 2H), 7.39-7.55 (m, 1H), 7.28 (dd, J=10.67, 9.16 Hz, 1H), 1.51 (s, 1H). MS m/z=357.0 (M+1).

### Synthesis of I-47c

To a solution of compound I-47b (1.3 g, 3.65 mmol) in anhydrous DCM (200 mL) was added DAST (1.8 g, 11 mmol) and the resulting solution was stirred at room temperature over 2 days. The mixture was quenched with sodium bicarbonate solution (50 mL) at 0° C. The DCM was removed, and the residue was extracted with 50% EA/THF (200 mL×3). The combined organic layers were washed with brine (200 mL), dried over anhydrous sodium sulfate, and concentrated in vacuo. The residue was purified by silica gel chromatography (DCM: MeOH/100:3) and the residue was triturated with DCM (5 mL) to give compound I-47c (600 mg, yield: 40%) as a yellow solid. ¹H NMR (400 MHz, DMSO-d₆) δ 9.52 (s, 1H), 9.35 (s, 1H), 8.76 (s, 1H), 8.49 (d, J=4.27 Hz, 1H), 8.38 (d, J=4.02 Hz, 1H), 7.13-7.74 (m, 3H), 1.50 (s, 9H). MS m/z=379.0 (M+1).

### Example 33

### Synthesis of N-(3-(6-bromo-3-fluoroimidazo[1,2-a]pyrimidin-2-yl)-4-fluorophenyl)-2,4-dimethyloxazole-5-carboxamide (I-48)

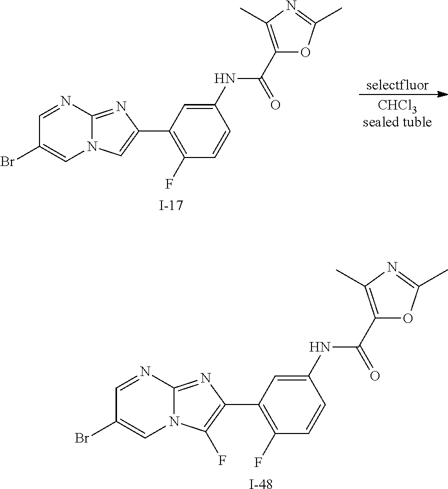

A mixture of I-17 (1 g, 2.3 mmol) and SelectFluor (1.24 g, 3.49 mmol) in chloroform (60 mL) was allowed to stir in a sealed tube at 90° C. An addition of SelectFluor (1.24 g, 3.49 mmol) was added each day for 2 days, and the reaction was stirred and monitored for another 2 days. The reaction mixture was concentrated in vacuo and the residue was redissolved in Ethyl Acetate (200 mL). The Ethyl Acetate was washed with brine (100 mL), dried with anhydrous sodium sulfate, and concentrated under vacuum. The residue was purified by HPLC to give I-48 (250 mg, yield, 30%) as a yellow solid. ¹H NMR (400 MHz, DMSO-d₆) δ 10.34 (s, 1H), 9.27 (s, 1H), 8.65 (s, 1H), 8.45 (dd, J=6.65, 2.64 Hz, 1H), 7.86 (td, J=4.52, 3.01 Hz, 1H), 7.42-7.26 (m, 1H), 2.5 (s, 3H), 2.39 (s, 3H). MS m/z=447.9 (M+1), 450.0 (M+3).

### Example 34

### Synthesis of 3-(6-cyclobutylimidazo[1,2-a]pyrimidin-2-yl)-4-fluoroaniline (I-49)

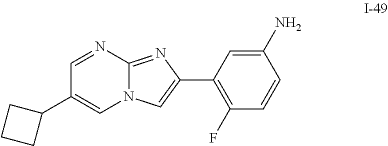 [SMILES: Nc1ccc(F)c(-c2cn3cc(C4CCC4)cnc3n2)c1]

Compound I-49 was prepared following the same synthetic route used to prepared compound I-10 using 5-cyclobutylpyrimidin-2-amine as starting material.

### Example 35

### Synthesis of tert-butyl (2-(5-amino-2-fluorophenyl)imidazo[1,2-a]pyrimidin-6-yl)(methyl)carbamate (I-50)

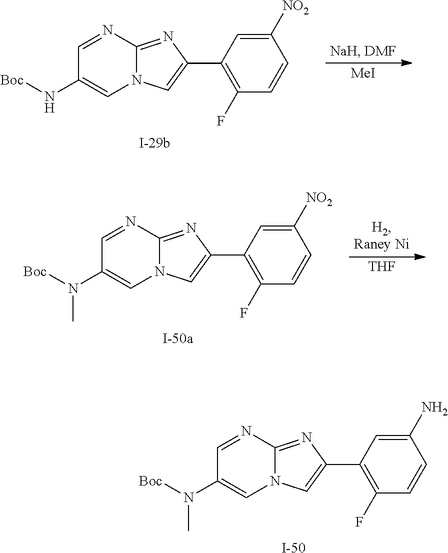

### Synthesis of compound I-50a

To the solution of compound I-29b (1 g, 2.68 mmol) in DMF (20 mL) was added NaH (128 mg, 3.2 mmol). The mixture was stirred for 10 min at 0° C., then Mel (74 mg, 5.2 mmol) was added in one portion and the mixture was stirred for 3 h until the reaction was complete. The resulting mixture was quenched with saturated NH₄Cl solution (10 mL). The mixture was extracted with Ethyl Acetate (100 mL×2), the combined organics were washed with saturated NaCl solution (50 mL), and the organic layer was dried over Na₂SO₄, filtered, and concentrated in vacuo. The crude material was triturated with CH₃OH (100 mL) and the solids were filtered and dried in vacuo to give compound I-50a (150 mg, yield: 37.4%) as a brown solid. ¹H NMR (400 MHz, DMSO-d₆) δ 9.08 (d, J=2.51 Hz, 1H), 9.04 (dd, J=6.27, 3.01 Hz, 1H), 8.67 (d, J=2.51 Hz, 1H), 8.38 (d, J=4.27 Hz, 1H), 8.29 (dt, J=8.72, 3.67 Hz, 1H), 7.67 (t, J=9.79 Hz, 1H), 3.26 (s, 3H), 1.42 (s, 9H)

### Synthesis of compound I-50

To the solution of compound I-50a (460 mg, 1.18 mmol) in THF (40 mL) was added Raney Ni (500 mg). The reaction mixture was stirred at room temperature for 2 hours under H₂ atmosphere, the mixture was filtered through a celite pad and the filtrate was concentrated in vacuo. The crude material was triturated with MeOH (10 mL×2) and the solids were filtered, dried in vacuo to give compound I-50 (380 mg, yield: 91%) as a brown solid. MS m/z=358.1 (M+1).

### Example 36

### Synthesis of 4-fluoro-3-(6-phenylimidazo[1,2-a]pyrimidin-2-yl)aniline (I-51)

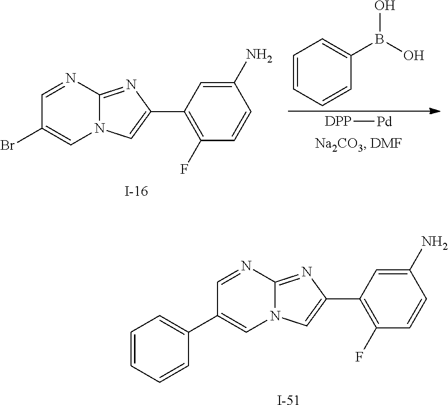

In a vial, I-16 (75 mg, 0.244 mmol), phenylboronic acid (32.8 mg, 0.269 mmol), sodium carbonate (78 mg, 0.733 mmol), and SiliaCat Dpp-Pd (48.8 mg, 0.012 mmol) were taken up in N,N-Dimethylformamide (4 mL), and the resulting suspension was sparged with Ar, and subsequently heated to 75° C. overnight. The reaction was filtered and purified by mass-triggered preparatory HPLC Fractions were concentrated by Genevac, residue taken up in Ethyl Acetate, neutralized (washed with 10% NaHCO₃), dried over MgSO₄, and concentrated in vacuo to give the product I-51 (30.7 mg, 0.101 mmol, 41.3% yield) as a light yellow solid. ¹H NMR (400 MHz, DMSO-d₆) δ 9.33 (d, J=2.6 Hz, 1H), 8.93 (d, J=2.6 Hz, 1H), 8.20 (d, J=4.2 Hz, 1H), 7.83-7.74 (m, 2H), 7.60-7.51 (m, 3H), 7.50-7.41 (m, 1H), 7.00 (dd, J=11.3, 8.7 Hz, 1H), 6.55 (ddd, J=8.8, 4.3, 2.8 Hz, 1H), 5.15 (s, 2H). MS m/z=305.1 (M+1).

### Example 37

### Synthesis of 5-(prop-1-en-2-yl)pyrimidin-2-amine (1-52)

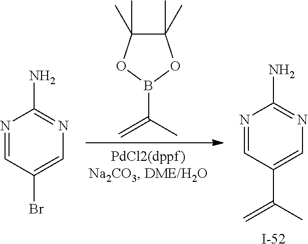

2-Amino-5-bromopyrimidine (2.00 g, 11.49 mmol), isopropenylboronic acid pinacol ester (2.125 g, 12.64 mmol), sodium carbonate (4.87 g, 46.0 mmol) and PdCl₂(dppf) (0.469 g, 0.575 mmol) were taken up in DME (60 mL) and water (20.00 mL) and the resulting solution was sparged with Ar and heated to 75° C. overnight. The crude material was evaporated on silica gel and purified by flash column chromatography to give the product I-52 (2.5095 g, 18.57 mmol, 162% yield) as a tan solid. MS m/z=136.1 (M+1).

### Example 38

### Synthesis of 4-chloro-3-(6-chloroimidazo[1,2-a]pyrimidin-2-yl)aniline (I-53)

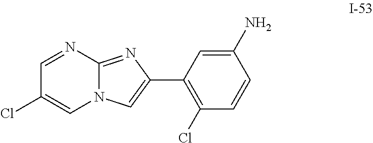 [SMILES: Nc1ccc(Cl)c(-c2cn3cc(Cl)cnc3n2)c1]

Compound I-53 was prepared following the same synthetic route used to prepared compound I-4 using 5-chloropyrimidin-2-amine as starting material.

### Example 39

### Synthesis of 4-iodo-1-isopropyl-1H-imidazole (I-54)

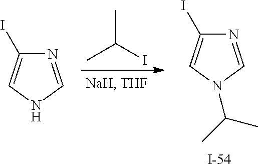

To a solution of 4-iodo-1H-imidazole (1.0 g, 5.2 mmol) in THF (15 mL) was added NaH (250 mg, 6.2 mmol) at 0° C. under N₂ atmosphere. This mixture was stirred at 0° C. for 1 hour, then 2-iodopropane was added. This mixture was stirred at room temperature for 3 hours, then water (15 mL) was added at 0° C. and the mixture was extracted with Ethyl Acetate (15 mL). The organic layer was concentrated and purified by column chromatography (PE:EA=3:1) to give compound I-54 (300 mg, yield, 22%). ¹H NMR (400 MHz, CDCl₃) δ 7.51 (s, 1H), 7.06 (d, J=1.38 Hz, 1H), 4.23-4.39 (M, 1H), 1.50 (d, J=6.65 Hz, 6H).

### Example 40

### Synthesis of benzyl (2-(5-amino-2-fluorophenyl)imidazo[1,2-a]pyrimidin-6-yl)carbamate (I-55)

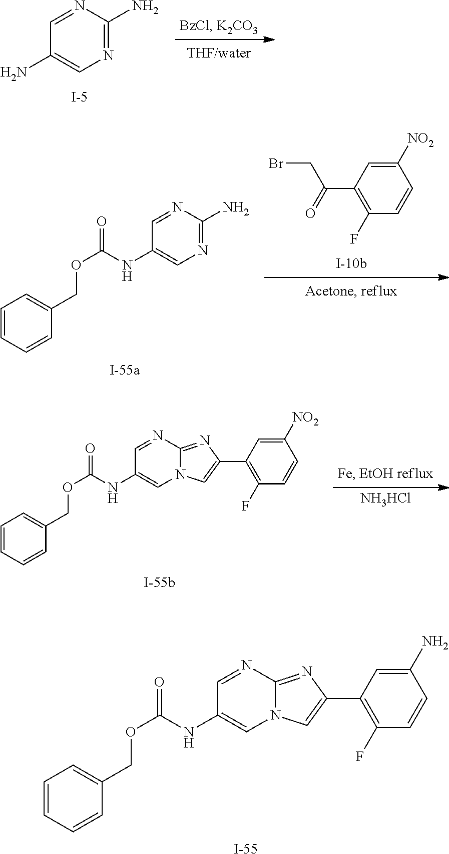

### Synthesis of compound I-55a

In a 150 mL round bottom flask, pyrimidine-2,5-diamine I-5 (3.18 mmol, 500 mg) was dissolved in THF/water (1:1) and potassium carbonate (4.20 mmol, 580 mg) was added. To this biphasic mixture was slowly added benzyl chloroformate (3.45 mmol, 0.490 mL) over 10 minutes. The reaction mixture was then stirred at RT for 2 hours. The mixture was diluted with Ethyl Acetate. The two layers were separated, and the aqueous layer was extracted with Ethyl Acetate (2×25 mL). The combined organic layer was washed with brine (1×30 mL), dried over Na₂SO₄, filtered and evaporated under reduced pressure to give the crude product which was purified by column chromatography (Ethyl acetate/cyclohexane) to give compound I-55a as a pale oil (514 mg, 33%, purity 50% by LCMS).

### Synthesis of compound I-55b

In a 100 mL round bottom flask containing benzyl (2-aminopyrimidin-5-yl)carbamate I-55a [symbol] nitrophenyl)ethanone I-10b (1.044 mmol, 274 mg) was added. The reaction mixture was stirred at reflux (60-65° C.) for 14 hours. The cloudy solution was filtered over Buchner and the beige solid recovered was adsorbed onto silica. Purification of the material by flash column chromatography (Ethyl acetate/cyclohexane) afforded the desired product as a light yellow solid (100 mg, 23%). ¹H NMR (400 MHz, DMSO-d₆) δ 10.26 (brs, 1H), 9.34 (brs, 1H), 9.03 (dd, J=6.40, 3.01 Hz, 1H), 8.47-8.62 (m, 2H), 8.17-8.35 (m, 1H), 7.66 (dd, J=10.29, 9.16 Hz, 1H), 7.30-7.52 (m, 5H), 5.22 (s, 2H). MS m/z=407.7 (M+1).

### Synthesis of compound I-55

In a 100 mL round bottom flask containing benzyl (2-(2-fluoro-5-nitrophenyl)imidazo[1,2-a]pyrimidin-6-yl)carbamate I-55b (0.405 mmol, 165 mg) was added 15 mL of EtOH and 5 mL of water. Iron powder (2.025 mmol, 113 mg)) followed by ammonia hydrochloride (0.405 mmol, 23 mg) were then added and the reaction mixture was stirred at reflux (85° C.) for 1 h 30 minutes. After filtration over celite, the filtrate was concentrated in vacuo. The material obtained was partioned between water and Ethyl Acetate. The organic layer was recovered, dried over Na₂SO₄, filtered and evaporated under reduced pressure to give the crude product as a pale yellow solid (105 mg, 67%). ¹H NMR (400 MHz, DMSO-d₆) δ 9.26 (brs, 1H), 8.46 (d, J=2.69 Hz, 1H), 8.27 (d, J=4.16 Hz, 1H), 7.32-7.53 (m, 7H), 6.96 (dd, J=11.19, 8.74 Hz, 1H), 6.46-6.59 (m, 1H), 5.21 (s, 2H), 5.08 (s, 2H). MS m/z=377.7 (M+1).

### Example 41

### Synthesis of benzyl (2-(3-amino-4-fluorophenyl)imidazo[1,2-a]pyrimidin-6-yl)carbamate (I-56)

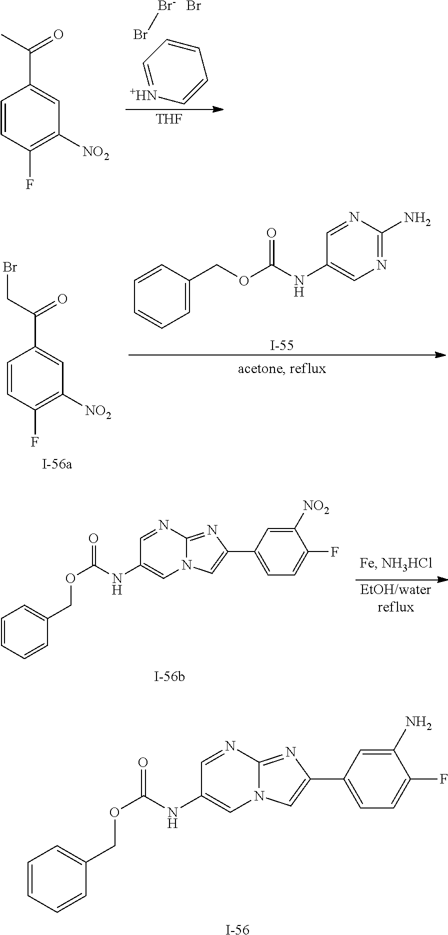

### Synthesis of compound I-56a

In a 100 mL round bottom flask, 1-(4-fluoro-3-nitrophenyl)ethanone (8.19 mmol, 1.5 g) was dissolved in THF and pyridine hydrobromide perbromide (8.60 mmol, 2.75 g) was added portion wise over 10 minutes. Upon addition a cloudy solution was obtained and was stirred at room temperature for 4 hours. The reaction mixture was filtered over Büchner. The white solid obtained was washed with Ethyl Acetate and the filtrate was concentrated in vacuo and adsorbed onto silica gel. Purification by flash column chromatography yielded the desired product I-56a as an orange oil (1.15 g, 50%). ¹H NMR (400 MHz, CDCl₃) δ 8.71 (dd, J=6.97, 2.20 Hz, 1H), 8.30 (ddd, J=8.74, 4.10, 2.32 Hz, 1H), 7.46 (dd, J=8.80, 1.10 Hz, 1H), 4.43 (s, 2H)

### Synthesis of compound I-56b

In a 50 mL round bottom flask benzyl (2-aminopyrimidin-5-yl)carbamate I-55 (0.409 mmol, 100 mg) was dissolved in 15 mL of acetone and 2-bromo-1-(4-fluoro-3-nitrophenyl)ethanone I-56a (0.409 mmol, 107 mg) was added. The reaction mixture was stirred at reflux (60-65° C.) for 16 hours. DCM (20 mL) and MeOH (10 mL) were added to the crude mixture and it was adsorbed onto silica. Flash column chromatography (DCM/MeOH) provided the desired compound I-56b as a pale yellow solid (70 mg, 38%). ¹H NMR (400 MHz, DMSO-d₆) δ 9.24 (brs, 1H), 8.65 (dd, J=7.27, 2.26 Hz, 1H), 8.58-8.62 (m, 1H), 8.52 (d, J=2.69 Hz, 1H), 8.3-8.38 (m, 1H), 7.71 (dd, J=11.13, 8.80 Hz, 1H), 7.33-7.49 (m, 6H), 5.21-5.25 (m, 2H). MS m/z=409.0 (M+1).

### Synthesis of compound I-56

In a 100 mL round bottom flask containing benzyl (2-(4-fluoro-3-nitrophenyl) imidazo[1,2-a]pyrimidin-6-yl)carbamate (I-56b) (0.172 mmol, 70 mg) was added 10 mL of EtOH and 2.5 mL of water. Iron powder (0.859 mmol, 48 mg) followed by ammonia hydrochloride (0.172 mmol, 10 mg) was then added and the reaction mixture was stirred at reflux (85° C.) for 1 h 30 minutes. Upon completion, the reaction mixture was filtered over celite and the filtrate was concentrated in vacuo to give the product I-56 (60 mg, 90%). ¹H NMR (400 MHz, DMSO-d₆) δ 9.20 (brs, 1H), 8.42 (d, J=2.69 Hz, 1H), 8.22-8.29 (m, 1H), 7.33-7.50 (m, 7H), 7.00-7.10 (m, 2H), 5.24 (s, 2H), 5.21 (s, 2H). MS m/z=378.9 (M+1).

### Example 42

### Synthesis of N-(4-fluoro-3-(6-hydroxyimidazo[1,2-a]pyrimidin-2-yl)phenyl)-2,4-dimethyloxazole-5-carboxamide (I-57)

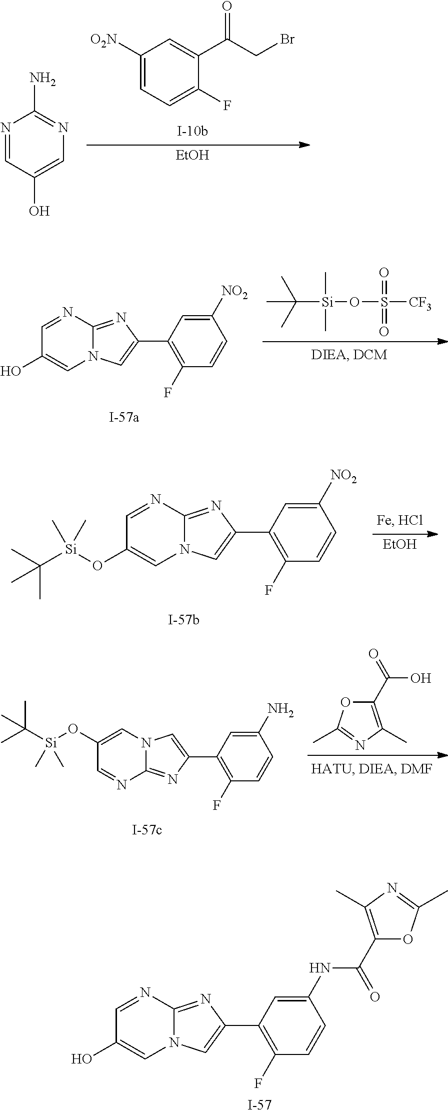

### Synthesis of I-57a

2-aminopyrimidin-5-ol (0.42 g, 3.82 mmol) and 2-bromo-1-(2-fluoro-5-nitrophenyl)ethanone I-10b (1 g, 3.82 mmol) were combined in EtOH and heated at 120° C. for 10 hours. The resulting mixture was cooled and solvent was evaporated. The solid was redissolved in EtOAc and saturated NaHCO₃. The organic phase was isolated and evaporated to yield crude 2-(2-fluoro-5-nitrophenyl)imidazo[1,2-a]pyrimidin-6-ol I-57a.

### Synthesis of I-57b

The crude product was then dissolved in dry CH₂Cl₂ and iPr₂NEt (7.41 g) was added. TBSOTf (6 g) was added dropwise into the reaction solution at 0° C. After the addition, the reaction was warmed to room temperature. After 30 minute, saturated NH₄Cl was added to the mixture and the phases were separated. The organic phase was dried over Na₂SO₄, filtered and evaporated. The crude mixture was purified over silica gel using EtOAc and Hexane) to give 6-((tert-butyldimethylsilypoxy)-2-(2-fluoro-5-nitrophenyl)imidazo[1,2-a]pyrimidine I-57b (0.5 g, 33%).

### Synthesis of I-57c

Two drops of concentrated HCl were added to a stirred and heated (55° C.) suspension of iron powder (10 g) in EtOH (200 mL). After 20 minutes, 6-((tert-butyldimethylsilyl)oxy)-2-(2-fluoro-5-nitrophenyl)imidazo[1,2-a]pyrimidine (I-57b) was added into the reaction mixture followed by saturated NH₄Cl (100 mL). The resulting mixture was stirred at 60° C. for 3 hours before it was filtered and the solvent evaporated. The solid was redissolved in EtOAc and saturated NaHCO₃, the organic phase was dried with Na₂SO₄, and the crude product was purified over silica gel using CH₂Cl₂ and MeOH to give 3-(6-((tert-butyldimethylsilyl)oxy)imidazo[1,2-a]pyrimidin-2-yl)-4-fluoroaniline I-57c.

### Synthesis of 1-57

HATU (1.17 g, 3 mmol) was added to a stirred solution of 2,4-dimethyloxazole-5-carboxylic acid (0.39 g, 2.82 mmol) and DIEA (1.65 g, 12.8 mmol) in dry DMF (10 mL). After 10 minutes, 3-(6-((tert-butyldimethylsilyl)oxy)imidazo[1,2-a]pyrimidin-2-yl)-4-fluoroaniline I-57c (0.92 g, 2.57 mmol) was added to the solution. After 3 hours, the solvent was evaporated and the resulting residue was dissolved in EtOAc and 3 mL of 1M TBAF in THF was added and stirred for 20 minutes. The resulting mixture was poured into saturated NH₄Cl. The organic phase was isolated and purified by column chromatography using CH₂Cl₂ and MeOH to give N-(4-fluoro-3-(6-hydroxyimidazo[1,2-a]pyrimidin-2-yl)phenyl)-2,4-dimethyloxazole-5-carboxamide I-57 as a solid. ¹H NMR (400 MHz, DMSO-d₆) δ 8.72 (dd, J=7.0, 2.8 Hz, 1H), 8.53 (d, J=2.9 Hz, 1H), 8.41 (d, J=2.9 Hz, 1H), 8.26 (d, J=4.3 Hz, 1H), 7.81 (ddd, J=8.9, 4.5, 2.8 Hz, 1H), 7.34 (dd, J=11.1, 9.0 Hz, 1H), 2.48 (s, 3H), 2.25 (s, 3H). MS m/z=368.1 (M+1).

### Example 43

### Synthesis of N-(4-fluoro-3-(6-hydroxyimidazo[1,2-a]pyrimidin-2-yl)phenyl)pyrrolidine-1-carboxamide (I-58)

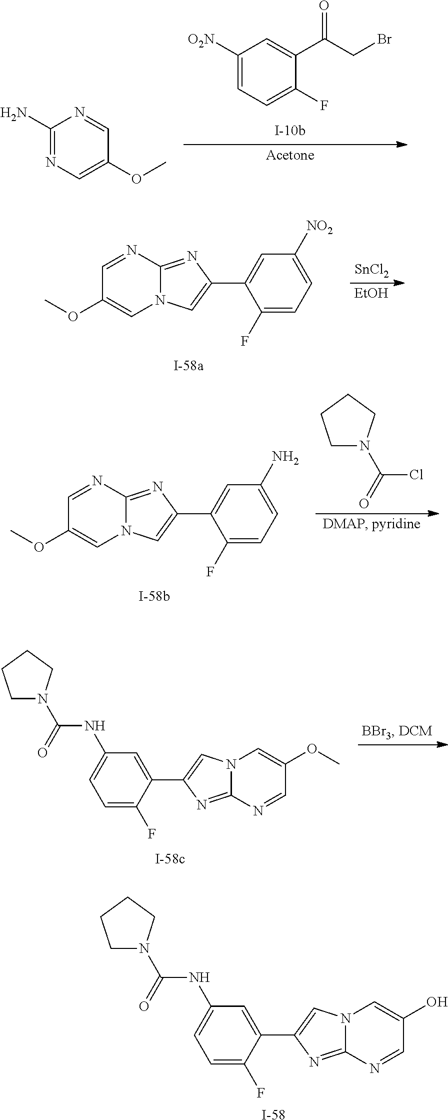

### Synthesis of I-58a

In a flask, 2-bromo-1-(2-fluoro-5-nitrophenyl)ethanone (1.00 g, 3.82 mmol) and 2-amino-5-methoxypyrimidine (0.478 g, 3.82 mmol) were taken up in Acetone (38.2 mL) and heated to reflux overnight. The reaction was cooled, filtered, and the solids were washed with acetone (×2) to give the product I-58a (764.8 mg, 2.65 mmol, 69.5% yield) as a tan solid.

### Synthesis of I-58b

In a vial, intermediate I-58a (765 mg, 2.65 mmol) and tin(II) chloride (2396 mg, 10.62 mmol) were taken up in ethanol (10 mL) and heated to reflux overnight. The reaction was quenched with NaOH at reflux. The reaction was then poured into H₂O and extracted with EtOAc (×3). The organics were then washed with brine, dried over MgSO₄, and concentrated in vacuo to give the product I-58b (240.7 mg, 0.932 mmol, 35.1% yield) as a yellow solid.

### Synthesis of I-58c

In a vial, intermediate I-58b (241 mg, 0.933 mmol) was taken up in pyridine (10 mL). To the resulting solution was added pyrrolidine carbonyl chloride (0.155 mL, 1.400 mmol) and DMAP (11.40 mg, 0.093 mmol). The mixture was stirred overnight. The remaining crude material was evaporated on silica gel and purified by flash column chromatography to give the product I-58c (153.6 mg, 0.419 mmol, 44.9% yield) as a yellow solid.

### Synthesis of I-58

In a vial, I-58c (150.2 mg, 0.423 mmol) was taken up in dichloromethane (5 mL), cooled to −78° C., then to the resulting solution was added boron tribromide (1.691 mL, 1.691 mmol), and the reaction was allowed to slowly warm to room temperature and was stirred overnight. A small amount of product was observed by LCMS. Two additional portions of boron tribromide (1.691 mL, 1.691 mmol) were added to the reaction and the mixture was stirred for 5 days. The resulting material was evaporated on silica gel and purified by flash column chromatography to give the product I-58 (66.3 mg, 0.194 mmol, 46.0% yield) as a yellow oil.

### Example 44

### Synthesis of N-(3-(6-aminoimidazo[1,2-a]pyrimidin-2-yl)-4-fluorophenyl)azetidine-1-carboxamide (I-59)

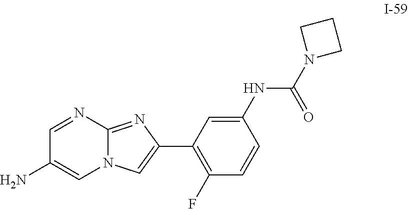 [SMILES: Nc1cnc2nc(-c3cc(NC(=O)N4CCC4)ccc3F)cn2c1]

Compound I-59 was prepared following the protocol described in Example 18 using azetidine hydrochloride. MS m/z=327.1 (M+1).

### Example 45

### Synthesis of 5-fluorofuran-2-carboxylic acid (I-60)

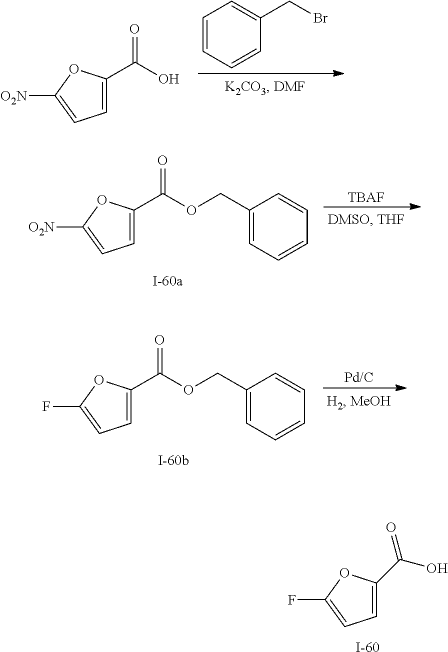

### Synthesis of I-60a

To a solution of 5-nitrofuran-2-carboxylic acid (23 g, 0.15 mol) in DMF (500 mL), K₂CO₃ (30.36 g, 0.22 mol) and benzyl bromide (37.56 g, 0.22 mol) was added. The mixture was stirred at room temperature overnight, diluted with water (1000 mL), extracted with Ethyl Acetate (500 mL×2), dried, concentrated, and purified by column chromatography (20% EA/PE), to give compound I-60a (35 g, yield, 98%) as a yellow solid. ¹H NMR (400 MHz, CDCl₃): 7.39-7.49 (m, 5H), 7.34-7.36 (m, 1H), 7.31-7.33 (m, 1H), 5.42 (s, 2H)

### Synthesis of I-60b

To a solution of compound I-60a (23.5 g, 0.095 mol) in DMSO-d₆ (200 mL), a solution of TBAF (99.42 g, 0.38 mol) in THF (800 mL) was added dropwise. The mixture was stirred at room temperature overnight, diluted with water (1000 mL), extracted with Ethyl Acetate (500 mL×2), dried, concentrated and purified by the column chromatography [symbol] CDCl₃) δ 7.36-7.47 (m, 5H), 7.17 (t, J=3.51 Hz, 1H), 5.65 (dd, J=7.09, 3.58 Hz, 1H), 5.35 (s, 2H).

### Synthesis of I-60

To a solution of compound I-60b (12 g, 0.054 mol) in methanol (120 mL), Pd/C (0.6 g, wet) was added. The mixture was stirred at room temperature under hydrogen. Monitoring by TLC indicated the reaction wasn't completed after 8 hours. The reaction mixture was filtered, concentrated and the residue was washed with petroleum ether to give compound I-60. The solution was concentrated, treated with the same reaction conditions. Monitoring by TLC indicated the reaction was complete after 6 hours. The reaction mixture was filtered and concentrated to give compound I-60 (5.5 g total, yield, 77%) as solid. ¹H NMR (400 MHz, DMSO-d₆) δ 7.28 (t, J=3.58 Hz, 1H), 6.07 (dd, J=7.03, 3.64 Hz, 1H)

### Example 46

### Synthesis of tert-butyl (2-(3-amino-2-fluorophenyl)imidazo[1,2-a]pyrimidin-6-yl)carbamate (I-61)

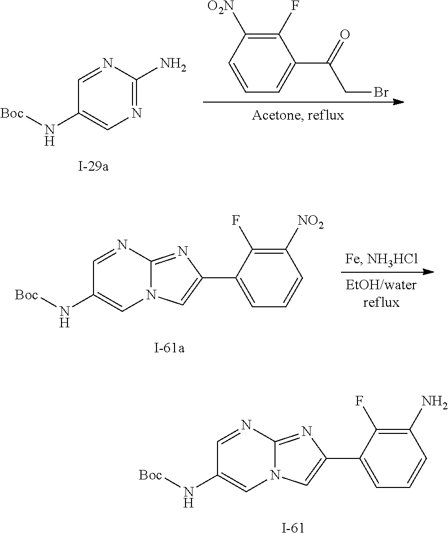

### Synthesis of I-61a

In a 50 mL round bottom flask (tert-butyl (2-aminopyrimidin-5-yl)carbamate I-29a (1.712 mmol, 449 mg) was dissolved in acetone (15 mL) and 2-bromo-1-(2-fluoro-3-nitrophenyl)ethanone (1.712 mmol, 600 mg) was added. The reaction mixture was stirred at reflux (60-65° C.) for 16 hours. The crude mixture was diluted with DCM (20 mL) and MeOH (10 mL) and was adsorbed onto silica gel. Flash column chromatography (Cyclohexane/Ethyl Acetate) provided the desired compound I-61a as a pale yellow solid (60 mg, 9%). ¹H NMR (400 MHz, DMSO-d₆) δ 9.84 (brs, 1H), 9.35 (brs, 1H), 8.60 (td, J=7.27, 1.83 Hz, 1H), 8.48-8.53 (m, 2H), 8.08-8.14 (m, 1H), 7.56 (t, J=8.07 Hz, 1H), 1.50-1.54 (m, 9H).

### Synthesis of I-61

In a 100 mL round bottom flask, tert-butyl (2-(2-fluoro-3-nitrophenyl)imidazo[1,2-a]pyrimidin-6-yl)carbamate I-60a (0.214 mmol, 80 mg) was added EtOH (10 mL) and 2.5 mL of water. Iron powder (0.859 mmol, 48 mg) and ammonia hydrochloride (0.257 mmol, 14 mg) were then added and the reaction mixture was stirred at reflux (85° C.) for 1 h 30 minutes. Upon completion, the reaction mixture was filtered over celite and the filtrate was concentrated in vacuo to give the product I-61 (35 mg, 38%).

### Example 47

### Synthesis of 3-(6-cyclopropylimidazo[1,2-a]pyrimidin-2-yl)-4-fluoroaniline(I-62)

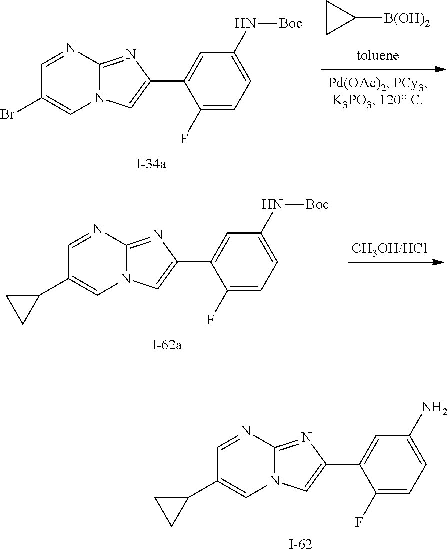

### Synthesis of I-62a

To a solution of compound I-34a (12 g, 0.029 mol) in toluene (300 mL) were added cyclopropylboronic acid (20 g, 0.235 mol), K₃PO₃ (18.5 g, 0.118 mol), PCy₃ (1.624 g, 0.0058 mol), H₂O (30 mL) and Pd(OAc)₂ (0.264 g, 1.1 mmol). The reaction mixture was stirred at 120° C. for 10 hours under N₂ atmosphere. After filtration, the resulting solid was dissolved in THF:EA=1:1(500 mL) and washed with a solution of NaHCO₃ (200 mL×2) and brine(100 mL). The organic layer was filtered through a pad of silica gel and the pad was washed with THF (100 mL×2). The layer was concentrated and the residue was triturated with MeOH (40 mL) and filtered to give product compound I-62a (8 g, yield: 74%) as a white solid. ¹H NMR (400 MHz, DMSO-d₆) δ 9.50 (brs, 1H), 8.76 (d, J=2.26 Hz, 1H), 8.42-8.51 (m, 2H), 8.13 (d, J=4.27 Hz, 1H), 7.39 (m, 1H), 7.18-7.26 (m, 1H), 2.02 (brs, 1H), 1.48 (s, 9H), 1.01 (dd, J=8.28, 1.51 Hz, 2H), 0.79 (d, J=5.77 Hz, 2H).

### Synthesis of I-62

A solution of compound I-62a (8 g, crude) in CH₃OH/HCl (50 mL) was stirred at room temperature for 8 hours. The mixture was filtered and the filter cake was poured into the solution of NaHCO₃(100 mL) and stirred for 1 hour. The aqueous layer was extracted with Ethyl Acetate/THF=1:1(100 mL×3). The combined organic layers were washed with an aqueous solution of NaCl (50 mL) and dried over Na₂SO₄ and concentrated. The solid was triturated with MeOH (10 mL) to give product I-62 (3.5 g, yield: 60%) as a white solid. ¹H NMR (400 MHz, DMSO-d₆) δ 8.75 (d, J=2.26 Hz, 1H), 8.44 (d, J=2.51 Hz, 1H), 8.06 (d, J=4.02 Hz, 1H), 7.50 (dd, J=6.02, 2.51 Hz, 1H), 6.97 (dd, J=11.04, 8.78 Hz, 1H), 6.48-6.61 (m, 1H), 5.11 (brs, 2H), 1.92-2.08 (m, 1H), 0.94-1.06 (m, 2H), 0.69-0.86 (m, 2H). MS m/z=268 (M+1).

### Example 48

### Synthesis of 3-(3-(6-aminoimidazo[1,2-a]pyrimidin-2-yl)-4-fluorophenyl)-1,1-dimethylurea (I-63)

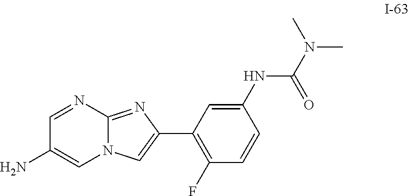 [SMILES: CN(C)C(=O)Nc1ccc(F)c(-c2cn3cc(N)cnc3n2)c1]

Compound I-63 was prepared following the protocol described in Example 18 using dimethylamine hydrochloride. MS m/z=327.1 (M+1).

### Example 49

### Synthesis of N-(3-(6-aminoimidazo[1,2-a]pyrimidin-2-yl)-4-fluorophenyl)azetidine-1-carboxamide (I-64)

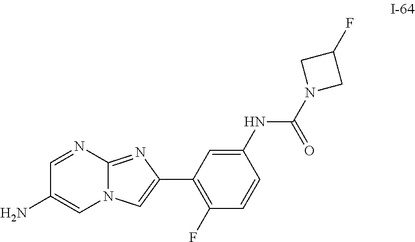 [SMILES: Nc1cnc2nc(-c3cc(NC(=O)N4CC(F)C4)ccc3F)cn2c1]

Compound I-64 was prepared following the protocol described in Example 18 using 3-fluoroazetidine hydrochloride.

### Example 50

### Synthesis of (2-(5-((tert-butoxycarbonyl)amino)-2-fluorophenyl)imidazo[1,2-a]pyrimidin-6-yl)boronic acid (I-65)

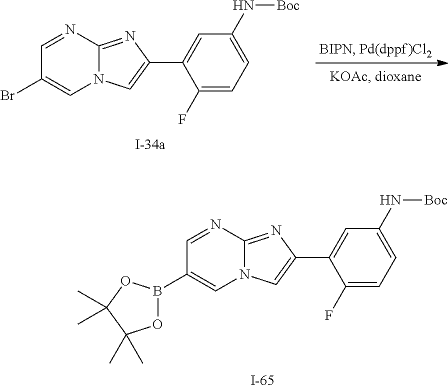

A mixture of compound I-34a (5 g, 12.28 mmol), BIPN (12 g, 49 mmol), anhydrous KOAc (3.6 g, 36.8 mmol) and Pd(dppf)Cl₂ (420 mg, 0.6 mmol) in anhydrous 1,4-dioxane (150 mL) was stirred at 120° C. under N₂ overnight. The reaction mixture was concentrated, diluted with THF (400 mL), passed through a short silica gel column, and the column was washed with THF (100 mL×2). The THF was removed under vacuum and the residue was triturated with t-BuOMe (100 mL) to give compound I-65 (3 g, yield: 54%) as a brown solid. ¹H NMR (400 MHz, DMSO-d₆) δ 9.53 (brs, 1H), 9.28 (d, J=1.51 Hz, 1H), 8.61 (s, 1H), 8.46 (d, J=4.27 Hz, 1H), 8.31 (d, J=4.27 Hz, 1H), 7.42 (d, J=3.51 Hz, 1H), 7.15-7.30 (m, 1H), 1.50 (s, 9H), 1.16 (s, 12H).

### Synthesis of Final Compounds

### Example 51

### Synthesis of isopropyl (2-(2-chloro-5-(5-fluorofuran-2-carboxamido)phenyl) imidazo[1,2-a]pyrimidin-6-yl)carbamate (1)

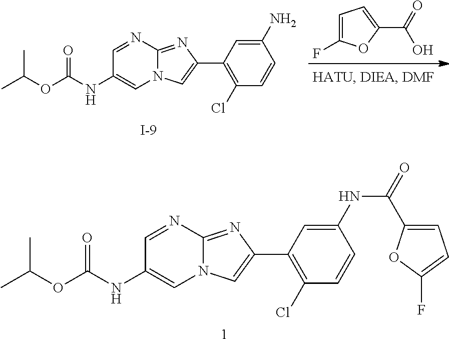

In a 40 mL vial, isopropyl (2-(5-amino-2-chlorophenyl)imidazo[1,2-a]pyrimidin-6-yl)carbamate I-9 (30 mg, 0.09 mmol) was dissolved in DMF (0.3 mL). 5-fluorofuran-2-carboxylic acid (11 mg, 0.09 mmol) was then added to the reaction mixture followed by HATU (65.98 mg, 0.17 mmol) and DIEA (0.043 mL, 0.26 mmol). The reaction was stirred at rt for 8 h. The reaction was monitored was LCMS which indicated the reaction was complete with a new peak of desired mass was observed (M+H=468.1). The reaction mixture was quenched with water and extracted with Ethyl Acetate (3×5 mL). The combined organics were dried over sodium sulfate and concentrated under reduce pressure. The residue was purified by reverse phase HPLC yielding 2-(2-chloro-5-(5-fluorofuran-2-carboxamido)phenyl)imidazo[1,2-a]pyrimidin-6-yl)carbamate 1. The TFA salt was neutralized to a free base using a HCO₃ column. ¹H NMR (400 MHz, DMSO-d₆) δ 10.41 (s, 1H), 10.01 (s, 1H), 9.31 (s, 1H), 8.69 (s, 1H), 8.61 (d, J=2.7 Hz, 1H), 8.51 (d, J=2.7 Hz, 1H), 7.87 (dd, J=2.7 Hz, 8.8, 1H), 7.53 (d, J=8.7 Hz, 1H), 7.47 (t, J=3.6 Hz, 1H), 6.11 (dd, J=3.7 Hz, 7.1, 1H), 5.03-4.85 (m, 1H), 1.29 (d, J=6.2 Hz, 6H). Ms m/z=458.1 (M+1).

### Syntheses of compounds 3, 7, 11, 18, 22 and 41

Compounds 3, 7, 11, 18, 22, 38, and 41 were synthesized by following the amide coupling reaction described above using the appropriate carboxylic acid.

### Example 52

### Synthesis of isopropyl (2-(2-chloro-5-(pyrrolidine-1-carboxamido)phenyl) imidazo[1,2-a]pyrimidin-6-yl)carbamate (2)

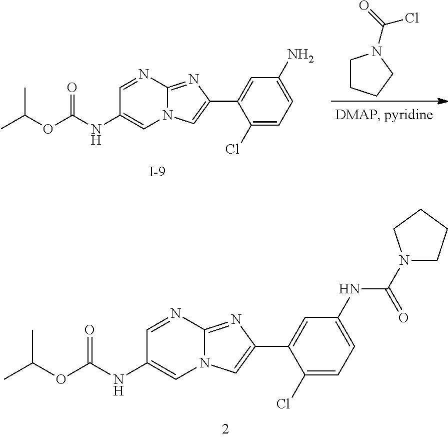

In a 40 mL vial, the intermediate I-9 (100 mg, 0.29 mmol) was dissolved in pyridine (1.0 mL). DMAP (0.71 mg, 0.02 mmol) was then added to the reaction mixture followed by pyrrolidine carbonyl chloride (58 mg, 0.43 mmol). The reaction was stirred at room temperature for 3 days. The reaction was quenched with methanol and evaporated to dryness. The residue was purified by flash chromatography using 60% Ethyl Acetate in Hexane) isocratically, then a gradient to 100% Ethyl Acetate, and kept isocratically to elute product 2. ¹H NMR (400 MHz, DMSO-d₆) δ 9.99 (s, 1H), 9.29 (s, 1H), 8.63 (s, 1H), 8.50 (d, J=2.7 Hz, 1H), 8.44 (s, 1H), 8.39 (d, J=2.7 Hz, 1H), 7.70 (dd, J=2.7, 8.8 Hz, 1H), 7.38 (d, J=8.8 Hz, 1H), 4.99-4.88 (m, 1H), 3.39 (t, J=6.6 Hz, 4H), 1.85 (t, J=6.5 Hz, 4H), 1.29 (d, J=6.2 Hz, 6H). MS m/z=443.1 (M+1).

### Syntheses of compounds 5, 6, 14, 16, 17 and 20

Compounds 5, 6, 14, 16, 17 and 20 were synthesized following the urea coupling reaction described above using intermediate I-8 and the appropriate carbonyl chloride.

### Syntheses of compounds 10, 23 and 40

Compounds 10, 23 and 40 were synthesized using the conditions described above using ethyl and 2-methoxyethyl chlorofomate with I-8 (compound 10 and 23) and isopropyl chloroformate with 1-10 (compound 40).

### Example 53

### Synthesis of isopropyl (2-(2-chloro-5-(3,3-difluoropyrrolidine-1-carboxamido)phenyl) imidazo[1,2-a]pyrimidin-6-yl)carbamate (4)

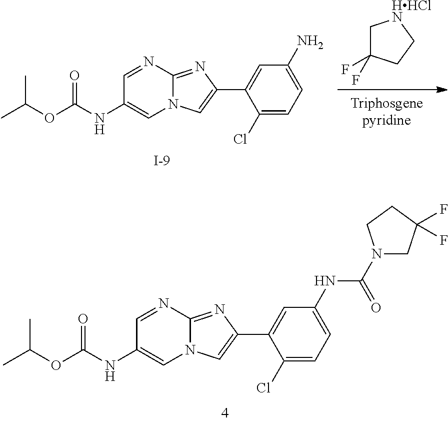

To a solution of difluoropyrrolidine. HCl (1 eq.) in anhydrous pyridine (0.3 mM) was added triphosgene (1.2 eq.) at 0° C. and the mixture was stirred for 30 minutes. The bright orange reaction mixture with yellow precipitate was then added directly to a solution of intermediate I-9 in anhydrous pyridine (0.3 mM). DMAP (0.02 eq.) was added and the reaction was stirred overnight. The reaction mixture was concentrated and purified by mass-triggered reverse phase column chromatography preparative system to give compound 4 ¹H NMR (400 MHz, DMSO-d₆) δ 10.00 (s, 1H), 9.29 (s, 1H), 8.67 (d, J=19.1 Hz, 2H), 8.50 (d, J=2.3 Hz, 1H), 8.39 (d, J=2.1 Hz, 1H), 7.69 (d, J=8.8 Hz, 1H), 7.41 (d, J=8.8 Hz, 1H), 4.94 (dt, J=5.9, 11.8 Hz, 1H), 3.85 (t, J=13.2 Hz, 2H), 3.65 (t, J=7.2 Hz, 4H), 1.29 (d, J=6.2 Hz, 6H); ¹⁹FNMR (375 MHz, DMSO-d₆): 100.44. MS m/z=479 (M+1).

### Example 54

### Synthesis of isopropyl (2-(2-fluoro-5-(furan-2-carboxamido)phenyl) imidazo[1,2-a]pyrimidin-6-yl)carbamate (12)

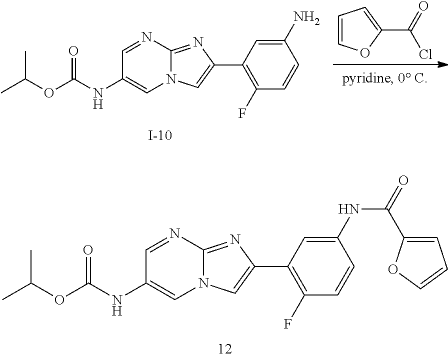

In a 40 mL vial, pyridine (10 mL) was added to the intermediate I-10 (0.5 g, 1.518 mmol) to give a yellow solution. To this solution was added furan-2-carbonyl chloride (0.198 g, 1.518 mmol) at 0° C. and the resulting mixture was stirred for 1 hour. The reaction mixture [symbol] repeated once again to get rid off any extra pyridine. All organic phases were combined, dried over sodium sulfate and purified via flash chromatography to give product 12 (Ethyl Acetate/MeOH=0-10%). ¹H NMR (400 MHz, DMSO-d₆) δ 10.43 (s, 1H), 10.06 (s, 1H), 9.36 (s, 1H), 8.69 (dd, J=2.8, 6.9 Hz, 1H), 8.56 (d, J=2.7 Hz, 1H), 8.45 (d, J=4.2 Hz, 1H), 8.02 (d, J=1.0 Hz, 1H), 7.95-7.85 (m, 1H), 7.46 (d, J=3.4 Hz, 1H), 7.37 (dd, J=9.0, 10.9 Hz, 1H), 6.78 (dd, J=1.7, 3.5 Hz, 1H), 5.00 (dt, J=6.3, 12.5 Hz, 1H), 1.35 (d, J=6.2 Hz, 6H). MS m/z=424 (M+1).

### Syntheses of Compounds 8, 13, and 42

Compounds 8, 13 and 42 were synthesized by following the amide coupling reaction described above and using the intermediate I-8 and the appropriate carbonyl chloride.

### Example 55

### Synthesis of N-(4-chloro-3-(3-fluoro-6-phenylimidazo[1,2-a]pyrimidin-2-yl)phenyl)pyrrolidine-1-carboxamide (15)

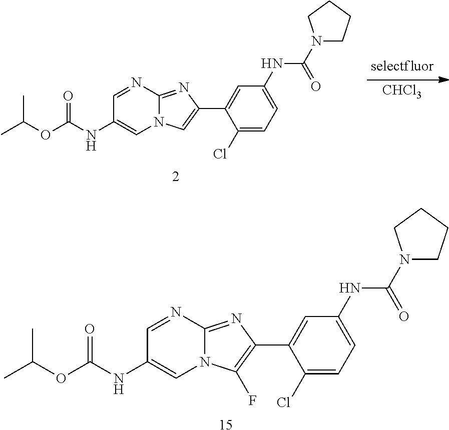

To a solution of isopropyl (2-(2-chloro-5-(pyrrolidine-1-carboxamido)phenyl)imidazo[1,2-a]pyrimidin-6-yl)carbamate 2 (1 eq.) in chloroform (0.3 mM) was added SelectFluor (1.5 eq.) and the reaction was allowed to stir in a sealed tube at 90° C. An additional 1.5 equivalent of SelectFluor was added each day for two days, and the reaction was stirred and monitored over 1 week. At completion, the reaction mixture was concentrated in vacuo and the residue was redissolved in methanol. Purification of the compound was done via mass-triggered reverse phase column chromatography preparative system leading to isopropyl (2-(2-chloro-5-(pyrrolidine-1-carboxamido)phenyl)-3-fluoroimidazo[1,2-a]pyrimidin-6-yl)carbamate 15 (25% yield). ¹H NMR (600 MHz, DMSO-d₆) δ 9.14 (d, J=2.2 Hz, 1H), 8.98 (d, J=2.5 Hz, 1H), 8.45 (s, 1H), 8.00 (d, J=2.7 Hz, 1H), 7.87 (m, 2H), 7.74 (dd, J=2.7, 8.8 Hz, 1H), 7.55 (m, 2H), 7.47 (m, 2H), 3.38 (t, J=6.7 Hz, 4H), 1.85 (d, J=6.6 Hz, 4H). ¹⁹F NMR (375 MHz, DMSO-d₆) δ−146.18. MS m/z=461 (M+1).

### Synthesis of Compound 39

Compound 39 was synthesized in a similar fashion by fluorination of compound 29 using SelectFluor.

### Example 56

### Synthesis of isopropyl (2-(2-fluoro-5-nitrophenyl)imidazo[1,2-a]pyrimidin-6-yl)carbamate (19)

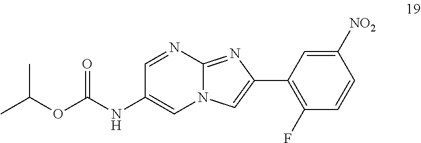 [SMILES: CC(C)OC(=O)Nc1cnc2nc(-c3cc([N+](=O)[O-])ccc3F)cn2c1]

Compound 19 was obtained as an intermediate in the synthesis of I-10.

### Example 57

### Synthesis of N-(4-chloro-3-(6-(4-(methylamino)phenyl)imidazo[1,2-a]pyrimidin-2-yl)phenyl)pyrrolidine-1-carboxamide (26)

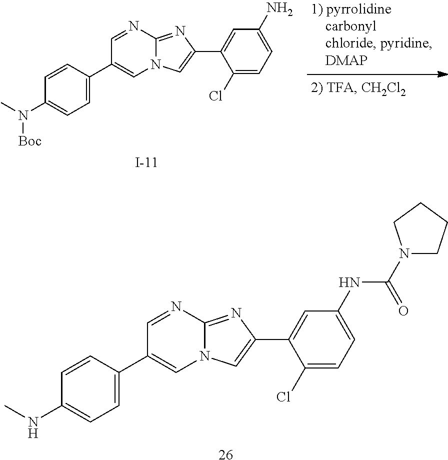

tert-Butyl-(4-(2-(5-amino-2-chlorophenyl)imidazo[1,2-a]pyrimidin-6-yl)phenyl)(methyl)carbamate I-11 was coupled with pyrrolidine-1-carbonyl chloride using a protocol similar to the one used in the synthesis of compound 2. Purification of the compound was carried out using mass-triggered reverse phase column chromatography preparative system. MS m/z=548 (M+1).

Tert-butyl(4-(2-(2-chloro-5-(pyrrolidine-1-carboxamido)phenyl)imidazo[1,2-a]pyrimidin-6-yl)phenyl)(methyl)carbamate was treated with 5% TFA in DCM and was stirred overnight at room temperature. At completion, the reaction mixture was concentrated in vacuo and the residue was redissolved in methanol. Purification of compound 26 was done via mass-triggered reverse phase column chromatography preparative system. ¹H NMR (600 MHz, DMSO-d₆) δ 9.17 (d, J=2.6 Hz, 1H), 8.88 (d, J=2.6 Hz, 1H), 8.5, 362 (s, 1H), 8.46 (m, 2H), 7.73 (m, 1H), 7.52 (d, J=8.6 Hz, 2H), 7.40 (d, J=8.8 Hz, 1H), 6.68 (d, J=8.7 Hz, 2H), 5.97 (s, 1H), 3.40 (s, 4H), 2.73 (d, J=5.0 Hz, 3H), 1.86 (s, 4H). MS m/z=447.2 (M+1).

### Syntheses of Compounds 21, 36 and 37

Compounds 21, 36 and 37 were synthesized according to the procedure described above using I-11 and 2-methoxyethyl chloroformate and ethyl chloroformate.

### Example 58

### Synthesis of methyl (4-(2-(2-chloro-5-(pyrrolidine-1-carboxamido)phenyl)imidazo[1,2-a]pyrimidin-6-yl)phenyl)carbamate (24)

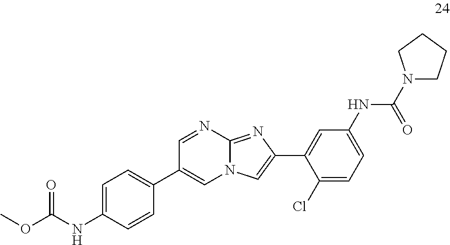 [SMILES: COC(=O)Nc1ccc(-c2cnc3nc(-c4cc(NC(=O)N5CCCC5)ccc4Cl)cn3c2)cc1]

Compound 24 was synthesized using N-(3-(6-bromoimidazo[1,2-a]pyrimidin-2-yl)-4-chlorophenyl)pyrrolidine-1-carboxamide (I-15) as the bromide and (4-((methoxycarbonyl)amino)phenyl)boronic acid using a suzuki coupling protocol as described in the synthesis of I-11. The residue was redissolved in methanol and precipitation yielded compound 24. ¹H NMR (600 MHz, DMSO-d₆) δ 9.86 (s, 1H), 9.30 (d, J=2.6 Hz, 1H), 8.94 (d, J=2.6 Hz, 1H), 8.55 (s, 1H), 8.47 (d, J=2.8 Hz, 2H), 7.73 (m, 3H), 7.62 (d, J=8.5 Hz, 2H), 7.41 (d, J=8.8 Hz, 1H), 3.70 (s, 3H), 3.40 (t, J=6.5 Hz, 4H), 1.86 (t, J=6.4 Hz, 4H). MS m/z=491.2 (M+1).

### Syntheses of Compounds 25, 28 and 29

Compounds 25, 28 and 29 were synthesized according to the protocol described above using 4F-phenyl boronic acid, thiophene-3-boronic acid and phenyl boronic acid respectively.

### Example 59

### Synthesis of methyl (4-(2-(2-chloro-5-(5-methylfuran-2-carboxamido)phenyl)imidazo[1,2-a]pyrimidin-6-yl)phenyl)carbamate (27)

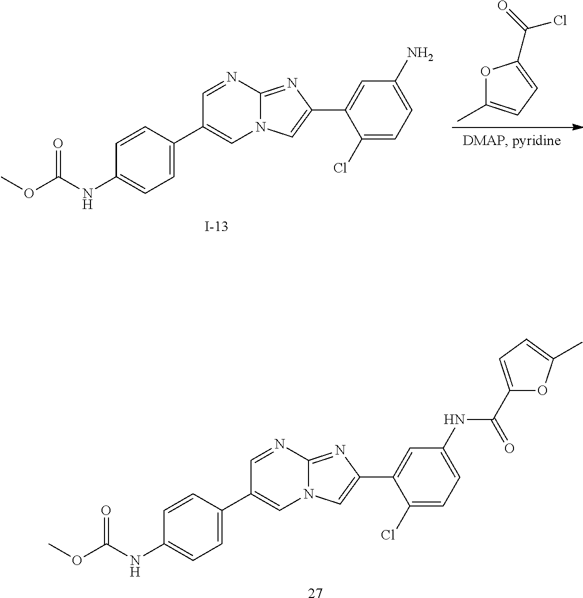

To the 5-methylfuran-2-carbonyl chloride (1.2 eq.) was added a solution of methyl (4-(2-(5-amino-2-chlorophenyl)imidazo[1,2-a]pyrimidin-6-yl)phenyl)carbamate I-13 (1 eq.) in anhydrous pyridine (0.3 mM). The resultant solution was stirred and DMAP (0.02 eq.) was added. The reaction was stirred at room temperature for at least 1 hour to overnight. At completion, the mixture was quenched with methanol, concentrated in vacuo. The residue was purified by HPLC to give product 27. ¹H NMR (600 MHz, DMSO-d₆) δ 10.31 (s, 1H), 9.86 (s, 1H), 9.32 (d, J=2.5 Hz, 1H), 8.97 (d, J=2.5 Hz, 1H), 8.69 (d, J=2.6 Hz, 1H), 8.60 (s, 1H), 7.93 (dd, J=2.7, 8.7 Hz, 1H), 7.73 (d, J=8.6 Hz, 2H), 7.63 (d, J=8.5 Hz, 2H), 7.55 (d, J=8.7 Hz, 1H), 7.33 (d, J=3.3 Hz, 1H), 6.35 (d, J=2.5 Hz, 1H), 3.70 (s, 3H), 2.40 (s, 3H). MS m/z=502.1 (M+1).

### Example 60

### Synthesis of N-(4-chloro-3-(6-phenylimidazo[1,2-a]pyrimidin-2-yl)phenyl)-5-methylfuran-2-carboxamide (31)

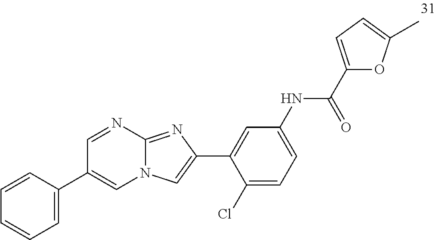 [SMILES: Cc1ccc(C(=O)Nc2ccc(Cl)c(-c3cn4cc(-c5ccccc5)cnc4n3)c2)o1]

Compound 31 was synthesized using N-(3-(6-bromoimidazo[1,2-a]pyrimidin-2-yl)-4-chlorophenyl)-5-methylfuran-2-carboxamide (I-14) as the bromide and phenylboronic acid using the suzuki coupling protocol described in the synthesis of 1-11. The residue was redissolved in methanol and precipitation yielded the compound 31. ¹H NMR (400 MHz, DMSO-d₆) 10.33 (s, 1H), 9.38 (d, J=2.1 Hz, 1H), 8.99 (d, J=2.0 Hz, 1H), 8.71 (d, J=2.5 Hz, 1H), 8.62 (s, 1H), 7.95 (dd, J=2.4, 8.7 Hz, 1H), 7.80 (d, J=7.8 Hz, 2H), 7.52 (dt, J=7.8, 14.7 Hz, 5H), 7.34 (d, J=3.3 Hz, 1H), 6.35 (d, J=3.3 Hz, 1H), 2.40 (s, 3H).

### Synthesis of Compound 30

Compound 30 was synthesized according to the protocol described above using thiophene-3-boronic acid as starting material.

### Example 61

### Synthesis 2-methoxyethyl N-[4-chloro-3-(6-{4-[(methoxycarbonyl)amino]phenyl}imidazo[1,2-a]pyrimidin-2-yl)phenyl]carbamate (32)

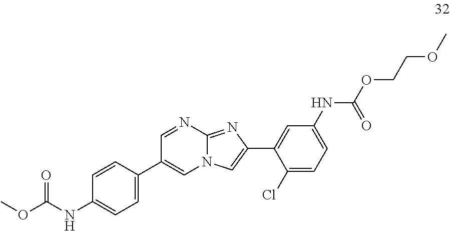

Compound 32 was synthesized using 2-methoxyethyl (3-(6-bromoimidazo[1,2-a]pyrimidin-2-yl)-4-chlorophenyl)carbamate I-12 as the bromide and (4-((methoxycarbonyl)amino)phenyl)boronic acid using the suzuki coupling procedure described in I-13. Purification of the compound was done via mass-triggered reverse phase column chromatography preparative system. ¹H NMR (600 MHz, DMSO-d₆) δ 10.01 (s, 1H), 9.86 (s, 1H), 9.30 (s, 1H), 8.95 (s, 1H), 8.55 (s, 1H), 8.47 (s, 1H), 7.72 (d, J=6.0 Hz, 2H), 7.62 (d, J=6.6 Hz, 2H), 7.50 (dt, J=5.7, 8.5 Hz, 2H), 4.24 (s, 2H), 3.70 (s, 3H), 3.59 (s, 2H), 3.30 (s, 3H). MS m/z=496 (M+1).

### Syntheses of Compounds 34 and 35

Compounds 34 and 35 were synthesized according to the protocol described above using thiophene-3-boronic acid and phenyl boronic acid, respectively.

### Example 62

### Synthesis of N-(4-chloro-3-(6-(4-(methylamino)phenyl)imidazo[1,2-a]pyrimidin-2-yl)phenyl)-5-methylfuran-2-carboxamide (33)

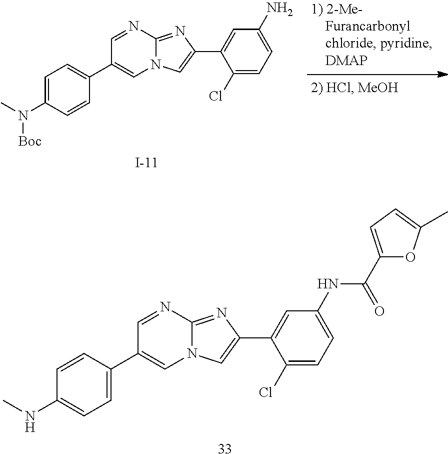

The amide coupling was carried out using the procedure described in the synthesis of 27. The residue was used immediately without further purification. MS m/z=559 (M+1). Tert-Butyl (4-(2-(2-chloro-5-(5-methylfuran-2-carboxamido)phenyl)imidazo[1,2-a]pyrimidin-6-yl)phenyl)(methyl)carbamate was treated with 4.0M HCl in 1,4-dioxane (2 eq.) and was stirred overnight at 50° C. At completion, the reaction mixture was concentrated in vacuo and the residue was redissolved in methanol. Purification of compound 33 was done via mass-triggered reverse phase column chromatography preparative system. ¹H NMR (400 MHz, DMSO-d₆) δ 10.32 (s, 1H), 9.19 (d, J=2.5 Hz, 1H), 8.90 (d, J=2.5 Hz, 1H), 8.69 (d, J=2.6 Hz, 1H), 8.56 (s, 1H), 7.93 (dd, J=2.7, 8.8 Hz, 1H), 7.53 (dd, J=1.7, 8.7 Hz, 3H), 7.34 (d, J=3.3 Hz, 1H), 6.68 (d, J=8.7 Hz, 2H), 6.35 (d, J=3.3 Hz, 1H), 6.00 (q, J=4.9 Hz, 1H), 2.73 (d, J=5.0 Hz, 3H), 2.40 (s, 3H). MS m/z=460 (M+1).

### Example 63

### Syntheses of Compounds 43 to 60

Compounds 43 to 60 were synthesized by following the amide coupling reaction described in Example 51 using the appropriate carboxylic acid.

### Syntheses of Compounds 61 to 120

Compounds 61 to 120 were synthesized by following the amide coupling reaction described above using the intermediate I-10 and the appropriate carboxylic acid.

### Syntheses of Compounds 121 to 147

Compounds 121 to 147 were synthesized by following the amide coupling reaction described above using the intermediate I-33 and the appropriate carboxylic acid.

### Syntheses of Compounds 148 to 185

Compounds 148 to 185 were synthesized by following the amide coupling reaction described above using the intermediate I-31 and the appropriate carboxylic acid. Compounds 179 to 183 used Boc-protected amines that were subsequently cleaved in presence of HCl 4N in 1,4-dioxane. Compounds 184 and 185 were separated by HPLC.

### Syntheses of Compounds 186 to 195

Compounds 186 to 195 were synthesized by following the amide coupling reaction described above using the intermediate I-16 and the appropriate carboxylic acid.

### Example 64

### Synthesis of N-(3-(6-bromoimidazo[1,2-a]pyrimidin-2-yl)-4-fluorophenyl)cyclopentanecarboxamide (196)

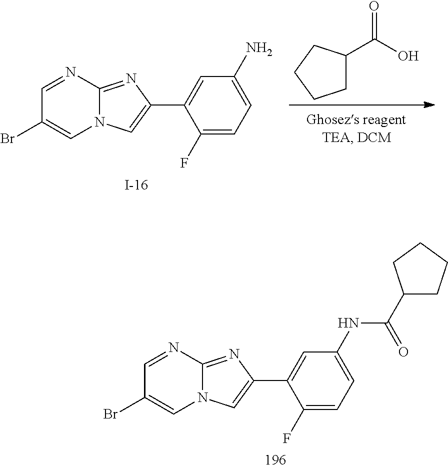

Ghosez's reagent (25.8 μl, 0.195 mmol) was added to a solution of cyclopentanecarboxylic acid (25 μl, 0.231 mmol) in anhydrous dichloromethane (Volume: 814 μl) and the reaction mixture was stirred at room temperature overnight. 3-(6-bromoimidazo[1,2-a]pyrimidin-2-yl)-4-fluoroaniline I-16 (50 mg, 0.163 mmol) and triethylamine (45.4 μl, 0.326 mmol) were solvated in anhydrous dichloromethane (1 mL) and added to the reaction mixture. The reaction mixture was then stirred at room temperature for 2 hours. Saturated sodium bicarbonate was added to the reaction mixture and the organic product was extracted with Ethyl Acetate: DCM (1:10) through a phase separator cartridge. The organic product was concentrated at reduced pressure. Purification was carried out using normal phase column chromatography with solvent system 100% Dichloromethane to 10% methanol in dichloromethane (gradient run over 10 minutes). Fractions containing the product were combined and concentrated at reduced pressure. Further purification was carried out using reverse HPLC to obtain compound 196. ¹H NMR (400 MHz, DMSO-d₆) δ 10.05 (s, 1H), 9.35 (d, J=2.5 Hz, 1H), 8.64 (d, J=2.5 Hz, 1H), 8.51 (dd, J=6.8, 2.8 Hz, 1H), 8.23 (d, J=4.1 Hz, 1H), 7.73 (ddd, J=8.9, 4.6, 2.8 Hz, 1H), 7.28 (dd, J=11.1, 8.9 Hz, 1H), 2.79 (p, J=8.0 Hz, 1H), 1.93-1.80 (m, 2H), 1.81-1.53 (m, 6H).

### Example 65

### Synthesis of Compound 197

Compound 197 was synthesized by following the amide coupling reaction described in Example 54 and using the intermediate I-16, benzoyl chloride, triethylamine as the base (4.4 eq.) and DCM as the solvent.

### Example 66

### Synthesis of Compound 198

Compound 198 was synthesized by following the amide coupling reaction described in Example 51 using the intermediate I-34 and 2-methyloxazole-5-carboxylic acid.

### Syntheses of Compound 199

Compound 199 was synthesized by following the amide coupling reaction described above using the intermediate I-35 and 2,4-dimethyloxazole-5-carboxylic acid.

### Syntheses of Compounds 200 and 201

Compound 200 and 201 were synthesized by following the amide coupling reaction described above using the intermediate I-36 and 2,4-dimethyloxazole-5-carboxylic acid and 2-methyloxazole-5-carboxylic acid respectively.

### Syntheses of Compounds 202 to 204

Compounds 202 to 204 were synthesized by following the amide coupling reaction described above using the intermediate I-8 and the appropriate carboxylic acid.

### Synthesis of Compound 205

Compound 205 was synthesized by following the amide coupling reaction described above using the intermediate I-37 and 2,4-dimethyloxazole-5-carboxylic acid.

### Synthesis of Compound 206

Compound 206 was synthesized by following the amide coupling reaction described above using the intermediate I-39 and 2-methyloxazole-5-carboxylic acid.

### Synthesis of Compound 207

Compound 207 was synthesized by following the amide coupling reaction described above using the intermediate I-46.

### Syntheses of Compounds 208 and 209

Compounds 208 and 209 were synthesized by following the amide coupling reaction described above using the intermediate I-47 and 2,4-dimethyloxazole-5-carboxylic acid and 2-methyloxazole-5-carboxylic acid respectively.

### Syntheses of Compounds 210 and 211

Compounds 210 and 211 were synthesized by following the amide coupling reaction described above using the intermediate I-49 and 2,4-dimethyloxazole-5-carboxylic acid and 2-methyloxazole-5-carboxylic acid respectively.

### Synthesis of Compound 212

Compound 212 was synthesized by following the amide coupling reaction described above using the intermediate I-50 and 2,4-dimethyloxazole-5-carboxylic acid, and removal of the Boc protecting group in presence of HCl in Ethyl Acetate.

### Example 67

### Synthesis of N-(4-chloro-3-(6-phenylimidazo[1,2-a]pyrimidin-2-yl)phenyl)cyclopentanecarboxamide (213)

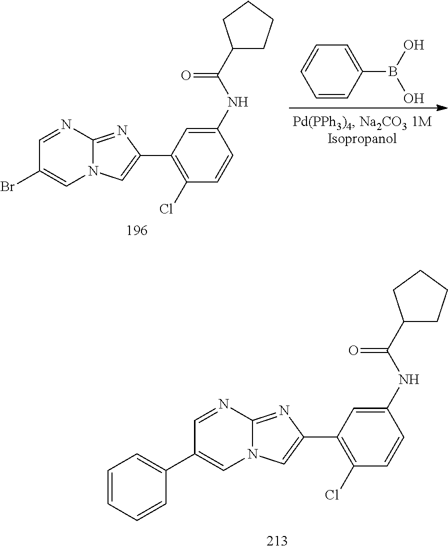 [SMILES: O=C(Nc1ccc(Cl)c(-c2cn3cc(Br)cnc3n2)c1)C1CCCC1 ; OB(O)c1ccccc1 ; "196" ; O=C(Nc1ccc(Cl)c(-c2cn3cc(-c4ccccc4)cnc3n2)c1)C1CCCC1]

Compound 196 (10 mg, 0.025 mmol), phenylboronic acid (3.02 mg, 0.025 mmol) and tetrakis (triphenylphosphine) palladium (1 mg, 0.865 μmol) were weighed into a microwave vial which was evacuated and backfilled with argon before Isopropanol (0.62 mL) and 1M sodium carbonate (0.025 mL) were added. The reaction mixture was subjected to microwave conditions of 120° C. for 20 minutes. The reaction mixture was filtered to remove the excess of catalyst. Purification was carried out using reverse phase HPLC to give product 213 (6.6 mg, 63% yield). ¹H NMR (400 MHz, CD₃OD): 9.13 (d, J=2.5 Hz, 1H), 8.90 (d, J=2.5 Hz, 1H), 8.27 (d, J=3.8 Hz, 1H), 8.12 (dd, J=6.6, 2.7 Hz, 1H), 7.84 (ddd, J=8.9, 4.5, 2.8 Hz, 1H), 7.78-7.67 (m, 2H), 7.60-7.49 (m, 2H), 7.50-7.41 (m, 1H), 7.19 (dd, J=11.0, 8.9 Hz, 1H), 2.83 (q, J=8.0 Hz, 1H), 2.04-1.92 (m, 2H), 1.93-1.73 (m, 5H), 1.67 (dd, J=7.4, 4.8 Hz, 2H).

### Example 68

### Syntheses of Compounds 214 to 218

Compounds 214 to 218 were synthesized following the urea coupling reaction described in Example 52 using the appropriate carbonyl chloride.

### Syntheses of Compounds 219 to 222

Compounds 219 to 222 were synthesized following the urea coupling reaction described above using intermediate I-10 and the appropriate carbonyl chloride.

### Syntheses of Compounds 223 and 224

Compounds 223 and 224 were synthesized following the urea coupling reaction described above using intermediate I-31 and the appropriate carbonyl chloride.

### Syntheses of Compound 225

Compound 225 was synthesized following the urea coupling reaction described above using intermediate I-37 and dimethylcarbamic chloride.

### Example 69 Syntheses of Compound 226 to 229

Compounds 226 to 229 were synthesized following the urea coupling reaction described in Example 53 using intermediate I-10 and the appropriate amines.

### Syntheses of Compound 230 to 234

Compounds 230 to 234 were synthesized following the urea coupling reaction described above using intermediate I-35 and the appropriate amines.

### Syntheses of Compound 235 to 238

Compounds 235 to 238 were synthesized following the urea coupling reaction described above using intermediate I-37 and the appropriate amines.

### Synthesis of Compound 239

Compound 239 was synthesized following the urea coupling reaction described above using intermediate I-47 and dimethylamine hydrochloride.

### Example 70

### Syntheses of Compounds 240 to 242

Compounds 240 to 242 were synthesized by following the amide coupling reaction described in Example 54 using the appropriate carbonyl chloride.

### Syntheses of Compounds 243 to 247

Compounds 243 to 247 were synthesized by following the amide coupling reaction described above and using the intermediate I-8 and the appropriate carbonyl chloride.

### Synthesis of Compound 248

Compound 248 was synthesized by following the amide coupling reaction described above and using the intermediate I-31 and cyclopropanecarbonyl chloride.

### Example 71

### Synthesis of isopropyl (2-(2-chloro-5-(3-isopropyl-3-methylureido) phenyl)imidazo[1,2-a]pyrimidin-6-yl)carbamate (249)

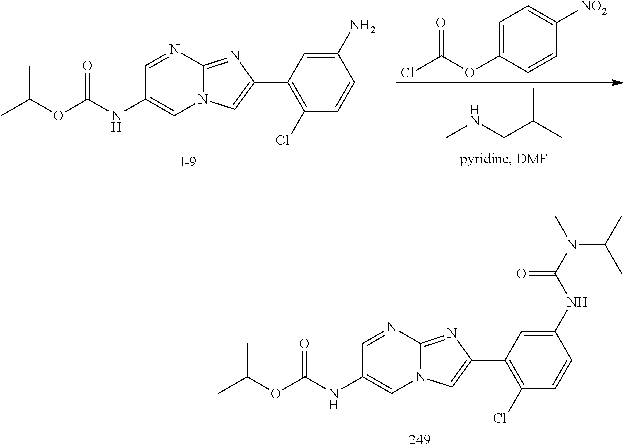

To a stirred solution of isopropyl (2-(5-amino-2-chlorophenyl)imidazo[1,2-a]pyrimidin-6-yl)carbamate (15 mg) in pyridine (1.5 mL) was added 4-nitrophenyl chloroformate (2 eq.). The mixture was stirred for 30 min and N,2-dimethylpropan-1-amine was added in DMF (0.5 mL). The reaction mixture was stirred overnight at 70° C. The resulting mixture was concentrated in vacuo and purified by HPLC to give product 249. ¹H NMR (400 MHz, CD₃OD) δ 9.59 (s, 1H), 8.79 (d, J=2.6 Hz, 1H), 8.51 (d, J=0.7 Hz, 1H), 7.95 (d, J=2.5 Hz, 1H), 7.56-7.44 (m, 2H), 5.15-4.97 (m, 1H), 3.25 (d, J=7.6 Hz, 2H), 3.07 (s, 3H), 1.35 (d, J=6.3 Hz, 6H), 0.95 (d, J=6.7 Hz, 6H), 5.15-4.97 (m, 1H).

### Syntheses of Compound 250

Compounds 250 was synthesized following the urea coupling reaction described above using the appropriate amines.

### Example 72

### Synthesis of N-(4-fluoro-3-(6-(5-fluoro-3-methylpyridin-2-yl)imidazo[1,2-a]pyrimidin-2-yl)phenyl)-2,4-dimethyloxazole-5-carboxamide (251)

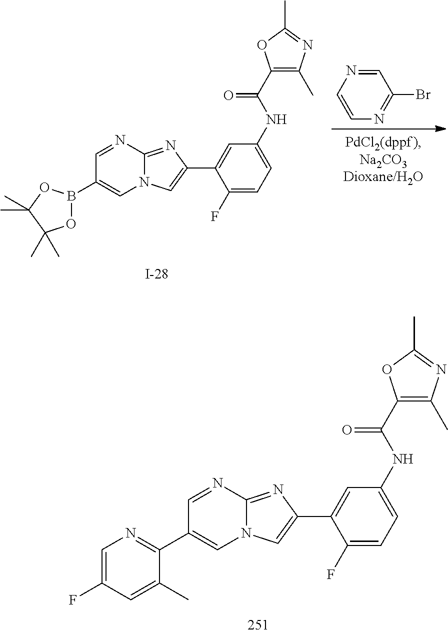

Intermediate I-28 (20 mg, 1 eq.), 2-bromopyrazine (6.6 mg, 1.5 eq.) and 1N Na₂CO₃ (0.2 mL) were taken up in 1,4-dioxane (2 mL), then PdCl₂(dppf) (2 mg, 0.05 eq.) was added under N₂. The solution was sparged with N₂ and stirred overnight at 80° C. HPLC purification yielded compound 251 (2.5 mg). ¹H NMR (400 MHz, CD₃OD) δ 9.79 (d, J=2.4 Hz, 1H), 9.53 (d, J=2.2 Hz, 1H), 9.32 (d, J=1.5 Hz, 1H), 8.84-8.76 (m, 1H), 8.69 (d, J=2.5 Hz, 1H), 8.49 (d, J=3.1 Hz, 1H), 8.40 (dd, J=6.6, 2.6 Hz, 1H), 7.82 (ddd, J=8.8, 4.4, 2.7 Hz, 1H), 7.35 (dd, J=10.8, 9.0 Hz, 1H), 2.57 (s, 3H), 2.48 (s, 3H). MS M/Z=430.4 (M+1).

### Example 73

### Syntheses of Compounds 252 to 286

Compounds 252 to 286 were synthesized following the urea coupling reaction described in Example 71 using the appropriate amines.

### Syntheses of Compounds 287 to 295

Compounds 287 to 295 were synthesized following the urea coupling reaction described above using the intermediate I-10 and the appropriate amines.

### Example 74

### Synthesis of 3,3-difluoro-N-(4-fluoro-3-(6-(pyridin-2-yl)imidazo[1,2-a]pyrimidin-2-yl)phenyl)azetidine-1-carboxamide (296)

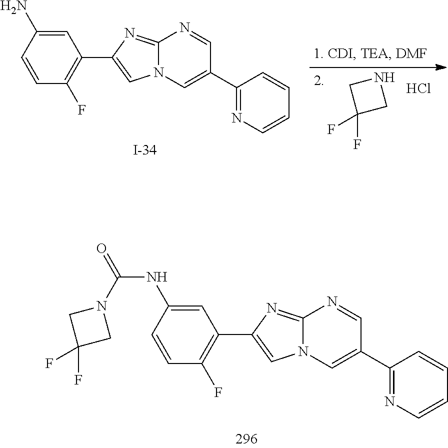

To a solution of CDI (194.5 mg, 1.2 mmol) and TEA (0.8 mL, 5.9 mmol) in DMF (15 mL) was added intermediate I-34 (200 mg, 0.59 mmol) at 0° C. This mixture was stirred at 0° C. for 2 hours, then warmed to room temperature, and stirred for another hour. 3,3-Difluoroazetidine hydrochloride (4 eq, 2.4 mmol) was added to this reaction mixture and stirred overnight. The reaction mixture was diluted with water (50 mL) and extracted with 50% EA/THF (30 mL×2). The combined organic layers were dried with anhydrous sodium sulfate, and concentrated in vacuo. The crude residue was purified by HPLC (CH₃CN/NH₄OH) to give product 296. ¹H NMR (400 MHz, DMSO-d₆) δ 9.71 (s, 1H), 9.28 (s, 1H), 9.11 (s, 1H), 8.73 (d, J=4.27 Hz, 1H), 8.45-8.30 (m, 2H), 8.08 (d, J=7.78 Hz, 1H), 7.98 (td, J=7.72, 1.63 Hz, 1H), 7.72-7.60 (m, 1H), 7.45 (dd, J=7.15, 5.14 Hz, 1H), 7.27 (dd, J=11.04, 9.03 Hz, 1H), 4.41 (t, J=12.80 Hz, 4H). MS m/z=425.0 (M+1).

### Syntheses of Compound 297 to 300

Compounds 297 to 300 were synthesized following the urea coupling reaction described above using intermediate I-36 and the appropriate amines.

### Example 75

### Synthesis of isopropyl (4-fluoro-3-(6-(trifluoromethyl)imidazo[1,2-a]pyrimidin-2-yl)phenyl)carbamate (301)

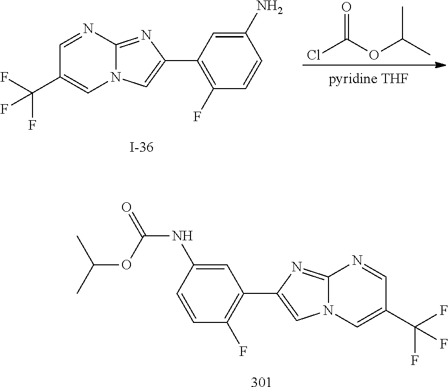

To a solution of compound I-36 (60 mg, 0.203 mmol) in THF (5 mL) was added pyridine (0.065 mL, 0.810 mmol), then the mixture was added isopropyl chloroformate (49.65 mg, 0.405 mmol) at 0° C. and stirred at room temperature for 20 minutes. The mixture was diluted with water (10 mL) and extracted with EA:THF=1:1 (10 mL×2). The combined organic layers were washed with brine (10 mL), dried over sodium sulfate and concentrated. The residue was purified by. HPLC to afford 301 as a yellow solid. (30 mg, yield: 39%).

¹H NMR (DMSO-d₆) δ 9.75 (s, 1H), 9.64 (s, 1H), 8.90 (d, J=4.0 Hz, 1H), 8.45 (d, J=4.0 Hz, 1H), 8.34 (t, J=8.0 Hz, 1H), 7.49 (t, J=8.0 Hz, 1H), 7.29 (t, J=8.0 Hz, 1H), 4.88-4.96 (m, 1H), 1.26 (s, 6H). MS m/z=382.9 (M+1).

### Syntheses of Compound 302 and 303

Compounds 302 and 303 were synthesized following the carbamate coupling reaction described above using the intermediate I-10 and the appropriate chloroformates.

### Syntheses of Compound 304 to 312 and 519

Compounds 304 to 311 and 519 were synthesized following the carbamate coupling reaction described above using the intermediate I-31 and the appropriate chloroformates. Compound 312 was prepared using 1-methylcyclopropyl(4-nitrophenyl) carbonate.

### Synthesis of Compound 313

Compound 313 was synthesized following the carbamate coupling reaction described above using the intermediate I-33 and 2-methoxyethyl chloroformate.

### Synthesis of Compound 314

Compound 314 was synthesized following the carbamate coupling reaction described above using the intermediate I-34 and isopropyl chloroformate.

### Example 76

### Synthesis of N-(4-fluoro-3-(6-(1-methylethylsulfonamido)imidazo[1,2-a]pyrimidin-2-yl)phenyl)-2,4-dimethyloxazole-5-carboxamide (315)

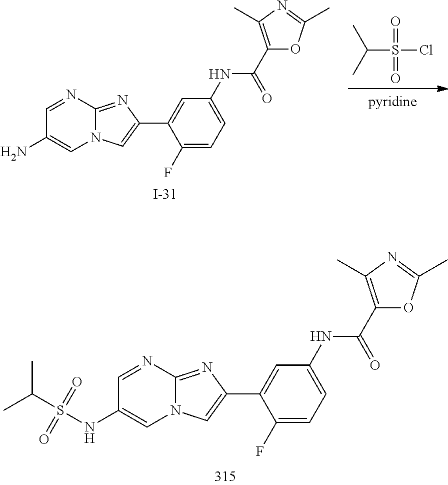

To a solution of 1-31 in pyridine (0.1 M) was added isopropyl sulfonyl chloride (1.2 eq.) and the reaction was allowed to stir overnight at room temperature. The product was concentrated in vacuo and purified by HPLC to give 315. ¹H NMR (400 MHz, DMSO-d₆) δ 10.34 (s, 1H), 10.06 (s, 1H), 8.98 (d, J=2.7 Hz, 1H), 8.70 (dd, J=2.7, 6.9 Hz, 1H), 8.49 (d, J=2.7 Hz, 1H), 8.39 (d, J=4.2 Hz, 1H), 7.86-7.71 (m, 1H), 7.33 (dd, J=9.0, 10.9 Hz, 1H), 2.50 (s, 3H), 2.40 (s, 3H), 1.30 (d, J=6.8 Hz, 6H).

### Example 77

### Synthesis of N-(3-(6-bromoimidazo[1,2-a]pyrimidin-2-yl)-4-Fluorophenyl)methanesulfonamide (316)

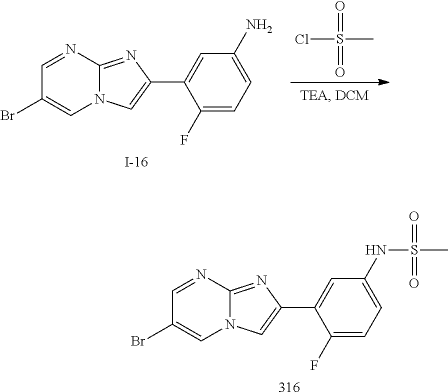

In a 25 mL round bottom flask containing 3-(6-bromoimidazo[1,2-a]pyrimidin-2-yl)-4-fluoroaniline I-16 (0.195 mmol, 60 mg) and triethylamine (0.313 mmol, 44 μl) in DCM (4 mL), was added mesyl chloride (0.215 mmol, 17 μl). The reaction mixture was stirred at room temperature overnight. Water and DCM were added to the reaction mixture and the biphasic solution was transferred into a separating funnel. The organic layer was recovered and after drying over Na₂SO₄ was concentrated in vacuo. The residue obtained was purified by preparative HPLC to yield product 316 as a white powder (15 mg, 20%). ¹H NMR (400 MHz, DMSO-d₆) δ 9.86 (brs, 1H), 9.35 (d, J=2.57 Hz, 1H), 8.65 (d, J=2.57 Hz, 1H), 8.25 (d, J=4.16 Hz, 1H), 8.15 (dd, J=6.66, 2.75 Hz, 1H), 7.31-7.38 (m, 1H), 7.25 (ddd, J=6.63, 4.43, 2.14 Hz, 1H), 2.99 (s, 3H). MS m/z=386.8 (M+1).

### Example 78

### Synthesis of 3-fluoro-N-(4-fluoro-3-(6-phenylimidazo[1,2-a]pyrimidin-2-yl)phenyl)azetidine-1-carboxamide (317)

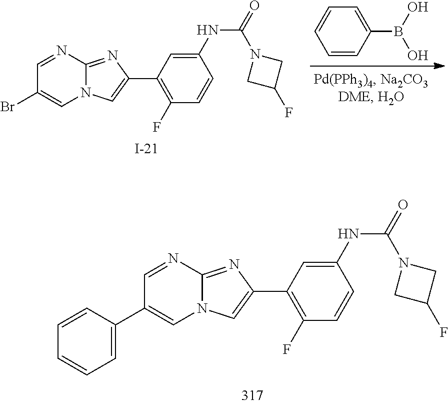

To a mixture of compound I-21 (0.12 mmol), phenyl boronic acid (0.24 mmol, 2 eq.), and sodium carbonate (0.24 mmol, 2 eq.) in DME (2 mL) and H₂O (0.2 mL) was added tetrakis (triphenylphosphine) palladium (13 mg, 12 μmol) under N₂ atmosphere. This mixture was stirred at 90° C. for 5 hours, diluted with 50% EA/THF (50 mL), and washed with brine twice. The organic layer was dried over anhydrous sodium sulfate and concentrated in vacuo. The residue was purified by HPLC to give product 317. ¹H NMR (400 MHz, DMSO-d₆) δ 9.34 (brs, 1H), 8.95 (brs, 1H), 8.83 (s, 1H), 8.39 (d, J=4.27 Hz, 1H), 8.27 (d, J=3.51 Hz, 1H), 7.78 (d, J=7.28 Hz, 2H), 7.72-7.62 (m, 1H), 7.55 (t, J=7.40 Hz, 2H), 7.47 (d, J=7.03 Hz, 1H), 7.24 (t, J=9.91 Hz, 1H), 5.56-5.27 (m, 1H), 4.42-4.22 (m, 2H), 4.12-3.91 (m, 2H). MS m/z=406.0 (M+1).

### Synthesis of Compound 318

Compound 318 was synthesized according to the protocol described above using (3,6-dihydro-2H-pyran-4-yl)boronic acid.

### Syntheses of Compounds 319 and 320

Compounds 319 and 320 were synthesized according to the protocol described above using intermediate I-22 and phenyl boronic acid and (3,6-dihydro-2H-pyran-4-yl)boronic respectively.

### Syntheses of Compounds 321 and 322

Compounds 321 and 322 were synthesized according to the protocol described above using intermediate I-23 and phenyl boronic acid and (3,6-dihydro-2H-pyran-4-yl)boronic respectively.

### Syntheses of Compounds 323 and 324

Compounds 323 and 324 were synthesized according to the protocol described above using intermediate I-24 and phenyl boronic acid and (3,6-dihydro-2H-pyran-4-yl)boronic respectively.

### Syntheses of Compounds 325 and 326

Compounds 325 and 326 were synthesized according to the protocol described above using intermediate I-26 and phenyl boronic acid and (3,6-dihydro-2H-pyran-4-yl)boronic respectively.

### Syntheses of Compounds 327 and 328

Compounds 327 and 328 were synthesized according to the protocol described above using intermediate I-37 and phenyl boronic acid and (3,6-dihydro-2H-pyran-4-yl)boronic respectively.

### Syntheses of Compounds 329 and 330

Compounds 329 and 330 were synthesized according to the protocol described above using intermediate I-18 and phenyl boronic acid and (3,6-dihydro-2H-pyran-4-yl)boronic respectively.

### Syntheses of Compounds 331 and 332

Compounds 331 and 332 were synthesized according to the protocol described above using intermediate I-19 and phenyl boronic acid and (3,6-dihydro-2H-pyran-4-yl)boronic respectively.

### Syntheses of Compounds 333 and 334

Compounds 333 and 334 were synthesized according to the protocol described above using intermediate I-17 and phenyl boronic acid and (3,6-dihydro-2H-pyran-4-yl)boronic respectively.

### Syntheses of Compounds 335 and 336

Compounds 335 and 336 were synthesized according to the protocol described above using intermediate I-44 and phenyl boronic acid and (3,6-dihydro-2H-pyran-4-yl)boronic respectively.

### Syntheses of Compounds 337 and 338

Compounds 337 and 338 were synthesized according to the protocol described above using phenyl boronic acid and intermediate I-45 and I-48 respectively.

### Example 79

### Synthesis of N-(3-(6-cyclopropylimidazo[1,2-a]pyrimidin-2-yl)-4-fluorophenyl)-2,4-dimethyloxazole-5-carboxamide (339)

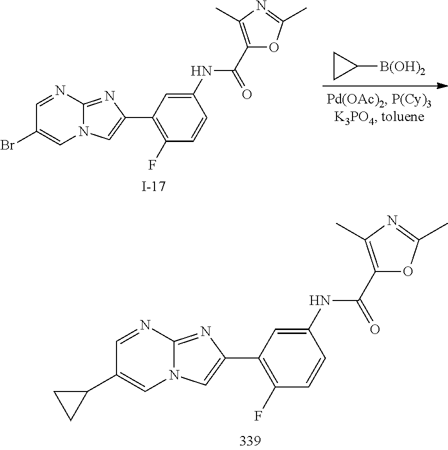

To a solution of intermediate I-17 (300 mg, 0.697 mmol) in toluene (15 mL) and water (1 mL) was added cyclopropyl boronic acid (120 mg, 1.4 mmol), Pd(OAc)₂ (15.6 mg, 0.0698 mmol), P(Cy)₃ (40 mg, 0.14 mmol) and K₃PO₄ (297 mg, 1.4 mmol) at room temperature. The mixture was heated to 120° C., and stirred overnight. The mixture was allowed to cool to room temperature; diluted with water (20 mL), and extracted with 50% EA/THF (50 mL×3). The combined organic layers were washed with brine (50 mL), dried over Na₂SO₄, and concentrated under vacuum. The residue was purified by HPLC to give compound 339 (112 mg, 42%). ¹H NMR (400 MHz, DMSO-d₆) δ 10.39 (s, 1H), 8.97 (s, 1H), 8.72 (s, 1H), 8.63 (d, J=2.8 Hz, 1H), 8.60 (m, 1H), 8.33 (s, 1H), 7.80-7.77 (m, 1H), 7.39-7.44 (m, 1H), 2.10-2.14 (m, 1H), 2.50 (s, 3H), 2.41 (s, 3H), 1.10-1.06 (m, 2H), 0.86-0.85 (m, 2H).

### Example 80

### Syntheses of Compounds 340, 341 and 342

Compounds 340 to 342 were synthesized according to the protocol described in Example 61 using the intermediate I-20 and the appropriate boronic acid.

### Example 81

### Synthesis of tert-butyl 4-(2-(2-chloro-5-(pyrrolidine-1-carboxamido)phenyl)imidazo[1,2-a]pyrimidin-6-yl)-5,6-dihydropyridine-1(2H)-carboxylate (343)

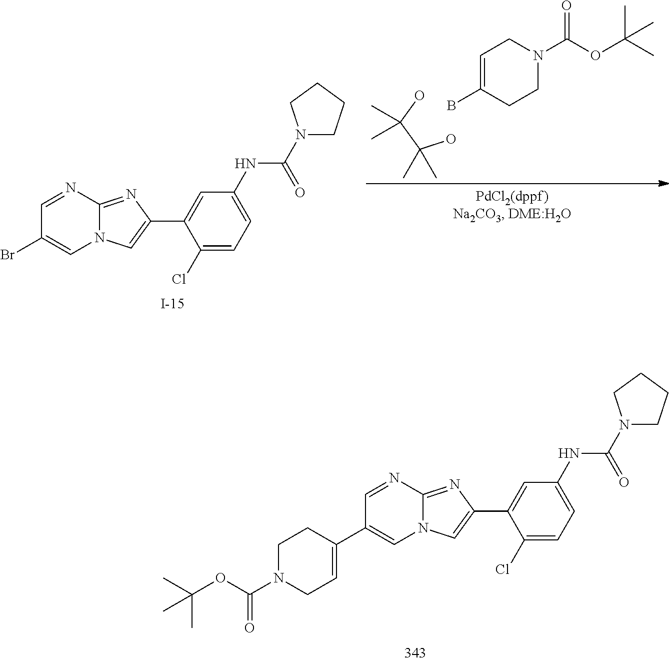

In a vial, compound I-15 (50.4 mg, 0.120 mmol), tert-butyl 4-(4,4,5,5-tetramethyl-1,3,2-dioxaborolan-2-yl)-5,6-dihydropyridine-1(2H)-carboxylate (40.2 mg, 0.132 mmol), sodium carbonate (50.8 mg, 0.479 mmol), and PdCl₂ (dppf) (9.78 mg, 0.012 mmol) were taken up in DME (3 mL) and water (1 mL). The resulting solution was sparged with Ar, and heated to 75° C. overnight. The reaction was quenched with water and extracted with Ethyl Acetate (×3). The organic phases were washed with brine, dried over MgSO₄, and concentrated in vacuo. The crude material was purified by flash column chromatography (0-100% EtOAc/Hexanes), to give the product 343 (17.5 mg, 0.033 mmol, 27.4% yield) as a white solid. ¹H NMR (400 MHz, CD₃OD) δ 8.82-8.69 (m, 2H), 8.29 (s, 1H), 7.96 (d, J=2.6 Hz, 1H), 7.57 (dd, J=8.7, 2.7 Hz, 1H), 7.37 (d, J=8.7 Hz, 1H), 6.33 (s, 1H), 4.19-4.00 (m, 2H), 3.67 (t, J=5.6 Hz, 2H), 3.56-3.41 (m, 4H), 2.53 (tq, J=5.6, 1.9 Hz, 2H), 2.05-1.91 (m, 4H), 1.51 (s, 9H).

### Syntheses of Compounds 344 and 345

Compounds 344 and 345 were synthesized according to the protocol described above using the appropriate boronic acid or ester.

### Syntheses of Compounds 346 to 350 and 359 to 397

Compounds 346 to 350, and 359 to 397 were synthesized according to the protocol described above using the intermediate I-17 and the appropriate boronic acid or ester. Compounds 359 to 397 were prepared using 1,4-dioxane instead of DME as solvent.

### Syntheses of Compounds 351 to 358

Compounds 351 to 358 were synthesized according to the protocol described above using the intermediate I-20 and the appropriate boronic acid or ester.

### Syntheses of Compounds 398 to 410

Compounds 398 to 410 were synthesized according to the protocol described above using 1,4-dioxane as solvent instead of DME, the intermediate I-18 (compound 398), I-19 (compound 399), I-21 (compounds 400 and 401), I-22 (compounds 402 and 403), I-23 (compounds 404 and 405), I-24 (compound 406), I-25 (compounds 406 to 410) and the appropriate boronic acid or ester.

### Example 82

### Synthesis of N-(4-fluoro-3-(6-isopropylimidazo[1,2-a]pyrimidin-2-yl)phenyl)azetidine-1-carboxamide (411)

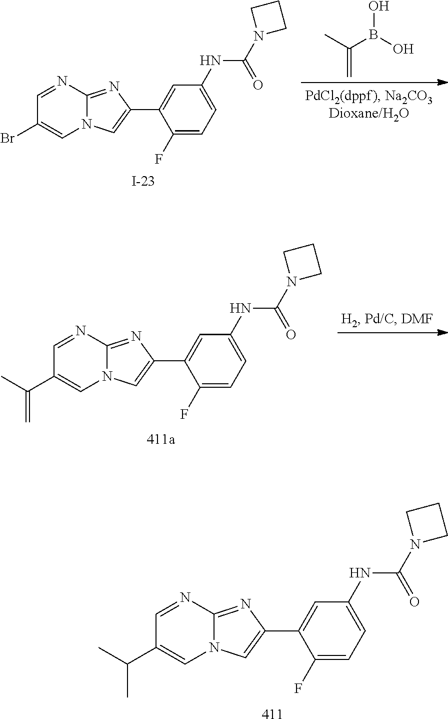

In a vial, compound I-23 (100 mg, 0.256 mmol), isopropenylboronic acid pinacol ester (47.4 mg, 0.282 mmol), sodium carbonate (81 mg, 0.769 mmol), and PdCl₂(dppf) (10.46 mg, 0.013 mmol) were taken up in 1,4-dioxane (3 mL), and the resulting suspension was sparged with Ar, and subsequently heated to 45° C. Additional isopropenylboronic acid pinacol ester (47.4 mg, 0.282 mmol) was added along with PdCl₂(dppf) (10.46 mg, 0.013 mmol) and the temperature was raised to 100° C. overnight. The crude material was evaporated on silica gel and purified by flash column chromatography (0-8% MeOH/DCM, dry load) to give N-(4-fluoro-3-(6-(prop-1-en-2-yl)imidazo[1,2-a]pyrimidin-2-yl)phenyl)azetidine-1-carboxamide 411a (81.3 mg, 0.231 mmol, 90% yield) as a light yellow solid.

In a vial, compound 411a (81.3 mg, 0.231 mmol) was taken up in DMF (4 mL) and the resulting suspension was sparged with argon. To the suspension was added Pd—C (12.31 mg, 0.012 mmol) and the resulting suspension was sparged with hydrogen. After 1 hour, the reaction was filtered and concentrated. The crude material was purified by flash column chromatography (0-7% MeOH/DCM) to give the product 411 (35.8 mg, 0.101 mmol, 43.8% yield) as a white solid.

### Syntheses of Compounds 412 to 418

Compounds 412 to 418 were synthesized according to the protocol described above using the intermediates I-17 (compound 412), I-18 (compound 413), I-19 (compound 414), I-21 (compound 415), I-22 (compound 416), I-24 (compound 417), and I-25 (compound 418).

### Synthesis of Compound 419

Compound 419 was synthesized according to the protocol described above using the intermediates I-20 and (3,6-dihydro-2H-pyran-4-yl)boronic acid.

### Syntheses of Compounds 420 to 423

Compounds 420 to 423 were synthesized according to the protocol described above using the intermediates I-17 and 1-phenylvinylboronic acid (compound 420), (3,6-dihydro-2H-pyran-4-yl)boronic acid (compound 421), 1-cyclopentenylboronic acid pinacol ester (compound 422), or 2-vinylboronic acid pinacol ester (compound 423).

### Synthesis of Compound 424

Compound 424 was synthesized by hydrogenation of compound 330 following the protocol used to convert 411a to 411.

### Example 83

### Synthesis of Compound 425

Compound 425 was synthesized according to the protocol described in Example 81 using the intermediate I-17 and the appropriate boronic acid or ester.

### Example 84

### Synthesis of N-(4-fluoro-3-(6-(1-methyl-1H-imidazol-4-yl)imidazo[1,2-a]pyrimidin-2-yl)phenyl)-2,4-dimethyloxazole-5-carboxamide (426)

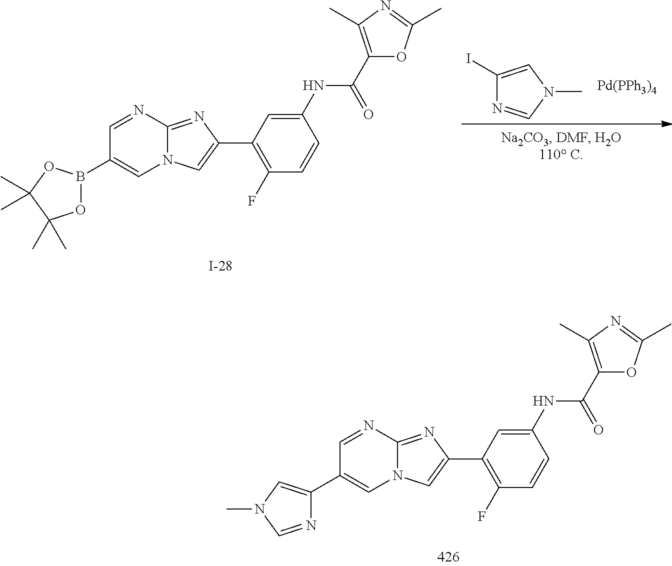

To a solution of compound I-28 (200 mg, 0.42 mmol) in DMF (4 mL) and H₂O (0.4 mL) was added 4-iodo-1-methyl-1H-imidazole (151 mg, 0.63 mmol), Pd(PPh₃)₄ (49 mg, 0.04 mmol) and Na₂CO₃ (89 mg, 0.84 mmol) under N₂ atmosphere. This mixture was heated to 100° C. for 8 hours, diluted with water (20 mL) and extracted with EA/THF (2×40 mL/20 mL), the organic layer was concentrated and purified by HPLC to give compound 426 (7.8 mg, yield, 4%). ¹H NMR (400 MHz, DMSO-d₆) δ 10.36 (brs, 1H), 9.52 (brs, 1H), 9.14 (brs, 1H), 9.02 (brs, 1H), 8.74 (brs, 1H), 8.41 (brs, 1H), 8.19 (brs, 1H), 7.80 (brs, 1H), 7.37 (t, J=9.54 Hz, 1H), 3.93 (br. s., 3H), 2.40 (m, 3H), 2.32 (s, 3H). MS m/z=432.0 (M+1).

### Syntheses of Compound 427 and 428

Compounds 427 and 428 were synthesized according to the protocol described above using 5-iodo-1-methyl-1H-imidazole and I-54.

### Example 85

### Synthesis of N-(3-(6-(1H-imidazol-4-yl)imidazo[1,2-a]pyrimidin-2-yl)-4-fluorophenyl)-2,4-dimethyloxazole-5-carboxamide hydrochloride (429)

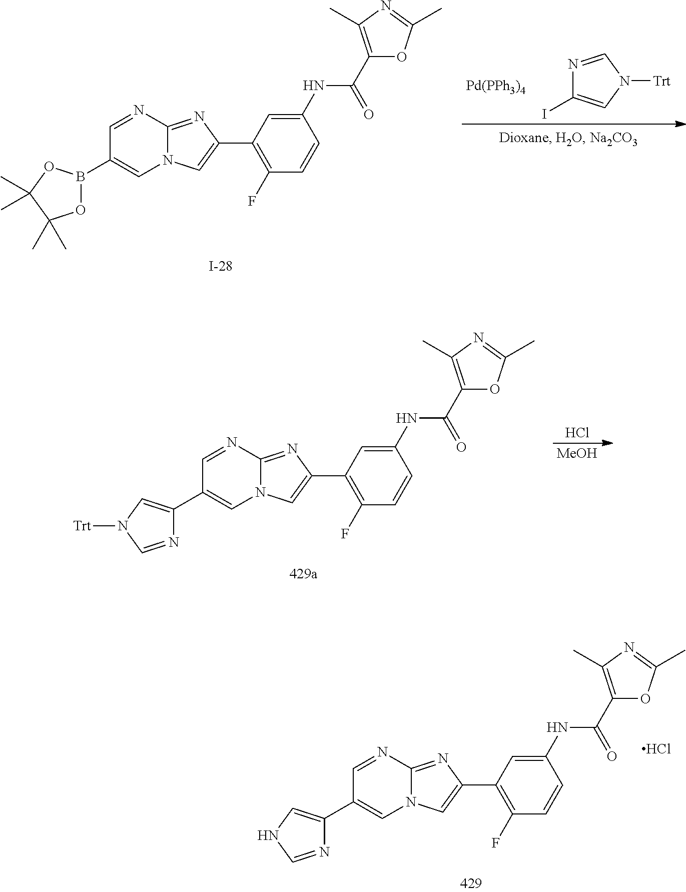

Compound 429a was synthesized according to the protocol described in Example 84 using 4-iodo-1-trityl-1H-imidazole. To a solution of compound 429a (190 mg, 0.29 mmol) in MeOH (10 mL) was then added HCl/MeOH (1.0 mL). This mixture was stirred at room temperature for 4 hours. The solvent was removed and the residue purified by HPLC to give compound 429 (13 mg, 11%). ¹H NMR (400 MHz, DMSO-d₆) δ 12.43 (brs, 1H), 10.33 (s, 1H), 9.30 (d, J=1.76 Hz, 1H), 9.04 (d, J=1.76 Hz, 1H), 8.73 (d, J=4.52 Hz, 1H), 8.35 (d, J=4.02 Hz, 1H), 7.84-7.77 (m, 2H), 7.35-7.30 (m, 1H), 2.49 (s, 3H), 2.41 (s, 3H). MS m/z=418.1 (M+1).

### Example 86

### Synthesis of N-(4-fluoro-3-(6-(5-fluoro-3-methylpyridin-2-yl)imidazo[1,2-a]pyrimidin-2-yl)phenyl)-2,4-dimethyloxazole-5-carboxamide (430)

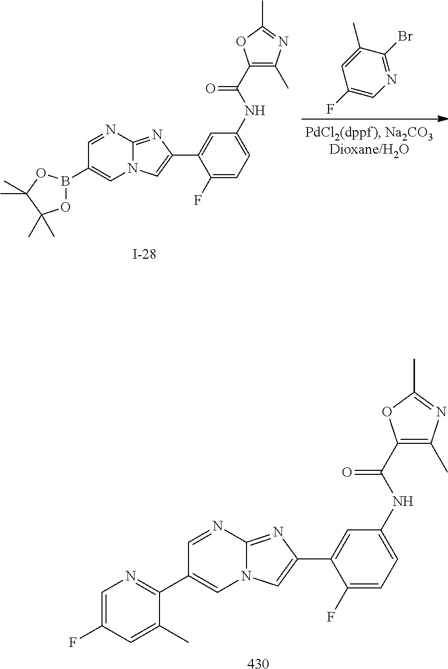

Compound 430 was synthesized according to the protocol described in Example 72 using 2-bromo-5-fluoro-3-methylpyridine. ¹H NMR (400 MHz, CD₃OD) δ 9.31 (d, J=2.3 Hz, 1H), 9.07 (d, J=2.3 Hz, 1H), 8.52 (d, J=2.7 Hz, 1H), 8.47 (d, J=2.9 Hz, 1H), 8.41 (dd, J=6.6, 2.8 Hz, 1H), 7.81 (ddd, J=9.0, 4.4, 2.7 Hz, 1H), 7.73 (dd, J=9.2, 2.7 Hz, 1H), 7.37 (dd, J=10.7, 9.0 Hz, 1H), 2.57 (s, 3H), 2.56 (s, 3H), 2.49 (s, 3H).

### Syntheses of Compounds 431 to 454

Compounds 431 to 454 were synthesized according to the protocol described above and the appropriate aryl bromide.

### Example 87

### Synthesis of N-(4-fluoro-3-(6-(6-methylpyridin-2-yl)imidazo[1,2-a]pyrimidin-2-yl)phenyl)pyrrolidine-1-carboxamide (455)

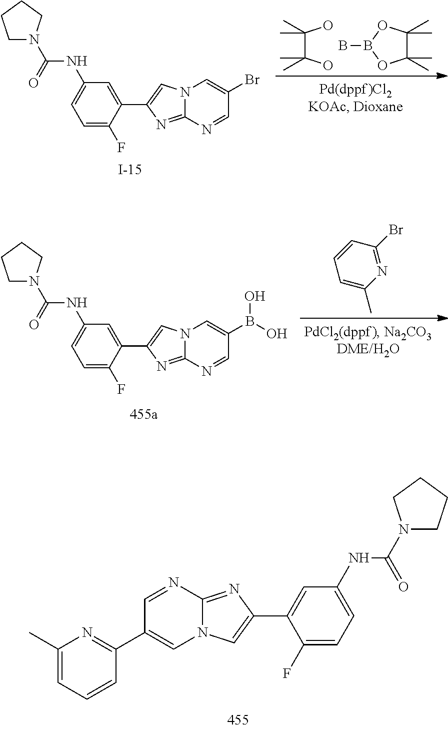

Compound 455a was prepared following the protocol used to synthesize intermediate I-28

Compound 455 was prepared following the protocol used to synthesize compound 343.

### Example 88

### Synthesis of N-(4-chloro-3-(6-(pyridin-2-yl)imidazo[1,2-a]pyrimidin-2-yl)phenyl)pyrrolidine-1-carboxamide (456)

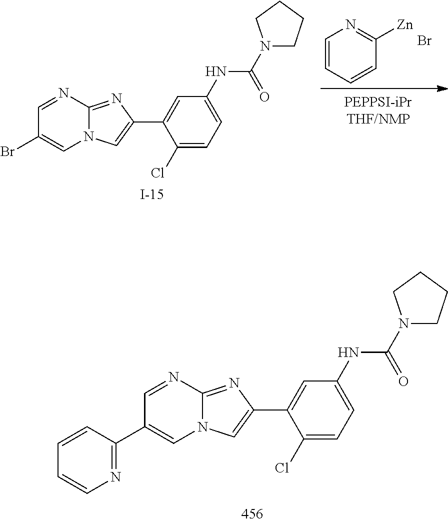

In a vial, intermediate I-15 (20 mg, 0.048 mmol), 2-pyridylzinc bromide (0.114 mL, 0.057 mmol), and [1,3-Bis(2,6-Diisopropylphenyl)imidazol-2-ylidene](3-chloropyridyl) palladium(II) dichloride (PEPPSI-iPr) (1.615 mg, 2.377 μmol) were taken up in THF (1 mL) and NMP (1 mL), and the resulting solution was sparged with Ar and stirred at room temperature overnight. A small amount of anticipated product was observed by LCMS and the reaction was heated to 50° C. for 7 days. The crude material was evaporated on silica gel and purified by flash column chromatography (0-10% MeOH/DCM) to give the product 456 (3.8 mg, 5.44 μmol, 11.45% yield) as a yellow oil.

### Example 89

### Synthesis of N-(3-(6-(4,5-dimethylthiazol-2-yl)imidazo[1,2-a]pyrimidin-2-yl)-4-fluorophenyl)-2,4-dimethyloxazole-5-carboxamide (457)

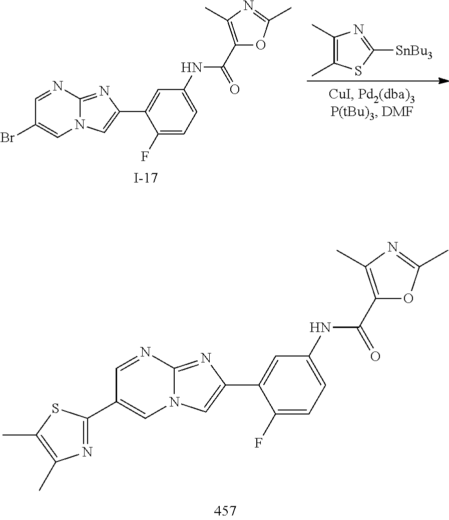

A mixture of 1-17 (1 eq.) and 4,5-dimethyl-2-(tributylstannyl)thiazole was dissolved in DMF (2 mL). P(tBu)₃ (4%) and Cul (4%) were added and the resulting solution was sparged with N₂ for 10 minutes. Pd₂(dba)₃ was added and the mixture was stirred at 80° C. overnight. The reaction mixture was concentrated in vacuo and the residue was purified by HPLC. MS m/z=463.1 (M+1).

### Syntheses of Compounds 458 and 459

Compounds 458 and 459 were synthesized according to the protocol described above using the appropriate tributylstannane.

### Example 90

### Synthesis of 3,3-difluoro-N-(4-fluoro-3-(6-(isopropylamino)imidazo[1,2-a]pyrimidin-2-yl)phenyl)azetidine-1-carboxamide (460)

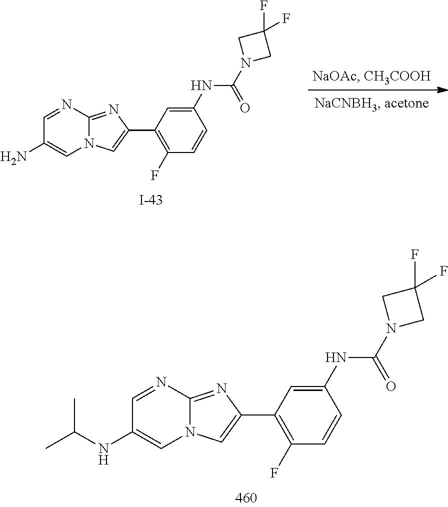

To a solution of compound I-43 (181 mg, 0.5 mmol) in MeOH (20 mL) was added acetone (145 mg, 2.5 mmol), acetic acid (60 mg, 1.0 mmol), and sodium acetate (40.7 mg, 0.5 mmol) at room temperature. After 30 minutes, sodium cyanoborohydride (63 mg, 1.0 mmol) was added, and the reaction mixture was stirred at room temperature for 2 days. The mixture was concentrated and purified by HPLC to give compound 460 (41 mg, yield, 20%). ¹H NMR (400 MHz, DMSO-d₆) δ 9.04 (s, 1H), 8.27-8.24 (m, 2H), 8.06-8.03 (m, 2H), 7.65-7.58 (m, 1H), 7.19 (dd, J=11.04, 9.03 Hz, 1H), 5.68 (d, J=7.53 Hz, 1H), 4.39 (t, J=12.80 Hz, 4H), 3.41-3.38 (m., 1H), 1.19-1.11 (m, 6H). MS m/z=405.1 (M+1).

### Syntheses of Compounds 461, 462

Compounds 461 and 462 were synthesized according to the protocol described above using intermediates I-35 and I-32 respectively.

### Syntheses of Compounds 463 to 465

Compounds 463 to 465 were synthesized according to the protocol described above using intermediate I-31 and the appropriate aldehyde or ketone.

### Example 91

### Synthesis of N-(3-(6-(dimethylamino)imidazo[1,2-a]pyrimidin-2-yl)-4-

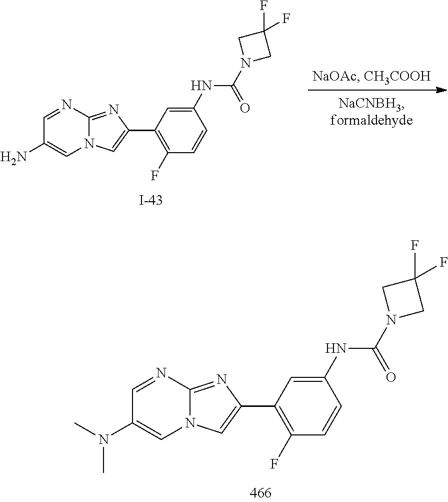

To a solution of compound I-43 (181 mg, 0.5 mmol) in MeOH (20 mL) was added formaldehyde (188 mg, 2.5 mmol), acetic acid (60 mg, 1.0 mmol), and sodium acetate (40.7 mg, 0.5 mmol) at room temperature. After 30 minutes, the sodium cyanoborohydride (63 mg, 1.0 mmol) was added, and this reaction mixture was stirred at room temperature for 2 days. The mixture was concentrated and purified by HPLC to give compound 466 (57 mg, yield, 29%). ¹H NMR (400 MHz, DMSO-d₆) δ 9.06 (brs., 1H), 8.62 (d, J=3.01 Hz, 1H), 8.31-8.33 (m, 2H), 8.11 (d, J=4.52 Hz, 1H), 7.64-7.60 (m, 1H), 7.24-7.19 (m, 1H), 4.40 (t, J=12.80 Hz, 4H), 2.89 (s, 6H). MS m/z=391.1 (M+1).

### Syntheses of Compounds 467, 468, 469, 470

Compounds 467 to 470 were synthesized according to the protocol described above using intermediates I-42, I-32, I-49 and I-31.

### Synthesis of Compound 471

Compound 471 was synthesized according to the protocol described above using intermediate I-31 and acetaldehyde.

### Example 92

### Syntheses of Compounds 472 to 478

Compounds 472 to 478 were synthesized according to the protocol described in Example 90 using intermediate I-33 and the appropriate aldehyde or ketone.

### Syntheses of Compounds 479 to 483

Compounds 479 to 482 were synthesized according to the protocol described above using intermediate I-31 and the appropriate aldehyde or ketone. Compounds 483 was prepared in a similar fashion using compound 481 and formaldehyde.

### Example 93

### Synthesis of isopropyl (2-(2-fluoro-5-(pyridin-2-ylamino)phenyl)imidazo[1,2-a]pyrimidin-6-yl)carbamate (484)

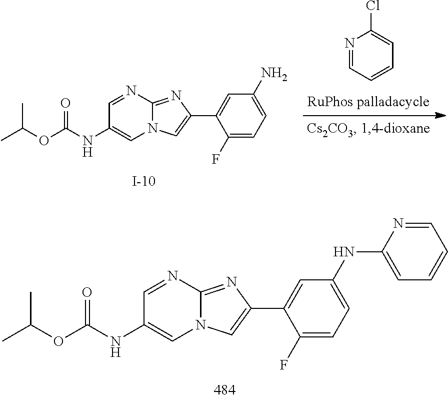

In a vial, compound I-10 (30 mg, 0.09 mmol), 2-chloro pyridine (10.34 mg, 0.09 mmol), RuPhos palladacycle (7.44 mg), and cesium carbonate (60.0 mg, 0.182 mmol) were taken up in 1,4-dioxane (5 mL), and the resulting solution was degassed under vacuum and heated to 120° C. overnight. The reaction mixture was filtered and the residue was purified by reverse phase HPLC. MS m/z=407.1 (M+1).

### Syntheses of Compounds 485 and 486

Compounds 485 and 486 were synthesized according to the protocol described above using 2-chloro-3-(trifluoromethyl)pyridine and 3-bromopyridine respectively.

### Example 94

### Synthesis of isopropyl (2-(2-fluoro-54(3-(trifluoromethoxy)pyridin-2-yl)amino)phenyl)imidazo[1,2-a]pyrimidin-6-yl)carbamate (487)

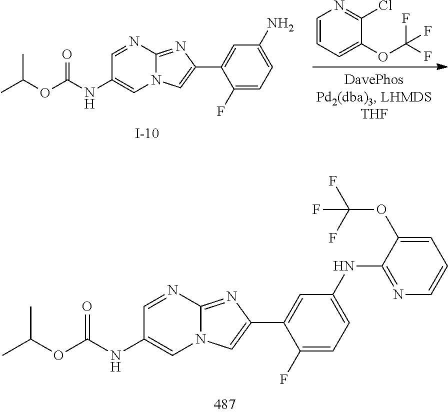

In a vial, intermediate I-10 (40 mg, 0.121 mmol), 2-chloro-3-(trifluoromethoxy)pyridine (28.8 mg, 0.146 mmol), Pd₂(dba)₃ (2.22 mg, 2.429 μmol), and DavePhos (1.14 mg, 2.92 μmol) were taken up in THF (2 mL), and the resulting solution was sparged with argon. To the solution was added LHMDS (0.267 mL, 0.267 mmol), and the reaction was heated to 65° C. overnight. The crude reaction was evaporated on silica gel and purified by flash column chromatography (0-10% MeOH/DCM) The material was further purified by mass-triggered preparatory HPLC to give product 487 (14.1 mg, 0.028 mmol, 23.20% yield) as a white solid. ¹H NMR (400 MHz, CD₃OD) δ 9.27 (s, 1H), 8.46 (d, J=2.7 Hz, 1H), 8.26 (dd, J=6.7, 2.8 Hz, 1H), 8.21 (d, J=3.8 Hz, 1H), 8.06 (dd, J=5.0, 1.5 Hz, 1H), 7.68 [symbol] J=10.9, 8.9 Hz, 1H), 6.83 (dd, J=8.0, 4.9 Hz, 1H), 5.02 (p, J=6.2 Hz, 1H), 1.33 (d, J=6.3 Hz, 6H). MS m/z=491.1 (M+1).

### Syntheses of Compound 497 and 498

Compounds 497 and 498 were synthesized according to the protocol described above using 7-chlorofuro[2,3-c]pyridine and 2-chloro-3-methoxypyridine respectively.

### Syntheses of Compound 488 to 496

Compounds 488 to 494 were synthesized according to the protocol described above using the intermediates I-20 (compounds 488 and 489), I-17 (compounds 490 to 494) and I-15 (compounds 495 and 496) and the corresponding amines.

### Example 95

### Synthesis isopropyl (2-(54(4,5-dihydrooxazol-2-yl)amino)-2-fluorophenyl)imidazo[1,2-a]pyrimidin-6-yl)carbamate (499)

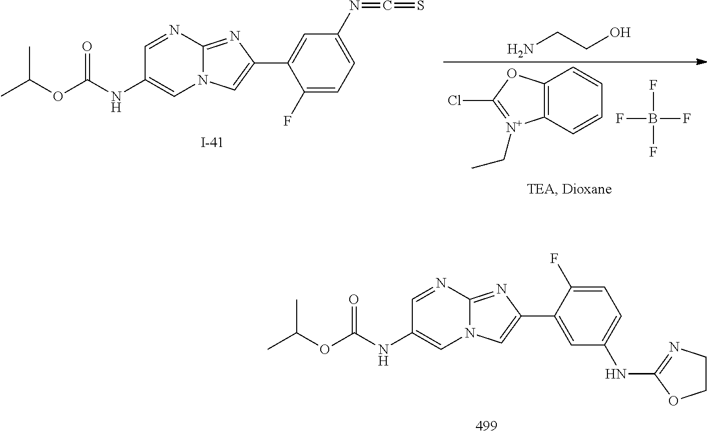

The isothiocyanate I-41 (1 eq.) was placed in a vial with 1,4-dioxane (2 mL). 2-Aminoethanol (1.5 eq.) was added and the mixture was heated at 60° C. for 2 hours. The 1,4-dioxane was evaporated. ACN (1 mL) was added and the mixture was cooled to 0° C. 2-Cl 3-ethyl benzoxazolium tetrafluoroborate (1.5 eq.) was dissolved in ACN (1 mL) and added to the cooled mixture and the resulting mixture was stirred for 1 hour. TEA was added and the mixture stirred at room temperature for 30 minutes. The solvents were evaporated and the residue purified by HPLC to give 499. ¹H NMR (400 MHz, DMSO-d₆) δ 9.92 (s, 1H), 9.21 (s, 1H), 8.41 (d, J=2.7, 1H), 8.26 (d, J=4.2, 1H), 7.75 (s, 1H), 7.27 (t, J=7.3, 1H), 7.21-7.01 (m, 2H), 4.96-4.79 (m, 1H), 4.28 (s, 1H), 4.10 (s, 1H), 3.67 (s, 1H), 1.23 (s, 3H), 1.21 (d, J=2.5, 3H), 1.13 (d, J=6.1, 3H).

### Syntheses of Compounds 500 to 511

Compounds 500 to 511 were synthesized according to the protocol described above using the intermediate I-41 (compound 500 to 503), or 1-40 (compound 504 to 511) and

[symbol]

### Example 96

### Synthesis of benzyl (2-(2-fluoro-5-(pyrrolidine-1-carboxamido)phenyl)imidazo[1,2-a]pyrimidin-6-yl)carbamate (512)

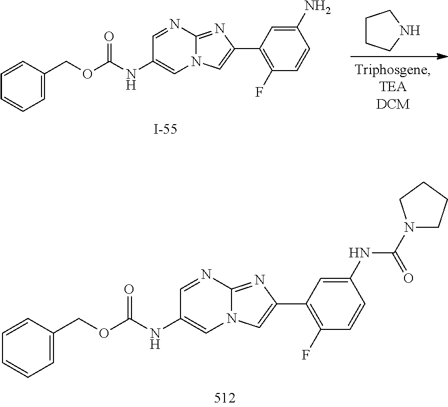

In a 50 mL round bottom flask containing the intermediate I-55 (0.371 mmol, 140 mg) and triphosgene (0.371 mmol, 110 mg) in DCM at 0° C., triethylamine (0.371 mmol, 60 μl) was added. The reaction mixture was stirred at 0° C. for 10 minutes and pyrrolidine (1.113 mmol, 92 μl) was slowly added; fumes were released. The mixture was slowly brought to room temperature and stirred for 1 hour. The reaction mixture was diluted with water and DCM and transferred into a separating funnel. The organic layer was recovered. After drying over Na₂SO₄, the combined organic layers were adsorbed onto silica and purified by flash column chromatography (DCM/MeOH) to afford the product 512 as a pale solid (140 mg, 79%). ¹H NMR (400 MHz, DMSO-d₆) δ 10.18 (brs, 1H), 9.28 (brs, 1H), 8.49 (d, J=2.69 Hz, 1H) 8.30-8.39 (m, 3H), 7.63 (ddd, J=8.93, 4.46, 2.87 Hz, 1H), 7.33-7.49 (m, 5H), 7.18 (dd, J=10.94, 8.99 Hz, 1H), 5.21 (s, 2H), 3.39 (t, J=6.66 Hz, 4H), 1.82-1.90 (m, 4H). MS m/z=476.1 (M+1).

### Syntheses of Compounds 513 and 514

Compounds 513 and 514 were synthesized by following the urea coupling reaction described above using intermediates I-56 and I-35 respectively.

### Syntheses of Compounds 515 to 517

Compounds 515 to 517 were synthesized by following the urea coupling reaction described above using intermediate I-16 and morpholine, N-methyl piperazine and methanol respectively.

### Example 97

### Synthesis of Compound 518

Compound was synthesized following the carbamate coupling reaction described in Example 75 using the intermediate I-55, isopropyl chloroformate, triethylamine as the base and DCM as the solvent.

### Synthesis of Compound 519

Compound 519 was synthesized following the carbamate coupling reaction described above using the intermediate I-31 and the appropriate chloroformates.

### Example 98

### Synthesis N-(4-chloro-3-(6-(2-oxooxazolidin-3-yl)imidazo[1,2-a]pyrimidin-2-yl)phenyl)-2,4-dimethyloxazole-5-carboxamide (520)

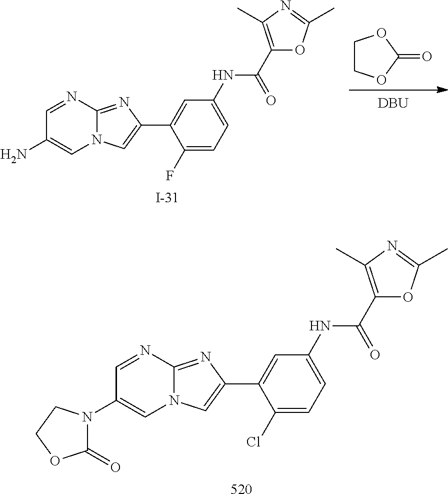

Intermediate I-31 (30 mg, 1 eq.), 1,3-dioxolan-2-one (34.5 mg, 5 eq.) and DBU (0.012 mL, 1 eq.) were added into a vial and sealed. The atmosphere in the vial was evacuated of air and purged with nitrogen. The vial was then heated to 100° C. for 2 hours. The reaction was then quenched with a little bit of water and extracted with DCM thrice. The organic extracts were combined, dried over MgSO₄, filtered and evaporated in vacuo. The residue was purified by preparative reverse phase HPLC to provide compound 520 (5 mg, 14% yield). ¹H NMR (400 MHz, DMSO-d₆) δ 10.42 (s, 1H), 9.28 (d, J=2.8 Hz, 1H), 8.93 (d, J=2.8 Hz, 1H), 8.76 (d, J=2.7 Hz, 1H), 8.68 (s, 1H), 7.84 (dd, J=2.7 Hz, 8.8, 1 H), 7.53 (d, J=8.7 Hz, 1H), 4.54 (dd, J=6.9, 9.0 Hz, 2H), 4.15 (dd, J=7.2, 8.9 Hz, 2H), 2.50 (s, 3H), 2.40 (s, 3H).

### Example 99

### Synthesis of N-(4-fluoro-3-(6-(hydrazinecarboxamido)imidazo[1,2-a]pyrimidin-2-yl)phenyl)pyrrolidine-1-carboxamide (521)

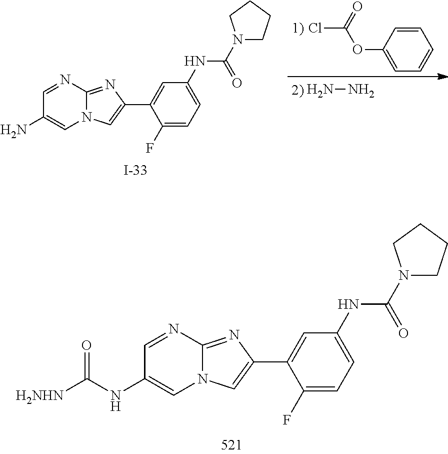

To a stirred solution of intermediate I-33 (100 mg, 1 eq.) in pyridine (2 mL) was added [symbol] stirred overnight at 70° C. HPLC purification led to 60 mg of intermediate phenyl (2-(2-fluoro-5-(pyrrolidine-1-carboxamido)phenyl)imidazo[1,2-a]pyrimidin-6-yl)carbamate. This intermediate was dissolved in EtOH (3 mL), hydrazine (10 uL) was added, and the mixture was heated up to 80° C. overnight. HPLC purification yields 25 mg of compound 521 as a TFA salt. MS m/z=399.0 (M+1).

### Synthesis of Compound 522

Compound 522 was synthesized according to the protocol described above using isopropylhydrazine.

### Example 100

### Synthesis of Compound 523

Compound 523 was synthesized according to a protocol similar to the one described in Example 90 using intermediate I-10 and sodium acetoxyhydride.

### Example 101

### Synthesis of Compound 524

Compound 524 was synthesized according to the protocol described in Example 95 using the intermediate I-40 and the corresponding amino alcohol.

### Example 102

### Synthesis of N-(3-(6-acetamidoimidazo[1,2-a]pyrimidin-2-yl)-4-fluorophenyl)-2,4-dimethyloxazole-5-carboxamide (525)

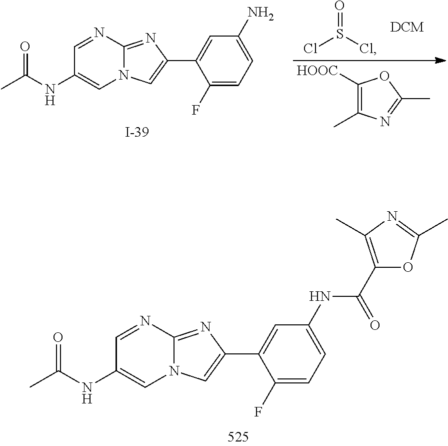

To a solution of 2,4-dimethyloxazole-5-carboxylic acid (9.9 mg, 1 eq.) in DCM (0.5 mL) was added thionyl chloride (12.5 mg, 1.5 eq.) and the mixture was allowed to stir at 50° C. overnight. Thereafter the reaction was dried in vacuo thoroughly and added to it a solution of intermediate I-39 (20 mg, 1 eq.) in pyridine (2 mL). The resultant mixture was allowed to stir at room temperature for 1 hour. The reaction was deemed completed and proceeded to purification via reverse phase prep HPLC to yield compound 525 (3.3 mg, 11.5%). ¹H NMR (400 MHz, DMSO-d₆) δ 10.67 (s, 1H), 10.34 (s, 1H), 9.65 (d, J=2.6 Hz, 1H), 8.78 (d, J=2.6 Hz, 1H), 8.71 (dd, J=2.8, 6.9 Hz, 1H), 8.42 (d, J=4.2 Hz, 1H), 7.79 (ddd, J=2.8, 4.4, 8.8 Hz, 1H), 7.32 (dd, J=9.0, 10.9 Hz, 1H), 2.53 (s, 5H), 2.41 (d, J=7.5 Hz, 6H).

### Example 103

### Synthesis of isopropyl (2-(3-(5-methyl-1,3,4-oxadiazole-2-carboxamido)phenyl)imidazo[1,2-a]pyrimidin-6-yl)carbamate (526)

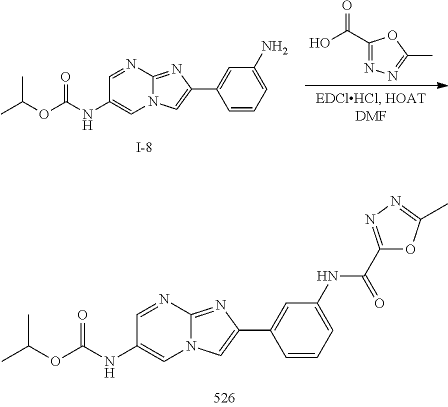

To a solution of 5-methyl-1,3,4-oxadiazole-2-carboxylic acid (12.4 mg, 0.096 mmol) in DMF (2 mL) was added N-(3-Dimethylaminopropyl)-N′-ethylcarbodiimide hydrochloride (18.5 mg, 0.096 mmol) and HOAT (13.1 mg, 0.096 mmol). The reaction was stirred at room temperature for 30 minutes then isopropyl (2-(3-aminophenyl)imidazo[1,2-a]pyrimidin-6-yl)carbamate I-8 (20 mg, 0.064 mmol) was added and stirring continued for 1.5 hours. The reaction mixture was diluted with Ethyl Acetate and washed with sat NaHCO₃ and brine. The organic layer was dried over magnesium sulfate, filtered and reduced to dryness. The crude product was purified by HPLC to afford isopropyl (2-(3-(5-methyl-1,3,4-oxadiazole-2-carboxamido)phenyl)imidazo[1,2-a]pyrimidin-6-yl)carbamate 526 (5 mg) as white solid. MS m/z 422.4 (M+1).

### Syntheses of Compounds 527 and 528

Compound 527 and 528 were synthesized following the amide coupling reaction described above using intermediate I-9 and the appropriate carboxylic acid.

### Example 104

### Synthesis of tert-butyl (2-(5-acetamido-2-fluorophenyl)imidazo[1,2-a]pyrimidin-6-yl)carbamate (529)

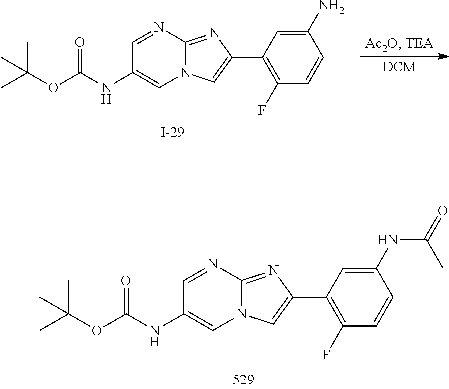

In a 50 mL round bottom flask containing tert-butyl (2-(5-amino-2-fluorophenyl)imidazo[1,2-a]pyrimidin-6-yl)carbamate I-29 (0.233 mmol, 80 mg) dissolved in DCM (5 mL), triethylamine (0.466 mmol, 65 μl) was added followed by acetic anhydride (0.536 mmol, 45 μl). The reaction mixture was stirred at room temperature for 1 hour. Upon completion, the solution obtained was concentrated in vacuo. The residue was triturated in water. Filtration over Buchner gave the entitled product as a pale brown solid (62 mg, 66%). ¹H NMR (400 MHz, DMSO-d₆) δ 10.10 (s, 1H), 9.78 (brs, 1H), 9.30 (brs, 1H), 8.48 (d, J=2.69 Hz, 1H), 8.41 (dd, J=6.85, 2.81 Hz, 1H), 8.33 (d, J=4.16 Hz, 1H), 7.66 (ddd, J=8.83, 4.43, 2.87 Hz, 1H), 7.24 (dd, J=11.00, 9.05 Hz, 1H), 2.06 (s, 3H), 1.51 (s, 9H). MS m/z=386.9 (M+1).

### Example 105

### Synthesis of isopropyl (2-(2-chloro-5-(1,3-dioxoisoindolin-2-yl)phenyl)imidazo[1,2-a]pyrimidin-6-yl)carbamate (530)

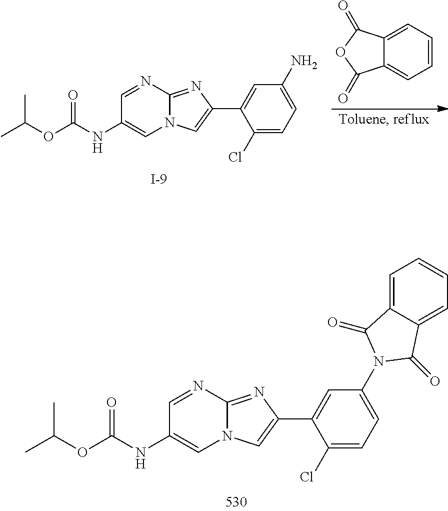

A mixture of intermediate I-9 (100 mg, 1 eq.), isobenzofuran-1,3-dione (86 mg, 2 eq.) and TEA (0.1 mL) in toluene (5 mL) was heated overnight at 80° C. The reaction mixture was concentrated in vacuo and purified by flash chromatography to give compound 530 (50 mg, 36% yield). MS m/z=475.9 (M+1).

### Example 106

### Synthesis of N-(3-(6-bromoimidazo[1,2-a]pyrimidin-2-yl)-4-fluorophenyl)formamide (531)

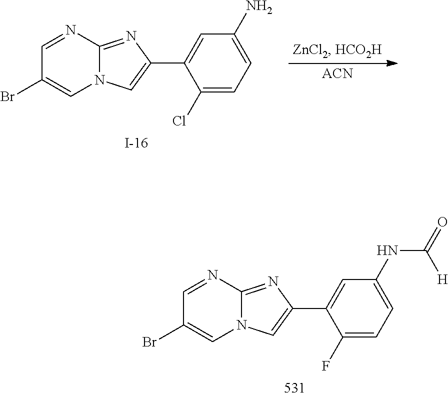

In a 25 mL round bottom flask containing 3-(6-bromoimidazo[1,2-a]pyrimidin-2-yl)-4-fluoroaniline I-16 (0.195 mmol, 60 mg) and zinc chloride (0.037 mmol, 5 mg) in acetonitrile (3 mL), formic acid was added (0.977 mmol, 37 μl). The reaction mixture was stirred at 70° C. for 16 hours. Water and Ethyl Acetate were added to the reaction mixture and the biphasic solution was transferred into a separating funnel. The organic layer was recovered and after drying over Na₂SO₄ was concentrated in vacuo. The residue obtained was purified by preparative HPLC to yield product 531 as a white powder (12 mg, 18%). ¹H NMR (400 MHz, DMSO-d₆) δ 10.37 (s, 1H), 9.35 (d, J=2.57 Hz, 1H), 8.65 (d, J=2.57 Hz, 1H), 8.51 (dd, J=6.79, 2.75 Hz, 1H), 8.31 (d, J=1.83 Hz, 1H), 8.23-8.27 (m, 1H), 7.68 (ddd, J=8.83, 4.49, 2.81 Hz, 1H), 7.32 (dd, J=11.13, 8.93 Hz, 1H). MS m/z=336.1 (M+1).

### Example 107

### Synthesis of tert-butyl (2-(2-fluoro-5-formamidophenyl)imidazo[1,2-a]pyrimidin-6-yl)carbamate (532)

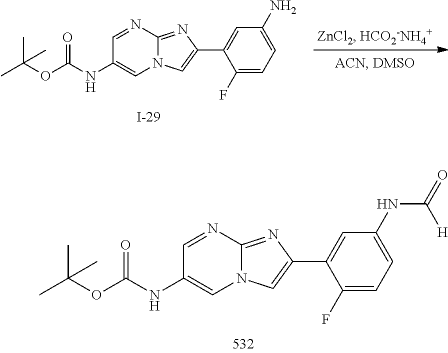

In a 2 mL microwave vial containing tert-butyl (2-(5-amino-2-fluorophenyl)imidazo[1,2-a]pyrimidin-6-yl)carbamate I-29 (0.146 mmol, 50 mg), ammonium formate (0.291 mmol, 18 mg) and zinc chloride (0.073 mmol, 10 mg) were added followed by anhydrous acetonitrile (1 mL) and DMSO (0.1 mL). The reaction mixture was stirred at 130° C. for 2 h 30 under microwave irradiations. Water and Ethyl Acetate were added to the reaction mixture and the biphasic solution was transferred into a separating funnel. The organic layer was recovered and after drying over Na₂SO₄ was concentrated in vacuo. The residue obtained was purified by preparative HPLC to yield product 532 as a white powder (8 mg, 14%). ¹H NMR (400 MHz, DMSO-d₆) δ 10.34 (s, 1H), 9.79 (brs, 1H), 9.30 (brs, 1H), 8.48 (d, J=2.69 Hz, 1H), 8.45 (dd, J=6.72, 2.69 Hz, 1H), 8.34-8.38 (m, 1H), 8.30 (d, J=1.71 Hz, 1H), 7.65 (ddd, J=8.83, 4.37, 2.93 Hz, 1H), 7.28 (dd, J=11.07, 8.86 Hz, 1H), 1.51 (s, 9H). MS m/z=372.9 (M+1).

### Example 108

### Synthesis of N-(4-fluoro-3-(6-formamidoimidazo[1,2-a]pyrimidin-2-yl)phenyl)pyrrolidine-1-carboxamide (533)

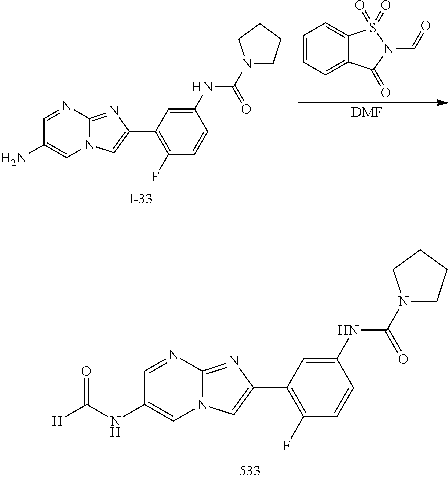

In a 5 mL microwave vial containing N-(3-(6-aminoimidazo[1,2-a]pyrimidin-2-yl)-4-fluorophenyl)pyrrolidine-1-carboxamide I-33 (0.118 mmol, 40 mg) and 3-oxobenzo[d]isothiazole-2(3H)-carbaldehyde 1,1-dioxide (0.235 mmol, 50 mg), anhydrous DMF (2 mL) was added. The reaction mixture was stirred under microwave conditions at 100° C. for 40 minutes. At this stage, the mixture was concentrated in vacuo and purification via preparative HPLC of the residue allowed the isolation of product 533 as a pale white solid (1 mg, 2%). ¹H NMR (400 MHz, CD₃OD) δ 9.57 (d, J=2.57 Hz, 1H), 8.55 (s, 1H), 8.53 (d, J=2.57 Hz, 1H), 8.43 (s, 1H), 8.23 (d, J=3.91 Hz, 1H), 7.99 (dd, J=6.72, 2.69 Hz, 1H), 7.58 (ddd, J=8.93, 4.52, 2.81 Hz, 1H), 7.16 (dd, J=11.00, 8.93 Hz, 1H), 3.47-3.52 (m, 4H), 2.00 (s, 4H). MS m/z=369.0 (M+1).

### Example 109

### Synthesis of N-(4-fluoro-3-(6-(5-oxo-1H-1,2,4-triazol-4(5H)-yl)imidazo[1,2-a]pyrimidin-2-yl)phenyl)pyrrolidine-1-carboxamide (534)

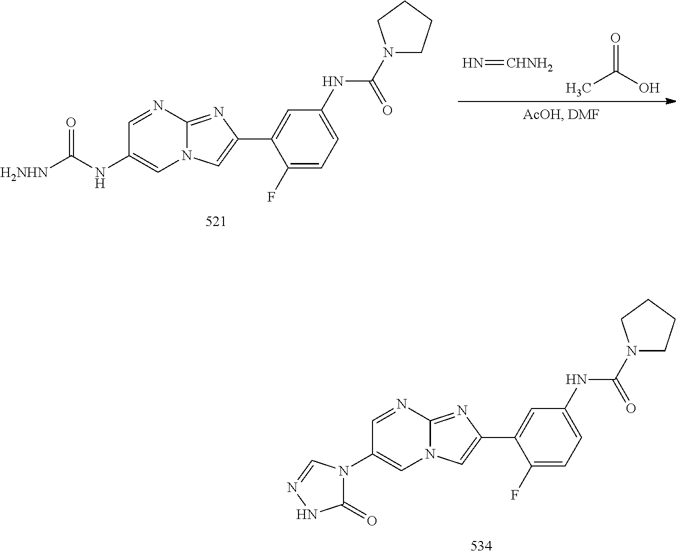

To a mixture of compound 521 (15 mg, 1 eq.) and formamidine acetate (3 eq.) in DMF (3 mL) was added acetic acid (0.2 mL). The mixture was heated at 80° C. overnight, concentrated in vacuo and purified by HPLC to give compound 534 (3 mg, 20% yield). ¹H NMR (400 MHz, CD₃OD) δ 9.38 (d, J=2.6 Hz, 1H), 8.88 (d, J=2.6 Hz, 1H), 8.35 (d, J=3.6 Hz, 1H), 8.25 (s, 1H), 8.06 (dd, J=6.6, 2.8 Hz, 1H), 7.60 (ddd, J=8.9, 4.4, 2.8 Hz, 1H), 7.20 (dd, J=10.9, 9.0 Hz, 1H), 3.56-3.43 (m, 4H), 2.05-1.94 (m, 4H).

### Synthesis of Compound 535

Compound 535 was synthesized in a same fashion as compound 532 using intermediate I-8 instead of I-33.

### Example 110

### Synthesis of N-(4-fluoro-3-(6-methoxyimidazo[1,2-a]pyrimidin-2-yl)phenyl)pyrrolidine-1-carboxamide (536)

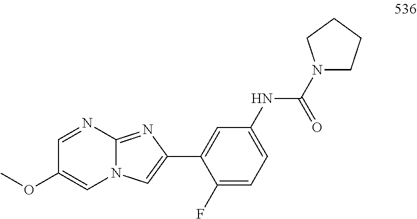 [SMILES: COc1cnc2nc(-c3cc(NC(=O)N4CCCC4)ccc3F)cn2c1]

Compound 536 was synthesized in a similar fashion as intermediate I-20 using 2-amino-5-methoxypyrimidine instead of 2-amino-5-bromopyridine as starting material. ¹H NMR (400 MHz, CD₃OD) δ 8.55 (d, J=3.1 Hz, 1H), 8.42 (d, J=2.9 Hz, 1H), 8.13 (d, J=3.8 Hz, 1H), 7.93 (dd, J=6.7, 2.7 Hz, 1H), 7.56 (ddd, J=8.9, 4.4, 2.8 Hz, 1H), 7.14 (dd, J=11.0, 8.9 Hz, 1H), 3.91 (s, 3H), 3.55-3.43 (m, 4H), 2.04-1.93 (m, 4H). MS m/z=356.1 (M+1).

### Example 111

### Synthesis of N-(4-fluoro-3-(6-(prop-1-en-2-yl)imidazo[1,2-a]pyrimidin-2-yl)phenyl)-2,4-dimethyloxazole-5-carboxamide (537)

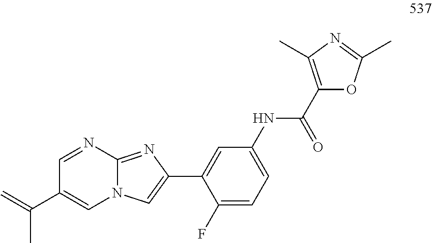 [SMILES: C=C(C)c1cnc2nc(-c3cc(NC(=O)c4oc(C)nc4C)ccc3F)cn2c1]

Compound 537 was synthesized in a similar fashion as intermediate I-17 in Example 11 using 5-(prop-1-en-2-yl)pyrimidin-2-amine I-52 as starting material. ¹H NMR (400 MHz, CD₃OD) δ 8.94 (d, J=2.5 Hz, 1H), 8.88 (d, J=2.5 Hz, 1H), 8.30-8.19 (m, 2H), 7.84 (ddd, J=8.9, 4.4, 2.8 Hz, 1H), 7.24 (dd, J=10.9, 8.9 Hz, 1H), 5.67 (s, 1H), 5.32 (d, J=1.8 Hz, 1H), 2.56 (s, 3H), 2.47 (s, 3H), 2.23 (s, 3H). MS m/z=392.1 (M+1).

### Example 112

### Synthesis of N-(4-fluoro-3-(6-(piperidin-4-yl)imidazo[1,2-a]pyrimidin-2-yl)phenyl)-2,4-dimethyloxazole-5-carboxamide (538)

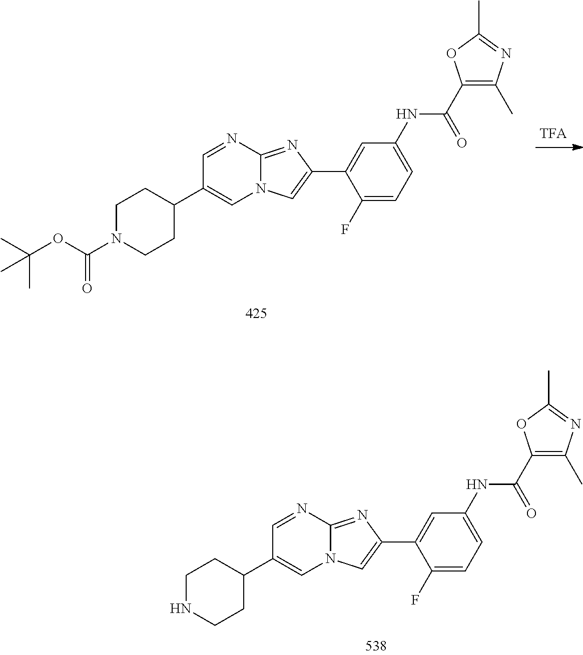 [SMILES: Cc1nc(C)c(C(=O)Nc2ccc(F)c(-c3cn4cc(C5CCN(C(=O)OC(C)(C)C)CC5)cnc4n3)c2)o1 ; Cc1nc(C)c(C(=O)Nc2ccc(F)c(-c3cn4cc(C5CCNCC5)cnc4n3)c2)o1]

TFA (5 mL) was added to tert-butyl 4-(2-(5-(2,4-dimethyloxazole-5-carboxamido)-2-fluorophenyl)imidazo[1,2-a]pyrimidin-6-yl)piperidine-1-carboxylate 425 (0.2 g) and the resulting mixture was stirred for 2 hours. After the evaporation of the solvent, the crude product was dissolved in CH₂Cl₂ and saturated NaHCO₃. The organic phase was isolated and evaporated to give N-(4-fluoro-3-(6-(piperidin-4-yl)imidazo[1,2-a]pyrimidin-2-yl)phenyl)-2,4-dimethyloxazole-5-carboxamide 538 as an oil and was used in the following step without further purification. MS m/z 345.2 (M+1).

### Example 113

### Synthesis of methyl 2-(4-(2-(5-(2,4-di methyloxazole-5-carboxamido)-2-fluorophenyl)imidazo[1,2-a]pyrimidin-6-yl)piperidin-1-yl)acetate (539)

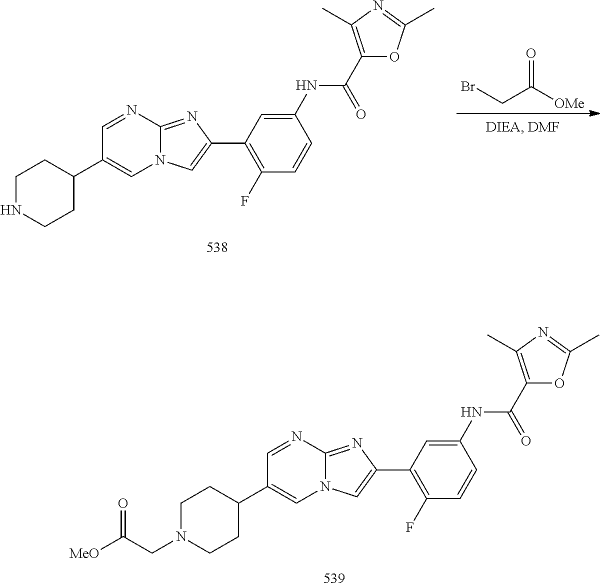 [SMILES: Cc1nc(C)c(C(=O)Nc2ccc(F)c(-c3cn4cc(C5CCNCC5)cnc4n3)c2)o1 ; COC(=O)CBr ; COC(=O)CN1CCC(c2cnc3nc(-c4cc(NC(=O)c5oc(C)nc5C)ccc4F)cn3c2)CC1]

Methyl 2-bromoacetate (16 mg) was added to a stirred mixture of N-(4-fluoro-3-(6-(piperidin-4-yl)imidazo[1,2-a]pyrimidin-2-yl)phenyl)-2,4-dimethyloxazole-5-carboxamide 538 (40 mg) and iPr₂NEt (35.7 mg). The resulting mixture was heated for 2 hours at 50° C. before it was purified directly by HPLC to give methyl 2-(4-(2-(5-(2,4-dimethyloxazole-5-carboxamido)-2-fluorophenyl)imidazo[1,2-a]pyrimidin-6-yl)piperidin-1-yl)acetate 539. ¹H NMR (400 MHz, DMSO-d₆) δ 10.36 (s, 1H), 8.93 (s, 1H), 8.72 (dd, J=7.0, 2.6 Hz, 1H), 8.32 (d, J=3.9 Hz, 1H), 7.78 (ddd, J=9.0, 4.5, 2.8 Hz, 1H), 7.35 (dd, J=10.9, 8.9 Hz, 1H), 4.31 (s, 2H), 3.80 (s, 3H), 3.74-3.58 (m, 2H), 3.34-3.17 (m, 2H), 2.99 (d, J=12.9 Hz, 1H), 2.50 (s, 3H), 2.40 (s, 3H), 2.27-2.12 (m, 2H), 2.10-1.96 (m, 2H).

### Syntheses of Compound 540 and 541

Compounds 540 and 541 were synthesized following the protocol described above using 1-bromo-2-methoxyethanol and 2-bromoethanol as alkylating agents respectively.

### Example 114

### Synthesis of N-(4-fluoro-3-(6-(1-(2-hydroxyacetyl)piperidin-4-yl)imidazo[1,2-a]pyrimidin-2-yl)phenyl)-2,4-dimethyloxazole-5-carboxamide (542)

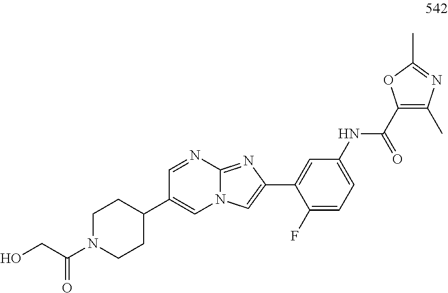 [SMILES: Cc1nc(C)c(C(=O)Nc2ccc(F)c(-c3cn4cc(C5CCN(C(=O)CO)CC5)cnc4n3)c2)o1]

Compound 542 was synthesized following the amide coupling protocol described in Example 51 using compound 538 and 2-hydroxyacetic acid.

### Example 115

### Synthesis of N-(4-fluoro-3-(6-(4-hydroxybut-1-yn-1-yl)imidazo[1,2-a]pyrimidin-2-yl)phenyl)-2,4-dimethyloxazole-5-carboxamide (543)

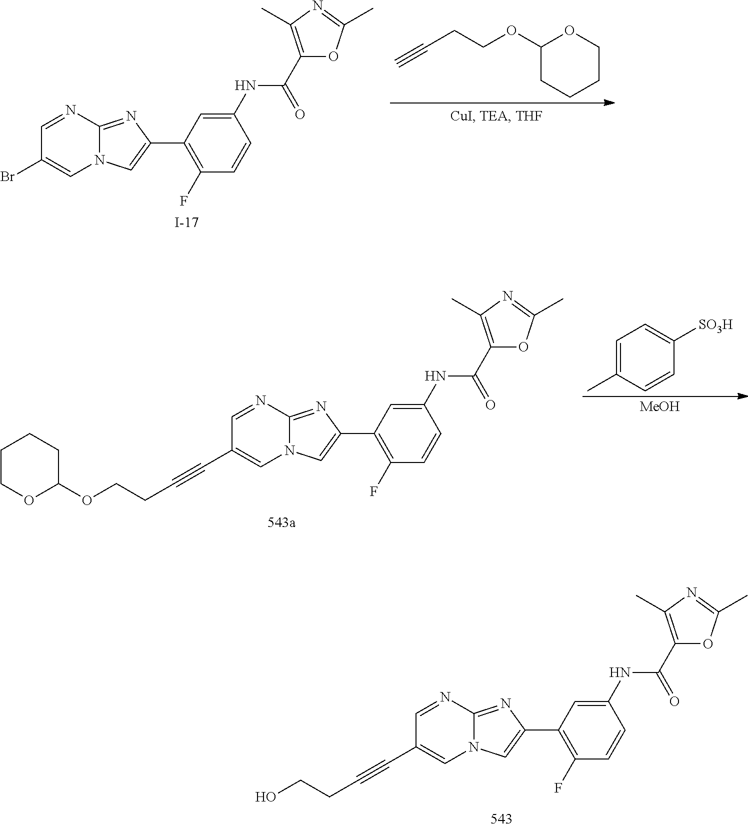

### Synthesis of Compound 543a

[1,1-Bis(diphenylphosphino)ferrocene]dichloropalladium(II), complex with dichloromethane (19 mg) was added to a degassed mixture of Cul (9 mg), N-(3-(6-bromoimidazo[1,2-a]pyrimidin-2-yl)-4-fluorophenyl)-2,4-dimethyloxazole-5-carboxamide I-17 (0.1 g), 2-(but-3-yn-1-yloxy)tetrahydro-2H-pyran (0.11 g) and triethylamine (0.12 g) in THF. The resulting mixture was stirred at 80° C. for 5 hours. The mixture was poured into EtOAc and saturated NH₄Cl. The organic phase was isolated and residue was purified over silica using CH₂Cl₂ and MeOH to give N-(4-fluoro-3-(6-(4-((tetrahydro-2H-pyran-2-yl)oxy)but-1-yn-1-yl)imidazo[1,2-a]pyrimidin-2-yl)phenyl)-2,4-dimethyloxazole-5-carboxamide 543a as a solid.

### Synthesis of Compound 543

The above solid 543a (0.44 g) was dissolved in MeOH and p-toluene sulfonic acid (0.15 g) was added and the resulting mixture was heated (60° C.) for 3 hours before it was poured into EtOAc and saturated NaHCO3. The organic phase was separated and the residue was purified over silica using CH₂Cl₂ and MeOH to give N-(4-fluoro-3-(6-(4-hydroxybut-1-yn-1-yl)imidazo[1,2-a]pyrimidin-2-yl)phenyl)-2,4-dimethyloxazole-5-carboxamide 543 as a solid. ¹H NMR (400 MHz, DMSO-d₆) δ 10.16 (s, 1H), 8.97 (d, J=2.4 Hz, 1H), 8.53 (dd, J=6.9, 2.8 Hz, 1H), 8.37 (d, J=2.4 Hz, 1H), 8.06 (d, J=4.1 Hz, 1H), 7.63 (ddd, J=8.9, 4.5, 2.8 Hz, 1H), 7.15 (dd, J=11.1, 8.9 Hz, 1H), 4.80 (t, J=5.6 Hz, 2H), 3.44 (td, J=6.7, 5.5 Hz, 2H), 2.44 (s, 3H), 2.31 (s, 3H). MS m/z 420.1 (M+1).

### Example 116

### Synthesis of N-(4-fluoro-3-(6-(4-morpholinobut-1-yn-1-yl)imidazo[1,2-a]pyrimidin-2-yl)phenyl)-2,4-dimethyloxazole-5-carboxamide (544)

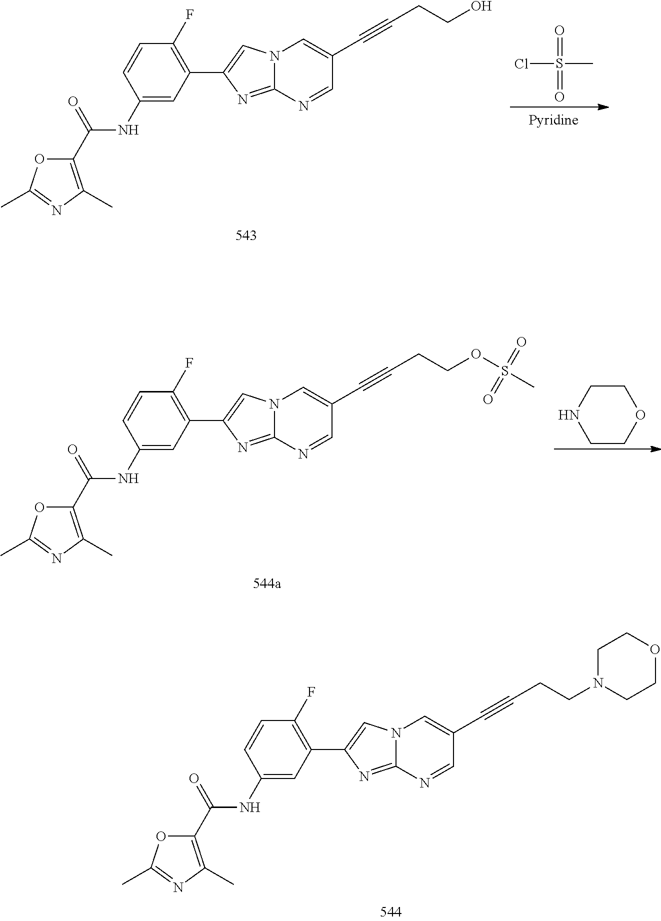

MsCl (33 mg) was added in one portion to a stirred solution of N-(4-fluoro-3-(6-(4-hydroxybut-1-yn-1-yl)imidazo[1,2-a]pyrimidin-2-yl)phenyl)-2,4-dimethyloxazole-5-carboxamide 543 (0.1 g) in pyridine. After 2 hours another 33 mg of MsCl was added and the resulting solution was stirred overnight. Morpholine (55 mg) was then added to the reaction mixture and heated to 50° C. for 5 hours. The reaction mixture was filtered and purified directly on reverse phase HPLC to give product 544. MS m/z 489.2 (M+1).

### Syntheses of Compound 545 and 546

Compounds 545 and 546 were synthesized following the protocol described above using piperidin-3-ol and 1-methylpiperazine respectively.

### Example 117

### Synthesis of N-(4-fluoro-3-(6-(2-methoxyethoxy)imidazo[1,2-a]pyrimidin-2-yl)phenyl)-2,4-dimethyloxazole-5-carboxamide (547)

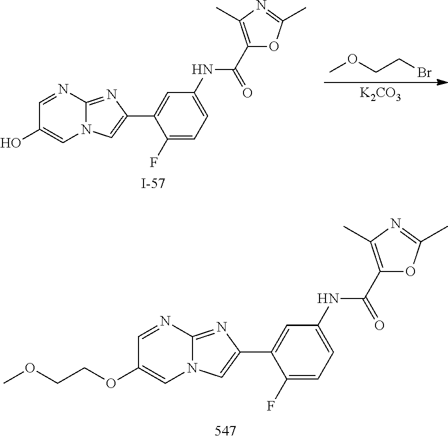

1-bromo-2-methoxyethane (53 mg) was added into a stirred mixture of N-(4-fluoro-3-(6-hydroxyimidazo[1,2-a]pyrimidin-2-yl)phenyl)-2,4-dimethyloxazole-5-carboxamide I-57 (70 mg) and K₂CO₃ (200 mg) in DMF. The resulting mixture was heated for 1 hour at 50° C. After cooling, the mixture was filtered and purified directly by HPLC to give N-(4-fluoro-3-(6-(2-methoxyethoxy)imidazo[1,2-a]pyrimidin-2-yl)phenyl)-2,4-dimethyloxazole-5-carboxamide 547. MS m/z 426.1 (M+1).

### Example 118

### Synthesis of methyl 2-(5-(2,4-dimethyloxazole-5-carboxamido)-2-fluorophenyl)imidazo[1,2-a]pyrimidine-6-carboxylate (548)

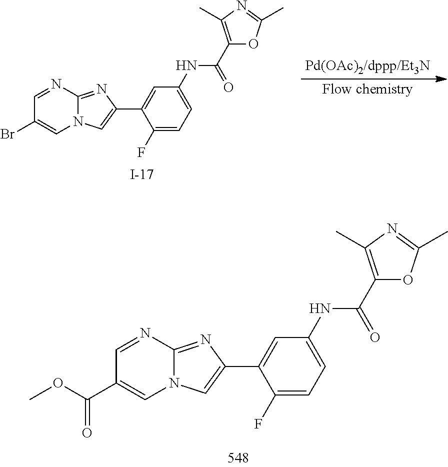

The reaction was performed using Flow chemistry with a Vapourtec R2C+/R4 (trip pressure set to 30 bar), tube-in-tube gas-liquid reactor, solution pressure set by 250 psi BPR, CO cylinder, two-stage regulator outlet gas pressure at 300 psi.

### Setup:

System solvent: MeOH/toluene (1:1)
Reagent A: 0.015M R1/Pd(OAc)₂/dppp/TEA (1:0.05:0.1:4, MeOH/toluene)
Flow rate A: 0.25 mL/m in
Reactor volume: 15 mL-tube-in-tube gas-liquid reactor
Reactor temperature: 130° C.
CO pressure: 300 psi
Back pressure regulator: 250 psi

### Synthesis of Compound 548

The system was purged with carbon monoxide by opening the supply and opening the outflow for 5 seconds. The outflow was closed, and the system gas pressure increased to 300 psi. The liquid system was purged of air by flushing with methanol/toluene in the absence of the back pressure regulator at 2.0 mL/min. The back pressure regulator (250 psi) was installed and the pump set to 0.4 mL/min. A solution of N-(3-(6-bromoimidazo[1,2-a]pyrimidin-2-yl)-4-fluorophenyl)-2,4-dimethyloxazole-5-carboxamide I-17/Pd(OAc)₂/dppp/Et₃N (1:0.05:0.1:4, 0.015M, MeOH/Toluene) was passed through the gas-liquid reactor at 130° C. for 60 minutes of residence time (0.25 mL/min). ¹H NMR (400 MHz, DMSO-d₆) δ 10.37 (s, 1H), 9.71 (d, J=2.4 Hz, 1H), 8.95 (d, J=2.4 Hz, 1H), 8.75 (dd, J=6.8, 2.8 Hz, 1H), 7.79-7.64 (m, 1H), 7.36 (dd, J=11.0, 8.9 Hz, 1H), 3.94 (s, 3H), 2.51 (s, 3H), 2.41 (s, 3H). MS m/z=410.0 (M+1).

### Example 119

### Synthesis of N-(4-fluoro-3-(6-(trimethylsilyl)imidazo[1,2-a]pyrimidin-2-yl)phenyl)-2,4-dimethyloxazole-5-carboxamide (549)

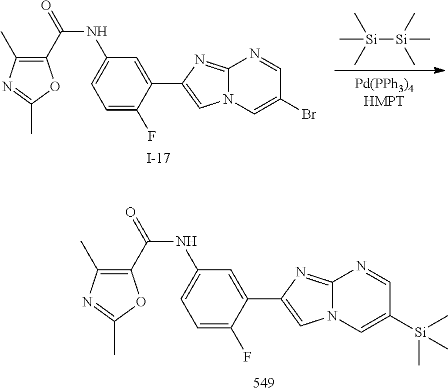

The mixture of 1-17 (200 mg, 0.46 mmol), 1,1,1,2,2,2-hexamethyldisilane (180 mg, 1.25 mmol) and tetrakis(triphenylphosphine)palladium (26 mg, 0.023 mmol) in HMPT (1 mL) was stirred at 120° C. under N₂ for 24 hours. The reaction mixture was cooled down, diluted with water (20 mL), extracted with 50% EtOAc/THF (20 mL×2). The combined organic layers were washed with brine (20 mL), dried with sodium sulfated, and concentrated. The residue was purified by HPLC to give 549 (63 mg, yield: 32%) as a white solid. ¹H NMR (400 MHz, DMSO-d₆) δ 10.31 (s, 1H), 9.02 (d, J=2.01 Hz, 1H), 8.73 (dd, J=6.90, 2.64 Hz, 1H), 8.63 (d, J=2.01 Hz, 1H), 8.26 (d, J=4.27 Hz, 1H), 7.78 (dd, J=7.53, 4.52 Hz, 1H), 7.28-7.37 (m, 1H), 2.48 (s, 3H), 2.40 (s, 3H), 0.36 (s, 9H). MS m/z=424.0 (M+1).

### Example 120

### Synthesis of isopropyl (2-(5-(5-chlorofuran-2-carboxamido)-2-fluorophenyl)imidazo[1,2-a]pyrimidin-6-yl)(methyl)carbamate (550) and isopropyl (2-(5-(5-chloro-N-methylfuran-2-carboxamido)-2-fluorophenyl)imidazo[1,2-a]pyrimidin-6-yl)(methyl)carbamate (551)

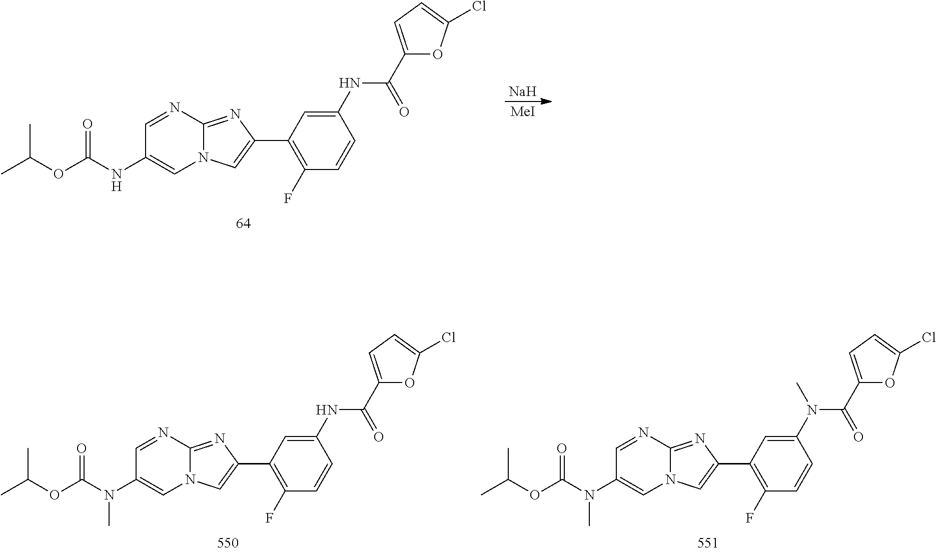

To a solution of isopropyl (2-(5-(5-chlorofuran-2-carboxamido)-2-fluorophenyl) imidazo[1,2-a]pyrimidin-6-yl)carbamate 64 (20 mg, 0.044 mmol) in DMF (2 mL) was added sodium hydride (2.1 mg, 0.052 mmol). The reaction was stirred at room temperature for 5 minutes then iodomethane (6 μl, 0.087 mmol) was added and stirring continued for 30 minutes. LC/MS showed two maJor product peaks. The crude products were purified by HPLC to afford isopropyl (2-(5-(5-chlorofuran-2-carboxamido)-2-fluorophenyl)imidazo[1,2-a]pyrimidin-6-yl)(methyl)carbamate 550 (3 mg) and isopropyl (2-(5-(5-chloro-N-methylfuran-2-carboxamido)-2-fluorophenyl)imidazo[1,2-a]pyrimidin-6-yl)(methyl)carbamate 551 (1.5 mg). Compound 550: ¹H NMR (400 MHz, CD₃OD) δ 9.08 (d, J=2.6 Hz, 1H), 8.83 (s, 1H), 8.31 (dt, J=4.7, 2.1 Hz, 2H), 7.81 (ddd, J=9.1, 4.4, 2.7 Hz, 1H), 7.34 (d, J=3.5 Hz, 1H), 7.30 (dd, J=10.8, 9.1 Hz, 1H), 6.57 (d, J=3.5 Hz, 1H), 5.00 (hept, J=6.0 Hz, 1H), 3.40 (s, 3H), 1.30 (d, J=6.4 Hz, 6H). Compound 551: ¹H NMR (400 MHz, CD₃OD) δ 9.02 (d, J=2.7 Hz, 1H), 8.73 (s, 1H), 8.33 (d, J=3.5 Hz, 1H), 8.14 (dt, J=6.7, 1.5 Hz, 1H), 7.45-7.33 (m, 2H), 6.34-6.20 (m, 2H), 5.02-4.93 (m, 1H), 3.47 (s, 3H), 3.38 (s, 3H), 1.28 (d, J=6.6 Hz, 6H).

### Syntheses of Compounds 552 to 559

Compounds 552, 553, 557 and 558 were synthesized following the protocol described above using compounds 61, 481 and I-33 as starting materials.

Compounds 554, 555 and 556 were synthesized using compound 61 and chloro(methoxy)methane, 1-chloro-2-methoxyethane and chloromethyl butyrate respectively as starting materials.

Compound 559 was synthesized using intermediate I-17 and 1,3-dichloropropane.

### Synthesis of Compound 560

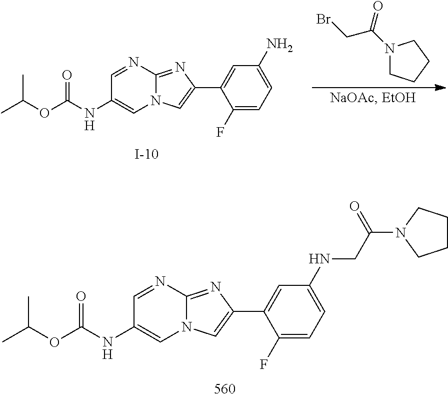

Compound 560 was synthesized using intermediate I-10 (20 mg), 2-bromo-1-(pyrrolidin-1-yl)ethanone (2 eq.) and NaOAc (3 eq.) as the base in ethanol (2 mL) treated at 100° C. overnight. LC/MS showed only 5% of product was formed. The mixture was purified by HPLC to give compound 560 (1 mg, 2% yield). MS m/z=441.1 (M+1).

### Example 121

### Synthesis of isopropyl (2-(2-chloro-5-(furan-2-carboxamido)phenyl)imidazo[1,2-a]pyrimidin-6-yl)(methyl)carbamate (561)

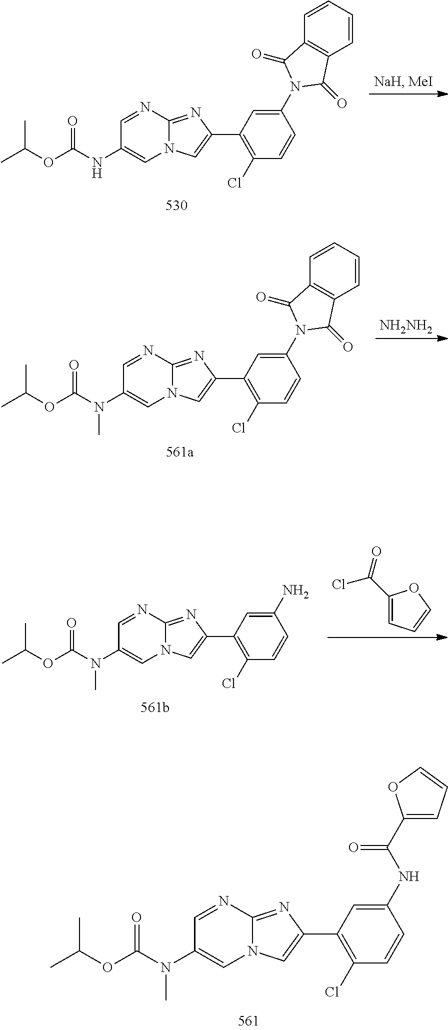

### Synthesis of Compound 561a

To a solution of isopropyl (2-(2-chloro-5-(1,3-dioxoisoindolin-2-yl)phenyl)imidazo[1,2-a]pyrimidin-6-yl)carbamate 530 (50 mg, 0.11 mmol) in DMF (2 mL) was added sodium hydride (7 mg, 0.16 mmol). The reaction was stirred at room temperature for 5 minutes then iodomethane (15 μl, 0.21 mmol) was added and stirring continued for 30 minutes. LC/MS showed one maJor product peak. The crude product was purified by HPLC to afford isopropyl (2-(2-chloro-5-(1,3-dioxoisoindolin-2-yl)phenyl)imidazo[1,2-a]pyrimidin-6-yl)(methyl)carbamate 561a (35 mg).

### Synthesis of Compound 561 b

To a solution of isopropyl (2-(2-chloro-5-(1,3-dioxoisoindolin-2-yl)phenyl)imidazo[1,2-a]pyrimidin-6-yl)(methyl)carbamate 561a (35 mg, 0.071 mmol) in ethanol (5 mL) was added hydrazine (12 mg, 0.36 mmol). The reaction was stirred at 100° C. overnight. The crude product was purified by HPLC to afford isopropyl (2-(5-amino-2-chlorophenyl)imidazo[1,2-a]pyrimidin-6-yl)(methyl)carbamate 561 b (14 mg).

### Synthesis of Compound 561

To a solution of isopropyl (2-(5-amino-2-chlorophenyl)imidazo[1,2-a]pyrimidin-6-yl)(methyl)carbamate 561b (14 mg, 0.039 mmol) in pyridine (3 mL) was added furan-2-carbonyl chloride (10 μl, 0.058 mmol). The reaction was stirred at room temperature overnight. The crude product was purified by HPLC to afford isopropyl (2-(2-chloro-5-(furan-2-carboxamido)phenyl)imidazo[1,2-a]pyrimidin-6-yl)(methyl)carbamate 561 (5 mg). ¹H NMR (400 MHz, CD₃OD) δ 9.18 (d, J=2.8 Hz, 1H), 8.96 (s, 1H), 8.48 (s, 1H), 8.32 (d, J=2.6 Hz, 1H), 7.84 (dd, J=8.8, 2.5 Hz, 1H), 7.78 (d, J=1.8 Hz, 1H), 7.61 (d, J=8.8 Hz, 1H), 7.32 (d, J=3.5 Hz, 1H), 6.67 (dd, J=3.5, 1.8 Hz, 1H), 5.01 (hept, J=6.3 Hz, 1H), 3.43 (s, 3H), 1.31 (d, J=6.3 Hz, 6H).

### Example 122

### Synthesis of N-(3-(6-(cyclopropylmethoxy)imidazo[1,2-a]pyrimidin-2-yl)-4-fluorophenyl)pyrrolidine-1-carboxamide (562)

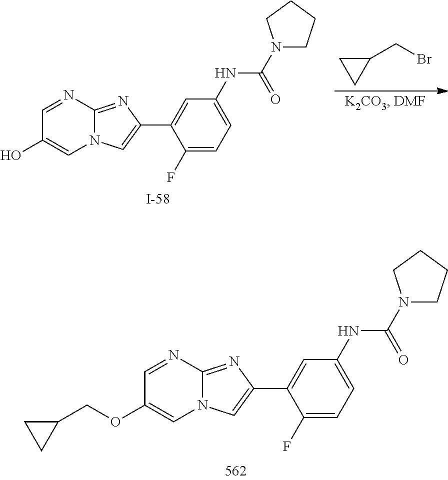

In a vial, intermediate I-58 (16.5 mg, 0.048 mmol), (bromomethyl)cyclopropane (7.83 mg, 0.058 mmol), and potassium carbonate (20.04 mg, 0.145 mmol) were taken up in DMF (2 mL) and heated to 75° C. overnight. The reaction was filtered and purified by HPLC to give the product 562 (2.9 mg, 6.82 μmol, 14% yield) as an off-white solid. ¹H NMR (400 MHz, CD₃OD) δ 8.50 (d, J=2.9 Hz, 1H), 8.44 (d, J=2.7 Hz, 1H), 8.10 (d, J=3.8 Hz, 1H), 7.94 (dd, J=6.6, 2.7 Hz, 1H), 7.56 (ddd, J=8.5, 4.3, 2.7 Hz, 1H), 7.14 (dd, J=11.0, 8.9 Hz, 1H), 3.90 (d, J=7.0 Hz, 2H), 3.54-3.45 (m, 4H), 1.99 (q, J=4.6, 2.9 Hz, 4H), 1.34 (td, J=5.4, 2.5 Hz, 1H), 0.73-0.62 (m, 2H), 0.46-0.37 (m, 2H).

### Synthesis of Compound 563

Compound 563 was synthesized following the protocol described above using 4-(2-chloroethyl)morpholine hydrochloride and adding sodium iodide as base.

### Example 123

### Synthesis of Compounds 564

Compound 564 was synthesized according to the protocol described in Example 90 using intermediate I-59.

### Example 124

### Synthesis of N-(4-fluoro-3-(6-(4-methylpiperazin-1-yl)imidazo[1,2-a]pyrimidin-2-yl)phenyl)-2,4-dimethyloxazole-5-carboxamide (565)

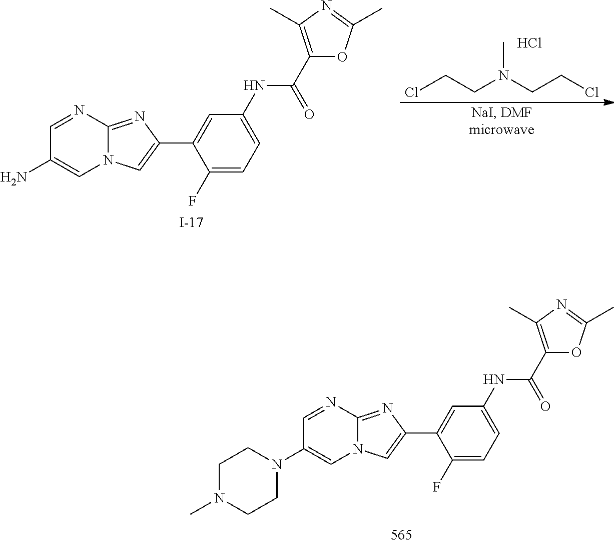

In a microwave vial, intermediate I-17 (20 mg, 0.050 mmol), mechlorethamine hydrochloride (16.25 mg, 0.084 mmol), sodium iodide (22.33 mg, 0.149 mmol), and potassium carbonate (27.4 mg, 0.199 mmol) were taken up in DMF (1 mL) and irradiated for 4 hours at 120° C. The reaction was filtered and purified by HPLC to give the product 565 (3.2 mg, 4.06 μmol, 8.17% yield) as an orange solid. MS m/z=450.2 (M+1)

### Example 125

### Synthesis of N-(4-fluoro-3-(6-(2,2,2-trifluoroacetamido)imidazo[1,2-a]pyrimidin-2-yl)phenyl)pyrrolidine-1-carboxamide (566)

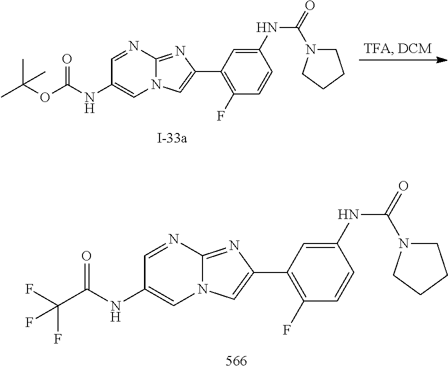

In a 50 mL round bottom flask containing tert-butyl (2-(2-fluoro-5-(pyrrolidine-1-carboxamido)phenyl)imidazo[1,2-a]pyrimidin-6-yl)carbamate I-33a (0.295 mmol, 130 mg) dissolved in DCM (5 mL), trifluoroacetic acid (3 mL) was added and the solution was stirred at room temperature for 16 hours. The reaction mixture was concentrated in vacuo to give an oily material which was shook at room temperature for 96 hours; the oil obtained was diluted with DCM and Amberlyst resin A21 (1 g) was added. After filtration of the resin, the filtrate was concentrated in vacuo and purified by HPLC which allowed the isolation of product 566 as a white solid (25 mg, 20%). ¹H NMR (400 MHz, DMSO-d₆) δ 11.80 (brs, 1H), 9.55 (d, J=2.57 Hz, 1H), 8.67 (d, J=2.69 Hz, 1H), 8.41 (d, J=4.16 Hz, 1H), 8.37 (dd, J=6.85, 2.81 Hz, 1H), 8.34 (s, 1H), 7.65 (ddd, J=8.89, 4.49, 2.87 Hz, 1H), 7.19 (dd, J=11.06, 8.99 Hz, 1H), 3.39 (t, J=6.60 Hz, 4H), 1.83-1.89 (m, 4H). MS m/z=437.9 (M+1)

### Example 126

### Synthesis of N-(2-(5-acetamido-2-fluorophenyl)imidazo[1,2-a]pyrimidin-6-yl)acetamide (567)

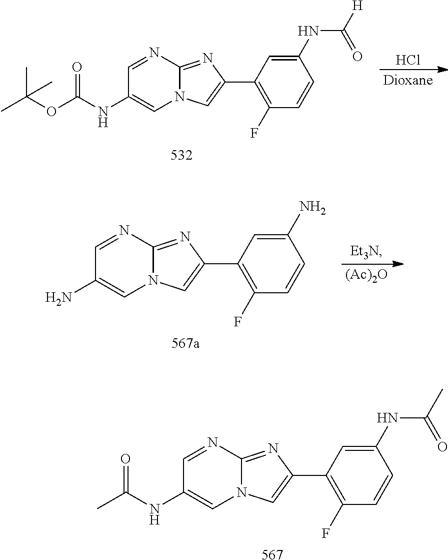

### Synthesis of 2-(5-amino-2-fluorophenyl)imidazo[1,2-a]pyrimidin-6-amine 567a

In a 10 mL vial round bottom flask containing tert-butyl (2-(2-fluoro-5-formamidophenyl)imidazo[1,2-a]pyrimidin-6-yl)carbamate 532 (0.032 mmol, 12 mg) dissolved in 2 mL of 1,4-dioxane, hydrochloric acid (37%, 1 mL) was added. The reaction mixture was stirred overnight at room temperature. The crude mixture was concentrated in vacuo to give product 567a as a white solid (9 mg, 88%). ¹H NMR (400 MHz, DMSO-d₆) δ 8.54 (d, J=2.69 Hz, 1H), 8.43 (d, J=3.42 Hz, 1H), 8.35 (d, J=2.69 Hz, 1H), 7.83 (brs, 1H), 7.40-7.47 (m, 1H), 7.28 (br. s., 1H). MS m/z=244.8 (M+1)

### Synthesis of Compound 567

2-(5-amino-2-fluorophenyl)imidazo[1,2-a]pyrimidin-6-amine 567a (9 mg, 0.037 mmol) was dissolved in DCM and triethylamine (0.259 mmol, 36 μl) was added followed by acetic anhydride (0.222 mmol, 22 μl). The reaction mixture was stirred at room temperature for 4 hours. Upon completion, the solution obtained was concentrated in vacuo. The residue was triturated in water. Filtration over Bchner gave product 567 as a pale brown solid (5 mg, 40%). ¹H NMR (400 MHz, DMSO-d₆) δ 10.38 (s, 1H), 10.10 (s, 1H), 9.54 (d, J=2.57 Hz, 1H), 8.49 (d, J=2.57 Hz, 1H), 8.42 (dd, J=6.79, 2.63 Hz, 1H), 8.38 (d, J=4.28 Hz, 1H), 7.64-7.70 (m, 1H), 7.25 (dd, J=10.94, 8.99 Hz, 1H), 2.12 (s, 3H), 2.06 (s, 3H). MS m/z=328.9 (M+1).

### Example 127

### Synthesis of N-(5-(6-aminoimidazo[1,2-a]pyrimidin-2-yl)-2-fluorophenyl)pyrrolidine-1-carboxamide (568)

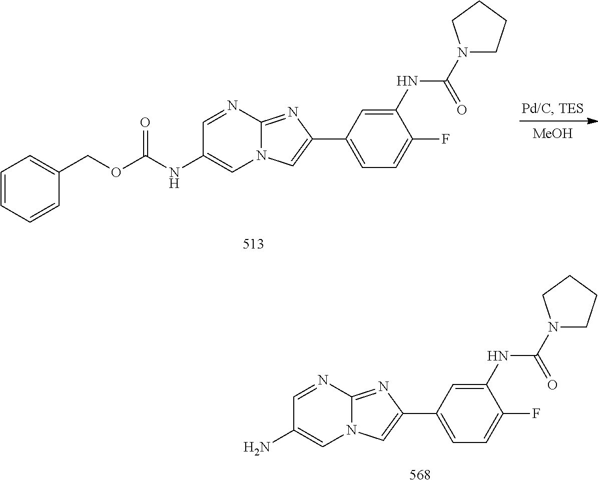 [SMILES: "MeOH" ; O=C(Nc1cnc2nc(-c3ccc(F)c(NC(=O)N4CCCC4)c3)cn2c1)OCc1ccccc1 ; Nc1cnc2nc(-c3ccc(F)c(NC(=O)N4CCCC4)c3)cn2c1]

In a 50 mL round bottom flask containing Benzyl (2-(4-fluoro-3-(pyrrolidine-1-carboxamido) phenyl)imidazo[1,2-a]pyrimidin-6-yl)carbamate 513 (0.053 mmol, 25 mg) dissolved in MeOH, Pd/C (20% by weight, 5 mg) was added. The reaction mixture was degassed and triethylsilane (0.790 mmol, 0.126 mL) was added. The mixture was stirred at room temperature for 1 h 30. The reaction mixture was filtered over celite and the filtrate was adsorbed onto silica and purified by flash column chromatography (DCM/MeOH) to afford product 568 as a white solid (5 mg, 26%). ¹H NMR (400 MHz, DMSO-d₆) δ 8.18 (d, J=2.81 Hz, 1H), 8.09-8.13 (m, 2H), 8.04 (d, J=2.81 Hz, 1H), 7.87 (s, 1H), 7.61 (ddd, J=8.47, 4.74, 2.20 Hz, 1H), 7.23 (dd, J=10.51, 8.44 Hz, 1H), 5.17 (s, 2H), 3.40 (s, 4H), 1.88 (t, J=6.54 Hz, 4H). MS m/z=341.1

### Example 128

### Synthesis of N-(5-(6-aminoimidazo[1,2-a]pyrimidin-2-yl)-2-fluorophenyl)pyrrolidine-1-carboxamide (569)

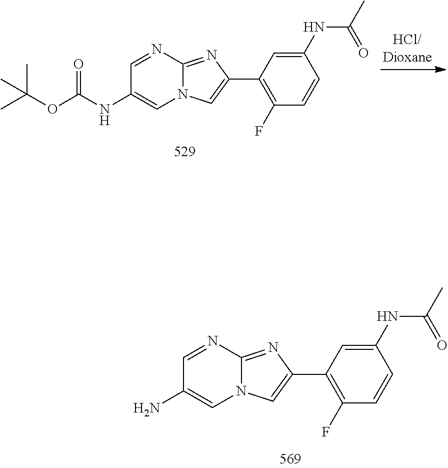 [SMILES: CC(=O)Nc1ccc(F)c(-c2cn3cc(NC(=O)OC(C)(C)C)cnc3n2)c1 ; CC(=O)Nc1ccc(F)c(-c2cn3cc(N)cnc3n2)c1]

In a 25 mL round bottom flask containing tert-butyl (2-(5-acetamido-2-fluorophenyl)imidazo[1,2-a]pyrimidin-6-yl)carbamate 529 (60 mg, 0.156 mmol), dissolved in 1,4-dioxane (5 mL), aqueous hydrochloric acid 37% (0.3 mL, 3.65 mmol) was added and the solution was stirred at room temperature for 40 minutes. The reaction mixture was treated with Amberlyst A21 to remove HCl. After filtration, the crude mixture was concentrated in vacuo to give product 569 as a pale brown solid (44 mg, 97%). ¹H NMR (400 MHz, DMSO-d₆) δ 10.15 (brs, 1H), 8.38 (dd, J=6.85, 2.69 Hz, 1H), 8.24 (d, J=2.57 Hz, 1H), 8.09 (dd, J=8.31, 3.06 Hz, 2H), 7.64 (ddd, J=8.80, 4.46, 2.87 Hz, 1H), 7.21 (dd, J=11.07, 8.86 Hz, 1H), 5.24 (s, 2H), 2.06 (s, 3H). MS m/z=286.9 (M+1).

### Example 129

### Synthesis of N-(4-chloro-3-(6-(1,2,3,6-tetrahydropyridin-4-yl)imidazo[1,2-a]pyrimidin-2-yl)phenyl)pyrrolidine-1-carboxamide (570)

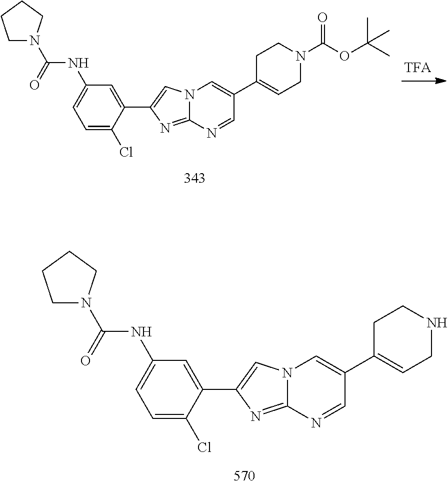 [SMILES: CC(C)(C)OC(=O)N1CC=C(c2cnc3nc(-c4cc(NC(=O)N5CCCC5)ccc4Cl)cn3c2)CC1 ; O=C(Nc1ccc(Cl)c(-c2cn3cc(C4=CCNCC4)cnc3n2)c1)N1CCCC1]

In a vial, compound 343 (7.3 mg, 0.014 mmol) was taken up in TFA (1 mL) and stirred at room temperature for 10 minutes, then the solvent was evaporated in vacuo. The residue was taken up in MeOH, neutralized (StratoSpheres SPE PL-HCO₃), and concentrated in vacuo to give the product 570 (5.8 mg, 0.014 mmol, 98% yield) as an off-white solid. ¹H NMR (400 MHz, DMSO-d₆): 8.98 (d, J=2.5 Hz, 1H), 8.86 (d, J=2.5 Hz, 1H), 8.51 (s, 1H), 8.48-8.39 (m, 2H), 7.72 (dt, J=8.8, 2.3 Hz, 1H), 7.39 (d, J=8.8 Hz, 1H), 6.50 (s, 1H), 4.05 (s, 1H), 3.39 (q, J=6.3, 4.5 Hz, 4H), 3.32 (s, 2H), 2.94 (t, J=5.6 Hz, 2H), 2.39-2.27 (m, 2H), 1.93-1.78 (m, 4H).

### Synthesis of Compounds 571 to 574

Compounds 571 to 574 were synthesized following the protocol described above using compounds 293, 384, 364 and 345 as starting materials.

### Example 130

### Synthesis of N-(4-fluoro-3-(6-(2,2,2-trifluoroethyl)imidazo[1,2-a]pyrimidin-2-yl)phenyl)-2,4-dimethyloxazole-5-carboxamide (575)

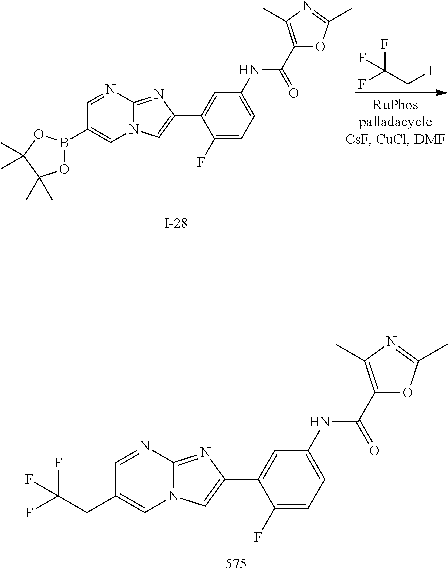

In a vial, intermediate I-28 (50 mg, 0.105 mmol), 1,1,1-trifluoro-2-iodoethane (0.021 mL, 0.210 mmol), cesium fluoride (47.7 mg, 0.314 mmol), water (0.015 mL, 0.838 mmol), CuCl (10.37 mg, 0.105 mmol), and RuPhos palladacycle (4.28 mg, 5.24 μmol) were taken up in DMF (2 mL) and the resulting mixture was sparged with argon and heated to 75° C. overnight. The reaction was filtered and purified by HPLC. to give the product 575 (1.2 mg, 2% yield) as an off-white solid. ¹H NMR (400 MHz, DMSO-d₆) δ 10.34 (s, 1H), 9.08 (d, J=2.3 Hz, 1H), 8.73 (dd, J=6.8, 2.8 Hz, 1H), 8.58 (d, J=2.3 Hz, 1H), 8.38 (d, J=4.3 Hz, 1H), 7.80 (ddd, J=9.0, 4.5, 2.8 Hz, 1H), 7.33 (dd, J=10.9, 9.0 Hz, 1H), 3.86 (q, J=11.3 Hz, 2H), 2.51 (s, 3H), 2.40 (s, 3H). MS m/z=434.1 (M+1)

### Example 131

### Synthesis of 5-fluoro-N-(4-fluoro-3-(6-phenylimidazo[1,2-a]pyrimidin-2-yl)phenyl)furan-2-carboxamide (576)

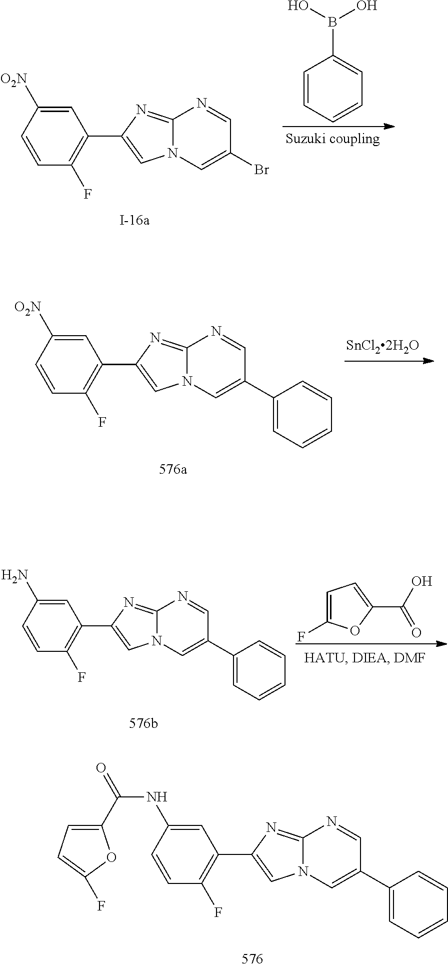

Compound 575 was synthesized by Suzuki coupling using I-16a and phenyl boronic acid following the procedure described for the synthesis of compound I-13 in Example 7, followed by the reduction of the nitro group with tin chloride using the procedure described for the synthesis of compound I-4, and finally an amide coupling with 5-fluorofuran-2-carboxylic acid I-60 using the amide coupling procedure used for the synthesis of I-17.

### Example 132

### Synthesis of N-(3-(6-aminoimidazo[1,2-a]pyrimidin-2-yl)-4-chlorophenyl)-5-fluorofuran-2-carboxamide (577)

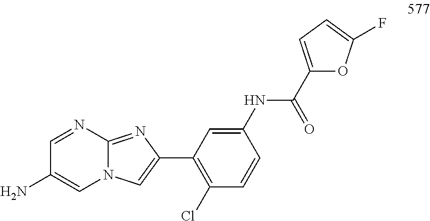 [SMILES: Nc1cnc2nc(-c3cc(NC(=O)c4ccc(F)o4)ccc3Cl)cn2c1]

Compound 577 was synthesized in a similar fashion as compound I-30 using chlorinated intermediate I-2 instead of I-10b.

### Example 133

### Synthesis of N-(3-(64(1-(dimethylamino)propan-2-yl)amino)imidazo[1,2-a]pyrimidin-2-yl)-4-fluorophenyl)-2,4-dimethyloxazole-5-carboxamide (578)

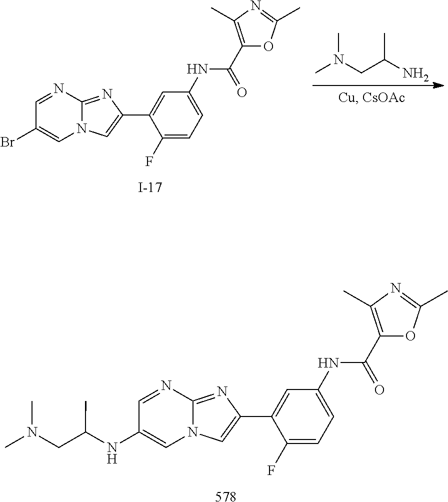

A dry vial was charged with copper powder (0.5 mg, 0.009 mmol), CsOAc (36 mg, 0.186 mmol), I-17 (40 mg, 0.093 mmol) and N1,N1-dimethylpropane-1,2-diamine (0.279 mmol) followed by dry DMSO (1 mL). The vial was sealed and the mixture was heated to 90° C. for 2 hours. AcOEt was added and the mixture was filtered through silica gel, concentrated and purified by HPLC. MS m/z=452.2 (M+1)

### Example 134

### Syntheses of Compounds 579

Compound 579 was synthesized by following the urea coupling reaction described in Example 96 using intermediates I-56, I-35 and I-61 respectively.

### Example 135

### Synthesis of Compound 580

Compound 580 was synthesized following the protocol described in Example 128 using compound 579 as starting material.

### Example 136

### Synthesis of N-(4-fluoro-3-(6-vinylimidazo[1,2-a]pyrimidin-2-yl)phenyl)-2,4-dimethyloxazole-5-carboxamide (581)

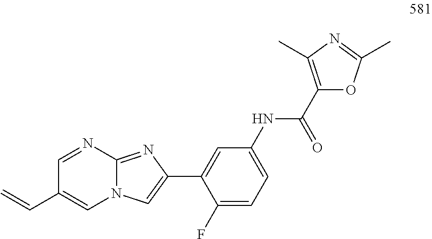 [SMILES: C=Cc1cnc2nc(-c3cc(NC(=O)c4oc(C)nc4C)ccc3F)cn2c1]

Compound 581 was synthesized in a similar fashion as compound I-47a in Example 32 using intermediate I-17 as starting material. ¹H NMR (400 MHz, DMSO-d₆) δ 10.32 (s, 1H), 9.07 (d, J=2.26 Hz, 1H), 8.90 (d, J=2.26 Hz, 1H), 8.73 (dd, J=6.78, 2.51 Hz, 1H), 8.26 (d, J=4.27 Hz, 1H), 7.80 (dd, J=7.53, 4.27 Hz, 1H), 7.33 (dd, J=11.04, 9.03 Hz, 1H), 6.78 (dd, J=17.69, 11.17 Hz, 1H), 6.05 (d, J=17.82 Hz, 1H), 5.45 (d, J=11.29 Hz, 1H), 2.48 (s, 3H), 2.41 (s, 3H).

### Example 137

### Synthesis of N-(3-(6-(ethylamino)imidazo[1,2-a]pyrimidin-2-yl)-4-fluorophenyl)-2,4-dimethyloxazole-5-carboxamide (582)

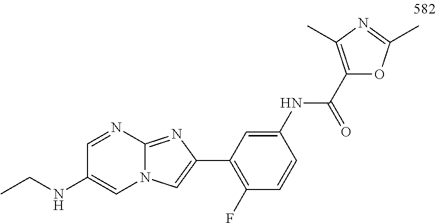 [SMILES: CCNc1cnc2nc(-c3cc(NC(=O)c4oc(C)nc4C)ccc3F)cn2c1]

Compound 582 was synthesized in a similar fashion as compound 212 using ethyl iodide instead of methyl iodide for the synthesis of intermediate I-50. ¹H NMR (400 MHz, DMSO-d₆) δ 10.19-10.38 (m, 1H), 8.52-8.76 (m, 1H), 8.19-8.41 (m, 1H), 7.92-8.16 (m, 2H), 7.63-7.83 (m, 1H), 7.17-7.38 (m, 1H), 5.85 (brs, 1H), 2.87-3.09 (m, 2H), 2.46-2.50 (m, 3H), 2.40 (s, 3H), 1.23 (s, 3H). MS m/z=395.2 (M+1).

### Example 138

### Synthesis of 3-(3-(6-(dimethylamino)imidazo[1,2-a]pyrimidin-2-yl)-4-fluorophenyl)-1,1-dimethylurea (583)

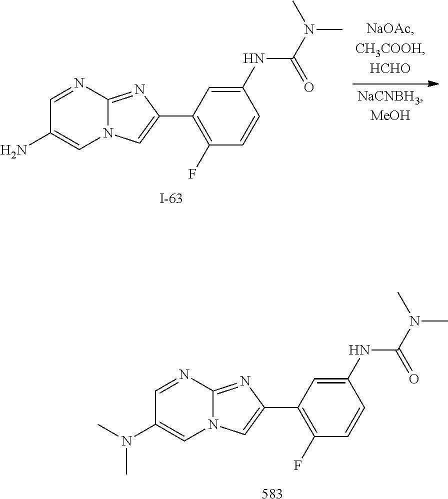

Compound 583 was synthesized in a similar fashion as compound 466 in Example 91 using intermediate I-63 as starting material. ¹H NMR (400 MHz, DMSO-d₆) δ 8.60 (brs, 1H), 8.46 (brs, 1H), 8.30 (brs, 2H), 8.09 (brs, 1H), 7.54 (m., 1H), 7.17 (t, J=9.54 Hz, 1H), 2.95 (brs, 6H), 2.88 (brs, 6H). MS m/z=343.1 (M+1).

### Example 139

### Synthesis of 4-fluoro-3-(6-isopropylimidazo[1,2-a]pyrimidin-2-yl)aniline (584)

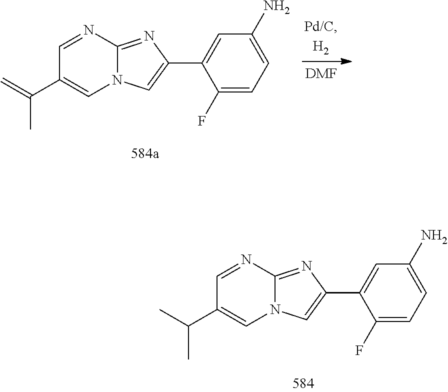

Compound 584a was synthesized in a similar fashion as compound I-16 in Example 10 using intermediate I-52 as starting material.

Compound 584 was prepared from 584a following the same procedure used for the hydrogenation of 411a to give 411. ¹H NMR (400 MHz, DMSO-d₆) δ 8.84 (d, J=2.5 Hz, 1H), 8.56 (d, J=2.5 Hz, 1H), 8.11 (d, J=4.1 Hz, 1H), 7.50 (dd, J=6.5, 3.1 Hz, 1H), 6.96 (dd, J=11.4, 8.7 Hz, 1H), 6.53 (dq, J=8.8, 3.0 Hz, 1H), 3.01 (p, J=6.9 Hz, 1H), 1.29 (d, J=6.8 Hz, 6H). ¹⁹F NMR (376 MHz, DMSO-d₆) δ−130.47.

### Example 140

### Synthesis of N-(4-fluoro-3-(6-(5-(2-hydroxyethyl)pyridin-2-yl)imidazo[1,2-a]pyrimidin-2-yl)phenyl)-2,4-dimethyloxazole-5-carboxamide (585)

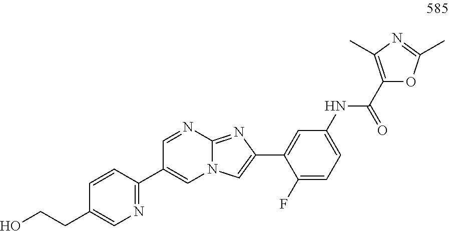 [SMILES: Cc1nc(C)c(C(=O)Nc2ccc(F)c(-c3cn4cc(-c5ccc(CCO)cn5)cnc4n3)c2)o1]

Compound 585 was synthesized in a similar fashion as compound 430 in Example 86 using 2-(6-bromopyridin-3-yl)ethanol as starting material. ¹H NMR (400 MHz, DMSO-d₆) δ 10.36 (s, 1H), 9.69 (d, J=2.4 Hz, 1H), 9.28 (d, J=2.4 Hz, 1H), 8.76 (dd, J=7.0, 2.7 Hz, 1H), 8.59 (d, J=2.3 Hz, 1H), 8.40 (d, J=4.1 Hz, 1H), 8.00 (d, J=8.1 Hz, 1H), 7.89-7.76 (m, 2H), 7.34 (dd, J=11.0, 9.0 Hz, 1H), 4.76 (t, J=5.1 Hz, 1H), 3.67 (td, J=6.6, 5.1 Hz, 2H), 2.81 (t, J=6.6 Hz, 2H), 2.51 (s, 3H), 2.41 (s, 3H).

### Example 141

### Synthesis of 3-(3-(6-bromoimidazo[1,2-a]pyrimidin-2-yl)-4-fluorophenyl)-1-methyl-1-(2,2,2-trifluoroethyl)urea (586)

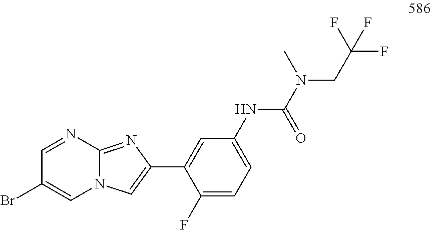 [SMILES: CN(CC(F)(F)F)C(=O)Nc1ccc(F)c(-c2cn3cc(Br)cnc3n2)c1]

Compound 586 was synthesized in a similar fashion as compound I-21 in Example 14 using 2,2,2-trifluoro-N-methylethanamine hydrochloride as starting material. ¹H NMR (400 MHz, DMSO-d₆) δ 9.36 (s, 1H), 8.64 (s, 1H), 8.50 (s, 1H), 8.40 (dd, J=6.78, 2.76 Hz, 1H), 8.23 (d, J=4.02 Hz, 1H), 7.61-7.74 (m, 1H), 7.24 (dd, J=11.04, 9.03 Hz, 1H), 5.27-5.50 (m, 1H), 3.39-3.83 (m, 4H), 1.98-2.28 (m, 2H). MS m/z=446.0 (M+1), 448.0 (M+3).

### Example 142

### Synthesis of 3-fluoro-N-(4-fluoro-3-(6-(trifluoromethyl)imidazo[1,2-a]pyrimidin-2-yl)phenyl)azetidine-1-carboxamide (587)

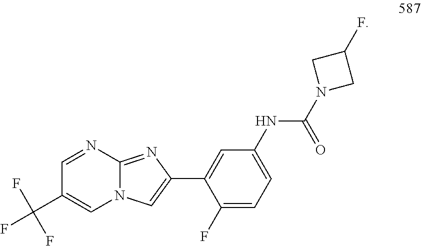 [SMILES: O=C(Nc1ccc(F)c(-c2cn3cc(C(F)(F)F)cnc3n2)c1)N1CC(F)C1]

Compound 587 was synthesized in a similar fashion as compound I-21 using intermediate I-36 as starting material. ¹H NMR (400 MHz, DMSO-d₆-): 9.65 (s, 1H), 8.89 (d, J=2.26 Hz, 1H), 8.85 (s, 1H), 8.34-8.43 (m, 2H), 7.63-7.74 (m, 1H), 7.26 (dd, J=10.79, 9.03 Hz, 1H), 5.26-5.52 (m, 1H), 4.21-4.41 (m, 2H), 3.91-4.11 (m, 2H). MS m/z=398.0 (M+1)

### Example 143

### Syntheses of Compounds 588 and 589

Compounds 588 and 589 were synthesized according to the protocol described in Example 86 and the appropriate aryl bromide.

### Example 144

### Synthesis of N-(4-fluoro-3-(6-isobutylimidazo[1,2-a]pyrimidin-2-yl)phenyl)-2,4-dimethyloxazole-5-carboxamide (590)

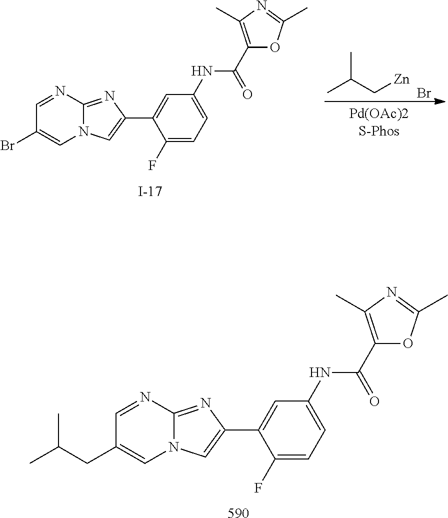

Intermediate I-17 (20 mg, 1 eq.), diacetoxypalladium (2 mg, 0.1 eq.) and S-Phos (2-Dicyclohexylphosphino-2′,6′-dimethoxybiphenyl) (6 mg, 0.2 eq.) were taken up in THF (2 mL). Isobutylzinc(II) bromide(0.7 mL, 5 eq.) was added and the solution was sparged with N₂ for 5 minutes and stirred overnight at 80° C. The reaction mixture was filtered and purified by HPLC to give the compound 590 (10 mg, 35%). ¹H NMR (400 MHz, CD₃OD) δ 8.70 (d, J=2.3 Hz, 1H), 8.49 (d, J=2.4 Hz, 1H), 8.25 (dd, J=6.6, 2.7 Hz, 1H), 8.18 (d, J=3.9 Hz, 1H), 7.84 (ddd, J=8.9, 4.5, 2.8 Hz, 1H), 7.24 (dd, J=10.9, 8.9 Hz, 1H), 2.59 (d, J=7.2 Hz, 2H), 2.56 (s, 3H), 2.47 (s, 3H), 1.97 (dd, J=13.6, 6.9 Hz, 1H), 1.00 (d, J=6.6 Hz, 6H).

Compound 591 was synthesized following the protocol described above using neopentylzinc bromide as starting materials.

### Example 145

### Synthesis of Compound 592

Compound 592 was synthesized according to the protocol described in Example 90 using intermediate I-64.

### Example 146

### Synthesis of Compound 593

Compound 593 was synthesized according to the protocol described in Example 91 using intermediates I-64.

### Example 147

### Synthesis of 4-fluoro-3-(6-(3-methylpyridin-2-yl)imidazo[1,2-a]pyrimidin-2-yl)aniline (594)

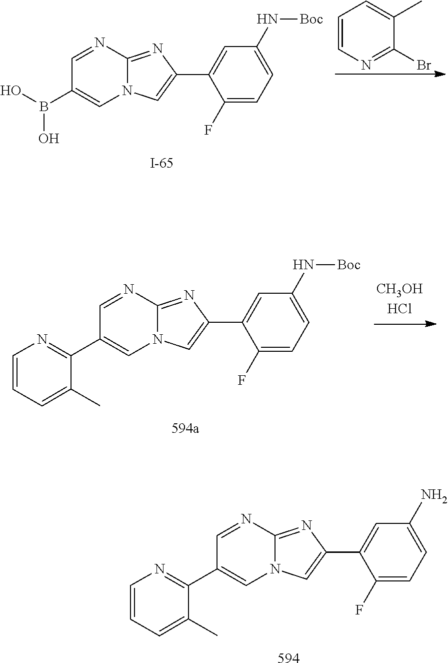

### Synthesis of 594a

To a solution of intermediate I-65 (820 mg, 2.2 mmol), 2-bromo-3-methylpyridine (758.1 mg, 4.4 mmol) and potassium carbonate (609 mg, 4.4 mmol) in 1,4-dioxane/water=10/1 (11 mL) was added tetrakis(triphenyl phosphine) palladium (254.6 mg, 0.22 mmol) under N₂, the reaction was stirred at 100° C. for 15 hours. Then the mixture was concentrated and purified by silica gel chromatography (DCM/THF=1) to afford compound 594a (0.9 g, crude) in 80% purity as a brown oil. MS m/z=420.1 (M+1)

### Synthesis of Compound 594

A solution of compound 594a (0.9 g, 2.15 mmol) in MeOH—HCl (20 mL) was stirred at room temperature for 3 hours. Then the mixture was concentrated and basified with a solution of sodium bicarbonate to pH=8. The mixture was filtered and the filter cake was washed with water (10 mL×2) and methanol (10 mL), collected and dried in vacuo to give 594 (130 mg, yield, 24%) as green solid. ¹H NMR (DMSO-d₆) δ 9.29 (d, J=2.51 Hz, 1H), 8.82 (d, J=2.51 Hz, 1H), 8.58 (d, J=4.02 Hz, 1H), 8.27 (d, J=4.02 Hz, 1H), 7.82 (d, J=7.53 Hz, 1H), 7.54-7.57 (m, 1H), 7.39-7.42 (m, 1H), 6.98-7.03 (m, 1H), 6.55-6.58 (m, 1H), 5.15 (s, 2H) 2.49 (s, 3H). MS m/z=320.0 (M+1).

### Example 148

### Synthesis of 4-fluoro-3-(6-(3-methylpyridin-2-yl)imidazo[1,2-a]pyrimidin-2-yl)aniline (595)

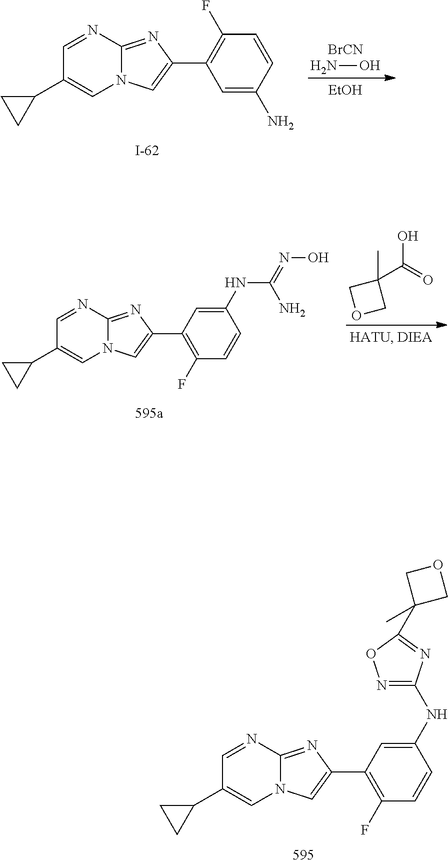

### Synthesis of Compound 595a

BrCN (0.043 g) was added in one portion to a stirred solution of 3-(6-cyclopropylimidazo[1,2-a]pyrimidin-2-yl)-4-fluoroaniline I-62 (0.1 g) and NaOAc (0.1 g) in MeOH. After 3 hours, the solvent was evaporated and the mixture resuspended in dry EtOH. NH₂OH (2 mL, 50% in water) was added to the mixture and the resulting solution was stirred for 3 hours. The solvent was evaporated and the product 595a was used without further purification.

### Synthesis of Compound 595

HATU (70 mg) was added in one portion to 3-methyloxetane-3-carboxylic acid (20 mg) and iPr₂NEt (22 mg) in dry DMF. After 20 minutes, the hydroxy guanidine 595a (50 mg) was added to the reaction mixture. After 30 minutes, the mixture was heated to 100° C. for another 3 hours. The mixture was filtered and purified directly on reverse phase HPLC to give N-(3-(6-cyclopropylimidazo[1,2-a]pyrimidin-2-yl)-4-fluorophenyl)-5-(3-methyloxetan-3-yl)-1,2,4-oxadiazol-3-amine 595. ¹H NMR (400 MHz, CD₃OD) δ 8.92-8.78 (m, 2H), 8.32 (d, J=2.6 Hz, 1H), 8.04 (dd, J=6.3, 2.8 Hz, 1H), 7.60 (ddd, J=9.0, 4.2, 2.7 Hz, 1H), 7.33 (dd, J=10.6, 9.0 Hz, 1H), 5.07 (d, J=6.0 Hz, 2H), 4.65 (d, J=6.0 Hz, 2H), 2.28-2.05 (m, 1H), 1.84 (s, 3H), 1.30-1.12 (m, 2H), 1.03-0.85 (m, 2H). MS m/z=407.2 (M+1).

### Synthesis of Compound 596

Compound 596 was synthesized following the protocol described above using 3-hydroxy-2,2-dimethylpropanoic acid as starting materials.

### Example 149

### Syntheses of Compounds 597 to 603

Compounds 597 to 603 were synthesized by following the amide coupling reaction described in Example 51 using the intermediate I-62 and the corresponding carboxylic acids.

### Example 150

### Syntheses of Compounds 604 to 606

Compounds 604 to 606 were synthesized following the urea coupling reaction described in Example 71 using the intermediate I-62 and the appropriate amines.

### Example 151

### Synthesis of Compound 607

Compound 607 was synthesized by following the amide coupling reaction described in Example 51 using the intermediate I-58b and 2,4-dimethyloxazole-5-carboxylic acid.

### Example 152

### Synthesis of N-(4-fluoro-3-(6-isopropoxyimidazo[1,2-a]pyrimidin-2-yl)phenyl)-2,4-dimethyloxazole-5-carboxamide (608)

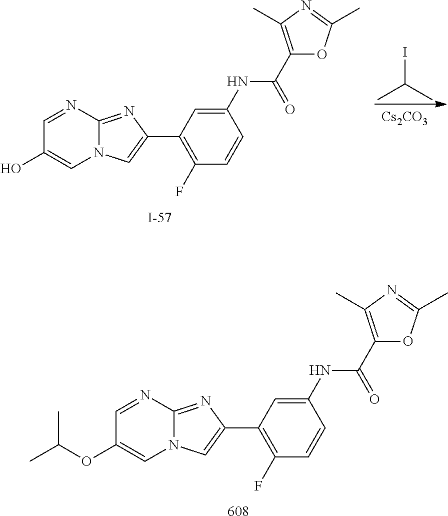

2 lodopropane (40 mg) was added to a stirred mixture of 1-57 (50 mg) and Cs₂CO₃ (220 mg) in DMF. The resulting solution was heated at 50° C. overnight. The mixture was filtered before subjected to reverse phase HPLC purification to give N-(4-fluoro-3-(6-isopropoxyimidazo[1,2-a]pyrimidin-2-yl)phenyl)-2,4-dimethyloxazole-5-carboxamide 608. ¹H NMR (400 MHz, DMSO-d₆) δ 10.34 (s, 1H), 8.81 (d, J=2.9 Hz, 1H), 8.68 (dd, J=6.9, 2.7 Hz, 1H), 8.47 (d, J=2.9 Hz, 1H), 8.20 (d, J=4.2 Hz, 1H), 7.78 (ddd, J=8.9, 4.5, 2.8 Hz, 1H), 7.33 (dd, J=11.0, 8.9 Hz, 1H), 4.55 (p, J=6.0 Hz, 1H), 2.40 (s, 3H), 2.39 (s, 3H), 1.35 (dd, J=6.0, 2.8 Hz, 6H). MS m/z=410.2 (M+1).

Compound 609 to 611 were synthesized following the protocol described above using methyl 2-bromoacetate, (bromomethyl)cyclopropane and 2-(bromomethyl)pyridine hydrobromide respectively as starting materials.

Compound 612 was prepared using 2 equivalents of 2-iodopropane.

### Example 153

### Synthesis of N-(3-(6-(cyclobutylmethyl)imidazo[1,2-a]pyrimidin-2-yl)-4-fluorophenyl)-2,4-dimethyloxazole-5-carboxamide (613)

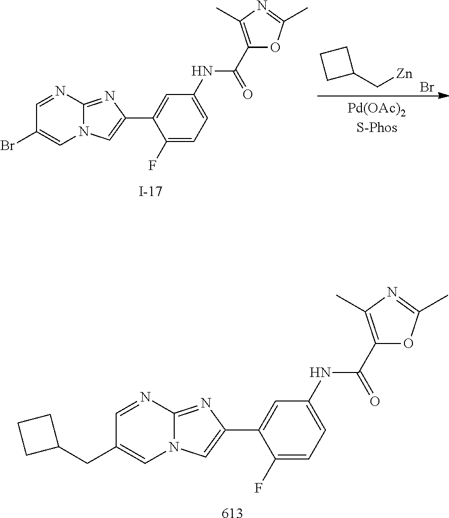

Intermediate I-17 (30 mg, 1 eq.), diacetoxypalladium (2 mg, 0.1 eq.) and S-Phos (2-Dicyclohexylphosphino-2′,6′-dimethoxybiphenyl) (6 mg, 0.2 eq.) were taken up in THF (3 mL). To the solution was added (cyclobutylmethypzinc(II) bromide (0.7 mL, 5 eq.) and the mixture was sparged with N₂ for 5 minutes and stirred overnight at 80° C. The mixture was filtered and purified by HPLC to give product 613 (3 mg, 9.7%). ¹H NMR (400 MHz, CD₃OD): 8.89 (dd, J=2.2, 1.0 Hz, 1H), 8.77 (d, J=2.3 Hz, 1H), 8.42-8.30 (m, 2H), 7.77 (ddd, J=9.0, 4.5, 2.7 Hz, 1H), 7.36 (dd, J=10.7, 9.0 Hz, 1H), 2.90 (d, J=7.6 Hz, 2H), 2.80-2.63 (m, 1H), 2.56 (s, 3H), 2.48 (s, 3H), 2.21-2.08 (m, 2H), 2.01-1.77 (m, 4H).

### Syntheses of Compounds 614 and 615

Compounds 614 and 615 were synthesized according to the protocol described above using ((1,3-dioxolan-2-yl)methyl) zinc (II) bromide and bicyclo[3.1.1]heptan-6-yl zinc (II) bromide respectively.

### Example 154

### Synthesis of N-(4-fluoro-3-(6-(morpholinomethyl)imidazo[1,2-a]pyrimidin-2-yl)phenyl)-2,4-dimethyloxazole-5-carboxamide (616)

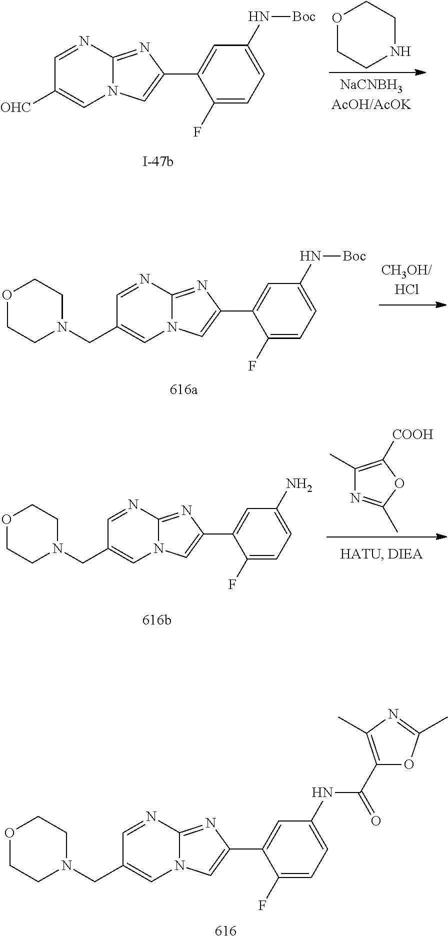

### Synthesis of 616a

To a solution of intermediate I-47b (0.4 g, 1.12 mmol) in methanol(10 mL) was added acetic acid (134.8 mg, 2.24 mmol), potassium acetate (92 mg, 1.12 mmol) and morpholine (489 mg, 5.61 mmol). The mixture was stirred at room temperature for 30 minutes. To this mixture was added sodium cyanoborohydride (141 mg, 2.24 mmol) and the reaction was stirred at room temperature for 15 hours. The mixture was concentrated and diluted with THF: EA=1:1 (100 mL). The organic layer was washed with aqueous sodium bicarbonate (20 mL), brine (20 mL×2), dried over sodium sulfate and concentrated in vacuo. The residue was purified by silica gel chromatography (PE: EA=5:1-THE) to give compound 616a (300 mg, yield, 63%). ¹H NMR (400 MHz, DMSO-d₆) δ 9.52 (s, 1H), 8.92 (d, J=2.01 Hz, 1H), 8.39-8.59 (m, 2H), 8.25 (d, J=4.27 Hz, 1H), 7.35-7.49 (m, 1H), 7.14-7.30 (m, 1H), 3.52-3.62 (m, 6H), 2.44 (s, 4H), 1.50 (s, 9H)

### Synthesis of 616b

A solution of compound 616a (150 mg, 0.35 mmol) in MeOH/HCl (10 mL) was stirred at room temperature for 15 hours. The solution was concentrated and used directly in the next step.

### Synthesis of 616

To a solution of compound 616b (60 mg, 0.42 mmol) in DMF (5 mL) was added HATU (160 mg, 0.42 mmol) and DIEA (180 mg, 1.4 mmol), the reaction mixture was stirred at room temperature for 15 min. Then 2,4-dimethyloxazole-5-carboxylic acid (115 mg, 0.35 mmol) was added and the mixture was stirred at room temperature overnight. The mixture was diluted with Ethyl Acetate (20 mL) and washed with a solution of sodium bicarbonate (20 mL). The organic layer was washed with brine (20 mL×2), dried over sodium sulfate and concentrated. The residue was purified by HPLC to afford compound 616 as a white solid (105 mg, yield, 30%). ¹H NMR (400 MHz, DMSO-d₆) δ 10.34 (s, 1H), 8.93 (d, J=2.01 Hz, 1H), 8.72 (m, 1H), 8.55 (d, J=2.26 Hz, 1H), 8.29 (d, J=4.02 Hz, 1H), 7.76-7.83 (m, 1H), 7.33 (m, 1H), 3.53-3.65 (m, 6H), 2.50-2.50 (m, 1H), 2.45 (s, 4H), 2.41 (s, 3H). MS m/z=451.1 (M+1).

### Syntheses of Compounds 617

Compound 617 was synthesized according to the protocol described above using dimethylamine hydrochloride instead of morpholine.

### Example 115

### Synthesis of N-(4-fluoro-3-(6-(1-morpholinoethyl)imidazo[1,2-a]pyrimidin-2-yl)phenyl)-2,4-dimethyloxazole-5-carboxamide (618)

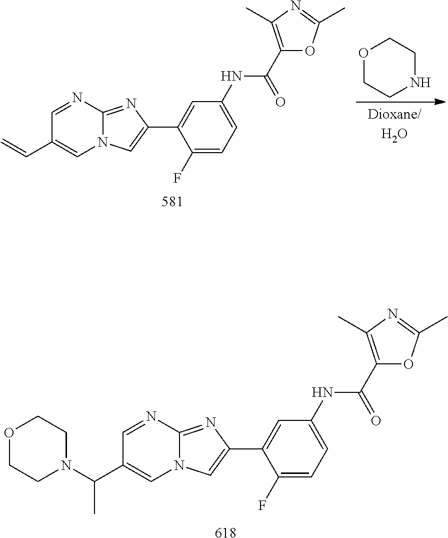

To the solution of compound 581 (200 mg, 0.53 mmol) and morpholine (184.4 mg, 2.12 mmol) in 1,4-dioxane (4 mL) and H₂O (4 mL) was added phenyltrimethylammonium hydroxide (8.3 mg, 0.053 mmol) at room temperature. The mixture was heated to 100° C. and stirred for 4 hours. LCMS shows the starting material was consumed, the mixture was cooled to room temperature and extracted with EtOAc (20 mL) and THF (20 mL). The combined organic layers were dried over Na₂SO₄, concentrated and purified by HPLC to give compound 618 (23 mg, yield: 9.5%) as a pink solid. ¹H NMR (400 MHz, CD3OD) δ 8.80 (d, J=1.76 Hz, 1H), 8.65 (d, J=2.26 Hz, 1H), 8.24 (dd, J=6.53, 2.51 Hz, 1H), 8.18 (d, J=3.76 Hz, 1H), 7.76-7.85 (m, 1H), 7.21 (dd, J=10.67, 9.16 Hz, 1H), 3.67-3.75 (m, 4H), 3.63 (q, J=6.86 Hz, 1H), 2.57 (brs, 2H), 2.54 (s, 3H), 2.47-2.52 (m, 2H), 2.45 (s, 3H). MS m/z=465.2 (M+1).

### Syntheses of Compounds 619

Compound 619 was synthesized according to the protocol described above using dimethylamine hydrochloride instead of morpholine.

TABLE II
Listing of the Exemplified Compounds of the Invention

| | | ¹HNMR and/or mass and/or |
|---|---|---|
| No | Structure | retention time (minutes) |
| 1 | 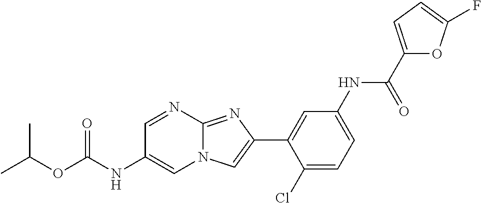 [SMILES: CC(C)OC(=O)Nc1cnc2nc(-c3cc(NC(=O)c4ccc(F)o4)ccc3Cl)cn2c1] | ¹H NMR (400 MHz, DMSO-d₆) δ 10.41 (s, 1H), 10.01 (s, 1H), 9.31 (s, 1H), 8.69 (s, 1H), 8.61 (d, J = 2.7 Hz, 1H), 8.51 (d, J = 2.7 Hz, 1H), 7.87 (dd, J = 2.7, 8.8 Hz, 1H), 7.53 (d, J = 8.7 Hz, 1H), 7.47 (t, J = 3.6 Hz, 1H), 6.11 (dd, J = 3.7, 7.1 Hz, 1H), 5.03-4.85 (m, 1H), 1.29 (d, J = 6.2 Hz, 6H). LCMS m/z = 458.1 (M + 1); RT = 1.64 min, Method 3. |
| 2 | 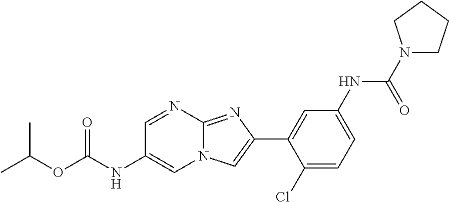 [SMILES: CC(C)OC(=O)Nc1cnc2nc(-c3cc(NC(=O)N4CCCC4)ccc3Cl)cn2c1] | ¹H NMR (400 MHz, DMSO-d₆) δ 9.99 (s, 1H), 9.29 (s, 1H), 8.63 (s, 1H), 8.50 (d, J = 2.7 Hz, 1H), 8.44 (s, 1H), 8.39 (d, J = 2.7 Hz, 1H), 7.70 (dd, J = 2.7, 8.8 Hz, 1H), 7.38 (d, J = 8.8 Hz, 1H), 4.99-4.88 (m, 1H), 3.39 (t, J = 6.6, 4H), 1.85 (t, J = 6.5 Hz, 4H), 1.29 (d, J = 6.2 Hz, 6H). LCMS m/z = 443.1 (M + 1); RT = 1.51, Method 3. |
| 3 | 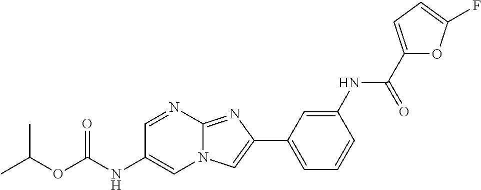 [SMILES: CC(C)OC(=O)Nc1cnc2nc(-c3cccc(NC(=O)c4ccc(F)o4)c3)cn2c1] | ¹H NMR (400 MHz, DMSO-d₆) δ 10.28 (s, 1H), 9.99 (s, 1H). 9.23 (s, 1H), 8.45 (s, 1H), 8.38 (s, 1H), 8.34 (s, 1H), 7.75 (d, J = 7.9 Hz, 1H), 7.66 (d, J = 7.6 Hz, 1H), 7.43 (dd, J = 5.8, 14.9 Hz, 2H), 6.11 (dd, J = 3.4, 6.7 Hz, 1H), 4.94 (dt, J = 6.2, 12.1 Hz, 1H), 1.29 (d, J = 6.2 Hz, 6H). LCMS m/z = 424.1 (M + 1); RT = 1.49, Method 3. |
| 4 | 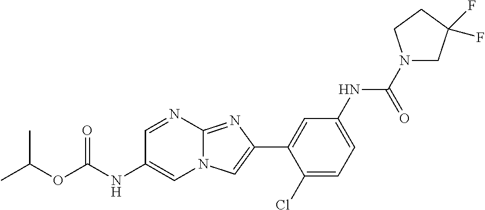 [SMILES: CC(C)OC(=O)Nc1cnc2nc(-c3cc(NC(=O)N4CCC(F)(F)C4)ccc3Cl)cn2c1] | ¹H NMR (400 MHz, DMSO-d₆) δ 10.00 (s, 1H), 9.29 (s, 1H), 8.67 (d, J = 19.1 Hz, 2H), 8.50 (d, J = 2.3 Hz, 1H), 8.39 (d, J = 2.1 Hz, 1H), 7.69 (d, J = 8.8, 1H), 7.41 (d, J = 8.8 Hz, 1H), 4.94 (dt, J = 5.9, 11.8 Hz, 1H), 3.85 (t, J = 13.2 Hz, 3H), 3.65 (t, J = 7.2 Hz, 3H), 1.29 (d, J = 6.2 Hz, 6H). LCMS m/z = 479.1 (M + 1); RT = 1.59, Method 3. |
| 5 | 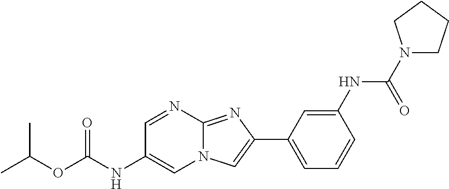 [SMILES: CC(C)OC(=O)Nc1cnc2nc(-c3cccc(NC(=O)N4CCCC4)c3)cn2c1] | ¹H NMR (400 MHz, DMSO-d6) δ 9.97 (s, 1H), 9.21 (s, 1H), 8.43 (d, J = 2.6 Hz, 1H), 8.32 (s, 1H), 8.25 (s, 1H), 8.13 (s, 1H), 7.55 (d, J = 8.1 Hz, 1H), 7.48 (d, J = 7.8 Hz, 1H), 7.29 (t, J = 7.9, 1H), 5.03-4.79 (m, 1H), 3.39 (t, J = 6.6 Hz, 4H), 1.86 (t, J = 6.6 Hz, 4H), 1.29 (d, J = 6.2 Hz, 6H). LCMS m/z = 409.2 (M + 1); RT = 1.38. Method 3. |
| 6 | 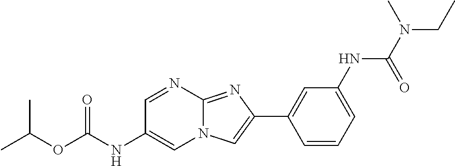 [SMILES: CCN(C)C(=O)Nc1cccc(-c2cn3cc(NC(=O)OC(C)C)cnc3n2)c1] | LCMS m/z = 397.2 (M + 1); RT = 0.76, Method 3. |
| 7 | 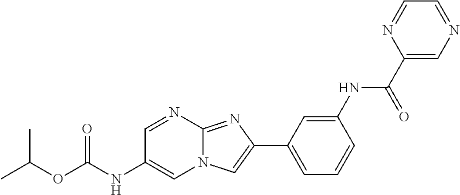 [SMILES: CC(C)OC(=O)Nc1cnc2nc(-c3cccc(NC(=O)c4cnccn4)c3)cn2c1] | LCMS m/z = 418.1 (M + 1); RT = 0.79, Method 3. |
| 8 | 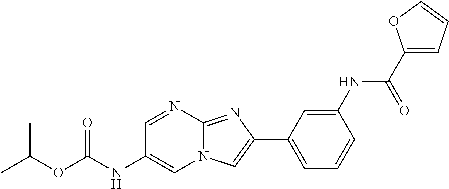 [SMILES: CC(C)OC(=O)Nc1cnc2nc(-c3cccc(NC(=O)c4ccco4)c3)cn2c1] | LCMS m/z = 406.1 (M + 1); RT = 1.44, Method 3. |
| 9 | 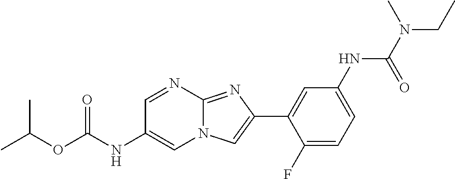 [SMILES: CCN(C)C(=O)Nc1ccc(F)c(-c2cn3cc(NC(=O)OC(C)C)cnc3n2)c1] | LCMS m/z = 415.2 (M + 1); RT = 0.85, Method 3. |
| 10 | 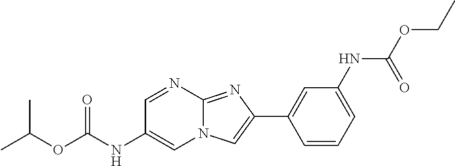 [SMILES: CCOC(=O)Nc1cccc(-c2cn3cc(NC(=O)OC(C)C)cnc3n2)c1] | LCMS m/z = 384.2 (M + 1); RT = 0.84, Method 3. |
| 11 | 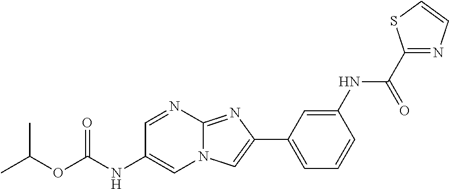 [SMILES: CC(C)OC(=O)Nc1cnc2nc(-c3cccc(NC(=O)c4nccs4)c3)cn2c1] | LCMS m/z = 423.1 (M + 1); RT = 0.85, Method 3. |
| 12 | 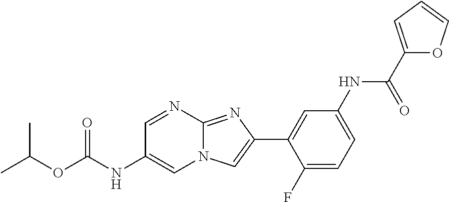 [SMILES: CC(C)OC(=O)Nc1cnc2nc(-c3cc(NC(=O)c4ccco4)ccc3F)cn2c1] | ¹H NMR (400 MHz, DMSO-d₆) δ 10.43 (s, 1H), 10.06 (s, 1H), 9.36 (s, 1H), 8.69 (dd, J = 2.8, 6.9 Hz, 1H), 8.56 (d, J = 2.1 Hz, 1H), 8.45 (d, J = 4.2 Hz, 1H), 8.02 (d, J = 1.0 Hz, 1H), 7.95-7.85 (m, 1H), 7.46 (d, J = 3.4 Hz, 1H), 7.37 (dd, J = 9.0, 10.9 Hz, 1H), 6.78 (dd, J = 1.7, 3.5 Hz, 1H), 5.00 (dt, J = 6.3, 12.5 Hz, 1H), 1.35 (d, J = 6.2 Hz, 6H). LCMS m/z = 424.2 (M + 1); RT = 3.06, Method 3. |
| 13 | 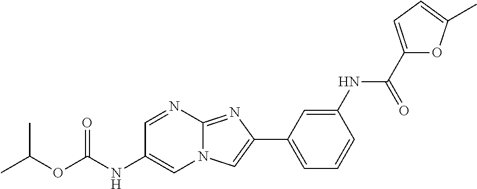 [SMILES: Cc1ccc(C(=O)Nc2cccc(-c3cn4cc(NC(=O)OC(C)C)cnc4n3)c2)o1] | LCMS m/z = 420.2 (M + 1); RT = 0.86, Method 3. |
| 14 | 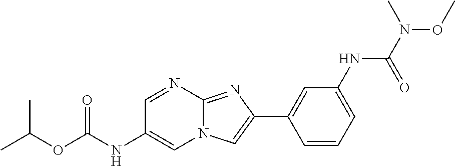 [SMILES: CON(C)C(=O)Nc1cccc(-c2cn3cc(NC(=O)OC(C)C)cnc3n2)c1] | LCMS m/z = 399.2 (M + 1); RT = 0.77, Method 3. |
| 15 | 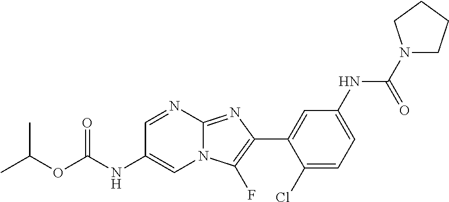 [SMILES: CC(C)OC(=O)Nc1cnc2nc(-c3cc(NC(=O)N4CCCC4)ccc3Cl)c(F)n2c1] | LCMS m/z = 461.1 (M + 1); RT = 1.7, Method 3. |
| 16 | 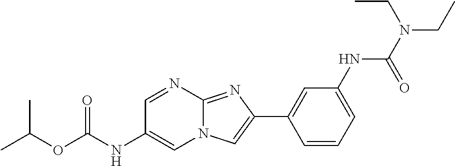 [SMILES: CCN(CC)C(=O)Nc1cccc(-c2cn3cc(NC(=O)OC(C)C)cnc3n2)c1] | LCMS m/z = 411.2 (M + 1); RT = 0.82, Method 3. |
| 17 | 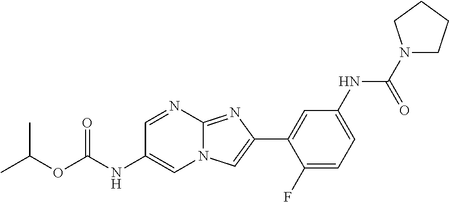 [SMILES: CC(C)OC(=O)Nc1cnc2nc(-c3cc(NC(=O)N4CCCC4)ccc3F)cn2c1] | LCMS m/z = 427.2 (M + 1); RT = 0.86, Method 3. |
| 18 | 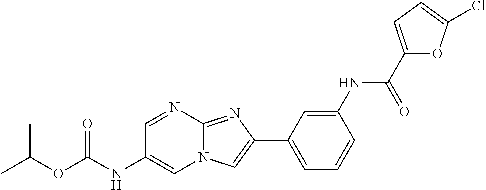 [SMILES: CC(C)OC(=O)Nc1cnc2nc(-c3cccc(NC(=O)c4ccc(Cl)o4)c3)cn2c1] | LCMS m/z = 440.1 (M + 1); RT = 0.91, Method 3. |
| 19 | 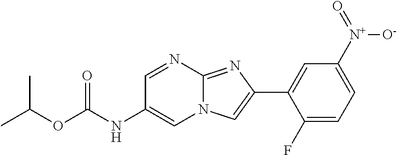 [SMILES: CC(C)OC(=O)Nc1cnc2nc(-c3cc([N+](=O)[O-])ccc3F)cn2c1] | LCMS m/z = 360.1 (M + 1); RT = 1.67, Method 3. |
| 20 | 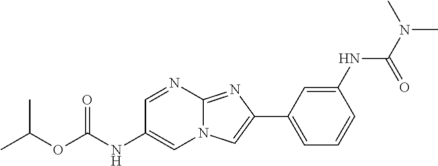 [SMILES: CC(C)OC(=O)Nc1cnc2nc(-c3cccc(NC(=O)N(C)C)c3)cn2c1] | LCMS m/z = 383.2 (M + 1); RT = 0.71, Method 3. |
| 21 | 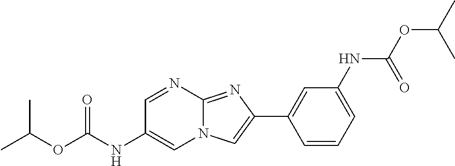 [SMILES: CC(C)OC(=O)Nc1cccc(-c2cn3cc(NC(=O)OC(C)C)cnc3n2)c1] | LCMS m/z = 398.2 (M + 1); RT = 0.9, Method 3. |
| 22 | 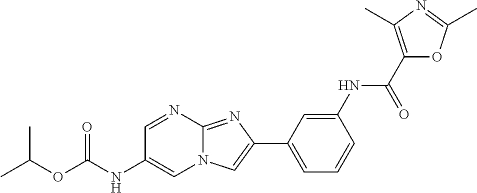 [SMILES: Cc1nc(C)c(C(=O)Nc2cccc(-c3cn4cc(NC(=O)OC(C)C)cnc4n3)c2)o1] | LCMS m/z = 435.2 (M + 1); RT = 0.8, Method 3. |
| 23 | 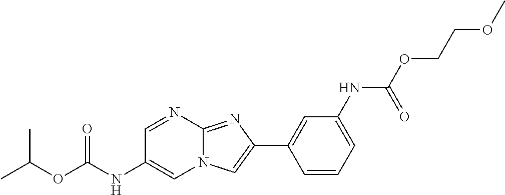 [SMILES: COCCOC(=O)Nc1cccc(-c2cn3cc(NC(=O)OC(C)C)cnc3n2)c1] | LCMS m/z = 414.2 (M + 1); RT = 1.40, Method 3. |
| 24 | 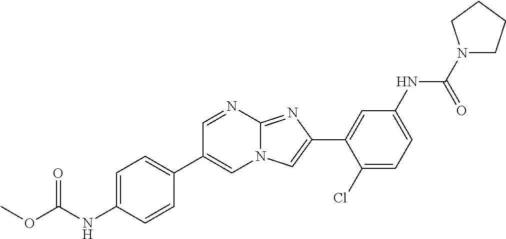 [SMILES: COC(=O)Nc1ccc(-c2cnc3nc(-c4cc(NC(=O)N5CCCC5)ccc4Cl)cn3c2)cc1] | ¹H NMR (600 MHz, DMSO-d₆) δ 9.86 (s, 1H), 9.30 (d, J = 2.6 Hz, 1H), 8.94 (d, J = 2.6 Hz, 1H), 8.55 (s, 1H), 8.47 (d, J = 2.8 Hz, 2H), 7.73 (m, 3H), 7.62 (d, J = 8.5 Hz, 2H), 7.41 (d, J = 8.8 Hz, 1H), 3.70 (s, 3H), 3.40 (t, J = 6.5 Hz, 4H), 1.86 (t, J = 6.4 Hz, 4H). LCMS m/z = 491.2 (M + 1); RT = 1.58, Method 3. |
| 25 | 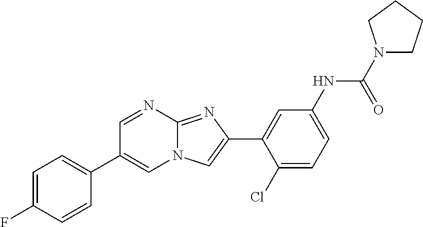 [SMILES: O=C(Nc1ccc(Cl)c(-c2cn3cc(-c4ccc(F)cc4)cnc3n2)c1)N1CCCC1] | ¹H NMR (400 MHz, DMSO-d₆) δ 9.35 (s, 1H), 8.95 (s, 1H), 8.57 (s, 1H), 8.48 (s, 2H), 7.90-7.78 (m, 2H), 7.74 (d, J = 8.8 Hz, 1H), 7.41 (t, J = 8.6 Hz, 3H), 3.40 (m, 4H), 1.86 (m, 4H) LCMS m/z = 436.1 (M + 1); RT = 1.66, Method 3. |
| 26 | 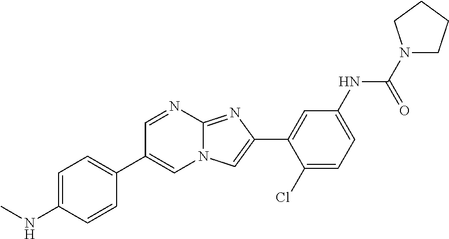 [SMILES: CNc1ccc(-c2cnc3nc(-c4cc(NC(=O)N5CCCC5)ccc4Cl)cn3c2)cc1] | ¹H NMR (600 MHz, DMSO-d₆) δ 9.17 (d, J = 2.6 Hz, 1H), 8.88 (d, J = 2.6, 1H), 8.52 (s, 1H), 8.46 (m, 2H), 7.73 (m, 1H), 7.52 (d, J = 8.6 Hz, 2H), 7.40 (d, J = 8.8 Hz, 1H), 6.68 (d, J = 8.7 Hz, 2H), 5.97 (s, 1H), 3.40 (s, 4H), 2.73 (d, J = 5.0 Hz, 3H), 1.86 (s, 4H). LCMS m/z = 447.2 (M + 1); RT = 1.49, Method 3. |
| 27 | 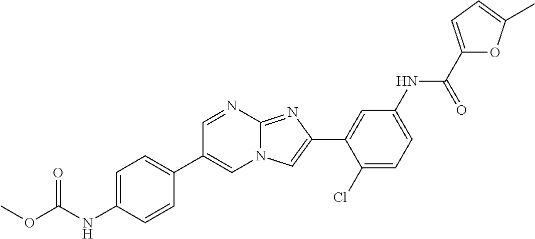 | ¹H NMR (600 MHz, DMSO-d₆) δ 10.31 (s, 1H), 9.86 (s, 1H), 9.32 (d, J = 2.5 Hz, 1H), 8.97 (d, J = 2.5 Hz, 1H), 8.69 (d, J = 2.6 Hz, 1H), 8.60 (s, 1H), 7.93 (dd, J = 2.7, 8.7 Hz, 1H), 7.73 (d, J = 8.6 Hz, 2H), 7.63 (d, J = 8.5 Hz, 2H), 7.55 (d, J = 8.7 Hz, 1H), 7.33 (d, J = 3.3 Hz, 1H), 6.35 (d, J = 2.5 Hz, 1H), 3.70 (s, 3H), 2.40 (s, 3H). LCMS m/z = 502.1 (M + 1); RT = 1.70, Method 3. |
| 28 | 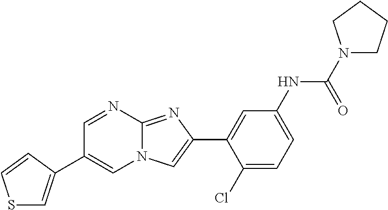 [SMILES: O=C(Nc1ccc(Cl)c(-c2cn3cc(-c4ccsc4)cnc3n2)c1)N1CCCC1] | ¹H NMR (400 MHz, DMSO-d₆) δ 9.41 (d, J = 2.5 Hz, 1H), 9.05 (d, J = 2.5 Hz, 1H), 8.52 (s, 1H), 8.47 (d, J = 2.6 Hz, 2H), 8.08 (dd, J = 1.3, 2.9 Hz, 1H), 7.78 (dd, J = 2.9, 5.0 Hz, 1H), 7.73 (dd, J = 2.7, 8.8 Hz, 1H), 7.63 (dd, J = 1.3, 5.0 Hz, 1H), 7.41 (d, J = 8.8 Hz, 1H), 3.40 (t, J = 6.6 Hz, 4H), 1.86 (t, J = 6.5 Hz, 4H). LCMS m/z = 424.1 (M + 1); RT = 1.62, Method 3. |
| 29 | 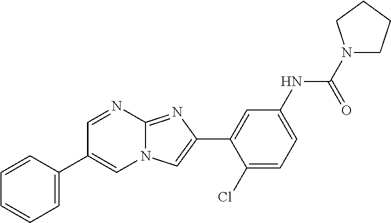 [SMILES: O=C(Nc1ccc(Cl)c(-c2cn3cc(-c4ccccc4)cnc3n2)c1)N1CCCC1] | ¹H NMR (400 MHz, DMSO-d₆) δ 9.37 (s, 1H), 8.97 (s, 1H), 8.58 (s, 1H), 8.48 (d, J = 4.4 Hz, 2H), 7.77 (dd, J = 7.8, 20.3 Hz, 3H), 7.55 (d, J = 5.0 Hz, 2H), 7.44 (dd, J = 8.2, 25.3 Hz, 2H), 3.40 (m, 4H), 1.86 (m, 4H). LCMS m/z = 418.1 (M + 1); RT = 1.65. Method 3. |
| 30 | 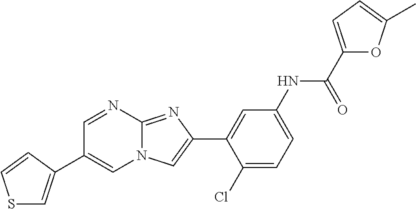 [SMILES: Cc1ccc(C(=O)Nc2ccc(Cl)c(-c3cn4cc(-c5ccsc5)cnc4n3)c2)o1] | ¹H NMR (400 MHz, DMSO-d₆) δ 10.31 (s, 1H), 9.42 (d, J = 1.9 Hz, 1H), 9.08 (d, J = 2.0 Hz, 1H), 8.70 (d, J = 2.4 Hz, 1H), 8.57 (s, 1H), 8.10 (s, 1H), 7.93 (m, 1H), 7.78 (m, 1H), 7.64 (d, J = 5.0 Hz, 1H), 7.55 (d, J = 8.7 Hz, 1H), 7.33 (d, J = 3.2 Hz, 1H), 6.35 (d, J = 3.2 Hz, 1H), 2.40 (s, 3H). LCMS m/z = 435.1 (M + 1); RT = 1.78, Method 3. |
| 31 | 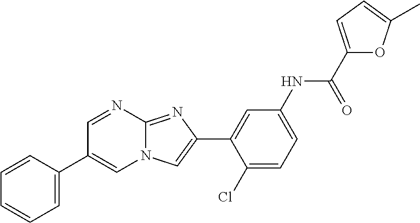 [SMILES: Cc1ccc(C(=O)Nc2ccc(Cl)c(-c3cn4cc(-c5ccccc5)cnc4n3)c2)o1] | ¹H NMR (400 MHz, DMSO-d₆) δ 10.33 (s, 1H), 9.38 (d, J = 2.1 Hz, 1H), 8.99 (d, J = 2.0 Hz, 1H), 8.71 (d, J = 2.5 Hz, 1H), 8.62 (s, 1H), 7.95 (dd, J = 2.4, 8.7 Hz, 1H), 7.80 (d, J = 7.8 Hz, 2H), 7.56 (t, J = 8.3 Hz, 3H), 7.47 (t, J = 7.3 Hz, 1H), 7.34 (d, J = 3.3 Hz, 1H), 6.35 (d, J = 3.3 Hz, 1H), 2.40 (s, 3H) LCMS m/z = 429.1 (M + 1); RT = 1.82, Method 3. |
| 32 | 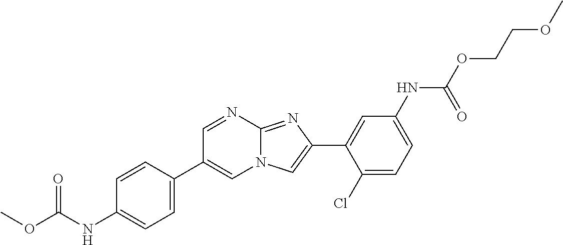 | ¹H NMR (600 MHz, DMSO-d₆) δ 10.01 (s, 1H), 9.86 (s, 1H), 9.30 (s, 1H), 8.95 (s, 1H), 8.55 (s, 1H), 8.47 (s, 1H), 7.72 (d, J = 6.0 Hz, 2H), 7.62 (d, J = 6.6 Hz, 2H), 7.50 (dt, J = 5.7, 8.5 Hz, 2H), 4.24 (s, 2H), 3.70 (m, 3H), 3.59 (m, 3H). 3.30 (s, 3H). LCMS m/z = 496.1 (M + 1); RT = 1.6, Method 3. |
| 33 | 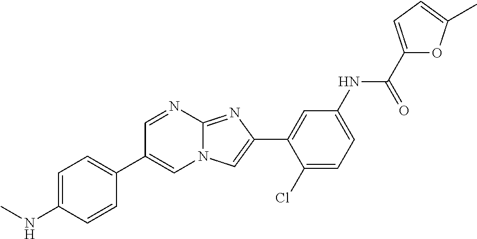 | LCMS m/z = 458.1 (M + 1); RT = 3.18, Method 3. |
| 34 | 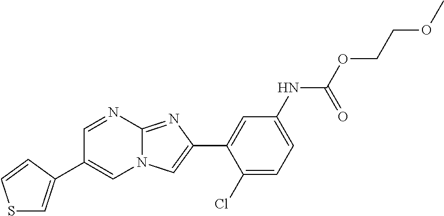 [SMILES: COCCOC(=O)Nc1ccc(Cl)c(-c2cn3cc(-c4ccsc4)cnc3n2)c1] | ¹H NMR (400 MHz, DMSO-d₆) δ 9.15 (d, J = 2.4 Hz, 1H), 8.95 (d, J = 2.4 Hz, 1H), 8.41 (s, 1H), 7.99 (d, J = 2.7 Hz, 1H), 7.86 (d, J = 1.6 Hz, 1H), 7.61 (m, 2H), 7.54 (d, J = 4.0 Hz, 1H), 7.41 (d, J = 8.8 Hz, 1H), 4.27 (m, 2H), 3.64 (m, 2H), 3.38 (s, 3H). LCMS m/z = 429.1 (M + 1); RT = 1.7, Method 3. |
| 35 | 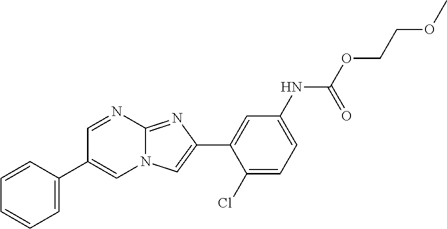 [SMILES: COCCOC(=O)Nc1ccc(Cl)c(-c2cn3cc(-c4ccccc4)cnc3n2)c1] | ¹H NMR (400 MHz, DMSO-d₆) δ 10.03 (s, 1H), 9.37 (d, J = 2.5 Hz, 1H), 8.98 (d, J = 2.5 Hz, 1H), 8.58 (s, 1H), 8.48 (s, 1H), 7.80 (d, J = 7.9 Hz, 2H), 7.52 (m, 5H), 4.24 (m, 2H), 3.59 (m, 2H), 3.30 (s, 3H). LCMS m/z = 423.1 (M + 1); RT = 1.73, Method 3. |
| 36 | 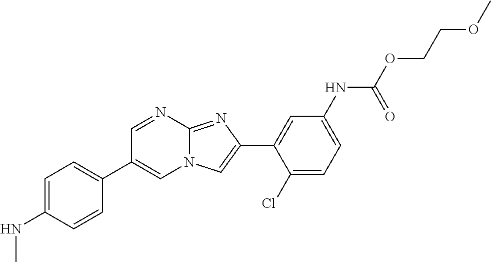 [SMILES: CNc1ccc(-c2cnc3nc(-c4cc(NC(=O)OCCOC)ccc4Cl)cn3c2)cc1] | ¹H NMR (600 MHz, DMSO-d₆) δ 10.00 (s, 1H), 9.17 (d, J = 2.6 Hz, 1H), 8.89 (d, J = 2.6 Hz, 1H), 8.51 (s, 1H), 8.46 (d, J = 2.5 Hz, 1H), 7.52 (dd, J = 5.6, 11.8 Hz, 3H), 7.48 (d, J = 8.7 Hz, 1H), 6.68 (d, J = 8.7 Hz, 2H), 5.97 (d, J = 5.1 Hz, 1H), 4.24 (dd, J = 3.8, 5.4 Hz, 2H), 3.63-3.54 (m, 2H). 3.30 (s, 3H), 2.73 (d, J = 5.0 Hz, 3H). LCMS m/z = 452.1 (M + 1); RT = 1.43, Method 3. |
| 37 | 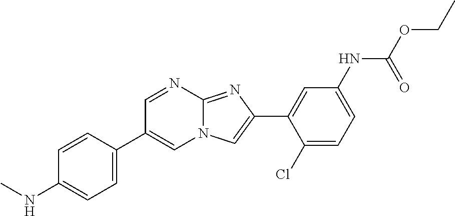 [SMILES: CCOC(=O)Nc1ccc(Cl)c(-c2cn3cc(-c4ccc(NC)cc4)cnc3n2)c1] | ¹H NMR (600 MHz, DMSO-d₆) δ 9.87 (s, 1H), 9.17 (d, J = 2.6 Hz, 1H), 8.89 (d, J = 2.6 Hz, 1H), 8.51 (s, 1H), 8.44 (d, J = 2.5 Hz, 1H), 7.53 (d, J = 8.6 Hz, 3H), 7.47 (d, J = 8.7 Hz, 1H), 6.68 (d, J = 8.6 Hz, 2H), 5.97 (t, J = 5.0 Hz, 1H), 4.16 (q, J = 7.1 Hz, 2H), 2.73 (d, J = 5.0, 3H), 1.27 (t, J = 7.1 Hz, 3H). LCMS m/z = 422.1 (M + 1); RT = 1.49, Method 3. |
| 38 | 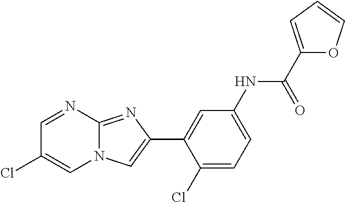 [SMILES: O=C(Nc1ccc(Cl)c(-c2cn3cc(Cl)cnc3n2)c1)c1ccco1] | LCMS m/z = 372.9 (M + 1); RT = 1.59, Method 3. |
| 39 | 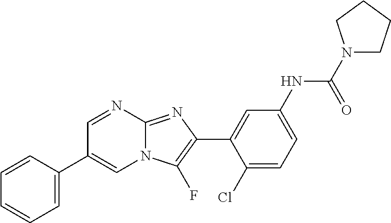 [SMILES: O=C(Nc1ccc(Cl)c(-c2nc3ncc(-c4ccccc4)cn3c2F)c1)N1CCCC1] | ¹H NMR (600 MHz, DMSO-d₆) δ 9.14 (d, J = 2.2 Hz, 1H), 8.98 (d, J = 2.5 Hz, 1H), 8.45 (s, 1H), 8.00 (d, J = 2.7 Hz, 1H), 7.89-7.84 (m, 2H), 7.74 (dd, J = 2.7, 8.8 Hz, 1H), 7.58-7.52 (m, 2H), 7.50-7.43 (m, 2H), 3.38 (t, J = 6.7 Hz, 4H), 1.85 (m, 4H). LCMS m/z = 436.1 (M + 1); RT = 1.82, Method 3. |
| 40 | 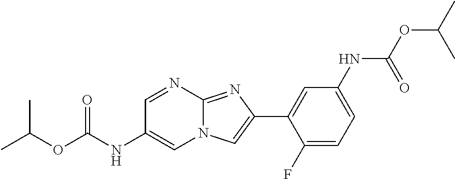 [SMILES: CC(C)OC(=O)Nc1ccc(F)c(-c2cn3cc(NC(=O)OC(C)C)cnc3n2)c1] | LCMS m/z = 416.2 (M + 1); RT = 3.33, Method 3. |
| 41 | 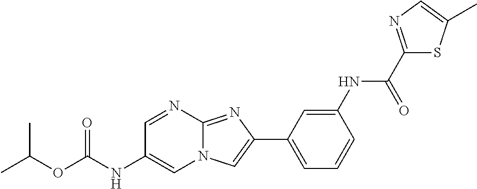 [SMILES: Cc1cnc(C(=O)Nc2cccc(-c3cn4cc(NC(=O)OC(C)C)cnc4n3)c2)s1] | LCMS m/z = 437.2 (M + 1); RT = 3.07, Method 3. |
| 42 | 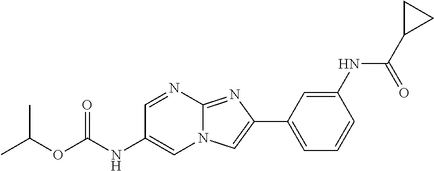 [SMILES: CC(C)OC(=O)Nc1cnc2nc(-c3cccc(NC(=O)C4CC4)c3)cn2c1] | LCMS m/z = 380.0 (M + 1); RT = 0.83, Method 3. |
| 43 | 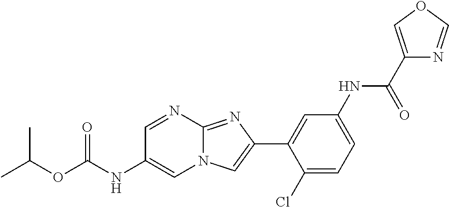 [SMILES: CC(C)OC(=O)Nc1cnc2nc(-c3cc(NC(=O)c4cocn4)ccc3Cl)cn2c1] | LCMS m/z = 441.2 (M + 1); RT = 3.04, Method 1. |
| 44 | 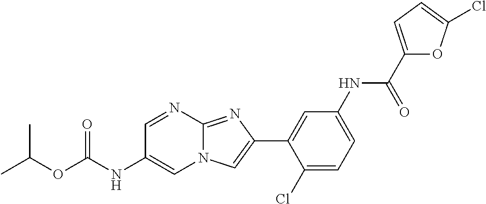 [SMILES: CC(C)OC(=O)Nc1cnc2nc(-c3cc(NC(=O)c4ccc(Cl)o4)ccc3Cl)cn2c1] | ¹H NMR (400 MHz, CD₃OD) δ 9.45 (s, 1H), 8.66 (d, J = 2.6 Hz, 1H), 8.44 (s, 1H), 8.20 (d, J = 2.6 Hz, 1H), 7.82 (dd, J = 8.8, 2.6 Hz, 1H), 7.54 (d, J = 8.8 Hz, 1H), 7.32 (d, J = 3.7 Hz, 1H), 6.51 (d, J = 3.6 Hz, 1H), 5.04 (hept, J = 6.2 Hz, 1H), 1.34 (d, J = 6.2 Hz, 6H). LCMS m/z = 474.1 (M + 1); RT = 3.32, Method 1. |
| 45 | 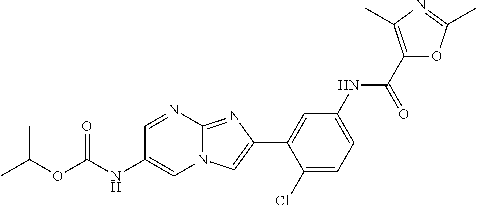 [SMILES: Cc1nc(C)c(C(=O)Nc2ccc(Cl)c(-c3cn4cc(NC(=O)OC(C)C)cnc4n3)c2)o1] | ¹H NMR (400 MHz, CD₃OD) δ 9.52 (s, 1H), 8.76-8.68 (m, 1H), 8.50 (s, 1H), 8.28 (d, J = 2.6 Hz, 1H), 7.77 (dd, J = 8.8, 2.6 Hz, 1H), 7.64-7.53 (m, 1H), 5.04 (hept, J = 6.2 Hz, 1H), 2.55 (s, 3H), 2.46 (s, 3H), 1.35 (d, J = 6.2 Hz, 6H). LCMS m/z = 469.2 (M + 1); RT = 3.18, Method 1. |
| 46 | 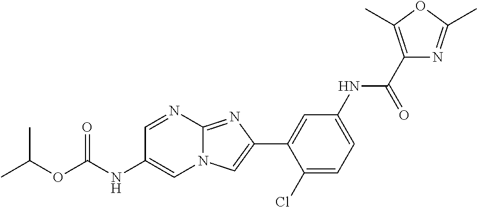 [SMILES: Cc1nc(C(=O)Nc2ccc(Cl)c(-c3cn4cc(NC(=O)OC(C)C)cnc4n3)c2)c(C)o1] | LCMS m/z = 469.2 (M + 1); RT = 3.29, Method 1. |
| 47 | 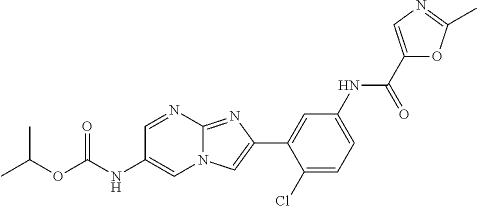 [SMILES: Cc1ncc(C(=O)Nc2ccc(Cl)c(-c3cn4cc(NC(=O)OC(C)C)cnc4n3)c2)o1] | ¹H NMR (400 MHz, CD₃OD) δ 9.57 (s, 1H), 8.76 (dd, J = 2.6, 1.1 Hz, 1H), 8.52 (s, 1H), 8.30 (d, J = 2.4 Hz, 1H), 7.83-7.73 (m, 2H), 7.62 (d, J = 8.8 Hz, 1H), 5.05 (hept, J = 6.1 Hz, 1H), 2.59 (s, 3H), 1.35 (d, J = 6.3 Hz, 6H). LCMS m/z = 454.9 (M + 1); RT = 0.90, Method 2. |
| 48 | 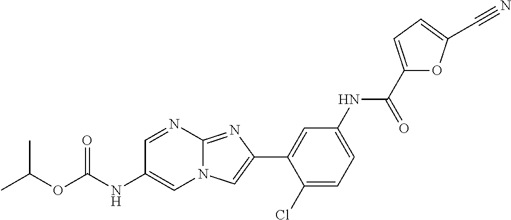 [SMILES: CC(C)OC(=O)Nc1cnc2nc(-c3cc(NC(=O)c4ccc(C#N)o4)ccc3Cl)cn2c1] | ¹H NMR (400 MHz, CD₃OD) δ 9.35 (s, 1H), 8.55 (d, J = 2.7 Hz, 1H), 8.43 (s, 1H), 8.22 (d, J = 2.7 Hz, 1H), 7.88 (dd, J = 8.8, 2.6 Hz, 1H), 7.54 (d, J = 8.8 Hz, 1H), 7.46 (d, J = 3.8 Hz, 1H), 7.40 (d, J = 3.8 Hz, 1H), 5.03 (hept, J = 6.2 Hz, 1H), 1.34 (d, J = 6.3 Hz, 6H). LCMS m/z = 464.9 (M + 1); RT = 1.01, Method 2. |
| 49 | 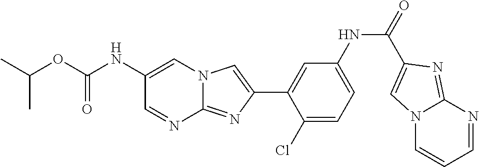 [SMILES: CC(C)OC(=O)Nc1cnc2nc(-c3cc(NC(=O)c4cn5cccnc5n4)ccc3Cl)cn2c1] | LCMS m/z = 491.0 (M + 1); RT = 0.89, Method 2 |
| 50 | 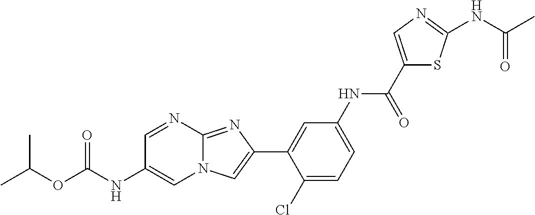 [SMILES: CC(=O)Nc1ncc(C(=O)Nc2ccc(Cl)c(-c3cn4cc(NC(=O)OC(C)C)cnc4n3)c2)s1] | ¹H NMR (400 MHz, CD₃OD) δ 9.60 (s, 1H), 8.80 (d, J = 2.6 Hz, 1H), 8.53 (s, 1H), 8.31 (d, J = 2.6 Hz, 1H), 8.22 (s, 1H), 7.74 (dd, J = 8.8, 2.5 Hz, 1H), 7.63 (d, J = 8.8 Hz, 1H), 5.06 (hept, J = 6.5 Hz, 1H), 2.25 (s, 3H), 1.35 (d, J = 6.2 Hz, 6H). LCMS m/z = 513.9 (M + 1); RT = 0.93, Method 2. |
| 51 | 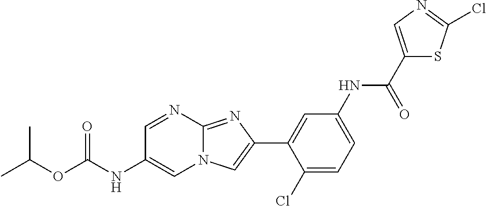 [SMILES: CC(C)OC(=O)Nc1cnc2nc(-c3cc(NC(=O)c4cnc(Cl)s4)ccc3Cl)cn2c1] | ¹H NMR (400 MHz, CD₃OD) δ 9.46 (s, 1H), 8.66 (d, J = 2.5 Hz, 1H), 8.49 (s, 1H), 8.33 (s, 1H), 8.26 (d, J = 2.6 Hz, 1H), 7.80 (dd, J = 8.8, 2.6 Hz, 1H), 7.59 (d, J = 8.8 Hz, 1H), 5.10-4.99 (m, 1H), 1.34 (d, J = 6.1 Hz, 6H). LCMS m/z = 490.9 (M + 1); RT = 1.08, Method 2. |
| 52 | 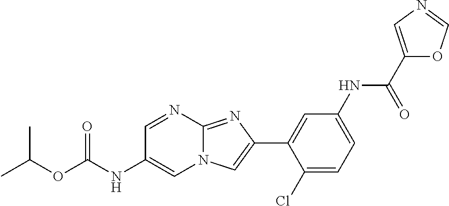 [SMILES: CC(C)OC(=O)Nc1cnc2nc(-c3cc(NC(=O)c4cnco4)ccc3Cl)cn2c1] | ¹H NMR (400 MHz, CD₃OD) δ 9.47 (s, 1H), 8.66 (d, J = 2.6 Hz, 1H), 8.49 (s, 1H), 8.43 (s, 1H), 8.28 (d, J = 2.5 Hz, 1H), 7.92 (s, 1H), 7.82 (dd, J = 8.8, 2.7 Hz, 1H), 7.59 (d, J = 8.7 Hz, 1H), 5.04 (hept, J = 6.1 Hz, 1H), 1.34 (d, J = 6.3 Hz, 6H). LCMS m/z = 440.9 (M + 1); RT = 0.88, Method 2. |
| 53 | 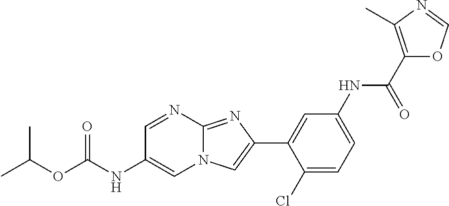 [SMILES: Cc1ncoc1C(=O)Nc1ccc(Cl)c(-c2cn3cc(NC(=O)OC(C)C)cnc3n2)c1] | LCMS m/z = 455.3 (M + 1); RT = 1.57, Method 3. |
| 54 | 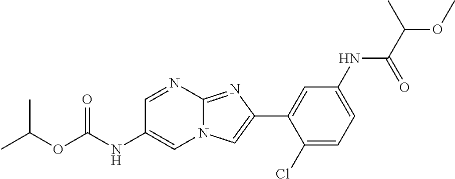 [SMILES: COC(C)C(=O)Nc1ccc(Cl)c(-c2cn3cc(NC(=O)OC(C)C)cnc3n2)c1] | ¹H NMR (400 MHz, CD₃OD) δ 9.56 (s, 1H), 8.75 (d, J = 2.6 Hz, 1H), 8.50 (s, 1H), 8.23 (d, J = 2.6 Hz, 1H), 7.73 (dd, J = 8.8, 2.6 Hz, 1H), 7.59 (d, J = 8.8 Hz, 1H), 5.05 (hept, J = 6.2 Hz, 1H), 3.92 (q, J = 6.7 Hz, 1H), 3.45 (s, 3H), 1.43 (d, J = 6.8 Hz, 3H), 1.35 (d, J = 6.2 Hz, 6H). LCMS m/z = 432 (M + 1); RT = 0.93, Method 2. |
| 55 | 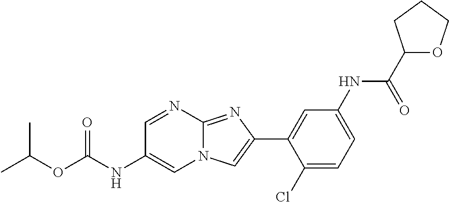 [SMILES: CC(C)OC(=O)Nc1cnc2nc(-c3cc(NC(=O)C4CCCO4)ccc3Cl)cn2c1] | ¹H NMR (400 MHz, CD₃OD) δ 9.55 (s, 1H), 8.75 (d, J = 2.6 Hz, 1H), 8.50 (s, 1H), 8.22 (d, J = 2.7 Hz, 1H), 7.73 (dd, J = 8.8, 2.6 Hz, 1H), 7.59 (d, J = 8.8 Hz, 1H), 5.05 (hept, J = 6.1 Hz, 1H), 4.46 (dd, J = 8.3, 5.9 Hz, 1H), 4.11 (dt, J = 8.0, 6.6 Hz, 1H), 3.95 (dt, J = 8.2, 6.8 Hz, 1H), 2.36 (dq, J = 12.7, 7.5 Hz, 1H), 2.10 (ddt, J = 12.3, 8.1, 6.1 Hz, 1H), 2.02-1.92 (m, 2H), 1.35 (d, J = 6.1 Hz. 6H). LCMS m/z = 444 (M + 1): RT = 0.95, Method 2. |
| 56 | 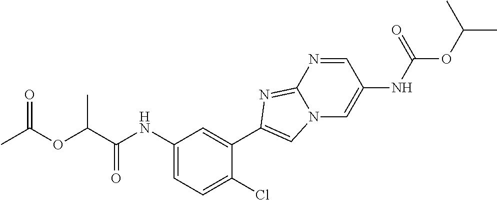 [SMILES: CC(=O)OC(C)C(=O)Nc1ccc(Cl)c(-c2cn3cc(NC(=O)OC(C)C)cnc3n2)c1] | LCMS m/z = 460 (M + 1); RT = 0.93, Method 2. |
| 57 | 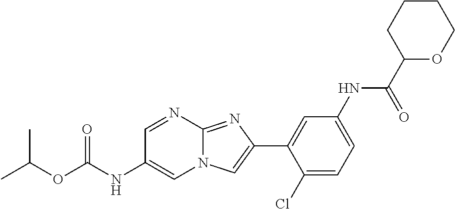 [SMILES: CC(C)OC(=O)Nc1cnc2nc(-c3cc(NC(=O)C4CCCCO4)ccc3Cl)cn2c1] | ¹H NMR (400 MHz, CD₃OD) δ 9.55 (s, 1H), 8.74 (d, J = 2.6 Hz, 1H), 8.49 (s, 1H), 8.20 (d, J = 2.6 Hz, 1H), 7.72 (dd, J = 8.8, 2.6 Hz, 1H), 7.57 (d, J = 8.8 Hz, 1H), 5.05 (hept, J = 6.2 Hz, 1H), 4.15 (ddd, J = 11.6, 3.9, 2.0 Hz, 1H), 3.97 (dd, J = 11.4, 2.5 Hz, 1H), 3.60 (td, J = 11.0, 3.0 Hz, 1H), 2.11-2.03 (m, 1H), 1.99-1.91 (m, 1H), 1.64 (tdd, J = 16.2, 7.9, 3.7 Hz, 3H), 1.56-1.47 (m, 1H), 1.34 (d, J = 6.3 Hz, 6H). LCMS m/z = 458 (M + 1); RT = 1.01, Method 2. |
| 58 | 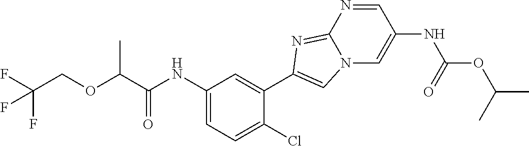 [SMILES: CC(C)OC(=O)Nc1cnc2nc(-c3cc(NC(=O)C(C)OCC(F)(F)F)ccc3Cl)cn2c1] | ¹H NMR (400 MHz, CD₃OD) δ 9.54 (s, 1H), 8.73 (d, J = 2.6 Hz, 1H), 8.50 (s, 1H), 8.22 (d, J = 2.6 Hz, 1H), 7.71 (dd, J = 8.8, 2.5 Hz, 1H), 7.59 (d, J = 8.7 Hz, 1H), 5.05 (hept, J = 6.0 Hz, 1H), 4.26 (q, J = 6.8 Hz, 1H), 4.17 (dq, J = 12.7, 8.9 Hz, 1H), 4.06 (dq, J = 12.7, 8.8 Hz, 1H), 1.51 (d, J = 6.7 Hz, 3H), 1.34 (d, J = 6.2 Hz, 6H). LCMS m/z = 500 (M + 1); RT = 1.07, Method 2. |
| 59 | 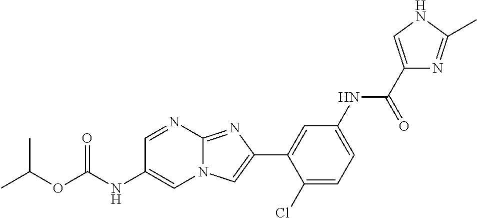 [SMILES: Cc1nc(C(=O)Nc2ccc(Cl)c(-c3cn4cc(NC(=O)OC(C)C)cnc4n3)c2)c[nH]1] | LCMS m/z = 454 (M + 1); RT = 0.74, Method 2. |
| 60 | 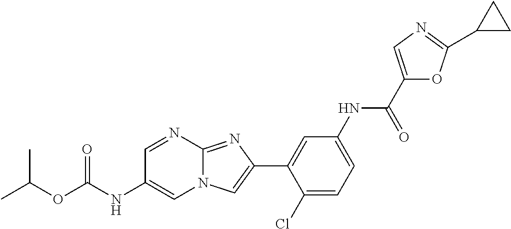 [SMILES: CC(C)OC(=O)Nc1cnc2nc(-c3cc(NC(=O)c4cnc(C5CC5)o4)ccc3Cl)cn2c1] | ¹H NMR (400 MHz, CD₃OD) δ 9.52 (s, 1H), 8.71 (d, J = 2.6 Hz, 1H), 8.50 (s, 1H), 8.29 (d, J = 2.6 Hz, 1H), 7.79 (dd, J = 8.8, 2.6 Hz, 1H), 7.76 (s, 1H), 7.60 (d, J = 8.8 Hz, 1H), 5.05 (hept, J = 6.1 Hz, 1H), 2.23 (p, J = 6.6 Hz, 1H), 1.34 (d, J = 6.3 Hz, 6H), 1.21 (d, J = 6.7 Hz, 4H). LCMS m/z = 481.2 (M + 1); RT = 3.22, Method 1. |
| 61 | 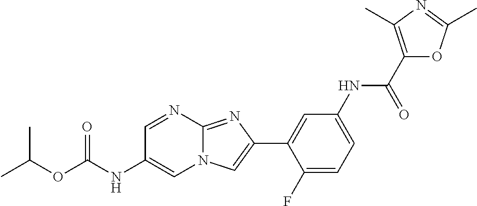 [SMILES: Cc1nc(C)c(C(=O)Nc2ccc(F)c(-c3cn4cc(NC(=O)OC(C)C)cnc4n3)c2)o1] | LCMS m/z = 453.2 (M + 1); RT = 3.11, Method 1. |
| 62 | 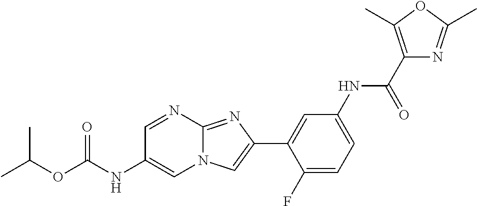 [SMILES: Cc1nc(C(=O)Nc2ccc(F)c(-c3cn4cc(NC(=O)OC(C)C)cnc4n3)c2)c(C)o1] | LCMS m/z = 453.2 (M + 1); RT = 3.23, Method 1. |
| 63 | 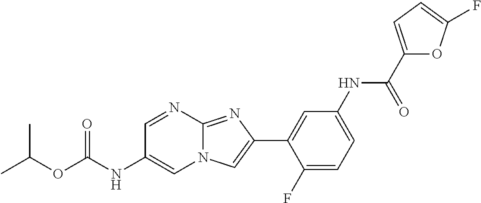 [SMILES: CC(C)OC(=O)Nc1cnc2nc(-c3cc(NC(=O)c4ccc(F)o4)ccc3F)cn2c1] | ¹H NMR (400 MHz, CD₃OD) δ 9.32 (s, 1H), 8.53 (d, J = 2.5 Hz, 1H), 8.27 (d, J = 3.6 Hz, 1H), 8.21 (dd, J = 6.5, 2.7 Hz, 1H), 7.85 (ddd, J = 8.9, 4.3, 2.7 Hz, 1H), 7.31 (t, J = 3.5 Hz, 1H), 7.26 (dd, J = 10.9, 8.9 Hz, 1H), 5.90 (dd, J = 7.0, 3.6 Hz, 1H), 5.08-4.98 (m, 1H), 1.34 (d, J = 6.2 Hz, 6H). LCMS m/z = 442.2 (M + 1); RT = 3.18, Method 1. |
| 64 | 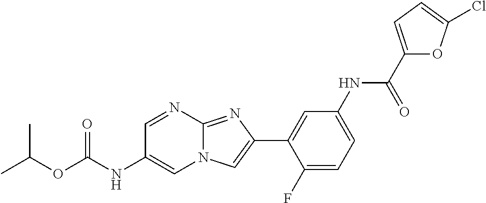 [SMILES: CC(C)OC(=O)Nc1cnc2nc(-c3cc(NC(=O)c4ccc(Cl)o4)ccc3F)cn2c1] | ¹H NMR (400 MHz, CD₃OD) δ 9.38 (s, 1H), 8.59 (d, J = 2.6 Hz, 1H), 8.31 (d, J = 3.3 Hz, 1H), 8.26 (dd, J = 6.7, 2.7 Hz, 1H), 7.83 (ddd, J = 8.8, 4.5, 2.7 Hz, 1H), 7.34 (d, J = 3.6 Hz, 1H), 7.29 (dd, J = 10.8, 9.0 Hz, 1H), 6.57 (d, J = 3.6 Hz, 1H), 5.03 (hept, J = 6.0 Hz, 1H), 1.34 (d, J = 6.3 Hz, 6H). LCMS m/z = 458.1 (M + 1); RT = 3.28, Method 1. |
| 65 | 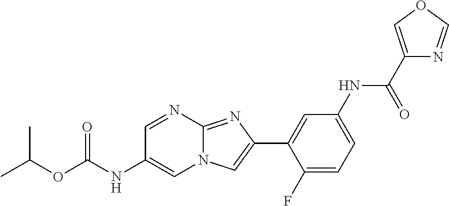 [SMILES: CC(C)OC(=O)Nc1cnc2nc(-c3cc(NC(=O)c4cocn4)ccc3F)cn2c1] | ¹H NMR (400 MHz, CD₃OD) δ 9.34 (s, 1H), 8.57 (s, 1H), 8.54 (d, J = 2.6 Hz, 1H), 8.34-8.30 (m, 2H), 8.28 (d, J = 3.5 Hz, 1H), 7.87 (ddd, J = 8.9, 4.4, 2.7 Hz, 1H), 7.28 (dd, J = 10.8, 8.9 Hz, 1H), 5.03 (hept, J = 6.2 Hz, 1H), 1.34 (d, J = 6.3 Hz, 6H). LCMS m/z = 425.2 (M + 1); RT = 2.98, Method 1. |
| 66 | 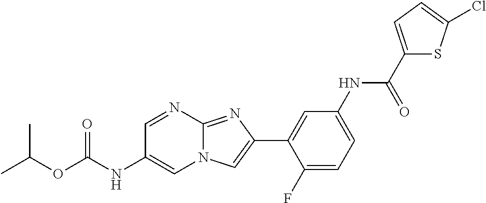 [SMILES: CC(C)OC(=O)Nc1cnc2nc(-c3cc(NC(=O)c4ccc(Cl)s4)ccc3F)cn2c1] | LCMS m/z = 473.9 (M + 1); RT = 1.09, Method 2. |
| 67 | 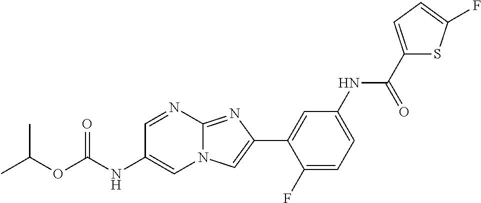 [SMILES: CC(C)OC(=O)Nc1cnc2nc(-c3cc(NC(=O)c4ccc(F)s4)ccc3F)cn2c1] | ¹H NMR (400 MHz, CD₃OD) δ 9.44 (s, 1H), 8.65 (d, J = 2.4 Hz, 1H), 8.34 (d, J = 3.0 Hz, 1H), 8.24 (dd, J = 6.5, 2.7 Hz, 1H), 7.77 (ddd, J = 8.9, 4.4, 2.7 Hz, 1H), 7.69-7.65 (m, 1H), 7.28 (dd, J = 10.7, 9.0 Hz, 1H), 6.67 (dd, J = 4.4, 1.7 Hz, 1H), 5.03 (hept, J = 6.2 Hz, 1H), 1.34 (d, J = 6.2 Hz, 6H). LCMS m/z = 458 (M + 1); RT = 1.04, Method 2. |
| 68 | 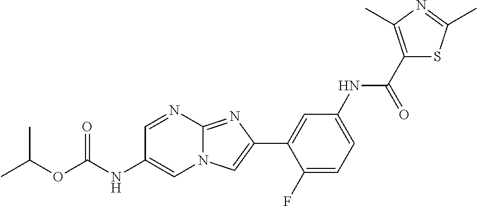 [SMILES: Cc1nc(C)c(C(=O)Nc2ccc(F)c(-c3cn4cc(NC(=O)OC(C)C)cnc4n3)c2)s1] | LCMS m/z = 469.3 (M + 1); RT = 1.61, Method 3. |
| 69 | 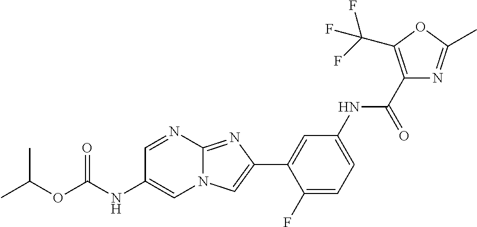 [SMILES: Cc1nc(C(=O)Nc2ccc(F)c(-c3cn4cc(NC(=O)OC(C)C)cnc4n3)c2)c(C(F)(F)F)o1] | LCMS m/z = 507.2 (M + 1); RT = 1.96, Method 3. |
| 70 | 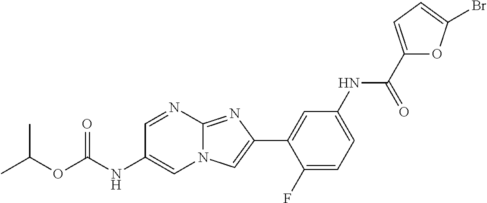 [SMILES: CC(C)OC(=O)Nc1cnc2nc(-c3cc(NC(=O)c4ccc(Br)o4)ccc3F)cn2c1] | LCMS m/z = 502.1 (M + 1); RT = 1.68, Method 3. |
| 71 | 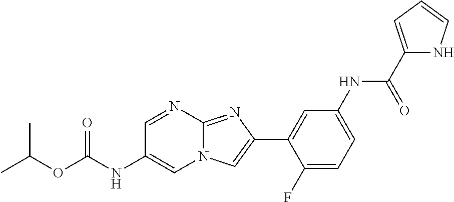 [SMILES: CC(C)OC(=O)Nc1cnc2nc(-c3cc(NC(=O)c4ccc[nH]4)ccc3F)cn2c1] | LCMS m/z = 423.2 (M + 1); RT = 1.72, Method 3. |
| 72 | 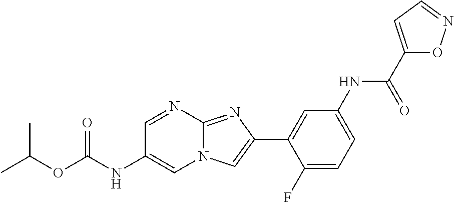 [SMILES: CC(C)OC(=O)Nc1cnc2nc(-c3cc(NC(=O)c4ccno4)ccc3F)cn2c1] | LCMS m/z = 424.9 (M + 1); RT = 0.94, Method 2. |
| 73 | 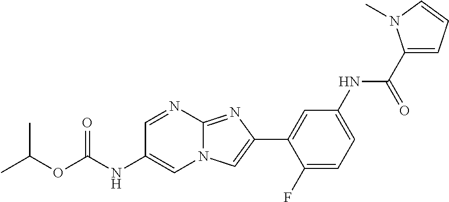 [SMILES: CC(C)OC(=O)Nc1cnc2nc(-c3cc(NC(=O)c4cccn4C)ccc3F)cn2c1] | ¹H NMR (400 MHz, CD₃OD) δ 9.53- 9.42 (m, 1H), 8.69 (d, J = 2.5 Hz, 1H), 8.40 (d, J = 2.8 Hz, 1H), 8.27 (dd, J = 6.7, 2.6 Hz, 1H), 7.71 (ddd, J = 8.9, 4.3, 2.6 Hz, 1H), 7.30 (dd, J = 10.7, 9.0 Hz, 1H), 7.02 (dd, J = 4.0, 1.6 Hz, 1H), 6.93 (t, J = 2.1 Hz, 1H), 6.15 (dd, J = 4.0, 2.6 Hz, 1H), 5.04 (hept, J = 6.2 Hz, 1H), 1.34 (d, J = 6.3 Hz, 6H). LCMS m/z = 437.1 (M + 1); RT = 1.81, Method 3. |
| 74 | 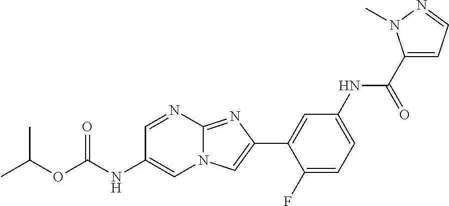 [SMILES: CC(C)OC(=O)Nc1cnc2nc(-c3cc(NC(=O)c4ccnn4C)ccc3F)cn2c1] | ¹H NMR (400 MHz, CD₃OD) δ 9.49 (s, 1H), 8.69 (d, J = 2.5 Hz, 1H), 8.40 (d, J = 2.9 Hz, 1H), 8.34 (dd, J = 6.5, 2.7 Hz, 1H), 7.77 (ddd, J = 9.0, 4.4, 2.6 Hz, 1H), 7.55 (d, J = 2.2 Hz, 1H), 7.34 (dd, J = 10.6, 9.0 Hz, 1H), 7.02 (d, J = 2.1 Hz, 1H), 5.04 (hept, J = 6.2 Hz, 1H), 4.19 (s, 3H), 1.34 (d, J = 6.2 Hz, 6H). LCMS m/z = 438.2 (M + 1); RT = 1.76, Method 3. |
| 75 | 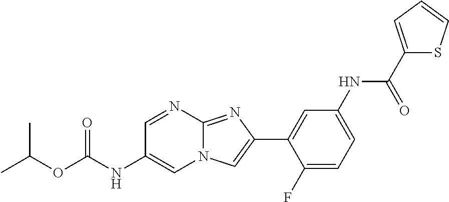 [SMILES: CC(C)OC(=O)Nc1cnc2nc(-c3cc(NC(=O)c4cccs4)ccc3F)cn2c1] | ¹H NMR (400 MHz, CD₃OD) δ 9.43 (s, 1H), 8.65 (d, J = 2.5 Hz, 1H), 8.33 (d, J = 3.0 Hz, 1H), 8.25 (dd, J = 6.7, 2.6 Hz, 1H), 7.93 (dd, J = 3.9, 1.1 Hz, 1H), 7.84-7.75 (m, 1H), 7.71 (dd, J = 5.1, 1.2 Hz, 1H), 7.28 (dd, J = 10.6, 9.0 Hz, 1H), 7.19 (dd, J = 5.0, 3.8 Hz, 1H), 5.03 (hept, J = 6.1 Hz, 1H), 1.34 (d, J = 6.3 Hz, 6H). LCMS m/z = 440 (M + 1); RT = 0.98, Method 2. |
| 76 | 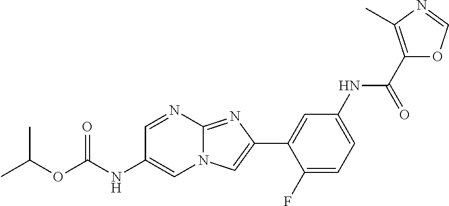 [SMILES: Cc1ncoc1C(=O)Nc1ccc(F)c(-c2cn3cc(NC(=O)OC(C)C)cnc3n2)c1] | ¹H NMR (400 MHz, CD₃OD) δ 9.49 (s, 1H), 8.69 (d, J = 2.6 Hz, 1H), 8.40 (d, J = 2.9 Hz, 1H), 8.32 (dd, J = 6.6, 2.7 Hz, 1H), 8.29 (s, 1H), 7.78 (ddd, J = 8.9, 4.4, 2.7 Hz, 1H), 7.34 (dd, J = 10.7, 9.0 Hz, 1H), 5.04 (hept, J = 6.0 Hz, 1H), 2.53 (s, 3H), 1.34 (d, J = 6.3 Hz, 6H). LCMS m/z = 439.4 (M + 1); RT = 1.54, Method 3. |
| 77 | 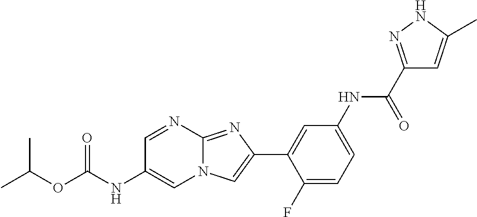 [SMILES: Cc1cc(C(=O)Nc2ccc(F)c(-c3cn4cc(NC(=O)OC(C)C)cnc4n3)c2)n[nH]1] | LCMS m/z = 438.2 (M + 1); RT = 1.66, Method 3. |
| 78 | 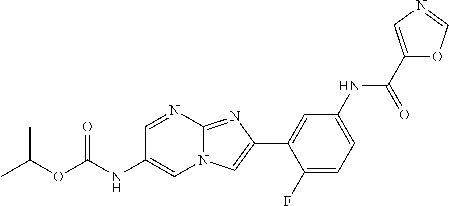 [SMILES: CC(C)OC(=O)Nc1cnc2nc(-c3cc(NC(=O)c4cnco4)ccc3F)cn2c1] | ¹H NMR (400 MHz, CD₃OD) δ 9.48 (s, 1H), 8.69 (d, J = 2.6 Hz, 1H), 8.44 (s, 1H), 8.39 (d, J = 2.9 Hz, 1H), 8.33 (dd, J = 6.5, 2.7 Hz, 1H), 7.92 (s, 1H), 7.80 (ddd, J = 8.9, 4.3, 2.7 Hz, 1H), 7.34 (dd, J = 10.6, 9.0 Hz, 1H), 5.04 (hept, J = 6.3 Hz, 1H), 1.34 (d, J = 6.3 Hz, 6H). LCMS m/z = 425.4 (M + 1); RT = 1.5, Method 3. |
| 79 | 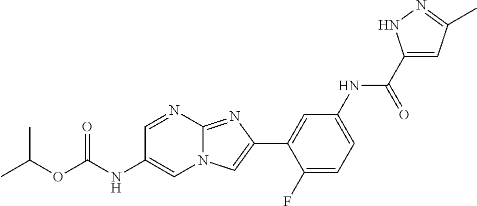 [SMILES: Cc1cc(C(=O)Nc2ccc(F)c(-c3cn4cc(NC(=O)OC(C)C)cnc4n3)c2)[nH]n1] | LCMS m/z = 438 (M + 1); RT = 0.87, Method 2. |
| 80 | 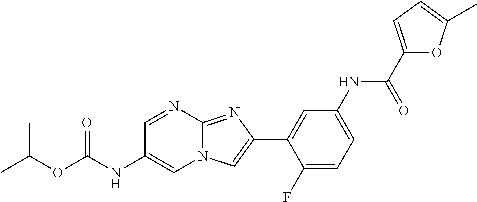 [SMILES: Cc1ccc(C(=O)Nc2ccc(F)c(-c3cn4cc(NC(=O)OC(C)C)cnc4n3)c2)o1] | LCMS m/z = 438.4 (M + 1); RT = 1.65, Method 3. |
| 81 | 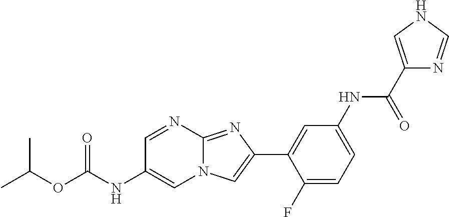 [SMILES: CC(C)OC(=O)Nc1cnc2nc(-c3cc(NC(=O)c4c[nH]cn4)ccc3F)cn2c1] | ¹H NMR (400 MHz, CD₃OD) δ 9.56 (s, 1H), 8.76 (d, J = 2.5 Hz, 1H), 8.45 (d, J = 2.6 Hz, 1H), 8.35 (dd, J = 6.5, 2.6 Hz, 1H), 7.86 (ddd, J = 8.8, 4.3, 2.7 Hz, 1H), 7.42-7.30 (m, 3H), 5.05 (hept, J = 6.2 Hz, 1H), 1.35 (d, J = 6.3 Hz, 6H). m/z = 424 (M + 1); RT = 0.83, Method 2. |
| 82 | 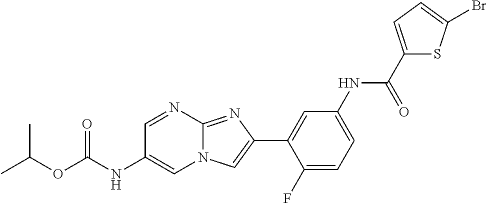 [SMILES: CC(C)OC(=O)Nc1cnc2nc(-c3cc(NC(=O)c4ccc(Br)s4)ccc3F)cn2c1] | LCMS m/z = 517.8 (M + 1); RT = 1.13, Method 2. |
| 83 | 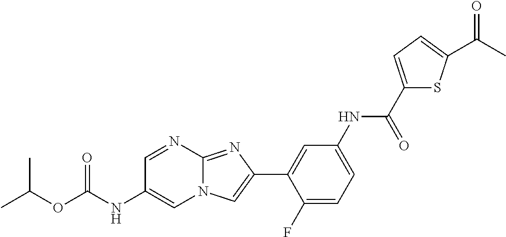 [SMILES: CC(=O)c1ccc(C(=O)Nc2ccc(F)c(-c3cn4cc(NC(=O)OC(C)C)cnc4n3)c2)s1] | LCMS m/z = 481.9 (M + 1); RT = 0.98, Method 2. |
| 84 | 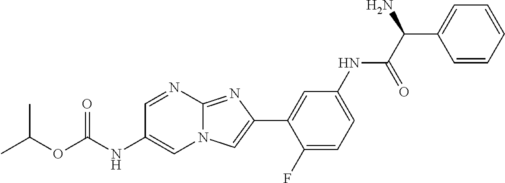 [SMILES: CC(C)OC(=O)Nc1cnc2nc(-c3cc(NC(=O)[C@@H](N)c4ccccc4)ccc3F)cn2c1] | LCMS m/z = 463.2 (M + 1); RT = 2.57, Method 1. |
| 85 | 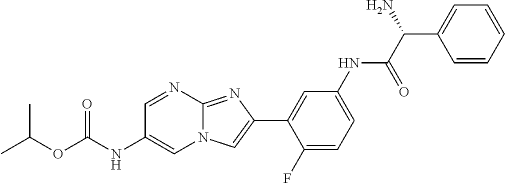 [SMILES: CC(C)OC(=O)Nc1cnc2nc(-c3cc(NC(=O)[C@H](N)c4ccccc4)ccc3F)cn2c1] | LCMS m/z = 463 (M + 1); RT = 0.74, Method 2. |
| 86 | 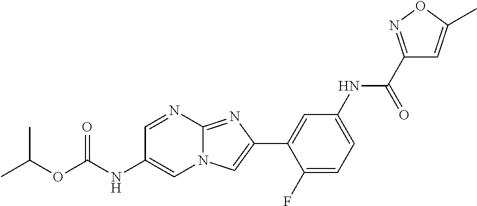 [SMILES: Cc1cc(C(=O)Nc2ccc(F)c(-c3cn4cc(NC(=O)OC(C)C)cnc4n3)c2)no1] | ¹H NMR (400 MHz, CD₃OD) δ 9.38 (s, 1H), 8.60 (d, J = 2.6 Hz, 1H), 8.30 (d, J = 3.3 Hz, 1H), 8.27 (dd, J = 6.5, 2.7 Hz, 1H), 7.87 (ddd, J = 8.9, 4.4, 2.8 Hz, 1H), 7.29 (dd, J = 10.7, 9.0 Hz, 1H), 6.57 (s, 1H), 5.03 (hept, J = 6.1 Hz, 1H), 2.52 (s, 3H), 1.34 (d, J = 6.3 Hz, 6H). LCMS m/z = 439 (M + 1); RT = 0.95, Method 2. |
| 87 | 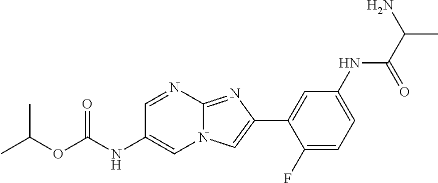 [SMILES: CC(C)OC(=O)Nc1cnc2nc(-c3cc(NC(=O)C(C)N)ccc3F)cn2c1] | LCMS m/z = 401 (M + 1); RT = 0.65, Method 2. |
| 88 | 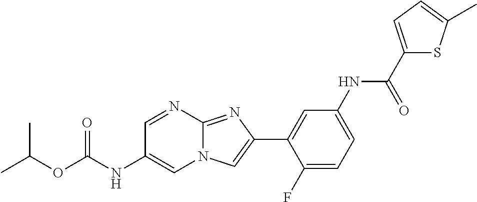 [SMILES: Cc1ccc(C(=O)Nc2ccc(F)c(-c3cn4cc(NC(=O)OC(C)C)cnc4n3)c2)s1] | LCMS m/z = 454.2 (M + 1); RT = 3.29, Method 1. |
| 89 | 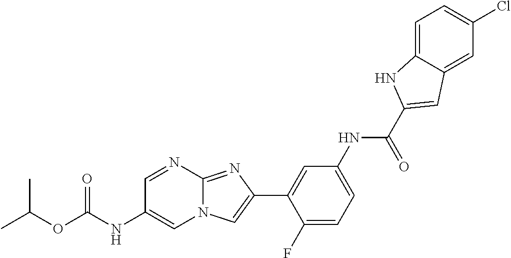 [SMILES: CC(C)OC(=O)Nc1cnc2nc(-c3cc(NC(=O)c4cc5cc(Cl)ccc5[nH]4)ccc3F)cn2c1] | ¹H NMR (400 MHz, CD₃OD) δ 9.49 (s, 1H), 8.70 (d, J = 2.5 Hz, 1H), 8.47-8.34 (m, 2H), 7.82 (d, J = 2.4 Hz, 1H), 7.66- 7.61 (m, 1H), 7.55-7.50 (m, 1H), 7.46- 7.33 (m, 2H), 7.26-7.17 (m, 1H), 5.09- 4.99 (m, 1H), 1.41-1.29 (m, 6H). LCMS m/z = 507.1 (M + 1); RT = 3.52, Method 1. |
| 90 | 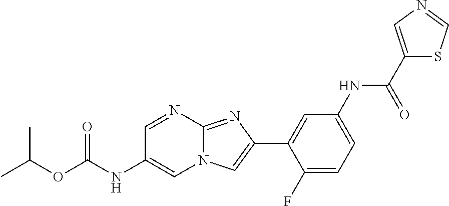 [SMILES: CC(C)OC(=O)Nc1cnc2nc(-c3cc(NC(=O)c4cncs4)ccc3F)cn2c1] | ¹H NMR (400 MHz, CD₃OD) δ 9.44 (s, 1H), 9.18 (s, 1H), 8.66 (d, J = 2.6 Hz, 1H), 8.64 (s, 1H), 8.35 (d, J = 3.0 Hz, 1H), 8.29 (dd, J = 6.5, 2.7 Hz, 1H), 7.84-7.81 (m, 1H), 7.31 (dd, J = 10.7, 9.0 Hz, 1H), 5.04 (hept, J = 6.1 Hz, 1H), 1.34 (d, J = 6.3 Hz, 6H). LCMS m/z = 441 (M + 1); RT = 0.9, Method 2. |
| 91 | 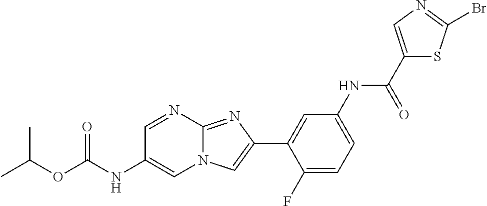 [SMILES: CC(C)OC(=O)Nc1cnc2nc(-c3cc(NC(=O)c4cnc(Br)s4)ccc3F)cn2c1] | ¹H NMR (400 MHz, CD₃OD) δ 9.50 (s, 1H), 8.70 (d, J = 2.5 Hz, 1H), 8.40 (d, J = 2.8 Hz, 1H), 8.28 (dd, J = 6.5, 2.7 Hz, 1H), 7.74 (ddd, J = 8.8, 4.3, 2.7 Hz, 1H), 7.64 (s, 1H), 7.33 (dd, J = 10.6, 9.0 Hz, 1H), 5.04 (hept, J = 6.2 Hz, 1H), 1.34 (d, J = 6.2 Hz, 6H). LCMS m/z = 518.8 (M + 1), 520.8 (M + 3). RT = 3.42, Method 1. |
| 92 | 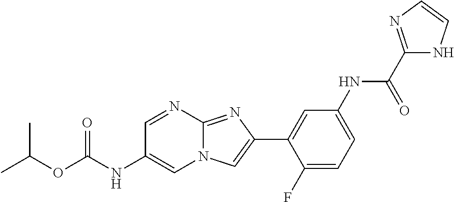 [SMILES: CC(C)OC(=O)Nc1cnc2nc(-c3cc(NC(=O)c4ncc[nH]4)ccc3F)cn2c1] | ¹H NMR (400 MHz, CD₃OD) δ 9.32 (s, 1H), 8.55 (d, J = 2.6 Hz, 1H), 8.24 (d, J = 3.4 Hz, 1H), 8.15 (dd, J = 6.5, 2.7 Hz, 1H), 7.85 (ddd, J = 8.9, 4.4, 2.8 Hz, 1H), 7.72 (d, J = 4.0 Hz, 1H), 7.23 (dd, J = 10.7, 9.0 Hz, 1H), 7.18 (d, J = 4.1 Hz, 1H), 5.02 (hept, J = 6.2 Hz, 1H), 1.33 (d, J = 6.3 Hz, 6H). LCMS m/z = 424.2 (M + 1); RT = 2.8, Method 1. |
| 93 | 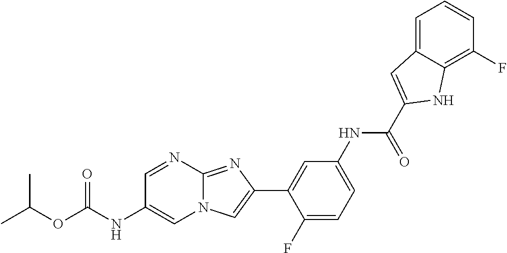 [SMILES: CC(C)OC(=O)Nc1cnc2nc(-c3cc(NC(=O)c4cc5cccc(F)c5[nH]4)ccc3F)cn2c1] | LCMS m/z = 491.2 (M + 1); RT = 3.43, Method 1. |
| 94 | 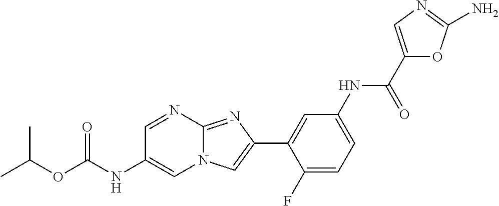 [SMILES: CC(C)OC(=O)Nc1cnc2nc(-c3cc(NC(=O)c4cnc(N)o4)ccc3F)cn2c1] | LCMS m/z = 440 (M + 1); RT = 0.8, Method 2. |
| 95 | 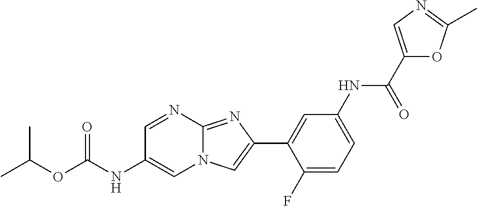 [SMILES: Cc1ncc(C(=O)Nc2ccc(F)c(-c3cn4cc(NC(=O)OC(C)C)cnc4n3)c2)o1] | LCMS m/z = 439.2 (M + 1); RT = 3, Method 1. |
| 96 | 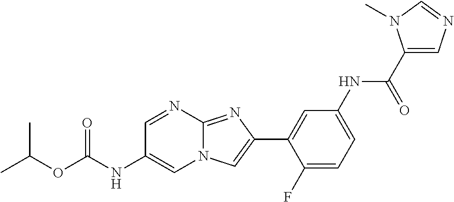 [SMILES: CC(C)OC(=O)Nc1cnc2nc(-c3cc(NC(=O)c4cncn4C)ccc3F)cn2c1] | LCMS m/z = 438 (M + 1); RT = 0.92, Method 2. |
| 97 | 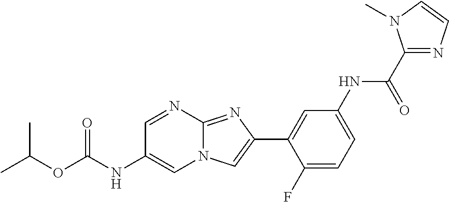 [SMILES: CC(C)OC(=O)Nc1cnc2nc(-c3cc(NC(=O)c4nccn4C)ccc3F)cn2c1] | LCMS m/z = 438 (M + 1); RT = 0.72, Method 2. |
| 98 | 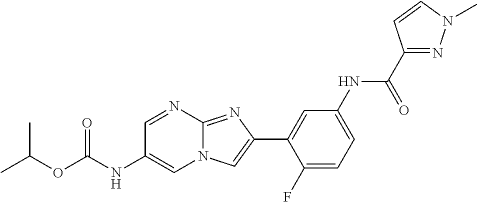 [SMILES: CC(C)OC(=O)Nc1cnc2nc(-c3cc(NC(=O)c4ccn(C)n4)ccc3F)cn2c1] | LCMS m/z = 438 (M + 1); RT = 0.89, Method 2. |
| 99 | 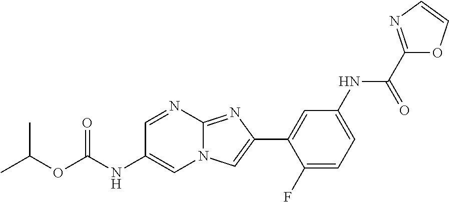 [SMILES: CC(C)OC(=O)Nc1cnc2nc(-c3cc(NC(=O)c4ncco4)ccc3F)cn2c1] | LCMS m/z = 424.9 (M + 1); RT = 0.92, Method 2. |
| 100 | 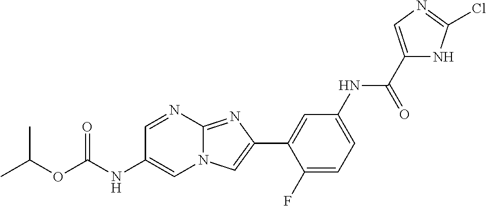 [SMILES: CC(C)OC(=O)Nc1cnc2nc(-c3cc(NC(=O)c4cnc(Cl)[nH]4)ccc3F)cn2c1] | LCMS m/z = 457.9 (M + 1); RT = 0.87, Method 2. |
| 101 | 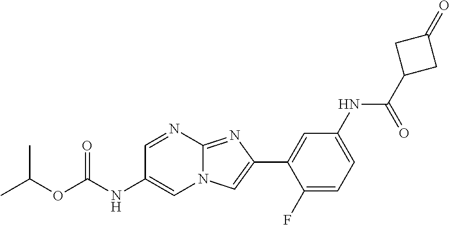 [SMILES: CC(C)OC(=O)Nc1cnc2nc(-c3cc(NC(=O)C4CC(=O)C4)ccc3F)cn2c1] | ¹H NMR (400 MHz, CD₃OD) δ 9.52 (s, 1H), 8.73 (d, J = 2.6 Hz, 1H), 8.41 (d, J = 2.7 Hz, 1H), 8.30 (dd, J = 6.5, 2.7 Hz, 1H), 7.65 (ddd, J = 9.0, 4.4, 2.8 Hz, 1H), 7.32 (dd, J = 10.6, 9.0 Hz, 1H), 5.05 (hept, J = 6.1 Hz, 1H), 3.47- 3.34 (m, 5H), 1.34 (d, J = 6.2 Hz, 6H). LCMS m/z = 426 (M + 1); RT = 0.87, Method 2. |
| 102 | 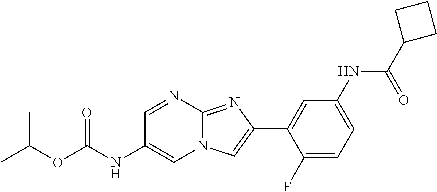 [SMILES: CC(C)OC(=O)Nc1cnc2nc(-c3cc(NC(=O)C4CCC4)ccc3F)cn2c1] | ¹H NMR (400 MHz, CD₃OD) δ 9.52 (s, 1H), 8.73 (d, J = 2.6 Hz, 1H), 8.40 (d, J = 2.7 Hz, 1H), 8.23 (dd, J = 6.5, 2.7 Hz, 1H), 7.62 (ddd, J = 9.0, 4.4, 2.6 Hz, 1H), 7.29 (dd, J = 10.6, 9.0 Hz, 1H), 5.05 (hept, J = 6.2 Hz, 1H), 3.33- 3.29 (m, 1H), 2.38 (dq, J = 11.5, 9.1 Hz, 2H), 2.30-2.19 (m, 2H), 2.15-1.99 (m, 1H), 1.99-1.87 (m, 1H), 1.34 (d, J = 6.2 Hz, 6H). LCMS m/z = 412.2 (M + 1); RT = 3.19, Method 1. |
| 103 | 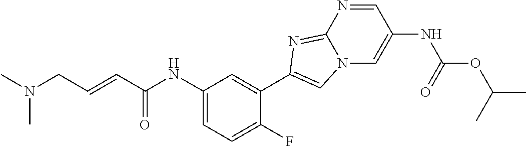 [SMILES: CC(C)OC(=O)Nc1cnc2nc(-c3cc(NC(=O)/C=C/CN(C)C)ccc3F)cn2c1] | ¹H NMR (400 MHz, CD₃OD): 9.49 (s, 1H), 8.70 (dd, J = 5.0, 2.6 Hz, 1H), 8.39 (d, J = 2.8 Hz, 1H), 8.33-8.19 (m, 1H), 7.66 (ddd, J = 8.9, 4.4, 2.6 Hz, 1H), 7.40-7.15 (m, 1H), 5.13-4.99 (m, 1H), 3.26 (s, 1H), 2.99 (dd, J = 9.6, 7.9 Hz, 1H), 2.64-1.79 (m, 8H), 1.35 (d, J = 6.2 Hz, 6H). |
| 104 | 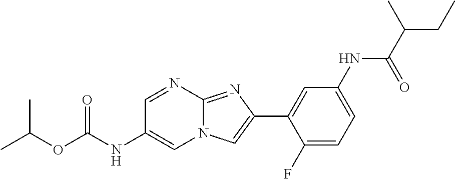 [SMILES: CCC(C)C(=O)Nc1ccc(F)c(-c2cn3cc(NC(=O)OC(C)C)cnc3n2)c1] | ¹H NMR (400 MHz, CD₃OD) δ 9.52 (s, 1H), 8.25 (dd, J = 6.6, 2.6 Hz, 1H), 7.66 (ddd, J = 8.9, 4.4, 2.7 Hz, 1H), 7.53 (dd, J = 8.5, 4.5 Hz, 2H), 7.31 (dd, J = 10.6, 9.1 Hz, 1H), 5.05 (hept, J = 6.3 Hz, 1H), 2.53-2.37 (m, 1H), 1.83- 1.64 (m, 1H), 1.60-1.46 (m, 1H), 1.35 (d, J = 6.2 Hz, 6H), 1.22 (d, J = 6.8 Hz, 3H), 0.99 (t, J = 7.4 Hz, 3H). LCMS m/z = 414.2 (M + 1); RT = 3.22, Method 1. |
| 105 | 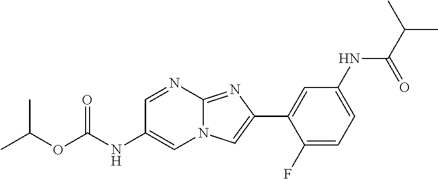 [SMILES: CC(C)OC(=O)Nc1cnc2nc(-c3cc(NC(=O)C(C)C)ccc3F)cn2c1] | ¹H NMR (400 MHz, CD₃OD) δ 9.52 (s, 1H), 8.72 (d, J = 2.6 Hz, 1H), 8.40 (d, J = 2.7 Hz, 1H), 8.23 (dd, J = 6.6, 2.6 Hz, 1H), 7.64 (ddd, J = 8.9, 4.4, 2.7 Hz, 1H), 7.30 (dd, J = 10.7, 9.0 Hz, 1H), 5.05 (hept, J = 6.1 Hz, 1H), 2.66 (hept, J = 6.8 Hz, 1H), 1.34 (d, J = 6.3 Hz, 6H), 1.23 (d, J = 6.9 Hz, 6H). LCMS m/z = 400.2 (M + 1); RT = 3.12, Method 1. |
| 106 | 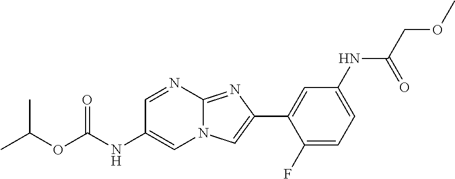 [SMILES: COCC(=O)Nc1ccc(F)c(-c2cn3cc(NC(=O)OC(C)C)cnc3n2)c1] | ¹H NMR (400 MHz, CD₃OD) δ 9.55- 9.45 (m, 1H), 8.71 (d, J = 2.6 Hz, 1H), 8.40 (d, J = 2.8 Hz, 1H), 8.26 (dd, J = 6.7, 2.7 Hz, 1H), 7.70 (ddd, J = 8.9, 4.4, 2.7 Hz, 1H), 7.32 (dd, J = 10.6, 9.0 Hz, 1H), 5.04 (hept, J = 6.1 Hz, 1H), 4.09 (s, 2H), 3.52 (s, 3H), 1.34 (d, J = 6.3 Hz, 6H). LCMS m/z = 402.2 (M + 1); RT = 2.91, Method 1. |
| 107 | 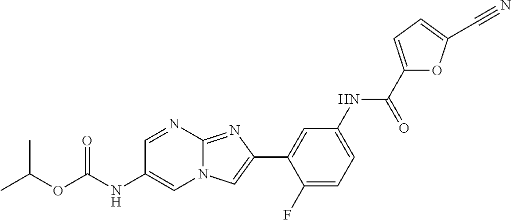 [SMILES: CC(C)OC(=O)Nc1cnc2nc(-c3cc(NC(=O)c4ccc(C#N)o4)ccc3F)cn2c1] | ¹H NMR (400 MHz, CD₃OD) δ 9.33 (s, 1H), 8.55 (dd, J = 7.0, 2.6 Hz, 1H), 8.30-8.18 (m, 1H), 7.97 (s, 1H), 7.90 (ddd, J = 8.9, 4.4, 2.8 Hz, 1H), 7.43 (dd, J = 20.0, 3.7 Hz, 2H), 7.33-7.19 (m, 1H), 5.09-4.96 (m, 1H), 1.33 (d, J = 6.8 Hz, 6H). LCMS m/z = 448.9 (M + 1); RT = 0.98, Method 2. |
| 108 | 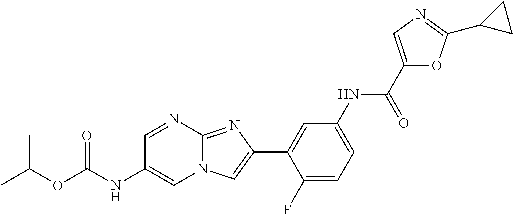 [SMILES: CC(C)OC(=O)Nc1cnc2nc(-c3cc(NC(=O)c4cnc(C5CC5)o4)ccc3F)cn2c1] | ¹H NMR (400 MHz, CD₃OD) δ 9.41 (s, 1H), 8.74 (d, J = 2.6 Hz, 1H), 8.49 (s, 1H), 8.33 (d, J = 3.3 Hz, 1H), 7.76 (s, 1H), 7.55-7.42 (m, 1H), 7.30 (dd, J = 10.7, 9.0 Hz, 1H), 5.11-4.96 (m, 1H), 3.05 (d, J = 6.8 Hz, 1H), 1.35 (dd, J = 6.2, 1.6 Hz, 6H), 1.21 (dd, J = 7.7, 3.6 Hz, 2H), 0.96 (d, J = 6.7 Hz, 2H). LCMS m/z = 465.4 (M + 1); RT = 1.61, Method 3. |
| 109 | 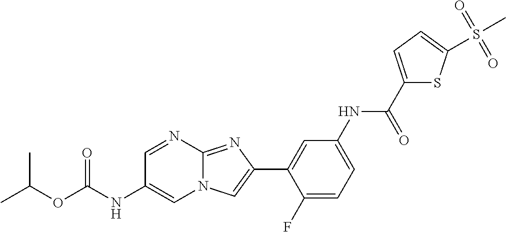 [SMILES: CC(C)OC(=O)Nc1cnc2nc(-c3cc(NC(=O)c4ccc(S(C)(=O)=O)s4)ccc3F)cn2c1] | ¹H NMR (400 MHz, CD₃OD) δ 9.54 (s, 1H), 8.73 (d, J = 2.6 Hz, 1H), 8.45 (d, J = 2.7 Hz, 1H), 8.39 (dd, J = 6.5, 2.7 Hz, 1H), 7.97 (d, J = 4.1 Hz, 1H), 7.82 (d, J = 4.0 Hz, 1H), 7.79 (ddd, J = 8.9, 4.3, 2.7 Hz, 1H), 7.39 (dd, J = 10.6, 9.0 Hz, 1H), 5.05 (hept, J = 6.2 Hz, 1H), 4.88 (s, 3H), 1.35 (d, J = 6.2 Hz, 6H). LCMS m/z = 518 (M + 1); RT = 0.98, Method 2. |
| 110 | 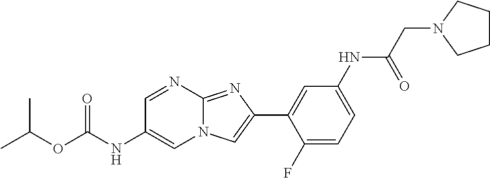 [SMILES: CC(C)OC(=O)Nc1cnc2nc(-c3cc(NC(=O)CN4CCCC4)ccc3F)cn2c1] | ¹H NMR (400 MHz, CD₃OD) δ 9.51 (s, 1H), 8.73 (d, J = 2.6 Hz, 1H), 8.41 (d, J = 2.7 Hz, 1H), 8.25 (dd, J = 6.5, 2.7 Hz, 1H), 7.71 (ddd, J = 8.9, 4.3, 2.6 Hz, 1H), 7.33 (dd, J = 10.6, 9.0 Hz, 1H), 5.04 (hept, J = 6.3 Hz, 1H), 4.29 (s, 2H), 3.81 (bs, 2H), 3.24 (bs, 2H), 2.15 (bd, J = 22.9 Hz, 4H), 1.34 (d, J = 6.3 Hz, 6H). LCMS m/z = 441 (M + 1); RT = 0.68, Method 2. |
| 111 | 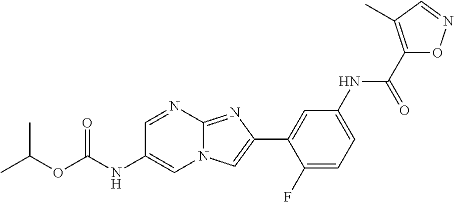 [SMILES: Cc1cnoc1C(=O)Nc1ccc(F)c(-c2cn3cc(NC(=O)OC(C)C)cnc3n2)c1] | LCMS m/z = 439 (M + 1); RT = 0.96, Method 2. |
| 112 | 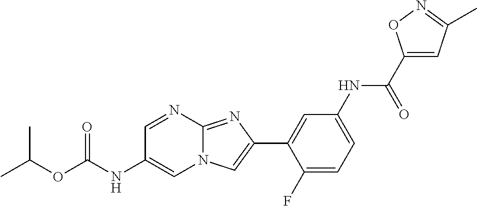 [SMILES: Cc1cc(C(=O)Nc2ccc(F)c(-c3cn4cc(NC(=O)OC(C)C)cnc4n3)c2)on1] | LCMS m/z = 439 (M + 1); RT = 0.93, Method 2. |
| 113 | 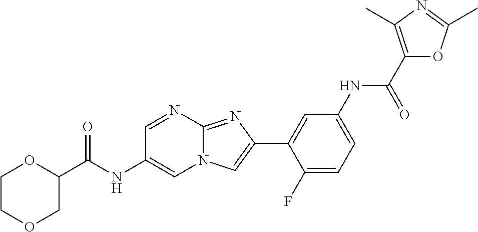 [SMILES: Cc1nc(C)c(C(=O)Nc2ccc(F)c(-c3cn4cc(NC(=O)C5COCCO5)cnc4n3)c2)o1] | ¹H NMR (400 MHz, CD₃OD) δ 9.66 (d, J = 2.4 Hz, 1H), 8.87 (d, J = 2.5 Hz, 1H), 8.34 (d, J = 2.9 Hz, 1H), 8.28 (dd, J = 6.6, 2.7 Hz, 1H), 7.75 (ddd, J = 8.9, 4.4, 2.7 Hz, 1H), 7.28 (dd, J = 10.7, 9.0 Hz, 1H), 4.34 (dd, J = 9.7, 3.2 Hz, 1H), 4.09 (dd, J = 11.6, 3.2 Hz, 1H), 4.00 (dt, J = 11.8, 2.1 Hz, 1H), 3.86 (td, J = 11.6, 11.2, 2.7 Hz, 1H), 3.79 (dt, J = 11.8, 2.2 Hz, 1H), 3.72-3.57 (m, 2H), 2.55 (s, 3H), 2.47 (s, 3H). LCMS m/z = 481.4 (M + 1); RT = 1.44, Method 3. |
| 114 | 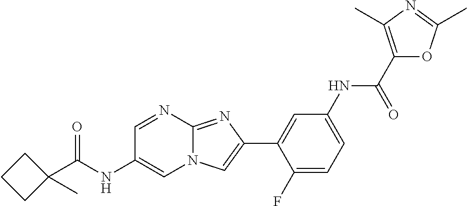 [SMILES: Cc1nc(C)c(C(=O)Nc2ccc(F)c(-c3cn4cc(NC(=O)C5(C)CCC5)cnc4n3)c2)o1] | ¹H NMR (400 MHz, CD₃OD) δ 9.77 (d, J = 2.5 Hz, 1H), 8.88 (d, J = 2.5 Hz, 1H), 8.44 (d, J = 2.8 Hz, 1H), 8.34 (dd, J = 6.6, 2.7 Hz, 1H), 7.75 (ddd, J = 9.0, 4.4, 2.7 Hz, 1H), 7.34 (dd, J = 10.6, 8.9 Hz, 1H), 2.63-2.52 (m, 5H), 2.47 (s, 3H), 2.13-1.93 (m, 3H), 1.90-1.78 (m, 1H), 1.56 (s, 3H). LCMS m/z = 463.4 (M + 1); RT = 1.61, Method 3. |
| 115 | 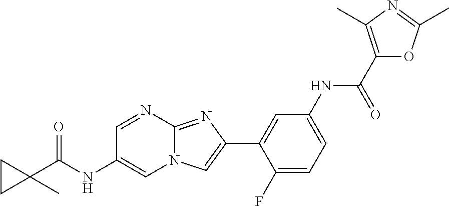 [SMILES: Cc1nc(C)c(C(=O)Nc2ccc(F)c(-c3cn4cc(NC(=O)C5(C)CC5)cnc4n3)c2)o1] | ¹H NMR (400 MHz, CD₃OD) δ 9.70 (d, J = 2.5 Hz, 1H), 8.98 (d, J = 2.4 Hz, 1H), 8.74 (dd, J = 4.4, 1.0 Hz, 1H), 8.35 (dd, J = 6.6, 2.6 Hz, 1H), 7.75 (ddd, J = 8.9, 4.4, 2.7 Hz, 1H), 7.35 (dd, J = 10.5, 9.1 Hz, 1H), 2.56 (s, 3H), 2.48 (s, 3H), 1.53 (s, 3H), 1.29 (q, J = 3.9 Hz, 2H), 0.82 (q, J = 4.0 Hz, 2H). LCMS m/z = 449.4 (M + 1); RT = 1.55, Method 3. |
| 116 | 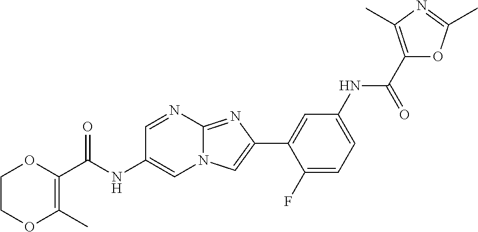 [SMILES: CC1=C(C(=O)Nc2cnc3nc(-c4cc(NC(=O)c5oc(C)nc5C)ccc4F)cn3c2)OCCO1] | ¹H NMR (400 MHz, CD₃OD) δ 9.87 (d, J = 2.5 Hz, 1H), 9.06 (d, J = 2.4 Hz, 1H), 8.48 (d, J = 2.4 Hz, 1H), 8.37 (dd, J = 6.5, 2.7 Hz, 1H), 7.74 (ddd, J = 8.9, 4.4, 2.6 Hz, 1H), 7.38 (dd, J = 10.6, 8.9 Hz, 1H), 4.25 (dd, J = 5.4, 2.8 Hz, 2H), 4.19 (dd, J = 5.3, 2.7 Hz, 2H), 2.56 (s, 3H), 2.48 (s, 3H), 2.30 (s, 3H). LCMS m/z = 493.4 (M + 1); RT = 1.63, Method 3. |
| 117 | 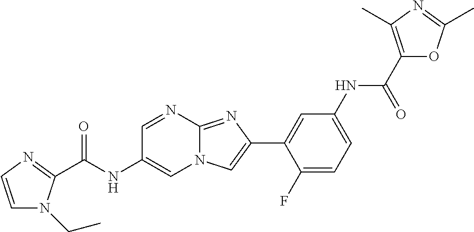 [SMILES: CCn1ccnc1C(=O)Nc1cnc2nc(-c3cc(NC(=O)c4oc(C)nc4C)ccc3F)cn2c1] | ¹H NMR (400 MHz, CD₃OD) δ 9.76 (d, J = 2.7 Hz, 1H), 8.92 (d, J = 2.6 Hz, 1H), 8.31 (d, J = 3.0 Hz, 1H), 8.20 (dd, J = 6.4, 2.7 Hz, 1H), 7.83 (ddd, J = 8.9, 4.6, 2.8 Hz, 1H), 7.32 (d, J = 1.1 Hz, 1H), 7.28-7.22 (m, 1H), 7.15 (d, J = 1.1 Hz, 1H), 4.56 (q, J = 7.2 Hz, 2H), 2.56 (s, 3H), 2.48 (s, 3H), 1.49 (t, J = 7.3 Hz, 3H). LCMS m/z = 489.4 (M + 1); RT = 1.61, Method 3. |
| 118 | 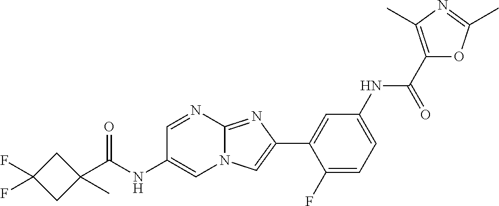 [SMILES: Cc1nc(C)c(C(=O)Nc2ccc(F)c(-c3cn4cc(NC(=O)C5(C)CC(F)(F)C5)cnc4n3)c2)o1] | ¹H NMR (400 MHz, CD₃OD) δ 9.70 (d, J = 2.5 Hz, 1H), 8.77 (d, J = 2.6 Hz, 1H), 8.38 (d, J = 3.1 Hz, 1H), 8.32 (dd, J = 6.7, 2.7 Hz, 1H), 7.78 (ddd, J = 8.9, 4.4, 2.7 Hz, 1H), 7.31 (dd, J = 10.7, 9.0 Hz, 1H), 3.24-3.07 (m, 2H), 2.66-2.50 (m, 5H), 2.47 (s, 3H), 1.64 (s, 3H). LCMS m/z = 499.4 (M + 1); RT = 1.65, Method 3. |
| 119 | 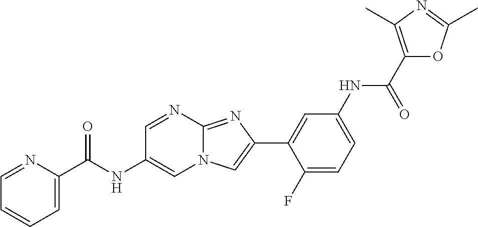 [SMILES: Cc1nc(C)c(C(=O)Nc2ccc(F)c(-c3cn4cc(NC(=O)c5ccccn5)cnc4n3)c2)o1] | ¹H NMR (400 MHz, CD₃OD) δ 9.89 (d, J = 2.5 Hz, 1H), 9.07 (d, J = 2.5 Hz, 1H), 8.76-8.69 (m, 1H), 8.37 (d, J = 2.9 Hz, 1H), 8.28-8.20 (m, 2H), 8.01 (td, J = 7.7, 1.7 Hz, 1H), 7.80 (ddd, J = 8.9, 4.4, 2.7 Hz, 1H), 7.62 (ddd, J = 7.6, 4.7, 1.3 Hz, 1H), 7.27 (dd, J = 10.6, 8.9 Hz, 1H), 2.56 (s, 3H), 2.48 (s, 3H). LCMS m/z = 472.4 (M + 1); RT = 1.63, Method 3. |
| 120 | 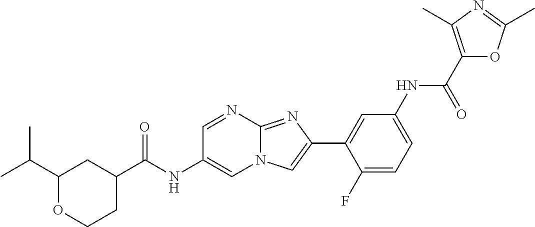 [SMILES: Cc1nc(C)c(C(=O)Nc2ccc(F)c(-c3cn4cc(NC(=O)C5CCOC(C(C)C)C5)cnc4n3)c2)o1] | ¹H NMR (400 MHz, CD₃OD) δ 9.77 (d, J = 2.5 Hz, 1H), 8.76 (d, J = 2.6 Hz, 1H), 8.41 (d, J = 2.8 Hz, 1H), 8.33 (dd, J = 6.6, 2.7 Hz, 1H), 7.75 (ddd, J = 8.9, 4.4, 2.7 Hz, 1H), 7.36-7.27 (m, 1H), 4.09 (dt, J = 11.4, 3.4 Hz, 1H), 3.60- 3.43 (m, 1H), 3.13 (ddd, J = 11.2, 6.4, 1.8 Hz, 1H), 2.82-2.62 (m, 1H), 2.56 (s, 3H), 2.48 (s, 3H), 1.97-1.86 (m, 1H), 1.84-1.64 (m, 3H), 1.53 (q, J = 12.1 Hz, 1H), 0.95 (ddd, J = 9.8, 8.5, 5.9 Hz, 6H). LCMS m/z = 521.4 (M + 1); RT = 1.66, Method 3. |
| 121 | 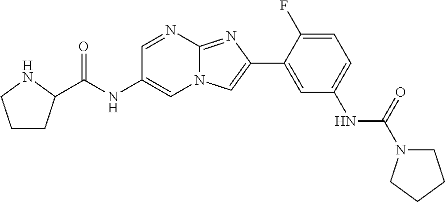 [SMILES: O=C(Nc1cnc2nc(-c3cc(NC(=O)N4CCCC4)ccc3F)cn2c1)C1CCCN1] | LCMS m/z = 438 (M + 1); RT = 0.51, Method 2. |
| 122 | 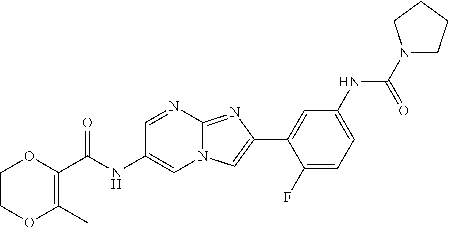 [SMILES: CC1=C(C(=O)Nc2cnc3nc(-c4cc(NC(=O)N5CCCC5)ccc4F)cn3c2)OCCO1] | LCMS m/z = 467 (M + 1); RT = 0.86, Method 2. |
| 123 | 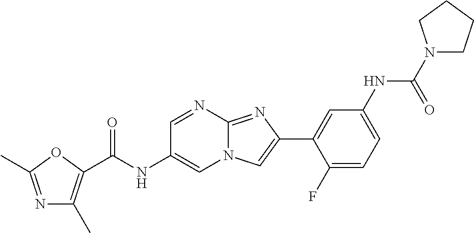 [SMILES: Cc1nc(C)c(C(=O)Nc2cnc3nc(-c4cc(NC(=O)N5CCCC5)ccc4F)cn3c2)o1] | ¹H NMR (400 MHz, CD₃OD) δ 9.77 (d, J = 2.6 Hz, 1H), 8.96 (d, J = 2.5 Hz, 1H), 8.36 (d, J = 3.0 Hz, 1H), 8.03 (dd, J = 6.6, 2.7 Hz, 1H), 7.64-7.42 (m, 1H), 7.22 (dd, J = 10.7, 9.0 Hz, 1H), 3.58- 3.44 (m, 4H), 2.57 (s, 3H), 2.49 (s, 3H), 2.01 (t, J = 6.5 Hz, 4H). LCMS m/z = 464.4 (M + 1); RT = 1.44, Method 3. |
| 124 | 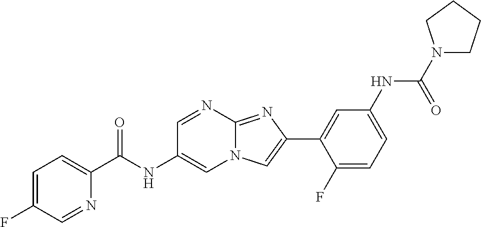 [SMILES: O=C(Nc1cnc2nc(-c3cc(NC(=O)N4CCCC4)ccc3F)cn2c1)c1ccc(F)cn1] | ¹H NMR (400 MHz, CD₃OD) δ 9.98 (d, J = 2.4 Hz, 1H), 9.16 (d, J = 2.4 Hz, 1H), 8.67 (d, J = 2.8 Hz, 1H), 8.46 (d, J = 2.7 Hz, 1H), 8.35 (dd, J = 8.7, 4.5 Hz, 1H), 8.06 (dd, J = 6.6, 2.7 Hz, 1H), 7.86 (td, J = 8.5, 2.8 Hz, 1H), 7.52 (ddd, J = 9.0, 4.5, 2.7 Hz, 1H), 7.27 (dd, J = 10.7, 9.0 Hz, 1H), 3.50 (td, J = 6.0, 5.3, 2.5 Hz, 4H), 2.01 (td, J = 8.2, 6.8, 4.9 Hz, 4H). LCMS m/z = 464.2 (M + 1); RT = 1.52, Method 3. |
| 125 | 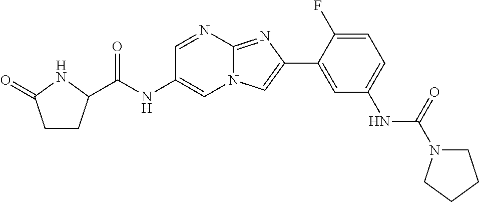 [SMILES: O=C1CCC(C(=O)Nc2cnc3nc(-c4cc(NC(=O)N5CCCC5)ccc4F)cn3c2)N1] | LCMS m/z = 452 (M + 1); RT = 0.65, Method 2. |
| 126 | 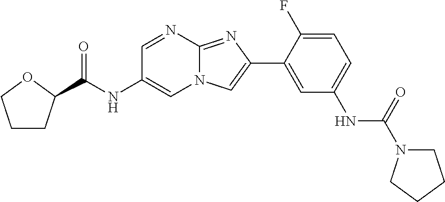 [SMILES: O=C(Nc1cnc2nc(-c3cc(NC(=O)N4CCCC4)ccc3F)cn2c1)[C@H]1CCCO1] | LCMS m/z = 439.3 (M + 1); RT = 1.31, Method 3. |
| 127 | 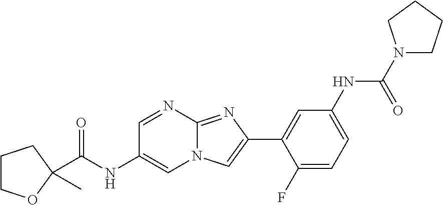 [SMILES: CC1(C(=O)Nc2cnc3nc(-c4cc(NC(=O)N5CCCC5)ccc4F)cn3c2)CCCO1] | ¹H NMR (400 MHz, CD₃OD) δ 9.84 (d, J = 2.4 Hz, 1H), 9.06 (d, J = 2.5 Hz, 1H), 8.44 (dd, J = 7.4, 1.9 Hz, 1H), 8.07 (dd, J = 6.6, 2.7 Hz, 1H), 7.53 (dd, J = 8.2, 4.4 Hz, 1H), 7.28 (dd, J = 10.6, 9.0 Hz, 1H), 4.15-3.98 (m, 2H), 3.56-3.45 (m, 4H), 1.98 (dt, J = 15.8, 5.4 Hz, 8H), 1.52 (s, 3H). LCMS m/z = 453.3 (M + 1); RT = 1.41, Method 3. |
| 128 | 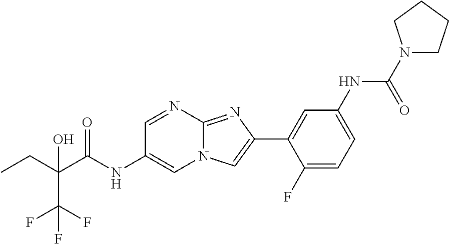 [SMILES: CCC(O)(C(=O)Nc1cnc2nc(-c3cc(NC(=O)N4CCCC4)ccc3F)cn2c1)C(F)(F)F] | ¹H NMR (400 MHz, CD₃OD) δ 9.68 (d, J = 2.5 Hz, 1H), 8.89 (d, J = 2.5 Hz, 1H), 8.34 (d, J = 3.2 Hz, 1H), 8.02 (dd, J = 6.6, 2.7 Hz, 1H), 7.55 (ddd, J = 8.9, 4.4, 2.8 Hz, 1H), 7.22 (dd, J = 10.8, 9.0 Hz, 1H), 3.58-3.43 (m, 4H), 2.38-2.21 (m, 1H), 2.00 (t, J = 6.5 Hz, 4H), 1.95- 1.84 (m, 1H), 1.02 (t, J = 7.4 Hz, 3H). LCMS m/z = 495.2 (M + 1); RT = 1.52, Method 3. |
| 129 | 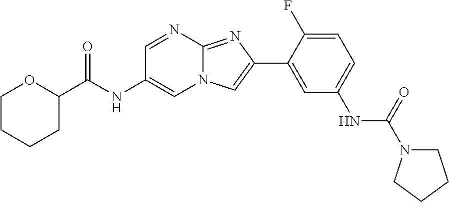 [SMILES: O=C(Nc1cnc2nc(-c3cc(NC(=O)N4CCCC4)ccc3F)cn2c1)C1CCCCO1] | LCMS m/z = 453.3 (M + 1); RT = 1.44, Method 3. |
| 130 | 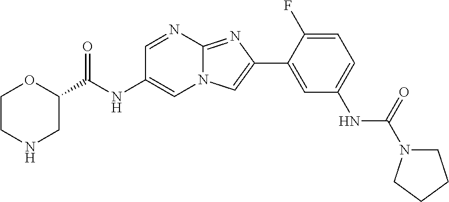 [SMILES: O=C(Nc1cnc2nc(-c3cc(NC(=O)N4CCCC4)ccc3F)cn2c1)[C@@H]1CNCCO1] | LCMS m/z = 454 (M + 1); RT = 0.56, Method 2. |
| 131 | 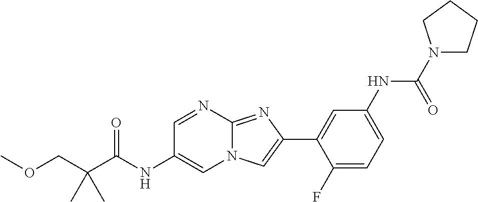 [SMILES: COCC(C)(C)C(=O)Nc1cnc2nc(-c3cc(NC(=O)N4CCCC4)ccc3F)cn2c1] | LCMS m/z = 453.3 (M + 1); RT = 1.41, Method 3. |
| 132 | 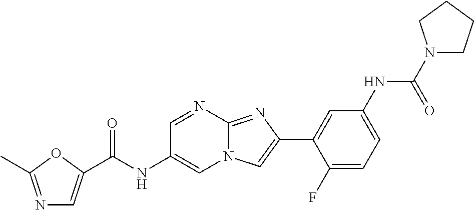 [SMILES: Cc1ncc(C(=O)Nc2cnc3nc(-c4cc(NC(=O)N5CCCC5)ccc4F)cn3c2)o1] | ¹H NMR (400 MHz, CD₃OD) δ 9.79 (d, J = 2.5 Hz, 1H), 8.94 (d, J = 2.5 Hz, 1H), 8.40 (d, J = 2.9 Hz, 1H), 8.04 (dd, J = 6.6, 2.7 Hz, 1H), 7.84 (s, 1H), 7.52 (ddd, J = 8.9, 4.4, 2.8 Hz, 1H), 7.24 (dd, J = 10.7, 9.0 Hz, 1H), 3.59-3.40 (m, 4H), 2.62 (s, 3H), 2.01 (t, J = 6.4 Hz, 4H). LCMS m/z = 450.4 (M + 1); RT = 1.41, Method 3. |
| 133 | 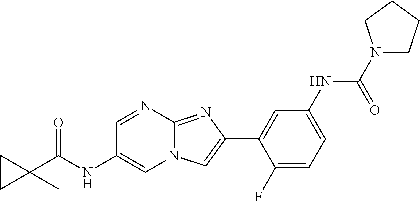 [SMILES: CC1(C(=O)Nc2cnc3nc(-c4cc(NC(=O)N5CCCC5)ccc4F)cn3c2)CC1] | ¹H NMR (400 MHz, CD₃OD) δ 9.68 (d, J = 2.5 Hz, 1H), 8.96 (d, J = 2.5 Hz, 1H), 8.39 (d, J = 2.7 Hz, 1H), 8.04 (dd, J = 6.6, 2.7 Hz, 1H), 7.57-7.42 (m, 1H), 7.36-7.11 (m, 1H), 3.55-3.43 (m, 4H), 2.00 (t, J = 6.4 Hz, 4H), 1.52 (s, 3H), 1.29 (q, J = 3.9 Hz, 2H), 0.81 (q, J = 4.0 Hz, 2H). LCMS m/z = 423.2 (M + 1); RT = 1.42, Method 3. |
| 134 | 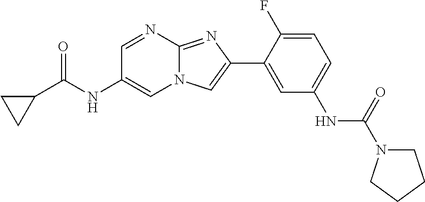 [SMILES: O=C(Nc1cnc2nc(-c3cc(NC(=O)N4CCCC4)ccc3F)cn2c1)C1CC1] | LCMS m/z = 409.2 (M + 1); RT = 1.37, Method 3. |
| 135 | 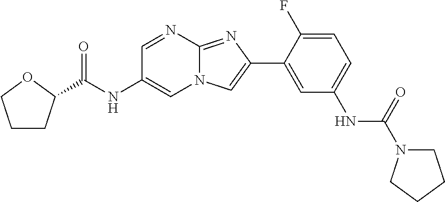 [SMILES: O=C(Nc1cnc2nc(-c3cc(NC(=O)N4CCCC4)ccc3F)cn2c1)[C@@H]1CCCO1] | LCMS m/z = 439.3 (M + 1); RT = 1.31, Method 3. |
| 136 | 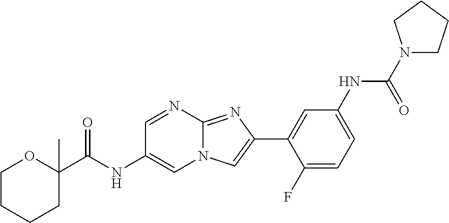 [SMILES: CC1(C(=O)Nc2cnc3nc(-c4cc(NC(=O)N5CCCC5)ccc4F)cn3c2)CCCCO1] | ¹H NMR (400 MHz, CD₃OD) δ 9.86 (d, J = 2.4 Hz, 1H), 9.05 (d, J = 2.5 Hz, 1H), 8.45 (d, J = 2.5 Hz, 1H), 8.06 (dd, J = 6.6, 2.7 Hz, 1H), 7.50 (ddd, J = 8.9, 4.4, 2.7 Hz, 1H), 7.28 (dd, J = 10.6, 9.0 Hz, 1H), 4.01-3.83 (m, 1H), 3.75-3.64 (m, 1H), 3.55-3.43 (m, 4H), 2.25-2.12 (m, 1H), 2.01 (t, J = 6.5 Hz, 4H), 1.78 (d, J = 4.3 Hz, 1H), 1.58 (d, J = 11.1 Hz, 4H), 1.48 (s, 3H). LCMS m/z = 467.3 (M + 1); RT = 1.5, Method 3. |
| 137 | 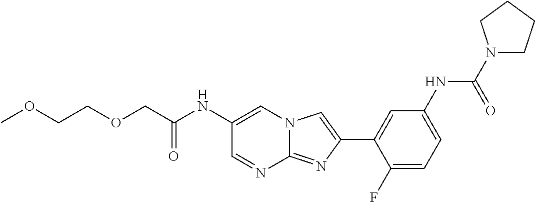 [SMILES: COCCOCC(=O)Nc1cnc2nc(-c3cc(NC(=O)N4CCCC4)ccc3F)cn2c1] | LCMS m/z = 457.3 (M + 1); RT = 1.31, Method 3. |
| 138 | 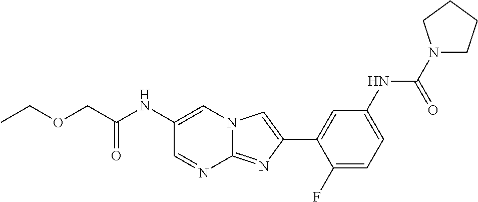 [SMILES: CCOCC(=O)Nc1cnc2nc(-c3cc(NC(=O)N4CCCC4)ccc3F)cn2c1] | ¹H NMR (400 MHz, CD₃OD) δ 9.76 (d, J = 2.4 Hz, 1H), 8.93 (d, J = 2.5 Hz, 1H), 8.40 (d, J = 2.8 Hz, 1H), 8.04 (dd, J = 6.6, 2.7 Hz, 1H), 7.52 (ddd, J = 8.9, 4.4, 2.7 Hz, 1H), 7.25 (dd, J = 10.6, 9.0 Hz, 1H), 4.20 (s, 2H), 3.71 (q, J = 7.0 Hz, 2H), 3.58-3.40 (m, 4H), 2.00 (s, 4H), 1.33 (t, J = 7.0 Hz, 3H). LCMS m/z = 427.2 (M + 1); RT = 1.31, Method 3. |
| 139 | 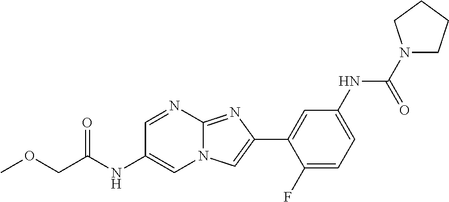 [SMILES: COCC(=O)Nc1cnc2nc(-c3cc(NC(=O)N4CCCC4)ccc3F)cn2c1] | ¹H NMR (400 MHz, CD₃OD) δ 9.85 (s, 1H), 9.01 (d, J = 2.5 Hz, 1H), 8.46 (d, J = 2.5 Hz, 1H), 8.07 (dd, J = 6.6, 2.7 Hz, 1H), 7.50 (dq, J = 7.2, 2.7, 2.2 Hz, 1H), 7.29 (dd, J = 10.5, 9.1 Hz, 1H), 4.17 (s, 2H), 3.58-3.44 (m, 7H), 2.01 (s, 4H). LCMS m/z = 413.3 (M + 1); RT = 1.26, Method 3. |
| 140 | 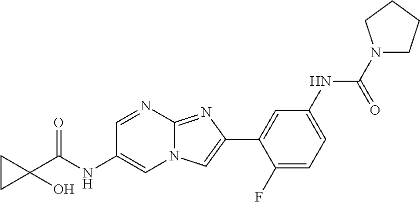 [SMILES: O=C(Nc1ccc(F)c(-c2cn3cc(NC(=O)C4(O)CC4)cnc3n2)c1)N1CCCC1] | ¹H NMR (400 MHz, CD₃OD) δ 9.81 (s, 1H), 9.10 (s, 1H), 8.40 (s, 1H), 8.03 (dd, J = 6.5, 2.6 Hz, 1H), 7.47 (ddd, J = 8.9, 4.4, 2.7 Hz, 1H), 7.29-7.18 (m, 1H), 3.49 (td, J = 7.0, 3.8 Hz, 4H), 2.00 (s, 4H), 1.38 (q, J = 4.6 Hz, 2H), 1.22- 1.04 (m, 2H). LCMS m/z = 425.2 (M + 1); RT = 1.28, Method 3. |
| 141 | 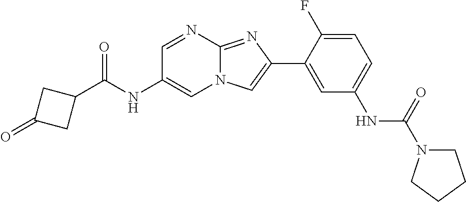 [SMILES: O=C1CC(C(=O)Nc2cnc3nc(-c4cc(NC(=O)N5CCCC5)ccc4F)cn3c2)C1] | ¹H NMR (400 MHz, CD₃OD: 9.80 (t, J = 2.9 Hz, 1H), 8.75 (d, J = 2.5 Hz, 1H), 8.39 (d, J = 2.9 Hz, 1H), 8.04 (dd, J = 6.6, 2.7 Hz, 1H), 7.61-7.45 (m, 1H), 7.34-7.14 (m, 1H), 3.56-3.37 (m, 9H), 2.00 (t, J = 6.5 Hz, 4H). LCMS m/z = 437.3 (M + 1); RT = 1.31, Method 3. |
| 142 | 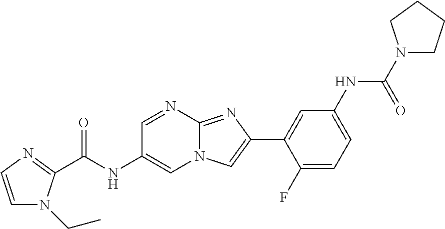 [SMILES: CCn1ccnc1C(=O)Nc1cnc2nc(-c3cc(NC(=O)N4CCCC4)ccc3F)cn2c1] | ¹H NMR (400 MHz, CD₃OD) δ 9.93 (d, J = 2.4 Hz, 1H), 9.07 (d, J = 2.5 Hz, 1H), 8.46 (d, J = 2.5 Hz, 1H), 8.06 (dd, J = 6.6, 2.7 Hz, 1H), 7.57-7.49 (m, 1H), 7.45 (s, 1H), 7.28 (dd, J = 10.5, 9.1 Hz, 1H), 7.19 (s, 1H), 4.59 (q, J = 7.2 Hz, 2H), 3.50 (d, J = 6.7 Hz, 4H), 2.01 (t, J = 6.4 Hz, 4H), 1.49 (t, J = 7.2 Hz, 3H). LCMS m/z = 463.3 (M + 1); RT = 1.44, Method 3. |
| 143 | 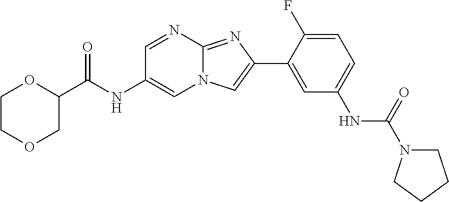 [SMILES: O=C(Nc1cnc2nc(-c3cc(NC(=O)N4CCCC4)ccc3F)cn2c1)C1COCCO1] | ¹H NMR (400 MHz, CD₃OD) δ 9.75 (d, J = 2.5 Hz, 1H), 8.96 (d, J = 2.5 Hz, 1H), 8.39 (d, J = 2.7 Hz, 1H), 8.03 (dd, J = 6.5, 2.7 Hz, 1H), 7.50 (ddd, J = 8.9, 4.5, 2.7 Hz, 1H), 7.24 (dd, J = 10.6, 8.9 Hz, 1H), 4.35 (dd, J = 9.7, 3.2 Hz, 1H), 4.10 (dd, J = 11.6, 3.1 Hz, 1H), 4.01 (dt, J = 11.9, 2.1 Hz, 1H), 3.86 (td, J = 11.7, 11.3, 2.8 Hz, 1H), 3.79 (dt, J = 11.9, 2.2 Hz, 1H), 3.72-3.57 (m, 2H), 3.55-3.43 (m, 4H), 2.05-1.94 (m, 4H). LCMS m/z = 455.3 (M + 1); RT = 1.33, Method 3. |
| 144 | 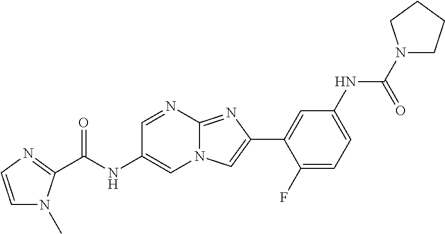 [SMILES: Cn1ccnc1C(=O)Nc1cnc2nc(-c3cc(NC(=O)N4CCCC4)ccc3F)cn2c1] | ¹H NMR (400 MHz, CD₃OD) δ 9.88 (d, J = 2.6 Hz, 1H), 9.01 (d, J = 2.5 Hz, 1H), 8.44 (d, J = 2.7 Hz, 1H), 8.05 (dd, J = 6.6, 2.7 Hz, 1H), 7.91 (s, 2H), 7.51 (ddd, J = 9.0, 4.5, 2.8 Hz, 1H), 7.26 (dd, J = 10.6, 8.9 Hz, 1H), 3.85 (s, 3H), 3.54-3.43 (m, 4H), 2.02-1.98 (m, 4H). LCMS m/z = 449 (M + 1); RT = 0.71, Method 2. |
| 145 | 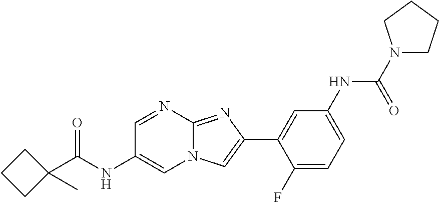 [SMILES: CC1(C(=O)Nc2cnc3nc(-c4cc(NC(=O)N5CCCC5)ccc4F)cn3c2)CCC1] | 1H NMR (400 MHz, CD₃OD) δ 9.73 (d, J = 2.5 Hz, 1H), 8.85 (d, J = 2.7 Hz, 1H), 8.37 (d, J = 2.8 Hz, 1H), 8.02 (dd, J = 6.5, 2.7 Hz, 1H), 7.51 (ddd, J = 8.9, 4.5, 2.8 Hz, 1H), 7.23 (dd, J = 10.7, 9.0 Hz, 1H), 3.55-3.44 (m, 4H), 2.58 (dtd, J = 10.2, 7.9, 1.9 Hz, 2H), 2.15-1.92 (m, 7H), 1.90-1.77 (m, 1H), 1.56 (s, 3H). LCMS m/z = 437.3 (M + 1); RT = 1.48, Method 3. |
| 146 | 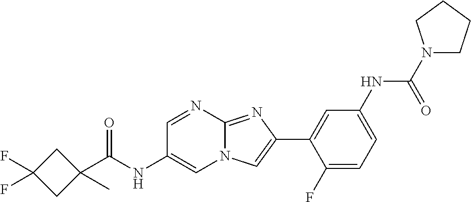 [SMILES: CC1(C(=O)Nc2cnc3nc(-c4cc(NC(=O)N5CCCC5)ccc4F)cn3c2)CC(F)(F)C1] | ¹H NMR (400 MHz, CD₃OD) δ 9.75 (d, J = 2.6 Hz, 1H), 8.82 (d, J = 2.6 Hz, 1H), 8.39 (d, J = 2.8 Hz, 1H), 8.03 (dd, J = 6.6, 2.7 Hz, 1H), 7.51 (ddd, J = 8.9, 4.5, 2.8 Hz, 1H), 7.24 (dd, J = 10.7, 8.9 Hz, 1H), 3.53-3.46 (m, 4H), 3.24-3.08 (m, 2H), 2.66-2.47 (m, 2H), 2.07-1.89 (m, 4H), 1.64 (s, 3H). LCMS m/z = 473 (M + 1); RT = 0.93, Method 2. |
| 147 | 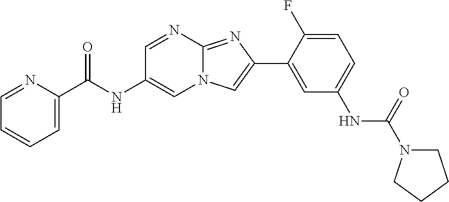 [SMILES: O=C(Nc1cnc2nc(-c3cc(NC(=O)N4CCCC4)ccc3F)cn2c1)c1ccccn1] | ¹H NMR (400 MHz, CD₃OD) δ 9.71 (d, J = 2.6 Hz, 1H), 8.86 (d, J = 2.6 Hz, 1H), 8.79-8.73 (m, 1H), 8.31-8.22 (m, 2H), 8.05 (td, J = 7.7, 1.7 Hz, 1H), 7.99 (dd, J = 6.7, 2.8 Hz, 1H), 7.65 (ddd, J = 7.7, 4.7, 1.1 Hz, 1H), 7.59 (ddd, J = 8.9, 4.5, 2.7 Hz, 1H), 7.16 (dd, J = 11.0, 8.9 Hz, 1H), 3.54-3.45 (m, 4H), 2.01-1.98 (m, 4H). LCMS m/z = 446 (M + 1); RT = 0.86, Method 2. |
| 148 | 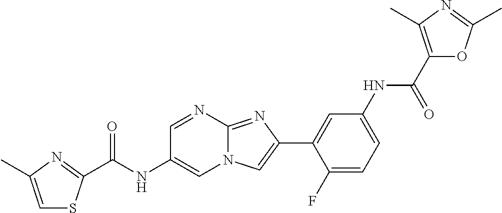 | ¹H NMR (400 MHz, DMSO-d₆) δ 11.27 (s, 1H), 10.33 (s, 1H), 9.69 (d, J = 2.6, 1H), 8.91 (d, J = 2.7, 1H), 8.72 (dd, J = 2.8, 6.9, 1H), 8.44 (d, J = 4.2, 1H), 7.82-7.78 (m, 1H), 7.77 (d, J = 0.9, 1H), 7.32 (dd, J = 9.0, 10.9, 1H), 2.54 (d, J = 0.8, 3H), 2.50 (s, 3H), 2.40 (s, 3H). LCMS m/z = 492 (M + 1); RT = 0.94, Method 2. |
| 149 | 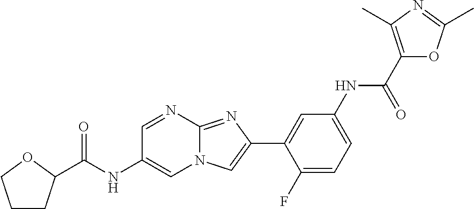 [SMILES: Cc1nc(C)c(C(=O)Nc2ccc(F)c(-c3cn4cc(NC(=O)C5CCCO5)cnc4n3)c2)o1] | ¹H NMR (400 MHz, DMSO-d₆) δ 10.36 (s, 1H), 10.27 (s, 1H), 9.66 (d, J = 2.5, 1H), 8.81 (d, J = 2.6, 1H), 8.66 (dd, J = 2.7, 6.9, 1H), 8.47 (d, J = 3.9, 1H), 7.77 (ddd, J = 2.8, 4.4, 8.9, 1H), 7.35 (dd, J = 9.0, 10.9, 1H), 4.50 (dd, J = 5.5, 8.3, 1H), 4.02 (dd, J = 6.7, 14.7, 1H), 3.89 (dd, J = 6.7, 14.8, 1H), 3.20-3.07 (m, 1H), 2.50 (s, 3H), 2.40 (s, 3H), 2.26 (dq, J = 7.5, 12.4, 1H), 2.03 (dt, J = 6.1, 18.8, 1H), 1.94-1.85 (m, 1H). LCMS m/z = 465.4 (M + 1); RT = 1.46, Method 3. |
| 150 | 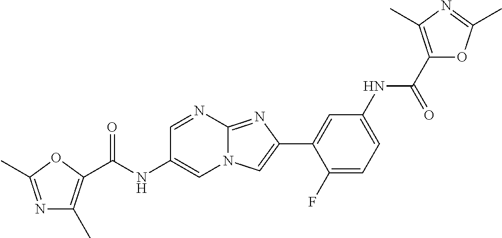 | ¹H NMR (400 MHz, DMSO-d₆) δ 10.67 (s, 1H), 10.34 (s, 1H), 9.65 (d, J = 2.6, 1H), 8.78 (d, J = 2.6, 1H), 8.71 (dd, J = 2.8, 6.9, 1H), 8.42 (d, J = 4.2, 1H), 7.79 (ddd, J = 2.8, 4.4, 8.8, 1H), 7.32 (dd, J = 9.0, 10.9, 1H), 2.53 (s, 5H), 2.41 (d, J = 7.5, 6H). LCMS m/z = 490.4 (M + 1); RT = 1.55, Method 3. |
| 151 | 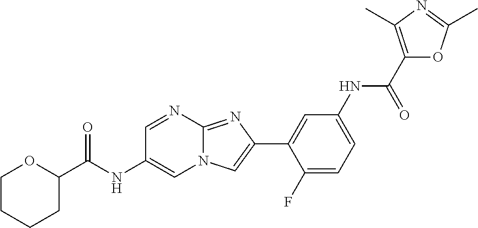 [SMILES: Cc1nc(C)c(C(=O)Nc2ccc(F)c(-c3cn4cc(NC(=O)C5CCCCO5)cnc4n3)c2)o1] | ¹H NMR (400 MHz, DMSO-d₆) δ 10.34 (s, 1H), 10.13 (s, 1H), 9.63 (d, J = 2.6, 1H), 8.77 (d, J = 2.6, 1H), 8.68 (dd, J = 2.7, 6.9, 1H), 8.42 (d, J = 4.1, 1H), 7.84-7.71 (m, 1H), 7.33 (dd, J = 9.0, 10.9, 1H), 4.15-3.97 (m, 2H), 2.50 (s, 3H), 2.40 (s, 3H), 2.04-1.82 (m, 2H), 1.67-1.38 (m, 4H). LCMS m/z = 479.2 (M + 1); RT = 2.96, Method 1. |
| 152 | 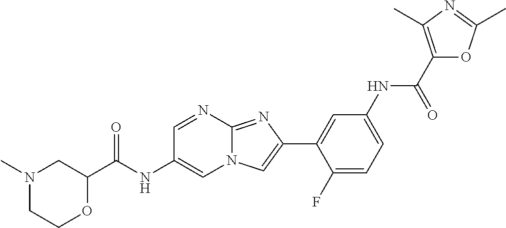 | ¹H NMR (400 MHz, DMSO-d₆) δ 10.56 (s, 1H), 10.33 (s, 1H), 9.56 (d, J = 2.6, 1H), 8.75-8.66 (m, 2H), 8.44 (d, J = 4.2, 1H), 7.78 (ddd, J = 2.8, 4.4, 8.9, 1H), 7.32 (dd, J = 9.0, 10.9, 1H), 4.73- 3.61 (m, 2H), 2.80-2.53 (m, 4H), 2.50 (s, 3H), 2.40 (s, 3H). LCMS m/z = 494.4 (M + 1); RT = 1.28, Method 3. |
| 153 | 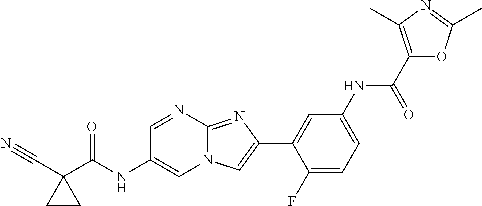 [SMILES: Cc1nc(C)c(C(=O)Nc2ccc(F)c(-c3cn4cc(NC(=O)C5(C#N)CC5)cnc4n3)c2)o1] | ¹H NMR (400 MHz, DMSO-d₆) δ 10.49 (s, 1H), 10.35 (s, 1H), 9.42 (d, J = 2.6, 1H), 8.72 (d, J = 2.6, 1H), 8.68 (dd, J = 2.8, 6.9, 1H), 8.43 (d, J = 4.0, 1H), 7.82-7.72 (m, 1H), 7.34 (dd, J = 9.0, 10.9, 1H), 2.50 (s, 3H), 2.40 (s, 3H), 1.83-1.68 (m, 4H). LCMS m/z = 460.3 (M + 1); RT = 1.52, Method 3. |
| 154 | 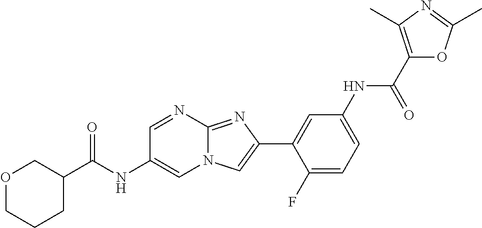 [SMILES: Cc1nc(C)c(C(=O)Nc2ccc(F)c(-c3cn4cc(NC(=O)C5CCCOC5)cnc4n3)c2)o1] | ¹H NMR (400 MHz, DMSO-d₆) δ 10.41 (s, 1H), 10.33 (s, 1H), 9.58 (d, J = 2.6, 1H), 8.69 (dd, J = 2.8, 6.9, 1H), 8.51 (d, J = 2.6, 1H), 8.39 (d, J = 4.2, 1H), 7.83-7.71 (m, 1H), 7.31 (dd, J = 9.0, 10.9, 1H), 4.00 (dd, J = 3.2, 11.7, 1H), 3.83 (d, J = 11.2, 1H), 3.44 (t, J = 10.7, 1H), 3.39-3.34 (m, 1H), 2.77-2.62 (m, 1H), 2.50 (s, 3H), 2.40 (s, 3H), 1.99 (dd, J = 2.6, 13.7, 1H), 1.81-1.48 (m, 3H). LCMS m/z = 479.4 (M + 1); RT = 1.48, Method 3. |
| 155 | 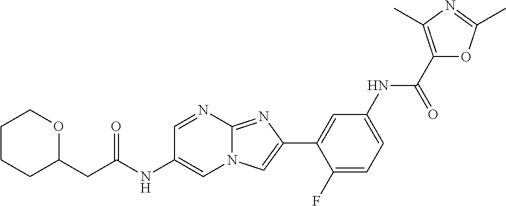 [SMILES: Cc1nc(C)c(C(=O)Nc2ccc(F)c(-c3cn4cc(NC(=O)CC5CCCCO5)cnc4n3)c2)o1] | ¹H NMR (400 MHz, DMSO-d₆) δ 10.40 (s, 1H), 10.33 (s, 1H), 9.62 (d, J = 2.6, 1H), 8.69 (dd, J = 2.8, 6.9, 1H), 8.50 (d, J = 2.7, 1H), 8.39 (d, J = 4.2, 1H), 7.78 (ddd, J = 2,8, 4.4, 8.9, 1H), 7.31 (dd, J = 8.9, 10.9, 1H), 3.90-3.81 (m, 1H), 3.79-3.68 (m, 1H), 3.37 (dtdd, J = 0.7, 1.4, 2.1, 5.5, 1H), 2.50 (s, 3H), 2.40 (s, 3H), 1.78 (d, J = 11.1, 1H), 1.66 (d, J = 12.8, 1H), 1.57-1.36 (m, 3H), 1.35-1.20 (m, 1H). LCMS m/z = 493.4 (M + 1); RT = 1.52, Method 3. |
| 156 | 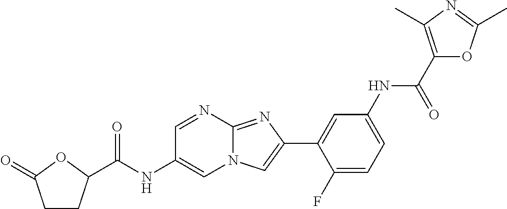 [SMILES: Cc1nc(C)c(C(=O)Nc2ccc(F)c(-c3cn4cc(NC(=O)C5CCC(=O)O5)cnc4n3)c2)o1] | ¹H NMR (400 MHz, DMSO-d₆) δ 10.74 (s, 1H), 10.34 (s, 1H), 9.58 (d, J = 2.7, 1H), 8.70 (dd, J = 2.8, 6.9, 1H), 8.63 (d, J = 2.7, 1H), 8.43 (d, J = 4.2, 1H), 7.78 (ddd, J = 2.8, 4.4, 8.9, 1H), 7.31 (dd, J = 9.0, 10.9, 1H), 5.20-5.11 (m, 1H), 2.63-2.51 (m, 2H), 2.50 (s, 3H), 2.40 (s, 3H), 1.26 (t, J = 7.2, 2H). LCMS m/z = 479.4 (M + 1); RT = 1.42, Method 3. |
| 157 | 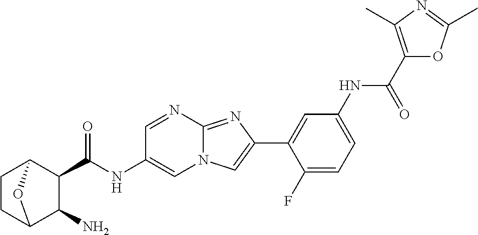 [SMILES: Cc1nc(C)c(C(=O)Nc2ccc(F)c(-c3cn4cc(NC(=O)[C@@H]5[C@@H]6CCC(O6)[C@@H]5N)cnc4n3)c2)o1] | ¹H NMR (400 MHz, DMSO-d₆) δ 11.08 (s, 1H), 10.35 (s, 1H), 9.50 (d, J = 2.6, 1H), 8.69 (dd, J = 2.7, 6.4, 2H), 8.50 (d, J = 4.0, 1H), 8.00 (d, J = 4.7, 3H), 7.77 (ddd, J = 2.8, 4.4, 8.8, 1H), 7.34 (dd, J = 9.0, 10.9, 1H), 4.81 (d, J = 2.9, 1H), 4.57 (d, J = 5.5, 1H), 3.80-3.73 (m, 2H), 3.17 (d, J = 8.4, 1H), 2.50 (s, 3H), 2.40 (s, 3H), 1.83-1.50 (m, 4H). LCMS m/z = 506.1 (M + 1); RT = 0.6, Method 2. |
| 158 | 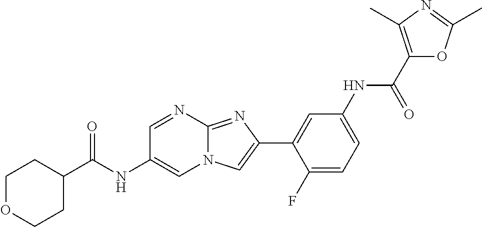 [SMILES: Cc1nc(C)c(C(=O)Nc2ccc(F)c(-c3cn4cc(NC(=O)C5CCOCC5)cnc4n3)c2)o1] | ¹H NMR (400 MHz, DMSO-d₆) δ 10.34 (d, J = 11.3, 2H), 9.61 (d, J = 2.7, 1H), 8.69 (dd, J = 2.8, 6.9, 1H), 8.53 (d, J = 2.6, 1H), 8.39 (d, J = 4.2, 1H), 7.77 (ddd, J = 2.8, 4.4, 8.9, 1H), 7.31 (dd, J = 8.9, 10.9, 1H), 3.98-3.88 (m, 2H), 3.43-3.35 (m, 2H), 2.76-2.61 (m, 1H), 2.50 (s, 3H), 2.40 (s, 3H), 1.78-1.64 (m, 4H). LCMS m/z = 479.4 (M + 1); RT = 1.44, Method 3. |
| 159 | 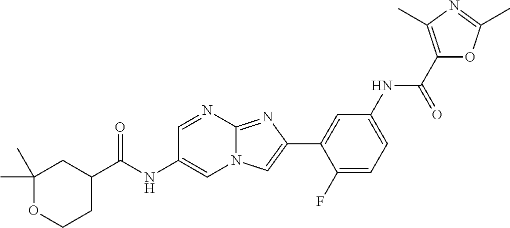 | ¹H NMR (400 MHz, DMSO-d₆) δ 10.34 (d, J = 10.0, 2H), 9.60 (d, J = 2.6, 1H), 8.69 (dd, J = 2.8, 6.9, 1H), 8.52 (d, J = 2.7, 1H), 8.39 (d, J = 4.2, 1H), 7.78 (ddd, J = 2.8, 4.4, 8.9, 1H), 7.31 (dd, J = 9.0, 10.9, 1H), 3.65 (ddd, J = 7.4, 11.4, 21.8, 2H), 2.92-2.75 (m, 1H), 2.50 (s, 3H), 2.40 (s, 3H), 1.76-1.41 (m, 4H), 1.22 (s, 3H), 1.17 (s, 3H). LCMS m/z = 507.4 (M + 1); RT = 1.55, Method 3. |
| 160 | 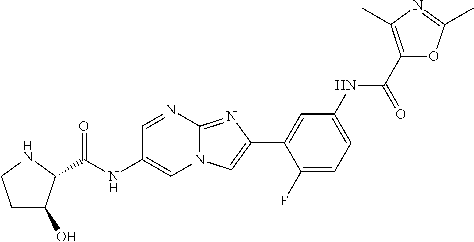 [SMILES: Cc1nc(C)c(C(=O)Nc2ccc(F)c(-c3cn4cc(NC(=O)[C@H]5NCC[C@@H]5O)cnc4n3)c2)o1] | ¹H NMR (400 MHz, DMSO-d₆) δ 11.66- 11.45 (m, 1H), 10.36 (s, 1H), 10.04 (s, 1H), 9.56 (s, 1H), 8.86 (dd, J = 4.6, 9.6, 1H), 8.71 (t, J = 8.4, 2H), 8.51 (t, J = 3.2, 1H), 7.84-7.70 (m, 1H), 7.34 (t, J = 9.9, 1H), 4.64-4.51 (m, 1H), 4.33 (s, 1H), 3.47-3.34 (m, 2H), 2.50 (s, 3H), 2.40 (s, 3H), 2.10-1.91 (m, 2H). LCMS m/z = 480.4 (M + 1); RT = 1.24, Method 3. |
| 161 | 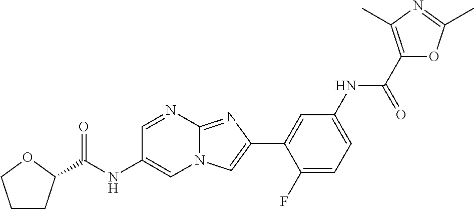 [SMILES: Cc1nc(C)c(C(=O)Nc2ccc(F)c(-c3cn4cc(NC(=O)[C@@H]5CCCO5)cnc4n3)c2)o1] | LCMS m/z = 465.4 (M + 1); RT = 1.46, Method 3. |
| 162 | 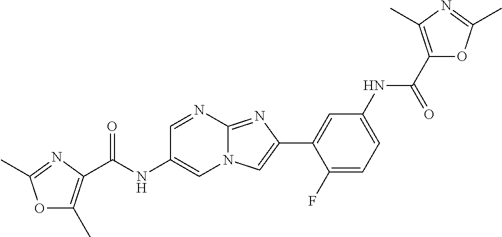 | ¹H NMR (400 MHz, DMSO-d₆) δ 10.59 (s, 1H), 10.33 (s, 1H), 9.71 (d, J = 2.6, 1H), 8.86 (d, J = 2.6, 1H), 8.71 (dd, J = 2.8, 6.9, 1H), 8.40 (d, J = 4.2, 1H), 7.78 (ddd, J = 2.8, 4.4, 8.9, 1H), 7.31 (dd, J = 9.0, 10.9, 1H), 2.62 (s, 3H), 2.50 (s, 3H), 2.39 (d, J = 7.1, 3H). LCMS m/z = 490.4 (M + 1); RT = 1.66, Method 3. |
| 163 | 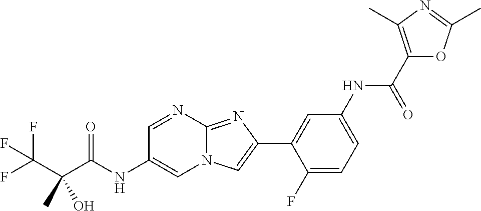 [SMILES: Cc1nc(C)c(C(=O)Nc2ccc(F)c(-c3cn4cc(NC(=O)[C@](C)(O)C(F)(F)F)cnc4n3)c2)o1] | ¹H NMR (400 MHz, DMSO-d₆) δ 10.33 (s, 1H), 9.63 (d, J = 2.6, 1H), 8.80 (d, J = 2.7, 1H), 8.70 (dd, J = 2.8, 6.9, 1H), 8.39 (d, J = 4.2, 1H), 7.78 (ddd, J = 2.8, 4.4, 8.9, 1H), 7.31 (dd, J = 8.9, 10.9, 1H), 2.50 (s, 3H), 2.40 (s, 3H), 1.62 (s, 3H). LCMS m/z = 507.2 (M + 1); RT = 1.55, Method 3. |
| 164 | 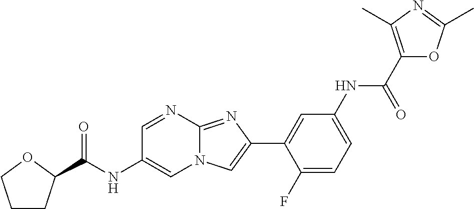 [SMILES: Cc1nc(C)c(C(=O)Nc2ccc(F)c(-c3cn4cc(NC(=O)[C@H]5CCCO5)cnc4n3)c2)o1] | LCMS m/z = 465.4 (M + 1); RT = 1.44, Method 3. |
| 165 | 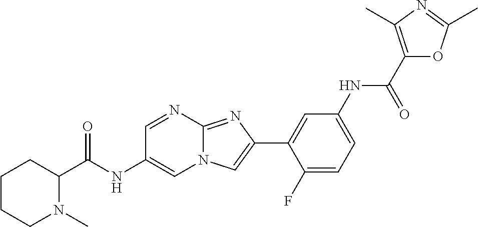 [SMILES: Cc1nc(C)c(C(=O)Nc2ccc(F)c(-c3cn4cc(NC(=O)C5CCCCN5C)cnc4n3)c2)o1] | ¹H NMR (400 MHz, DMSO-d₆) δ 11.38 (s, 1H), 10.34 (s, 1H), 9.95 (s, 1H), 9.57 (d, J = 2.6, 1H), 8.73 (dd, J = 2.8, 6.9, 1H), 8.63 (d, J = 2.6, 1H), 8.47 (d, J = 4.1, 1H), 7.95 (s, 1H), 7.84-7.70 (m, 1H), 7.33 (dd, J = 9.0, 11.0, 1H), 4.00 (t, J = 12.2, 1H), 2.83 (d, J = 4.6, 3H), 2.74-2.70 (m, 4H), 2.50 (s, 3H), 2.40 (s, 3H), 2.36-2.22 (m, 1H), 1.85 (d, J = 12.4, 2H), 1.70 (dd, J = 14.6, 28.5, 2H), 1.51 (t, J = 9.5, 1H). LCMS m/z = 492.5 (M + 1); RT = 1.31, Method 3. |
| 166 | 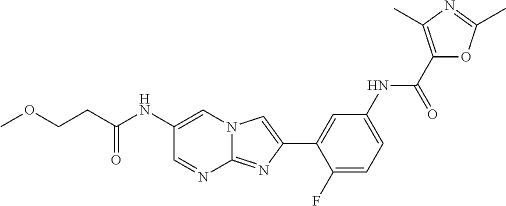 | ¹H NMR (400 MHz, DMSO-d₆) δ 10.44 (s, 1H), 10.33 (s, 1H), 9.61 (d, J = 2.6, 1H), 8.69 (dd, J = 2.8, 6.9, 1H), 8.51 (d, J = 2.6, 1H), 8.40 (d, J = 4.2, 1H), 7.78 (ddd, J = 2.8, 4.4, 8.9, 1H), 7.31 (dd, J = 8.9, 10.9, 1H), 3.66 (t, J = 6.1, 2H), 3.27-3.25 (m, 3H), 2.91-2.86 (m, 1H), 2.75-2.71 (m, 1H), 2.63 (t, J = 6.1, 2H), 2.50 (s, 3H), 2.40 (s, 3H). LCMS m/z = 453.4 (M + 1); RT = 1.42, Method 3. |
| 167 | 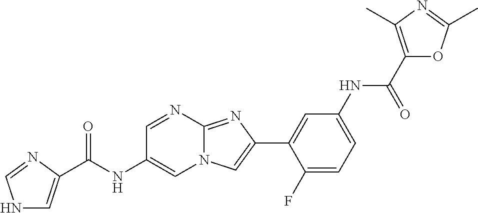 [SMILES: Cc1nc(C)c(C(=O)Nc2ccc(F)c(-c3cn4cc(NC(=O)c5c[nH]cn5)cnc4n3)c2)o1] | LCMS m/z = 461.2 (M + 1); RT = 1.26, Method 3. |
| 168 | 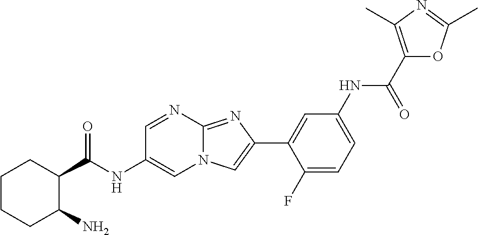 [SMILES: Cc1nc(C)c(C(=O)Nc2ccc(F)c(-c3cn4cc(NC(=O)[C@@H]5CCCC[C@@H]5N)cnc4n3)c2)o1] | LCMS m/z = 492.1 (M + 1); RT = 0.64, Method 2. |
| 169 | 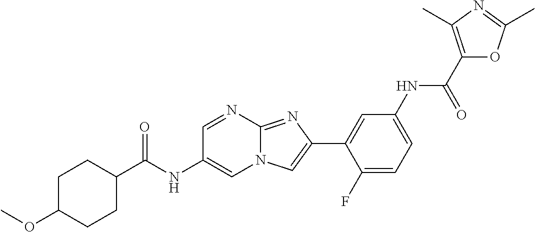 | LCMS m/z = 507.4 (M + 1); RT = 1.54, Method 3. |
| 170 | 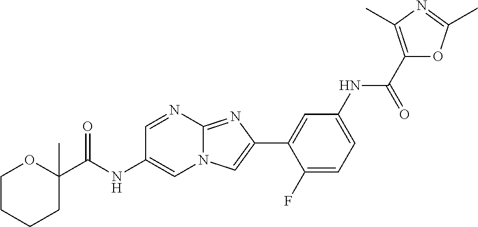 [SMILES: Cc1nc(C)c(C(=O)Nc2ccc(F)c(-c3cn4cc(NC(=O)C5(C)CCCCO5)cnc4n3)c2)o1] | ¹H NMR (400 MHz, DMSO-d₆) δ 10.32 (d, J = 15.1, 2H), 9.65 (d, J = 2.6, 1H), 8.81 (d, J = 2.6, 1H), 8.69 (dd, J = 2.8, 6.9, 1H), 8.40 (d, J = 4.1, 1H), 7.77 (ddd, J = 2.8, 4.4, 8.9, 1H), 7.32 (dd, J = 9.0, 10.9, 1H), 3.86 (d, J = 12.0, 1H), 3.56 (s, 2H), 2.50 (s, 3H), 2.40 (s, 3H), 2.22-2.12 (m, 1H), 1.67 (s, 1H), 1.46 (d, J = 26.8, 4H), 1.39 (s, 3H). LCMS m/z = 493.4 (M + 1); RT = 1.63, Method 3. |
| 171 | 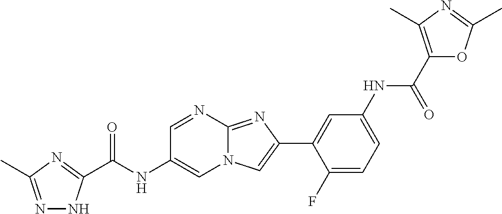 | ¹H NMR (400 MHz, DMSO-d₆) δ 10.92 (s, 1H), 10.33 (s, 1H), 9.71 (t, J = 4.8, 1H), 8.88 (t, J = 4.1, 1H), 8.71 (dd, J = 2.7, 6.9, 1H), 8.44 (d, J = 4.2, 1H), 7.78 (ddd, J = 2.9, 4.4, 8.8, 1H), 7.32 (dd, J = 9.0, 10.9, 1H), 2.51 (s, 3H), 2.48 (s, 3H), 2.40 (s, 3H). LCMS m/z = 476 (M + 1); RT = 0.72, Method 2. |
| 172 | 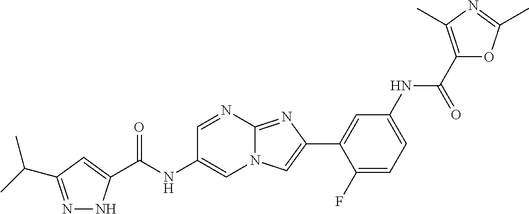 [SMILES: Cc1nc(C)c(C(=O)Nc2ccc(F)c(-c3cn4cc(NC(=O)c5cc(C(C)C)n[nH]5)cnc4n3)c2)o1] | MS m/z = 504 (M + 1). |
| 173 | 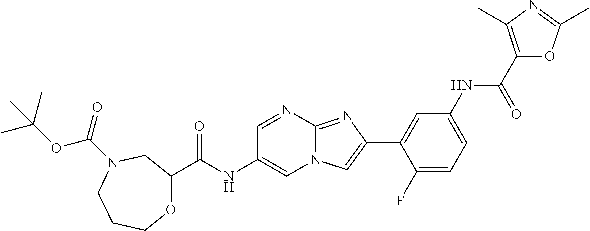 [SMILES: Cc1nc(C)c(C(=O)Nc2ccc(F)c(-c3cn4cc(NC(=O)C5CN(C(=O)OC(C)(C)C)CCCO5)cnc4n3)c2)o1] | ¹H NMR (400 MHz, DMSO-d₆) δ 10.34 (s, 1H), 10.22 (d, J = 6.0, 1H), 9.63 (d, J = 28.7, 1H), 8.76 (d, J = 24.8, 1H), 8.68 (dd, J = 2.7, 6.9, 1H), 8.43 (s, 1H), 7.84-7.72 (m, 1H), 7.32 (dd, J = 9.0, 10.9, 1H), 4.31 (s, 1H), 4.15 (s, 1H), 4.06-3.87 (m, 1H), 3.66 (s, 2H), 2.50 (s, 3H), 2.40 (s, 3H), 1.87 (s, 2H), 1.37 (d, J = 29.2, 9H). LCMS m/z = 594.4 (M + 1); RT = 1.72, Method 3. |
| 174 | 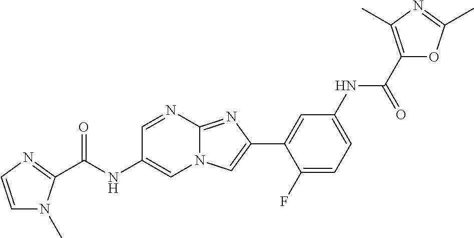 [SMILES: Cc1nc(C)c(C(=O)Nc2ccc(F)c(-c3cn4cc(NC(=O)c5nccn5C)cnc4n3)c2)o1] | ¹H NMR (400 MHz, DMSO-d₆) δ 10.94 (s, 1H), 10.33 (s, 1H), 9.71 (d, J = 2.7, 1H), 8.90 (d, J = 2.7, 1H), 8.71 (td, J = 2.7, 7.0, 1H), 8.42 (t, J = 7.0, 1H), 7.95 (s, 1H), 7.78 (ddd, J = 2.8, 4.4, 8.9, 1H), 7.51 (s, 1H), 7.32 (dd, J = 8.9, 10.9, 1H), 7.15 (d, J = 0.9, 1H), 2.56- 2.53 (m, 4H), 2.50 (s, 3H), 2.40 (s, 3H). LCMS m/z = 475.1 (M + 1); RT = 0.83, Method 2 . |
| 175 | 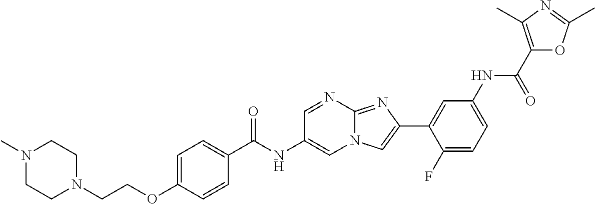 | ¹H NMR (400 MHz, DMSO-d₆) δ 10.52 (s, 1H), 10.34 (s, 1H), 9.69 (d, J = 2.6, 1H), 8.78 (d, J = 2.7, 1H), 8.71 (dd, J = 2.8, 6.9, 1H), 8.44 (d, J = 4.1, 1H), 8.03 (d, J = 8.9, 2H), 7.83-7.69 (m, 1H), 7.33 (dd, J = 9.0, 10.9, 1H), 7.13 (d, J = 8.9, 2H), 4.28-4.15 (m, 2H), 3.04-2.67 (m, 8H), 2.50 (s, 3H), 2.40 (s, 3H). LCMS m/z = 613.5 (M + 1); RT = 1.39, Method 3. |
| 176 | 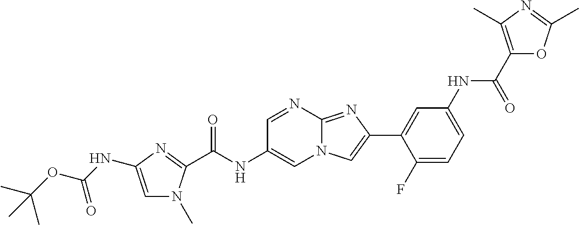 [SMILES: Cc1nc(C)c(C(=O)Nc2ccc(F)c(-c3cn4cc(NC(=O)c5nc(NC(=O)OC(C)(C)C)cn5C)cnc4n3)c2)o1] | LCMS m/z = 590.4 (M + 1); RT = 1.78, Method 3. |
| 177 | 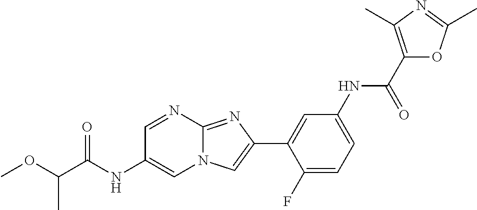 | ¹H NMR (400 MHz, DMSO-d₆) δ 10.35 (d, J = 6.1, 2H), 9.64 (d, J = 2.6, 1H), 8.77-8.65 (m, 2H), 8.43 (d, J = 4.1, 1H), 7.77 (ddd, J = 2.8, 4.4, 8.9, 1H), 7.32 (dd, J = 9.0, 10.9, 1H), 3.97 (q, J = 6.7, 1H), 3.40-3.32 (m, 3H), 2.50 (s, 3H), 2.40 (s, 3H), 1.37 (d, J = 6.7, 3H). LCMS m/z = 453.3 (M + 1); RT = 1.46, Method 3. |
| 178 | 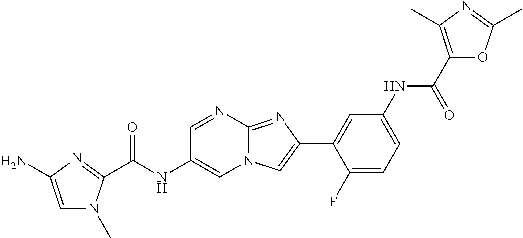 [SMILES: Cc1nc(C)c(C(=O)Nc2ccc(F)c(-c3cn4cc(NC(=O)c5nc(N)cn5C)cnc4n3)c2)o1] | LCMS m/z = 490.1 (M + 1); RT = 0.74, Method 2. |
| 179 | 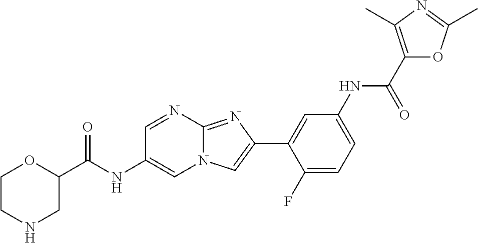 [SMILES: Cc1nc(C)c(C(=O)Nc2ccc(F)c(-c3cn4cc(NC(=O)C5CNCCO5)cnc4n3)c2)o1] | ¹H NMR (400 MHz, DMSO-d₆) δ 10.72 (s, 1H), 10.37 (s, 1H), 9.71 (s, 1H), 9.63 (d, J = 2.5, 1H), 9.48 (s, 1H), 8.82 (d, J = 2.5, 1H), 8.67 (d, J = 6.8, 1H), 8.51 (d, J = 3.8, 1H), 7.77 (dd, J = 5.8, 10.2, 1H), 7.44-7.24 (m, 1H), 4.62 (d, J = 10.6, 1H), 4.00 (dd, J = 10.7, 17.9, 2H), 3.53 (d, J = 12.4, 1H), 3.27 (d, J = 12.4, 1H), 3.15-3.00 (m, 1H), 2.50 (s, 3H), 2.40 (s, 3H). LCMS m/z = 480.4 (M + 1); RT = 1.26, Method 3. |
| 180 | 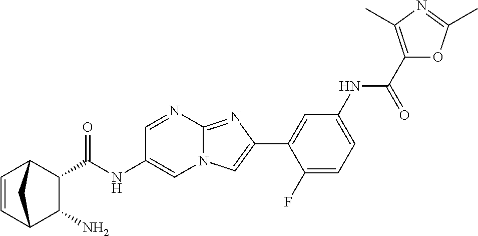 [SMILES: Cc1nc(C)c(C(=O)Nc2ccc(F)c(-c3cn4cc(NC(=O)[C@@H]5[C@H](N)[C@@H]6C=C[C@H]5C6)cnc4n3)c2)o1] | ¹H NMR (400 MHz, DMSO-d₆) δ 11.02 (s, 1H), 10.35 (s, 1H), 9.42 (d, J = 2.6, 1H), 8.69 (dt, J = 2.5, 4.7, 2H), 8.44 (d, J = 4.0, 1H), 7.85-7.62 (m, 4H), 7.33 (dd, J = 8.9, 10.9, 1H), 6.36 (dd, J = 2.9, 5.6, 1H), 6.24 (dd, J = 2.9, 5.6, 1H), 4.09-3.90 (m, 1H), 3.38 (dd, J = 3.2, 8.8, 1H), 3.31 (s, 1H), 3.19 (s, 1H), 2.50 (s, 3H), 2.40 (s, 3H), 1.55 (d, J = 8.8, 1H), 1.47 (d, J = 8.9, 1H). LCMS m/z = 502.1 (M + 1); RT = 0.64, Method 2. |
| 181 | 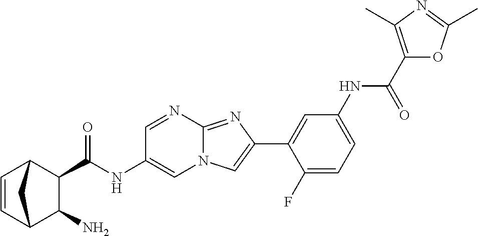 [SMILES: Cc1nc(C)c(C(=O)Nc2ccc(F)c(-c3cn4cc(NC(=O)[C@H]5[C@@H](N)[C@@H]6C=C[C@H]5C6)cnc4n3)c2)o1] | ¹H NMR (400 MHz, DMSO-d₆) δ 10.87 (s, 1H), 10.34 (s, 1H), 9.47 (d, J = 2.6, 1H), 8.71 (dd, J = 2.8, 6.9, 1H), 8.62 (d, J = 2.6, 1H), 8.46 (t, J = 6.2, 1H), 8.01 (s, 3H), 7.95 (s, 1H), 7.81-7.72 (m, 1H), 7.32 (dd, J = 9.0, 10.9, 1H), 6.42 (dd, J = 2.9, 5.6, 1H), 6.30 (dd, J = 3.1, 5.6, 1H), 3.09 (s, 1H), 2.95 (s, 1H), 2.89 (s, 2H), 2.75-2.69 (m, 3H), 2.50 (s, 3H), 2.40 (s, 3H). LCMS m/z = 502.1 (M + 1); RT = 0.65, Method 2. |
| 182 | 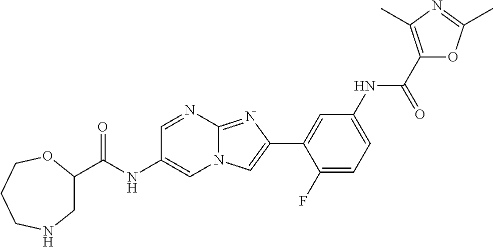 [SMILES: Cc1nc(C)c(C(=O)Nc2ccc(F)c(-c3cn4cc(NC(=O)C5CNCCCO5)cnc4n3)c2)o1] | LCMS m/z = 494.4 (M + 1); RT = 1.26, Method 3. |
| 183 | 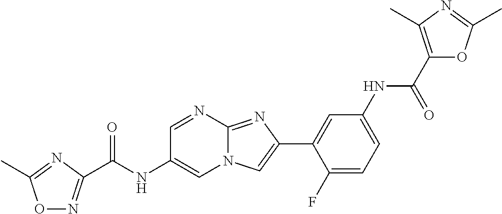 | ¹H NMR (400 MHz, DMSO-d₆) δ 10.54 (s, 1H), 10.35 (s, 1H), 9.58 (s, 1H), 9.21 (s, 2H), 8.77 (s, 1H), 8.69 (dd, J = 2.7, 6.9, 1H), 8.46 (d, J = 3.8, 1H), 7.77 (dd, J = 3.6, 8.1, 1H), 7.40-7.22 (m, 1H), 4.69 (d, J = 9.7, 1H), 4.15- 3.98 (m, 1H), 4.00-3.85 (m, 1H), 3.75- 3.64 (m, 2H), 3.22 (s, 1H), 2.50 (s, 3H), 2.40 (s, 3H), 2.08 (s, 2H). LCMS m/z = 477 (M + 1); RT = 0.81, Method 2. |
| 184 and 185 | 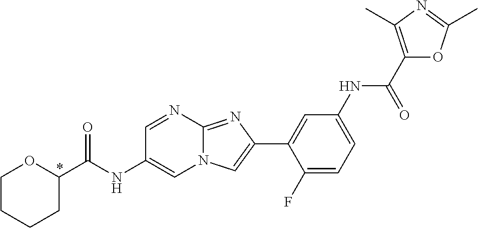 [SMILES: Cc1nc(C)c(C(=O)Nc2ccc(F)c(-c3cn4cc(NC(=O)C5CCCCO5)cnc4n3)c2)o1] | Enantiomer 1: LCMS m/z = 479 (M + 1); RT = 2.60, Method 5. Enantiomer 2: LCMS m/z = 479 (M + 1); RT = 3.95, Method 5. |
| 186 | 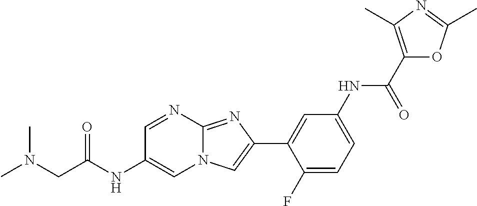 | LCMS m/z = 452.4 (M + 1); RT = 1.24, Method 3. |
| 187 | 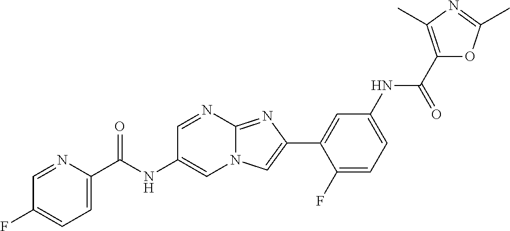 [SMILES: Cc1nc(C)c(C(=O)Nc2ccc(F)c(-c3cn4cc(NC(=O)c5ccc(F)cn5)cnc4n3)c2)o1] | LCMS m/z = 490 (M + 1); RT = 0.92, Method 2. |
| 188 | 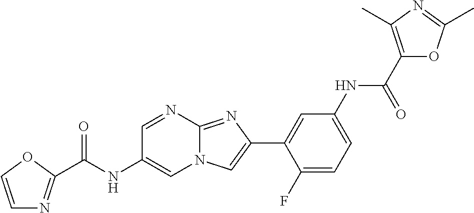 [SMILES: Cc1nc(C)c(C(=O)Nc2ccc(F)c(-c3cn4cc(NC(=O)c5ncco5)cnc4n3)c2)o1] | LCMS m/z = 462.4 (M + 1); RT = 1.46, Method 3. |
| 189 | 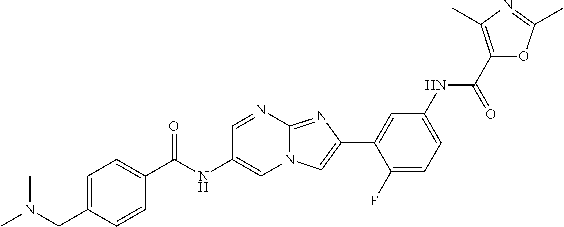 | LCMS m/z = 528.4 (M + 1); RT = 1.39, Method 3. |
| 190 | 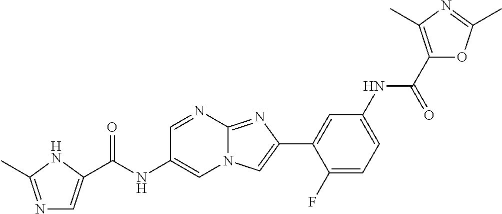 | LCMS m/z = 475.4 (M + 1); RT = 1.29, Method 3. |
| 191 | 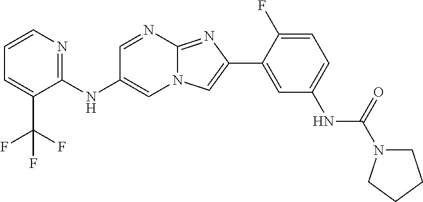 [SMILES: O=C(Nc1ccc(F)c(-c2cn3cc(Nc4ncccc4C(F)(F)F)cnc3n2)c1)N1CCCC1] | LCMS m/z = 486 (M + 1); RT = 0.95, Method 2. |
| 192 | 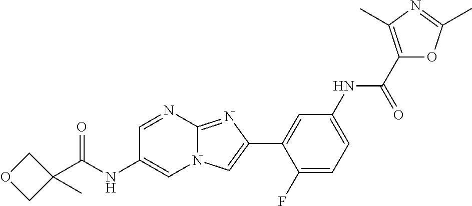 [SMILES: Cc1nc(C)c(C(=O)Nc2ccc(F)c(-c3cn4cc(NC(=O)C5(C)COC5)cnc4n3)c2)o1] | LCMS m/z = 465.4 (M + 1); RT = 1.42, Method 3. |
| 193 | 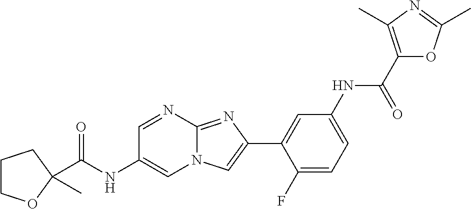 [SMILES: Cc1nc(C)c(C(=O)Nc2ccc(F)c(-c3cn4cc(NC(=O)C5(C)CCCO5)cnc4n3)c2)o1] | LCMS m/z = 479.4 (M + 1); RT = 1.54, Method 3. |
| 194 | 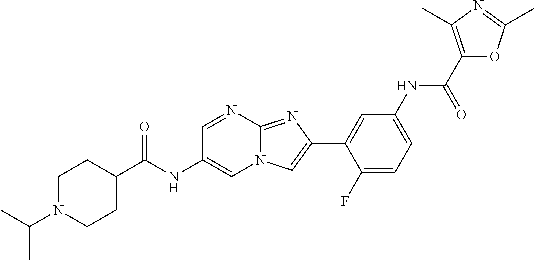 [SMILES: Cc1nc(C)c(C(=O)Nc2ccc(F)c(-c3cn4cc(NC(=O)C5CCN(C(C)C)CC5)cnc4n3)c2)o1] | LCMS m/z = 520.5 (M + 1); RT = 1.31, Method 3. |
| 195 | 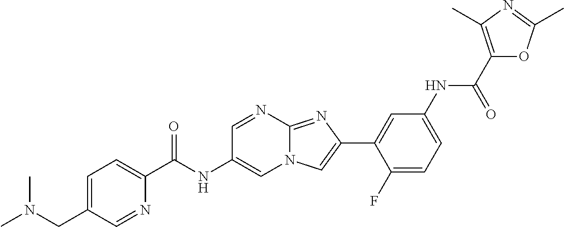 | LCMS m/z = 529.4 (M + 1); RT = 1.37, Method 3. |
| 196 | 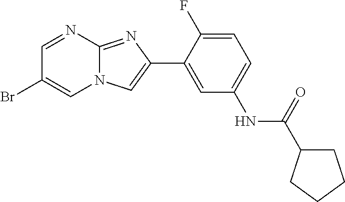 [SMILES: O=C(Nc1ccc(F)c(-c2cn3cc(Br)cnc3n2)c1)C1CCCC1] | ¹H NMR (400 MHz, DMSO-d₆) δ 10.05 (s, 1H), 9.35 (d, J = 2.5 Hz, 1H), 8.64 (d, J = 2.5 Hz, 1H), 8.51 (dd, J = 6.8, 2.8 Hz, 1H), 8.23 (d, J = 4.1 Hz, 1H), 7.73 (ddd, J = 8.9, 4.6, 2.8 Hz, 1H), 7.28 (dd, J = 11.1, 8.9 Hz, 1H), 2.79 (p, J = 8.0 Hz, 1H), 1.93-1.80 (m, 2H), 1.81-1.53 (m, 6H). LCMS m/z = 403.2 (M + 1), 405.2 (M + 3). RT = 1.66, Method 3. |
| 197 | 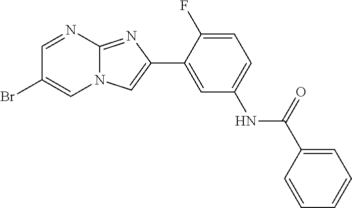 [SMILES: O=C(Nc1ccc(F)c(-c2cn3cc(Br)cnc3n2)c1)c1ccccc1] | LCMS m/z = 411.2 (M + 1), 413.2 (M + 3). RT = 1.66, Method 3. |
| 198 | 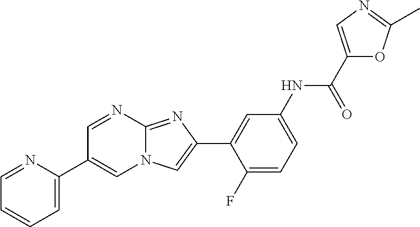 [SMILES: Cc1ncc(C(=O)Nc2ccc(F)c(-c3cn4cc(-c5ccccn5)cnc4n3)c2)o1] | ¹H NMR (400 MHz, DMSO-d₆) δ 10.51 (s, 1H), 9.71 (d, J = 2.51 Hz, 1H), 9.29 (d, J = 2.26 Hz, 1H), 8.73 (d, J = 4.27 Hz, 1H), 8.64 (dd, J = 6.78, 2.76 Hz, 1H), 8.41 (d, J = 4.02 Hz, 1H), 8.08 (d, J = 8.03 Hz, 1H), 7.98 (td, J = 7.72, 1.63 Hz, 1H), 7.90 (s, 1H), 7.83-7.89 (m, 1H), 7.45 (dd, J = 7.15, 5.14 Hz, 1H), 7.37 (dd, J = 11.04, 9.03 Hz, 1H), 2.54 (s, 3H). LCMS m/z = 415.2 (M + 1); RT = 1.66, Method 3. |
| 199 | 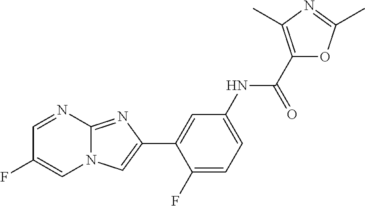 [SMILES: Cc1nc(C)c(C(=O)Nc2ccc(F)c(-c3cn4cc(F)cnc4n3)c2)o1] | ¹H NMR (600 MHz, CD₃OD) δ 9.15 (dd, J = 3.7, 2.8 Hz, 1H), 8.84 (d, J = 2.8 Hz, 1H), 8.37 (d, J = 3.2 Hz, 1H), 8.33 (dd, J = 6.6, 2.7 Hz, 1H), 7.81 (ddd, J = 8.9, 4.4, 2.7 Hz, 1H), 7.31 (dd, J = 10.8, 8.9 Hz, 1H), 2.56 (s, 3H), 2.47 (s, 3H). LCMS m/z = 370.3 (M + 1); RT = 1.5, Method 3. |
| 200 | 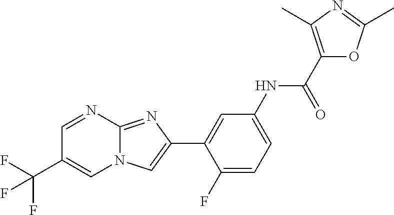 [SMILES: Cc1nc(C)c(C(=O)Nc2ccc(F)c(-c3cn4cc(C(F)(F)F)cnc4n3)c2)o1] | ¹H NMR (400 MHz, DMSO-d₆) δ 10.36 (s, 1H), 9.69 (s, 1H), 8.92 (d, J = 4.0 Hz, 1H), 8.76-8.78 (m, 1H), 8.41 (d, J = 4.0 Hz, 1H), 7.82-7.86 (m, 1H), 7.34- 7.39 (m, 1H) 2.51 (s, 3H), 2.40 (s, 3H). LCMS m/z = 420 (M + 1); RT = 0.94, Method 2. |
| 201 | 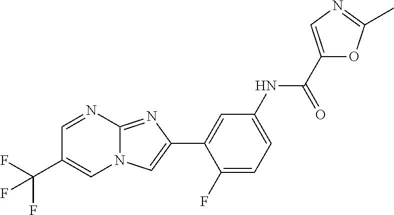 [SMILES: Cc1ncc(C(=O)Nc2ccc(F)c(-c3cn4cc(C(F)(F)F)cnc4n3)c2)o1] | ¹H NMR (400 MHz, DMSO-d₆) δ 10.52 (s, 1H), 9.68 (s, 1H), 8.92 (d, J = 4.0 Hz, 1H), 8.65-8.68 (m, 1H), 8.42 (d, J = 4.0 Hz, 1H), 7.9 (d, J = 4.0 Hz, 2H), 8.39 (t, J = 8.0 Hz, 1H), 2.51 (s, 3H). LCMS m/z = 406 (M + 1); RT = 0.88, Method 2. |
| 202 | 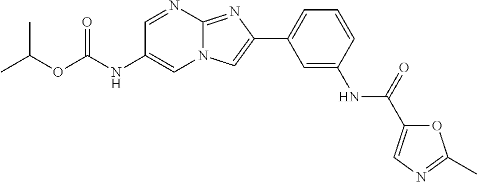 [SMILES: Cc1ncc(C(=O)Nc2cccc(-c3cn4cc(NC(=O)OC(C)C)cnc4n3)c2)o1] | LCMS m/z = 421.3 (M + 1); RT = 1.41, Method 3. |
| 203 | 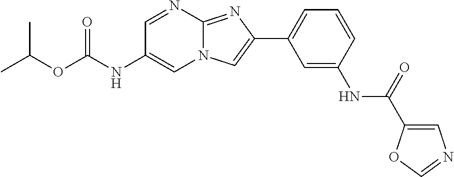 [SMILES: CC(C)OC(=O)Nc1cnc2nc(-c3cccc(NC(=O)c4cnco4)c3)cn2c1] | ¹H NMR (400 MHz, CD₃OD) δ 9.48 (s, 1H), 8.66 (d, J = 2.6 Hz, 1H), 8.44 (s, 1H), 8.36 (s, 1H), 8.29 (t, J = 2.0 Hz, 1H), 7.93 (s, 1H), 7.74 (dt, J = 8.2, 1.4 Hz, 1H), 7.69 (dt, J = 7.9, 1.4 Hz, 1H), 7.53 (t, J = 7.9 Hz, 1H), 5.04 (hept, J = 6.4 Hz, 1H), 1.34 (d, J = 6.2 Hz, 6H). LCMS m/z = 407 (M + 1); RT = 0.75, Method 2. |
| 204 | 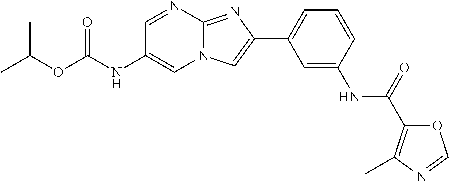 [SMILES: Cc1ncoc1C(=O)Nc1cccc(-c2cn3cc(NC(=O)OC(C)C)cnc3n2)c1] | LCMS m/z = 421.3 (M + 1); RT = 1.44, Method 3. |
| 205 | 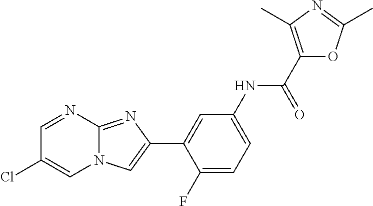 [SMILES: Cc1nc(C)c(C(=O)Nc2ccc(F)c(-c3cn4cc(Cl)cnc4n3)c2)o1] | ¹H NMR (600 MHz, CD₃OD) δ 9.28- 9.21 (m, 1H), 8.82-8.75 (m, 1H), 8.40- 8.33 (m, 2H), 7.80 (ddd, J = 9.0, 4.5, 2.7 Hz, 1H), 7.38-7.28 (m, 1H), 2.56 (s, 3H), 2.47 (s, 3H). LCMS m/z = 386 (M + 1); RT = 0.87, Method 2. |
| 206 | 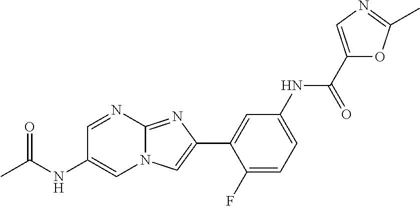 [SMILES: CC(=O)Nc1cnc2nc(-c3cc(NC(=O)c4cnc(C)o4)ccc3F)cn2c1] | ¹H NMR (400 MHz, DMSO-d₆) δ 10.49 (s, 1H), 10.41 (s, 1H), 9.57 (d, J = 2.5, 1H), 8.62-8.50 (m, 1H), 8.50 (d, J = 2.6, 1H), 8.42 (d, J = 4.2, 1H), 7.90 (s, 1H), 7.83 (s, 1H), 7.39-7.26 (m, 1H), 2.54 (s, 3H), 2.13 (s, 3H). LCMS m/z = 395.4 (M + 1); RT = 1.29, Method 3. |
| 207 | 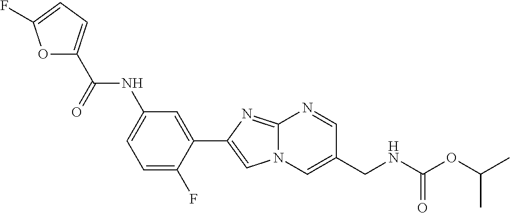 [SMILES: CC(C)OC(=O)NCc1cnc2nc(-c3cc(NC(=O)c4ccc(F)o4)ccc3F)cn2c1] | LCMS m/z = 455.9 (M + 1); RT = 1.03, Method 2. |
| 208 | 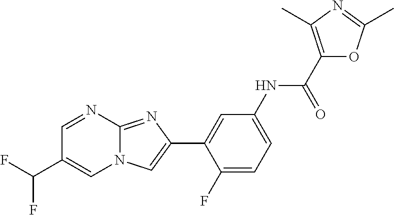 [SMILES: Cc1nc(C)c(C(=O)Nc2ccc(F)c(-c3cn4cc(C(F)F)cnc4n3)c2)o1] | ¹H NMR (400 MHz, DMSO-d₆) δ 10.35 (s, 1H), 9.37 (s, 1H), 8.69-8.84 (s, 2H), 8.43 (s, 1H), 7.83 (ddd, J = 8.85, 4.33, 2.89 Hz, 1H), 7.10-7.47 (m, 2H), 2.51 (s, 3H), 2.41 (s, 3H). LCMS m/z = 402.2 (M + 1); RT = 2.86, Method 1. |
| 209 | 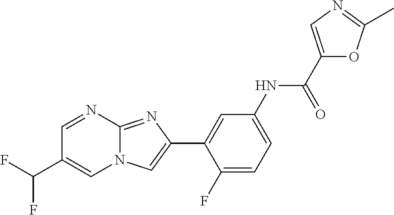 [SMILES: Cc1ncc(C(=O)Nc2ccc(F)c(-c3cn4cc(C(F)F)cnc4n3)c2)o1] | ¹H NMR (400 MHz, DMSO-d₆) δ 10.35 (s, 1H), 9.37 (s, 1H), 8.69-8.84 (s, 2H), 8.43 (s, 1H), 7.83 (ddd, J = 8.85, 4.33, 2.89 Hz, 1H), 7.10-7.47 (m, 2H), 2.51 (s, 3H), 2.41 (s, 3H). LCMS m/z = 388 (M + 1); RT = 0.79, Method 2. |
| 210 | 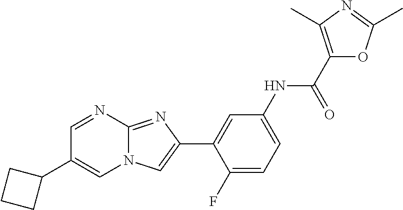 [SMILES: Cc1nc(C)c(C(=O)Nc2ccc(F)c(-c3cn4cc(C5CCC5)cnc4n3)c2)o1] | ¹H NMR (400 MHz, DMSO-d₆): 10.32 (s, 1H), 8.90 (d, J = 1.51 Hz, 1H), 8.72 (dd, J = 7.03, 2.76 Hz, 1H), 8.56 (d, J = 2.26 Hz, 1H), 8.23 (d, J = 4.27 Hz, 1H), 7.72-7.84 (m, 1H), 7.31 (dd, J = 11.04, 9.03 Hz, 1H), 3.61-3.65 (m, 1H), 2.51 (s, 3H), 2.41 (s, 3H), 2.32-2.39 (m, 2H), 2.14-2.25 (m, 2H), 2.01-2.09 (m, 1H), 1.88 (m, 1H). LCMS m/z = 406.1 (M + 1); RT = 0.94, Method 2. |
| 211 | 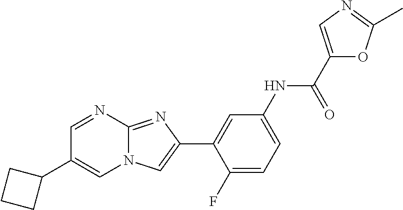 [SMILES: Cc1ncc(C(=O)Nc2ccc(F)c(-c3cn4cc(C5CCC5)cnc4n3)c2)o1] | LCMS m/z = 392.1 (M + 1); RT = 0.9, Method 2. |
| 212 | 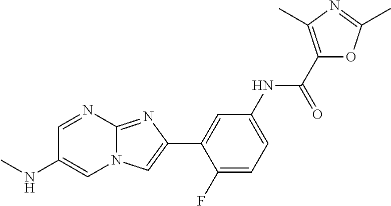 | ¹H NMR: (400 MHz, DMSO-d₆) δ 10.27 (brs, 1H), 8.63 (dd, J = 6.78, 2.51 Hz, 1H), 8.26 (d, J = 2.76 Hz, 1H), 8.08 (d, J = 4.27 Hz, 1H), 8.01 (d, J = 2.51 Hz, 1H), 7.71-7.72 (m, 1H), 7.27 (dd, J = 10.92, 9.16 Hz, 1H), 5.93 (d, J = 5.02 Hz, 1H), 2.68 (d, J = 5.02 Hz, 3H), 2.47-2.49 (m, 3H), 2.39 (s, 3H). LCMS m/z = 381.3 (M + 1); RT = 1.33, Method 3. |
| 213 | 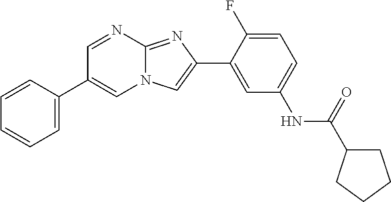 [SMILES: O=C(Nc1ccc(F)c(-c2cn3cc(-c4ccccc4)cnc3n2)c1)C1CCCC1] | ¹H NMR (400 MHz, CD₃OD) δ 9.13 (d, J = 2.5 Hz, 1H), 8.90 (d, J = 2.5 Hz, 1H), 8.27 (d, J = 3.8 Hz, 1H), 8.12 (dd, J = 6.6, 2.7 Hz, 1H), 7.84 (ddd, J = 8.9, 4.5, 2.8 Hz, 1H), 7.78-7.67 (m, 2H), 7.60-7.49 (m, 2H), 7.50-7.41 (m, 1H), 7.19 (dd, J = 11.0, 8.9 Hz, 1H), 2.83 (q, J = 8.0 Hz, 1H), 2.04-1.92 (m, 2H), 1.93-1.73 (m, 5H), 1.67 (dd, J = 7.4, 4.8 Hz, 2H). |
| 214 | 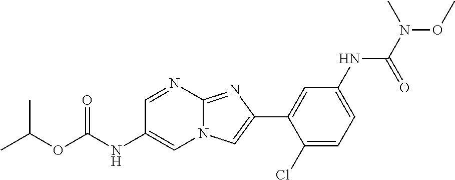 [SMILES: CON(C)C(=O)Nc1ccc(Cl)c(-c2cn3cc(NC(=O)OC(C)C)cnc3n2)c1] | LCMS m/z = 433 (M + 1); RT = 0.92, Method 2. |
| 215 | 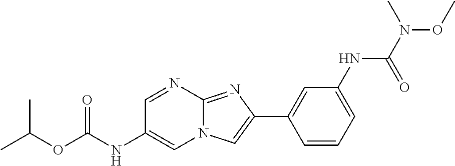 [SMILES: CON(C)C(=O)Nc1cccc(-c2cn3cc(NC(=O)OC(C)C)cnc3n2)c1] | LCMS m/z = 399.1 (M + 1); RT = 0.77, Method 2. |
| 216 | 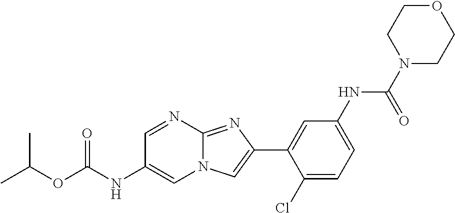 [SMILES: CC(C)OC(=O)Nc1cnc2nc(-c3cc(NC(=O)N4CCOCC4)ccc3Cl)cn2c1] | ¹H NMR (400 MHz, CD₃OD) δ 9.60 (s, 1H), 8.79 (d, J = 2.4 Hz, 1H), 8.50 (s, 1H), 7.95 (d, J = 2.5 Hz, 1H), 7.54 (d, J = 8.7 Hz, 1H), 7.47 (dd, J = 8.9, 2.5 Hz, 1H), 5.06 (hept, J = 6.4 Hz, 1H), 3.72 (dd, J = 5.7, 3.9 Hz, 4H), 3.55 (dd, J = 5.8, 3.9 Hz, 4H), 1.35 (d, J = 6.2 Hz, 6H). LCMS m/z = 459 (M + 1); RT = 0.86, Method 2. |
| 217 | 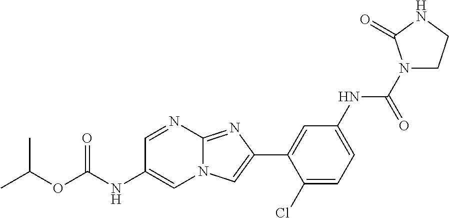 [SMILES: CC(C)OC(=O)Nc1cnc2nc(-c3cc(NC(=O)N4CCNC4=O)ccc3Cl)cn2c1] | LCMS m/z = 457.9 (M + 1); RT = 0.88, Method 2. |
| 218 | 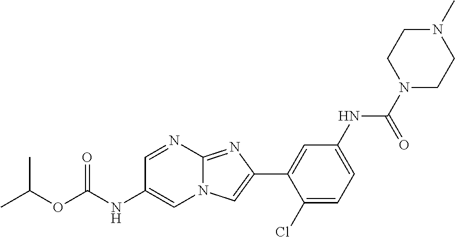 [SMILES: CC(C)OC(=O)Nc1cnc2nc(-c3cc(NC(=O)N4CCN(C)CC4)ccc3Cl)cn2c1] | LCMS m/z = 472 (M + 1); RT = 0.7, Method 2. |
| 219 | 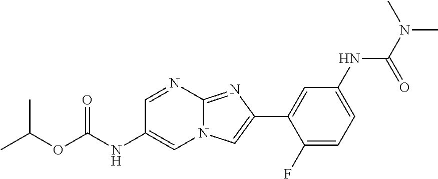 [SMILES: CC(C)OC(=O)Nc1cnc2nc(-c3cc(NC(=O)N(C)C)ccc3F)cn2c1] | LCMS m/z = 401.2 (M + 1); RT = 2.86, Method 1. |
| 220 | 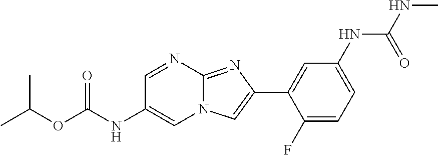 [SMILES: CNC(=O)Nc1ccc(F)c(-c2cn3cc(NC(=O)OC(C)C)cnc3n2)c1] | LCMS m/z = 386.0 (M). RT = 0.87, Method 2. |
| 221 | 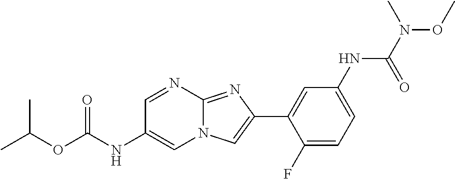 [SMILES: CON(C)C(=O)Nc1ccc(F)c(-c2cn3cc(NC(=O)OC(C)C)cnc3n2)c1] | ¹H NMR (400 MHz, CD₃OD) δ 9.25 (s, 1H), 8.47 (d, J = 2.6 Hz, 1H), 8.17 (d, J = 3.7 Hz, 1H), 8.04 (dd, J = 6.7, 2.8 Hz, 1H), 7.62 (ddd, J = 9.0, 4.3, 2.7 Hz, 1H), 7.16 (dd, J = 10.9, 8.9 Hz, 1H), 5.01 (hept, J = 6.2 Hz, 1H), 3.79 (s, 3H), 3.17 (s, 3H), 1.33 (d, J = 6.3 Hz, 6H). LCMS m/z = 417 (M + 1); RT = 0.88, Method 2. |
| 222 | 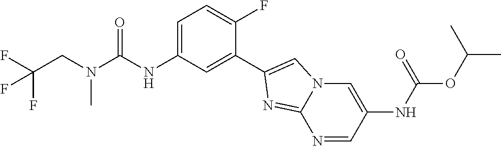 [SMILES: CC(C)OC(=O)Nc1cnc2nc(-c3cc(NC(=O)N(C)CC(F)(F)F)ccc3F)cn2c1] | ¹H NMR (400 MHz, CD₃OD) δ 9.52 (s, 1H), 8.72 (d, J = 2.6 Hz, 1H), 8.40 (d, J = 2.7 Hz, 1H), 8.05 (dd, J = 6.6, 2.7 Hz, 1H), 7.49 (ddd, J = 8.8, 4.4, 2.8 Hz, 1H), 7.29 (dd, J = 10.5, 9.2 Hz, 1H), 5.04 (hept, J = 6.3 Hz, 1H), 4.18 (q, J = 9.2 Hz, 2H), 3.22 (s, 3H), 1.35 (d, J = 6.2 Hz, 6H). LCMS m/z = 469.2 (M + 1); RT = 1.57, Method 3. |
| 223 | 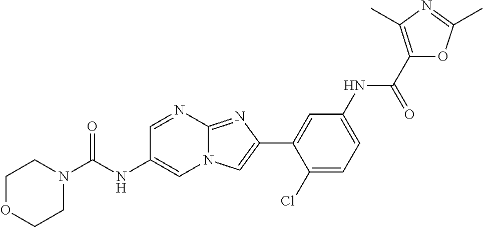 | ¹H NMR (400 MHz, DMSO-d₆) δ 10.34 (s, 1H), 9.28 (d, J = 2.6, 1H), 8.93 (s, 1H), 8.74 (s, 1H), 8.66 (d, J = 6.8, 1H), 8.62 (d, J = 2.6, 1H), 8.37 (d, J = 4.0, 1H), 7.73 (d, J = 21.9, 2H), 7.37-7.27 (m, 1H), 3.78-3.73 (m, 4H), 3.65-3.62 (m, 4H), 2.50 (s, 3H), 2.40 (s, 3H). LCMS m/z = 480.4 (M + 1); RT = 1.39, Method 3. |
| 224 | 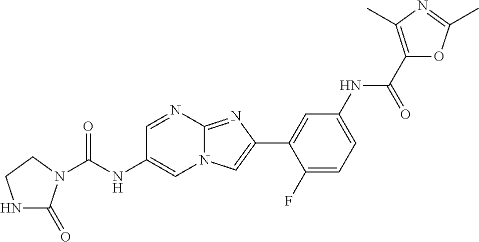 [SMILES: Cc1nc(C)c(C(=O)Nc2ccc(F)c(-c3cn4cc(NC(=O)N5CCNC5=O)cnc4n3)c2)o1] | ¹H NMR (400 MHz, DMSO-d₆) δ 10.46 (s, 1H), 10.33 (s, 1H), 9.38 (d, J = 2.7, 1H), 8.69 (dd, J = 2.8, 6.9, 1H), 8.57 (d, J = 2.7, 1H), 8.37 (d, J = 4.2, 1H), 7.96 (d, J = 7.6, 1H), 7.79 (ddd, J = 2.8, 4.4, 8.9, 1H), 7.31 (dd, J = 9.0, 10.9, 1H), 3.88 (dd, J = 7.3, 9.2, 2H), 3.41 (dd, J = 6.1, 14.2, 2H), 2.50 (s, 3H), 2.40 (s, 3H). LCMS m/z = 479.4 (M + 1); RT = 1.42, Method 3. |
| 225 | 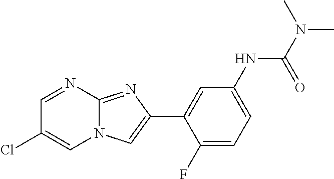 [SMILES: CN(C)C(=O)Nc1ccc(F)c(-c2cn3cc(Cl)cnc3n2)c1] | ¹H NMR (600 MHz, CD₃OD) δ 9.16 (d, J = 2.5 Hz, 1H), 8.69 (d, J = 2.6 Hz, 1H), 8.26 (d, J = 3.3 Hz, 1H), 8.00 (dd, J = 6.6, 2.7 Hz, 1H), 7.50 (ddd, J = 8.8, 4.5, 2.7 Hz, 1H), 7.19 (dd, J = 10.9, 8.8 Hz, 1H), 3.05 (s, 6H). LCMS m/z = 334 (M + 1); RT = 0.75, Method 2. |
| 226 | 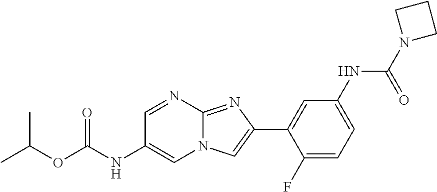 [SMILES: CC(C)OC(=O)Nc1cnc2nc(-c3cc(NC(=O)N4CCC4)ccc3F)cn2c1] | ¹H NMR (400 MHz, CD₃OD) δ 9.25 (s, 1H), 8.46 (d, J = 2.6 Hz, 1H), 8.18 (d, J = 3.8 Hz, 1H), 7.94 (dd, J = 6.6, 2.8 Hz, 1H), 7.62 (ddd, J = 8.9, 4.4, 2.8 Hz, 1H), 7.13 (dd, J = 10.9, 9.0 Hz, 1H), 5.02 (hept, J = 6.2 Hz, 1H), 4.17- 4.04 (m, 4H), 2.33 (p, J = 7.6 Hz, 2H), 1.34 (d, J = 6.3 Hz, 6H). LCMS m/z = 413 (M + 1); RT = 0.83, Method 2. |
| 227 | 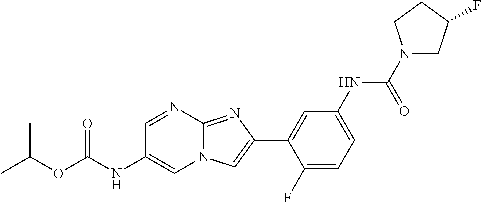 [SMILES: CC(C)OC(=O)Nc1cnc2nc(-c3cc(NC(=O)N4CC[C@H](F)C4)ccc3F)cn2c1] | LCMS m/z = 445.1 (M + 1); RT = 0.86, Method 2. |
| 228 | 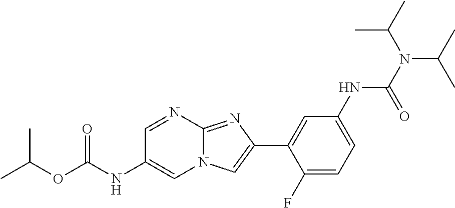 [SMILES: CC(C)OC(=O)Nc1cnc2nc(-c3cc(NC(=O)N(C(C)C)C(C)C)ccc3F)cn2c1] | ¹H NMR (400 MHz, CD₃OD) δ 9.51 (s, 1H), 8.70 (d, J = 2.5 Hz, 1H), 8.39 (d, J = 2.8 Hz, 1H), 7.97 (dd, J = 6.6, 2.7 Hz, 1H), 7.40 (ddd, J = 8.8, 4.5, 2.7 Hz, 1H), 7.25 (dd, J = 10.7, 9.0 Hz, 1H), 5.08-5.00 (m, 1H), 3.98-3.88 (m, 2H), 1.41-1.32 (m, 18H). LCMS m/z = 457.1 (M + 1); RT = 1.04, Method 2. |
| 229 | 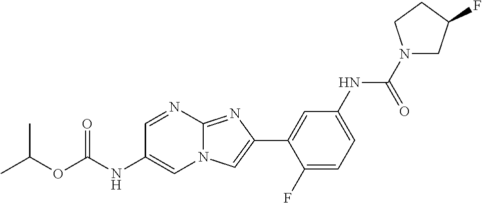 [SMILES: CC(C)OC(=O)Nc1cnc2nc(-c3cc(NC(=O)N4CC[C@@H](F)C4)ccc3F)cn2c1] | ¹H NMR (400 MHz, CD₃OD) δ 9.47 (s, 1H), 8.67 (d, J = 2.6 Hz, 1H), 8.35 (d, J = 3.0 Hz, 1H), 8.04 (dd, J = 6.6, 2.7 Hz, 1H), 7.53 (ddd, J = 8.9, 4.4, 2.7 Hz, 1H), 7.25 (dd, J = 10.7, 9.0 Hz, 1H), 5.46-5.20 (m, 1H), 5.11-4.99 (m, 1H), 3.89-3.67 (m, 2H), 3.69-3.49 (m, 2H), 2.38-2.12 (m, 2H), 1.35 (d, J = 6.3 Hz, 6H). LCMS m/z = 445 (M + 1); RT = 0.86, Method 2. |
| 230 | 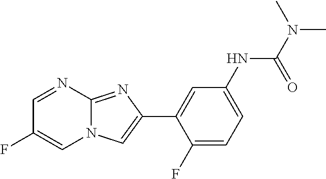 [SMILES: CN(C)C(=O)Nc1ccc(F)c(-c2cn3cc(F)cnc3n2)c1] | ¹H NMR (600 MHz, CD₃OD) δ 9.20 (dd, J = 3.6, 2.8 Hz, 1H), 8.89 (d, J = 2.8 Hz, 1H), 8.36 (d, J = 3.1 Hz, 1H), 8.02 (dd, J = 6.5, 2.8 Hz, 1H), 7.50 (ddd, J = 8.9, 4.4, 2.7 Hz, 1H), 7.24 (dd, J = 10.7, 9.0 Hz, 1H), 3.06 (s, 6H). LCMS m/z = 318.1 (M + 1); RT = 0.69, Method 4. |
| 231 | 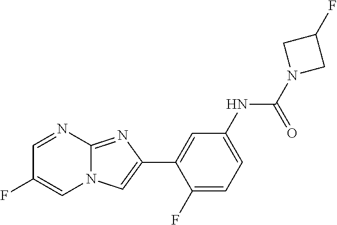 [SMILES: O=C(Nc1ccc(F)c(-c2cn3cc(F)cnc3n2)c1)N1CC(F)C1] | ¹H NMR (600 MHz, CD₃OD) δ 9.10 (dd, J = 3.8, 2.8 Hz, 1H), 8.78 (d, J = 2.9 Hz, 1H), 8.30 (d, J = 3.3 Hz, 1H), 8.05 (dd, J = 6.5, 2.8 Hz, 1H), 7.60 (ddd, J = 8.9, 4.4, 2.8 Hz, 1H), 7.20 (dd, J = 10.9, 9.0 Hz, 1H), 5.37 (dtt, J = 57.2, 6.2, 3.2 Hz, 1H), 4.39 (dddd, J = 20.8, 10.2, 6.0, 1.4 Hz, 2H), 4.14 (dddd, J = 24.5, 10.3, 3.2, 1.5 Hz, 2H). LCMS m/z = 348 (M + 1); RT = 0.72, Method 2. |
| 232 | 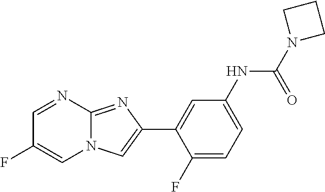 [SMILES: O=C(Nc1ccc(F)c(-c2cn3cc(F)cnc3n2)c1)N1CCC1] | ¹H NMR (600 MHz, CD₃OD) δ 9.06 (dd, J = 3.8, 2.8 Hz, 1H), 8.74 (d, J = 2.8 Hz, 1H), 8.27 (d, J = 3.6 Hz, 1H), 8.01 (dd, J = 6.5, 2.8 Hz, 1H), 7.61 (ddd, J = 8.9, 4.4, 2.8 Hz, 1H), 7.17 (dd, J = 10.9, 8.9 Hz, 1H), 4.16-4.07 (m, 4H), 2.33 (tt, J = 7.7, 6.9 Hz, 2H). LCMS m/z = 330 (M + 1); RT = 0.7, Method 2. |
| 233 | 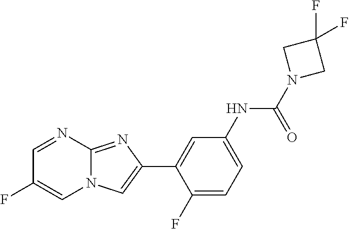 [SMILES: O=C(Nc1ccc(F)c(-c2cn3cc(F)cnc3n2)c1)N1CC(F)(F)C1] | ¹H NMR (600 MHz, CD₃OD) δ 9.04 (dd, J = 3.8, 2.8 Hz, 1H), 8.72 (d, J = 2.9 Hz, 1H), 8.27 (d, J = 3.6 Hz, 1H), 8.06 (dd, J = 6.5, 2.8 Hz, 1H), 7.63 (ddd, J = 8.9, 4.4, 2.8 Hz, 1H), 7.20 (dd, J = 11.0, 8.9 Hz, 1H), 4.43 (t, J = 12.3 Hz, 4H). LCMS m/z = 366 (M + 1); RT = 0.79, Method 2. |
| 234 | 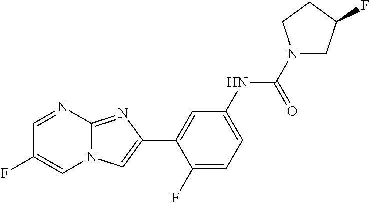 [SMILES: O=C(Nc1ccc(F)c(-c2cn3cc(F)cnc3n2)c1)N1CC[C@@H](F)C1] | ¹H NMR (600 MHz, CD₃OD) δ 9.17 (dd, J = 3.7, 2.8 Hz, 1H), 8.85 (d, J = 2.8 Hz, 1H), 8.34 (d, J = 3.1 Hz, 1H), 8.06 (dd, J = 6.5, 2.7 Hz, 1H), 7.56 (ddd, J = 8.9, 4.4, 2.8 Hz, 1H), 7.23 (dd, J = 10.8, 8.9 Hz, 1H), 5.34 (dtd, J = 52.9, 3.7, 1.4 Hz, 1H), 3.80 (ddd, J = 24.2, 12.7, 2.0 Hz, 1H), 3.74 (td, J = 9.7, 1.5 Hz, 1H), 3.67 (ddd, J = 38.2, 12.7, 3.5 Hz, 1H), 3.59 (ddd, J = 11.0, 9.9, 6.7 Hz, 1H), 2.36- 2.27 (m, 1H), 2.26-2.10 (m, 1H). LCMS m/z = 362.1 (M + 1); RT = 0.75, Method 4. |
| 235 | 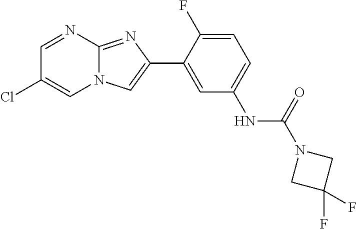 [SMILES: O=C(Nc1ccc(F)c(-c2cn3cc(Cl)cnc3n2)c1)N1CC(F)(F)C1] | ¹H NMR (600 MHz, CD₃OD) δ 9.14 (d, J = 2.5 Hz, 1H), 8.65 (d, J = 2.5 Hz, 1H), 8.26 (d, J = 3.4 Hz, 1H), 8.09 (dd, J = 6.5, 2.8 Hz, 1H), 7.62 (ddd, J = 8.9, 4.4, 2.8 Hz, 1H), 7.21 (dd, J = 10.9, 8.9 Hz, 1H), 4.43 (t, J = 12.3 Hz, 4H). LCMS m/z = 381.9 (M + 1); RT = 0.86, Method 2. |
| 236 | 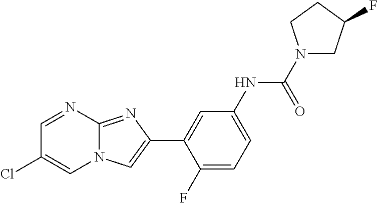 [SMILES: O=C(Nc1ccc(F)c(-c2cn3cc(Cl)cnc3n2)c1)N1CC[C@@H](F)C1] | ¹H NMR (600 MHz, CD₃OD) δ 9.16 (d, J = 2.5 Hz, 1H), 8.68 (d, J = 2.5 Hz, 1H), 8.26 (d, J = 3.2 Hz, 1H), 8.05 (dd, J = 6.6, 2.8 Hz, 1H), 7.57 (ddd, J = 8.9, 4.4, 2.8 Hz, 1H), 7.20 (dd, J = 10.9, 8.9 Hz, 1H), 5.41-5.25 (m, 1H), 3.79 (ddd, J = 24.3, 12.8, 2.0 Hz, 1H), 3.74 (td, J = 9.7, 1.5 Hz, 1H), 3.72-3.61 (m, 1H), 3.58 (ddd, J = 11.1, 9.9, 6.7 Hz, 1H), 2.36-2.26 (m, 1H), 2.26-2.09 (m, 1H). LCMS m/z = 378 (M + 1); RT = 0.8, Method 2. |
| 237 | 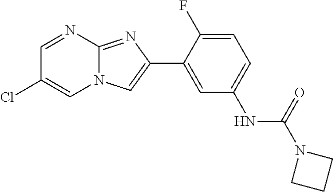 [SMILES: O=C(Nc1ccc(F)c(-c2cn3cc(Cl)cnc3n2)c1)N1CCC1] | LCMS m/z = 346 (M + 1); RT = 0.78, Method 2. |
| 238 | 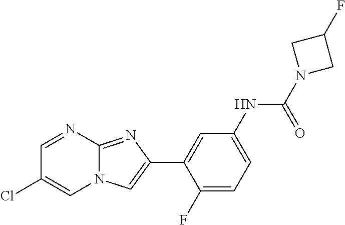 [SMILES: O=C(Nc1ccc(F)c(-c2cn3cc(Cl)cnc3n2)c1)N1CC(F)C1] | LCMS m/z = 364 (M + 1); RT = 0.82, Method 4. |
| 239 | 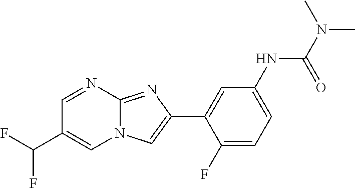 [SMILES: CN(C)C(=O)Nc1ccc(F)c(-c2cn3cc(C(F)F)cnc3n2)c1] | ¹H NMR (400 MHz, DMSO-d₆) δ 9.36 (s, 1H), 8.76 (s, 1H), 8.52 (s, 1H), 8.28-8.44 (m, 2H), 7.63 (dd, J = 8.03, 3.76 Hz, 1H), 7.03-7.49 (m, 2H), 2.96 (s, 6H). LCMS m/z = 350.1 (M + 1); RT = 0.77, Method 4. |
| 240 | 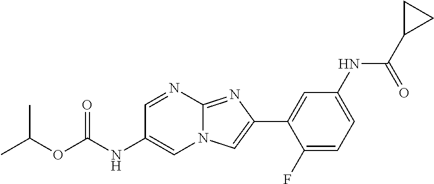 [SMILES: CC(C)OC(=O)Nc1cnc2nc(-c3cc(NC(=O)C4CC4)ccc3F)cn2c1] | LCMS m/z = 398.2 (M + 1); RT = 3.07, Method 1. |
| 241 | 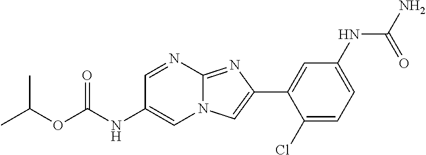 | LCMS m/z = 436 (M + 1); RT = 0.89, Method 2. |
| 242 | 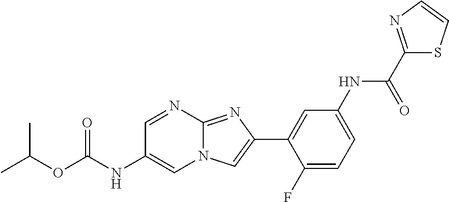 [SMILES: CC(C)OC(=O)Nc1cnc2nc(-c3cc(NC(=O)c4nccs4)ccc3F)cn2c1] | ¹H NMR (400 MHz, CD₃OD) δ 9.48 (s, 1H), 8.68 (d, J = 2.5 Hz, 1H), 8.39 (d, J = 2.9 Hz, 1H), 8.36 (dd, J = 6.5, 2.6 Hz, 1H), 8.05 (d, J = 2.9 Hz, 1H), 7.94 (d, J = 3.0 Hz, 1H), 7.87 (ddd, J = 8.9, 4.4, 2.8 Hz, 1H), 7.34 (dd, J = 10.7, 9.1 Hz, 1H), 5.04 (hept, J = 6.3 Hz, 1H), 1.34 (d, J = 6.2 Hz, 6H). LCMS m/z = 441 (M + 1); RT = 0.96, Method 2. |
| 243 | 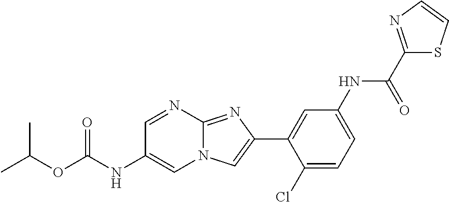 [SMILES: CC(C)OC(=O)Nc1cnc2nc(-c3cc(NC(=O)c4nccs4)ccc3Cl)cn2c1] | ¹H NMR (400 MHz, CD₃OD) δ 9.47 (d, J = 4.2 Hz, 1H), 8.71-8.62 (m, 1H), 8.48 (d, J = 2.3 Hz, 1H), 8.33 (t, J = 2.3 Hz, 1H), 8.04 (dd, J = 3.0, 1.3 Hz, 1H), 7.97-7.91 (m, 1H), 7.88 (d, J = 8.7 Hz, 1H), 7.64-7.53 (m, 1H), 5.04 (heptd, J = 6.2, 1.2 Hz, 1H), 1.34 (d, J = 6.3 Hz, 6H). LCMS m/z = 456.9 (M + 1); RT = 1, Method 2. |
| 244 | 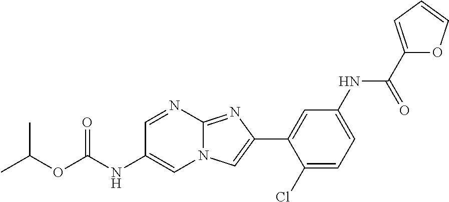 [SMILES: CC(C)OC(=O)Nc1cnc2nc(-c3cc(NC(=O)c4ccco4)ccc3Cl)cn2c1] | ¹H NMR (400 MHz, CD₃OD) δ 9.62- 9.48 (m, 1H), 8.76 (d, J = 2.5 Hz, 1H), 8.52 (s, 1H), 8.30 (d, J = 2.5 Hz, 1H), 7.82 (dd, J = 8.8, 2.6 Hz, 1H), 7.78 (d, J = 1.6 Hz, 1H), 7.62 (d, J = 8.8 Hz, 1H), 7.32 (d, J = 3.3 Hz, 1H), 6.67 (dd, J = 3.6, 1.7 Hz, 1H), 5.05 (hept, J = 6.3 Hz, 1H), 1.35 (d, J = 6.2 Hz, 6H). LCMS m/z = 439.9 (M + 1); RT = 0.95, Method 2. |
| 245 | 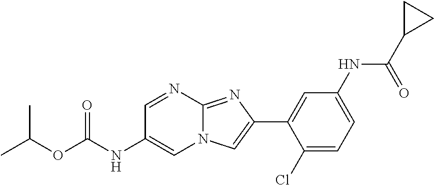 [SMILES: CC(C)OC(=O)Nc1cnc2nc(-c3cc(NC(=O)C4CC4)ccc3Cl)cn2c1] | LCMS m/z = 414.2 (M + 1); RT = 3.15, Method 1. |
| 246 | 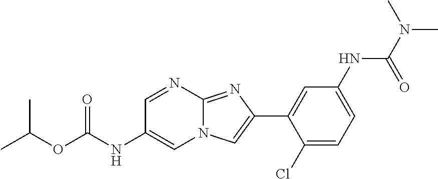 [SMILES: CC(C)OC(=O)Nc1cnc2nc(-c3cc(NC(=O)N(C)C)ccc3Cl)cn2c1] | LCMS m/z = 417 (M + 1); RT = 0.85, Method 2. |
| 247 | 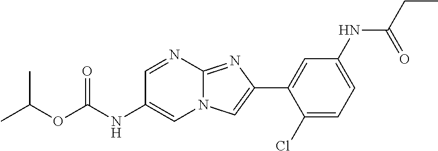 [SMILES: CCC(=O)Nc1ccc(Cl)c(-c2cn3cc(NC(=O)OC(C)C)cnc3n2)c1] | LCMS m/z = 402.2 (M + 1); RT = 3.1, Method 1. |
| 248 | 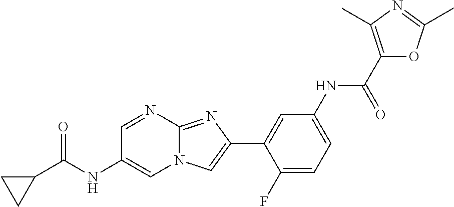 [SMILES: Cc1nc(C)c(C(=O)Nc2ccc(F)c(-c3cn4cc(NC(=O)C5CC5)cnc4n3)c2)o1] | ¹H NMR (400 MHz, DMSO-d₆) δ 10.68 (s, 1H), 10.33 (s, 1H), 9.55 (d, J = 2.6, 1H), 8.69 (dd, J = 2.8, 6.9, 1H), 8.54 (d, J = 2.6, 1H), 8.37 (d, J = 4.2, 1H), 7.78 (ddd, J = 2.8, 4.4, 8.9, 1H), 7.30 (dd, J = 9.0, 11.0, 1H), 2.50 (s, 3H), 2.41 (d, J = 8.0, 3H), 1.90-1.79 (m, 1H), 0.88 (dd, J = 6.3, 10.9, 4H). LCMS m/z = 435.4 (M + 1); RT = 1.48, Method 3. |
| 249 | 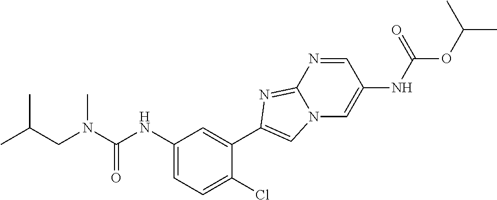 [SMILES: CC(C)CN(C)C(=O)Nc1ccc(Cl)c(-c2cn3cc(NC(=O)OC(C)C)cnc3n2)c1] | ¹H NMR (400 MHz, CD₃OD) δ 9.58 (s, 1H), 8.78 (d, J = 2.6 Hz, 1H), 8.50 (s, 1H), 7.95 (d, J = 2.5 Hz, 1H), 7.52 (d, J = 8.7 Hz, 1H), 7.47 (dd, J = 8.8, 2.5 Hz, 1H), 5.05 (hept, J = 6.2 Hz, 1H), 3.25 (d, J = 7.5 Hz, 2H), 3.06 (s, 3H), 2.02 (dp, J = 13.8, 6.8 Hz, 1H), 1.35 (d, J = 6.2 Hz, 6H), 0.94 (d, J = 6.7 Hz, 6H). LCMS m/z = 459.2 (M + 1); RT = 3.28, Method 1. |
| 250 | 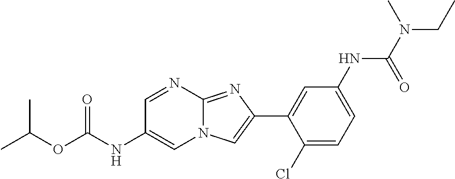 [SMILES: CCN(C)C(=O)Nc1ccc(Cl)c(-c2cn3cc(NC(=O)OC(C)C)cnc3n2)c1] | ¹H NMR (400 MHz, CD₃OD) δ 9.56 (s, 1H), 8.75 (d, J = 2.5 Hz, 1H), 8.49 (s, 1H), 7.95 (d, J = 2.4 Hz, 1H), 7.56-7.44 (m, 2H), 5.05 (hept, J = 6.2 Hz, 1H), 3.46 (q, J = 7.1 Hz, 2H), 3.04 (s, 3H), 1.35 (d, J = 6.3 Hz, 6H), 1.19 (t, J = 7.2 Hz, 3H). LCMS m/z = 431.2 (M + 1); RT = 3.04, Method 1. |
| 251 | 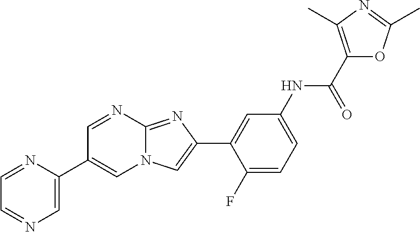 [SMILES: Cc1nc(C)c(C(=O)Nc2ccc(F)c(-c3cn4cc(-c5cnccn5)cnc4n3)c2)o1] | ¹H NMR (400 MHz, CD₃OD) δ 9.79 (d, J = 2.4 Hz, 1H), 9.53 (d, J = 2.2 Hz, 1H), 9.32 (d, J = 1.5 Hz, 1H), 8.84-8.76 (m, 1H), 8.69 (d, J = 2.5 Hz, 1H), 8.49 (d, J = 3.1 Hz, 1H), 8.40 (dd, J = 6.6, 2.6 Hz, 1H), 7.82 (ddd, J = 8.8, 4.4, 2.7 Hz, 1H), 7.35 (dd, J = 10.8, 9.0 Hz, 1H), 2.57 (s, 3H), 2.48 (s, 3H). LCMS m/z = 430.4 (M + 1); RT = 1.57, Method 3. |
| 252 | 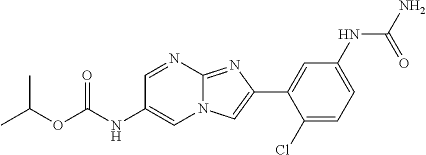 [SMILES: CC(C)OC(=O)Nc1cnc2nc(-c3cc(NC(N)=O)ccc3Cl)cn2c1] | LCMS m/z = 389 (M + 1); RT = 0.78, Method 2. |
| 253 | 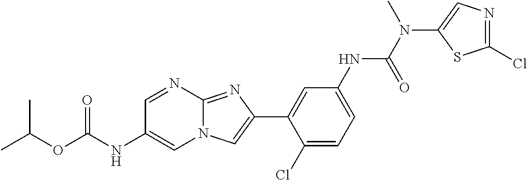 | LCMS m/z = 000 (M + 1); RT = 0.00, Method X. LIANGFA3-001-EXP050-007-1 |
| 254 | 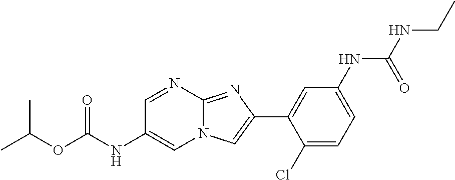 [SMILES: CCNC(=O)Nc1ccc(Cl)c(-c2cn3cc(NC(=O)OC(C)C)cnc3n2)c1] | LCMS m/z = 417 (M + 1); RT = 0.87, Method 2. |
| 255 | 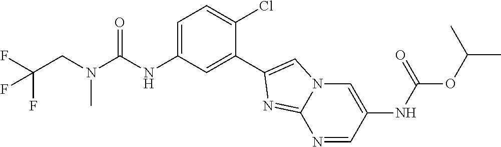 [SMILES: CC(C)OC(=O)Nc1cnc2nc(-c3cc(NC(=O)N(C)CC(F)(F)F)ccc3Cl)cn2c1] | ¹H NMR (400 MHz, CD₃OD) δ 9.60- 9.49 (m, 1H), 8.74 (d, J = 2.5 Hz, 1H), 8.49 (s, 1H), 7.99 (d, J = 2.3 Hz, 1H), 7.58-7.47 (m, 2H), 5.05 (hept, J = 6.0 Hz, 1H), 4.17 (q, J = 9.2 Hz, 2H), 3.21 (s, 3H), 1.34 (d, J = 6.3 Hz, 6H). LCMS m/z = 485.2 (M + 1); RT = 3.16, Method 1. |
| 256 | 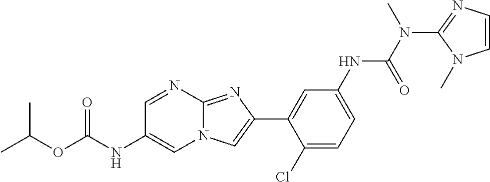 [SMILES: CC(C)OC(=O)Nc1cnc2nc(-c3cc(NC(=O)N(C)c4nccn4C)ccc3Cl)cn2c1] | LCMS m/z = 483 (M + 1); RT = 0.72, Method 2. |
| 257 | 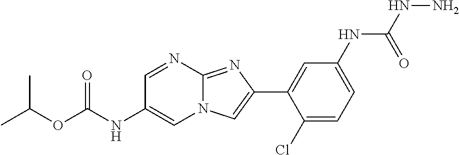 [SMILES: CC(C)OC(=O)Nc1cnc2nc(-c3cc(NC(=O)NN)ccc3Cl)cn2c1] | LCMS m/z = 403.9 (M + 1); RT = 0.75, Method 2. |
| 258 | 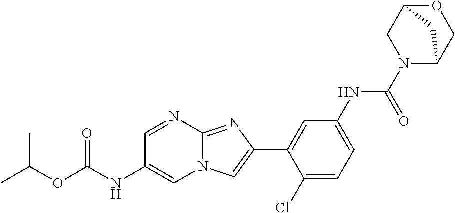 [SMILES: CC(C)OC(=O)Nc1cnc2nc(-c3cc(NC(=O)N4C[C@@H]5C[C@H]4CO5)ccc3Cl)cn2c1] | LCMS m/z = 471 (M + 1); RT = 0.83, Method 2. |
| 259 | 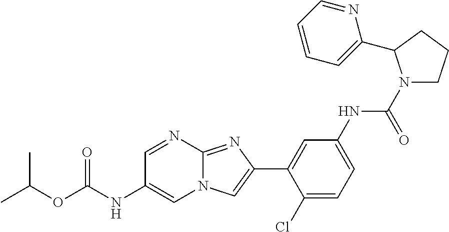 [SMILES: CC(C)OC(=O)Nc1cnc2nc(-c3cc(NC(=O)N4CCCC4c4ccccn4)ccc3Cl)cn2c1] | LCMS m/z = 520 (M + 1); RT = 0.78, Method 2. |
| 260 | 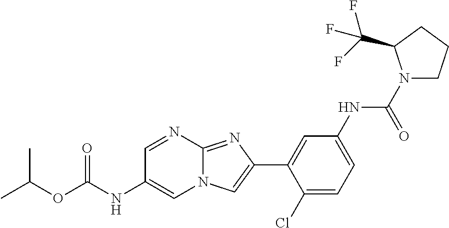 [SMILES: CC(C)OC(=O)Nc1cnc2nc(-c3cc(NC(=O)N4CCC[C@@H]4C(F)(F)F)ccc3Cl)cn2c1] | LCMS m/z = 511 (M + 1); RT = 1.04, Method 2. |
| 261 | 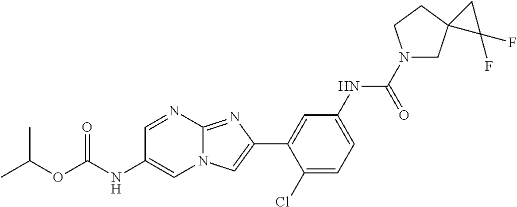 [SMILES: CC(C)OC(=O)Nc1cnc2nc(-c3cc(NC(=O)N4CCC5(C4)CC5(F)F)ccc3Cl)cn2c1] | LCMS m/z = 505 (M + 1); RT = 0.99, Method 2. |
| 262 | 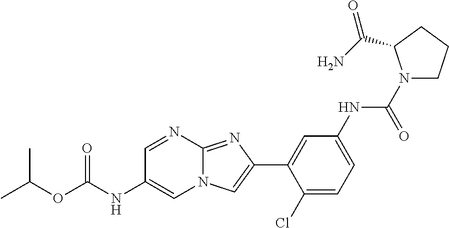 [SMILES: CC(C)OC(=O)Nc1cnc2nc(-c3cc(NC(=O)N4CCC[C@H]4C(N)=O)ccc3Cl)cn2c1] | LCMS m/z = 486 (M + 1); RT = 0.77, Method 2. |
| 263 | 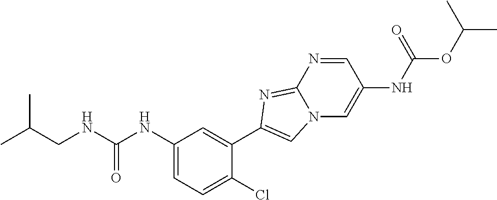 [SMILES: CC(C)CNC(=O)Nc1ccc(Cl)c(-c2cn3cc(NC(=O)OC(C)C)cnc3n2)c1] | LCMS m/z = 445 (M + 1); RT = 0.98, Method 2. |
| 264 | 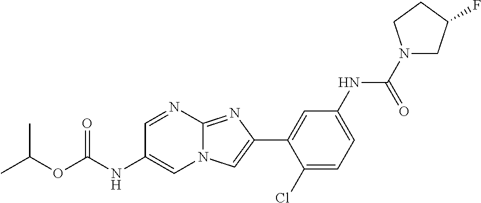 [SMILES: CC(C)OC(=O)Nc1cnc2nc(-c3cc(NC(=O)N4CC[C@H](F)C4)ccc3Cl)cn2c1] | LCMS m/z = 461 (M + 1); RT = 0.88, Method 2. |
| 265 | 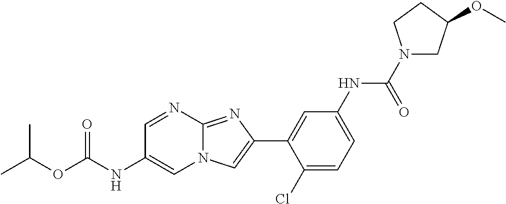 [SMILES: CO[C@@H]1CCN(C(=O)Nc2ccc(Cl)c(-c3cn4cc(NC(=O)OC(C)C)cnc4n3)c2)C1] | LCMS m/z = 473 (M + 1); RT = 0.87, Method 2. |
| 266 | 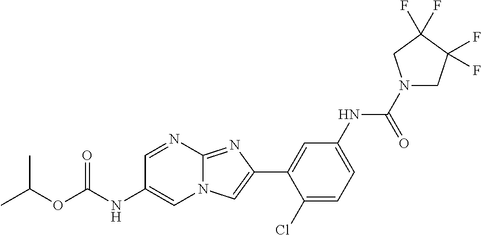 [SMILES: CC(C)OC(=O)Nc1cnc2nc(-c3cc(NC(=O)N4CC(F)(F)C(F)(F)C4)ccc3Cl)cn2c1] | LCMS m/z = 515 (M + 1); RT = 1.04, Method 2. |
| 267 | 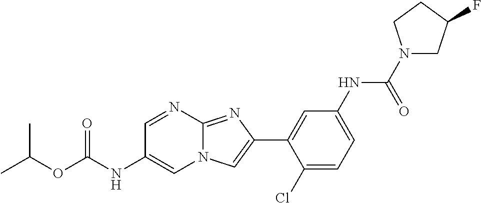 [SMILES: CC(C)OC(=O)Nc1cnc2nc(-c3cc(NC(=O)N4CC[C@@H](F)C4)ccc3Cl)cn2c1] | LCMS m/z = 460.9 (M + 1); RT = 0.88, Method 2. |
| 268 | 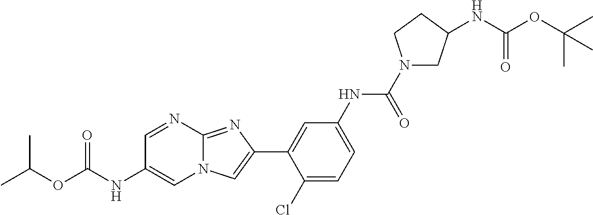 [SMILES: CC(C)OC(=O)Nc1cnc2nc(-c3cc(NC(=O)N4CCC(NC(=O)OC(C)(C)C)C4)ccc3Cl)cn2c1] | LCMS m/z = 558.1 (M + 1); RT = 0.99, Method 2. |
| 269 | 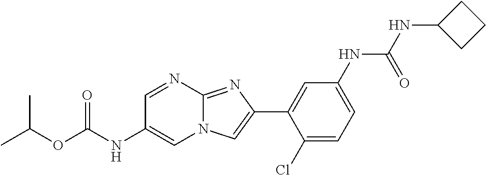 [SMILES: CC(C)OC(=O)Nc1cnc2nc(-c3cc(NC(=O)NC4CCC4)ccc3Cl)cn2c1] | LCMS m/z = 443 (M + 1); RT = 0.94, Method 2. |
| 270 | 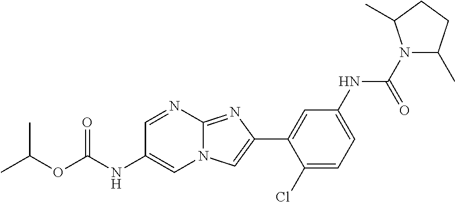 [SMILES: CC(C)OC(=O)Nc1cnc2nc(-c3cc(NC(=O)N4C(C)CCC4C)ccc3Cl)cn2c1] | ¹H NMR (400 MHz, CD₃OD) δ 9.56 (s, 1H), 8.75 (d, J = 2.5 Hz, 1H), 8.50 (d, J = 3.5 Hz, 1H), 8.06-7.91 (m, 1H), 7.57-7.42 (m, 2H), 5.06 (hept, J = 6.2 Hz, 1H), 4.23 (p, J = 6.4 Hz, 2H), 2.27 (s, 2H), 1.66 (d, J = 5.8 Hz, 2H), 1.35 (d, J = 6.2 Hz, 6H), 1.22 (d, J = 6.3 Hz, 6H). LCMS m/z = 471 (M + 1); RT = 1, Method 2. |
| 271 | 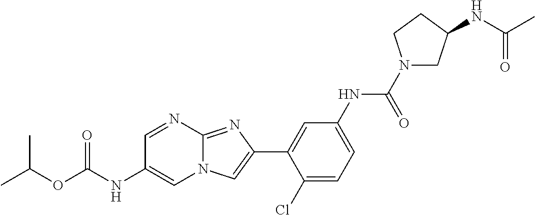 [SMILES: CC(=O)N[C@@H]1CCN(C(=O)Nc2ccc(Cl)c(-c3cn4cc(NC(=O)OC(C)C)cnc4n3)c2)C1] | LCMS m/z = 500 (M + 1); RT = 0.79, Method 2. |
| 272 | 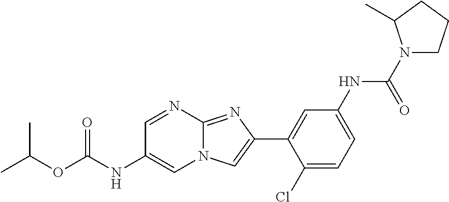 [SMILES: CC(C)OC(=O)Nc1cnc2nc(-c3cc(NC(=O)N4CCCC4C)ccc3Cl)cn2c1] | LCMS m/z = 457 (M + 1); RT = 0.95, Method 2. |
| 273 | 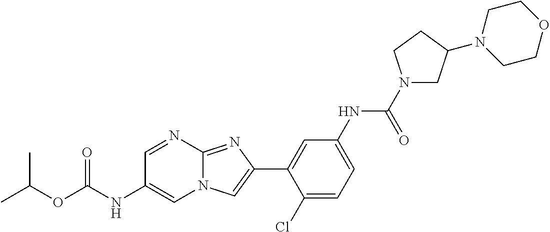 [SMILES: CC(C)OC(=O)Nc1cnc2nc(-c3cc(NC(=O)N4CCC(N5CCOCC5)C4)ccc3Cl)cn2c1] | LCMS m/z = 528.1 (M + 1); RT = 0.7, Method 2. |
| 274 | 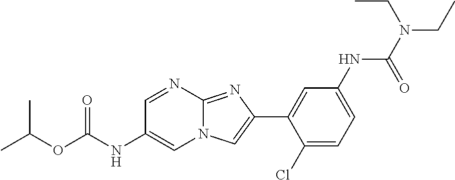 [SMILES: CCN(CC)C(=O)Nc1ccc(Cl)c(-c2cn3cc(NC(=O)OC(C)C)cnc3n2)c1] | LCMS m/z = 445 (M + 1); RT = 0.94, Method 2. |
| 275 | 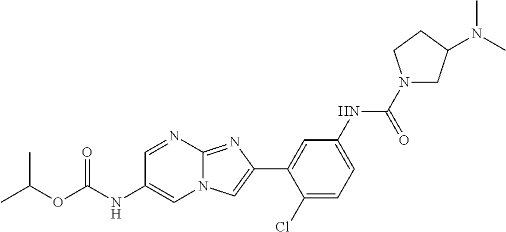 [SMILES: CC(C)OC(=O)Nc1cnc2nc(-c3cc(NC(=O)N4CCC(N(C)C)C4)ccc3Cl)cn2c1] | LCMS m/z = 486 (M + 1); RT = 0.7, Method 2. |
| 276 | 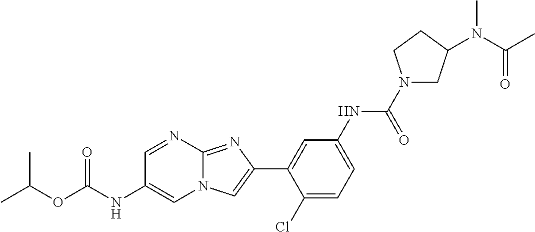 [SMILES: CC(=O)N(C)C1CCN(C(=O)Nc2ccc(Cl)c(-c3cn4cc(NC(=O)OC(C)C)cnc4n3)c2)C1] | LCMS m/z = 514.1 (M + 1); RT = 0.83, Method 2. |
| 277 | 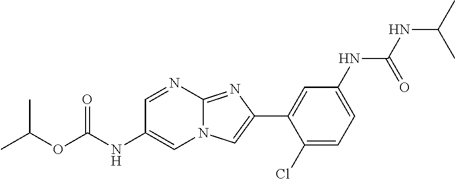 [SMILES: CC(C)NC(=O)Nc1ccc(Cl)c(-c2cn3cc(NC(=O)OC(C)C)cnc3n2)c1] | ¹H NMR (400 MHz, CD₃OD) δ 9.58 (s, 1H), 8.77 (d, J = 2.5 Hz, 1H), 8.50 (s, 1H), 7.97 (d, J = 2.6 Hz, 1H), 7.50 (d, J = 8.7 Hz, 1H), 7.41 (dd, J = 8.7, 2.6 Hz, 1H), 5.05 (hept, J = 6.3 Hz, 1H), 3.90 (hept, J = 6.5 Hz, 1H), 1.35 (d, J = 6.2 Hz, 6H), 1.19 (d, J = 6.5 Hz, 6H). LCMS m/z = 431 (M + 1); RT = 0.93, Method 2. |
| 278 | 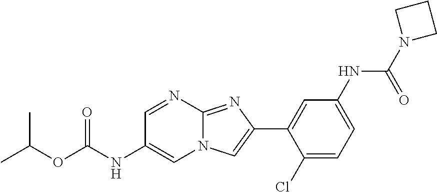 [SMILES: CC(C)OC(=O)Nc1cnc2nc(-c3cc(NC(=O)N4CCC4)ccc3Cl)cn2c1] | LCMS m/z = 429 (M + 1); RT = 0.87, Method 2. |
| 279 | 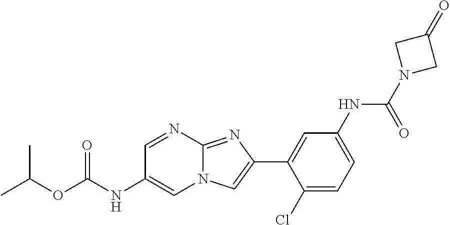 [SMILES: CC(C)OC(=O)Nc1cnc2nc(-c3cc(NC(=O)N4CC(=O)C4)ccc3Cl)cn2c1] | LCMS m/z = 443 (M + 1); RT = 0.86, Method 2. |
| 280 | 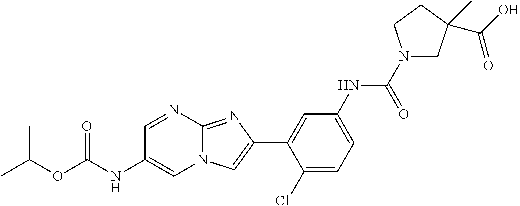 [SMILES: CC(C)OC(=O)Nc1cnc2nc(-c3cc(NC(=O)N4CCC(C)(C(=O)O)C4)ccc3Cl)cn2c1] | LCMS m/z = 501 (M + 1); RT = 0.86, Method 2. |
| 281 | 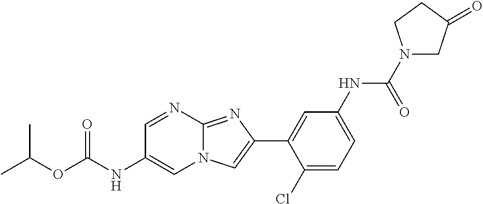 [SMILES: CC(C)OC(=O)Nc1cnc2nc(-c3cc(NC(=O)N4CCC(=O)C4)ccc3Cl)cn2c1] | LCMS m/z = 457 (M + 1); RT = 0.84, Method 2. |
| 282 | 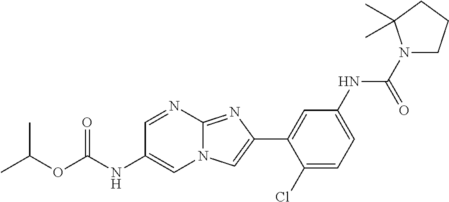 [SMILES: CC(C)OC(=O)Nc1cnc2nc(-c3cc(NC(=O)N4CCCC4(C)C)ccc3Cl)cn2c1] | LCMS m/z = 471.1 (M + 1); RT = 1.04, Method 2. |
| 283 | 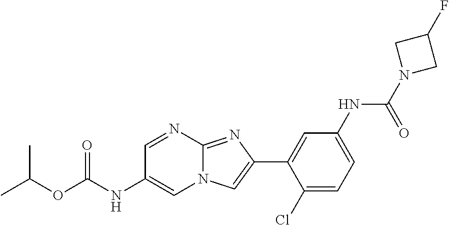 [SMILES: CC(C)OC(=O)Nc1cnc2nc(-c3cc(NC(=O)N4CC(F)C4)ccc3Cl)cn2c1] | ¹H NMR (400 MHz, CD₃OD) δ 9.54 (s, 1H), 8.74 (d, J = 2.7 Hz, 1H), 8.48 (s, 1H), 8.03 (d, J = 2.3 Hz, 1H), 7.56-7.49 (m, 2H), 5.37 (dtt, J = 57.0, 6.1, 3.1 Hz, 1H), 5.11-4.99 (m, 1H), 4.39 (dddd, J = 20.9, 10.3, 6.1, 1.4 Hz, 2H), 4.14 (dddd, J = 24.6, 10.3, 3.1, 1.4 Hz, 2H), 1.34 (d, J = 6.3 Hz, 6H). LCMS m/z = 447 (M + 1); RT = 0.88, Method 2. |
| 284 | 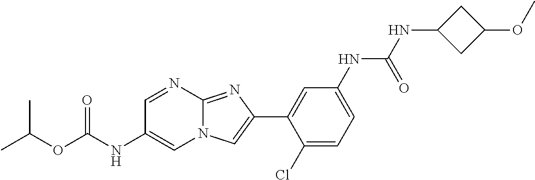 [SMILES: COC1CC(NC(=O)Nc2ccc(Cl)c(-c3cn4cc(NC(=O)OC(C)C)cnc4n3)c2)C1] | LCMS m/z = 473 (M + 1); RT = 0.89, Method 2. |
| 285 | 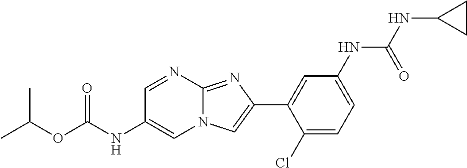 [SMILES: CC(C)OC(=O)Nc1cnc2nc(-c3cc(NC(=O)NC4CC4)ccc3Cl)cn2c1] | LCMS m/z = 429 (M + 1); RT = 0.88, Method 2. |
| 286 | 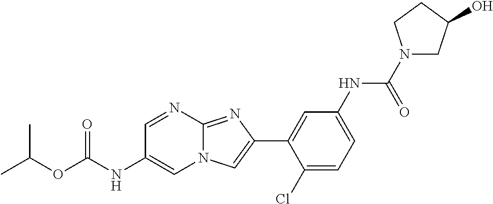 [SMILES: CC(C)OC(=O)Nc1cnc2nc(-c3cc(NC(=O)N4CC[C@@H](O)C4)ccc3Cl)cn2c1] | LCMS m/z = 459 (M + 1); RT = 0.79, Method 2. |
| 287 | 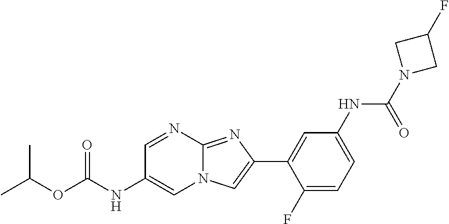 [SMILES: CC(C)OC(=O)Nc1cnc2nc(-c3cc(NC(=O)N4CC(F)C4)ccc3F)cn2c1] | ¹H NMR (400 MHz, CD₃OD) δ 9.51 (s, 1H), 8.71 (d, J = 2.5 Hz, 1H), 8.38 (d, J = 2.8 Hz, 1H), 8.07 (dd, J = 6.6, 2.7 Hz, 1H), 7.52 (ddd, J = 8.9, 4.4, 2.7 Hz, 1H), 7.26 (dd, J = 10.6, 9.0 Hz, 1H), 5.38 (dtt, J = 57.1, 6.1, 3.2 Hz, 1H), 5.04 (hept, J = 6.3 Hz, 1H), 4.40 (dddd, J = 20.8, 10.3, 6.0, 1.3 Hz, 2H), 4.15 (dddd, J = 24.5, 10.4, 3.1, 1.3 Hz, 2H), 1.35 (d, J = 6.3 Hz, 6H). LCMS m/z = 431.2 (M + 1); RT = 2.92, Method 1. |
| 288 | 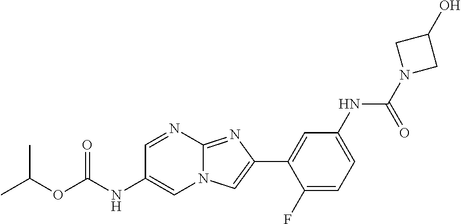 [SMILES: CC(C)OC(=O)Nc1cnc2nc(-c3cc(NC(=O)N4CC(O)C4)ccc3F)cn2c1] | ¹H NMR (400 MHz, CD₃OD) δ 9.49 (s, 1H), 8.70 (d, J = 2.6 Hz, 1H), 8.47 (s, 1H), 8.29 (d, J = 2.6 Hz, 1H), 7.86 (dd, J = 8.8, 2.6 Hz, 1H), 7.59 (d, J = 8.8 Hz, 1H), 5.13-4.94 (m, 1H), 2.72 (s, 3H), 1.35 (d, J = 6.2 Hz, 6H). LCMS m/z = 429 (M + 1); RT = 0.76, Method 2. |
| 289 | 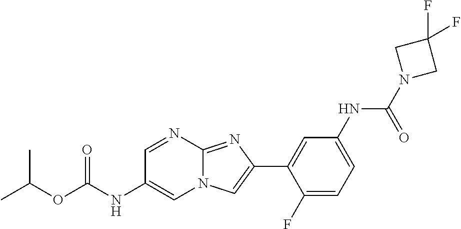 [SMILES: CC(C)OC(=O)Nc1cnc2nc(-c3cc(NC(=O)N4CC(F)(F)C4)ccc3F)cn2c1] | ¹H NMR (400 MHz, CD₃OD) δ 9.45 (s, 1H), 8.65 (d, J = 2.6 Hz, 1H), 8.35 (d, J = 3.1 Hz, 1H), 8.08 (dd, J = 6.6, 2.7 Hz, 1H), 7.56 (ddd, J = 8.9, 4.4, 2.8 Hz, 1H), 7.25 (dd, J = 10.7, 9.0 Hz, 1H), 5.04 (hept, J = 6.3 Hz, 1H), 4.44 (t, J = 12.3 Hz, 4H), 1.35 (d, J = 6.2 Hz, 6H). LCMS m/z = 449 (M + 1); RT = 0.9, Method 2. |
| 290 | 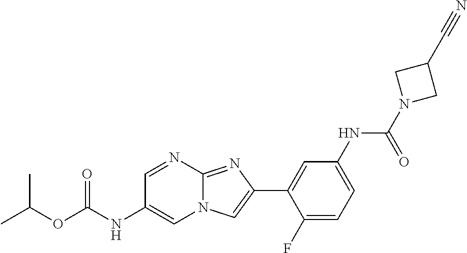 [SMILES: CC(C)OC(=O)Nc1cnc2nc(-c3cc(NC(=O)N4CC(C#N)C4)ccc3F)cn2c1] | ¹H NMR (400 MHz, CD₃OD) δ 9.46 (s, 1H), 8.66 (d, J = 2.5 Hz, 1H), 8.35 (d, J = 3.0 Hz, 1H), 8.06 (dd, J = 6.5, 2.7 Hz, 1H), 7.60-7.49 (m, 1H), 7.30-7.17 (m, 1H), 5.04 (hept, J = 6.3 Hz, 1H), 4.40 (t, J = 8.7 Hz, 2H), 4.25 (dd, J = 8.3, 6.0 Hz, 2H), 3.73 (s, 1H), 1.35 (d, J = 6.2 Hz, 6H). LCMS m/z = 438.1 (M + 1); RT = 0.83, Method 2. |
| 291 | 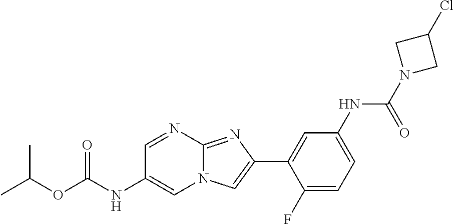 [SMILES: CC(C)OC(=O)Nc1cnc2nc(-c3cc(NC(=O)N4CC(Cl)C4)ccc3F)cn2c1] | LCMS m/z = 447.2 (M + 1); RT = 1.52, Method 3. |
| 292 | 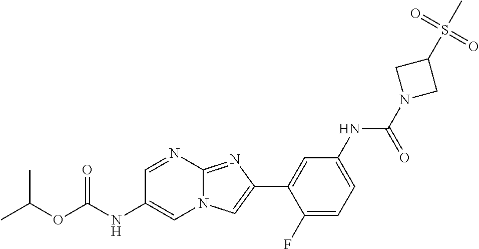 [SMILES: CC(C)OC(=O)Nc1cnc2nc(-c3cc(NC(=O)N4CC(S(C)(=O)=O)C4)ccc3F)cn2c1] | LCMS m/z = 491 (M + 1); RT = 0.79, Method 2. |
| 293 | 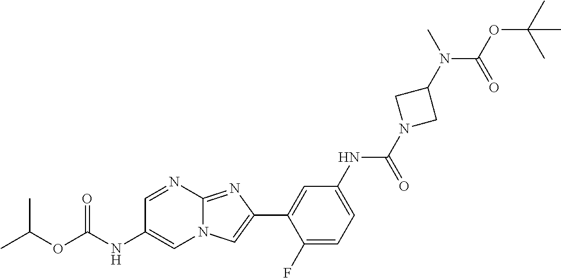 [SMILES: CC(C)OC(=O)Nc1cnc2nc(-c3cc(NC(=O)N4CC(N(C)C(=O)OC(C)(C)C)C4)ccc3F)cn2c1] | LCMS m/z = 542.1 (M + 1); RT = 0.99, Method 2. |
| 294 | 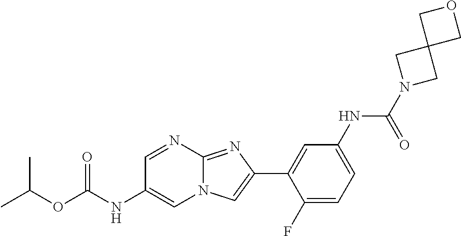 [SMILES: CC(C)OC(=O)Nc1cnc2nc(-c3cc(NC(=O)N4CC5(COC5)C4)ccc3F)cn2c1] | LCMS m/z = 455 (M + 1); RT = 0.8, Method 2. |
| 295 | 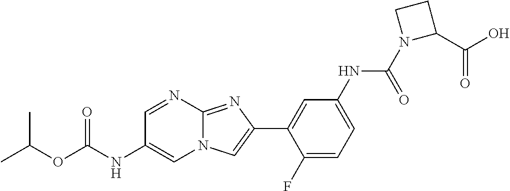 | LCMS m/z = 000 (M + 1); RT = 0.00, Method X. LIANGFA3-001-EXP090-007-1 |
| 296 | 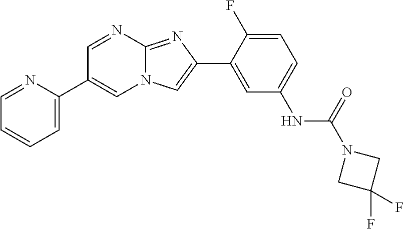 [SMILES: O=C(Nc1ccc(F)c(-c2cn3cc(-c4ccccn4)cnc3n2)c1)N1CC(F)(F)C1] | ¹H NMR (400 MHz, DMSO-d₆) δ 9.71 (s, 1H), 9.28 (s, 1H), 9.11 (s, 1H), 8.73 (d, J = 4.27 Hz, 1H), 8.30-8.45 (m, 2H), 8.08 (d, J = 7.78 Hz, 1H), 7.98 (td, J = 7.72, 1.63 Hz, 1H), 7.60-7.72 (m, 1H), 7.45 (dd, J = 7.15, 5.14 Hz, 1H), 7.27 (dd, J = 11.04, 9.03 Hz, 1H), 4.41 (t, J = 12.80 Hz, 4H). LCMS m/z = 434 (M + 1); RT = 2.93, Method 1. |
| 297 | 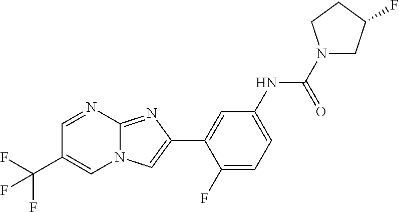 [SMILES: O=C(Nc1ccc(F)c(-c2cn3cc(C(F)(F)F)cnc3n2)c1)N1CC[C@H](F)C1] | ¹H NMR (400 MHz, DMSO-d₆) δ 9.67 (d, J = 1.00 Hz, 1H), 8.91 (d, J = 2.26 Hz, 1H), 8.53 (s, 1H), 8.44 (dd, J = 6.78, 2.51 Hz, 1H), 8.38 (d, J = 4.02 Hz, 1H), 7.65-7.74 (m, 1H), 7.24 (dd, J = 10.92, 9.16 Hz, 1H), 5.32-5.45 (m, 1H), 3.74-3.77 (m, 2H), 3.44-3.53 (m, 2H), 2.05-2.22 (m, 2H). LCMS m/z = 412.2 (M + 1); RT = 2.97, Method 1. |
| 298 | 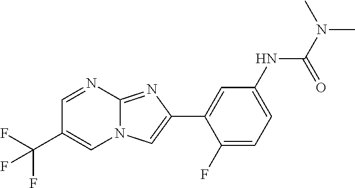 [SMILES: CN(C)C(=O)Nc1ccc(F)c(-c2cn3cc(C(F)(F)F)cnc3n2)c1] | ¹H NMR (400 MHz, DMSO-d₆) δ 9.66 (s, 1H), 8.90 (d, J = 2.26 Hz, 1H), 8.54 (s, 1H), 8.33-8.46 (m, 2H), 7.64 (dt, J = 7.91, 4.08 Hz, 1H), 7.22 (dd, J = 10.79, 9.29 Hz, 1H), 2.96 (s, 6H). LCMS m/z = 368 (M + 1); RT = 0.86, Method 4. |
| 299 | 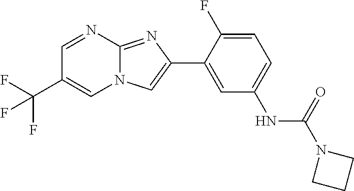 [SMILES: O=C(Nc1ccc(F)c(-c2cn3cc(C(F)(F)F)cnc3n2)c1)N1CCC1] | ¹H NMR (400 MHz, DMSO-d₆) δ 9.66 (s, 1H), 8.90 (d, J = 2.26 Hz, 1H), 8.62 (s, 1H), 8.33-8.44 (m, 2H), 7.70 (dt, J = 7.47, 4.30 Hz, 1H), 7.24 (dd, J = 10.92, 9.16 Hz, 1H), 3.98 (t, J = 7.65 Hz, 4H), 2.20 (quin, J = 7.53 Hz, 2H). LCMS m/z = 380 (M + 1); RT = 0.88, Method 4. |
| 300 | 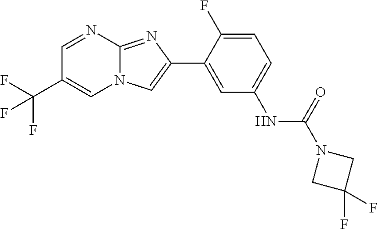 [SMILES: O=C(Nc1ccc(F)c(-c2cn3cc(C(F)(F)F)cnc3n2)c1)N1CC(F)(F)C1] | LCMS m/z = 416 (M + 1); RT = 0.98, Method 4. |
| 301 | 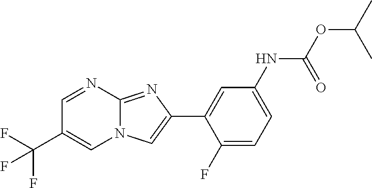 [SMILES: CC(C)OC(=O)Nc1ccc(F)c(-c2cn3cc(C(F)(F)F)cnc3n2)c1] | ¹H NMR (400 MHZ, DMSO-d₆) δ 9.75 (s, 1H), 9.64 (s, 1H), 8.90 (d, J = 4.0 Hz, 1H), 8.45 (d, J1 = 4.0 Hz, 1H), 8.34 (t, J = 8.0 Hz, 1H), 7.49 (t, J = 8.0 Hz, 1H), 7.29 (t, J = 8.0 Hz, 1H), 4.88-4.96 (m, 1H), 1.26 (s, 6H). LCMS m/z = 383 (M + 1); RT = 1.09, Method 4. |
| 302 | 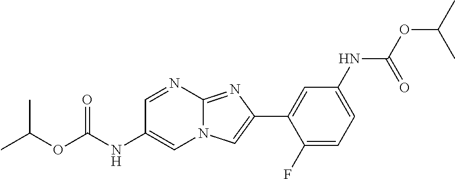 [SMILES: CC(C)OC(=O)Nc1ccc(F)c(-c2cn3cc(NC(=O)OC(C)C)cnc3n2)c1] | LCMS m/z = 416 (M + 1); RT = 1.02, Method 2. |
| 303 | 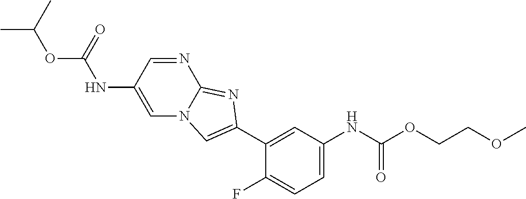 [SMILES: COCCOC(=O)Nc1ccc(F)c(-c2cn3cc(NC(=O)OC(C)C)cnc3n2)c1] | LCMS m/z = 432 (M + 1); RT = 1.1, Method 2. |
| 304 | 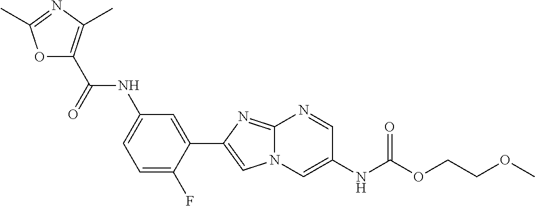 | ¹H NMR (400 MHz, CD₃OD) δ 9.59 (s, 1H), 8.80 (d, J = 2.5 Hz, 1H), 8.49 (d, J = 2.4 Hz, 1H), 8.38 (d, J = 3.9 Hz, 1H), 7.75 (dd, J = 7.2, 4.5 Hz, 1H), 7.39 (s, 1H), 4.48-4.32 (m, 2H), 3.78-3.62 (m, 2H), 3.41 (s, 3H), 2.57 (s, 3H), 2.49 (s, 3H). LCMS m/z = 469.4 (M + 1); RT = 1.46, Method 3. |
| 305 | 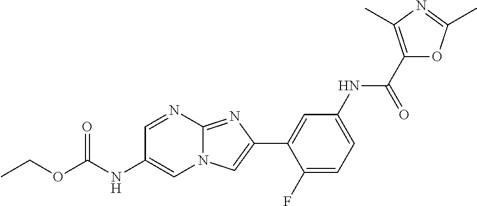 [SMILES: CCOC(=O)Nc1cnc2nc(-c3cc(NC(=O)c4oc(C)nc4C)ccc3F)cn2c1] | ¹H NMR (400 MHz, DMSO-d₆) δ 10.32 (s, 1H), 10.05 (s, 1H), 9.28 (s, 1H), 8.69 (dd, J = 2.8, 6.9 Hz, 1H), 8.50 (d, J = 2.7 Hz, 1H), 8.39 (d, J = 4.2, 1H), 7.77 (ddd, J = 2.8, 4.4, 8.9 Hz, 1H), 7.30 (dd, J = 9.0, 10.9 Hz, 1H), 4.19 (q, J = 7.1 Hz, 2H), 2.50 (s, 3H). 2.40 (s, 3H), 1.28 (t, J = 7.1 Hz, 3H). LCMS m/z = 439.4 (M + 1); RT = 1.52, Method 3. |
| 306 | 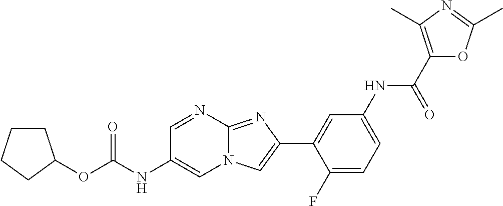 [SMILES: Cc1nc(C)c(C(=O)Nc2ccc(F)c(-c3cn4cc(NC(=O)OC5CCCC5)cnc4n3)c2)o1] | LCMS m/z = 479.4 (M + 1); RT = 1.72, Method 3. |
| 307 | 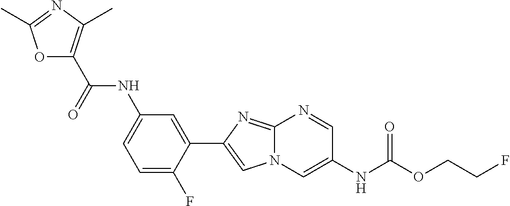 [SMILES: Cc1nc(C)c(C(=O)Nc2ccc(F)c(-c3cn4cc(NC(=O)OCCF)cnc4n3)c2)o1] | ¹H NMR (400 MHz, DMSO-d₆) δ 10.37- 10.21 (m, 2H), 9.29 (s, 1H), 8.70 (dd, J = 2.8, 6.9, 1H), 8.63 (dd, J = 2.8, 6.9, 0H), 8.52 (d, J = 2.7, 1H), 8.40 (d, J = 4.2, 1H), 8.26 (d, J = 2.7, 0H), 8.13 (dd, J = 3.5, 6.9, 0H), 7.80-7.70 (m, 1H), 7.30 (dt, J = 10.0, 20.1, 1H), 4.83- 4.58 (m, 2H), 4.50-4.28 (m, 2H), 2.50 (s, 3H), 2.40 (s, 3H). LCMS m/z = 457.4 (M + 1); RT = 1.46, Method 3. |
| 308 | 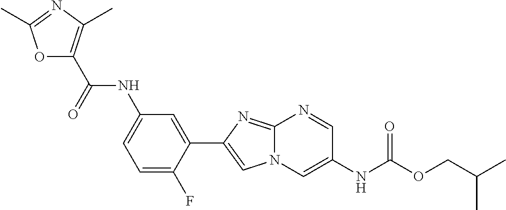 [SMILES: Cc1nc(C)c(C(=O)Nc2ccc(F)c(-c3cn4cc(NC(=O)OCC(C)C)cnc4n3)c2)o1] | ¹H NMR (400 MHz, DMSO-d₆) δ 10.31 (d, J = 10.8, 1H), 10.06 (s, 1H), 9.29 (s, 1H), 8.69 (dd, J = 2.8, 6.9, 1H), 8.52 (d, J = 2.7, 1H), 8.39 (d, J = 4.2, 1H), 7.77 (ddd, J = 2.8, 4.4, 8.9, 1H), 7.29 (dt, J = 11.3, 22.5, 1H), 3.94 (d, J = 6.6, 2H), 2.50 (s, 3H), 2.40 (s, 3H), 1.96 (dp, J = 6.7, 13.3, 1H), 0.95 (d, J = 6.7, 6H). LCMS m/z = 467.4 (M + 1); RT = 1.72, Method 3. |
| 309 | 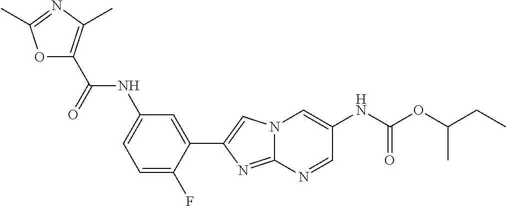 [SMILES: CCC(C)OC(=O)Nc1cnc2nc(-c3cc(NC(=O)c4oc(C)nc4C)ccc3F)cn2c1] | ¹H NMR (400 MHz, DMSO-d₆) δ 10.31 (d, J = 10.8, 1H), 10.06 (s, 1H), 9.29 (s, 1H), 8.69 (dd, J = 2.8, 6.9, 1H), 8.52 (d, J = 2.7, 1H), 8.39 (d, J = 4.2, 1H), 7.77 (ddd, J = 2.8, 4.4, 8.9, 1H), 7.29 (dt, J = 11.3, 22.5, 1H), 3.94 (d, J = 6.6, 2H), 2.50 (s, 3H), 2.40 (s, 3H), 1.96 (dp, J = 6.7, 13.3, 1H), 0.95 (d, J = 6.7, 6H). LCMS m/z = 467.4 (M + 1); RT = 1.68, Method 3. |
| 310 | 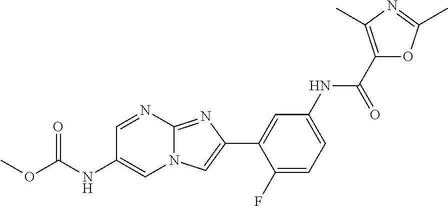 | ¹H NMR (400 MHz, DMSO-d₆) δ 10.32 (s, 1H), 10.07 (s, 1H), 9.27 (s, 1H), 8.69 (dd, J = 2.8, 6.9, 1H), 8.51 (d, J = 2.7, 1H), 8.40 (d, J = 4.2, 1H), 7.77 (ddd, J = 2.9, 4.4, 8.9, 1H), 7.31 (dd, J = 8.9, 10.9, 1H), 3.74 (s, 3H), 2.50 (s, 3H), 2.40 (s, 3H). LCMS m/z = 425.3 (M + 1); RT = 1.44, Method 3. |
| 311 | 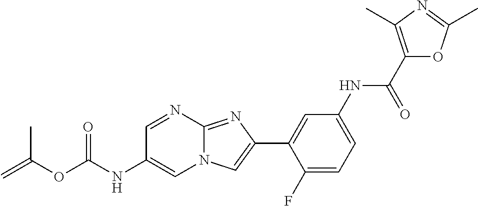 [SMILES: C=C(C)OC(=O)Nc1cnc2nc(-c3cc(NC(=O)c4oc(C)nc4C)ccc3F)cn2c1] | ¹H NMR (400 MHz, DMSO-d₆) δ 10.49- 10.25 (m, 2H), 9.30 (s, 1H), 8.70 (dd, J = 2.8, 6.9, 1H), 8.52 (d, J = 2.7, 1H), 8.40 (d, J = 4.2, 1H), 7.77 (ddd, J = 2.8, 4.4, 8.8, 1H), 7.30 (dt, J = 12.2, 24.3, 1H), 4.88-4.73 (m, 2H), 2.50 (s, 3H), 2.40 (s, 3H), 1.98 (s, 3H). LCMS m/z = 451.4 (M + 1); RT = 1.57, Method 3. |
| 312 | 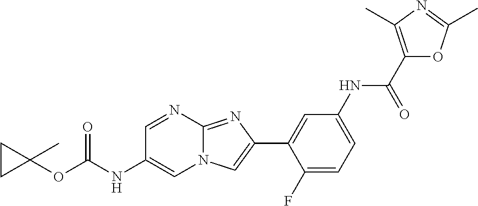 [SMILES: Cc1nc(C)c(C(=O)Nc2ccc(F)c(-c3cn4cc(NC(=O)OC5(C)CC5)cnc4n3)c2)o1] | ¹H NMR (400 MHz, DMSO-d₆) δ 10.33 (s, 1H), 10.04 (s, 1H), 9.34 (s, 1H), 8.67 (dd, J = 2.8, 6.9, 1H), 8.50 (d, J = 2.6, 1H), 8.40 (d, J = 4.1, 1H), 7.87- 7.67 (m, 1H), 7.32 (dd, J = 9.0, 10.9, 1H), 2.50 (s, 3H), 2.40 (s, 3H), 1.57 (s, 3H), 0.91 (s, 2H), 0.71 (t, J = 6.7, 2H). LCMS m/z = 465.4 (M + 1); RT = 1.61, Method 3. |
| 313 | 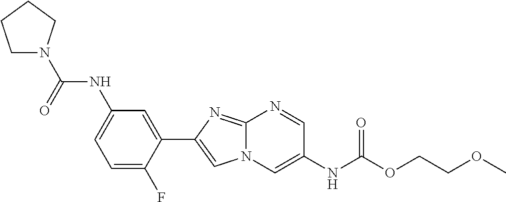 [SMILES: COCCOC(=O)Nc1cnc2nc(-c3cc(NC(=O)N4CCCC4)ccc3F)cn2c1] | ¹H NMR (400 MHz, CD₃OD) δ 9.54 (s, 1H), 8.80 (d, J = 2.6 Hz, 1H), 8.45 (d, J = 2.4 Hz, 1H), 8.07 (d, J = 6.7 Hz, 1H), 7.49 (s, 1H), 7.28 (d, J = 10.5 Hz, 1H), 4.44-4.32 (m, 2H), 3.75-3.63 (m, 2H), 3.56-3.47 (m, 4H), 3.41 (s, 3H), 2.01 (s, 4H). LCMS m/z = 443.2 (M + 1); RT = 1.33, Method 3. |
| 314 | 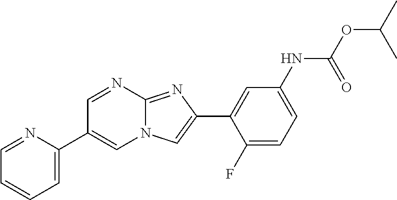 [SMILES: CC(C)OC(=O)Nc1ccc(F)c(-c2cn3cc(-c4ccccn4)cnc3n2)c1] | ¹H NMR (400 MHz, DMSO-d₆) δ 9.65- 9.75 (m, 2H), 9.27 (d, J = 2.51 Hz, 1H), 8.72 (d, J = 4.27 Hz, 1H), 8.43 (d, J = 4.02 Hz, 1H), 8.36 (d, J = 4.02 Hz, 1H), 8.07 (d, J = 8.03 Hz, 1H), 7.94-8.01 (m, 1H), 7.41-7.51 (m, 2H), 7.22-7.31 (m, 1H), 4.92 (dt, J = 12.36, 6.24 Hz, 1H), 1.27 (d, J = 6.27 Hz, 6H). LCMS m/z = 392.3 (M + 1); RT = 1.63, Method 3. |
| 315 | 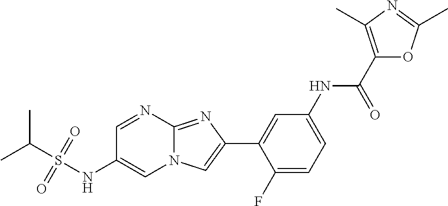 [SMILES: Cc1nc(C)c(C(=O)Nc2ccc(F)c(-c3cn4cc(NS(=O)(=O)C(C)C)cnc4n3)c2)o1] | ¹H NMR (400 MHz, DMSO-d₆) δ 10.34 (s, 1H), 10.06 (s, 1H), 8.98 (d, J = 2.7, 1H), 8.70 (dd, J = 2.7, 6.9, 1H), 8.49 (d, J = 2.7, 1H), 8.39 (d, J = 4.2, 1H), 7.86-7.71 (m, 1H), 7.33 (dd, J = 9.0, 10.9, 1H), 2.50 (s, 3H), 2.40 (s, 3H), 1.30 (d, J = 6.8, 6H). LCMS m/z = 473 (M + 1); RT = 0.84, Method 2. |
| 316 | 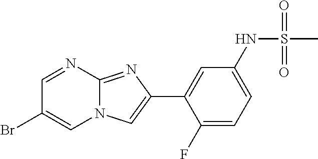 [SMILES: CS(=O)(=O)Nc1ccc(F)c(-c2cn3cc(Br)cnc3n2)c1] | ¹H NMR (400 MHz, DMSO-d₆) δ 9.86 (brs, 1H), 9.35 (d, J = 2.57 Hz, 1H), 8.65 (d, J = 2.57 Hz, 1H), 8.25 (d, J = 4.16 Hz, 1H), 8.15 (dd, J = 6.66, 2.75 Hz, 1H), 7.31-7.38 (m, 1H), 7.25 (ddd, J = 6.63, 4.43, 2.14 Hz, 1H), 2.99 (s, 3H). MS LCMS m/z = 386.8 (M + 1). LCMS m/z = 385.1 (M + 1), 387.1 (M + 3). RT = 1.39, Method 3. |
| 317 | 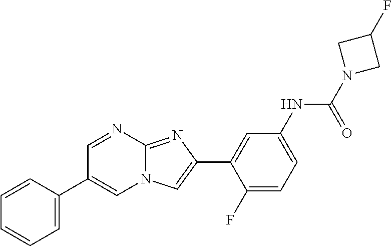 [SMILES: O=C(Nc1ccc(F)c(-c2cn3cc(-c4ccccc4)cnc3n2)c1)N1CC(F)C1] | ¹H NMR (400 MHz, DMSO-d₆) δ 9.34 (brs, 1H), 8.95 (brs, 1H), 8.83 (s, 1H), 8.39 (d, J = 4.27 Hz, 1H), 8.27 (d, J = 3.51 Hz, 1H), 7.78 (d, J = 7.28 Hz, 2H), 7.72-7.62 (m, 1H), 7.55 (t, J = 7.40 Hz, 2H), 7.47 (d, J = 7.03 Hz, 1H), 7.24 (t, J = 9.91 Hz, 1H), 5.56-5.27 (m, 1H), 4.42-4.22 (m, 2H), 4.12-3.91 (m, 2H). MS LCMS m/z = 406.0 (M + 1). LCMS m/z = 406.3 (M + 1); RT = 1.54. Method 3. |
| 318 | 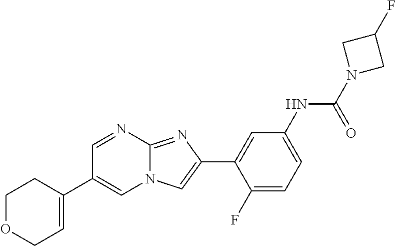 [SMILES: O=C(Nc1ccc(F)c(-c2cn3cc(C4=CCOCC4)cnc3n2)c1)N1CC(F)C1] | ¹H NMR (400 MHz, DMSO-d₆) δ 9.08 (s, 1H), 8.98 (s, 1H), 8.87 (s, 1H), 8.33 (dd, J = 6.78, 2.51 Hz, 1H), 8.27 (d, J = 3.76 Hz, 1H), 7.63 (dt, J = 7.34, 4.24 Hz, 1H), 7.26 (dd, J = 10.79, 9.03 Hz, 1H), 6.58 (brs. 1H), 5.28-5.52 (m, 1H), 4.30-4.41 (m, 2H), 4.28 (brs, 2H), 3.95- 4.12 (m, 2H), 3.87 (t, J = 5.40 Hz, 2H), 2.47 (m, 2H). LCMS m/z = 412.3 (M + 1); RT = 2.61, Method 1. |
| 319 | 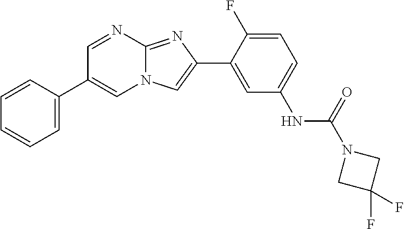 [SMILES: O=C(Nc1ccc(F)c(-c2cn3cc(-c4ccccc4)cnc3n2)c1)N1CC(F)(F)C1] | ¹H NMR (400 MHz, DMSO-d₆) δ 9.34 (s, 1H), 9.11 (s, 1H), 8.95 (s, 1H), 8.39 (dd, J = 6.65, 2.64 Hz, 1H), 8.28 (d, J = 4.02 Hz, 1H), 7.78 (d, J = 7.28 Hz, 2H), 7.67 (dt, J = 7.53, 4.27 Hz, 1H), 7.55 (t, J = 7.53 Hz, 2H), 7.42-7.50 (m, 1H), 7.26 (dd, J = 11.04, 9.03 Hz, 1H), 4.41 (t, J = 12.67 Hz, 1H). LCMS m/z = 424 (M + 1); RT = 1.07, Method 2. |
| 320 | 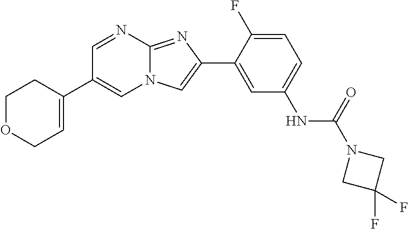 [SMILES: O=C(Nc1ccc(F)c(-c2cn3cc(C4=CCOCC4)cnc3n2)c1)N1CC(F)(F)C1] | ¹H NMR (400 MHz, DMSO-d₆) δ 9.08 (brs, 1H), 9.00 (br. s., 1H), 8.88 (brs, 1H), 8.35 (s, 1H), 8.22 (d, J = 3.51 Hz, 1H), 7.64 (m, 1H), 7.25 (t, J = 10.04 Hz, 1H), 6.54 (brs, 1H), 4.40 (t, J = 12.67 Hz, 3H), 4.27 (m, 2H), 3.87 (m, 2H), 2.46 (m, 2H). LCMS m/z = 430 (M + 1); RT = 0.91, Method 2. |
| 321 | 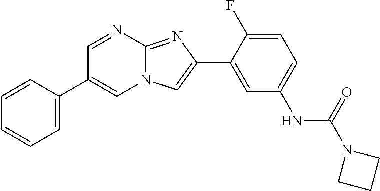 [SMILES: O=C(Nc1ccc(F)c(-c2cn3cc(-c4ccccc4)cnc3n2)c1)N1CCC1] | ¹H NMR (400 MHz, DMSO-d₆) δ 9.35 (s, 1H), 8.96 (s, 1H), 8.60 (s, 1H), 8.40-8.41 (m, 1H), 8.29 (d, J = 4.27 Hz, 1H), 8.27 (d, J = 4.0 Hz, 2H), 7.69- 7.80 (m, 1H), 7.55-7.58 (m, 2H), 7.48 (d, J = 7.2 Hz, 1H), 7.22-7.25 (m, 1H), 3.97-4.00 (m, 4H), 2.16-2.24 (m, 2H). LCMS m/z = 388.3 (M + 1); RT = 2.98, Method 1. |
| 322 | 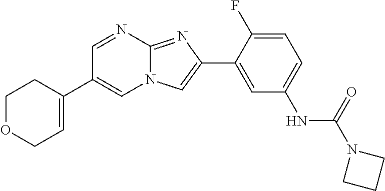 [SMILES: O=C(Nc1ccc(F)c(-c2cn3cc(C4=CCOCC4)cnc3n2)c1)N1CCC1] | ¹H NMR (400 MHz, DMSO-d₆) δ 9.01 (d, J = 2.26 Hz, 1H), 8.88 (d, J = 2.51 Hz, 1H), 8.58 (s, 1H), 8.36 (dd, J = 6.78, 2.76 Hz, 1H), 8.21 (d, J = 4.27 Hz, 1H), 7.64-7.70 (m, 1H), 7.21 (dd, J = 11.04, 9.03 Hz, 1H), 6.54 (brs, 1H), 4.28 (d, J = 2.51 Hz, 2H), 3.98 (t, J = 7.53 Hz, 4H), 3.88 (t, J = 5.40 Hz, 2H), 2.47 (brs, 2H), 2.16-2.23 (m, 2H), LCMS m/z = 394.1 (M + 1); RT = 0.83, Method 2. |
| 323 | 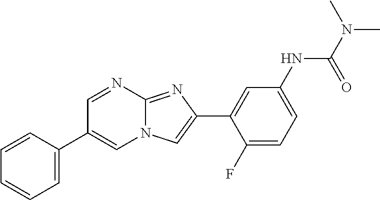 [SMILES: CN(C)C(=O)Nc1ccc(F)c(-c2cn3cc(-c4ccccc4)cnc3n2)c1] | ¹H NMR (400 MHz, DMSO-d₆) δ 9.34 (d, J = 2.51 Hz, 1H), 8.95 (d, J = 2.51 Hz, 1H), 8.51 (s, 1H), 8.39 (dd, J = 6.78, 2.76 Hz, 1H), 8.27 (d, J = 4.02 Hz, 1H), 7.78 (d, J = 7.53 Hz, 2H), 7.52-7.61 (m, 3H), 7.44-7.50 (m, 1H), 7.22 (dd, J = 11.04, 9.03 Hz, 1H) 2.95 (s, 6H). LCMS m/z = 376.3 (M + 1); RT = 1.5, Method 3. |
| 324 | 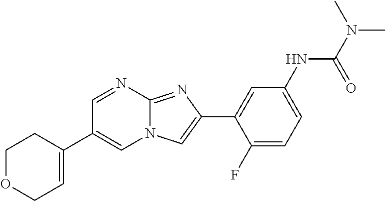 [SMILES: CN(C)C(=O)Nc1ccc(F)c(-c2cn3cc(C4=CCOCC4)cnc3n2)c1] | ¹H NMR (400 MHz, DMSO-d₆) δ 8.99 (s, 1H), 8.86 (s, 2H), 8.48 (s, 1H), 8.34 (m, 1H), 7.56 (m, 1H), 7.19 (t, J = 10.8 Hz, 1H), 6.52 (s, 1H), 4.26 (d, J = 2 Hz, 1H), 3.86 (t, J = 5.2 Hz, 1H), 2.94 (s, 6H), 2.46-2.5 (m, 2H). LCMS m/z = 382.2 (M + 1); RT = 1.31, Method 3. |
| 325 | 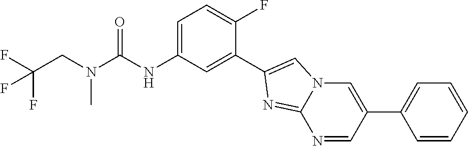 [SMILES: CN(CC(F)(F)F)C(=O)Nc1ccc(F)c(-c2cn3cc(-c4ccccc4)cnc3n2)c1] | ¹H NMR (400 MHz, DMSO-d₆) δ 9.35 (s, 1H), 8.96 (s, 1H), 8.78 (s, 1H), 8.44 (dd, J = 6.90, 2.64 Hz, 1H), 8.29 (d, J = 4.27 Hz, 1H), 7.79 (d, J = 7.28 Hz, 2H), 7.60-7.68 (m, 1H), 7.56 (t, J = 7.65 Hz, 2H), 7.41-7.50 (m, 1H), 7.26 (dd, J = 11.04, 9.03 Hz, 1H), 4.24 (q, J = 9.54 Hz, 2H), 3.14 (s, 3H). LCMS m/z = 444.1 (M + 1); RT = 1.1, Method 2. |
| 326 | 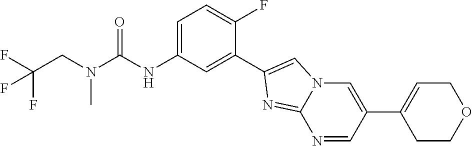 [SMILES: CN(CC(F)(F)F)C(=O)Nc1ccc(F)c(-c2cn3cc(C4=CCOCC4)cnc3n2)c1] | ¹H NMR (400 MHz, DMSO-d₆) δ 9.01 (s, 1H), 8.88 (s, 1H), 8.76 (s, 1H), 8.39 (dd, J = 6.78, 2.76 Hz, 1H), 8.22 (d, J = 4.27 Hz, 1H), 7.56-7.64 (m, 1H), 7.24 (dd, J = 11.04, 9.03 Hz, 1H), 6.54 (brs, 1H), 4.13-4.35 (m, 4H), 3.87 (t, J = 5.40 Hz, 2H), 3.13 (s, 3H), 2.47 (m, 2H). LCMS m/z = 450 (M + 1); RT = 0.96, Method 2. |
| 327 | 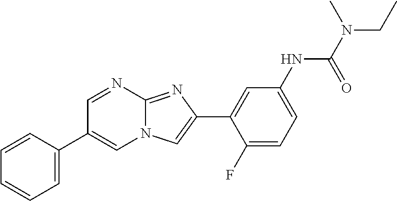 [SMILES: CCN(C)C(=O)Nc1ccc(F)c(-c2cn3cc(-c4ccccc4)cnc3n2)c1] | ¹H NMR (400 MHz, DMSO-d₆) δ 9.34 (d, J = 2.51 Hz, 1H), 8.95 (d, J = 2.51 Hz, 1H), 8.46 (s, 1H), 8.40 (dd, J = 6.90, 2.64 Hz, 1H), 8.27 (d, J = 4.02 Hz, 1H), 7.79 (d, J = 7.28 Hz, 2H), 7.57-7.66 (m, 1H), 7.52-7.56 (m, 2H), 7.42-7.51 (m, 1H), 7.21 (dd, J = 11.04, 9.03 Hz, 1H), 3.26-3.30 (m, 2H), 2.95 (s, 3H), 1.09 (t, J = 7.03 Hz, 3H). LCMS m/z = 390.3 (M + 1); RT = 3.04, Method 1. |
| 328 | 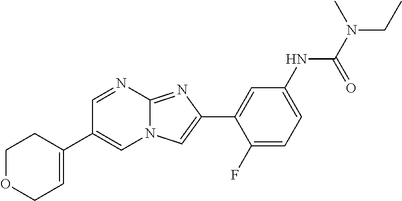 [SMILES: CCN(C)C(=O)Nc1ccc(F)c(-c2cn3cc(C4=CCOCC4)cnc3n2)c1] | ¹H NMR (400 MHz, DMSO-d₆) δ 9.00 (s, 1H), 8.87 (s, 1H), 8.43 (s, 1H), 8.37 (s, 1H), 8.20 (d, J = 4.27 Hz, 1H), 7.55- 7.64 (m, 1H), 7.19 (dd, J = 11.04, 9.03 Hz, 1H), 6.53 (br. s., 1H), 4.27 (d, J = 2.26 Hz, 2H), 3.87 (t, J = 5.40 Hz, 2H), 2.94 (s, 3H), 2.47 (br. s., 2H), 1.08 (t, J = 7.03 Hz, 3H). LCMS m/z = 396.2 (M + 1); RT = 1.41, Method 3. |
| 329 | 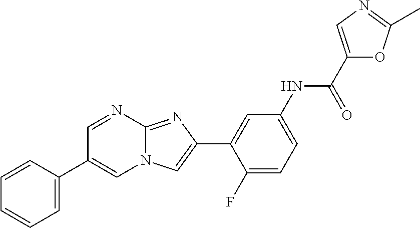 [SMILES: Cc1ncc(C(=O)Nc2ccc(F)c(-c3cn4cc(-c5ccccc5)cnc4n3)c2)o1] | ¹H NMR (400 MHz, DMSO-d₆) δ 10.51 (s, 1H), 9.35 (s, 1H), 8.97 (s, 1H), 8.65 (dd, J = 6.78, 2.51 Hz, 1H), 8.31 (d, J = 4.27 Hz, 1H), 7.84-7.98 (m, 2H), 7.79 (d, J = 7.53 Hz, 2H), 7.56 (t, J = 7.53 Hz, 2H), 7.42-7.51 (m, 1H), 7.30-7.41 (m, 1H), 2.54 (s, 3H). LCMS m/z = 414 (M + 1); RT = 1.02, Method 2. |
| 330 | 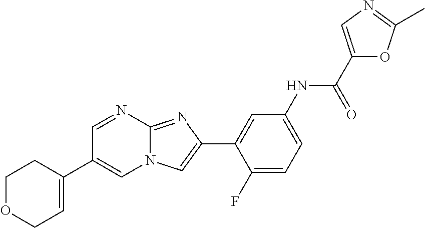 [SMILES: Cc1ncc(C(=O)Nc2ccc(F)c(-c3cn4cc(C5=CCOCC5)cnc4n3)c2)o1] | ¹H NMR (400 MHz, DMSO-d₆) δ 10.48 (s, 1H), 9.01 (s, 1H), 8.88 (s, 1H), 8.62 (dd, J = 6.78, 2.76 Hz, 1H), 8.25 (d, J = 4.02 Hz, 1H), 7.89 (s, 1H), 7.85 (dt, J = 7.84, 3.98 Hz, 1H), 7.34 (dd, J = 10.67, 9.16 Hz, 1H), 6.54 (br. s., 1H), 4.27 (d, J = 2.01 Hz, 2H), 3.87 (t, J = 5.40 Hz, 2H), 2.54 (s, 3H), 2.47 (m, 2H). LCMS m/z = 420 (M + 1); RT = 0.86, Method 2. |
| 331 | 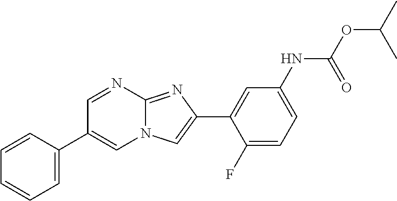 [SMILES: CC(C)OC(=O)Nc1ccc(F)c(-c2cn3cc(-c4ccccc4)cnc3n2)c1] | ¹H NMR (400 MHz, DMSO-d₆) δ 9.74 (brs, 1H), 9.36 (d, J = 2.51 Hz, 1H), 8.99 (d, J = 2.51 Hz, 1H), 8.45 (d, J = 4.27 Hz, 1H), 8.29 (d, J = 4 .02 Hz, 1H), 7.79 (d, J = 7.28 Hz, 2H), 7.56 (t, J = 7.53 Hz, 2H), 7.48-7.45 (m, 2H), 7.28 (dd, J = 10.92, 8.91 Hz, 1H), 4.92 (dt, J = 12.49, 6.18 Hz, 1H), 1.28-1.14 (m, 6H). LCMS m/z = 391 (M + 1); RT = 1.79, Method 3. |
| 332 | 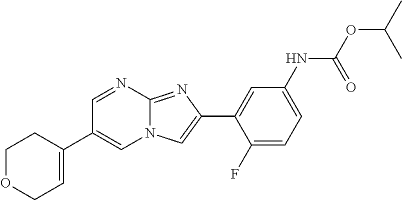 [SMILES: CC(C)OC(=O)Nc1ccc(F)c(-c2cn3cc(C4=CCOCC4)cnc3n2)c1] | LCMS m/z = 397.1 (M + 1); RT = 0.96, Method 2. |
| 333 | 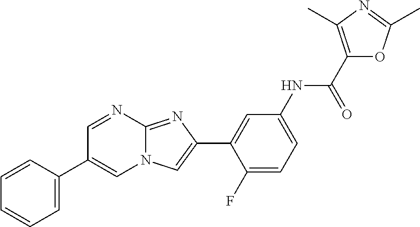 [SMILES: Cc1nc(C)c(C(=O)Nc2ccc(F)c(-c3cn4cc(-c5ccccc5)cnc4n3)c2)o1] | ¹H NMR (400 MHz, DMSO-d₆) δ 10.34 (s, 1H), 9.36 (d, J = 2.51 Hz, 1H), 8.97 (d, J = 2.51 Hz, 1H), 8.77 (dd, J = 6.90, 2.63 Hz, 1H), 8.31 (d, J = 4.27 Hz, 1H), 7.72-7.87 (m, 3H), 7.53-7.60 (m, 2H), 7.44-7.51 (m, 1H), 7.34 (dd, J = 10.79, 9.03 Hz, 1H), 2.52 (s, 3H), 2.42 (s, 3H). LCMS m/z = 428 (M + 1); RT = 1, Method 2. |
| 334 | 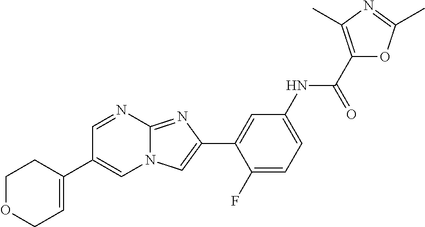 [SMILES: Cc1nc(C)c(C(=O)Nc2ccc(F)c(-c3cn4cc(C5=CCOCC5)cnc4n3)c2)o1] | ¹H NMR (400 MHz, DMSO-d₆) δ 10.33 (s, 1H), 9.01 (d, J = 2.6 Hz, 1H), 8.89 (d, J = 2.6 Hz, 1H), 8.72 (dd, J = 7.0, 2.7 Hz, 1H), 8.24 (d, J = 4.1 Hz, 1H), 7.78 (ddd, J = 8.8, 4.5, 2.8 Hz, 1H), 7.31 (dd, J = 11.0, 9.0 Hz, 1H), 6.60- 6.48 (m, 1H), 4.26 (d, J = 2.8 Hz, 2H), 3.87 (t, J = 5.4 Hz, 2H), 2.50 (s, 3H), 2.46 (d, J = 1.8 Hz, 2H), 2.40 (s, 3H). LCMS m/z = 434 (M + 1); RT = 0.85, Method 2. |
| 335 | 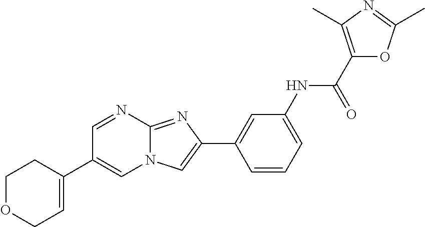 [SMILES: Cc1nc(C)c(C(=O)Nc2cccc(-c3cn4cc(C5=CCOCC5)cnc4n3)c2)o1] | ¹H NMR (400 MHz, DMSO-d₆) δ 10.24 (s, 1H), 8.95 (d, J = 4.0 Hz, 1H), 8.84 (d, J = 4.0 Hz, 1H), 8.47 (s, 1H), 8.26 (s, 1H), 7.73 (t, J = 8.0 Hz, 2H), 7.43 (t, J = 8.0 Hz, 1H), 6.54 (s, 1H), 4.27 (t, J = 8.0 Hz, 2H), 3.88 (t, J = 8.0 Hz, 2H), 2.50 (s, 3H), 2.40 (s, 3H). LCMS m/z = 416 (M + 1); RT = 0.73, Method 2. |
| 336 | 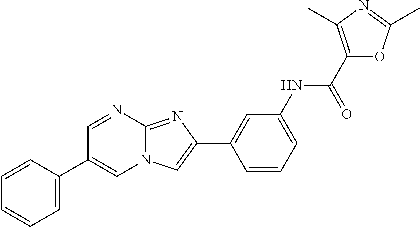 [SMILES: Cc1nc(C)c(C(=O)Nc2cccc(-c3cn4cc(-c5ccccc5)cnc4n3)c2)o1] | LCMS m/z = 410 (M + 1); RT = 0.89, Method 2. |
| 337 | 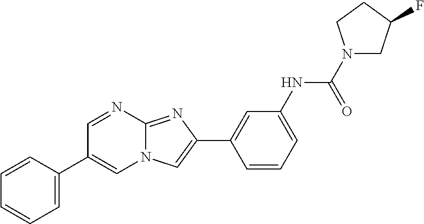 [SMILES: O=C(Nc1cccc(-c2cn3cc(-c4ccccc4)cnc3n2)c1)N1CC[C@@H](F)C1] | ¹H NMR (400 MHz, DMSO-d₆) δ 9.49 (d, J = 2.26 Hz, 1H), 9.15 (d, J = 2.01 Hz, 1H), 8.55 (s, 1H), 8.44 (s, 1H), 8.25 (s, 1H), 7.84 (d, J = 7.53 Hz, 2H), 7.55-7.68 (m, 4H), 7.52 (d, J = 7.28 Hz, 1H), 7.36-7.45 (m, 1H), 5.53-5.25 (m, 1H), 3.85-3.62 (m, 4H), 2.29-2.01 (m, 2H). LCMS m/z = 402.1 (M + 1); RT = 0.83, Method 2. |
| 338 | 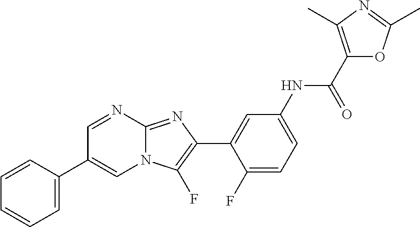 [SMILES: Cc1nc(C)c(C(=O)Nc2ccc(F)c(-c3nc4ncc(-c5ccccc5)cn4c3F)c2)o1] | ¹H NMR (400 MHz, DMSO-d₆) δ 10.38 (s, 1H), 9.00-9.12 (m, 2H), 8.49 (dd, J = 6.65, 2.64 Hz, 1H), 7.81-7.95 (m, 3H), 7.53-7.61 (m, 2H), 7.45-7.51 (m, 1H), 7.37 (t, J = 9.66 Hz, 1H), 2.41 (s, 3H), 2.39 (s, 3H). LCMS m/z = 446.2 (M + 1); RT = 2.02, Method 3. |
| 339 | 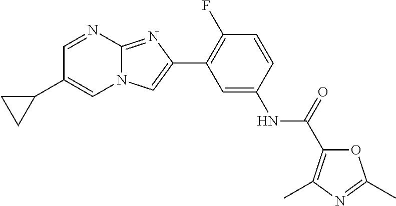 [SMILES: Cc1nc(C)c(C(=O)Nc2ccc(F)c(-c3cn4cc(C5CC5)cnc4n3)c2)o1] | ¹H NMR (400 MHz, DMSO-d₆) δ 10.39 (s, 1H), 8.97 (s, 1H), 8.72 (s, 1H), 8.63 (d, J = 2.8 Hz, 1H), 8.6 (m, 1H), 8.33 (s, 1H), 7.77-7.8 (m, 1H), 7.39-7.44 (m, 1H), 2.1-2.14 (m, 1H), 2.5 (s, 3H), 2.41 (s, 3H), 1.06-1.1 (m, 2H), 0.85-0.86 (m, 2H). LCMS m/z = 392.1 (M + 1); RT = 0.85, Method 4. |
| 340 | 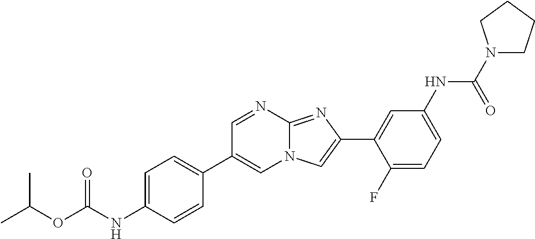 [SMILES: CC(C)OC(=O)Nc1ccc(-c2cnc3nc(-c4cc(NC(=O)N5CCCC5)ccc4F)cn3c2)cc1] | LCMS m/z = 503 (M + 1); RT = 0.99, Method 2. |
| 341 | 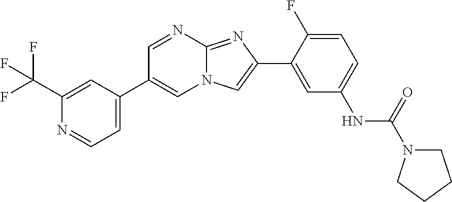 [SMILES: O=C(Nc1ccc(F)c(-c2cn3cc(-c4ccnc(C(F)(F)F)c4)cnc3n2)c1)N1CCCC1] | LCMS m/z = 470.9 (M + 1); RT = 0.95, Method 2. |
| 342 | 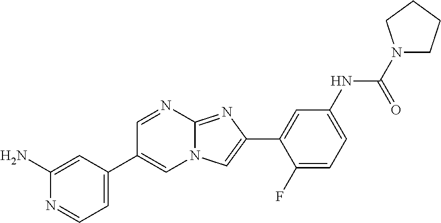 [SMILES: Nc1cc(-c2cnc3nc(-c4cc(NC(=O)N5CCCC5)ccc4F)cn3c2)ccn1] | LCMS m/z = 418 (M + 1); RT = 0.59, Method 2. |
| 343 | 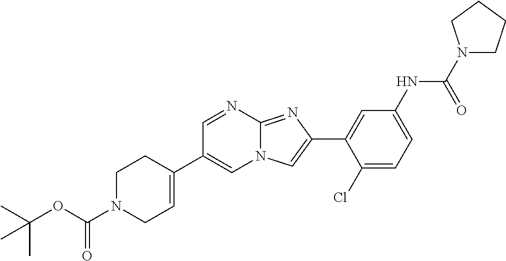 [SMILES: CC(C)(C)OC(=O)N1CC=C(c2cnc3nc(-c4cc(NC(=O)N5CCCC5)ccc4Cl)cn3c2)CC1] | ¹H NMR (400 MHz, CD₃OD) δ 8.83- 8.70 (m, 2H), 8.29 (s, 1H), 7.95 (d, J = 2.6 Hz, 1H), 7.57 (dd, J = 8.8, 2.7 Hz, 1H), 7.36 (d, J = 8.7 Hz, 1H), 6.33 (s, 1H), 4.10 (t, J = 3.6 Hz, 2H), 3.66 (t, J = 5.6 Hz, 2H), 3.56-3.40 (m, 4H), 2.52 (dq, J = 5.7, 3.7, 3.0 Hz, 2H), 2.06- 1.90 (m, 4H), 1.50 (s, 9H). LCMS m/z = 523.1 (M + 1); RT = 1.06, Method 2. |
| 344 | 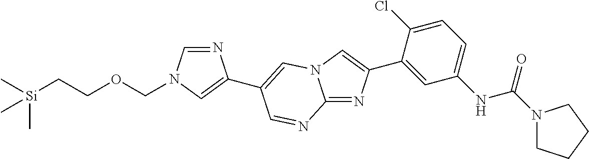 [SMILES: C[Si](C)(C)CCOCn1cnc(-c2cnc3nc(-c4cc(NC(=O)N5CCCC5)ccc4Cl)cn3c2)c1] | ¹H NMR (400 MHz, CD₃OD) δ 9.14 (d, J = 2.3 Hz, 1H), 8.99 (d, J = 2.3 Hz, 1H), 8.45 (s, 1H), 8.03-7.96 (m, 2H), 7.85 (s, 1H), 7.64-7.57 (m, 1H), 7.42 (d, J = 8.7 Hz, 1H), 5.45 (s, 2H), 3.63 (t, J = 8.0 Hz, 2H), 3.50 (d, J = 6.6 Hz, 4H), 1.98 (s, 4H), 0.95 (t, J = 8.0 Hz, 2H), 0.01 (s, 9H). LCMS m/z = 538.1 (M + 1); RT = 1.07, Method 2. |
| 345 | 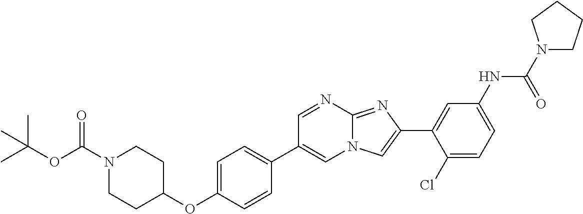 [SMILES: CC(C)(C)OC(=O)N1CCC(Oc2ccc(-c3cnc4nc(-c5cc(NC(=O)N6CCCC6)ccc5Cl)cn4c3)cc2)CC1] | ¹H NMR (400 MHz, CD₃OD) δ 8.97 (d, J = 2.5 Hz, 1H), 8.81 (d, J = 2.4 Hz, 1H), 8.35 (s, 1H), 7.98 (d, J = 2.6 Hz, 1H), 7.60 (d, J = 2.2 Hz, 2H), 7.58-7.53 (m, 1H), 7.36 (d, J = 8.9 Hz, 1H), 7.11- 7.03 (m, 2H), 4.61 (tt, J = 7.2, 3.5 Hz, 1H), 3.72 (ddd, J = 11.3, 7.1, 3.8 Hz, 2H), 3.53-3.43 (m, 4H), 3.40-3.33 (m, 2H), 2.01-1.90 (m, 6H), 1.68 (dtd, J = 15.3, 7.5, 3.7 Hz, 2H), 1.48 (s, 9H). LCMS m/z = 617.2 (M + 1); RT = 1.2, Method 2. |
| 346 | 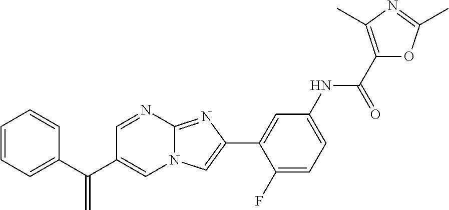 [SMILES: C=C(c1ccccc1)c1cnc2nc(-c3cc(NC(=O)c4oc(C)nc4C)ccc3F)cn2c1] | ¹H NMR (400 MHz, CD₃OD) δ 8.84 (d, J = 2.4 Hz, 1H), 8.56 (d, J = 2.5 Hz, 1H), 8.28 (dd, J = 6.7, 2.7 Hz, 1H), 8.23 (d, J = 3.9 Hz, 1H), 7.83 (ddd, J = 8.8, 4.4, 2.8 Hz, 1H), 7.46-7.38 (m, 5H), 7.24 (dd, J = 10.9, 8.9 Hz, 1H), 5.71 (s, 1H), 5.68 (s, 1H), 2.56 (s, 3H), 2.47 (s, 3H). LCMS m/z = 454.4 (M + 1); RT = 1.85, Method 3. |
| 347 | 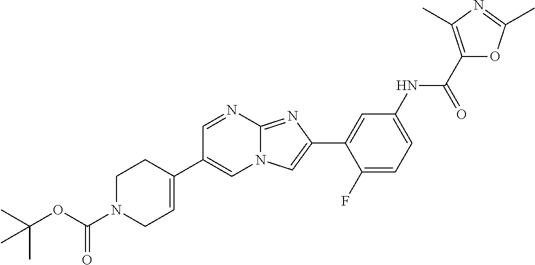 [SMILES: Cc1nc(C)c(C(=O)Nc2ccc(F)c(-c3cn4cc(C5=CCN(C(=O)OC(C)(C)C)CC5)cnc4n3)c2)o1] | ¹H NMR (400 MHz, CD₃OD) δ 8.85- 8.68 (m, 2H), 8.19 (dd, J = 6.7, 2.7 Hz, 1H), 8.10 (d, J = 3.7 Hz, 1H), 7.80 (ddd, J = 8.7, 4.5, 2.7 Hz, 1H), 7.18 (dd, J = 10.9, 8.9 Hz, 1H), 6.34 (s, 1H), 4.10 (s, 2H), 3.66 (t, J = 5.6 Hz, 2H), 2.54 (d, J = 8.8 Hz, 5H), 2.46 (s, 3H), 1.50 (s, 9H). LCMS m/z = 533.4 (M + 1); RT = 3.24, Method 1. |
| 348 | 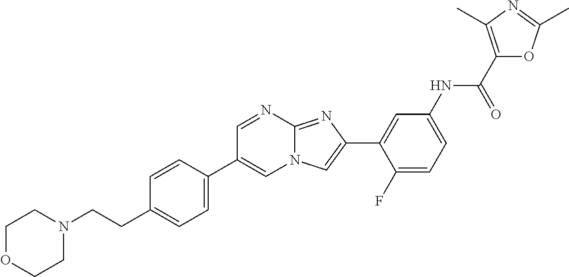 [SMILES: Cc1nc(C)c(C(=O)Nc2ccc(F)c(-c3cn4cc(-c5ccc(CCN6CCOCC6)cc5)cnc4n3)c2)o1] | ¹H NMR (400 MHz, DMSO-d₆) δ 10.35 (s, 1H), 9.32 (d, J = 2.6 Hz, 1H), 8.95 (d, J = 2.5 Hz, 1H), 8.76 (dd, J = 6.8, 2.8 Hz, 1H), 8.29 (d, J = 4.1 Hz, 1H), 7.81 (ddd, J = 8.8, 4.4, 2.8 Hz, 1H), 7.74-7.63 (m, 2H), 7.45-7.39 (m, 2H), 7.34 (dd, J = 11.0, 8.9 Hz, 1H), 3.58 (t, J = 4.6 Hz, 4H), 2.81 (dd, J = 8.9, 6.4 Hz, 2H), 2.55 (dd, J = 9.0, 6.4 Hz, 2H), 2.51 (s, 3H), 2.44 (t, J = 4.4 Hz, 4H), 2.41 (s, 3H). LCMS m/z = 541.2 (M + 1); RT = 0.67, Method 2. |
| 349 | 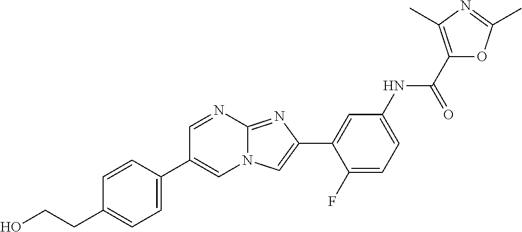 [SMILES: Cc1nc(C)c(C(=O)Nc2ccc(F)c(-c3cn4cc(-c5ccc(CCO)cc5)cnc4n3)c2)o1] | ¹H NMR (400 MHz, CD₃OD) δ 9.08 (d, J = 2.5 Hz, 1H), 8.87 (d, J = 2.4 Hz, 1H), 8.29-8.19 (m, 2H), 7.82 (ddd, J = 8.8, 4.4, 2.7 Hz, 1H), 7.68-7.61 (m, 2H), 7.40 (d, J = 7.8 Hz, 2H), 7.22 (dd, J = 10.9, 8.9 Hz, 1H), 3.80 (t, J = 6.9 Hz, 2H), 2.89 (t, J = 6.9 Hz, 2H), 2.56 (s, 3H), 2.46 (s, 3H). LCMS m/z = 472.4 (M + 1); RT = 1.55, Method 3. |
| 350 | 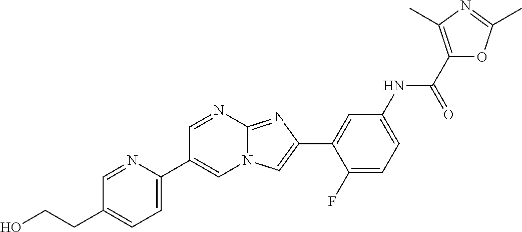 [SMILES: Cc1nc(C)c(C(=O)Nc2ccc(F)c(-c3cn4cc(-c5ccc(CCO)cn5)cnc4n3)c2)o1] | ¹H NMR (400 MHz, DMSO-d₆) δ 10.36 (s, 1H), 9.69 (d, J = 2.4 Hz, 1H), 9.28 (d, J = 2.4 Hz, 1H), 8.76 (dd, J = 7.0, 2.7 Hz, 1H), 8.59 (d, J = 2.3 Hz, 1H), 8.40 (d, J = 4.1 Hz, 1H), 8.00 (d, J = 8.1 Hz, 1H), 7.89-7.75 (m, 2H), 7.34 (dd, J = 11.0, 9.0 Hz, 1H), 4.76 (t, J = 5.1 Hz, 1H), 3.67 (td, J = 6.6, 5.1 Hz, 2H), 2.81 (t, J = 6.6 Hz, 2H), 2.51 (d, J = 1.1 Hz, 3H), 2.41 (s, 3H). LCMS m/z = 473.3 (M + 1); RT = 1.44, Method 3. |
| 351 | 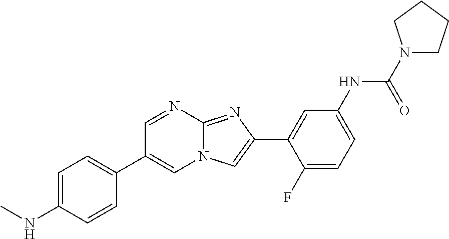 [SMILES: CNc1ccc(-c2cnc3nc(-c4cc(NC(=O)N5CCCC5)ccc4F)cn3c2)cc1] | ¹H NMR (400 MHz, CD₃OD) δ 8.95 (d, J = 2.4 Hz, 1H), 8.84 (d, J = 2.4 Hz, 1H), 8.19 (d, J = 3.8 Hz, 1H), 7.99 (dd, J = 6.6, 2.7 Hz, 1H), 7.58 (ddd, J = 8.8, 4.4, 2.8 Hz, 1H), 7.50 (d, J = 7.9 Hz, 2H), 7.15 (dd, J = 11.0, 8.9 Hz, 1H), 6.75 (d, J = 8.3 Hz, 2H), 3.53-3.47 (m, 4H), 2.82 (s, 3H), 1.99 (q, J = 3.8 Hz, 4H). LCMS m/z = 431.2 (M + 1); RT = 2.86, Method 1. |
| 352 | 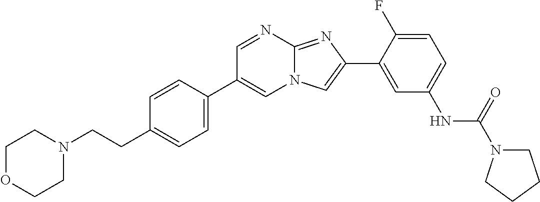 [SMILES: O=C(Nc1ccc(F)c(-c2cn3cc(-c4ccc(CCN5CCOCC5)cc4)cnc3n2)c1)N1CCCC1] | ¹H NMR (400 MHz, CD₃OD) δ 9.09 (d, J = 2.5 Hz, 1H), 8.88 (d, J = 2.5 Hz, 1H), 8.23 (d, J = 3.8 Hz, 1H), 8.01 (dd, J = 6.5, 2.8 Hz, 1H), 7.69-7.62 (m, 2H), 7.58 (ddd, J = 8.9, 4.4, 2.8 Hz, 1H), 7.44-7.37 (m, 2H), 7.15 (dd, J = 11.0, 8.9 Hz, 1H), 3.73 (t, J = 4.7 Hz, 4H), 3.54-3.45 (m, 4H), 2.94-2.85 (m, 2H), 2.69-2.61 (m, 2H), 2.57 (t, J = 4.6 Hz, 4H), 2.05-1.94 (m, 4H). LCMS m/z = 515.2 (M + 1); RT = 0.65, Method 2. |
| 353 | 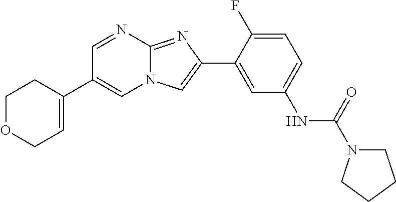 [SMILES: O=C(Nc1ccc(F)c(-c2cn3cc(C4=CCOCC4)cnc3n2)c1)N1CCCC1] | ¹H NMR (400 MHz, CD₃OD) δ 8.89- 8.76 (m, 2H), 8.17 (d, J = 3.8 Hz, 1H), 7.99 (dd, J = 6.6, 2.8 Hz, 1H), 7.58 (ddd, J = 9.0, 4.5, 2.8 Hz, 1H), 7.15 (dd, J = 11.0, 8.9 Hz, 1H), 6.50-6.40 (m, 1H), 4.35 (q, J = 2.8 Hz, 2H), 3.97 (t, J = 5.4 Hz, 2H), 3.55-3.43 (m, 4H), 2.55 (dq, J = 5.8, 3.0 Hz, 2H), 2.07- 1.88 (m, 4H). LCMS m/z = 408.1 (M + 1); RT = 0.79, Method 2. |
| 354 | 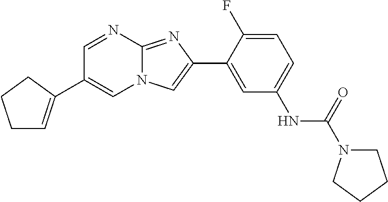 [SMILES: O=C(Nc1ccc(F)c(-c2cn3cc(C4=CCCC4)cnc3n2)c1)N1CCCC1] | ¹H NMR (400 MHz, CD₃OD) δ 8.90 (d, J = 2.4 Hz, 1H), 8.75 (d, J = 2.3 Hz, 1H), 8.16 (d, J = 3.8 Hz, 1H), 7.98 (dd, J = 6.6, 2.8 Hz, 1H), 7.58 (ddd, J = 8.9, 4.4, 2.8 Hz, 1H), 7.15 (dd, J = 11.0, 8.9 Hz, 1H), 6.51 (t, J = 2.2 Hz, 1H), 3.54- 3.44 (m, 4H), 2.76 (tt, J = 6.1, 2.1 Hz, 2H), 2.62 (td, J = 7.9, 7.5, 2.5 Hz, 2H), 2.14-2.05 (m, 2H), 2.03-1.95 (m, 4H). LCMS m/z = 392.3 (M + 1); RT = 1.61, Method 3. |
| 355 | 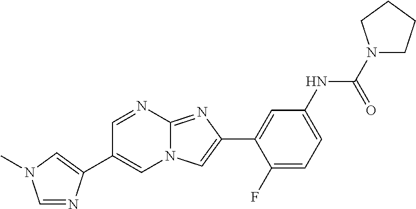 [SMILES: Cn1cnc(-c2cnc3nc(-c4cc(NC(=O)N5CCCC5)ccc4F)cn3c2)c1] | ¹H NMR (400 MHz, CD₃OD) δ 9.05 (d, J = 2.3 Hz, 1H), 8.93 (d, J = 2.3 Hz, 1H), 8.22 (d, J = 3.7 Hz, 1H), 7.99 (dd, J = 6.5, 2.7 Hz, 1H), 7.76 (d, J = 1.3 Hz, 1H), 7.65 (d, J = 1.3 Hz, 1H), 7.58 (ddd, J = 8.8, 4.5, 2.8 Hz, 1H), 7.15 (dd, J = 11.0, 8.9 Hz, 1H), 3.81 (s, 3H), 3.55-3.45 (m, 4H), 2.04-1.95 (m, 4H). LCMS m/z = 406.2 (M + 1); RT = 1.22, Method 3. |
| 356 | 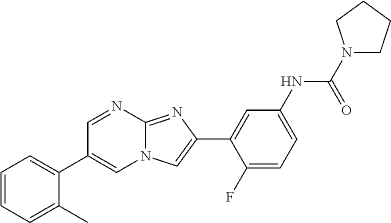 [SMILES: Cc1ccccc1-c1cnc2nc(-c3cc(NC(=O)N4CCCC4)ccc3F)cn2c1] | ¹H NMR (400 MHz, CD₃OD) δ 8.87 (d, J = 2.4 Hz, 1H), 8.59 (d, J = 2.3 Hz, 1H), 8.25 (d, J = 3.8 Hz, 1H), 8.03 (dd, J = 6.7, 2.8 Hz, 1H), 7.59 (ddd, J = 8.8, 4.5, 2.8 Hz, 1H), 7.42-7.29 (m, 4H), 7.16 (dd, J = 11.0, 9.0 Hz, 1H), 3.56- 3.43 (m, 4H), 2.37 (s, 3H), 2.08-1.93 (m, 4H). LCMS m/z = 416.3 (M + 1); RT = 1.65, Method 3. |
| 357 | 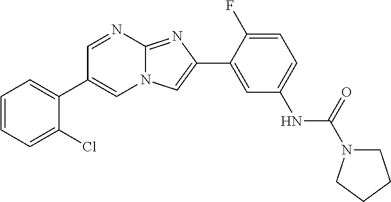 [SMILES: O=C(Nc1ccc(F)c(-c2cn3cc(-c4ccccc4Cl)cnc3n2)c1)N1CCCC1] | ¹H NMR (400 MHz, CD₃OD) δ 9.00 (d, J = 2.4 Hz, 1H), 8.69 (d, J = 2.4 Hz, 1H), 8.28 (d, J = 3.7 Hz, 1H), 8.04 (dd, J = 6.6, 2.7 Hz, 1H), 7.67-7.54 (m, 3H), 7.49 (dd, J = 5.8, 3.5 Hz, 2H), 7.17 (dd, J = 10.9, 9.0 Hz, 1H), 3.57-3.44 (m, 4H), 2.09-1.90 (m, 4H). LCMS m/z = 436.2 (M + 1); RT = 1.68, Method 3. |
| 358 | 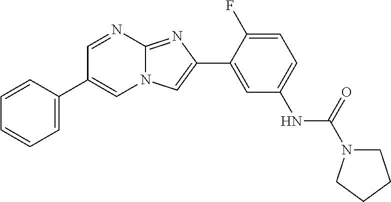 [SMILES: O=C(Nc1ccc(F)c(-c2cn3cc(-c4ccccc4)cnc3n2)c1)N1CCCC1] | ¹H NMR (400 MHz, DMSO-d₆) δ 9.35 (d, J = 2.5 Hz, 1H), 8.95 (d, J = 2.5 Hz, 1H), 8.43 (dd, J = 6.8, 2.8 Hz, 1H), 8.37 (s, 1H), 8.27 (d, J = 4.1 Hz, 1H), 7.83-7.75 (m, 2H), 7.68 (ddd, J = 8.9, 4.5, 2.8 Hz, 1H), 7.60-7.51 (m, 2H), 7.50-7.41 (m, 1H), 7.22 (dd, J = 11.1, 9.0 Hz, 1H), 3.40 (q, J = 5.0, 3.5 Hz, 4H), 1.92-1.80 (m, 4H). LCMS m/z = 402.3 (M + 1); RT = 3.05, Method 1. |
| 359 | 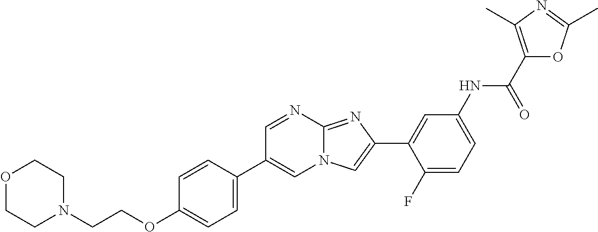 [SMILES: Cc1nc(C)c(C(=O)Nc2ccc(F)c(-c3cn4cc(-c5ccc(OCCN6CCOCC6)cc5)cnc4n3)c2)o1] | LCMS m/z = 557.4 (M + 1); RT = 1.44, Method 3. |
| 360 | 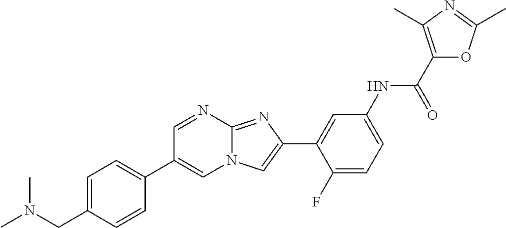 | ¹H NMR (400 MHz, CD₃OD) δ 9.32 (d, J = 2.4 Hz, 1H), 9.08 (d, J = 2.4 Hz, 1H), 8.41-8.30 (m, 2H), 7.96-7.88 (m, 2H), 7.77 (ddd, J = 8.8, 4.4, 2.7 Hz, 1H), 7.73-7.66 (m, 2H), 7.29 (dd, J = 10.8, 8.9 Hz, 1H), 4.41 (s, 2H), 2.92 (s, 6H), 2.55 (s, 3H), 2.46 (s, 3H). LCMS m/z = 485.4 (M + 1); RT = 1.37, Method 3. |
| 361 | 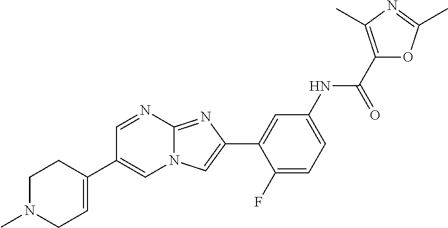 | ¹H NMR (400 MHz, CD₃OD) δ 9.03 (d, J = 2.5 Hz, 1H), 8.89 (d, J = 2.5 Hz, 1H), 8.35 (dd, J = 6.6, 2.7 Hz, 1H), 8.28 (d, J = 3.5 Hz, 1H), 7.81 (ddd, J = 8.9, 4.4, 2.8 Hz, 1H), 7.28 (dd, J = 10.9, 8.9 Hz, 1H), 6.45 (td, J = 3.5, 1.8 Hz, 1H), 4.13 (bs, 1H), 3.92 (bs, 1H), 3.79 (bs, 1H), 3.45 (bs, 1H), 3.06 (s, 3H), 2.96 (bs, 2H), 2.56 (s, 3H), 2.47 (s, 3H). LCMS m/z = 447 (M + 1); RT = 0.58, Method 2. |
| 362 | 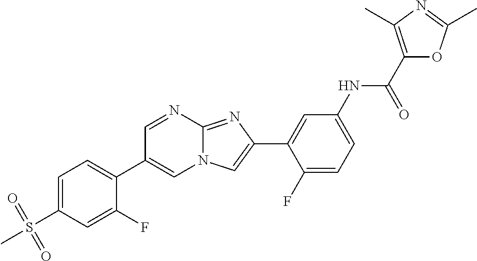 | ¹H NMR (400 MHz, CD₃OD) δ 9.29 (d, J = 2.4 Hz, 1H), 8.90 (t, J = 2.1 Hz, 1H), 8.40-8.33 (m, 2H), 8.02-7.92 (m, 3H), 7.85 (ddd, J = 8.9, 4.4, 2.7 Hz, 1H), 7.29 (dd, J = 10.9, 9.0 Hz, 1H), 3.23 (s, 3H), 2.57 (s, 3H), 2.48 (s, 3H). LCMS m/z = 524.2 (M + 1); RT = 1.63, Method 3. |
| 363 | 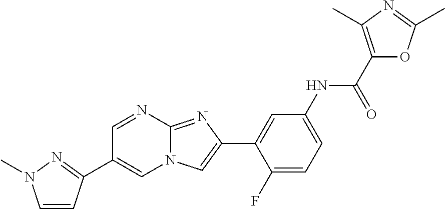 | ¹H NMR (400 MHz, CD₃OD) δ 9.36 (d, J = 2.4 Hz, 1H), 9.25 (d, J = 2.4 Hz, 1H), 8.40 (m, 1H), 8.35 (m, 1H), 7.80 (dd, J = 6.6, 2.7 Hz, 1H), 7.74 (d, J = 3.7 Hz, 1H), 7.35 (ddd, J = 8.8, 4.4, 2.8 Hz, 1H), 6.83 (d, J = 2.4 Hz, 1H), 4.01 (s, 3H), 2.57 (s, 3H), 2.48 (s, 3H). LCMS m/z = 432.3 (M + 1); RT = 2.8, Method 1. |
| 364 | 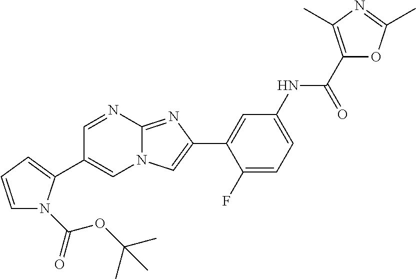 [SMILES: Cc1nc(C)c(C(=O)Nc2ccc(F)c(-c3cn4cc(-c5cccn5C(=O)OC(C)(C)C)cnc4n3)c2)o1] | ¹H NMR (400 MHz, CD₃OD) δ 8.91 (d, J = 2.0 Hz, 1H), 8.69 (d, J = 2.4 Hz, 1H), 8.31 (dt, J = 9.5, 3.1 Hz, 2H), 7.82 (ddd, J = 9.2, 4.6, 2.7 Hz, 1H), 7.50 (dd, J = 3.3, 1.6 Hz, 1H), 7.27 (dd, J = 10.8, 8.8 Hz, 1H), 6.48 (dd, J = 3.2, 1.9 Hz, 1H), 6.35 (t, J = 3.2 Hz, 1H), 2.56 (s, 3H), 2.47 (s, 3H), 1.46 (s, 9H). LCMS m/z = 517.1 (M + 1); RT = 1.14, Method 2. |
| 365 | 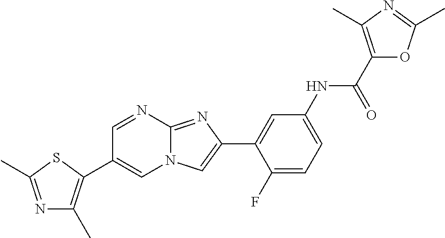 | LCMS m/z = 463.3 (M + 1); RT = 2.93, Method 1. |
| 366 | 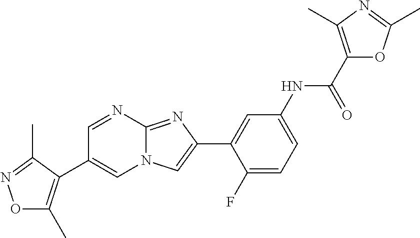 [SMILES: Cc1nc(C)c(C(=O)Nc2ccc(F)c(-c3cn4cc(-c5c(C)noc5C)cnc4n3)c2)o1] | ¹H NMR (400 MHz, CD₃OD) δ 9.07 (d, J = 2.3 Hz, 1H), 8.75 (d, J = 2.3 Hz, 1H), 8.37 (dd, J = 5.5, 3.0 Hz, 2H), 7.86-7.76 (m, 1H), 7.32 (dd, J = 11.0, 9.2 Hz, 1H), 2.56 (s, 3H), 2.52 (s, 3H), 2.48 (s, 3H), 2.35 (s, 3H). LCMS m/z = 447 (M + 1); RT = 0.88, Method 2. |
| 367 | 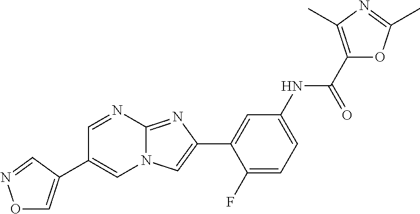 [SMILES: Cc1nc(C)c(C(=O)Nc2ccc(F)c(-c3cn4cc(-c5cnoc5)cnc4n3)c2)o1] | LCMS m/z = 419 (M + 1); RT = 0.78, Method 2. |
| 368 | 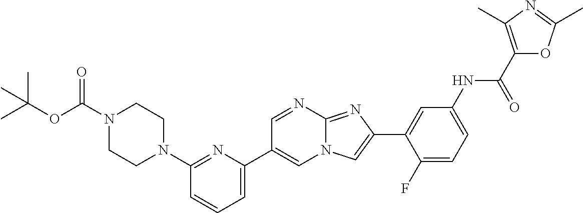 [SMILES: Cc1nc(C)c(C(=O)Nc2ccc(F)c(-c3cn4cc(-c5cccc(N6CCN(C(=O)OC(C)(C)C)CC6)n5)cnc4n3)c2)o1] | LCMS m/z = 613.4 (M + 1); RT = 2.11, Method 3. |
| 369 | 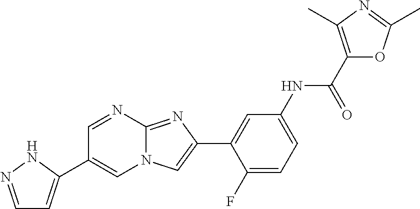 [SMILES: Cc1nc(C)c(C(=O)Nc2ccc(F)c(-c3cn4cc(-c5ccn[nH]5)cnc4n3)c2)o1] | ¹H NMR (400 MHz, CD₃OD) δ 9.45 (d, J = 2.3 Hz, 1H), 9.37 (d, J = 2.3 Hz, 1H), 8.46 (d, J = 2.7 Hz, 1H), 8.39 (dd, J = 6.7, 2.7 Hz, 1H), 7.84 (d, J = 2.4 Hz, 1H), 7.78 (ddd, J = 9.0, 4.5, 2.7 Hz, 1H), 7.37 (dd, J = 10.7, 9.0 Hz, 1H), 6.91 (d, J = 2.5 Hz, 1H), 2.56 (s, 3H), 2.48 (s, 3H). LCMS m/z = 418.2 (M + 1); RT = 1.41, Method 3. |
| 370 | 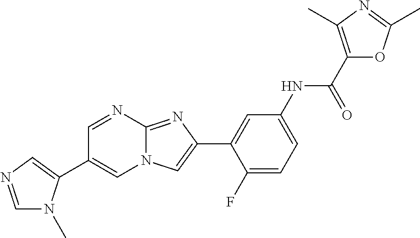 [SMILES: Cc1nc(C)c(C(=O)Nc2ccc(F)c(-c3cn4cc(-c5cncn5C)cnc4n3)c2)o1] | LCMS m/z = 432 (M + 1); RT = 0.59, Method 2. |
| 371 | 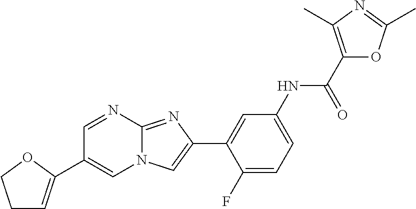 [SMILES: Cc1nc(C)c(C(=O)Nc2ccc(F)c(-c3cn4cc(C5=CCCO5)cnc4n3)c2)o1] | ¹H NMR (400 MHz, CD₃OD) δ 9.02 (d, J = 2.3 Hz, 1H), 8.72 (d, J = 2.3 Hz, 1H), 8.33 (d, J = 3.3 Hz, 2H), 7.82 (ddd, J = 8.8, 4.4, 2.7 Hz, 1H), 7.30 (dd, J = 10.8, 9.0 Hz, 1H), 4.01-3.83 (m, 1H), 2.53 (s, 3H), 2.49 (s, 3H), 2.16 (d, J = 13.2 Hz, 1H), 2.09-1.99 (m, 1H), 1.78-1.60 (m, 2H). LCMS m/z = 420 (M + 1); RT = 1.01, Method 2. |
| 372 | 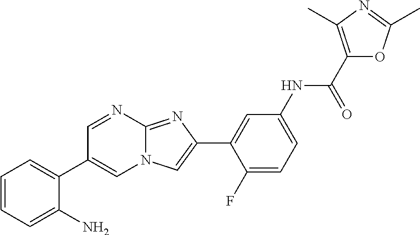 [SMILES: Cc1nc(C)c(C(=O)Nc2ccc(F)c(-c3cn4cc(-c5ccccc5N)cnc4n3)c2)o1] | LCMS m/z = 443.4 (M + 1); RT = 1.55, Method 3. |
| 373 | 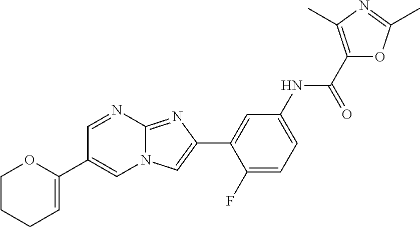 [SMILES: Cc1nc(C)c(C(=O)Nc2ccc(F)c(-c3cn4cc(C5=CCCCO5)cnc4n3)c2)o1] | ¹H NMR (400 MHz, CD₃OD) δ 9.04 (d, J = 2.4 Hz, 1H), 8.82 (d, J = 2.4 Hz, 1H), 8.35 (m, 2H), 7.76 (ddd, J = 8.8, 4.4, 2.7 Hz, 1H), 7.35 (dd, J = 10.8, 9.0 Hz, 1H), 3.95-3.81 (m, 2H), 2.55 (s, 3H), 2.42 (s, 3H), 2.15 (d, J = 13.2 Hz, 1H), 2.05-1.91 (m, 1H), 1.80-1.59 (m, 3H). LCMS m/z = 434.2 (M + 1); RT = 1.7, Method 3. |
| 374 | 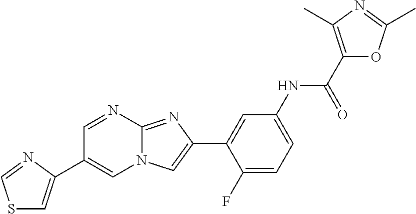 [SMILES: Cc1nc(C)c(C(=O)Nc2ccc(F)c(-c3cn4cc(-c5cscn5)cnc4n3)c2)o1] | ¹H NMR (400 MHz, CD₃OD) δ 9.63 (d, J = 2.3 Hz, 1H), 9.43 (d, J = 2.2 Hz, 1H), 9.20 (d, J = 1.8 Hz, 1H), 8.47 (d, J = 3.0 Hz, 1H), 8.39 (dd, J = 6.5, 2.7 Hz, 1H), 8.28 (d, J = 1.7 Hz, 1H), 7.80 (ddd, J = 9.0, 4.3, 2.6 Hz, 1H), 7.36 (dd, J = 10.7, 9.0 Hz, 1H), 2.57 (s, 3H), 2.48 (s, 3H). LCMS m/z = 435.2 (M + 1); RT = 1.54, Method 3. |
| 375 | 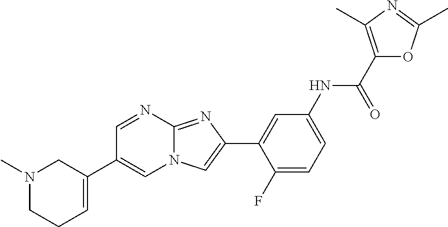 | LCMS m/z = 447.1 (M + 1); RT = 0.57, Method 2. |
| 376 | 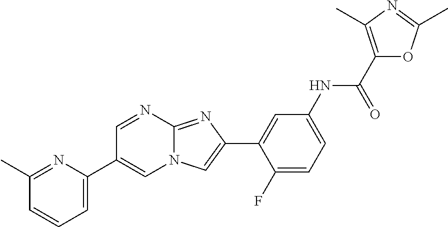 | ¹H NMR (400 MHz, CD₃OD) δ 9.61 (d, J = 2.4 Hz, 1H), 9.39 (d, J = 2.3 Hz, 1H), 8.43 (d, J = 3.3 Hz, 1H), 8.37 (dd, J = 6.6, 2.7 Hz, 1H), 7.93-7.79 (m, 3H), 7.38 (d, J = 7.4 Hz, 1H), 7.33 (dd, J = 10.8, 9.0 Hz, 1H), 2.66 (s, 3H), 2.57 (s, 3H), 2.48 (s, 3H). LCMS m/z = 443.3 (M + 1); RT = 3.04, Method 1. |
| 377 | 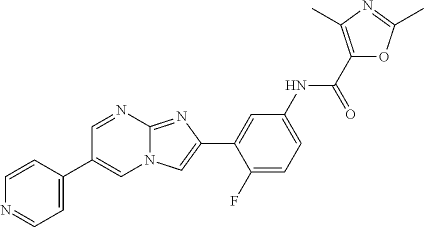 [SMILES: Cc1nc(C)c(C(=O)Nc2ccc(F)c(-c3cn4cc(-c5ccncc5)cnc4n3)c2)o1] | ¹H NMR (400 MHz, CD₃OD) δ 9.58 (d, J = 2.5 Hz, 1H), 9.13 (d, J = 2.6 Hz, 1H), 8.92-8.81 (m, 2H), 8.43-8.32 (m, 2H), 8.31-8.22 (m, 2H), 7.82 (ddd, J = 8.8, 4.4, 2.7 Hz, 1H), 7.28 (dd, J = 10.9, 8.9 Hz, 1H), 2.56 (s, 3H), 2.48 (s, 3H). LCMS m/z = 429.3 (M + 1); RT = 1.33, Method 3. |
| 378 | 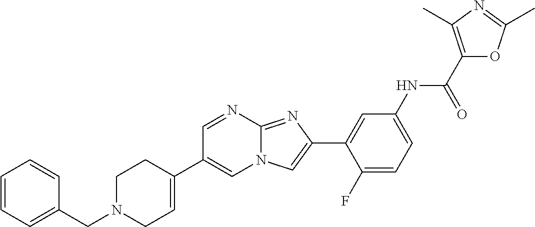 [SMILES: Cc1nc(C)c(C(=O)Nc2ccc(F)c(-c3cn4cc(C5=CCN(Cc6ccccc6)CC5)cnc4n3)c2)o1] | LCMS m/z = 523.2 (M + 1); RT = 0.7, Method 2. |
| 379 | 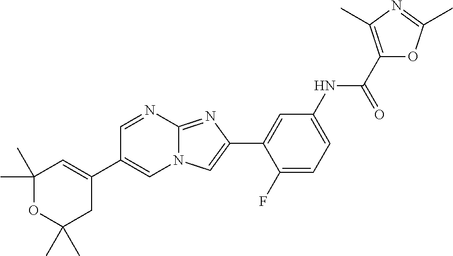 | LCMS m/z = 490.1 (M + 1); RT = 1.08, Method 2. |
| 380 | 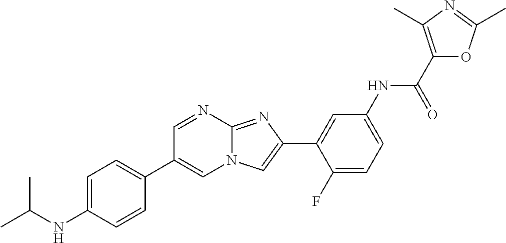 [SMILES: Cc1nc(C)c(C(=O)Nc2ccc(F)c(-c3cn4cc(-c5ccc(NC(C)C)cc5)cnc4n3)c2)o1] | LCMS m/z = 485.1 (M + 1); RT = 0.89, Method 2. |
| 381 | 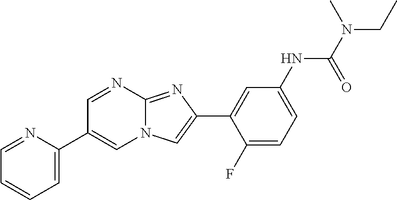 [SMILES: CCN(C)C(=O)Nc1ccc(F)c(-c2cn3cc(-c4ccccn4)cnc3n2)c1] | LCMS m/z = 391.1 (M + 1); RT = 0.83, Method 2. |
| 382 | 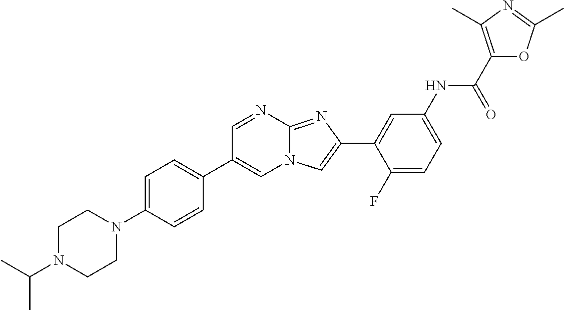 [SMILES: Cc1nc(C)c(C(=O)Nc2ccc(F)c(-c3cn4cc(-c5ccc(N6CCN(C(C)C)CC6)cc5)cnc4n3)c2)o1] | ¹H NMR (400 MHz, CD₃OD) δ 9.23 (d, J = 2.4 Hz, 1H), 9.09 (d, J = 2.4 Hz, 1H), 8.42-8.32 (m, 2H), 7.79 (ddd, J = 9.0, 4.4, 2.7 Hz, 1H), 7.76-7.69 (m, 2H), 7.34 (dd, J = 10.8, 8.9 Hz, 1H), 7.26-7.17 (m, 2H), 4.05 (d, J = 13.4 Hz, 2H), 3.70-3.56 (m, 3H), 3.33-3.32 (m, 2H), 3.20-3.06 (m, 2H), 2.56 (s, 3H), 2.48 (s, 3H), 1.44 (d, J = 6.7 Hz, 6H). LCMS m/z = 554.2 (M + 1); RT = 0.76, Method 2. |
| 383 | 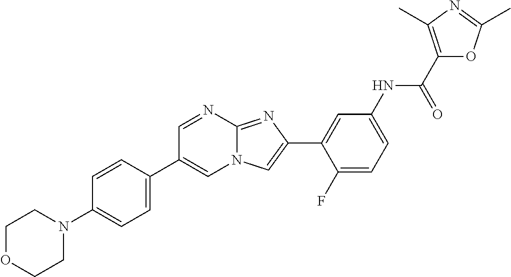 [SMILES: Cc1nc(C)c(C(=O)Nc2ccc(F)c(-c3cn4cc(-c5ccc(N6CCOCC6)cc5)cnc4n3)c2)o1] | ¹H NMR (400 MHz, CD₃OD) δ 9.29 (d, J = 2.4 Hz, 1H), 9.23 (d, J = 2.3 Hz, 1H), 8.43 (d, J = 2.6 Hz, 1H), 8.40 (dd, J = 6.6, 2.7 Hz, 1H), 7.75 (ddd, J = 9.0, 4.4, 2.7 Hz, 1H), 7.72-7.66 (m, 2H), 7.38 (dd, J = 10.6, 9.0 Hz, 1H), 7.19- 7.07 (m, 2H), 3.89-3.83 (m, 4H), 3.29- 3.22 (m, 4H), 2.56 (s, 3H), 2.48 (s, 3H). LCMS m/z = 513.1 (M + 1); RT = 0.99, Method 2. |
| 384 | 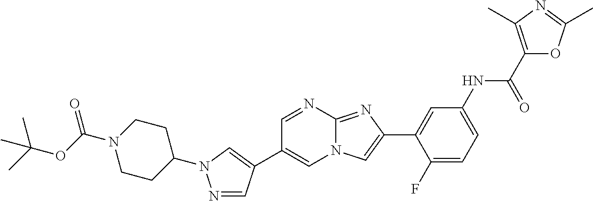 [SMILES: Cc1nc(C)c(C(=O)Nc2ccc(F)c(-c3cn4cc(-c5cnn(C6CCN(C(=O)OC(C)(C)C)CC6)c5)cnc4n3)c2)o1] | LCMS m/z = 601.3 (M + 1); RT = 1.07, Method 2. |
| 385 | 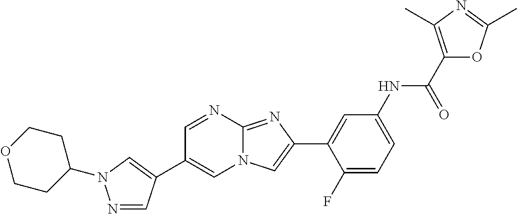 [SMILES: Cc1nc(C)c(C(=O)Nc2ccc(F)c(-c3cn4cc(-c5cnn(C6CCOCC6)c5)cnc4n3)c2)o1] | ¹H NMR (400 MHz. CD₃OD) δ 9.26 (d, J = 2.4 Hz, 1H), 9.16 (d, J = 2.4 Hz, 1H), 8.41-8.32 (m, 3H), 8.05 (s, 1H), 7.76 (ddd, J = 8.9, 4.5, 2.7 Hz, 1H), 7.35 (dd, J = 10.7, 9.0 Hz, 1H), 4.51 (tt, J = 8.8, 5.8 Hz, 1H), 4.10 (dt, J = 11.7, 3.3 Hz, 2H), 3.62 (ddd, J = 14.4, 10.9, 5.4 Hz, 2H), 2.56 (s, 3H), 2.48 (s, 3H), 2.13 (td, J = 9.4, 8.1, 3.8 Hz, 4H). LCMS m/z = 502.3 (M + 1); RT = 2.83, Method 1. |
| 386 | 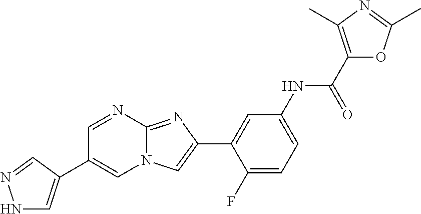 [SMILES: Cc1nc(C)c(C(=O)Nc2ccc(F)c(-c3cn4cc(-c5cn[nH]c5)cnc4n3)c2)o1] | LCMS m/z = 418 (M + 1); RT = 0.79, Method 2. |
| 387 | 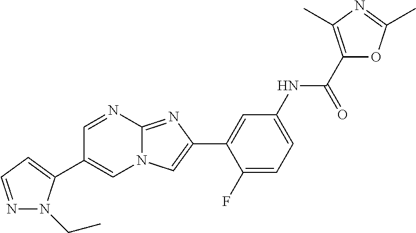 [SMILES: CCn1nccc1-c1cnc2nc(-c3cc(NC(=O)c4oc(C)nc4C)ccc3F)cn2c1] | LCMS m/z = 446 (M + 1); RT = 0.92, Method 2. |
| 388 | 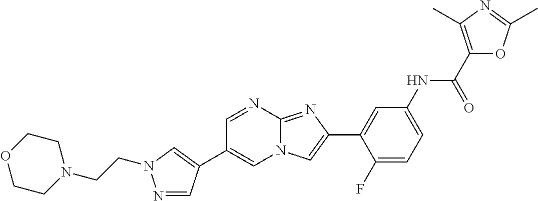 [SMILES: Cc1nc(C)c(C(=O)Nc2ccc(F)c(-c3cn4cc(-c5cnn(CCN6CCOCC6)c5)cnc4n3)c2)o1] | ¹H NMR (400 MHz, CD₃OD) δ 9.31 (d, J = 2.3 Hz, 1H), 9.16 (d, J = 2.3 Hz, 1H), 8.44-8.32 (m, 3H), 8.13 (s, 1H), 7.76 (ddd, J = 9.0, 4.5, 2.7 Hz, 1H), 7.37 (dd, J = 10.6, 9.0 Hz, 1H), 4.75 (t, J = 6.0 Hz, 2H), 3.95 (bs, 4H), 3.80 (t, J = 6.0 Hz, 2H), 3.46 (brs, 4H), 2.56 (s, 3H), 2.48 (s, 3H). LCMS m/z = 531.2 (M + 1); RT = 0.68, Method 2. |
| 389 | 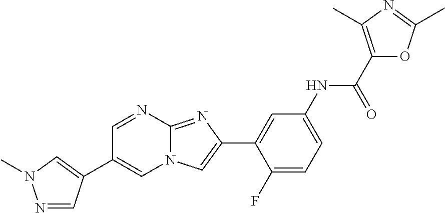 | ¹H NMR (400 MHz, CD₃OD) δ 9.29 (t, J = 2.6 Hz, 1H), 9.23-9.15 (m, 1H), 8.39 (q, J = 4.2, 3.6 Hz, 2H), 8.24 (d, J = 1.8 Hz, 1H), 8.03 (d, J = 1.7 Hz, 1H), 7.74 (ddd, J = 8.9, 4.6, 2.7 Hz, 1H), 7.44-7.30 (m, 1H), 4.00 (s, 3H), 2.56 (s, 3H), 2.48 (s, 3H). LCMS m/z = 432 (M + 1); RT = 0.84, Method 2. |
| 390 | 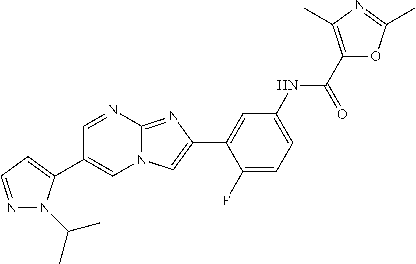 [SMILES: Cc1nc(C)c(C(=O)Nc2ccc(F)c(-c3cn4cc(-c5ccnn5C(C)C)cnc4n3)c2)o1] | LCMS m/z = 460.3 (M + 1); RT = 3.13, Method 1. |
| 391 | 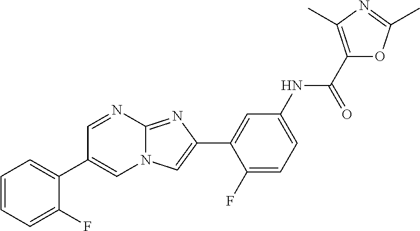 [SMILES: Cc1nc(C)c(C(=O)Nc2ccc(F)c(-c3cn4cc(-c5ccccc5F)cnc4n3)c2)o1] | ¹H NMR (400 MHz, CD₃OD) δ 9.25 (d, J = 2.3 Hz, 1H), 8.98 (t, J = 2.0 Hz, 1H), 8.41 (d, J = 3.2 Hz, 1H), 8.36 (dd, J = 6.7, 2.7 Hz, 1H), 7.81 (ddd, J = 9.0, 4.5, 2.7 Hz, 1H), 7.71 (td, J = 7.8, 1.7 Hz, 1H), 7.55 (tdd, J = 7.4, 5.1, 1.7 Hz, 1H), 7.44-7.27 (m, 3H), 2.56 (s, 3H), 2.48 (s, 3H). LCMS m/z = 446.4 (M + 1); RT = 1.79, Method 3. |
| 392 | 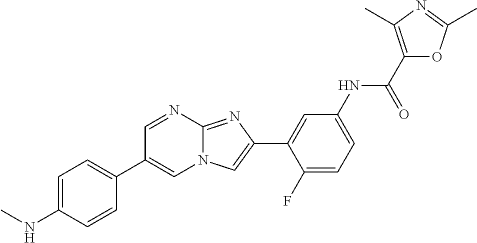 | LCMS m/z = 457.4 (M + 1); RT = 1.5, Method 3. |
| 393 | 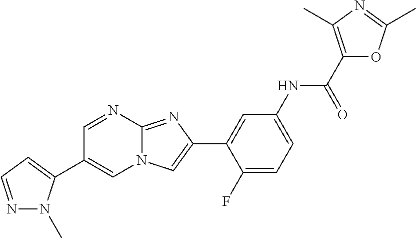 [SMILES: Cc1nc(C)c(C(=O)Nc2ccc(F)c(-c3cn4cc(-c5ccnn5C)cnc4n3)c2)o1] | ¹H NMR (400 MHz, CD₃OD) δ 9.28 (d, J = 2.4 Hz, 1H), 8.96 (d, J = 2.3 Hz, 1H), 8.45 (d, J = 3.1 Hz, 1H), 8.41 (dd, J = 6.5, 2.7 Hz, 1H), 7.81 (ddd, J = 9.1, 4.4, 2.8 Hz, 1H), 7.63 (d, J = 1.9 Hz, 1H), 7.36 (dd, J = 10.8, 8.9 Hz, 1H), 6.67 (d, J = 2.0 Hz, 1H), 4.00 (s, 3H), 2.57 (s, 3H), 2.48 (s, 3H). LCMS m/z = 432.2 (M + 1); RT = 1.5, Method 3. |
| 394 | 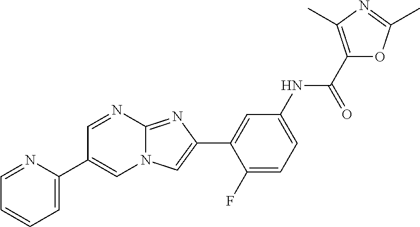 [SMILES: Cc1nc(C)c(C(=O)Nc2ccc(F)c(-c3cn4cc(-c5ccccn5)cnc4n3)c2)o1] | ¹H NMR (400 MHz, CD₃OD) δ 9.61 (d, J = 2.3 Hz, 1H), 9.40 (d, J = 2.3 Hz, 1H), 8.74 (dt, J = 4.7, 1.3 Hz, 1H), 8.42 (d, J = 3.4 Hz, 1H), 8.36 (dd, J = 6.6, 2.7 Hz, 1H), 8.06 (d, J = 8.0 Hz, 1H), 8.00 (td, J = 7.7, 1.7 Hz, 1H), 7.82 (ddd, J = 8.9, 4.3, 2.6 Hz, 1H), 7.48 (ddd, J = 7.4, 4.8, 1.2 Hz, 1H), 7.32 (dd, J = 10.8, 9.0 Hz, 1H), 2.57 (s, 3H), 2.48 (s, 3H). LCMS m/z = 429 (M + 1); RT = 0.88, Method 2. |
| 395 | 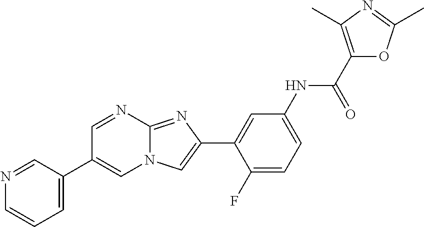 [SMILES: Cc1nc(C)c(C(=O)Nc2ccc(F)c(-c3cn4cc(-c5cccnc5)cnc4n3)c2)o1] | ¹H NMR (400 MHz, CD₃OD) δ 9.45 (d, J = 2.4 Hz, 1H), 9.20-9.08 (m, 2H), 8.81 (s, 1H), 8.58 (d, J = 8.0 Hz, 1H), 8.46-8.37 (m, 2H), 7.90 (dd, J = 8.0, 5.1 Hz, 1H), 7.81 (ddd, J = 8.8, 4.5, 2.8 Hz, 1H), 7.33 (dd, J = 10.9, 9.0 Hz, 1H), 2.57 (s, 3H), 2.48 (s, 3H). LCMS m/z = 429 (M + 1); RT = 0.71, Method 4. |
| 396 | 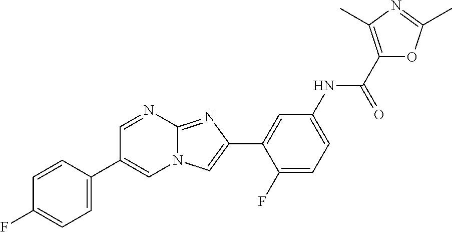 [SMILES: Cc1nc(C)c(C(=O)Nc2ccc(F)c(-c3cn4cc(-c5ccc(F)cc5)cnc4n3)c2)o1] | ¹H NMR (400 MHz, CD₃OD) δ 9.28 (d, J = 2.4 Hz, 1H), 9.09 (d, J = 2.4 Hz, 1H), 8.43-8.32 (m, 2H), 7.81 (dtd, J = 8.7, 4.5, 2.3 Hz, 3H), 7.40-7.27 (m, 3H), 2.56 (s, 3H), 2.48 (s, 3H). LCMS m/z = 446.4 (M + 1); RT = 1.79, Method 3. |
| 397 | 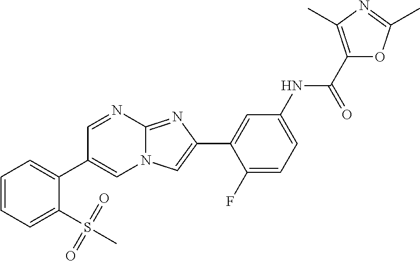 [SMILES: Cc1nc(C)c(C(=O)Nc2ccc(F)c(-c3cn4cc(-c5ccccc5S(C)(=O)=O)cnc4n3)c2)o1] | LCMS m/z = 506.2 (M + 1); RT = 1.68, Method 3. |
| 398 | 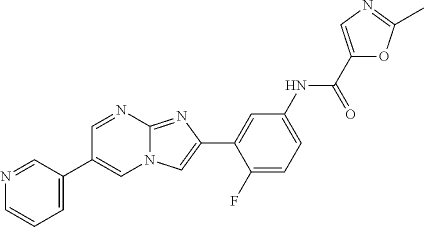 [SMILES: Cc1ncc(C(=O)Nc2ccc(F)c(-c3cn4cc(-c5cccnc5)cnc4n3)c2)o1] | LCMS m/z = 415.2 (M + 1); RT = 1.26, Method 3. |
| 399 | 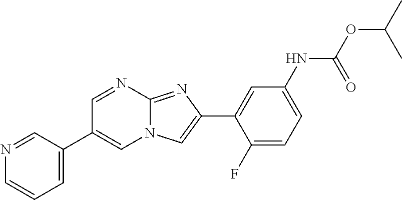 [SMILES: CC(C)OC(=O)Nc1ccc(F)c(-c2cn3cc(-c4cccnc4)cnc3n2)c1] | LCMS m/z = 392.2 (M + 1); RT = 1.42, Method 3. |
| 400 | 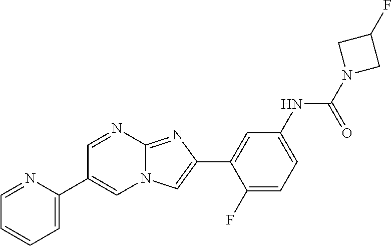 [SMILES: O=C(Nc1ccc(F)c(-c2cn3cc(-c4ccccn4)cnc3n2)c1)N1CC(F)C1] | LCMS m/z = 407.3 (M + 1); RT = 2.69, Method 1. |
| 401 | 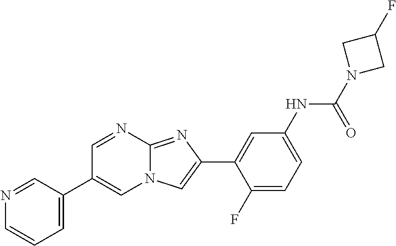 [SMILES: O=C(Nc1ccc(F)c(-c2cn3cc(-c4cccnc4)cnc3n2)c1)N1CC(F)C1] | LCMS m/z = 407.2 (M + 1); RT = 1.29, Method 3. |
| 402 | 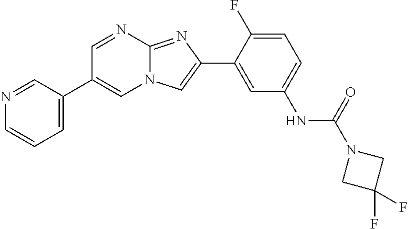 [SMILES: O=C(Nc1ccc(F)c(-c2cn3cc(-c4cccnc4)cnc3n2)c1)N1CC(F)(F)C1] | LCMS m/z = 425.2 (M + 1); RT = 1.29, Method 3. |
| 403 | 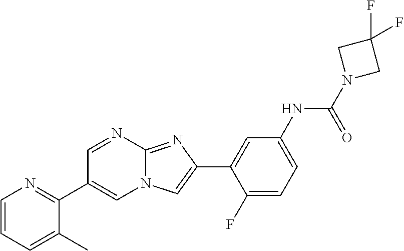 [SMILES: Cc1cccnc1-c1cnc2nc(-c3cc(NC(=O)N4CC(F)(F)C4)ccc3F)cn2c1] | LCMS m/z = 439 (M + 1); RT = 0.82, Method 4. |
| 404 | 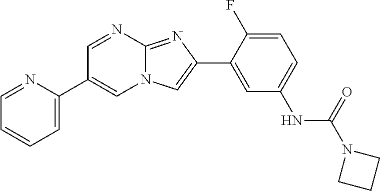 [SMILES: O=C(Nc1ccc(F)c(-c2cn3cc(-c4ccccn4)cnc3n2)c1)N1CCC1] | LCMS m/z = 389.3 (M + 1); RT = 2.68, Method 1. |
| 405 | 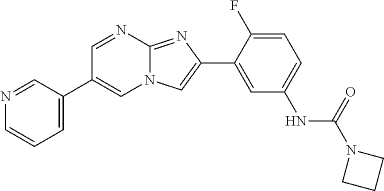 [SMILES: O=C(Nc1ccc(F)c(-c2cn3cc(-c4cccnc4)cnc3n2)c1)N1CCC1] | ¹H NMR (400 MHz, CD₃OD) δ 9.43 (d, J = 2.6 Hz, 1H), 9.12 (dd, J = 11.3, 2.4 Hz, 2H), 8.78 (dd, J = 5.1, 1.5 Hz, 1H), 8.52 (dt, J = 8.1, 1.9 Hz, 1H), 8.39 (d, J = 3.2 Hz, 1H), 8.11 (dd, J = 6.7, 2.8 Hz, 1H), 7.85 (dd, J = 8.1, 5.2 Hz, 1H), 7.56 (ddd, J = 8.9, 4.4, 2.7 Hz, 1H), 7.23 (dd, J = 10.8, 9.0 Hz, 1H), 4.18- 4.05 (m, 4H), 2.41-2.27 (m, 2H). LCMS m/z = 389.2 (M + 1); RT = 1.26, Method 3. |
| 406 | 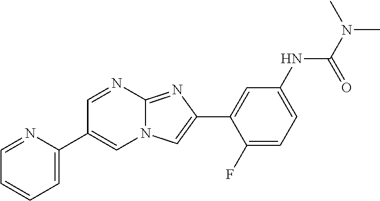 | LCMS m/z = 389.2 (M + 1); RT = 1.26, Method 3. |
| 407 | 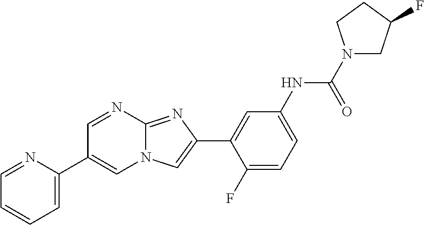 [SMILES: O=C(Nc1ccc(F)c(-c2cn3cc(-c4ccccn4)cnc3n2)c1)N1CC[C@@H](F)C1] | ¹H NMR (400 MHz, CD₃OD) δ 9.67 (d, J = 2.3 Hz, 1H), 9.47 (d, J = 2.3 Hz, 1H), 8.76 (dt, J = 4.8, 1.4 Hz, 1H), 8.44 (d, J = 3.0 Hz, 1H), 8.14-8.05 (m, 2H), 8.01 (td, J = 7.8, 1.7 Hz, 1H), 7.57 (ddd, J = 8.9, 4.5, 2.7 Hz, 1H), 7.50 (ddd. J = 7.4, 4.8, 1.2 Hz, 1H), 7.27 (dd, J = 10.8, 9.0 Hz, 1H), 5.35 (dt, J = 52.3, 3.6 Hz, 1H), 3.89-3.67 (m, 3H), 3.67-3.52 (m, 1H), 2.41-2.09 (m, 2H). LCMS m/z = 421 (M + 1); RT = 0.76, Method 2. |
| 408 | 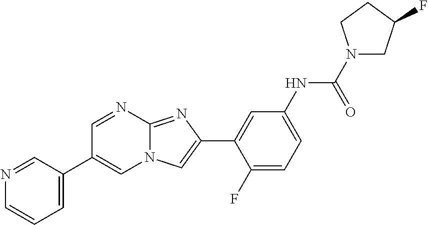 [SMILES: O=C(Nc1ccc(F)c(-c2cn3cc(-c4cccnc4)cnc3n2)c1)N1CC[C@@H](F)C1] | LCMS m/z = 421.2 (M + 1); RT = 1.22, Method 3. |
| 409 | 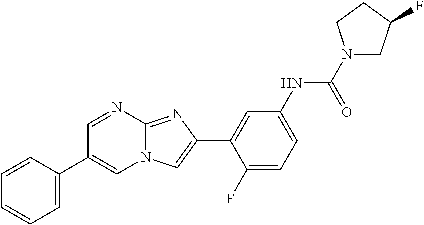 [SMILES: O=C(Nc1ccc(F)c(-c2cn3cc(-c4ccccc4)cnc3n2)c1)N1CC[C@@H](F)C1] | ¹H NMR (400 MHz, CD₃OD) δ 9.15 (d, J = 2.4 Hz, 1H), 8.92 (d, J = 2.4 Hz, 1H), 8.28 (d, J = 3.8 Hz, 1H), 8.05 (dd, J = 6.6, 2.7 Hz, 1H), 7.75 (d, J = 7.9 Hz, 2H), 7.66-7.40 (m, 4H), 7.18 (dd, J = 10.9, 9.0 Hz, 1H), 5.35 (d, J = 52.8 Hz, 1H), 3.88-3.67 (m, 2H), 3.61 (ddt, J = 17.2, 10.3, 5.2 Hz, 2H), 2.41-2.02 (m, 2H). LCMS m/z = 420.3 (M + 1); RT = 3.04, Method 1. |
| 410 | 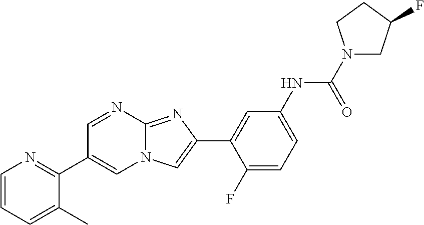 [SMILES: Cc1cccnc1-c1cnc2nc(-c3cc(NC(=O)N4CC[C@@H](F)C4)ccc3F)cn2c1] | ¹H NMR (400 MHz, CD₃OD) δ 9.13 (d, J = 2.4 Hz, 1H), 8.81 (d, J = 2.4 Hz, 1H), 8.55 (d, J = 4.0 Hz, 1H), 8.32 (d, J = 3.8 Hz, 1H), 8.08 (dd, J = 6.6, 2.7 Hz, 1H), 7.87 (d, J = 7.6 Hz, 1H), 7.62 (ddd, J = 8.8, 4.4, 2.9 Hz, 1H), 7.44 (dd, J = 7.8, 4.8 Hz, 1H), 7.19 (dd, J = 11.0, 9.0 Hz, 1H), 5.35 (d, J = 52.8 Hz, 1H), 3.75 (d, J = 9.6 Hz, 2H), 3.66-3.55 (m, 2H), 2.51 (s, 3H), 2.37-2.08 (m, 2H). LCMS m/z = 435.1 (M + 1); RT = 0.76, Method 4. |
| 411 | 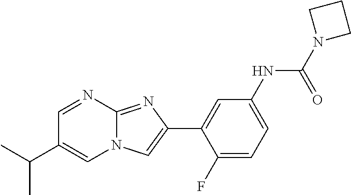 [SMILES: CC(C)c1cnc2nc(-c3cc(NC(=O)N4CCC4)ccc3F)cn2c1] | ¹H NMR (400 MHz, CD₃OD) δ 8.72 (d, J = 2.4 Hz, 1H), 8.58 (d, J = 2.4 Hz, 1H), 8.15 (d, J = 3.9 Hz, 1H), 7.97 (dd, J = 6.5, 2.8 Hz, 1H), 7.62 (ddd, J = 8.8, 4.5, 2.9 Hz, 1H), 7.14 (dd, J = 11.0, 9.0 Hz, 1H), 4.11 (t, J = 7.6 Hz, 4H), 3.08 (p, J = 7.0 Hz, 1H), 2.40-2.24 (m, 2H), 1.38 (d, J = 7.1 Hz, 6H). LCMS m/z = 354.2 (M + 1); RT = 1.33, Method 3. |
| 412 | 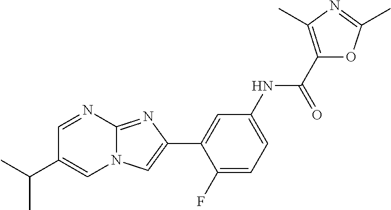 [SMILES: Cc1nc(C)c(C(=O)Nc2ccc(F)c(-c3cn4cc(C(C)C)cnc4n3)c2)o1] | ¹H NMR (400 MHz, CD₃OD) δ 8.73 (d, J = 2.3 Hz, 1H), 8.59 (d, J = 2.4 Hz, 1H), 8.24 (dd, J = 6.7, 2.7 Hz, 1H), 8.18 (d, J = 3.9 Hz, 1H), 7.83 (ddd, J = 8.9, 4.4, 2.8 Hz, 1H), 7.23 (dd, J = 10.9, 8.9 Hz, 1H), 3.09 (p, J = 6.9 Hz, 1H), 2.56 (s, 3H), 2.46 (s, 3H), 1.38 (d, J = 6.9 Hz, 6H). LCMS m/z = 394.1 (M + 1); RT = 0.9, Method 2. |
| 413 | 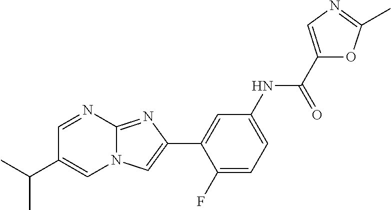 [SMILES: Cc1ncc(C(=O)Nc2ccc(F)c(-c3cn4cc(C(C)C)cnc4n3)c2)o1] | ¹H NMR (600 MHz, CD₃OD) δ 8.73 (dd, J = 2.4, 0.9 Hz, 1H), 8.59 (d, J = 2.5 Hz, 1H), 8.25 (dd, J = 6.6, 2.8 Hz, 1H), 8.19 (d, J = 3.8 Hz, 1H), 7.89 (ddd, J = 8.9, 4.4, 2.7 Hz, 1H), 7.77 (s, 1H), 7.25 (dd, J = 10.9, 8.9 Hz, 1H), 3.15-3.03 (m, 1H), 2.60 (s, 3H), 1.39 (d, J = 7.0 Hz, 6H). LCMS m/z = 380 (M + 1); RT = 0.86, Method 2. |
| 414 | 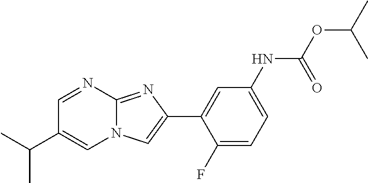 [SMILES: CC(C)OC(=O)Nc1ccc(F)c(-c2cn3cc(C(C)C)cnc3n2)c1] | ¹H NMR (400 MHz, CD₃OD) δ 8.72 (d, J = 2.4 Hz, 1H), 8.58 (d, J = 2.4 Hz, 1H), 8.15 (d, J = 3.9 Hz, 1H), 7.97 (dd, J = 6.6, 2.8 Hz, 1H), 7.74-7.62 (m, 1H), 7.16 (dd, J = 11.0, 8.9 Hz, 1H), 4.98 (dq, J = 12.3, 6.2 Hz, 1H), 3.09 (dq, J = 13.9, 6.7 Hz, 1H), 1.38 (d, J = 7.0 Hz, 6H), 1.32 (d, J = 6.2 Hz, 6H). LCMS m/z = 357.1 (M + 1); RT = 1, Method 2. |
| 415 | 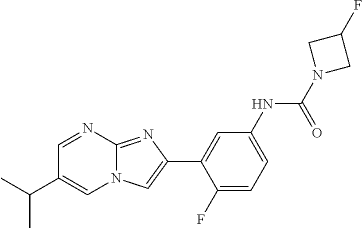 [SMILES: CC(C)c1cnc2nc(-c3cc(NC(=O)N4CC(F)C4)ccc3F)cn2c1] | ¹H NMR (400 MHz, CD₃OD) δ 8.76- 8.67 (m, 1H), 8.57 (d, J = 2.4 Hz, 1H), 8.15 (d, J = 3.9 Hz, 1H), 7.99 (dd, J = 6.5, 2.8 Hz, 1H), 7.62 (ddd, J = 9.0, 4.4, 2.8 Hz, 1H), 7.15 (dd, J = 11.0, 9.0 Hz, 1H), 5.37 (dtt, J = 57.1, 6.1, 3.2 Hz, 1H), 4.39 (dddd, J = 20.8, 10.2, 6.1, 1.4 Hz, 2H), 4.14 (dddd, J = 24.6, 10.3, 3.2, 1.4 Hz, 2H), 3.08 (p, J = 6.9 Hz, 1H), 1.38 (d, J = 7.0 Hz, 6H). LCMS m/z = 372.1 (M + 1); RT = 0.84, Method 4. |
| 416 | 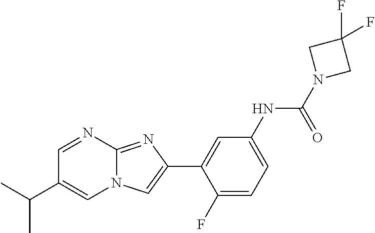 [SMILES: CC(C)c1cnc2nc(-c3cc(NC(=O)N4CC(F)(F)C4)ccc3F)cn2c1] | ¹H NMR (600 MHz, CD₃OD) δ 8.77- 8.69 (m, 1H), 8.58 (d, J = 2.4 Hz, 1H), 8.16 (d, J = 3.8 Hz, 1H), 8.03 (dd, J = 6.6, 2.8 Hz, 1H), 7.63 (ddd, J = 8.9, 4.4, 2.8 Hz, 1H), 7.17 (dd, J = 10.9, 8.9 Hz, 1H), 4.43 (t, J = 12.3 Hz, 4H), 3.08 (p, J = 6.9 Hz, 1H), 1.38 (d, J = 7.0 Hz, 6H). LCMS m/z = 390 (M + 1); RT = 0.89, Method 2. |
| 417 | 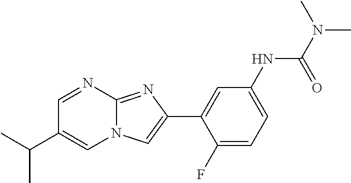 [SMILES: CC(C)c1cnc2nc(-c3cc(NC(=O)N(C)C)ccc3F)cn2c1] | ¹H NMR (400 MHz, CD₃OD) δ 8.73 (d, J = 2.2 Hz, 1H), 8.58 (d, J = 2.4 Hz, 1H), 8.16 (d, J = 3.9 Hz, 1H), 7.96 (dd, J = 6.6, 2.8 Hz, 1H), 7.51 (ddd, J = 8.9, 4.4, 2.8 Hz, 1H), 7.15 (dd, J = 11.0, 8.9 Hz, 1H), 3.13-3.06 (m, 1H), 3.05 (s, 6H), 1.38 (d, J = 6.9 Hz, 6H). LCMS m/z = 342.1 (M + 1); RT = 0.78, Method 2. |
| 418 | 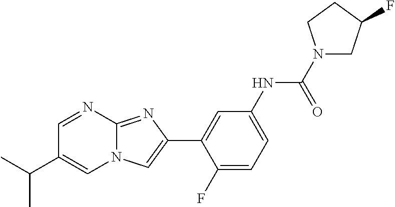 [SMILES: CC(C)c1cnc2nc(-c3cc(NC(=O)N4CC[C@@H](F)C4)ccc3F)cn2c1] | ¹H NMR (600 MHz, CD₃OD) δ 8.72 (d, J = 2.4 Hz, 1H), 8.58 (d, J = 2.6 Hz, 1H), 8.16 (d, J = 3.8 Hz, 1H), 8.00 (dd, J = 6.6, 2.8 Hz, 1H), 7.58 (ddd, J = 8.9, 4.4, 2.8 Hz, 1H), 7.16 (dd, J = 11.0, 8.9 Hz, 1H), 5.33 (dt, J = 52.9, 3.6 Hz, 1H), 3.85-3.66 (m, 3H), 3.65-3.53 (m, 1H), 3.09 (hept, J = 7.0 Hz, 1H), 2.36-2.26 (m, 1H), 2.19 (dddd, J = 41.8, 13.8, 9.1, 3.7 Hz, 1H), 1.38 (d, J = 7.0 Hz, 6H). LCMS m/z = 386.1 (M + 1); RT = 0.83, Method 2. |
| 419 | 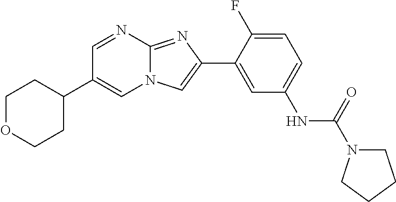 [SMILES: O=C(Nc1ccc(F)c(-c2cn3cc(C4CCOCC4)cnc3n2)c1)N1CCCC1] | ¹H NMR (400 MHz, CD₃OD) δ 8.75 (d, J = 2.4 Hz, 1H), 8.58 (d, J = 2.4 Hz, 1H), 8.16 (d, J = 3.8 Hz, 1H), 7.99 (dd, J = 6.7, 2.8 Hz, 1H), 7.57 (ddd, J = 8.8, 4.5, 2.8 Hz, 1H), 7.15 (dd, J = 11.0, 8.9 Hz, 1H), 4.13-4.04 (m, 2H), 3.61 (td, J = 11.5, 2.4 Hz, 2H), 3.54-3.44 (m, 4H), 2.99 (tt, J = 11.6, 4.1 Hz, 1H), 1.99 (p, J = 3.7 Hz, 4H), 1.95-1.78 (m, 4H). LCMS m/z = 410.1 (M + 1); RT = 0.76, Method 2. |
| 420 | 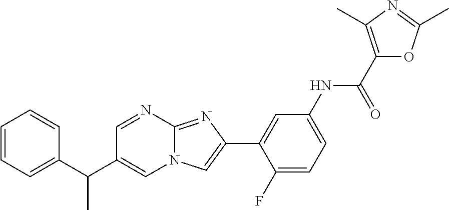 [SMILES: Cc1nc(C)c(C(=O)Nc2ccc(F)c(-c3cn4cc(C(C)c5ccccc5)cnc4n3)c2)o1] | ¹H NMR (400 MHz, CD₃OD) δ 8.99 (s, 1H), 8.73 (d, J = 2.1 Hz, 1H), 8.39-8.31 (m, 2H), 7.76 (dt, J = 7.5, 3.5 Hz, 1H), 7.40-7.31 (m, 5H), 7.27 (dq, J = 5.7, 2.9 Hz, 1H), 4.43 (q, J = 7.2 Hz, 1H), 2.55 (s, 3H), 2.47 (s, 3H), 1.77 (d, J = 7.2 Hz, 3H). LCMS m/z = 456.2 (M + 1); RT = 1.72, Method 3. |
| 421 | 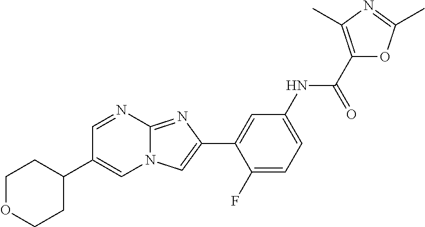 [SMILES: Cc1nc(C)c(C(=O)Nc2ccc(F)c(-c3cn4cc(C5CCOCC5)cnc4n3)c2)o1] | ¹H NMR (400 MHz, CD₃OD) δ 8.77 (d, J = 2.3 Hz, 1H), 8.60 (d, J = 2.4 Hz, 1H), 8.26 (dd, J = 6.5, 2.8 Hz, 1H), 8.21 (d, J = 3.9 Hz, 1H), 7.84 (ddd, J = 8.8, 4.4, 2.7 Hz, 1H), 7.25 (dd, J = 10.9, 9.0 Hz, 1H), 4.15-4.04 (m, 2H), 3.61 (td, J = 11.6, 2.5 Hz, 2H), 3.00 (tt, J = 11.3, 4.1 Hz, 1H), 2.56 (s, 3H), 2.47 (s, 3H), 1.97-1.79 (m, 4H). LCMS m/z = 436 (M + 1); RT = 0.83, Method 2. |
| 422 | 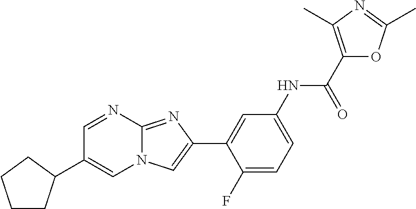 [SMILES: Cc1nc(C)c(C(=O)Nc2ccc(F)c(-c3cn4cc(C5CCCC5)cnc4n3)c2)o1] | ¹H NMR (400 MHz, CD₃OD) δ 8.74 (d, J = 2.3 Hz, 1H), 8.58 (d, J = 2.4 Hz, 1H), 8.25 (dd, J = 6.6, 2.7 Hz, 1H), 8.18 (d, J = 3.8 Hz, 1H), 7.83 (ddd, J = 8.8, 4.5, 2.9 Hz, 1H), 7.24 (dd, J = 10.9, 9.0 Hz, 1H), 3.15 (tt, J = 9.4, 6.8 Hz, 1H), 2.56 (s, 3H), 2.47 (s, 3H), 2.27-2.14 (m, 2H), 1.96-1.85 (m, 2H), 1.85-1.76 (m, 2H), 1.69 (tdd, J = 11.7, 9.3, 4.8 Hz, 2H). LCMS m/z = 420.4 (M + 1); RT = 1.68, Method 3. |
| 423 | 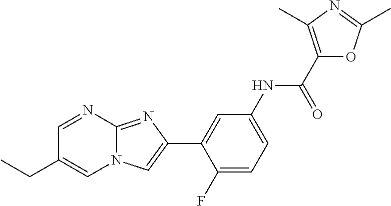 [SMILES: CCc1cnc2nc(-c3cc(NC(=O)c4oc(C)nc4C)ccc3F)cn2c1] | ¹H NMR (400 MHz, CD₃OD) δ 8.72 (d, J = 2.4 Hz, 1H), 8.54 (d, J = 2.4 Hz, 1H), 8.25 (dd, J = 6.7, 2.7 Hz, 1H), 8.19 (d, J = 3.8 Hz, 1H), 7.84 (ddd, J = 9.1, 4.6, 2.8 Hz, 1H), 7.25 (dd, J = 11.0, 8.9 Hz, 1H), 2.77 (q, J = 7.6 Hz, 2H), 2.56 (s, 3H), 2.47 (s, 3H), 1.35 (t, J = 7.7 Hz, 3H). LCMS m/z = 380.3 (M + 1); RT = 1.44, Method 3. |
| 424 | 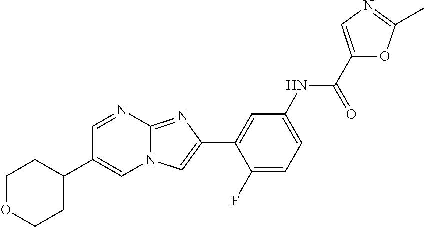 [SMILES: Cc1ncc(C(=O)Nc2ccc(F)c(-c3cn4cc(C5CCOCC5)cnc4n3)c2)o1] | LCMS m/z = 422.1 (M + 1); RT = 0.82, Method 2. |
| 425 | 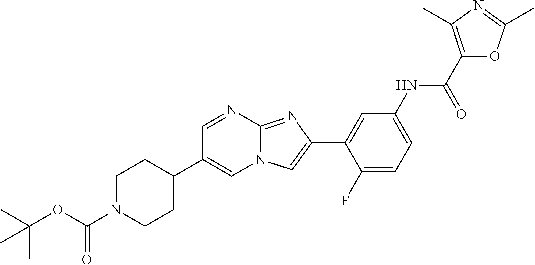 [SMILES: Cc1nc(C)c(C(=O)Nc2ccc(F)c(-c3cn4cc(C5CCN(C(=O)OC(C)(C)C)CC5)cnc4n3)c2)o1] | LCMS m/z = 535.3 (M + 1); RT = 1.66, Method 3. |
| 426 | 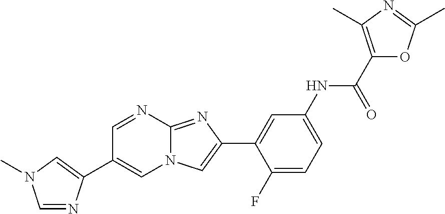 | ¹H NMR (400 MHz, DMSO-d₆) δ 10.36 (brs, 1H), 9.52 (brs, 1H), 9.14 (brs, 1H), 9.02 (brs, 1H), 8.74 (brs, 1H), 8.41 (brs, 1H), 8.19 (brs, 1H), 7.80 (brs, 1H), 7.37 (t, J = 9.54 Hz, 1H), 3.93 (br. s., 3H), 2.40 (m, 3H), 2.32 (s, 3H). LCMS m/z = 432 (M + 1); RT = 0.71, Method 2. |
| 427 | 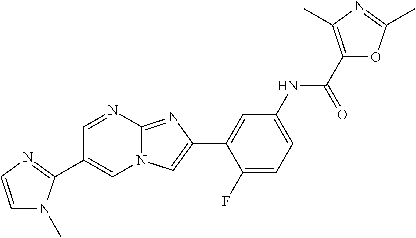 [SMILES: Cc1nc(C)c(C(=O)Nc2ccc(F)c(-c3cn4cc(-c5nccn5C)cnc4n3)c2)o1] | LCMS m/z = 432.1 (M + 1); RT = 0.63, Method 2. |
| 428 | 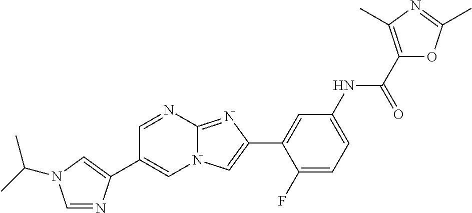 [SMILES: Cc1nc(C)c(C(=O)Nc2ccc(F)c(-c3cn4cc(-c5cn(C(C)C)cn5)cnc4n3)c2)o1] | LCMS m/z = 460.4 (M + 1); RT = 2.62, Method 1. |
| 429 | 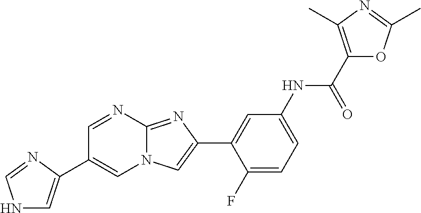 [SMILES: Cc1nc(C)c(C(=O)Nc2ccc(F)c(-c3cn4cc(-c5c[nH]cn5)cnc4n3)c2)o1] | ¹H NMR (400 MHz, DMSO-d₆) δ 12.43 (brs, 1H), 10.33 (s, 1H), 9.30 (d, J = 1.76 Hz, 1H), 9.04 (d, J = 1.76 Hz, 1H), 8.73 (d, J = 4.52 Hz, 1H), 8.35 (d, J = 4.02 Hz, 1H), 7.84-7.77 (m, 2H), 7.35- 7.30 (m, 1H), 2.49 (s, 3H), 2.41 (s, 3H). |
| 430 | 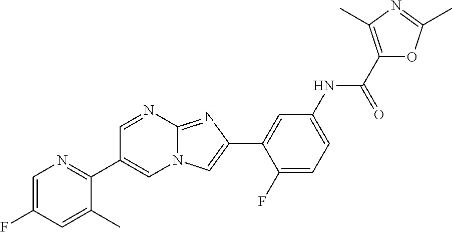 [SMILES: Cc1nc(C)c(C(=O)Nc2ccc(F)c(-c3cn4cc(-c5ncc(F)cc5C)cnc4n3)c2)o1] | ¹H NMR (400 MHz, CD₃OD) δ 9.31 (d, J = 2.3 Hz, 1H), 9.07 (d, J = 2.3 Hz, 1H), 8.52 (d, J = 2.7 Hz, 1H), 8.47 (d, J = 2.9 Hz, 1H), 8.41 (dd, J = 6.6, 2.8 Hz, 1H), 7.81 (ddd, J = 9.0, 4.4, 2.7 Hz, 1H), 7.73 (dd, J = 9.2, 2.7 Hz, 1H), 7.37 (dd, J = 10.7, 9.0 Hz, 1H), 2.57 (s, 3H), 2.56 (s, 3H), 2.49 (s, 3H). LCMS m/z = 461.4 (M + 1); RT = 1.63, Method 3. |
| 431 | 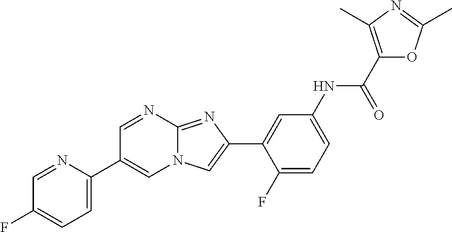 [SMILES: Cc1nc(C)c(C(=O)Nc2ccc(F)c(-c3cn4cc(-c5ccc(F)cn5)cnc4n3)c2)o1] | LCMS m/z = 447.2 (M + 1); RT = 1.68, Method 3. |
| 432 | 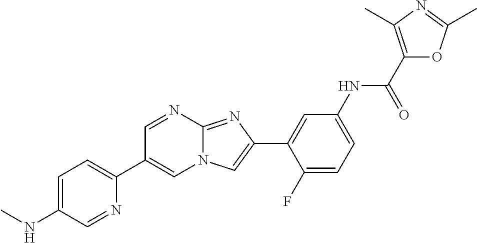 | ¹H NMR (400 MHz, CD₃OD) δ 9.44 (d, J = 2.4 Hz, 1H), 9.30 (d, J = 2.4 Hz, 1H), 8.43 (d, J = 3.0 Hz, 1H), 8.39 (dd, J = 6.6, 2.7 Hz, 1H), 8.10 (d, J = 2.9 Hz, 1H), 7.90 (d, J = 8.8 Hz, 1H), 7.78 (ddd, J = 9.0, 4.5, 2.7 Hz, 1H), 7.39- 7.27 (m, 2H), 2.91 (s, 3H), 2.56 (s, 3H), 2.48 (s, 3H). LCMS m/z = 458.4 (M + 1); RT = 1.46, Method 3. |
| 433 | 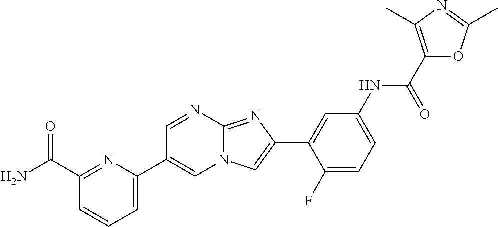 [SMILES: Cc1nc(C)c(C(=O)Nc2ccc(F)c(-c3cn4cc(-c5cccc(C(N)=O)n5)cnc4n3)c2)o1] | ¹H NMR (400 MHz, CD₃OD) δ 9.31 (d, J = 2.3 Hz, 1H), 9.07 (d, J = 2.3 Hz, 1H), 8.52 (d, J = 2.7 Hz, 1H), 8.47 (d, J = 2.9 Hz, 1H), 8.41 (dd, J = 6.6, 2.8 Hz, 1H), 7.81 (ddd, J = 9.0, 4.4, 2.7 Hz, 1H), 7.73 (dd, J = 9.2, 2.7 Hz, 1H), 7.37 (dd, J = 10.7, 9.0 Hz, 1H), 2.57 (s, 3H), 2.56 (s, 3H), 2.49 (s, 3H). LCMS m/z = 472.2 (M + 1); RT = 1.52, Method 3. |
| 434 | 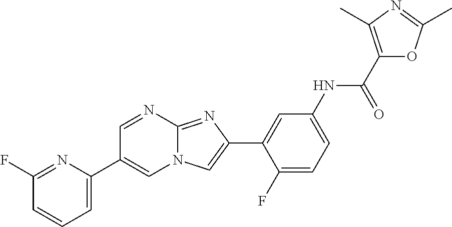 [SMILES: Cc1nc(C)c(C(=O)Nc2ccc(F)c(-c3cn4cc(-c5cccc(F)n5)cnc4n3)c2)o1] | ¹H NMR (400 MHz, CD₃OD) δ 9.63 (d, J = 2.3 Hz, 1H), 9.35 (d, J = 2.3 Hz, 1H), 8.37 (dt, J = 19.3, 3.0 Hz, 2H), 8.11 (q, J = 8.0 Hz, 1H), 7.88-7.74 (m, 2H), 7.58 (t, J = 7.4 Hz, 1H), 7.37-7.26 (m, 1H), 2.57 (s, 3H), 2.48 (s, 3H). LCMS m/z = 447.2 (M + 1); RT = 1.76, Method 3. |
| 435 | 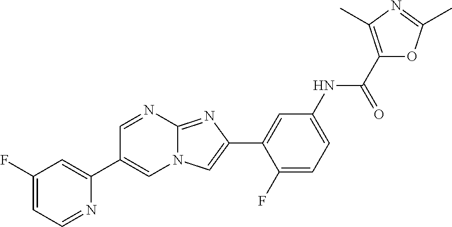 [SMILES: Cc1nc(C)c(C(=O)Nc2ccc(F)c(-c3cn4cc(-c5cc(F)ccn5)cnc4n3)c2)o1] | ¹H NMR (400 MHz, CD₃OD) δ 9.73 (d, J = 2.3 Hz, 1H), 9.50 (d, J = 2.3 Hz, 1H), 8.76 (dd, J = 8.5, 5.7 Hz, 1H), 8.48 (d, J = 3.1 Hz, 1H), 8.40 (dd, J = 6.5, 2.7 Hz, 1H), 7.96 (dd, J = 10.2, 2.3 Hz, 1H), 7.81 (ddd, J = 8.9, 4.4, 2.7 Hz, 1H), 7.41-7.24 (m, 2H), 2.56 (s, 3H), 2.48 (s, 3H). LCMS m/z = 447.3 (M + 1); RT = 1.7, Method 3. |
| 436 | 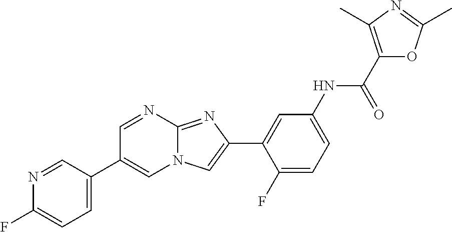 [SMILES: Cc1nc(C)c(C(=O)Nc2ccc(F)c(-c3cn4cc(-c5ccc(F)nc5)cnc4n3)c2)o1] | ¹H NMR (400 MHz, CD₃OD) δ 9.40 (d, J = 2.4 Hz, 1H), 9.15 (d, J = 2.4 Hz, 1H), 8.65 (d, J = 2.6 Hz, 1H), 8.45 (d, J = 3.0 Hz, 1H), 8.43-8.32 (m, 2H), 7.81 (ddd, J = 8.9, 4.5, 2.7 Hz, 1H), 7.41-7.27 (m, 2H), 2.57 (s, 3H), 2.48 (s, 3H). LCMS m/z = 447.4 (M + 1); RT = 1.61, Method 3. |
| 437 | 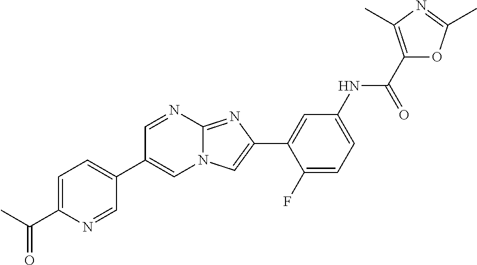 [SMILES: CC(=O)c1ccc(-c2cnc3nc(-c4cc(NC(=O)c5oc(C)nc5C)ccc4F)cn3c2)cn1] | ¹H NMR (400 MHz, CD₃OD) δ 9.48 (d, J = 2.4 Hz, 1H), 9.18 (d, J = 2.3 Hz, 1H), 9.12 (d, J = 2.3 Hz, 1H), 8.44 (d, J = 3.3 Hz, 1H), 8.39 (ddd, J = 8.3, 5.2, 2.4 Hz, 2H), 8.20 (d, J = 8.1 Hz, 1H), 7.82 (ddd, J = 8.9, 4.5, 2.7 Hz, 1H), 7.34 (dd, J = 10.8, 9.0 Hz, 1H), 2.75 (s, 3H), 2.57 (s, 3H), 2.48 (s, 3H). LCMS m/z = 471.4 (M + 1); RT = 1.63, Method 3. |
| 438 | 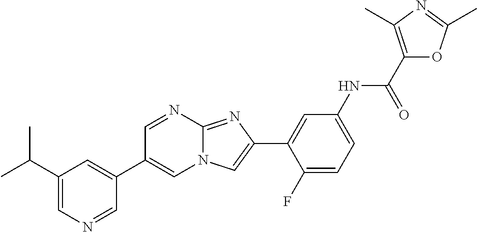 [SMILES: Cc1nc(C)c(C(=O)Nc2ccc(F)c(-c3cn4cc(-c5cncc(C(C)C)c5)cnc4n3)c2)o1] | ¹H NMR (400 MHz, CD₃OD) δ 9.16 (d, J = 2.4 Hz, 1H), 8.84 (d, J = 8.4 Hz, 2H), 8.76 (d, J = 2.4 Hz, 1H), 8.43 (q, J = 3.0 Hz, 2H), 8.12 (d, J = 6.1 Hz, 1H), 7.82 (ddd, J = 8.9, 4.5, 2.8 Hz, 1H), 7.33 (dd, J = 10.8, 8.9 Hz, 1H), 3.31- 3.28 (m, 1H), 2.57 (s, 3H), 2.48 (s, 3H), 1.35 (d, J = 6.8 Hz, 6H). LCMS m/z = 471.4 (M + 1); RT = 1.44, Method 3. |
| 439 | 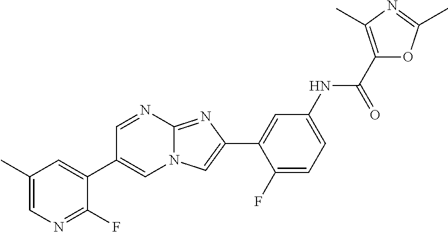 | ¹H NMR (400 MHz, CD₃OD) δ 9.29 (d, J = 2.4 Hz, 1H), 8.96 (t, J = 2.0 Hz, 1H), 8.45-8.30 (m, 2H), 8.18-8.06 (m, 2H), 7.82 (ddd, J = 9.0, 4.4, 2.7 Hz, 1H), 7.31 (dd, J = 10.8, 9.0 Hz, 1H), 2.56 (s, 3H), 2.48 (s, 3H), 2.46 (s, 3H). LCMS m/z = 461.4 (M + 1); RT = 1.7, Method 3. |
| 440 | 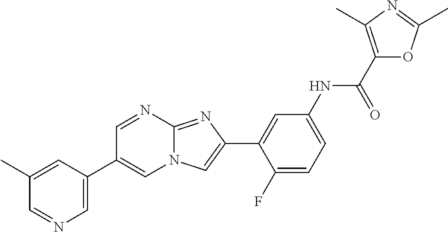 | LCMS m/z = 443.4 (M + 1); RT = 1.37, Method 3. |
| 441 | 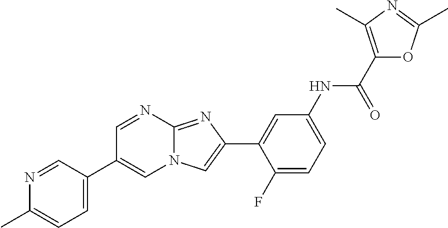 | LCMS m/z = 443.4 (M + 1); RT = 1.35, Method 3. |
| 442 | 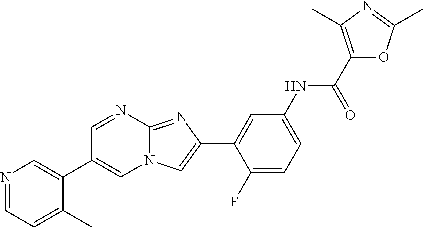 [SMILES: Cc1nc(C)c(C(=O)Nc2ccc(F)c(-c3cn4cc(-c5cnccc5C)cnc4n3)c2)o1] | ¹H NMR (400 MHz, CD₃OD) δ 9.23 (d, J = 2.4 Hz, 1H), 8.94-8.74 (m, 3H), 8.43 (dd, J = 5.1, 2.9 Hz, 2H), 8.06 (d, J = 5.8 Hz, 1H), 7.80 (ddd, J = 8.9, 4.5, 2.7 Hz, 1H), 7.33 (dd, J = 10.7, 9.0 Hz, 1H), 2.69 (s, 3H), 2.56 (s, 3H), 2.47 (s, 3H). LCMS m/z = 443.4 (M + 1); RT = 1.31, Method 3. |
| 443 | 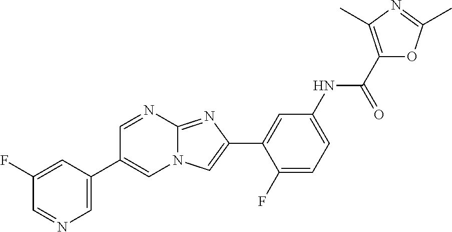 [SMILES: Cc1nc(C)c(C(=O)Nc2ccc(F)c(-c3cn4cc(-c5cncc(F)c5)cnc4n3)c2)o1] | LCMS m/z = 447.3 (M + 1); RT = 1.61, Method 3. |
| 444 | 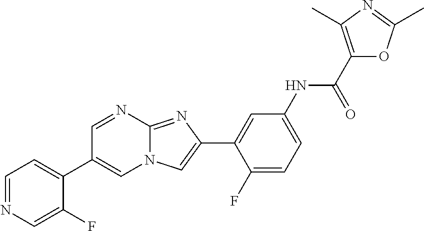 [SMILES: Cc1nc(C)c(C(=O)Nc2ccc(F)c(-c3cn4cc(-c5ccncc5F)cnc4n3)c2)o1] | ¹H NMR (400 MHz, CD₃OD) δ 9.43 (d, J = 2.4 Hz, 1H), 9.04 (t, J = 1.8 Hz, 1H), 8.69 (d, J = 2.7 Hz, 1H), 8.58 (d, J = 5.1 Hz, 1H), 8.42 (d, J = 3.4 Hz, 1H), 8.37 (dd, J = 6.7, 2.8 Hz, 1H), 7.90- 7.77 (m, 2H), 7.31 (dd, J = 10.9, 8.9 Hz, 1H), 2.56 (s, 3H), 2.48 (s, 3H). LCMS m/z = 447.4 (M + 1); RT = 1.59, Method 3. |
| 445 | 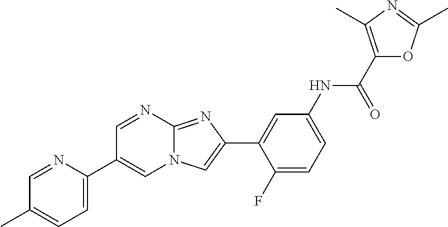 | ¹H NMR (400 MHz, CD₃OD) δ 9.74 (d, J = 2.2 Hz, 1H), 9.59 (d, J = 2.3 Hz, 1H), 8.65 (d, J = 2.1 Hz, 1H), 8.57 (d, J = 2.4 Hz, 1H), 8.42 (dd, J = 6.5, 2.8 Hz, 1H), 8.05 (d, J = 8.1 Hz, 1H). 7.94 (dd, J = 8.0, 2.1 Hz, 1H), 7.77 (ddd, J = 9.1, 4.5, 2.6 Hz, 1H), 7.42 (dd, J = 10.6, 9.0 Hz, 1H), 2.57 (s, 3H), 2.48 (s, 3H), 2.48 (s, 3H). LCMS m/z = 443.4 (M + 1); RT = 1.65, Method 3. |
| 446 | 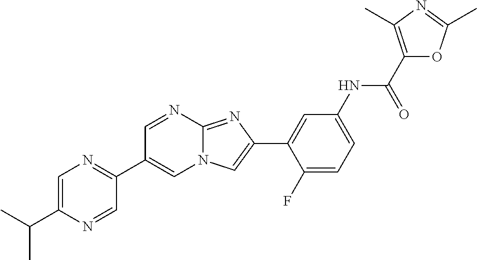 [SMILES: Cc1nc(C)c(C(=O)Nc2ccc(F)c(-c3cn4cc(-c5cnc(C(C)C)cn5)cnc4n3)c2)o1] | LCMS m/z = 472.3 (M + 1); RT = 1.78, Method 3. |
| 447 | 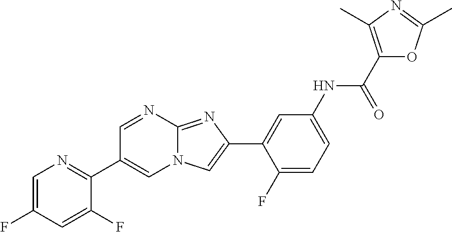 [SMILES: Cc1nc(C)c(C(=O)Nc2ccc(F)c(-c3cn4cc(-c5ncc(F)cc5F)cnc4n3)c2)o1] | ¹H NMR (600 MHz, CD₃OD) δ 9.39 (d, J = 2.4 Hz, 1H), 9.18-9.09 (m, 1H), 8.49 (d, J = 2.4 Hz, 1H), 8.28 (d, J = 3.8 Hz, 1H), 8.26-8.15 (m, 1H), 7.77- 7.68 (m, 2H), 7.18 (dd, J = 10.8, 8.9 Hz, 1H), 2.47 (s, 3H), 2.38 (s, 3H). LCMS m/z = 465.4 (M + 1); RT = 1.78, Method 3. |
| 448 | 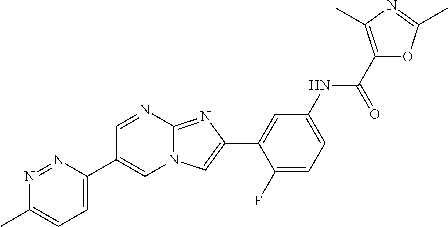 | ¹H NMR (400 MHz, CD₃OD) δ 9.64 (d, J = 2.5 Hz, 1H), 9.35 (d, J = 2.4 Hz, 1H), 8.39 (d, J = 3.7 Hz, 1H), 8.34 (dd, J = 6.7, 2.8 Hz, 1H), 8.23 (d, J = 8.8 Hz, 1H), 7.85 (ddd, J = 9.0, 4.5, 2.8 Hz, 1H), 7.79 (d, J = 8.7 Hz, 1H), 7.28 (dd, J = 10.9, 8.9 Hz, 1H), 2.77 (s, 3H), 2.57 (s, 3H), 2.48 (s, 3H). LCMS m/z = 444 (M + 1); RT = 0.77, Method 2. |
| 449 | 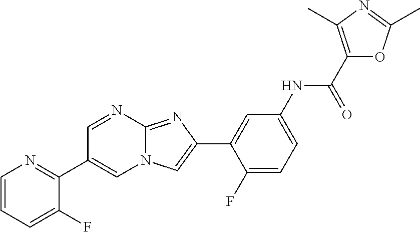 [SMILES: Cc1nc(C)c(C(=O)Nc2ccc(F)c(-c3cn4cc(-c5ncccc5F)cnc4n3)c2)o1] | ¹H NMR (600 MHz, CD₃OD) δ 9.56 (d, J = 2.4 Hz, 1H), 9.37-9.30 (m, 1H), 8.61 (dt, J = 4.6, 1.5 Hz, 1H), 8.41 (d, J = 3.5 Hz, 1H), 8.35 (dd, J = 6.6, 2.8 Hz, 1H), 7.87-7.77 (m, 2H), 7.54 (dt, J = 8.5, 4.3 Hz, 1H), 7.30 (dd, J = 10.8, 8.9 Hz, 1H), 2.57 (s, 3H), 2.48 (s, 3H). LCMS m/z = 447.4 (M + 1); RT = 1.72, Method 3. |
| 450 | 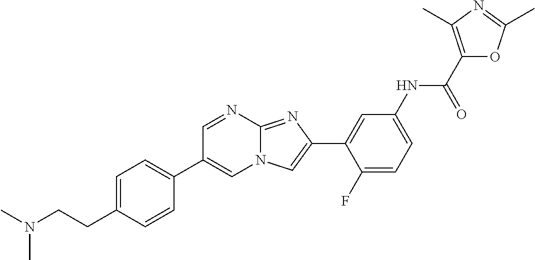 | LCMS m/z = 499.4 (M + 1); RT = 1.42, Method 3. |
| 451 | 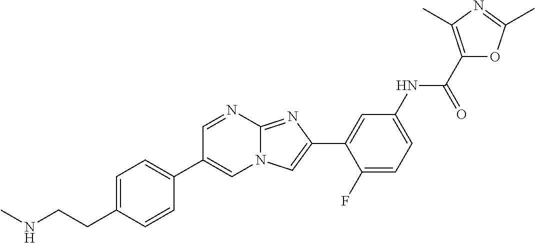 | ¹H NMR (600 MHz, CD₃OD) δ 9.23 (d, J = 2.5 Hz, 1H), 9.00 (d, J = 2.4 Hz, 1H), 8.39-8.34 (m, 2H), 7.81 (ddd, J = 8.9, 4.4, 2.7 Hz, 1H), 7.79-7.76 (m, 2H), 7.54-7.48 (m, 2H), 7.30 (dd, J = 10.8, 8.9 Hz, 1H), 3.34-3.32 (m, 2H), 3.09 (dd, J = 8.9, 6.9 Hz, 2H), 2.77 (s, 3H), 2.56 (s, 3H), 2.48 (s, 3H). LCMS m/z = 485.4 (M + 1); RT = 1.41, Method 3. |
| 452 | 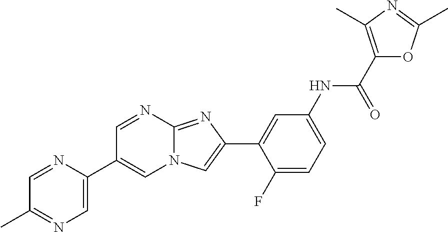 | ¹H NMR (400 MHz, CD₃OD) δ 9.78 (d, J = 2.0 Hz, 1H), 9.55 (s, 1H), 9.43-9.39 (m, 1H), 9.15 (d, J = 1.3 Hz, 1H), 8.69 (d, J = 2.3 Hz, 1H), 8.50 (d, J = 2.7 Hz, 1H), 8.37 (dd, J = 6.5, 2.6 Hz, 1H), 7.37-7.24 (m, 1H), 2.67 (s, 3H), 2.57 (s, 3H), 2.49 (s, 3H). LCMS m/z = 444.4 (M + 1); RT = 1.66, Method 3. |
| 453 | 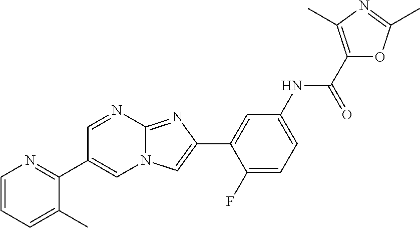 [SMILES: Cc1nc(C)c(C(=O)Nc2ccc(F)c(-c3cn4cc(-c5ncccc5C)cnc4n3)c2)o1] | ¹H NMR (400 MHz, CD₃OD) δ 9.14 (d, J = 2.4 Hz, 1H), 8.82 (d, J = 2.6 Hz, 1H), 8.55 (dd, J = 4.8, 1.7 Hz, 1H), 8.37-8.29 (m, 2H), 7.91-7.82 (m, 2H), 7.43 (dd, J = 7.8, 4.8 Hz, 1H), 7.27 (dd, J = 10.9, 8.9 Hz, 1H), 2.57 (s, 3H), 2.51 (s, 3H), 2.48 (s, 3H). LCMS m/z = 443 (M + 1); RT = 0.83, Method 2. |
| 454 | 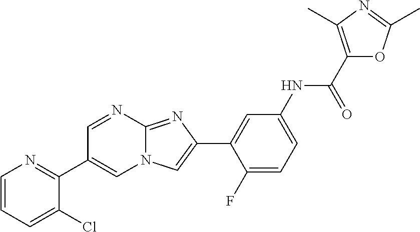 [SMILES: Cc1nc(C)c(C(=O)Nc2ccc(F)c(-c3cn4cc(-c5ncccc5Cl)cnc4n3)c2)o1] | ¹H NMR (400 MHz, CD₃OD) δ 9.63- 9.53 (m, 1H), 9.32-9.21 (m, 1H), 8.72 (dd, J = 4.4, 1.3 Hz, 1H), 8.52 (t, J = 3.0 Hz, 1H), 8.41 (dt, J = 6.6, 1.9 Hz, 1H), 8.12 (dd, J = 8.2, 1.3 Hz, 1H), 7.80 (ddt, J = 9.9, 4.1, 1.7 Hz, 1H), 7.55 (dd, J = 8.2, 4.7 Hz, 1H), 7.37 (ddd, J = 10.7, 8.9, 1.7 Hz, 1H), 2.57 (s, 3H), 2.48 (s, 3H). LCMS m/z = 463 (M + 1); RT = 0.92, Method 2. |
| 455 | 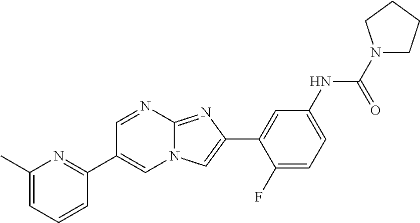 [SMILES: Cc1cccc(-c2cnc3nc(-c4cc(NC(=O)N5CCCC5)ccc4F)cn3c2)n1] | LCMS m/z = 417.1 (M + 1); RT = 0.89, Method 2. |
| 456 | 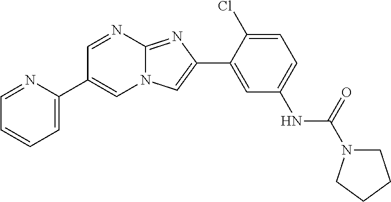 [SMILES: O=C(Nc1ccc(Cl)c(-c2cn3cc(-c4ccccn4)cnc3n2)c1)N1CCCC1] | LCMS m/z = 419.2 (M + 1); RT = 1.44, Method 3. |
| 457 | 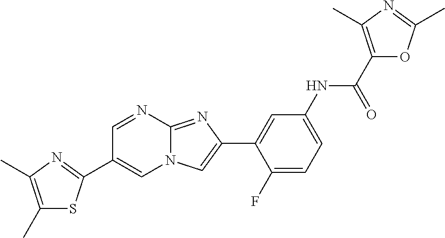 | LCMS m/z = 463.3 (M + 1); RT = 3.18, Method 1. |
| 458 | 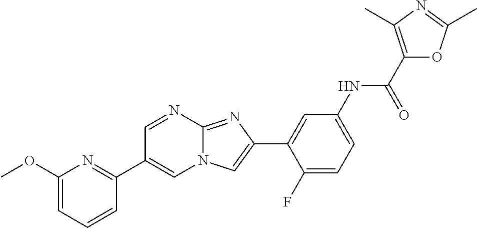 | LCMS m/z = 459.4 (M + 1); RT = 1.89, Method 3. |
| 459 | 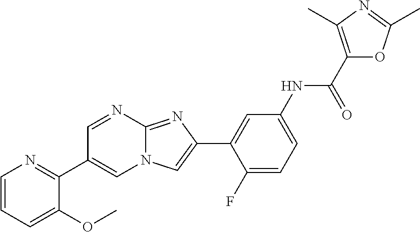 [SMILES: COc1cccnc1-c1cnc2nc(-c3cc(NC(=O)c4oc(C)nc4C)ccc3F)cn2c1] | LCMS m/z = 459.3 (M + 1); RT = 2.98, Method 1. |
| 460 | 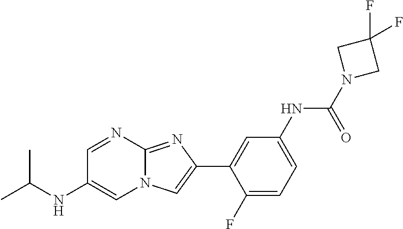 [SMILES: CC(C)Nc1cnc2nc(-c3cc(NC(=O)N4CC(F)(F)C4)ccc3F)cn2c1] | ¹H NMR (400 MHz, DMSO-d₆) δ 9.04 (s, 1H), 8.27-8.24 (m, 2H), 8.06-8.03 (m, 2H), 7.65-7.58 (m, 1H), 7.19 (dd, J = 11.04, 9.03 Hz, 1H), 5.68 (d, J = 7.53 Hz, 1H), 4.39 (t, J = 12.80 Hz, 4H), 3.41-3.38 (m., 1H), 1.19-1.11 (m, 6H). LCMS m/z = 405.1 (M + 1); RT = 0.78, Method 2. |
| 461 | 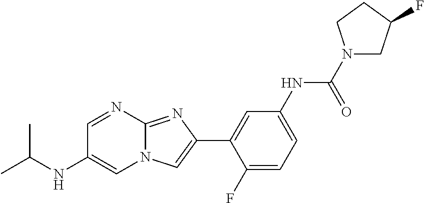 [SMILES: CC(C)Nc1cnc2nc(-c3cc(NC(=O)N4CC[C@@H](F)C4)ccc3F)cn2c1] | LCMS m/z = 401.3 (M + 1); RT = 2.64, Method 1. |
| 462 | 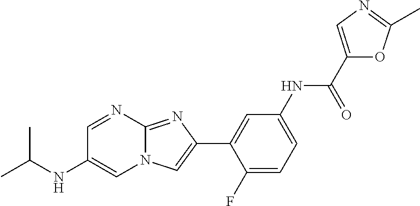 [SMILES: Cc1ncc(C(=O)Nc2ccc(F)c(-c3cn4cc(NC(C)C)cnc4n3)c2)o1] | LCMS m/z = 395.1 (M + 1); RT = 0.76, Method 2. |
| 463 | 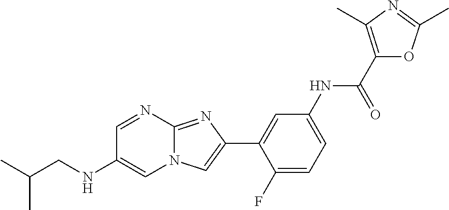 [SMILES: Cc1nc(C)c(C(=O)Nc2ccc(F)c(-c3cn4cc(NCC(C)C)cnc4n3)c2)o1] | LCMS m/z = 423.4 (M + 1); RT = 1.59, Method 3. |
| 464 | 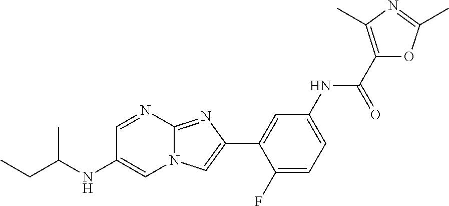 [SMILES: CCC(C)Nc1cnc2nc(-c3cc(NC(=O)c4oc(C)nc4C)ccc3F)cn2c1] | LCMS m/z = 423.4 (M + 1); RT = 1.55, Method 3. |
| 465 | 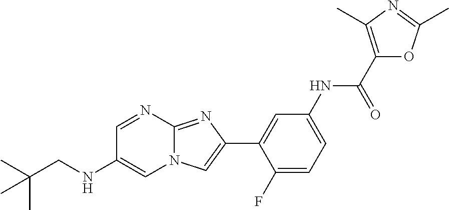 [SMILES: Cc1nc(C)c(C(=O)Nc2ccc(F)c(-c3cn4cc(NCC(C)(C)C)cnc4n3)c2)o1] | LCMS m/z = 437.4 (M + 1); RT = 1.63, Method 3. |
| 466 | 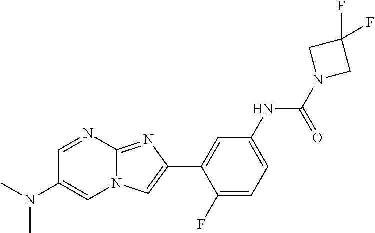 [SMILES: CN(C)c1cnc2nc(-c3cc(NC(=O)N4CC(F)(F)C4)ccc3F)cn2c1] | ¹H NMR (400 MHz, DMSO-d₆) δ 9.06 (brs., 1H), 8.62 (d, J = 3.01 Hz, 1H), 8.31-8.33 (m, 2H), 8.11 (d, J = 4.52 Hz, 1H), 7.64-7.60 (m, 1H), 7.24-7.19 (m, 1H), 4.40 (t, J = 12.80 Hz, 4H), 2.89 (s, 6H). LCMS m/z = 391 (M + 1); RT = 0.72, Method 2. |
| 467 | 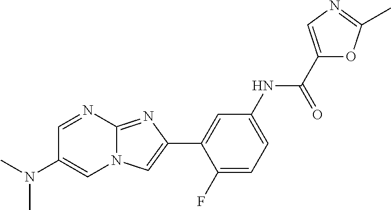 | LCMS m/z = 381 (M + 1); RT = 0.68, Method 2. |
| 468 | 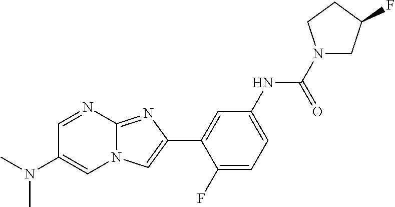 [SMILES: CN(C)c1cnc2nc(-c3cc(NC(=O)N4CC[C@@H](F)C4)ccc3F)cn2c1] | LCMS m/z = 387 (M + 1); RT = 0.66, Method 2. |
| 469 | 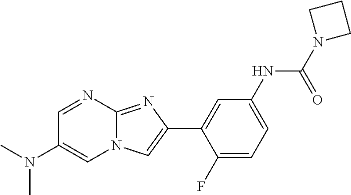 [SMILES: CN(C)c1cnc2nc(-c3cc(NC(=O)N4CCC4)ccc3F)cn2c1] | LCMS m/z = 355.1 (M + 1); RT = 0.64, Method 4. |
| 470 | 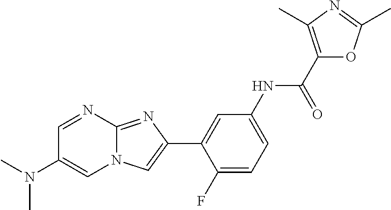 | ¹H NMR (400 MHz, DMSO-d₆) δ 10.30 (s, 1H), 8.68 (dd, J = 7.1, 2.8 Hz, 1H), 8.64 (d, J = 3.0 Hz, 1H), 8.32 (d, J = 3.0 Hz, 1H), 8.13 (d, J = 4.3 Hz, 1H), 7.74 (ddd, J = 8.8, 4.5, 2.8 Hz, 1H), 7.29 (dd, J = 11.0, 8.9 Hz, 1H), 2.89 (s, 6H), 2.50 (s, 3H), 2.40 (s, 3H). LCMS m/z = 395.2 (M + 1); RT = 1.35, Method 3. |
| 471 | 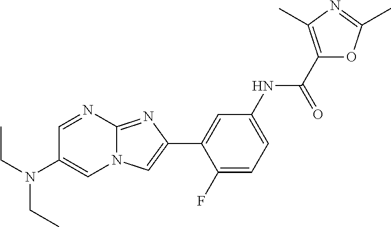 [SMILES: CCN(CC)c1cnc2nc(-c3cc(NC(=O)c4oc(C)nc4C)ccc3F)cn2c1] | LCMS m/z = 423.4 (M + 1); RT = 1.54, Method 3. |
| 472 | 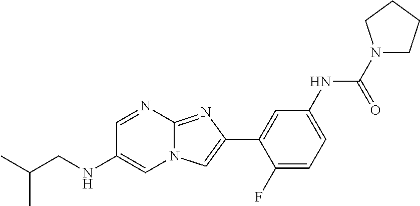 [SMILES: CC(C)CNc1cnc2nc(-c3cc(NC(=O)N4CCCC4)ccc3F)cn2c1] | ¹H NMR (400 MHz, CD₃OD) δ 8.61 (d, J = 2.8 Hz, 1H), 8.19 (d, J = 2.5 Hz, 1H), 8.14 (d, J = 2.8 Hz, 1H), 8.02 (dd, J = 6.6, 2.7 Hz, 1H), 7.47 (ddd, J = 8.9, 4.4, 2.7 Hz, 1H), 7.27 (dd, J = 10.6, 9.0 Hz, 1H), 3.59-3.45 (m, 4H), 2.94 (d, J = 6.9 Hz, 2H), 2.05-1.95 (m, 5H), 1.07 (d, J = 6.7 Hz, 6H). LCMS m/z = 397.3 (M + 1); RT = 1.54, Method 3. |
| 473 | 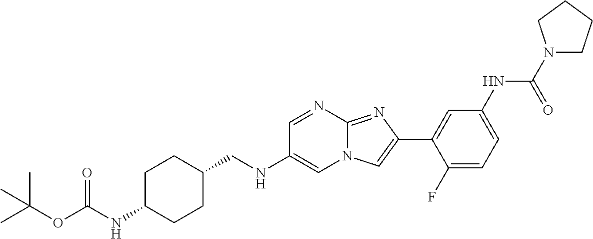 | ¹H NMR (400 MHz, CD₃OD) δ 8.64 (d, J = 2.8 Hz, 1H), 8.22 (d, J = 2.3 Hz, 1H), 8.17 (d, J = 2.8 Hz, 1H), 8.04 (dd, J = 6.6, 2.7 Hz, 1H), 7.46 (ddd, J = 8.9, 4.5, 2.7 Hz, 1H), 7.30 (dd, J = 10.5, 9.0 Hz, 1H), 3.65 (s, 1H), 3.56-3.41 (m, 4H), 3.05 (d, J = 6.9 Hz, 2H), 2.01 (t, J = 6.4 Hz, 4H), 1.85 (s, 1H), 1.76-1.56 (m, 6H), 1.46 (s, 11H). LCMS m/z = 397.3 (M + 1); RT = 1.54, Method 3. |
| 474 | 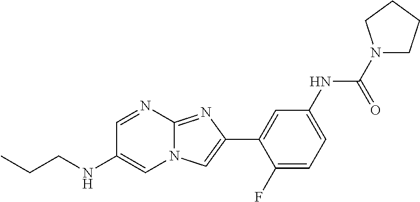 [SMILES: CCCNc1cnc2nc(-c3cc(NC(=O)N4CCCC4)ccc3F)cn2c1] | ¹H NMR (400 MHz, CD₃OD) δ 8.62 (d, J = 2.8 Hz, 1H), 8.21 (d, J = 2.4 Hz, 1H), 8.16 (d, J = 2.8 Hz, 1H), 8.03 (dd, J = 6.6, 2.7 Hz, 1H), 7.46 (ddd, J = 8.9, 4.5, 2.7 Hz, 1H), 7.29 (dd, J = 10.6, 9.0 Hz, 1H), 3.59-3.41 (m, 4H), 3.15-2.97 (m, 2H), 2.01 (t, J = 6.5 Hz, 4H), 1.76 (q, J = 7.2 Hz, 2H), 1.08 (t, J = 7.4 Hz, 3H). LCMS m/z = 383.1 (M + 1); RT = 0.77, Method 2. |
| 475 | 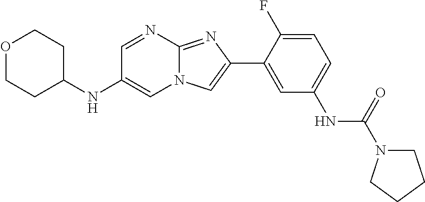 [SMILES: O=C(Nc1ccc(F)c(-c2cn3cc(NC4CCOCC4)cnc3n2)c1)N1CCCC1] | LCMS m/z = 425.1 (M + 1); RT = 0.69, Method 2. |
| 476 | 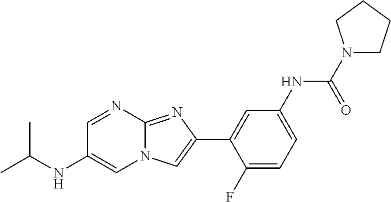 [SMILES: CC(C)Nc1cnc2nc(-c3cc(NC(=O)N4CCCC4)ccc3F)cn2c1] | ¹H NMR (400 MHz, CD₃OD) δ 8.56 (d, J = 2.8 Hz, 1H), 8.18 (dd, J = 9.5, 2.6 Hz, 2H), 8.02 (dd, J = 6.6, 2.7 Hz, 1H), 7.46 (ddd, J = 8.9, 4.4, 2.7 Hz, 1H), 7.28 (dd, J = 10.6, 9.0 Hz, 1H), 3.62- 3.45 (m, 5H), 2.00 (t, J = 6.4 Hz, 4H), 1.30 (d, J = 6.3 Hz, 6H). LCMS m/z = 383.2 (M + 1); RT = 1.39, Method 3. |
| 477 | 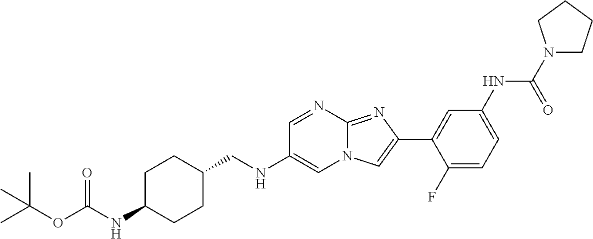 [SMILES: CC(C)(C)OC(=O)N[C@H]1CC[C@H](CNc2cnc3nc(-c4cc(NC(=O)N5CCCC5)ccc4F)cn3c2)CC1] | ¹H NMR (400 MHz, CD₃OD) δ 8.62 (d, J = 2.8 Hz, 1H), 8.21 (d, J = 2.3 Hz, 1H), 8.16 (d, J = 2.8 Hz, 1H), 8.03 (dd, J = 6.6, 2.6 Hz, 1H), 7.46 (ddd, J = 8.9, 4.4, 2.7 Hz, 1H), 7.29 (dd, J = 10.5, 9.1 Hz, 1H), 3.60-3.43 (m, 4H), 3.35-3.31 (m, 1H), 2.98 (d, J = 6.7 Hz, 2H), 1.99 (q, J = 10.8, 8.6 Hz, 8H), 1.68 (s, 1H), 1.44 (s, 9H), 1.32-1.10 (m, 4H). LCMS m/z = 552.4 (M + 1); RT = 1.7, Method 3. |
| 478 | 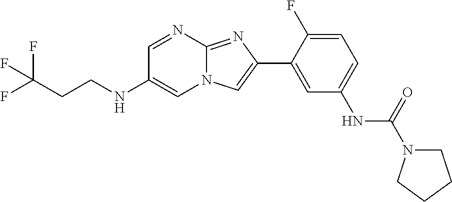 [SMILES: O=C(Nc1ccc(F)c(-c2cn3cc(NCCC(F)(F)F)cnc3n2)c1)N1CCCC1] | ¹H NMR (400 MHz, CD₃OD) δ 8.59 (d, J = 2.8 Hz, 1H), 8.25 (d, J = 2.8 Hz, 1H), 8.21 (d, J = 2.5 Hz, 1H), 8.02 (dd, J = 6.5, 2.7 Hz, 1H), 7.47 (ddd, J = 9.1, 4.5, 2.7 Hz, 1H), 7.27 (dd, J = 10.6, 8.9 Hz, 1H), 3.54-3.47 (m, 4H), 3.43 (t, J = 7.0 Hz, 2H), 2.62 (qt, J = 10.9, 6.9 Hz, 2H), 2.01-1.98 (m, 4H). LCMS m/z = 437 (M + 1); RT = 0.81, Method 2. |
| 479 | 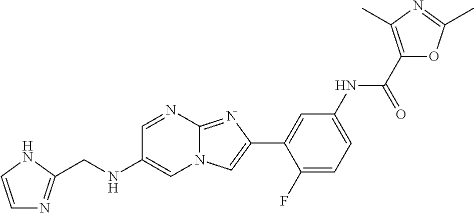 [SMILES: Cc1nc(C)c(C(=O)Nc2ccc(F)c(-c3cn4cc(NCc5ncc[nH]5)cnc4n3)c2)o1] | LCMS m/z = 447.4 (M + 1); RT = 1.15, Method 3. |
| 480 | 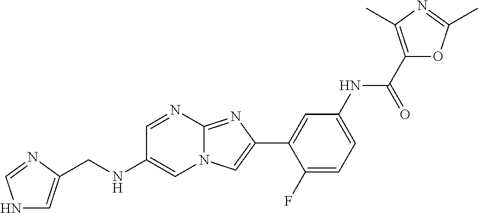 [SMILES: Cc1nc(C)c(C(=O)Nc2ccc(F)c(-c3cn4cc(NCc5c[nH]cn5)cnc4n3)c2)o1] | LCMS m/z = 447.4 (M + 1); RT = 1.18, Method 3. |
| 481 | 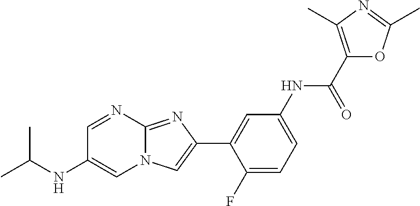 [SMILES: Cc1nc(C)c(C(=O)Nc2ccc(F)c(-c3cn4cc(NC(C)C)cnc4n3)c2)o1] | ¹H NMR (400 MHz, CD₃OD) δ 8.60 (d, J = 2.8 Hz, 1H), 8.36 (dd, J = 6.6, 2.7 Hz, 1H), 8.26 (d, J = 2.3 Hz, 1H), 8.20 (d, J = 2.8 Hz, 1H), 7.70 (ddd, J = 9.1, 4.5, 2.7 Hz, 1H), 7.40 (dd, J = 10.6, 9.0 Hz, 1H), 3.56 (hept, J = 6.4 Hz, 1H), 2.56 (s, 3H), 2.48 (s, 3H), 1.31 (d, J = 6.3 Hz, 6H). LCMS m/z = 409.1 (M + 1); RT = 0.86, Method 2. |
| 482 | 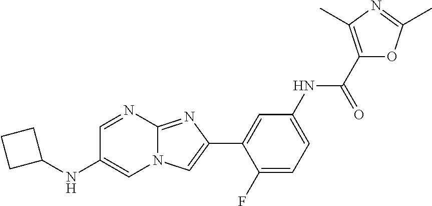 [SMILES: Cc1nc(C)c(C(=O)Nc2ccc(F)c(-c3cn4cc(NC5CCC5)cnc4n3)c2)o1] | ¹H NMR (400 MHz, CD₃OD) δ 8.55 (d, J = 2.8 Hz, 1H), 8.33 (dd, J = 6.7, 2.7 Hz, 1H), 8.24 (d, J = 2.5 Hz, 1H), 8.05 (d, J = 2.9 Hz, 1H), 7.71 (ddd, J = 8.8, 4.3, 2.6 Hz, 1H), 7.37 (dd, J = 10.6, 8.9 Hz, 1H), 3.87 (p, J = 7.4 Hz, 1H), 2.56 (s, 3H), 2.51 (td, J = 6.9, 6.3, 2.6 Hz, 2H), 2.48 (s, 3H), 2.04-1.86 (m, 4H). LCMS m/z = 421.4 (M + 1); RT = 1.52, Method 3. |
| 483 | 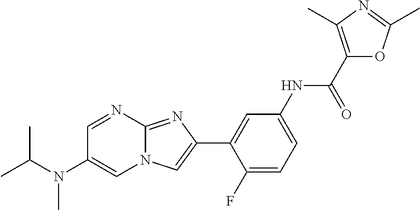 [SMILES: Cc1nc(C)c(C(=O)Nc2ccc(F)c(-c3cn4cc(N(C)C(C)C)cnc4n3)c2)o1] | ¹H NMR (400 MHz, CD₃OD) δ 8.95 (d, J = 2.7 Hz, 1H), 8.39 (d, J = 3.0 Hz, 1H), 8.34 (dd, J = 6.6, 2.7 Hz, 1H), 8.26 (d, J = 2.7 Hz, 1H), 7.73 (ddd, J = 9.0, 4.5, 2.7 Hz, 1H), 7.37 (dd, J = 10.7, 9.0 Hz, 1H), 4.28-4.15 (m, 1H), 2.86 (s, 3H), 2.56 (s, 3H), 2.48 (s, 3H), 1.28 (d, J = 6.6 Hz, 6H). LCMS m/z = 423.3 (M + 1); RT = 2.87, Method 1. |
| 484 | 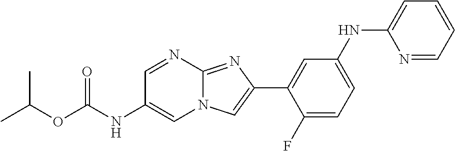 [SMILES: CC(C)OC(=O)Nc1cnc2nc(-c3cc(Nc4ccccn4)ccc3F)cn2c1] | LCMS m/z = 407 (M + 1); RT = 0.72, Method 2. |
| 485 | 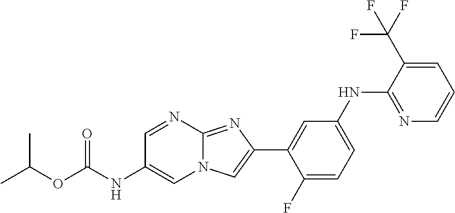 [SMILES: CC(C)OC(=O)Nc1cnc2nc(-c3cc(Nc4ncccc4C(F)(F)F)ccc3F)cn2c1] | LCMS m/z = 475 (M + 1); RT = 1.1, Method 2. |
| 486 | 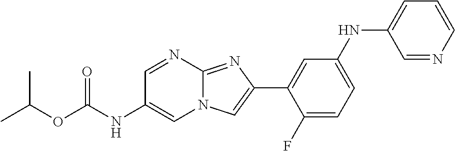 [SMILES: CC(C)OC(=O)Nc1cnc2nc(-c3cc(Nc4cccnc4)ccc3F)cn2c1] | LCMS m/z = 407 (M + 1); RT = 0.7, Method 2. |
| 487 | 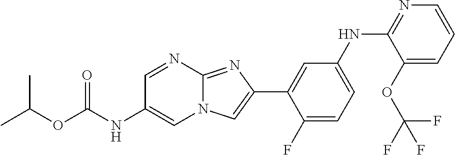 [SMILES: CC(C)OC(=O)Nc1cnc2nc(-c3cc(Nc4ncccc4OC(F)(F)F)ccc3F)cn2c1] | ¹H NMR (400 MHz, CD₃OD) δ 9.27 (s, 1H), 8.46 (d, J = 2.7 Hz, 1H), 8.26 (dd, J = 6.7, 2.8 Hz, 1H), 8.21 (d, J = 3.8 Hz, 1H), 8.06 (dd, J = 5.0, 1.5 Hz, 1H), 7.68 (ddd, J = 8.8, 4.3, 2.8 Hz, 1H), 7.61 (dd, J = 7.8, 1.6 Hz, 1H), 7.41- 7.32 (m, 1H), 7.18 (dd, J = 10.9, 8.9 Hz, 1H), 6.83 (dd, J = 8.0, 4.9 Hz, 1H), 5.02 (p, J = 6.2 Hz, 1H), 1.33 (d, J = 6.3 Hz, 6H). LCMS m/z = 491 (M + 1); RT = 1.14, Method 2. |
| 488 | 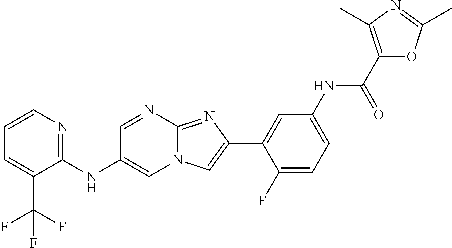 [SMILES: Cc1nc(C)c(C(=O)Nc2ccc(F)c(-c3cn4cc(Nc5ncccc5C(F)(F)F)cnc4n3)c2)o1] | ¹H NMR (400 MHz, DMSO-d₆) δ 10.34 (s, 1H), 9.19 (d, J = 2.6 Hz, 1H), 8.72 (dd, J = 6.9, 2.8 Hz, 1H), 8.68 (d, J = 2.7 Hz, 1H), 8.57 (s, 1H), 8.36 (dd, J = 5.0, 1.7 Hz, 1H), 8.33 (d, J = 4.1 Hz, 1H), 8.03 (dd, J = 7.7, 1.7 Hz, 1H), 7.79 (ddd, J = 8.9, 4.3, 2.7 Hz, 1H), 7.32 (dd, J = 11.0, 8.9 Hz, 1H), 7.00 (dd, J = 7.7, 4.9 Hz, 1H), 2.51 (s, 3H), 2.40 (s, 3H). LCMS m/z = 512.4 (M + 1); RT = 1.72, Method 3. |
| 489 | 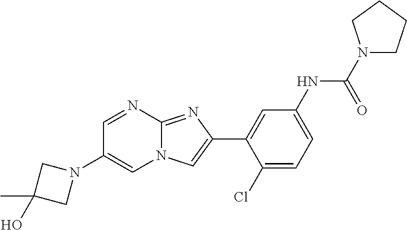 [SMILES: CC1(O)CN(c2cnc3nc(-c4cc(NC(=O)N5CCCC5)ccc4Cl)cn3c2)C1] | ¹H NMR (400 MHz, CD₃OD) δ 8.22 (d, J = 3.4 Hz, 2H), 8.03 (d, J = 2.8 Hz, 1H), 7.90 (d, J = 2.7 Hz, 1H), 7.58 (dd, J = 8.8, 2.6 Hz, 1H), 7.39 (d, J = 8.9 Hz, 1H), 3.93 (d, J = 7.2 Hz, 2H), 3.78 (d, J = 7.3 Hz, 2H), 3.52-3.43 (m, 4H), 2.04-1.92 (m, 4H), 1.60 (s, 3H). LCMS m/z = 427.2 (M + 1); RT = 1.29, Method 3. |
| 490 | 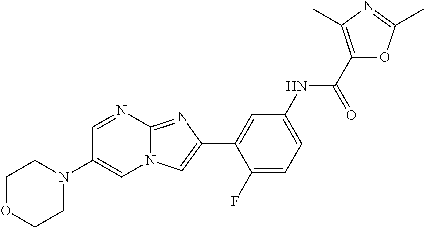 [SMILES: Cc1nc(C)c(C(=O)Nc2ccc(F)c(-c3cn4cc(N5CCOCC5)cnc4n3)c2)o1] | ¹H NMR (400 MHz, CD₃OD) δ 8.62 (d, J = 2.8 Hz, 1H), 8.32 (d, J = 2.9 Hz, 1H), 8.18 (dd, J = 6.7, 2.7 Hz, 1H), 8.10 (d, J = 3.9 Hz, 1H), 7.81 (ddd, J = 8.9, 4.4, 2.8 Hz, 1H), 7.21 (dd, J = 10.9, 8.9 Hz, 1H), 3.94-3.82 (m, 4H), 3.19-3.10 (m, 4H), 2.55 (s, 3H), 2.46 (s, 3H). LCMS m/z = 437 (M + 1); RT = 0.76, Method 2. |
| 491 | 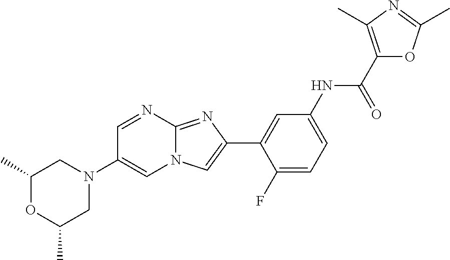 | ¹H NMR (400 MHz, CD₃OD) δ 8.63 (d, J = 2.8 Hz, 1H), 8.31 (d, J = 3.0 Hz, 1H), 8.19 (dd, J = 6.7, 2.7 Hz, 1H), 8.10 (d, J = 3.9 Hz, 1H), 7.81 (ddd, J = 8.9, 4.4, 2.8 Hz, 1H), 7.21 (dd, J = 10.9, 9.0 Hz, 1H), 3.86 (ddd, J = 10.2, 6.2, 2.3 Hz, 2H), 3.48 (dt, J = 11.0, 2.0 Hz, 2H), 2.56 (s, 3H), 2.46 (s, 3H), 2.39 (dd, J = 11.7, 10.2 Hz, 2H), 1.25 (d, J = 6.3 Hz, 6H). LCMS m/z = 465.1 (M + 1); RT = 0.92, Method 2. |
| 492 | 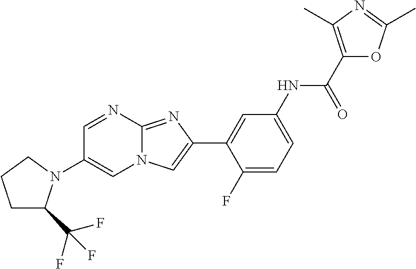 [SMILES: Cc1nc(C)c(C(=O)Nc2ccc(F)c(-c3cn4cc(N5CCC[C@@H]5C(F)(F)F)cnc4n3)c2)o1] | ¹H NMR (400 MHz, CD₃OD) δ 8.58 (d, J = 2.9 Hz, 1H), 8.32 (d, J = 3.0 Hz, 1H), 8.20 (dd, J = 6.6, 2.7 Hz, 1H), 8.13 (d, J = 3.9 Hz, 1H), 7.93 (dd, J = 8.1, 1.6 Hz, 1H), 7.82 (ddd, J = 8.8, 4.4, 2.8 Hz, 1H), 7.23 (dd, J = 11.0, 8.9 Hz, 1H), 4.67-4.53 (m, 1H), 3.72 (dd, J = 9.2, 6.0 Hz, 1H), 2.56 (s, 3H), 2.47 (s, 3H), 2.22 (dq, J = 24.8, 9.9, 8.4 Hz, 4H). LCMS m/z = 489 (M + 1); RT = 1.03, Method 2. |
| 493 | 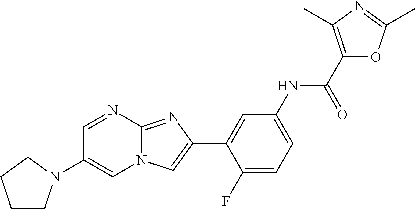 [SMILES: Cc1nc(C)c(C(=O)Nc2ccc(F)c(-c3cn4cc(N5CCCC5)cnc4n3)c2)o1] | ¹H NMR (400 MHz, DMSO-d₆) δ 10.30 (s, 1H), 8.68 (dd, J = 6.9, 2.8 Hz, 1H), 8.45 (d, J = 3.0 Hz, 1H), 8.15 (d, J = 3.0 Hz, 1H), 8.11 (d, J = 4.3 Hz, 1H), 7.73 (ddd, J = 8.7, 4.5, 2.8 Hz, 1H), 7.28 (dd, J = 11.0, 8.9 Hz, 1H), 3.27 (dq, J = 4.3, 2.6, 1.9 Hz, 4H), 2.50 (s, 3H), 2.40 (s, 3H), 2.02-1.94 (m, 4H). LCMS m/z = 421.4 (M + 1); RT = 1.46, Method 3. |
| 494 | 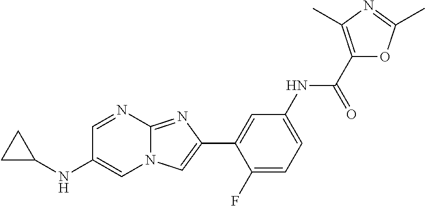 [SMILES: Cc1nc(C)c(C(=O)Nc2ccc(F)c(-c3cn4cc(NC5CC5)cnc4n3)c2)o1] | 1H NMR (400 MHz, CD₃OD) δ 8.24 (d, J = 2.8 Hz, 1H), 8.21 (d, J = 2.7 Hz, 1H), 8.16 (dd, J = 6.8, 2.8 Hz, 1H), 8.10 (d, J = 4.2 Hz, 1H), 7.81 (ddd, J = 8.9, 4.4, 2.8 Hz, 1H), 7.22 (dd, J = 11.0, 8.9 Hz, 1H), 2.56 (s, 3H), 2.46 (s, 3H), 2.40 (tt, J = 6.8, 3.5 Hz, 1H), 0.81 (td, J = 6.6, 4.6 Hz, 2H), 0.58-0.49 (m, 2H). LCMS m/z = 407.4 (M + 1); RT = 1.46, Method 3. |
| 495 | 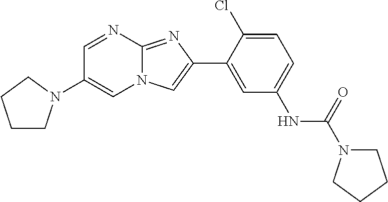 [SMILES: O=C(Nc1ccc(Cl)c(-c2cn3cc(N4CCCC4)cnc3n2)c1)N1CCCC1] | ¹H NMR (600 MHz, CD₃OD) δ 8.42 (d, J = 2.9 Hz, 1H), 8.21 (s, 1H), 7.98 (d, J = 2.9 Hz, 1H), 7.89 (d, J = 2.7 Hz, 1H), 7.58 (dd, J = 8.7, 2.7 Hz, 1H), 7.38 (d, J = 8.7 Hz, 1H), 3.51-3.45 (m, 4H), 3.35 (d, J = 3.0 Hz, 4H), 2.10-2.06 (m, 4H), 2.01-1.95 (m, 4H). LCMS m/z = 411 (M + 1); RT = 0.8, Method 2. |
| 496 | 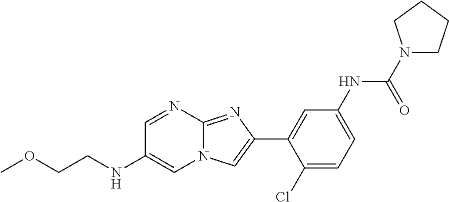 [SMILES: COCCNc1cnc2nc(-c3cc(NC(=O)N4CCCC4)ccc3Cl)cn2c1] | ¹H NMR (400 MHz, CD₃OD) δ 8.33 (d, J = 2.7 Hz, 1H), 8.19 (s, 1H), 8.02 (d, J = 2.8 Hz, 1H), 7.88 (d, J = 2.7 Hz, 1H), 7.58 (dd, J = 8.8, 2.7 Hz, 1H), 7.39 (d, J = 8.7 Hz, 1H), 3.67 (t, J = 5.2 Hz, 2H), 3.48 (d, J = 6.6 Hz, 4H), 3.42 (s, 3H), 3.25 (t, J = 5.2 Hz, 2H), 2.02-1.94 (m, 4H). LCMS m/z = 415 (M + 1); RT = 0.69, Method 2. |
| 497 | 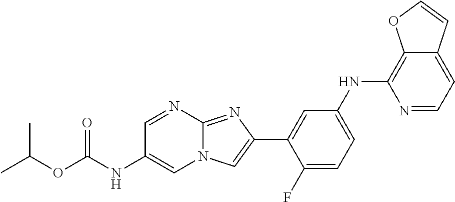 [SMILES: CC(C)OC(=O)Nc1cnc2nc(-c3cc(Nc4nccc5ccoc45)ccc3F)cn2c1] | ¹H NMR (400 MHz, CD₃OD) δ 9.24 (s, 1H), 8.44 (d, J = 2.7 Hz, 1H), 8.25-8.15 (m, 2H), 7.93 (d, J = 2.1 Hz, 1H), 7.89- 7.78 (m, 2H), 7.17 (dd, J = 11.0, 8.9 Hz, 1H), 7.09 (d, J = 5.5 Hz, 1H), 6.89 (d, J = 2.0 Hz, 1H), 5.01 (p, J = 6.3 Hz, 1H), 1.32 (d, J = 6.3 Hz, 6H). LCMS m/z = 447.2 (M + 1); RT = 2.62, Method 1. |
| 498 | 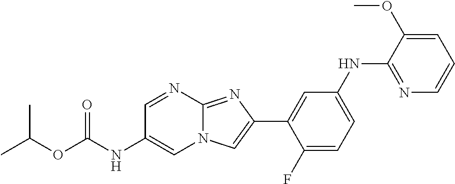 [SMILES: COc1cccnc1Nc1ccc(F)c(-c2cn3cc(NC(=O)OC(C)C)cnc3n2)c1] | 1H NMR (400 MHz, DMSO-d₆) δ 9.99 (s, 1H), 9.29 (s, 1H), 8.65 (dd, J = 6.8, 2.9 Hz, 1H), 8.48 (d, J = 2.7 Hz, 1H), 8.42-8.30 (m, 2H), 7.87 (ddd, J = 8.8, 4.4, 2.8 Hz, 1H), 7.75 (dd, J = 5.1, 1.4 Hz, 1H), 7.25-7.15 (m, 2H), 6.77 (dd, J = 7.8, 5.0 Hz, 1H), 4.94 (p, J = 6.2 Hz, 1H), 3.89 (s, 3H), 1.29 (d, J = 6.2 Hz, 6H). LCMS m/z = 437.2 (M + 1); RT = 1.33, Method 3. |
| 499 | 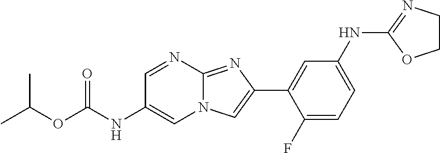 [SMILES: CC(C)OC(=O)Nc1cnc2nc(-c3cc(NC4=NCCO4)ccc3F)cn2c1] | ¹H NMR (400 MHz, DMSO-d₆) δ 9.92 (s, 1H), 9.21 (s, 1H), 8.41 (d, J = 2.7, 1H), 8.26 (d, J = 4.2, 1H), 7.75 (s, 1H), 7.27 (t, J = 7.3, 1H), 7.21-7.01 (m, 2H), 4.96-4.79 (m, 1H), 4.28 (s, 1H), 4.10 (s, 1H), 3.67 (s, 1H), 1.23 (s, 3H), 1.21 (d, J = 2.5, 3H), 1.13 (d, J = 6.1, 3H). LCMS m/z = 399 (M + 1); RT = 0.67, Method 2. |
| 500 | 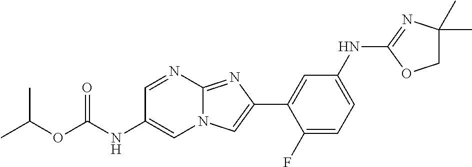 [SMILES: CC(C)OC(=O)Nc1cnc2nc(-c3cc(NC4=NC(C)(C)CO4)ccc3F)cn2c1] | LCMS m/z = 427 (M + 1); RT = 0.71, Method 2. |
| 501 | 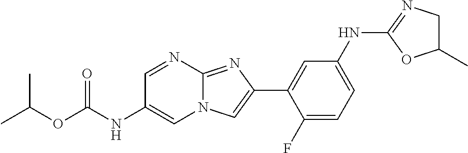 [SMILES: CC(C)OC(=O)Nc1cnc2nc(-c3cc(NC4=NCC(C)O4)ccc3F)cn2c1] | LCMS m/z = 413 (M + 1); RT = 0.7, Method 2. |
| 502 | 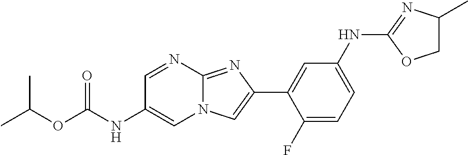 [SMILES: CC1COC(Nc2ccc(F)c(-c3cn4cc(NC(=O)OC(C)C)cnc4n3)c2)=N1] | LCMS m/z = 413 (M + 1); RT = 0.69, Method 2. |
| 503 | 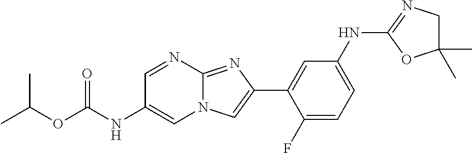 [SMILES: CC(C)OC(=O)Nc1cnc2nc(-c3cc(NC4=NCC(C)(C)O4)ccc3F)cn2c1] | LCMS m/z = 427 (M + 1); RT = 0.73, Method 2. |
| 504 | 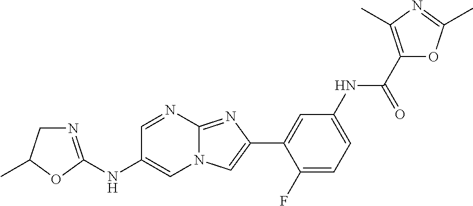 | LCMS m/z = 450.4 (M + 1); RT = 1.31, Method 3. |
| 505 | 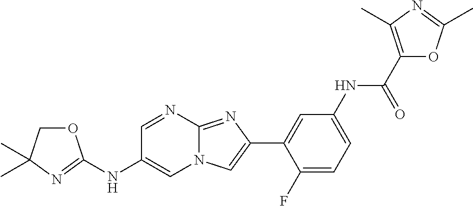 | LCMS m/z = 464.4 (M + 1); RT = 1.33, Method 3. |
| 506 | 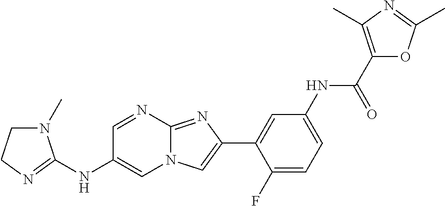 [SMILES: Cc1nc(C)c(C(=O)Nc2ccc(F)c(-c3cn4cc(NC5=NCCN5C)cnc4n3)c2)o1] | LCMS m/z = 449 (M + 1); RT = 0.59, Method 2. |
| 507 | 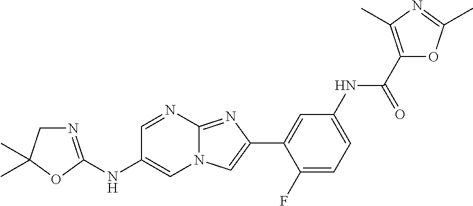 | LCMS m/z = 464.4 (M + 1); RT = 1.37, Method 3. |
| 508 | 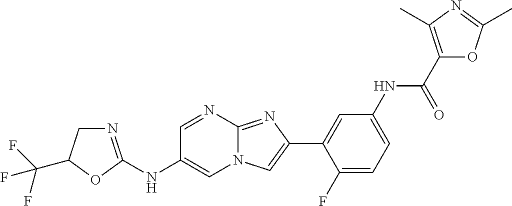 [SMILES: Cc1nc(C)c(C(=O)Nc2ccc(F)c(-c3cn4cc(NC5=NCC(C(F)(F)F)O5)cnc4n3)c2)o1] | LCMS m/z = 504.4 (M + 1); RT = 1.48, Method 3. |
| 509 | 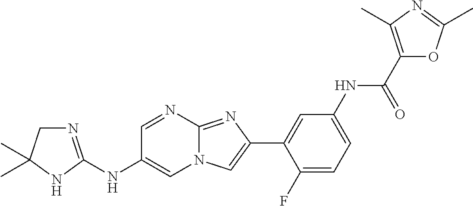 | LCMS m/z = 463.4 (M + 1); RT = 1.33, Method 3. |
| 510 | 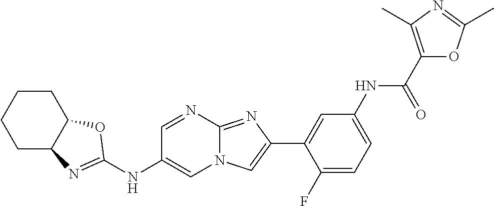 [SMILES: Cc1nc(C)c(C(=O)Nc2ccc(F)c(-c3cn4cc(NC5=N[C@H]6CCCC[C@@H]6O5)cnc4n3)c2)o1] | LCMS m/z = 490.4 (M + 1); RT = 1.44, Method 3. |
| 511 | 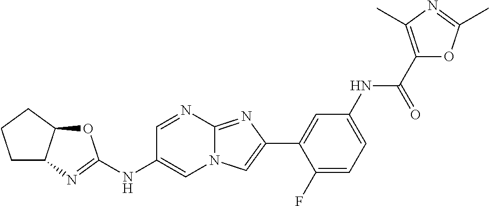 [SMILES: Cc1nc(C)c(C(=O)Nc2ccc(F)c(-c3cn4cc(NC5=N[C@@H]6CCC[C@H]6O5)cnc4n3)c2)o1] | LCMS m/z = 476.4 (M + 1); RT = 1.39, Method 3. |
| 512 | 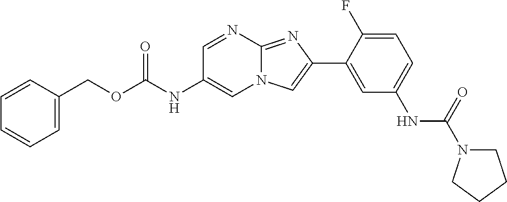 [SMILES: O=C(Nc1cnc2nc(-c3cc(NC(=O)N4CCCC4)ccc3F)cn2c1)OCc1ccccc1] | ¹H NMR (400 MHz, DMSO-d₆) δ 10.18 (brs, 1H), 9.28 (brs, 1H), 8.49 (d, J = 2.69 Hz, 1H) 8.30-8.39 (m, 3H), 7.63 (ddd, J = 8.93, 4.46, 2.87 Hz, 1H), 7.33-7.49 (m, 5H), 7.18 (dd, J = 10.94, 8.99 Hz, 1H), 5.21 (s, 2H), 3.39 (t, J = 6.66 Hz, 4H), 1.82-1.90 (m, 4H). LCMS m/z = 475.4 (M + 1); RT = 1.63, Method 3. |
| 513 | 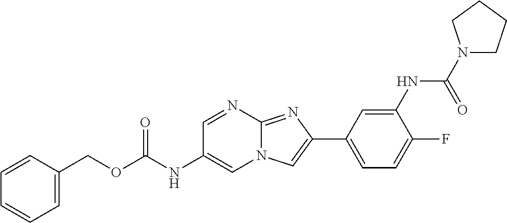 [SMILES: O=C(Nc1cnc2nc(-c3ccc(F)c(NC(=O)N4CCCC4)c3)cn2c1)OCc1ccccc1] | LCMS m/z = 475.4 (M + 1); RT = 1.63, Method 3. |
| 514 | 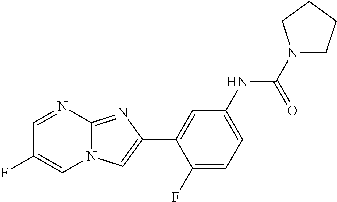 [SMILES: O=C(Nc1ccc(F)c(-c2cn3cc(F)cnc3n2)c1)N1CCCC1] | LCMS m/z = 344.3 (M + 1); RT = 1.29, Method 3. |
| 515 | 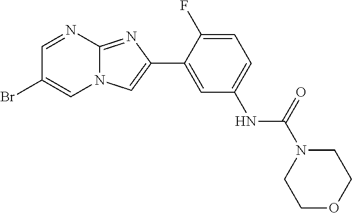 [SMILES: O=C(Nc1ccc(F)c(-c2cn3cc(Br)cnc3n2)c1)N1CCOCC1] | LCMS m/z = 420.2 (M + 1), 422.2 (M + 3). RT = 1.35, Method 3. |
| 516 | 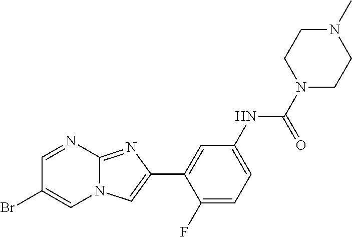 [SMILES: CN1CCN(C(=O)Nc2ccc(F)c(-c3cn4cc(Br)cnc4n3)c2)CC1] | LCMS m/z = 433.2 (M + 1), 435.2 (M + 3). RT = 1.17, Method 3. |
| 517 | 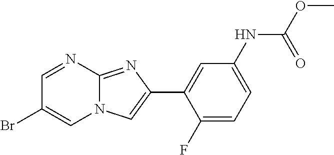 [SMILES: COC(=O)Nc1ccc(F)c(-c2cn3cc(Br)cnc3n2)c1] | LCMS m/z = 365.1 (M + 1), 367.1 (M + 3). RT = 1.5, Method 3. |
| 518 | 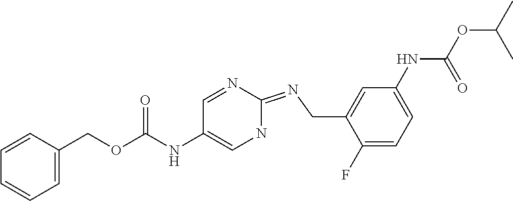 | LCMS m/z = 464.4 (M + 1); RT = 1.79, Method 3. |
| 519 | 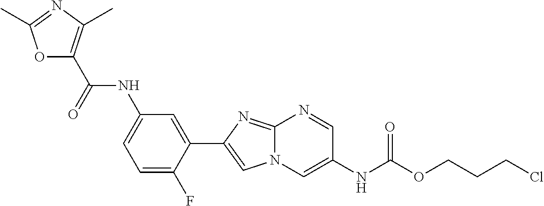 [SMILES: Cc1nc(C)c(C(=O)Nc2ccc(F)c(-c3cn4cc(NC(=O)OCCCCl)cnc4n3)c2)o1] | LCMS m/z = 487.3 (M + 1); RT = 1.63, Method 3. |
| 520 | 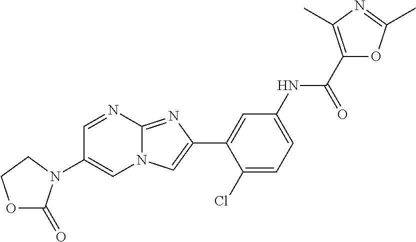 [SMILES: Cc1nc(C)c(C(=O)Nc2ccc(Cl)c(-c3cn4cc(N5CCOC5=O)cnc4n3)c2)o1] | ¹H NMR (400 MHz, DMSO-d₆) δ 10.42 (s, 1H), 9.28 (d, J = 2.8 Hz, 1H), 8.93 (d, J = 2.8 Hz, 1H), 8.76 (d, J = 2.7 Hz, 1H), 8.68 (s, 1H), 7.84 (dd, J = 2.7 Hz, 8.8, 1H), 7.53 (d, J = 8.7 Hz, 1H), 4.54 (dd, J = 6.9, 9.0 Hz, 2H), 4.15 (dd, J = 7.2, 8.9 hz, 2H), 2.50 (s, 3H), 2.40 (s, 3H). LCMS m/z = 452.9 (M + 1); RT = 0.78, Method 2. |
| 521 | 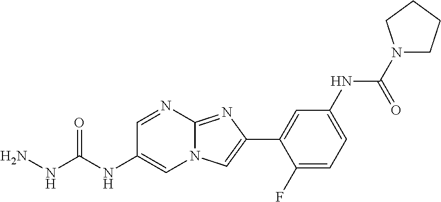 [SMILES: NNC(=O)Nc1cnc2nc(-c3cc(NC(=O)N4CCCC4)ccc3F)cn2c1] | LCMS m/z = 399.0 (M + 1); RT = 0.59, Method 2. |
| 522 | 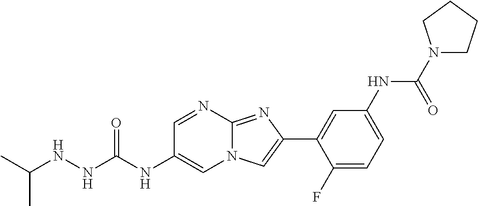 [SMILES: CC(C)NNC(=O)Nc1cnc2nc(-c3cc(NC(=O)N4CCCC4)ccc3F)cn2c1] | ¹H NMR: 9.50 (d, J = 2.4 Hz, 1H), 8.38 (d, J = 2.7 Hz, 1H), 8.04 (dd, J = 6.6, 2.7 Hz, 1H), 7.51 (dq, J = 7.2, 2.8, 2.2 Hz, 1H), 7.26 (dd, J = 10.6, 9.1 Hz, 2H), 3.56-3.45 (m, 5H), 2.00 (d, J = 4.4 Hz, 4H), 1.21-1.11 (m, 6H). |
| 523 | 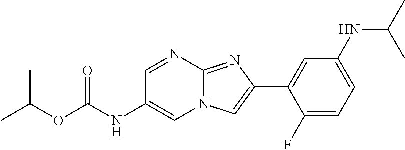 [SMILES: CC(C)Nc1ccc(F)c(-c2cn3cc(NC(=O)OC(C)C)cnc3n2)c1] | LCMS m/z = 372.3 (M + 1); RT = 1.33, Method 3. |
| 524 | 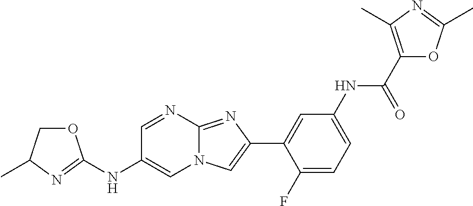 | LCMS m/z = 450.4 (M + 1); RT = 1.31, Method 3. |
| 525 | 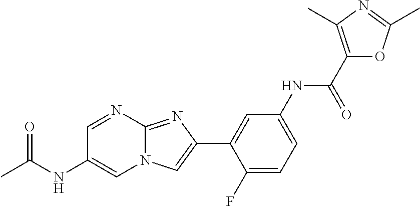 [SMILES: CC(=O)Nc1cnc2nc(-c3cc(NC(=O)c4oc(C)nc4C)ccc3F)cn2c1] | ¹H NMR (400 MHz, DMSO-d₆) δ 10.67 (s, 1H), 10.34 (s, 1H), 9.65 (d, J = 2.6 Hz, 1H), 8.78 (d, J = 2.6 Hz, 1H), 8.71 (dd, J = 2.8, 6.9 Hz, 1H), 8.42 (d, J = 4.2 Hz, 1H), 7.79 (ddd, J = 2.8, 4.4, 8.8 Hz, 1H), 7.32 (dd, J = 9.0, 10.9 Hz, 1H), 2.53 (s, 5H), 2.41 (d, J = 7.5 Hz, 6H). LCMS m/z = 409.4 (M + 1); RT = 1.37, Method 3. |
| 526 | 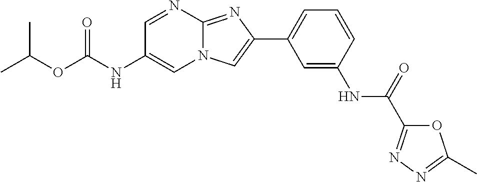 [SMILES: Cc1nnc(C(=O)Nc2cccc(-c3cn4cc(NC(=O)OC(C)C)cnc4n3)c2)o1] | LCMS m/z = 422 (M + 1); RT = 0.77, Method 2. |
| 527 | 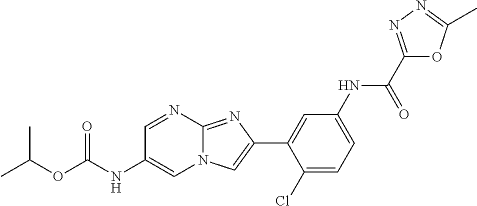 [SMILES: Cc1nnc(C(=O)Nc2ccc(Cl)c(-c3cn4cc(NC(=O)OC(C)C)cnc4n3)c2)o1] | 1H NMR (400 MHz, CD₃OD) δ 9.55 (s, 1H), 8.74 (d, J = 2.6 Hz, 1H), 8.52 (s, 1H), 8.33 (d, J = 2.6 Hz, 1H), 7.88 (dd, J = 8.8, 2.6 Hz, 1H), 7.66 (d, J = 8.8 Hz, 1H), 5.05 (hept, J = 5.3 Hz, 1H), 2.67 (s, 3H), 1.35 (d, J = 6.2 Hz, 6H). LCMS m/z = 456 (M + 1); RT = 0.91, Method 2. |
| 528 | 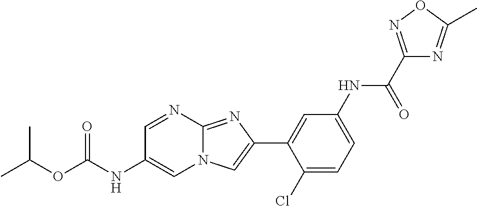 [SMILES: Cc1nc(C(=O)Nc2ccc(Cl)c(-c3cn4cc(NC(=O)OC(C)C)cnc4n3)c2)no1] | LCMS m/z = 456 (M + 1); RT = 0.94, Method 2. |
| 529 | 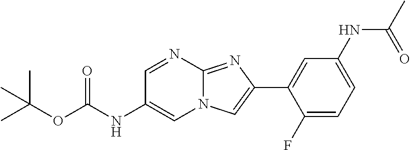 [SMILES: CC(=O)Nc1ccc(F)c(-c2cn3cc(NC(=O)OC(C)(C)C)cnc3n2)c1] | ¹H NMR (400 MHz, DMSO-d₆) δ 10.10 (s, 1H), 9.78 (brs, 1H), 9.30 (brs, 1H), 8.48 (d, J = 2.69 Hz, 1H), 8.41 (dd, J = 6.85, 2.81 Hz, 1H), 8.33 (d, J = 4.16 Hz, 1H), 7.66 (ddd, J = 8.83, 4.43, 2.87 Hz, 1H), 7.24 (dd, J = 11.00, 9.05 Hz, 1H), 2.06 (s, 3H), 1.51 (s, 9H). LCMS m/z = 386.3 (M + 1); RT = 1.48, Method 3. |
| 530 | 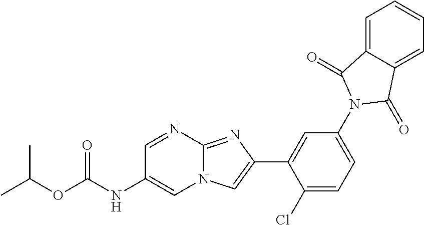 [SMILES: CC(C)OC(=O)Nc1cnc2nc(-c3cc(N4C(=O)c5ccccc5C4=O)ccc3Cl)cn2c1] | LCMS m/z = 476.1 (M + 1); RT = 3.28, Method 1. |
| 531 | 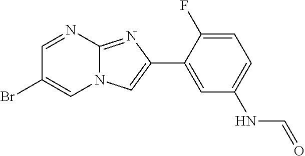 [SMILES: O=CNc1ccc(F)c(-c2cn3cc(Br)cnc3n2)c1] | ¹H NMR (400 MHz, DMSO-d₆) δ 10.37 (s, 1H), 9.35 (d, J = 2.57 Hz, 1H), 8.65 (d, J = 2.57 Hz, 1H), 8.51 (dd, J = 6.79, 2.75 Hz, 1H), 8.31 (d, J = 1.83 Hz, 1H), 8.23-8.27 (m, 1H), 7.68 (ddd, J = 8.83, 4.49, 2.81 Hz, 1H), 7.32 (dd, J = 11.13, 8.93 Hz, 1H). LCMS m/z = 335.1 (M + 1), 337.1 (M + 3). RT = 0.78, Method 3. |
| 532 | 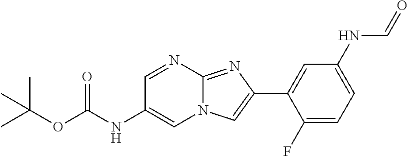 [SMILES: CC(C)(C)OC(=O)Nc1cnc2nc(-c3cc(NC=O)ccc3F)cn2c1] | ¹H NMR (400 MHz, DMSO-d₆) δ 10.34 (s, 1H), 9.79 (brs, 1H), 9.30 (brs, 1H), 8.48 (d, J = 2.69 Hz, 1H), 8.45 (dd, J = 6.72, 2.69 Hz, 1H), 8.34-8.38 (m, 1H), 8.30 (d, J = 1.71 Hz, 1H), 7.65 (ddd, J = 8.83, 4.37, 2.93 Hz, 1H), 7.28 (dd, J = 11.07, 8.86 Hz, 1H), 1.51 (s, 9H). LCMS m/z = 372.3 (M + 1); RT = 1.48, Method 3. |
| 533 | 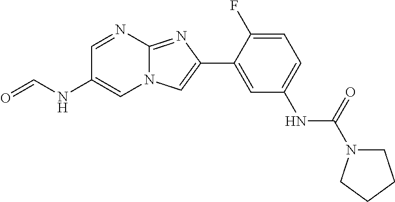 [SMILES: O=CNc1cnc2nc(-c3cc(NC(=O)N4CCCC4)ccc3F)cn2c1] | ¹H NMR (400 MHz, CD₃OD) δ 9.57 (d, J = 2.57 Hz, 1H), 8.55 (s, 1H), 8.53 (d, J = 2.57 Hz, 1H), 8.43 (s, 1H), 8.23 (d, J = 3.91 Hz, 1H), 7.99 (dd, J = 6.72, 2.69 Hz, 1H), 7.58 (ddd, J = 8.93, 4.52, 2.81 Hz, 1H), 7.16 (dd, J = 11.00, 8.93 Hz, 1H), 3.47-3.52 (m, 4H), 2.00 (s, 4H). |
| 534 | 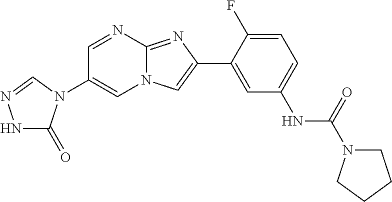 [SMILES: O=C(Nc1ccc(F)c(-c2cn3cc(-n4cn[nH]c4=O)cnc3n2)c1)N1CCCC1] | ¹H NMR (400 MHz, CD₃OD) δ 9.38 (d, J = 2.6 Hz, 1H), 8.88 (d, J = 2.6 Hz, 1H), 8.35 (d, J = 3.6 Hz, 1H), 8.25 (s, 1H), 8.06 (dd, J = 6.6, 2.8 Hz, 1H), 7.60 (ddd, J = 6.9, 4.4, 2.8 Hz, 1H), 7.20 (dd, J = 10.9, 9.0 Hz, 1H), 3.56- 3.43 (m, 4H), 2.05-1.94 (m, 4H). LCMS m/z = 409 (M + 1); RT = 0.65. Method 2. |
| 535 | 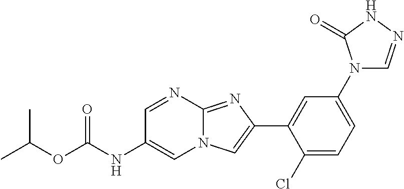 [SMILES: CC(C)OC(=O)Nc1cnc2nc(-c3cc(-n4cn[nH]c4=O)ccc3Cl)cn2c1] | LCMS m/z = 414 (M + 1); RT = 0.81, Method 2. |
| 536 | 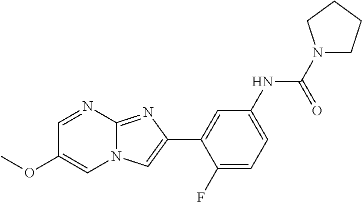 [SMILES: COc1cnc2nc(-c3cc(NC(=O)N4CCCC4)ccc3F)cn2c1] | ¹H NMR (400 MHz, CD₃OD) δ 8.55 (d, J = 3.1 Hz, 1H), 8.42 (d, J = 2.9 Hz, 1H), 8.13 (d, J = 3.8 Hz, 1H), 7.93 (dd, J = 6.7, 2.7 Hz, 1H), 7.56 (ddd, J = 8.9, 4.4, 2.8 Hz, 1H), 7.14 (dd, J = 11.0, 8.9 Hz, 1H), 3.91 (s, 3H), 3.55-3.43 (m, 4H), 2.04-1.93 (m, 4H). LCMS m/z = 356.1 (M + 1); RT = 0.72, Method 2. |
| 537 | 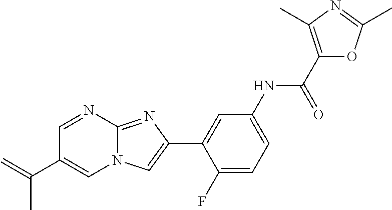 [SMILES: C=C(C)c1cnc2nc(-c3cc(NC(=O)c4oc(C)nc4C)ccc3F)cn2c1] | ¹H NMR (400 MHz, CD₃OD) δ 8.94 (d, J = 2.5 Hz, 1H), 8.88 (d, J = 2.5 Hz, 1H), 8.30-8.19 (m, 2H), 7.84 (ddd, J = 8.9, 4.4, 2.8 Hz, 1H), 7.24 (dd, J = 10.9, 8.9 Hz, 1H), 5.67 (s, 1H), 5.32 (d, J = 1.8 Hz, 1H), 2.56 (s, 3H), 2.47 (s, 3H), 2.23 (s, 3H). LCMS m/z = 392.3 (M + 1); RT = 1.63, Method 3. |
| 538 | 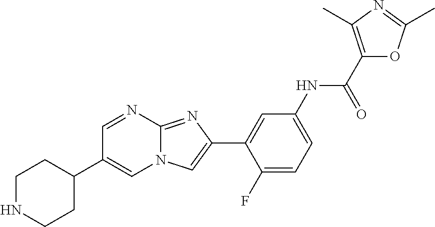 [SMILES: Cc1nc(C)c(C(=O)Nc2ccc(F)c(-c3cn4cc(C5CCNCC5)cnc4n3)c2)o1] | LCMS m/z = 435.1 (M + 1); RT = 0.58, Method 2. |
| 539 | 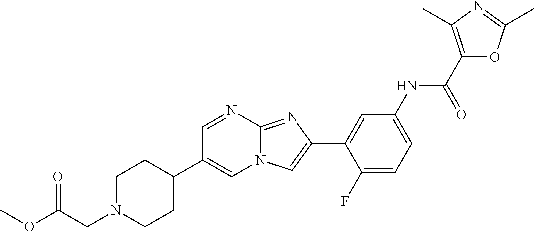 | ¹H NMR (400 MHz, DMSO-d₆) δ 10.36 (s, 1H), 8.93 (s, 1H), 8.72 (dd, J = 7.0, 2.6 Hz, 1H), 8.32 (d, J = 3.9 Hz, 1H), 7.78 (ddd, J = 9.0, 4.5, 2.8 Hz, 1H), 7.35 (dd, J = 10.9, 8.9 Hz, 1H), 4.31 (s, 2H), 3.80 (s, 3H), 3.74-3.58 (m, 2H), 3.34-3.17 (m, 2H), 2.99 (d, J = 12.9 Hz, 1H), 2.50 (s, 3H), 2.40 (s, 3H), 2.27-2.12 (m, 2H), 2.10-1.96 (m, 2H). LCMS m/z = 507.1 (M + 1); RT = 0.61, Method 2. |
| 540 | 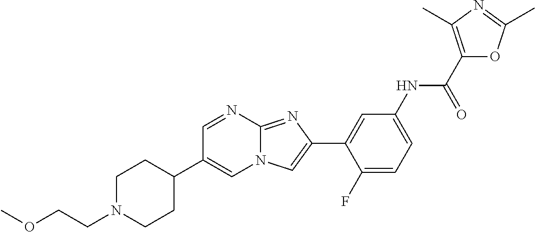 | LCMS m/z = 493.1 (M + 1); RT = 0.61, Method 2. |
| 541 | 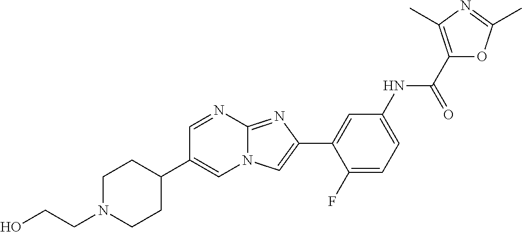 [SMILES: Cc1nc(C)c(C(=O)Nc2ccc(F)c(-c3cn4cc(C5CCN(CCO)CC5)cnc4n3)c2)o1] | ¹H NMR (400 MHz, DMSO-d₆) δ 10.35 (s, 1H), 9.42 (s, 1H), 8.90 (d, J = 2.6 Hz, 1H), 8.83-8.65 (m, 1H), 8.62 (t, J = 1.9 Hz, 1H), 8.32 (dd, J = 4.0, 1.5 Hz, 1H), 7.78 (ddd, J = 9.0, 4.5, 2.8 Hz, 1H), 7.34 (dd, J = 11.0, 8.9 Hz, 1H), 3.94-3.71 (m, 2H), 3.67 (d, J = 12.0 Hz, 2H), 3.45-3.29 (m, 1H), 3.18 (ddt, J = 34.4, 13.6, 7.8 Hz, 2H), 2.99 (td, J = 10.4, 8.5, 6.2 Hz, 1H), 2.50 (s, 3H), 2.40 (s, 3H), 2.25-1.87 (m, 4H). LCMS m/z = 479.1 (M + 1); RT = 0.57, Method 2. |
| 542 | 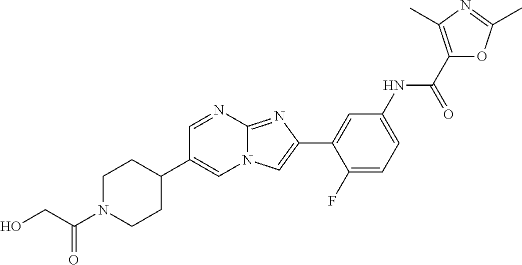 [SMILES: Cc1nc(C)c(C(=O)Nc2ccc(F)c(-c3cn4cc(C5CCN(C(=O)CO)CC5)cnc4n3)c2)o1] | ¹H NMR (400 MHz, DMSO-d₆) δ 10.35 (s, 1H), 8.93 (dd, J = 2.7, 0.9 Hz, 1H), 8.83-8.58 (m, 2H), 8.27 (d, J = 3.9 Hz, 1H), 7.78 (ddd, J = 9.0, 4.5, 2.8 Hz, 1H), 7.35 (dd, J = 10.9, 8.9 Hz, 1H), 4.66-4.41 (m, 1H), 4.14 (d, J = 5.8 Hz, 2H), 3.84 (d, J = 12.8 Hz, 1H), 3.11 (t, J = 13.1 Hz, 1H), 3.03-2.92 (m, 1H), 2.74 (dd, J = 14.0, 11.3 Hz, 1H), 2.50 (dd, J = 3.6, 1.8 Hz, 3H), 2.40 (s, 3H), 2.03-1.87 (m, 2H), 1.60 (ddq, J = 28.8, 12.4, 6.2, 4.2 Hz, 2H). LCMS m/z = 493.1 (M + 1); RT = 0.7, Method 2. |
| 543 | 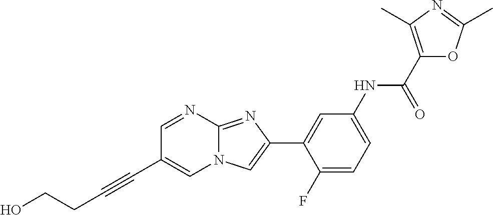 [SMILES: Cc1nc(C)c(C(=O)Nc2ccc(F)c(-c3cn4cc(C#CCCO)cnc4n3)c2)o1] | ¹H NMR (400 MHz, DMSO-d₆) δ 10.16 (s, 1H), 8.97 (d, J = 2.4 Hz, 1H), 8.53 (dd, J = 6.9, 2.8 Hz, 1H), 8.37 (d, J = 2.4 Hz, 1H), 8.06 (d, J = 4.1 Hz, 1H), 7.63 (ddd, J = 8.9, 4.5, 2.8 Hz, 1H), 7.15 (dd, J = 11.1, 8.9 Hz, 1H), 4.80 (t, J = 5.6 Hz, 2H), 3.44 (td, J = 6.7, 5.5 Hz, 2H), 2.44 (s, 3H), 2.31 (s, 3H). LCMS m/z = 420 (M + 1); RT = 0.78, Method 4. |
| 544 | 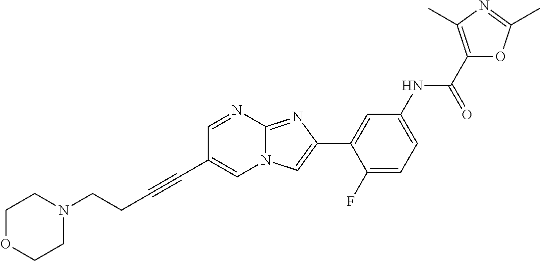 [SMILES: Cc1nc(C)c(C(=O)Nc2ccc(F)c(-c3cn4cc(C#CCCN5CCOCC5)cnc4n3)c2)o1] | LCMS m/z = 489.1 (M + 1); RT = 0.6, Method 4. |
| 545 | 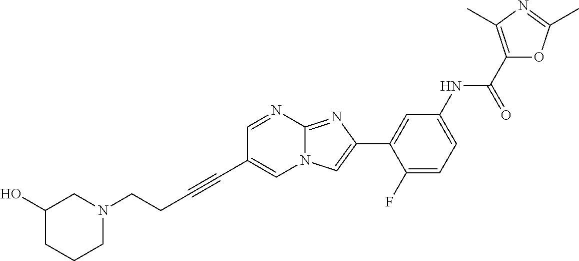 [SMILES: Cc1nc(C)c(C(=O)Nc2ccc(F)c(-c3cn4cc(C#CCCN5CCCC(O)C5)cnc4n3)c2)o1] | LCMS m/z = 503.1 (M + 1); RT = 0.6, Method 4. |
| 546 | 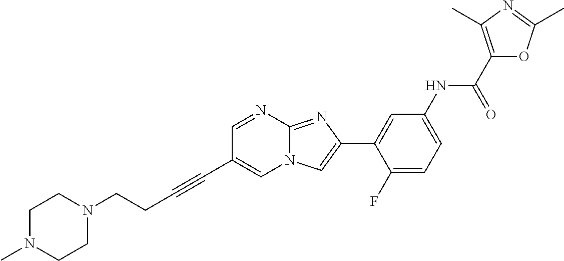 | LCMS m/z = 502.1 (M + 1); RT = 0.53, Method 4. |
| 547 | 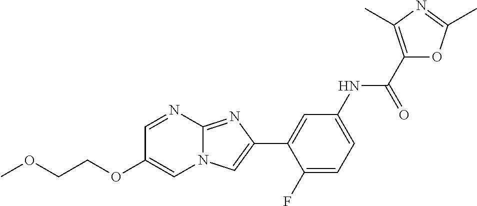 | LCMS m/z = 426.1 (M + 1); RT = 0.77, Method 4. |
| 548 | 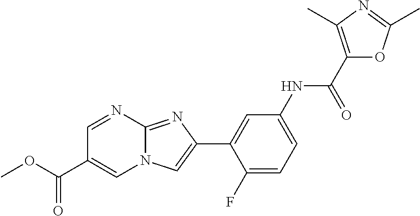 | ¹H NMR (400 MHz, DMSO-d₆) δ 10.37 (s, 1 H), 9.71 (d, J = 2.4 Hz, 1H), 8.95 (d, J = 2.4 Hz, 1H), 8.75 (dd, J = 6.8, 2.8 Hz, 1H), 7.79-7.64 (m, 1H), 7.36 (dd, J = 11.0, 8.9 Hz, 1H), 3.94 (s, 3H), 2.51 (s, 3H), 2.41 (s, 3H). LCMS m/z = 410.2 (M + 1); RT = 1.54, Method 3. |
| 549 | 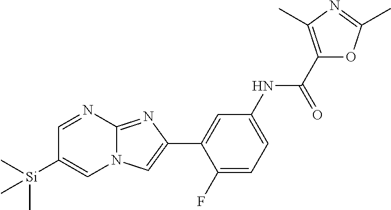 | ¹H NMR (400 MHz, DMSO-d₆) δ 10.31 (s, 1H), 9.02 (d, J = 2.01 Hz, 1H), 8.73 (dd, J = 6.90, 2.64 Hz, 1H), 8.63 (d, J = 2.01 Hz, 1H), 8.26 (d, J = 4.27 Hz, 1H), 7.78 (dd, J = 7.53, 4.52 Hz, 1H), 7.28- 7.37 (m, 1H), 2.48 (s, 3H), 2.40 (s, 3H), 0.36 (s, 9H). LCMS m/z = 424 (M + 1); RT = 1.04, Method 2. |
| 550 | 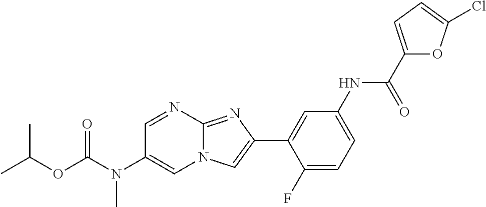 [SMILES: CC(C)OC(=O)N(C)c1cnc2nc(-c3cc(NC(=O)c4ccc(Cl)o4)ccc3F)cn2c1] | ¹H NMR (400 MHz, CD₃OD) δ 9.08 (d, J = 2.6 Hz, 1H), 8.83 (s, 1H), 8.31 (dt, J = 4.7, 2.1 Hz, 2H), 7.81 (ddd, J = 9.1, 4.4, 2.7 Hz, 1H), 7.34 (d, J = 3.5 Hz, 1H), 7.30 (dd, J = 10.8, 9.1 Hz, 1H), 6.57 (d, J = 3.5 Hz, 1H), 5.00 (hept, J = 6.0 Hz, 1H), 3.40 (s, 3H), 1.30 (d, J = 6.4 Hz, 6H). LCMS m/z = 472 (M + 1); RT = 1.02, Method 2. |
| 551 | 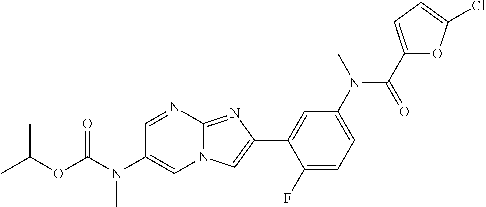 [SMILES: CC(C)OC(=O)N(C)c1cnc2nc(-c3cc(N(C)C(=O)c4ccc(Cl)o4)ccc3F)cn2c1] | ¹H NMR (400 MHz, CD₃OD) δ 9.02 (d, J = 2.7 Hz, 1H), 8.73 (s, 1H), 8.33 (d, J = 3.5 Hz, 1H), 8.14 (dt, J = 6.7, 1.5 Hz, 1H), 7.45-7.33 (m, 2H), 6.34-6.20 (m, 2H), 5.02-4.93 (m, 1H), 3.47 (s, 3H), 3.38 (s, 3H), 1.28 (d, J = 6.6 Hz, 6H). LCMS m/z = 486.2 (M + 1); RT = 2.98, Method 1. |
| 552 | 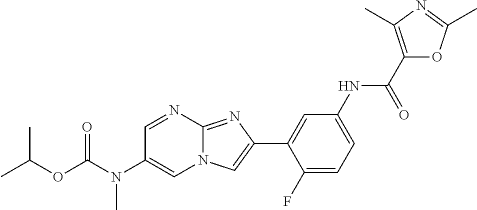 [SMILES: Cc1nc(C)c(C(=O)Nc2ccc(F)c(-c3cn4cc(N(C)C(=O)OC(C)C)cnc4n3)c2)o1] | ¹H NMR (400 MHz, CD₃OD) δ 9.16 (d, J = 2.7 Hz, 1H), 8.94 (s, 1H), 8.37 (dd, J = 8.1, 2.7 Hz, 2H), 7.78 (ddd, J = 8.9, 4.4, 2.7 Hz, 1H), 7.35 (dd, J = 10.7, 9.0 Hz, 1H), 5.07-4.96 (m, 1H), 3.42 (s, 3H), 2.56 (s, 3H), 2.48 (s, 3H), 1.31 (d, J = 6.3 Hz, 6H). LCMS m/z = 467.4 (M + 1); RT = 1.61, Method 3. |
| 553 | 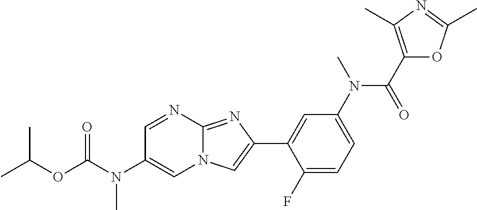 [SMILES: Cc1nc(C)c(C(=O)N(C)c2ccc(F)c(-c3cn4cc(N(C)C(=O)OC(C)C)cnc4n3)c2)o1] | LCMS m/z= 481.1 (M + 1); RT = 0.89, Method 2. |
| 554 | 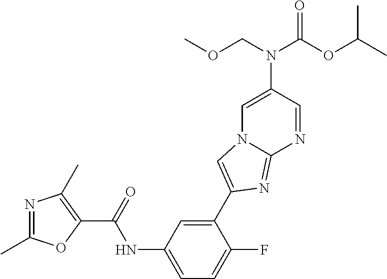 [SMILES: COCN(C(=O)OC(C)C)c1cnc2nc(-c3cc(NC(=O)c4oc(C)nc4C)ccc3F)cn2c1] | LCMS m/z = 497.4 (M + 1); RT = 1.48, Method 3. |
| 555 | 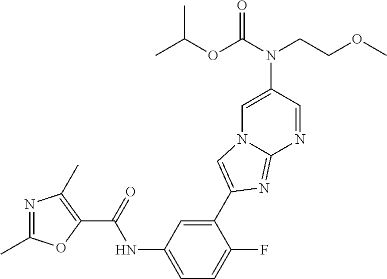 | ¹H NMR (400 MHz, CD₃OD) δ 9.07 (s, 1H), 8.71 (s, 1H), 8.33 (dd, J = 7.3, 3.0 Hz, 2H), 7.82 (ddd, J = 8.9, 4.4, 2.8 Hz, 1H), 7.31 (dd, J = 10.8, 9.0 Hz, 1H), 5.05-4.96 (m, 1H), 3.91 (t, J = 5.0 Hz, 2H), 3.64 (t, J = 5.1 Hz, 2H), 3.38 (s, 3H), 2.57 (s, 3H), 2.48 (s, 3H), 1.41-1.14 (m, 6H). LCMS m/z = 511.5 (M + 1); RT = 1.66, Method 3. |
| 556 | 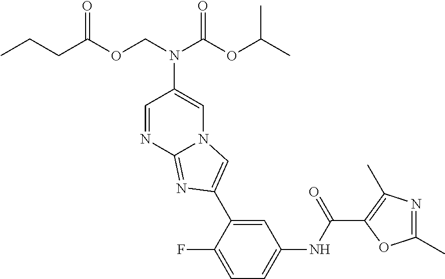 [SMILES: CCCC(=O)OCN(C(=O)OC(C)C)c1cnc2nc(-c3cc(NC(=O)c4oc(C)nc4C)ccc3F)cn2c1] | ¹H NMR (400 MHz, CD₃OD) δ 9.00 (d, J = 2.7 Hz, 1H), 8.55 (d, J = 2.5 Hz, 1H), 8.31-8.28 (m, 2H), 7.85 (ddd, J = 8.9, 4.4, 2.7 Hz, 1H), 7.26 (dd, J = 10.9, 9.0 Hz, 1H), 5.70 (s, 2H), 5.02 (hept, J = 5.9 Hz, 1H), 2.56 (s, 3H), 2.47 (s, 3H), 2.40 (t, J = 7.3 Hz, 2H), 1.70-1.59 (m, 2H), 1.36-1.21 (m, 6H), 0.96 (t, J = 7.4 Hz, 3H). LCMS m/z = 553.3 (M + 1); RT = 1.81, Method 3. |
| 557 | 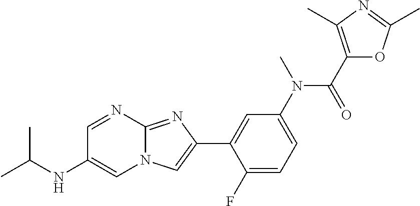 [SMILES: Cc1nc(C)c(C(=O)N(C)c2ccc(F)c(-c3cn4cc(NC(C)C)cnc4n3)c2)o1] | ¹H NMR (400 MHz, CD₃OD) δ 8.56 (d, J = 2.8 Hz, 1H), 8.26 (d, J = 2.7 Hz, 1H), 8.15 (d, J = 2.8 Hz, 1H), 7.90-7.80 (m, 1H), 7.47-7.39 (m, 2H), 3.56 (hept, J = 6.3 Hz, 1H), 3.48 (s, 3H), 2.32 (s, 3H), 2.12 (s, 3H), 1.29 (d, J = 6.3 Hz, 6H). LCMS m/z = 423.3 (M + 1); RT = 2.68, Method 1. |
| 558 | 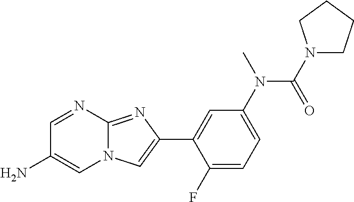 [SMILES: CN(C(=O)N1CCCC1)c1ccc(F)c(-c2cn3cc(N)cnc3n2)c1] | LCMS m/z = 355.3 (M + 1); RT = 1.15, Method 3. |
| 559 | 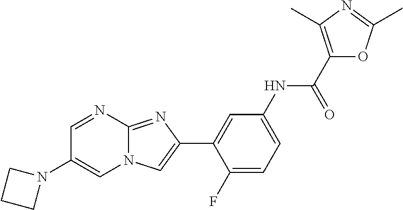 [SMILES: Cc1nc(C)c(C(=O)Nc2ccc(F)c(-c3cn4cc(N5CCC5)cnc4n3)c2)o1] | LCMS m/z = 407.3 (M + 1); RT = 2.74, Method 1. |
| 560 | 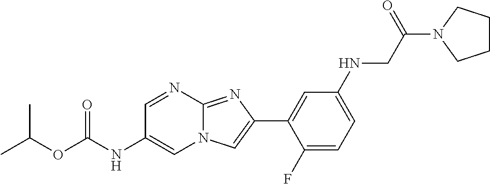 [SMILES: CC(C)OC(=O)Nc1cnc2nc(-c3cc(NCC(=O)N4CCCC4)ccc3F)cn2c1] | LCMS m/z = 441 (M + 1); RT = 0.87, Method 2. |
| 561 | 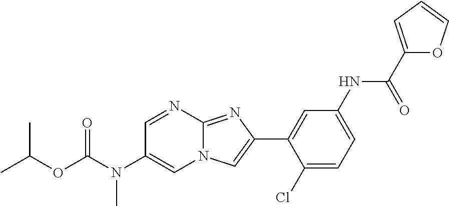 [SMILES: CC(C)OC(=O)N(C)c1cnc2nc(-c3cc(NC(=O)c4ccco4)ccc3Cl)cn2c1] | ¹H NMR (400 MHz, CD₃OD) δ 9.18 (d, J = 2.8 Hz, 1H), 8.96 (s, 1H), 8.48 (s, 1H), 8.32 (d, J = 2.6 Hz, 1H), 7.84 (dd, J = 8.8, 2.5 Hz, 1H), 7.78 (d, J = 1.8 Hz, 1H), 7.61 (d, J = 8.8 Hz, 1H), 7.32 (d, J = 3.5 Hz, 1H), 6.67 (dd, J = 3.5, 1.8 Hz, 1H), 5.01 (hept, J = 6.3 Hz, 1H), 3.43 (s, 3H), 1.31 (d, J = 6.3 Hz, 6H). LCMS m/z = 454 (M + 1); RT = 0.97, Method 2. |
| 562 | 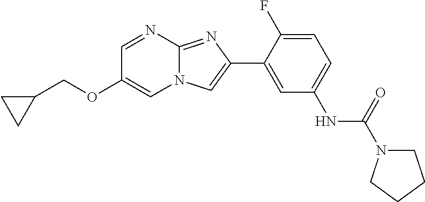 [SMILES: O=C(Nc1ccc(F)c(-c2cn3cc(OCC4CC4)cnc3n2)c1)N1CCCC1] | ¹H NMR (400 MHz, CD₃OD) δ 8.50 (d, J = 2.9 Hz, 1H), 8.44 (d, J = 2.7 Hz, 1H), 8.10 (d, J = 3.8 Hz, 1H), 7.94 (dd, J = 6.6, 2.7 Hz, 1H), 7.56 (ddd, J = 8.5, 4.3, 2.7 Hz, 1H), 7.14 (dd, J = 11.0, 8.9 Hz, 1H), 3.90 (d, J = 7.0 Hz, 2H), 3.54- 3.45 (m, 4H), 1.99 (q, J = 4.6, 2.9 Hz, 4H), 1.34 (td, J = 5.4, 2.5 Hz, 1H), 0.73-0.62 (m, 2H), 0.46-0.37 (m, 2H). LCMS m/z = 396.3 (M + 1); RT = 2.89, Method 1. |
| 563 | 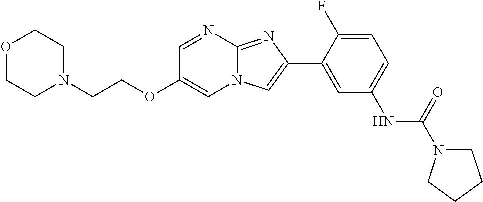 [SMILES: O=C(Nc1ccc(F)c(-c2cn3cc(OCCN4CCOCC4)cnc3n2)c1)N1CCCC1] | ¹H NMR (400 MHz, CD₃OD) δ 8.59 (d, J = 2.9 Hz, 1H), 8.46 (d, J = 2.8 Hz, 1H), 8.13 (d, J = 3.8 Hz, 1H), 7.94 (td, J = 6.5, 2.2 Hz, 1H), 7.57 (ddd, J = 8.8, 4.4, 2.8 Hz, 1H), 7.14 (dd, J = 10.9, 8.9 Hz, 1H), 4.22 (t, J = 5.3 Hz, 2H), 3.79- 3.67 (m, 4H), 3.57-3.42 (m, 4H), 2.88 (t, J = 5.3 Hz, 2H), 2.62 (t, J = 4.6 Hz, 4H), 2.05-1.91 (m, 4H). LCMS m/z = 455.3 (M + 1); RT = 1.11, Method 3. |
| 564 | 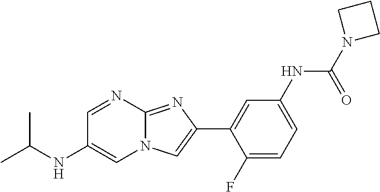 [SMILES: CC(C)Nc1cnc2nc(-c3cc(NC(=O)N4CCC4)ccc3F)cn2c1] | LCMS m/z = 369.1 (M + 1); RT = 0.71, Method 2. |
| 565 | 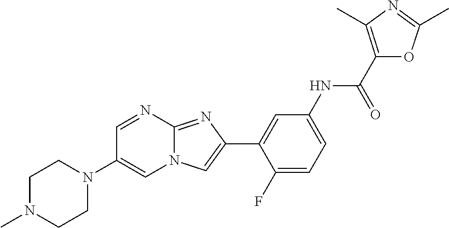 | LCMS m/z = 450.4 (M + 1); RT = 1.22, Method 3. |
| 566 | 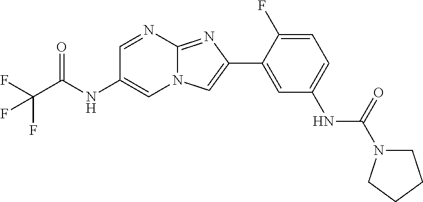 [SMILES: O=C(Nc1ccc(F)c(-c2cn3cc(NC(=O)C(F)(F)F)cnc3n2)c1)N1CCCC1] | ¹H NMR (400 MHz, DMSO-d₆) δ 11.80 (brs, 1H), 9.55 (d, J = 2.57 Hz, 1H), 8.67 (d, J = 2.69 Hz, 1H), 8.41 (d, J = 4.16 Hz, 1H), 8.37 (dd, J = 6.85, 2.81 Hz, 1H), 8.34 (s, 1H), 7.65 (ddd, J = 8.89, 4.49, 2.87 Hz, 1H), 7.19 (dd, J = 11.06, 8.99 Hz, 1H), 3.39 (t, J = 6.60 Hz, 4H), 1.83-1.89 (m, 4H). LCMS m/z = 437.3 (M + 1); RT = 1.46, Method 3. |
| 567 | 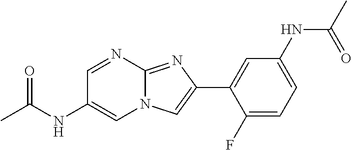 [SMILES: CC(=O)Nc1ccc(F)c(-c2cn3cc(NC(C)=O)cnc3n2)c1] | ¹H NMR (400 MHz, DMSO-d₆) δ 10.38 (s, 1H), 10.10 (s, 1H), 9.54 (d, J = 2.57 Hz, 1H), 8.49 (d, J = 2.57 Hz, 1H), 8.42 (dd, J = 6.79, 2.63 Hz, 1H), 8.38 (d, J = 4.28 Hz, 1H), 7.64-7.70 (m, 1H), 7.25 (dd, J = 10.94, 8.99 Hz, 1H), 2.12 (s, 3H), 2.06 (s, 3H). LCMS m/z = 328.3 (M + 1); RT = 1.09, Method 3. |
| 568 | 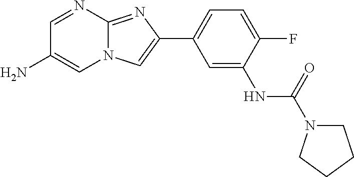 [SMILES: Nc1cnc2nc(-c3ccc(F)c(NC(=O)N4CCCC4)c3)cn2c1] | ¹H NMR (400 MHz, DMSO-d₆) δ 8.18 (d, J = 2.81 Hz, 1H), 8.09-8.13 (m, 2H), 8.04 (d, J = 2.81 Hz, 1H), 7.87 (s, 1H), 7.61 (ddd, J = 8.47, 4.74, 2.20 Hz, 1H), 7.23 (dd, J = 10.51, 8.44 Hz, 1H), 5.17 (s, 2H), 3.40 (s, 4H), 1.88 (t, J = 6.54 Hz, 4H). |
| 569 | 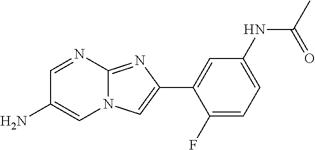 [SMILES: CC(=O)Nc1ccc(F)c(-c2cn3cc(N)cnc3n2)c1] | ¹H NMR (400 MHz, DMSO-d₆) δ 10.15 (brs, 1H), 8.38 (dd, J = 6.85, 2.69 Hz, 1H), 8.24 (d, J = 2.57 Hz, 1H), 8.09 (dd, J = 8.31, 3.06 Hz, 2H), 7.64 (ddd, J = 8.80, 4.46, 2.87 Hz, 1H), 7.21 (dd, J = 11.07, 8.86 Hz, 1H), 5.24 (s, 2H), 2.06 (s, 3H). LCMS m/z = 286.3 (M + 1); RT = 0.94, Method 3. |
| 570 | 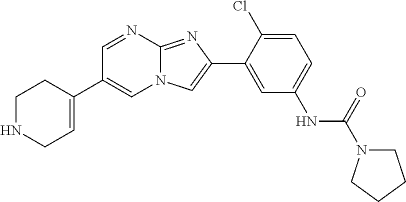 [SMILES: O=C(Nc1ccc(Cl)c(-c2cn3cc(C4=CCNCC4)cnc3n2)c1)N1CCCC1] | ¹H NMR (400 MHz, DMSO-d₆) δ 8.98 (d, J = 2.5 Hz, 1H), 8.86 (d, J = 2.5 Hz, 1H), 8.51 (s, 1H), 8.48-8.39 (m, 2H), 7.72 (dt, J = 8.8, 2.3 Hz, 1H), 7.39 (d, J = 8.8 Hz, 1H), 6.50 (s, 1H), 4.05 (s, 1H), 3.39 (q, J = 6.3, 4.5 Hz, 4H), 3.32 (s, 2H), 2.94 (t, J = 5.6 Hz, 2H), 2.39- 2.27 (m, 2H), 1.93-1.78 (m, 4H). LCMS m/z = 423 (M + 1); RT = 0.57, Method 2. |
| 571 | 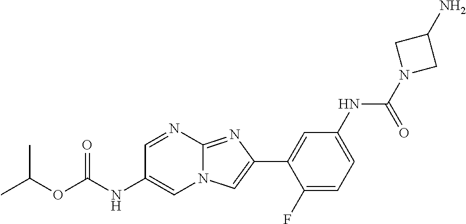 | LCMS m/z = 542.1 (M + 1); RT = 0.99, Method 2. |
| 572 | 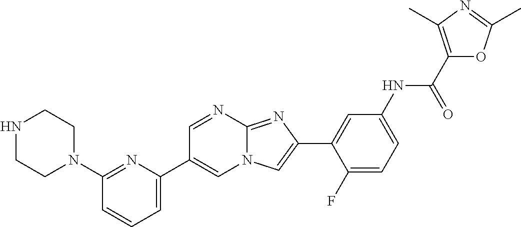 [SMILES: Cc1nc(C)c(C(=O)Nc2ccc(F)c(-c3cn4cc(-c5cccc(N6CCNCC6)n5)cnc4n3)c2)o1] | LCMS m/z = 513.1 (M + 1); RT = 0.73, Method 2. |
| 573 | 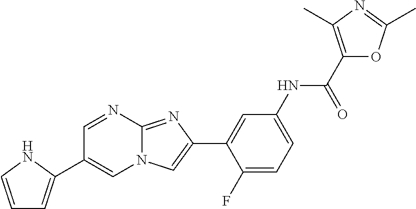 [SMILES: Cc1nc(C)c(C(=O)Nc2ccc(F)c(-c3cn4cc(-c5ccc[nH]5)cnc4n3)c2)o1] | ¹H NMR (400 MHz, CD₃OD) δ 9.12 (d, J = 2.4 Hz, 1H), 9.07 (d, J = 2.4 Hz, 1H), 8.41-8.28 (m, 2H), 7.78 (ddd, J = 8.9, 4.4, 2.7 Hz, 1H), 7.34 (dd, J = 10.7, 9.0 Hz, 1H), 7.00 (dd, J = 2.6, 1.3 Hz, 1H), 6.75 (dd, J = 3.5, 1.2 Hz, 1H), 6.42-6.16 (m, 1H), 2.57 (s, 3H), 2.48 (s, 3H). LCMS m/z = 417 (M + 1); RT = 0.94, Method 2. |
| 574 | 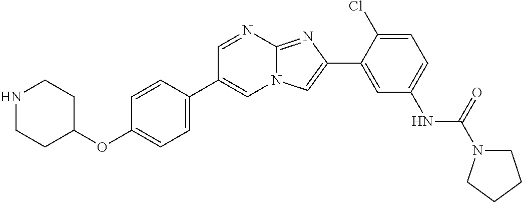 [SMILES: O=C(Nc1ccc(Cl)c(-c2cn3cc(-c4ccc(OC5CCNCC5)cc4)cnc3n2)c1)N1CCCC1] | ¹H NMR (400 MHz, CD₃OD) δ 9.03 (d, J = 2.4 Hz, 1H), 8.85 (d, J = 2.4 Hz, 1H), 8.40 (s, 1H), 8.00 (d, J = 2.6 Hz, 1H), 7.67-7.61 (m, 2H), 7.59 (dd, J = 8.8, 2.7 Hz, 1H), 7.40 (d, J = 8.8 Hz, 1H), 7.15- 7.06 (m, 2H), 4.56 (tt, J = 7.9, 3.8 Hz, 1H), 3.54-3.43 (m, 4H), 3.11 (ddd, J = 12.6, 6.1, 4.0 Hz, 2H), 2.77 (ddd, J = 12.6, 9.0, 3.3 Hz, 2H), 2.10-2.01 (m, 2H), 2.01-1.93 (m, 4H), 1.71 (dtd, J = 12.9, 8.6, 3.7 Hz, 2H). LCMS m/z = 517.1 (M + 1); RT = 0.71, Method 2. |
| 575 | 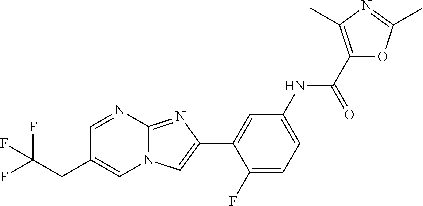 [SMILES: Cc1nc(C)c(C(=O)Nc2ccc(F)c(-c3cn4cc(CC(F)(F)F)cnc4n3)c2)o1] | ¹H NMR (400 MHz, DMSO-d₆) δ 10.34 (s, 1H), 9.08 (d, J = 2.3 Hz, 1H), 8.73 (dd, J = 6.8, 2.8 Hz, 1H), 8.58 (d, J = 2.3 Hz, 1H), 8.38 (d, J = 4.3 Hz, 1H), 7.80 (ddd, J = 9.0, 4.5, 2.8 Hz, 1H), 7.33 (dd, J = 10.9, 9.0 Hz, 1H), 3.86 (q, J = 11.3 Hz, 2H), 2.51 (s, 3H), 2.40 (s, 3H). LCMS m/z = 434.3 (M + 1); RT = 2.92, Method 1. |
| 576 | 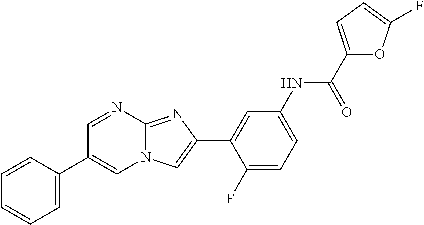 [SMILES: O=C(Nc1ccc(F)c(-c2cn3cc(-c4ccccc4)cnc3n2)c1)c1ccc(F)o1] | LCMS m/z = 417 (M + 1); RT = 1.04, Method 2. |
| 577 | 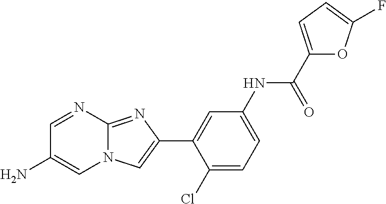 [SMILES: Nc1cnc2nc(-c3cc(NC(=O)c4ccc(F)o4)ccc3Cl)cn2c1] | LCMS m/z = 371.9 (M + 1); RT = 0.67, Method 2. |
| 578 | 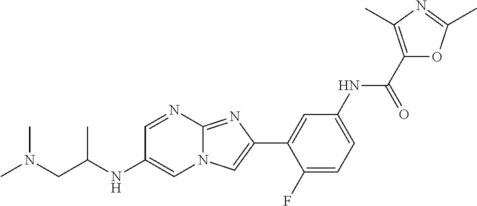 | MS m/z = 452.2 (M + 1). |
| 579 | 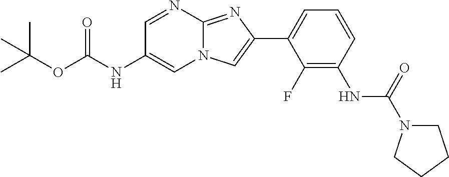 [SMILES: CC(C)(C)OC(=O)Nc1cnc2nc(-c3cccc(NC(=O)N4CCCC4)c3F)cn2c1] | ¹H NMR (400 MHz, DMSO-d₆) δ 9.78 (brs, 1H), 9.34 (brs, 1H), 8.46 (d, J = 2.69 Hz, 1H), 8.32 (d, J = 4.40 Hz, 1H) 7.90-7.97 (m, 2H), 7.51 (td, J = 7.73, 1.65 Hz, 1H), 7.21 (t, J = 7.89 Hz, 1H), 3.40 (t, J = 6.60 Hz, 4H), 1.88 (t, J = 6.66 Hz, 4H), 1.51 (s, 9H). LCMS m/z = 441.4 (M + 1); RT = 1.46, Method 3. |
| 580 | 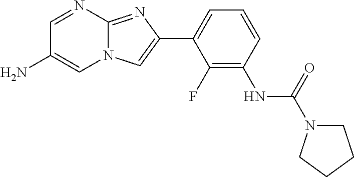 [SMILES: Nc1cnc2nc(-c3cccc(NC(=O)N4CCCC4)c3F)cn2c1] | ¹H NMR (400 MHz, DMSO-d₆) δ 9.78 (brs, 1H), 9.34 (brs, 1H), 8.46 (d, J = 2.69 Hz, 1H), 8.32 (d, J = 4.40 Hz, 1H), 7.90-7.97 (m, 2H), 7.51 (td, J = 7.73, 1.65 Hz, 1H), 7.21 (t, J = 7.89 Hz, 1H), 3.40 (t, J = 6.60 Hz, 4H), 1.88 (t, J = 6.66 Hz, 4H), 1.51 (s, 9H). LCMS m/z = 341.3 (M + 1); RT = 1.02, Method 3. |
| 581 | 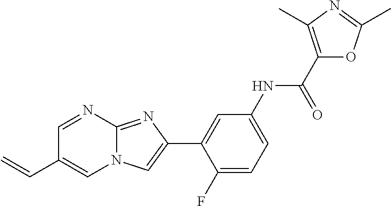 [SMILES: C=Cc1cnc2nc(-c3cc(NC(=O)c4oc(C)nc4C)ccc3F)cn2c1] | ¹H NMR (400 MHz, DMSO-d₆) δ 10.32 (s, 1H), 9.07 (d, J = 2.26 Hz, 1H), 8.90 (d, J = 2.26 Hz, 1H), 8.73 (dd, J = 6.78, 2.51 Hz, 1H), 8.26 (d, J = 4.27 Hz, 1H), 7.80 (dd, J = 7.53, 4.27 Hz, 1H), 7.33 (dd, J = 11.04, 9.03 Hz, 1H), 6.78 (dd, J = 17.69, 11.17 Hz, 1H), 6.05 (d, J = 17.82 Hz, 1H), 5.45 (d, J = 11.29 Hz, 1H), 2.48 (s, 3H), 2.41 (s, 3H). LCMS m/z = 378.3 (M + 1); RT = 1.54, Method 3. |
| 582 | 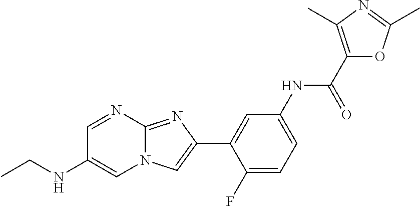 | ¹H NMR (400 MHz, DMSO-d₆) δ 10.19- 10.38 (m, 1H), 8.52-8.76 (m, 1H), 8.19- 8.41 (m, 1H), 7.92-8.16 (m, 2H), 7.63- 7.83 (m, 1H), 7.17-7.38 (m, 1H), 5.85 (brs, 1H), 2.87-3.09 (m, 2H), 2.46-2.50 (m, 3H), 2.40 (s, 3H), 1.23 (s, 3H). LCMS m/z = 378.3 (M + 1); RT = 1.54, Method 3. |
| 583 | 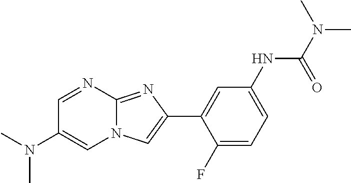 | ¹H NMR (400 MHz, DMSO-d₆) δ 8.60 (brs, 1H), 8.46 (brs, 1H), 8.30 (brs, 2H), 8.09 (brs, 1H), 7.54 (m, 1H), 7.17 (t, J = 9.54 Hz, 1H), 2.95 (brs, 6H), 2.88 (brs, 6H). LCMS m/z = 343.1 (M + 1); RT = 0.62, Method 4. |
| 584 | 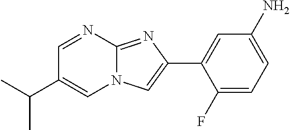 [SMILES: CC(C)c1cnc2nc(-c3cc(N)ccc3F)cn2c1] | ¹H NMR (400 MHz, DMSO-d₆) δ 8.84 (d, J = 2.5 Hz, 1H), 8.56 (d, J = 2.5 Hz, 1H), 8.11 (d, J = 4.1 Hz, 1H), 7.50 (dd, J = 6.5, 3.1 Hz, 1H), 6.96 (dd, J = 11.4, 8.7 Hz, 1H), 6.53 (dq, J = 8.8, 3.0 Hz, 1H), 3.01 (p, J = 6.9 Hz, 1H), 1.29 (d, J = 6.8 Hz, 6H). ¹⁹F NMR (376 MHz, DMSO-d₆) δ −130.47. LCMS m/z = 271.3 (M + 1); RT = 1.15, Method 3. |
| 585 | 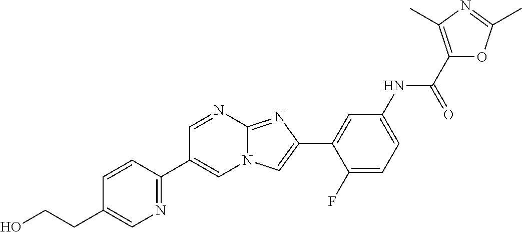 [SMILES: Cc1nc(C)c(C(=O)Nc2ccc(F)c(-c3cn4cc(-c5ccc(CCO)cn5)cnc4n3)c2)o1] | ¹H NMR (400 MHz, DMSO-d₆) δ 10.36 (s, 1H), 9.69 (d, J = 2.4 Hz, 1H), 9.28 (d, J = 2.4 Hz, 1H), 8.76 (dd, J = 7.0, 2.7 Hz, 1H), 8.59 (d, J = 2.3 Hz, 1H), 8.40 (d, J = 4.1 Hz, 1H), 8.00 (d, J = 8.1 Hz, 1H), 7.89-7.76 (m, 2H), 7.34 (dd, J = 11.0, 9.0 Hz, 1H), 4.76 (t, J = 5.1 Hz, 1H), 3.67 (td, J = 6.6, 5.1 Hz, 2H), 2.81 (t, J = 6.6 Hz, 2H), 2.51 (s, 3H), 2.41 (s, 3H). LCMS m/z = 473.3 (M + 1); RT = 1.44, Method 3. |
| 586 | 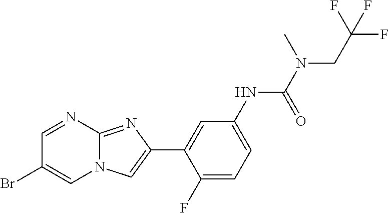 [SMILES: CN(CC(F)(F)F)C(=O)Nc1ccc(F)c(-c2cn3cc(Br)cnc3n2)c1] | ¹H NMR (400 MHz, DMSO-d₆) δ 9.36 (s, 1H), 8.64 (s, 1H), 8.50 (s, 1H), 8.40 (dd, J = 6.78, 2.76 Hz, 1H), 8.23 (d, J = 4.02 Hz, 1H), 7.61-7.74 (m, 1H), 7.24 (dd, J = 11.04, 9.03 Hz, 1H), 5.27-5.50 (m, 1H), 3.39-3.83 (m, 4H), 1.98-2.28 (m, 2H). LCMS m/z = 446.2 (M + 1), 448.2 (M + 3), RT = 1.63, Method 3. |
| 587 | 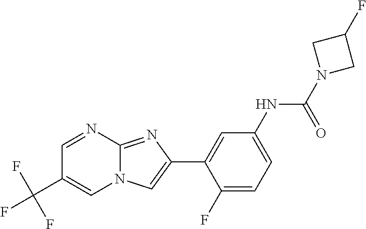 [SMILES: O=C(Nc1ccc(F)c(-c2cn3cc(C(F)(F)F)cnc3n2)c1)N1CC(F)C1] | ¹H NMR (400 MHz, DMSO-d₆) δ 9.65 (s, 1H), 8.89 (d, J = 2.26 Hz, 1H), 8.85 (s, 1H), 8.34-8.43 (m, 2H), 7.63-7.74 (m, 1H), 7.26 (dd, J = 10.79, 9.03 Hz, 1H), 5.26-5.52 (m, 1H), 4.21-4.41 (m, 2H), 3.91-4.11 (m, 2H). LCMS m/z = 398.3 (M + 1); RT = 0.88, Method 2. |
| 588 | 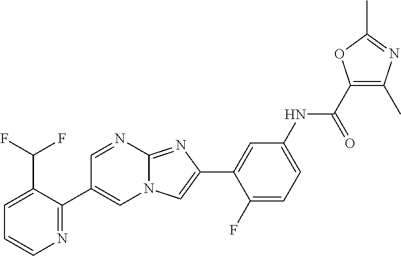 [SMILES: Cc1nc(C)c(C(=O)Nc2ccc(F)c(-c3cn4cc(-c5ncccc5C(F)F)cnc4n3)c2)o1] | ¹H NMR (400 MHz, CD₃OD) δ 9.11 (d, J = 2.5 Hz, 1H), 8.92-8.85 (m, 1H), 8.82 (d, J = 2.4 Hz, 1H), 8.39 (d, J = 3.8 Hz, 1H), 8.35 (dd, J = 6.6, 2.8 Hz, 1H), 8.32-8.26 (m, 1H), 7.87 (ddd, J = 8.9, 4.5, 2.8 Hz, 1H), 7.69 (dd, J = 8.0, 4.8 Hz, 1H), 7.29 (dd, J = 10.9, 8.9 Hz, 1H), 7.02 (t, J = 53.9 Hz, 1H), 2.57 (s, 3H), 2.48 (s, 3H). LCMS m/z = 479.3 (M + 1); RT = 1.59, Method 3. |
| 589 | 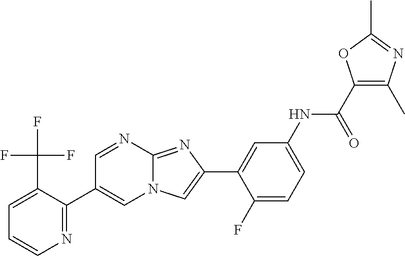 [SMILES: Cc1nc(C)c(C(=O)Nc2ccc(F)c(-c3cn4cc(-c5ncccc5C(F)(F)F)cnc4n3)c2)o1] | 1H NMR (400 MHz, CD₃OD) δ 9.15 (d, J = 2.4 Hz, 1H), 8.97 (dd, J = 5.0, 1.5 Hz, 1H), 8.75 (d, J = 2.4 Hz, 1H), 8.45- 8.28 (m, 3H), 7.87 (ddd, J = 8.9, 4.4, 2.7 Hz, 1H), 7.75 (ddd, J = 8.2, 4.9, 1.0 Hz, 1H), 7.28 (dd, J = 10.9, 8.9 Hz, 1H), 2.57 (s, 3H), 2.48 (s, 3H). LCMS m/z = 497.3 (M + 1); RT = 1.65, Method 3. |
| 590 | 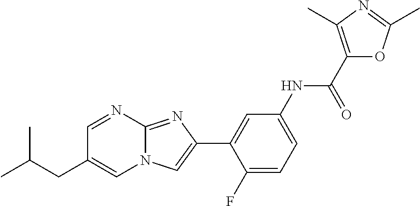 [SMILES: Cc1nc(C)c(C(=O)Nc2ccc(F)c(-c3cn4cc(CC(C)C)cnc4n3)c2)o1] | ¹H NMR (400 MHz, CD₃OD) δ 8.70 (d, J = 2.3 Hz, 1H), 8.49 (d, J = 2.4 Hz, 1H), 8.25 (dd, J = 6.6, 2.7 Hz, 1H), 8.18 (d, J = 3.9 Hz, 1H), 7.84 (ddd, J = 8.9, 4.5, 2.8 Hz, 1H), 7.24 (dd, J = 10.9, 8.9 Hz, 1H), 2.59 (d, J = 7.2 Hz, 2H), 2.56 (s, 3H), 2.47 (s, 3H), 1.97 (dd, J = 13.6, 6.9 Hz, 1H), 1.00 (d, J = 6.6 Hz, 6H). LCMS m/z = 408.1 (M + 1); RT = 0.98, Method 2. |
| 591 | 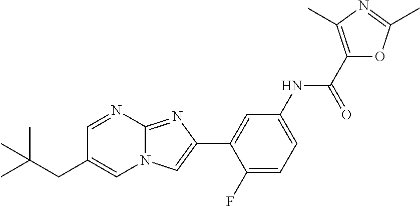 [SMILES: Cc1nc(C)c(C(=O)Nc2ccc(F)c(-c3cn4cc(CC(C)(C)C)cnc4n3)c2)o1] | ¹H NMR (400 MHz, CD₃OD) δ 8.70 (d, J = 2.3 Hz, 1H), 8.46 (d, J = 2.4 Hz, 1H), 8.25 (dd, J = 6.6, 2.7 Hz, 1H), 8.19 (d, J = 3.9 Hz, 1H), 7.83 (dq, J = 7.2, 2.7, 2.2 Hz, 1H), 7.24 (dd, J = 11.0, 9.0 Hz, 1H), 2.61 (s, 2H), 2.56 (s, 3H), 2.47 (s, 3H), 1.00 (s, 9H). LCMS m/z = 422.4 (M + 1); RT = 1.02, Method 2. |
| 592 | 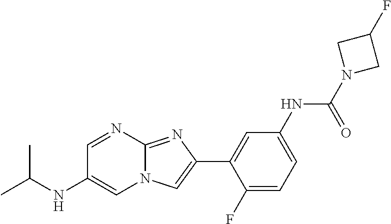 [SMILES: CC(C)Nc1cnc2nc(-c3cc(NC(=O)N4CC(F)C4)ccc3F)cn2c1] | ¹H NMR (400 MHz, DMSO-d₆) δ 8.79 (s, 1H), 8.24-8.28 (m, 2H), 8.03-8.07 (m, 2H), 7.6 (t, J = 8.0 Hz, 1H), 7.18 (t, J = 8.0 Hz, 1H), 5.68 (d, J = 4.0 Hz, 1H), 5.31-5.48 (m, 1H), 4.28-4.34 (m, 2H), 3.97-4.06 (m, 2H), 1.18 (d, J = 4.0 Hz, 6H). LCMS m/z = 387.4 (M + 1); RT = 1.35, Method 3. |
| 593 | 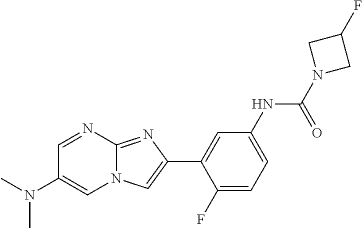 [SMILES: CN(C)c1cnc2nc(-c3cc(NC(=O)N4CC(F)C4)ccc3F)cn2c1] | ¹H NMR (400 MHz, DMSO-d₆) δ 8.79 (s, 1H), 8.62 (t, J = 8.0 Hz, 1H), 8.31 (t, J = 8.0 Hz, 2H), 8.10 (d, J = 4.0 Hz, 1H), 7.60-7.64 (m, 1H), 7.17-7.22 (m, 1H), 5.31-5.48 (m, 1H), 4.26-4.33 (m, 2H), 3.98-4.06 (m, 2H), 2.89 (s, 6H). LCMS m/z = 373.3 (M + 1); RT = 1.28, Method 3. |
| 594 | 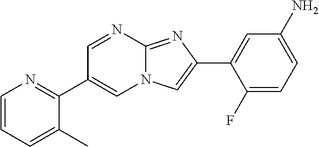 [SMILES: Cc1cccnc1-c1cnc2nc(-c3cc(N)ccc3F)cn2c1] | ¹H NMR (DMSO-d₆) δ 9.29 (d, J = 2.51 Hz, 1H), 8.82 (d, J = 2.51 Hz, 1H), 8.58 (d, J = 4.02 Hz, 1H), 8.27 (d, J = 4.02 Hz, 1H), 7.82 (d, J = 7.53 Hz, 1H), 7.54-7.57 (m, 1H), 7.39-7.42 (m, 1H), 6.98-7.03 (m, 1H), 6.55-6.58 (m, 1H), 5.15 (s, 2H) 2.49 (s, 3H). LCMS m/z = 320.3 (M + 1); RT = 0.6, Method 2. |
| 595 | 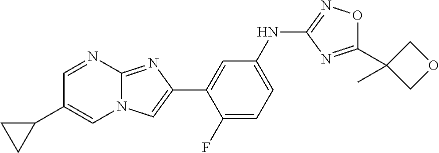 [SMILES: CC1(c2nc(Nc3ccc(F)c(-c4cn5cc(C6CC6)cnc5n4)c3)no2)COC1] | ¹H NMR (400 MHz, CD₃OD) δ 8.92- 8.78 (m, 2H), 8.32 (d, J = 2.6 Hz, 1H), 8.04 (dd, J = 6.3, 2.8 Hz, 1H), 7.60 (ddd, J = 9.0, 4.2, 2.7 Hz, 1H), 7.33 (dd, J = 10.6, 9.0 Hz, 1H), 5.07 (d, J = 6.0 Hz, 2H), 4.65 (d, J = 6.0 Hz, 2H), 2.28-2.05 (m, 1H), 1.84 (s, 3H), 1.30-1.12 (m, 2H), 1.03-0.85 (m, 2H). LCMS m/z = 407.4 (M + 1); RT = 0.94. Method 2. |
| 596 | 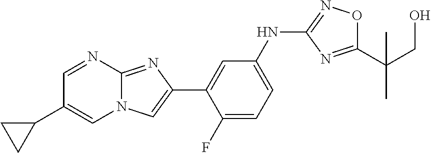 [SMILES: CC(C)(CO)c1nc(Nc2ccc(F)c(-c3cn4cc(C5CC5)cnc4n3)c2)no1] | ¹H NMR (400 MHz, CD₃OD) δ 8.94- 8.82 (m, 2H), 8.33 (d, J = 2.5 Hz, 1H), 8.07-7.96 (m, 1H), 7.62-7.52 (m, 1H), 7.33 (dd, J = 10.7, 9.1 Hz, 1H), 3.73 (s, 2H), 2.25-2.13 (m, 1H), 1.41 (s, 6H), 1.25-1.12 (m, 2H), 0.95 (dt, J = 6.6, 4.8 Hz, 2H). LCMS m/z = 409.4 (M + 1); RT = 0.92, Method 2. |
| 597 | 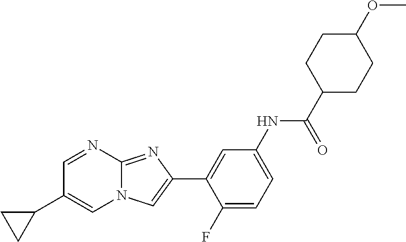 [SMILES: COC1CCC(C(=O)Nc2ccc(F)c(-c3cn4cc(C5CC5)cnc4n3)c2)CC1] | LCMS m/z = 409.4 (M + 1); RT = 1.44, Method 3. |
| 598 | 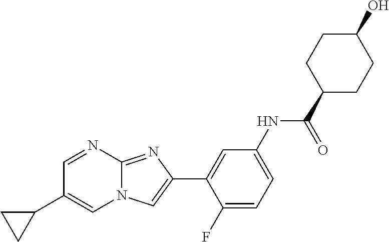 [SMILES: O=C(Nc1ccc(F)c(-c2cn3cc(C4CC4)cnc3n2)c1)[C@H]1CC[C@@H](O)CC1] | LCMS m/z = 395.1 (M + 1); RT = 1.29, Method 3. |
| 599 | 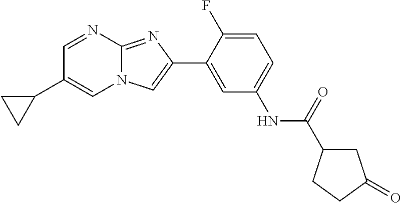 [SMILES: O=C1CCC(C(=O)Nc2ccc(F)c(-c3cn4cc(C5CC5)cnc4n3)c2)C1] | LCMS m/z = 379.4 (M + 1); RT = 0.81, Method 2. |
| 600 | 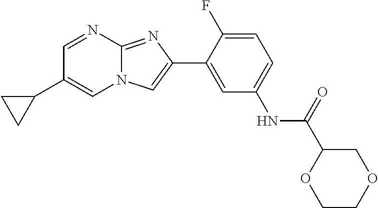 [SMILES: O=C(Nc1ccc(F)c(-c2cn3cc(C4CC4)cnc3n2)c1)C1COCCO1] | ¹H NMR (400 MHz, CD₃OD) δ 9.12- 8.61 (m, 2H), 8.43-8.12 (m, 2H), 7.70 (ddd, J = 9.0, 4.5, 2.7 Hz, 1H), 7.35 (dd, J = 10.7, 9.0 Hz, 1H), 4.30 (dd, J = 9.8, 3.1 Hz, 1H), 4.15-3.98 (m, 2H), 3.91-3.52 (m, 4H), 2.37-2.06 (m, 1H), 1.25-1.11 (m, 2H), 1.08-0.74 (m, 2H). LCMS m/z = 383.2 (M + 1); RT = 1.29, Method 3. |
| 601 | 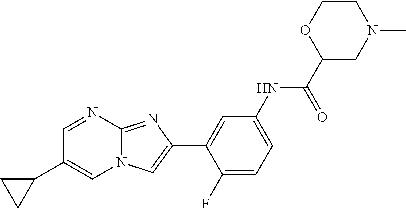 [SMILES: CN1CCOC(C(=O)Nc2ccc(F)c(-c3cn4cc(C5CC5)cnc4n3)c2)C1] | ¹H NMR (400 MHz, CD₃OD) δ 9.11- 8.58 (m, 2H), 8.47-8.08 (m, 2H), 7.73 (dq, J = 7.4, 2.3 Hz, 1H), 7.36 (dd, J = 10.7, 8.9 Hz, 1H), 4.45 (dd, J = 76.4, 10.8 Hz, 2H), 3.98 (q, J = 19.3, 15.8 Hz, 1H), 3.66-3.44 (m, 2H), 3.31 (dq, J = 3.2, 1.6 Hz, 2H), 3.02 (s, 3H), 2.16 (tt, J = 8.6, 5.1 Hz, 1H), 1.30-1.05 (m, 2H), 1.07-0.69 (m, 2H). LCMS m/z = 396.3 (M + 1); RT = 1.15, Method 3. |
| 602 | 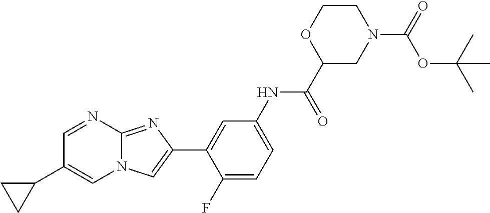 [SMILES: CC(C)(C)OC(=O)N1CCOC(C(=O)Nc2ccc(F)c(-c3cn4cc(C5CC5)cnc4n3)c2)C1] | LCMS m/z = 482.3 (M + 1); RT = 1.65, Method 3. |
| 603 | 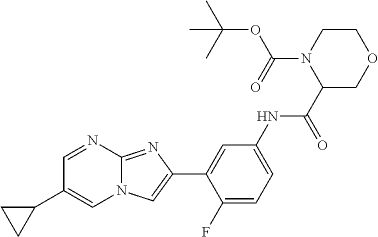 [SMILES: CC(C)(C)OC(=O)N1CCOCC1C(=O)Nc1ccc(F)c(-c2cn3cc(C4CC4)cnc3n2)c1] | LCMS m/z = 482.4 (M + 1); RT = 1.57, Method 3. |
| 604 | 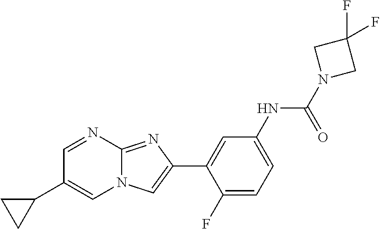 [SMILES: O=C(Nc1ccc(F)c(-c2cn3cc(C4CC4)cnc3n2)c1)N1CC(F)(F)C1] | LCMS m/z = 388.2 (M + 1); RT = 2.86, Method 1. |
| 605 | 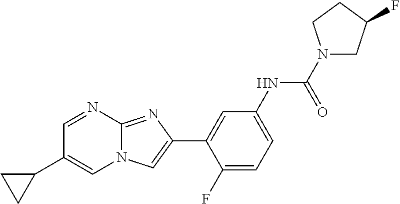 [SMILES: O=C(Nc1ccc(F)c(-c2cn3cc(C4CC4)cnc3n2)c1)N1CC[C@@H](F)C1] | LCMS m/z = 384.3 (M + 1); RT = 1.35, Method 3. |
| 606 | 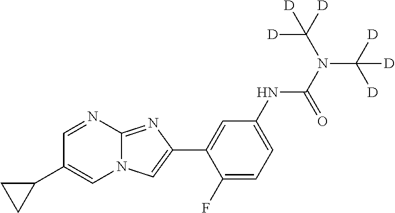 [SMILES: [2H]C([2H])([2H])N(C(=O)Nc1ccc(F)c(-c2cn3cc(C4CC4)cnc3n2)c1)C([2H])([2H])[2H]] | LCMS m/z= 346.4 (M + 1); RT = 1.29, Method 3. |
| 607 | 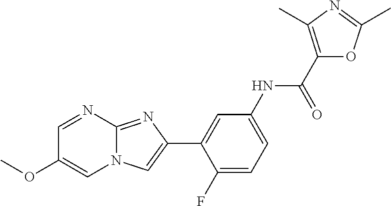 | ¹H NMR (400 MHz, DMSO-d₆) δ 10.35 (s, 1H), 8.80 (q, J = 2.2, 1.4 Hz, 1H), 8.68 (ddd, J = 7.0, 2.6, 1.4 Hz, 1H), 8.61-8.48 (m, 1H), 8.43-8.05 (m, 1H), 7.78 (ddd, J = 8.9, 4.5, 2.8 Hz, 1H), 7.34 (ddd, J = 10.7, 9.1, 1.3 Hz, 1H), 3.87 (s, 3H), 2.40 (s, 3H), 2.39 (s, 3H). LCMS m/z = 382.3 (M + 1); RT = 0.77, Method 2. |
| 608 | 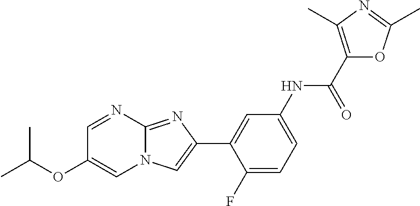 [SMILES: Cc1nc(C)c(C(=O)Nc2ccc(F)c(-c3cn4cc(OC(C)C)cnc4n3)c2)o1] | ¹H NMR (400 MHz, DMSO-d₆) δ 10.34 (s, 1H), 8.81 (d, J = 2.9 Hz, 1H), 8.68 (dd, J = 6.9, 2.7 Hz, 1H), 8.47 (d, J = 2.9 Hz, 1H), 8.20 (d, J = 4.2 Hz, 1H), 7.78 (ddd, J = 8.9, 4.5, 2.8 Hz, 1H), 7.33 (dd, J = 11.0, 8.9 Hz, 1H), 4.55 (p, J = 6.0 Hz, 1H), 2.40 (s, 3H), 2.39 (s, 3H), 1.35 (dd, J = 6.0, 2.8 Hz, 6H). LCMS m/z = 410.3 (M + 1); RT = 1.52, Method 3. |
| 609 | 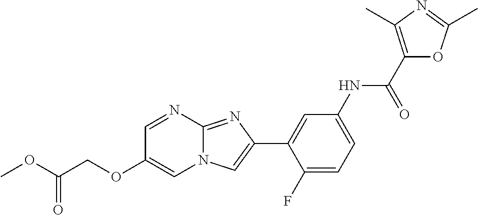 | ¹H NMR (400 MHz, DMSO-d₆) δ 10.34 (s, 1H), 8.83 (d, J = 3.0 Hz, 1H), 8.69 (dd, J = 6.9, 2.8 Hz, 1H), 8.60 (d, J = 2.9 Hz, 1H), 8.22 (d, J = 4.1 Hz, 1H), 7.79 (ddd, J = 8.9, 4.5, 2.8 Hz, 1H), 7.33 (dd, J = 11.0, 8.9 Hz, 1H), 4.92 (s, 2H), 3.75 (s, 3H), 2.51 (s, 3H), 2.40 (s, 3H). LCMS m/z = 440 (M + 1); RT = 0.8, Method 2. |
| 610 | 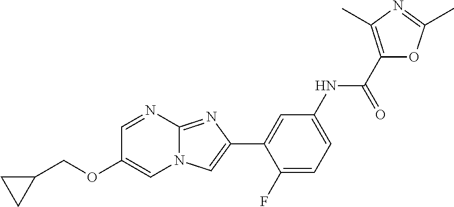 [SMILES: Cc1nc(C)c(C(=O)Nc2ccc(F)c(-c3cn4cc(OCC5CC5)cnc4n3)c2)o1] | LCMS m/z = 422.3 (M + 1); RT = 1.57, Method 3. |
| 611 | 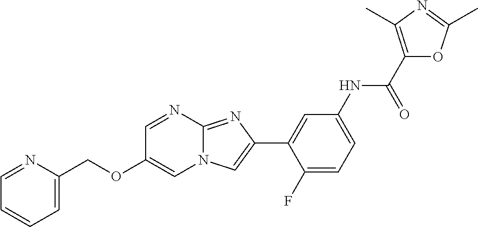 [SMILES: Cc1nc(C)c(C(=O)Nc2ccc(F)c(-c3cn4cc(OCc5ccccn5)cnc4n3)c2)o1] | ¹H NMR (400 MHz, DMSO-d₆) δ 10.35 (s, 1H), 8.91 (d, J = 3.0 Hz, 1H), 8.80- 8.52 (m, 3H), 8.26 (d, J = 4.1 Hz, 1H), 7.95 (td, J = 8.0, 1.5 Hz, 1H), 7.78 (ddd, J = 8.9, 4.5, 2.8 Hz, 1H), 7.67 (d, J = 7.8 Hz, 1H), 7.45 (dd, J = 7.5, 4.9 Hz, 1H), 7.34 (dd, J = 11.0, 9.0 Hz, 1H), 5.28 (s, 2H), 2.51 (s, 3H), 2.41 (s, 3H). LCMS m/z = 459.4 (M + 1); RT = 1.37, Method 3. |
| 612 | 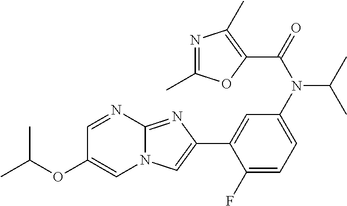 [SMILES: Cc1nc(C)c(C(=O)N(c2ccc(F)c(-c3cn4cc(OC(C)C)cnc4n3)c2)C(C)C)o1] | LCMS m/z = 452.1 (M + 1); RT = 1.00, Method 2b. |
| 613 | 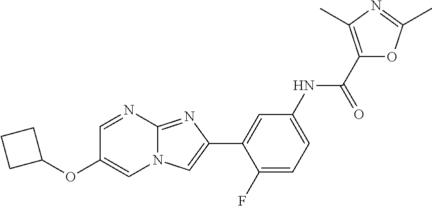 [SMILES: Cc1nc(C)c(C(=O)Nc2ccc(F)c(-c3cn4cc(OC5CCC5)cnc4n3)c2)o1] | ¹H NMR (400 MHz, CD₃OD): 8.89 (dd, J = 2.2, 1.0 Hz, 1H), 8.77 (d, J = 2.3 Hz, 1H), 8.42- 8.30 (m, 2H), 7.77 (ddd, J = 9.0, 4.5, 2.7 Hz, 1H), 7.36 (dd, J = 10.7, 9.0 Hz, 1H), 2.90 (d, J = 7.6 Hz, 2H), 2.80-2.63 (m, 1H), 2.56 (s, 3H), 2.48 (s, 3H), 2.21-2.08 (m, 2H), 2.01- 1.77 (m, 4H). |
| 614 | 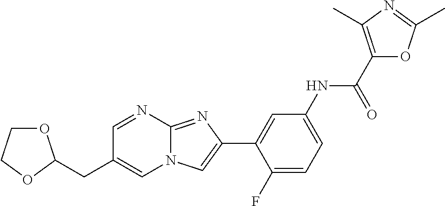 [SMILES: Cc1nc(C)c(C(=O)Nc2ccc(F)c(-c3cn4cc(CC5OCCO5)cnc4n3)c2)o1] | LCMS m/z = 438 (M + 1); RT = 0.79, Method 2. |
| 615 | 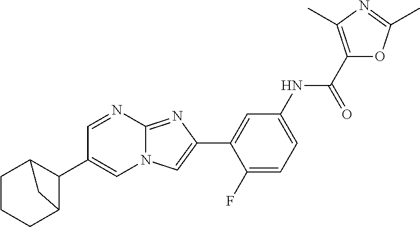 [SMILES: Cc1nc(C)c(C(=O)Nc2ccc(F)c(-c3cn4cc(C5C6CCCC5C6)cnc4n3)c2)o1] | ¹H NMR (400 MHz, CD₃OD) δ 8.83- 8.72 (m, 1H), 8.39-8.25 (m, 2H), 7.79 (dddd, J = 8.8, 6.2, 4.5, 2.3 Hz, 1H), 7.43-7.28 (m, 2H), 2.56 (d, J = 5.7 Hz, 3H), 2.48 (d, J = 1.0 Hz, 4H), 2.17 (d, J = 2.8 Hz, 1H), 1.87-1.45 (m, 8H), 1.43-1.14 (m, 1H). LCMS m/z = 446.1 (M + 1); RT = 1.09, Method 2. |
| 616 | 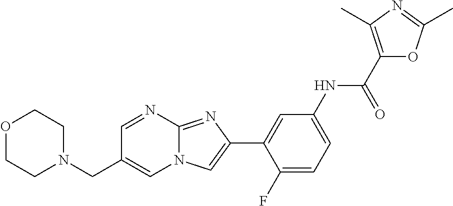 [SMILES: Cc1nc(C)c(C(=O)Nc2ccc(F)c(-c3cn4cc(CN5CCOCC5)cnc4n3)c2)o1] | ¹H NMR (400 MHz, DMSO-d₆) δ 10.34 (s, 1H), 8.93 (d, J = 2.01 Hz, 1H), 8.72 (m, 1H), 8.55 (d, J = 2.26 Hz, 1H), 8.29 (d, J = 4.02 Hz, 1H), 7.76-7.83 (m, 1H), 7.33 (m, 1H), 3.53-3.65 (m, 6H), 2.50- 2.50 (m, 1H), 2.45 (s, 4H), 2.41 (s, 3H). MS LCMS m/z = 451.1 (M + 1). LCMS m/z = 451.1 (M + 1); RT = 0.58, Method 2. |
| 617 | 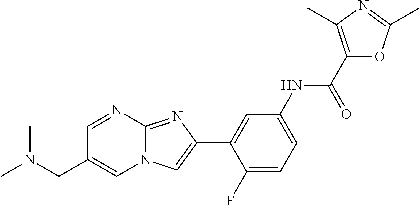 | ¹H NMR (400 MHz, DMSO-d₆) δ 10.34 (s, 1H), 8.93 (s, 1H), 8.72 (m, 1H), 8.54 (d, J = 2.26 Hz, 1H), 8.29 (d, J = 4.14 Hz, 1H), 7.74-7.86 (m, 1H), 7.27- 7.40 (m, 1H), 3.48 (s, 2H), 2.53-2.53 (m, 3H), 2.41 (s, 3H), 2.22 (s, 6H). |
| 618 | 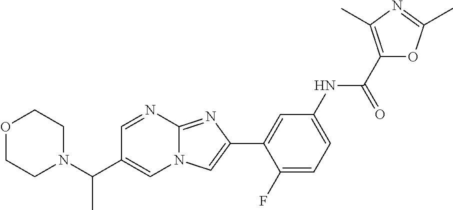 [SMILES: Cc1nc(C)c(C(=O)Nc2ccc(F)c(-c3cn4cc(C(C)N5CCOCC5)cnc4n3)c2)o1] | ¹H NMR (400 MHz, meOD) 8.80 (d, J = 1.76 Hz, 1H), 8.65 (d, J = 2.26 Hz, 1H), 8.24 (dd, J = 6.53, 2.51 Hz, 1H), 8.18 (d, J = 3.76 Hz, 1H), 7.76-7.85 (m, 1H), 7.21 (dd, J = 10.67, 9.16 Hz, 1H), 3.67-3.75 (m, 4H), 3.63 (q, J = 6.86 Hz, 1H), 2.57 (brs, 2H), 2.54 (s, 3H), 2.47-2.52 (m, 2H), 2.45 (s, 3H). LCMS m/z = 465.3 (M + 1); RT = 0.6, Method 2. |
| 619 | 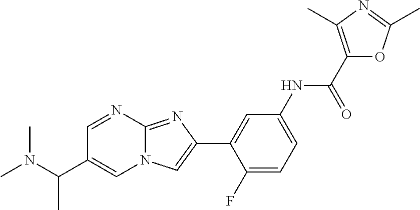 | ¹H NMR (400 MHz, DMSO-d₆) δ 10.33 (brs. 1H), 8.92 (d, J = 2.01 Hz, 1H), 8.71 (dd, J = 6.90, 2.63 Hz, 1H), 8.59 (d, J = 2.26 Hz, 1H), 8.26 (d, J = 4.27 Hz, 1H), 7.79 (dd, J = 8.16, 4.64 Hz, 1H), 7.26-7.37 (m, 1H), 3.58 (q, J = 6.69 Hz, 1H), 2.53 (s, 3H), 2.40 (s, 3H), 2.24 (s, 6H), 1.36 (d, J = 6.78 Hz, 3H). LCMS m/z = 423.1 (M + 1); RT = 0.58, Method 2. |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound of Formula A: 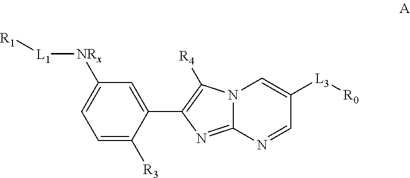
or a pharmaceutically acceptable salt, or stereoisomer thereof; wherein
Rₓ is hydrogen or C₁₋₄alkyl;
L₁ is a bond, —CH₂C(O)—, —C(O)— or —S(O)₂—;
R₁ is selected from hydrogen, nitro, C₁₋₆alkyl, C₂₋₄alkenyl, C₁₋₆alkoxy, —NR₇ₐR_{7b}, C₃₋₆cycloalkyl, C₄₋₇heterocycloalkyl, C₅₋₆heterocycloalkenyl, phenyl, benzyl and C₅₋₉heteroaryl; or —NRₓL₁R₁ is nitro; or R₁ and Rₓ with L₁ and N to which R₁ and Rₓ are respectively attached to form a C₄₋₉heterocyclyl optionally substituted by 1 or 2 oxo, wherein
R₇ₐ is hydrogen or C₁₋₄alkyl;
R_{7b} is selected from hydrogen, C₁₋₄alkyl, C₁₋₄haloalkyl, C₁₋₄alkoxy, amino, C₃₋₆cycloalkyl, C₁₋₄alkoxy substituted C₃₋₆cycloalkyl, and C₅₋₆heteroaryl, wherein the C₅₋₆heteroaryl is optionally substituted by 1 to 2 substituents independently selected from halo, C₁₋₄alky, C₁₋₄alkoxyl and amino; and wherein the available hydrogens of the C₁₋₄alkyl of R₇ₐ and R_{7b} are each optionally replaced by deuterium;
the C₁₋₆alkyl, C₂₋₄alkenyl, C₁₋₆alkoxy, amino, C₃₋₆cycloalkyl, C₄₋₇heterocycloalkyl, C₅₋₆heterocycloalkenyl, phenyl, or C₅₋₉heteroaryl of R₁ is optionally substituted by 1 to 4 substituents independently selected from hydroxy, halo, cyano, oxo, C₁₋₄alkyl, C₁₋₄hydroxyalkyl, C₁₋₄haloalkyl, C₁₋₆alkoxy, C₁₋₄haloalkoxy, C₁₋₄alkylcarbonyloxy, hydroxycarbonyl, C₁₋₄alkylcarbonyl, C₁₋₄alkoxycarbonyl, aminocarbonyl, C₁₋₄alkylsulfonyl, amino, C₁₋₄alkylamino, C₁₋₄alkylcarbonylamino, C₁₋₄alkylcarbonyl-N—C₁₋₄alkylamino, C₁₋₆alkoxycarbonylamino, C₁₋₄alkoxycarbonyl-N—C₁₋₄alkylamino, C₁₋₆alkoxy substituted C₃₋₆cycloalkyl, C₃₋₆cycloalkyl, C₄₋₆heterocycloalkyl, phenyl, and C₅₋₆heteroaryl; wherein the C₃₋₆cycloalkyl, C₄₋₆heterocycloalkyl, C₅₋₆heterocycloalkyl, phenyl or C₅₋₆heteroaryl substituent is optionally substituted by 1 to 2 substituent independently selected from halo, C₁₋₄alkyl, C₁₋₄alkylamino, or C₄₋₆heterocycloalkyl;
R₃ is selected from hydrogen, halo, cyano, C₁₋₄alkyl and C₁₋₄haloalkyl;
R₄ is selected from hydrogen, halo, C₁₋₄alkyl, C₁₋₄haloalkyl, and —C(O)R₁₀, wherein R₁₀ is selected from hydroxy, C₁₋₄alkyl, C₁₋₄alkoxy, amino, C₁₋₄alkylamino, C₃₋₆cycloalkyl and C₄₋₆heterocycloalkyl, wherein the C₁₋₄alkyl, C₁₋₄alkoxy, amino, C₁₋₄alkylamino, C₃₋₆cycloalkyl or C₄₋₆heterocycloalkyl of R₁₀ is optionally substituted by 1 to 2 substituents independently selected from hydroxyl, halo and C₁₋₄alkyl;
L₃ is selected from a bond, C₃₋₇cycloalkyl, C₄₋₇heterocycloalkyl, C₄₋₇cycloalkenyl, C₅₋₇heterocycloalkenyl, phenyl, and C₅₋₆heteroaryl;
R₀ is selected from hydrogen, hydroxy, halo, oxo, nitro, —N═CHN(CH₃)₂, C₁₋₆alkyl, C₁₋₄haloalkyl, C₂₋₄alkenyl, C₂₋₄alkynyl, C₁₋₄alkoxy, C₄₋₆heterocycloalkyloxy, —C(O)R₆, —NR₂ₐR_{2b}, —NR₅C(O)R₆, —NR₅S(O)₂R₈, S(O)₂R₈, tri-C₁₋₄alkylsilyl, C₃₋₆cycloalkyl, C₄₋₆heterocycloalkyl, C₃₋₆cycloalkenyl, C₄₋₆heterocycloalkenyl, phenyl, and C₅₋₆heteroaryl; provided when L₃ is a bond, R₀ is not hydrogen; wherein
the C₁₋₆alkyl, C₂₋₄alkenyl, C₂₋₄alkynyl, or C₁₋₄alkoxy of R₀ is optionally substituted by 1-2-substituents independently selected from hydroxy, halo, C₁₋₄alkoxy, trimethylsilylC₁₋₄alkoxy, C₁₋₄alkoxycarbonyl, —NR₁₁ₐR_{11b}, C₁₋₄alkoxycarbonylamino, C₅₋₆heterocycloalkyloxy, C₃₋₇cycloalkyl, C₅₋₆heterocycloalkyl, phenyl and C₅₋₆heteroaryl, wherein the C₃₋₇cycloalkyl, C₅₋₆heterocycloalkyl, phenyl or C₅₋₆heteroaryl substituent is optionally substituted by hydroxy, halo, C₁₋₄alkyl or C₁₋₄alkoxycarbonylamino, and wherein R₁₁ₐ and R_{11b} are independently hydrogen or C₁₋₄alkyl;
the C₄₋₆heterocycloalkyloxy, C₃₋₆cycloalkyl, C₄₋₆heterocycloalkyl, C₄₋₆heterocycloalkenyl, phenyl or C₅₋₆heteroaryl of R₀ is optionally substituted by 1 to 4 substituents independently selected from hydroxy, halo, oxo, C₁₋₄alkyl, C₁₋₄hydroxyalkyl, C₁₋₄haloalkyl, —(CH₂)₀₋₄NRₐR_{b}, C₅₋₆arylC₁₋₄alkyl, C₄₋₆heterocycloalkylC₁₋₄alkyl, C₁₋₄alkoxy, C₁₋₄alkoxycarbonyl, C₁₋₄alkylsulfonyl, C₄₋₆heterocycloalkyl, wherein Rₐ and R_{b} are each independently hydrogen, C₁₋₄alkyl or C₃₋₆cycloalkyl;
R₂ₐ is selected from hydrogen, C₁₋₄alkyl, C₁₋₄alkoxyC₁₋₄alkyl, C₁₋₄alkylcarbonyloxyC₁₋₄alkyl, and a C₁₋₄haloalkyl substituted C₅₋₆heteroaryl;
R_{2b} is selected from hydrogen, C₁₋₆alkyl, C₁₋₆haloalkyl, aminoC₁₋₄alkyl, C₁₋₄alkoxy, C₁₋₄alkoxycarbonyl, C₃₋₆cycloalkyl, C₄₋₆heterocycloalkyl, C₅₋₆heterocycloalkenyl, and C₅₋₆heteroaryl, wherein
the C₁₋₆alkyl of R_{2b} is optionally substituted by C₁₋₄alkoxy, amino, C₁₋₄alkylamino, C₄₋₆cycloalkyl, C₄₋₆heterocycloalkyl, phenyl or C₅₋₆heteroaryl, wherein the C₄₋₆cycloalkyl, C₄₋₆heterocycloalkyl, phenyl or C₅₋₆heteroaryl substituent is optionally substituted by hydroxy, halo, C₁₋₄alkyl, C₁₋₄haloalkyl, or C₁₋₄alkyoxycarbonylamino;
the C₄₋₆heterocycloalkyl, C₅₋₆heterocycloalkenyl or C₅₋₆heteroaryl of R_{2b} is optionally substituted by 1 to 2 substituents independently selected from C₁₋₄alkyl and C₁₋₄haloalkyl;
R₅ is hydrogen or C₁₋₄alkyl;
R₆ is selected from hydrogen, C₁₋₆alkyl, C₁₋₆hydroxyalkyl, C₁₋₆haloalkyl, C₂₋₆alkenyl, C₁₋₄alkoxy, C₁₋₆haloalkoxy, C₁₋₄cycloalkoxy, C₂₋₆alkenyloxy, C₃₋₆cycloalkyloxy, benzyloxy, —NR₁₂ₐR_{12b}, C₃₋₇cycloalkyl, C₄₋₇heterocycloalkyl, C₅₋₇cycloalkenyl, C₅₋₆heterocycloalkenyl, phenyl, and C₅₋₆heteroaryl, wherein
R₁₂ₐ is hydrogen or C₁₋₆alkyl,
R_{12b} is selected from hydrogen, C₁₋₆alkyl, amino and C₁₋₆alkylamino;
the C₁₋₆alkyl, C₂₋₆alkenyl, C₁₋₄alkoxy, C₃₋₆cycloalkyloxy, or amino of R₆ is optionally substituted by 1 to 2 substituents independently selected from hydroxy, halo, C₁₋₄alkyl, C₁₋₄haloalkyl, C₁₋₄alkoxy, C₁₋₄haloalkoxy, C₁₋₄alkoxyC₁₋₄alkoxy, —NR₉ₐR_{9b}, C₃₋₆cycloalkyl, C₄₋₇heterocycloalkyl, and C₅₋₆heteroaryl, wherein the C₅₋₆heterocycloalkyl or C₅₋₆heteroaryl substituent is optionally substituted by 1-2 substituents independently selected from hydroxyl, C₁₋₄alkyl and C₁₋₄alkoxycarbonyl, and wherein R₉ₐ is hydrogen or C₁₋₄alkyl and R_{9b} is selected from hydrogen, C₁₋₄alkyl, C₁₋₄alkylcarbonyl and C₁₋₄alkoxycarbonyl;
the C₃₋₆cycloalkyloxy, C₃₋₇cycloalkyl, C₄₋₇heterocycloalkyl, C₅₋₇cycloalkenyl, or C₅₋₆heterocycloalkenyl of R₆ is optionally substituted by 1 to 3 substituents independently selected from hydroxy, halo, cyano, oxo, C₁₋₄alkyl, haloC₁₋₄alkyl, C₁₋₄alkoxylC₁₋₄alkyl, C₁₋₄alkoxycarbonylaminoC₁₋₄alkyl, C₁₋₄alkoxyl, C₁₋₄alkoxycarbonyl, amino, aminocarbonyl, and
the phenyl or C₅₋₆heteroaryl of R₆ is optionally substituted with 1-2 substituents independently selected from hydroxy, halo, cyano, C₁₋₄alkyl, C₁₋₄hydroxyalkyl, C₁₋₄haloalkyl, C₁₋₄alkoxy, C₁₋₄alkoxycarbonyl, —(CH₂)₀₋₄NR₁₃ₐR_{13b}, and C₁₋₄alkoxycarbonylamino, wherein the C₁₋₄alkoxy substituent is optionally substituted by C₁₋₄alkylC₅₋₆heterocycloalkyl, and wherein R₁₃ₐ and R_{13b} are each independently hydrogen or C₁₋₄alkyl; and
R₈ is C₁₋₄alkyl or C₁₋₄alkylamino.

2. The compound according to claim 1, wherein
Rₓ is hydrogen;
L₁ is a bond or —C(O)—;
R₁ is selected from C₁₋₆alkoxy, C₁₋₆alkoxyC₁₋₆alkoxy, NR₇ₐR_{7b}, C₃₋₆cycloalkyl, C₄₋₇heterocycloalkyl, C₅₋₆heterocycloalkenyl, phenyl, benzyl, and C₅₋₉heteroaryl, wherein
R₇ₐ is hydrogen or C₁₋₄alkyl;
R_{7b} is selected from hydrogen, C₁₋₄alkyl, C₁₋₄haloalkyl, C₁₋₄alkoxy, amino, C₁₋₄alkoxy substituted C₃₋₆cycloalkyl, C₃₋₆cycloalkyl, and C₅₋₆heteroaryl, wherein the C₅₋₆heteroaryl is optionally substituted by 1 to 2 substituents independently selected from halo and C₁₋₄alkyl;
the C₃₋₆cycloalkyl, C₄₋₇heterocycloalkyl, C₅₋₆heterocycloalkenyl, phenyl, or C₅₋₉heteroary of R₁ is optionally substituted by 1-2 substituents independently selected from hydroxy, halo, cyano, oxo, C₁₋₄alkyl, C₁₋₄haloalkyl, C₁₋₄alkoxy, amino, C₁₋₄alkylamino, C₁₋₄alkylcarbonyl, am inocarbonyl, C₁₋₄alkylsulfonyl, C₃₋₆cycloalkyl, and
C₄₋₆heterocycloalkyl;
R₃ is hydrogen or halo;
R₄ is hydrogen or halo;
L₃ is a selected from a bond, C₃₋₇cycloalkyl, C₄₋₇heterocycloalkyl, C₅₋₆cycloalkenyl, C₅₋₆heterocycloalkenyl, phenyl, and C₅₋₆heteroaryl;
R₀ is selected from hydrogen, halo, oxo, C₁₋₆alkyl, C₁₋₄haloalkyl, C₂₋₄alkenyl, C₂₋₄alkynyl, C₁₋₄alkoxy, C₄₋₆heterocycloalkyloxy, —C(O)R₆, —NR₂ₐR_{2b}, —NR₅C(O)R₆, -tri-C₁₋₄alkylsilyl, C₄₋₆heterocycloalkyl, C₄₋₆heterocycloalkenyl, phenyl, and C₅₋₆heteroaryl; provided when L₃ is a bond, R₀ is not hydrogen; wherein
the C₁₋₆alkyl, C₂₋₄alkynyl or C₁₋₄alkoxy of R₀ is optionally substituted by 1 to 2 substituents independently selected from hydroxy, C₁₋₄alkoxy, trimethylsilylC₁₋₄alkoxy, C₁₋₄alkoxycarbonyl, —NR₁₁ₐR_{11b}, C₁₋₄alkoxycarbonylamino, C₃₋₇cycloalkyl, C₅₋₆heterocycloalkyl, phenyl and C₅₋₆heteroaryl; wherein the C₃₋₇cycloalkyl or C₅₋₆heterocycloalkyl substituent is optionally substituted by hydroxy, C₁₋₄alkyl or C₁₋₄alkoxycarbonylamino, and wherein R₁₁ₐ and R_{11b} are independently hydrogen or C₁₋₄alkyl;
the C₄₋₆heterocycloalkyloxy, C₄₋₆heterocycloalkyl, C₄₋₆heterocycloalkenyl, phenyl or C₅₋₆heteroaryl of R₀ is optionally substituted by 1 to 4 substituents independently selected from hydroxy, halo, C₁₋₄alkyl, C₁₋₄haloalkyl, —(CH₂)₀₋₄NRₐR_{b}, C₅₋₆arylC₁₋₄alkyl, C₄₋₆heterocycloalkylC₁₋₄alkyl, C₁₋₄alkoxy, C₁₋₄alkoxycarbonyl, C₁₋₄alkylsulfonyl, and C₄₋₆heterocycloalkyl, wherein Rₐ and R_{b} are independently hydrogen or C₁₋₄alkyl;
R₂ₐ is selected from hydrogen, C₁₋₄alkyl, C₁₋₄alkoxyC₁₋₄alkyl, C₁₋₄alkylcarbonyloxyC₁₋₄alkyl, and a C₁₋₄haloalkyl substituted C₅₋₆heteroaryl;
R_{2b} is selected from hydrogen, C₁₋₄alkyl, C₁₋₄haloalkyl, aminoC₁₋₄alkyl, C₁₋₄alkoxy, C₁₋₄alkoxycarbonyl, C₃₋₆cycloalkyl, C₄₋₆heterocycloalkyl, C₅₋₆heterocycloalkenyl, and C₅₋₆heteroaryl, wherein
the C₁₋₄alkyl of R_{2b} is optionally substituted by C₁₋₄alkoxy, C₁₋₄alkylamino, C₄₋₆cycloalkyl, or C₅₋₆heteroaryl, wherein the C₄₋₆cycloalkyl substituent is optionally substituted by C₁₋₄alkyoxycarbonylamino; and
the C₄₋₆heterocycloalkyl, C₅₋₆heterocycloalkenyl or C₅₋₆heteroaryl of R_{2b} is optionally substituted by 1 to 2 substituents independently selected from C₁₋₄alkyl and C₁₋₄haloalkyl;
R₅ is hydrogen or C₁₋₄alkyl;
R₆ is selected from hydrogen, C₁₋₆alkyl, C₁₋₆hydroxyalkyl, C₁₋₄haloalkyl, C₁₋₄alkoxy, C₂₋₄alkenyloxy, C₃₋₆cycloalkyloxy, benzyloxy, —NR₁₂ₐR_{12b}, C₃₋₇cycloalkyl, C₄₋₇heterocycloalkyl, C₅₋₇cycloalkenyl, C₅₋₆heterocycloalkenyl, phenyl, and C₅₋₆heteroaryl, wherein
R₁₂ₐ is hydrogen or C₁₋₆alkyl;
R_{12b} is selected from hydrogen, C₁₋₆alkyl, amino and C₁₋₆alkylamino;
the C₁₋₄alkyl or C₁₋₄alkoxy of R₆ is optionally substituted by 1 to 2 substituents independently selected from halo, hydroxy, C₁₋₄haloalkyl, C₁₋₄alkoxy, C₁₋₄haloalkoxy, C₁₋₄alkoxyC₁₋₄alkoxy, —NR₉ₐR_{9b}, and C₄₋₇heterocycloalkyl, wherein R₉ₐ is hydrogen or C₁₋₄alkyl and R_{9b} is selected from hydrogen, C₁₋₄alkyl, and C₁₋₄alkoxycarbonyl;
the C₃₋₆cycloalkyloxy, C₃₋₇cycloalkyl, C₄₋₇heterocycloalkyl, C₅₋₇cycloalkenyl, or C₅₋₆heterocycloalkenyl of R₆ is optionally substituted by 1 to 3 substituents independently selected from hydroxy, halo, cyano, oxo, C₁₋₆alkyl, C₁₋₄alkoxylC₁₋₄alkyl, C₁₋₄alkoxy, C₁₋₄alkoxycarbonyl, and amino;
the phenyl or C₅₋₆heteroaryl of R₆ is optionally substituted with 1 to 2 substituents independently selected from halo, cyano, C₁₋₄alkyl, C₁₋₄hydroxyalkyl, C₁₋₄haloalkyl, C₁₋₄alkoxy, C₁₋₄alkoxycarbonyl, —(CH₂)₀₋₄NR₁₃ₐR_{13b}, and C₁₋₄alkoxycarbonylamino, wherein the C₁₋₄alkoxy substituent is optionally substituted by C₁₋₄alkylC₅₋₆heterocycloalkyl, and wherein R₁₃ₐ and R_{13b} are independently hydrogen or C₁₋₄alkyl; and
R₈ is C₁₋₄alkyl.

3. The compound according to claim 1, wherein R₁ is selected from C₁₋₆alkoxy, C₃₋₆cycloalkyl, C₄₋₇heterocycloalkyl, C₅₋₆heterocycloalkenyl, phenyl, benzyl, and C₅₋₉heteroaryl, wherein the C₃₋₆cycloalkyl, C₄₋₇heterocycloalkyl, C₅₋₆heterocycloalkenyl, phenyl, or C₅₋₉heteroaryl is optional substituted by 1 to 2 substituents independently selected from hydroxy, halo, cyano, oxo, C₁₋₄alkyl, C₁₋₄haloalkyl, C₁₋₄alkoxy, amino, C₁₋₄alkylamino, C₁₋₄alkylcarbonyl, aminocarbonyl, C₁₋₄alkylsulfonyl, C₃₋₆cycloalkyl, and C₄₋₆heterocycloalkyl.

4. The compound according to claim 1, wherein R₁ is selected from methoxy, ethoxy, isopropyloxy, cyclopropyl, cyclobutyl, cyclopentyl, cyclohexyl, azetidinyl, pyrrolidinyl, piperidinyl, piperazinyl, morpholinyl, tetrahydropyranyl, dioxanyl, 5-azaspiro[2,4]heptanyl, 2-oxa-6-aza-spiro[3,3]heptanyl, oxaazobicyclo[2.2.1]heptanyl, dihydrooxazolyl, phenyl, benzyl, pyrrolyl, pyrazolyl, imidazolyl, furanyl, isooxazolyl, oxazolyl, thiazolyl, pyridinyl, pyrimidinyl, indolyl, furo[2,3-c]pyridinyl, and imidazo[1,2-a]pyrimidinyl, wherein the above C₃₋₆cycloalkyl, C₄₋₇heterocycloalkyl, C₅₋₆heterocycloalkenyl, phenyl, or C₅₋₉heteroaryl is optional substituted by 1 to 2 substituents independently selected from hydroxy, halo, cyano, oxo, methyl, trifluoromethyl, methoxy, amino, —NHCH₃, methylcarbonyl, aminocarbonyl, methylsulfonyl, cyclopropyl, and morpholinyl.

5. The compound according to claim 1, wherein R₀ is selected from hydrogen, halo, oxo, C₁₋₆alkyl, C₁₋₄hydroxyalkyl, C₁₋₄haloalkyl, C₂₋₄alkenyl, trimethylsilylC₁₋₆alkyloxyC₁₋₆alkyl, —NR₂ₐR_{2b}, —NHC(O)R₆, C₄₋₆heterocycloalkyl, C₄₋₆heterocycloalkenyl, phenyl and C₅₋₆heteroaryl, wherein
the C₄₋₆heterocycloalkyl, C₄₋₆heterocycloalkenyl, phenyl or C₅₋₆heteroaryl is optionally substituted by 1 to 3 substituents independently selected from hydroxy, halo and C₁₋₄alky, C₁₋₄haloalkyl, and C₁₋₄alkoxycarbonyl;
R₂ₐ is selected from hydrogen, C₁₋₄alkyl, C₁₋₆alkoxyC₁₋₄alkyl, C₁₋₄alkylcarbonyloxyC₁₋₄alkyl, and a C₁₋₄haloalkyl substituted C₅₋₆heteroaryl;
R_{2b} is hydrogen, C₁₋₄alkyl, C₁₋₄alkoxy, or C₁₋₄alkoxycarbonyl;
R₆ is selected from C₁₋₄alkoxy, C₃₋₇cycloalkyl, C₄₋₇heterocycloalkyl, C₅₋₇cycloalkenyl, C₅₋₆heterocycloalkenyl, phenyl and C₅₋₆heteroaryl, wherein the C₃₋₇cycloalkyl, C₄₋₇heterocycloalkyl, C₅₋₇cycloalkenyl, or C₅₋₆heterocycloalkenyl is optionally substituted by 1 to 2 substituents independently selected from hydroxy, halo, cyano, oxo, C₁₋₆alkyl, C₁₋₄alkoxylC₁₋₄alkyl, C₁₋₄alkoxy, C₁₋₄alkoxycarbonyl, and amino, and wherein
the phenyl or C₅₋₆heteroaryl is optionally substituted by 1 to 2 substituents independently selected from halo, cyano, C₁₋₆alkyl, C₁₋₄hydroxyalkyl, C₁₋₄haloalkyl, C₁₋₄alkoxycarbonyl, amino, C₁₋₄alkyamino, and C₁₋₄alkoxycarbonylamino.

6. The compound according to claim 1, wherein R₀ is selected from hydrogen, halo, oxo, methyl, ethyl, isopropyl, isobutyl, —CH═CH₂, —(CH₂)₂OH, —CHF₂, —CF₃, —CH₂CF₃, —(CH₂)₂CF₃, trimethylsilylethoxymethyl, —NHCH₃, —NH— trifluoromethylpyridinyl, —N(CH₂OC(O)CH₂CH₃)(C(O)OCH(CH₃)₂), —NHC(O)R₆, azetidinyl, piperidinyl, piperazinyl, morpholinyl, dihydro-1,4-dioxinyl, dihydro-2H-pyranyl, phenyl, imidazolyl, oxazolyl, oxadiazolyl, thiazolyl, and pyridinyl, wherein the azetidinyl, piperidinyl, piperazinyl, morpholinyl, dihydro-1,4-dioxinyl, dihydro-2H-pyranyl, phenyl, imidazolyl, oxazolyl, oxadiazoyl, thiazolyl, or pyridinyl is optionally substituted by 1 to 3 substituents independently selected from hydroxy, halo, C₁₋₄alkyl, C₁₋₄haloalkyl, and —C(O)OCH₃, wherein
R₆ is selected from —OCH₃, —OCH(CH₃)₂, cyclopropyl, cyclobutyl, cyclohexyl, azetidinyl, pyrrolidinyl, piperidinyl, piperazinyl, oxetanyl, tetrahydrofuranyl, tetrahydropyranyl, morpholinyl, dioxanyl, oxazepanyl, oxabicyclo[2.2.1]heptanyl, bicyclo[2.2.1]hept-2-ene, dihydro-1,4-dioxinyl, phenyl, pyridinyl, imidazolyl, and triazolyl, wherein
the cyclopropyl, cyclobutyl, cyclohexyl, azetidinyl, pyrrolidinyl, piperidinyl, piperazinyl, oxetanyl, tetrahydrofuranyl, tetrahydropyranyl, morpholinyl, dioxanyl, oxazepanyl, oxabicyclo[2.2.1]heptanyl, bicyclo[2.2.1]hept-2-ene, or dihydro-1,4-dioxinyl of R₆ is optionally substituted by 1 to 3 substituents independently selected from hydroxy, halo, cyano, oxo, methyl, methoxymethyl, methoxy, butoxycarbonyl, amino, and
the phenyl, pyridinyl, imidazolyl, or triazolyl of R₆ is optionally substituted by halo, cyano, methyl, ethyl, isopropyl, —(CH₂)₂OH, —CHF₂, —CF₃, —CH₂CF₃, —(CH₂)₂CF₃, amino, —NHCH₃, —N(CH₃)₂, and butoxycarbonylamino.

7. The compound according to claim 1, wherein the compound is of Formula A1: 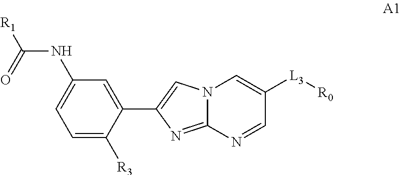
or a pharmaceutically acceptable salt, or stereoisomer thereof; wherein
R₁ is C₄₋₇heterocycloalkyl or C₅₋₆heteroaryl, each of which is optionally substituted by 1-2 substituents independently selected from hydroxy, halo, cyano, oxo, C₁₋₄alkyl, C₁₋₄haloalkyl, C₁₋₆alkoxy, C₁₋₄alkylcarbonyl, am inocarbonyl, C₁₋₄alkylsulfonyl, amino, —NHCH₃, C₃₋₆cycloalkyl, and C₄₋₆heterocycloalkyl;
R₃ is hydrogen or halo;
L₃ is selected from a bond, C₃₋₇cycloalkyl, C₄₋₆heterocycloalkyl, C₅₋₆cycloalkenyl, C₅₋₆heterocycloalkenyl, phenyl, and C₅₋₆heteroaryl;
R₀ is selected from hydrogen, halo, C₁₋₆alkyl, C₁₋₄hydroxyalkyl, C₁₋₄haloalkyl, NR₂ₐR_{2b}, phenyl, and C₅₋₆heteroaryl; provided when L₃ is a bond, R₀ is not hydrogen; wherein
the phenyl or C₅₋₆heteroaryl of R₀ is optionally substituted by 1 to 2 substituents independently selected from halo, C₁₋₄alky, C₁₋₄alkyamino, and C₁₋₄alkoxy, and
R₂ₐ and R_{2b} are each independently hydrogen or C₁₋₄alkyl.

8. The compound according to claim 7, wherein R₁ is selected from azetidinyl, pyrrolidinyl, piperidinyl, piperazinyl, morpholinyl, tetrahydropyranyl, dioxanyl, pyrrolyl, pyrazolyl, imidazolyl, furanyl, isooxazolyl, oxazolyl, thiazolyl, pyridinyl, and pyrimidinyl, each of which is optional substituted by 1 to 2 substituents independently selected from hydroxy, halo, cyano, oxo, methyl, trifluoromethyl, methoxy, amino, and —NHCH₃.

9. The compound according to claim 7, wherein R₁ is selected from: 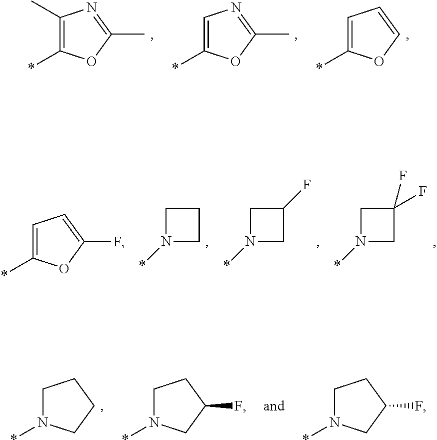
wherein “*” indicates the point of attachment of R₁.

10. The compound according to any one of claims 1 to 9, wherein L₃ is selected from C₃₋₇cycloalkyl, C₄₋₆heterocycloalkyl, C₅₋₆cycloalkenyl, C₅₋₆heterocycloalkenyl, phenyl, and C₃₋₆heteroaryl.

11. The compound according to claim 7, wherein L₃ is selected from (note, no bond) cyclopropyl, cyclobutyl, cyclopentyl, bicyclo[3.1.1]heptanyl, pyrrolidinyl, piperidinyl, tetrahydropyranyl, morpholinyl, cyclopentenyl, tetrahydropyridinyl, dihydrofuranyl, dihydropyranyl, phenyl, pyrrolyl, pyrazolyl, imidazolyl, triazolyl, oxazolyl, isooxazolyl, pyridinyl, pyridazinyl, pyrimidinyl, and pyrazinyl.

12. The compound according to any one of claims 1 to 9, wherein L₃ is selected from cyclopropyl, cyclobutyl, tetrahydropyranyl, cyclopentenyl, dihydrofuranyl, dihydropyranyl, phenyl, pyrazolyl, imidazolyl, thiazolyl, pyridinyl, and pyrimidinyl.

13. The compound according to claim 7, wherein R₀ is selected from hydrogen, halo, methyl, ethyl, isopropyl, isobutyl, —(CH₂)₂OH, —CHF₂, —CF₃, —CH₂CF₃, —(CH₂)₂CF₃, —NHCH₃, phenyl, and pyridinyl, wherein the phenyl or pyridinyl is optionally substituted by 1-2 substituents independently selected from halo and methyl.

14. The compound according to claim 7, wherein -L₃-R₀ is selected from methyl, ethyl, isopropyl, —CHF₂, —CF₃, —CH₂CF₃, —(CH₂)₂CF₃, 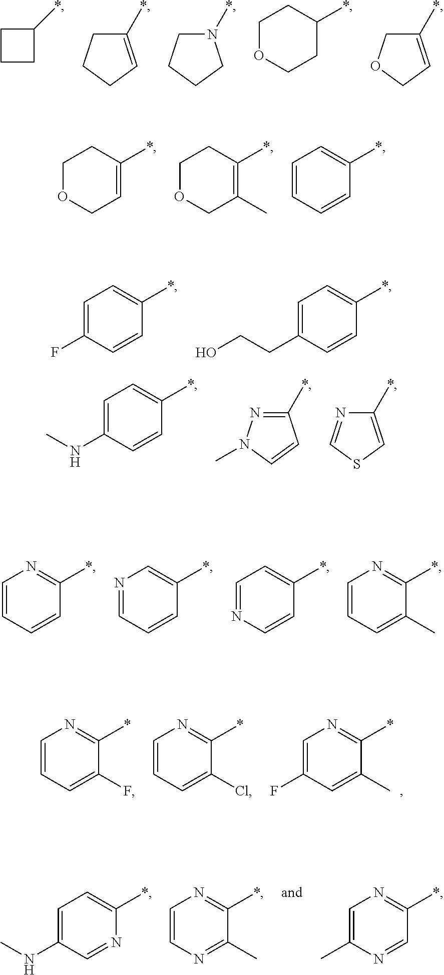
wherein “*” represents the point of attachment of -L₃R₀.

15. The compound according to claim 1, wherein the compound is of Formula I: 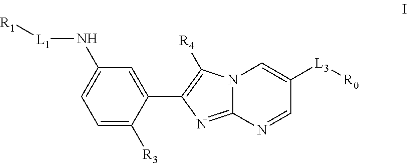
or a pharmaceutically acceptable salt, or stereoisomer thereof; wherein
L₁ is —C(O)— or —S(O)₂—;
R₁ is selected from hydrogen, nitro, C₁₋₄alkyl, C₁₋₆alkoxy, amino, C₅₋₉heteroaryl, C₃₋₆cycloalkyl and C₄₋₆heterocycloalkyl, each of which is optionally substituted by 1-2 substituent independently selected from halo, cyano, amino, C₁₋₄alkyl, haloC₁₋₄alkyl, C₁₋₆alkoxy, and C₁₋₄alkylcarbonyl; or —NHL₁R₁ is nitro;
R₃ is selected from hydrogen, halo, cyano, C₁₋₄alkyl and haloC₁₋₄alkyl;
R₄ is selected from hydrogen, C₁₋₄alkyl, haloC₁₋₄alkyl, and —C(O)R₁₀, wherein R₁₀ is hydroxy, C₁₋₄alkyl, C₁₋₄alkoxy, amino, C₁₋₄alkylamino, C₃₋₆cycloalkyl and C₄₋₆heterocycloalkyl, each of which is independently optionally substituted by 1-2 substituents independently selected from hydroxyl, halo and C₁₋₄alkyl;
L₃ is a bond, phenylene, or C₅₋₆heteroarylene;
R₀ is selected from hydrogen, hydroxyl, halo, nitro, —N═CHN(CH₃)₂, C₁₋₄alkyl, C₁₋₄alkoxy, —NR₂ₐR_{2b}, —NR₅C(O)R₆, —NR₅S(O)₂R₈, C₃₋₆cycloalkyl, C₄₋₆heterocycloalkyl, C₄₋₆heterocycloalkenyl, phenyl and C₅₋₆heteroaryl; wherein
the C₁₋₄alkyl or C₁₋₄alkoxy is optionally substituted by 1-2 substituents independently selected from C₁₋₄alkoxy, amino, phenyl and C₅₋₆heteroaryl; wherein the phenyl or C₅₋₆heteroaryl is optionally substituted by halo or C₁₋₄alkyl;
the C₃₋₆cycloalkyl, C₄₋₆heterocycloalkyl, C₄₋₆heterocycloalkenyl, phenyl and C₅₋₆heteroaryl of R₀ is optionally substituted with halo, oxo, C₁₋₄alkyl, hydroxyC₁₋₄alkyl, haloC₁₋₄alkyl, C₁₋₄alkoxy, amino, C₁₋₄alkylamino, and —(CH₂)₁₋₄NRₐR_{b}, wherein Rₐ and R_{b} are each independently hydrogen, C₁₋₄alkyl or C₃₋₆cycloalkyl;
R₂ₐ is hydrogen or C₁₋₄alkyl;
R_{2b} is selected from hydrogen, C₁₋₄alkyl, wherein the alkyl is optionally substituted by amino, C₄₋₆heterocycloalkyl, phenyl or C₅₋₆heteroaryl, wherein the C₄₋₆heterocycloalkyl, phenyl or C₅₋₆heteroaryl is optionally substituted by hydroxyl, halo or C₁₋₄alkyl;
R₅ is hydrogen or C₁₋₄alkyl;
R₆ is selected from hydrogen, C₁₋₆alkyl, C₁₋₄alkoxy, C₃₋₆cycloalkoxy, amino, C₃₋₆cycloalkyl, C₅₋₆heterocycloalkyl, and C₅₋₆heteroaryl, wherein
the C₁₋₄alkyl, C₁₋₄alkoxy, C₃₋₆cycloalkoxy, and amino of R₆ are each optionally substituted by 1-2 substituents independently selected from halo, hydroxy, C₁₋₄alkyl, haloC₁₋₄alkyl, C₁₋₄alkoxy, —NR₉ₐR_{9b}, C₃₋₆cycloalkyl, C₅₋₆heterocycloalkyl, and C₅₋₆heteroaryl, wherein R₉ₐ is hydrogen or C₁₋₄alkyl and R_{9b} is selected from hydrogen, C₁₋₄alkyl, C₁₋₄alkylcarbonyl and C₁₋₄alkoxycarbonyl, and the C₅₋₆heterocycloalkyl and C₅₋₆heteroaryl are each optionally substituted by 1-2 substituents independently selected from hydroxyl, C₁₋₄alkyl and C₁₋₄alkoxycarbonyl,
the C₅₋₆heteroaryl of R₆ is optionally substituted with 1-2 substituents selected from hydroxy, C₁₋₄alkyl, and C₁₋₄alkoxycarbonyl,
the C₃₋₆cycloalkyl or C₄₋₆heterocycloalkyl of R₆ are each independently optionally substituted by 1-2 substituents independently selected from halo, cyano, hydroxy, C₁₋₄alkyl, haloC₁₋₄alkyl, C₁₋₄alkoxylC₁₋₄alkyl, aminocarbonyl, C₁₋₄alkoxycarbonyl, and C₁₋₄alkoxycarbonylaminoC₁₋₄alkyl, and
R₈ is C₁₋₄alkyl or C₁₋₄alkylamino.

16. The compound according to claim 15, wherein -L₁-R₁ is selected from

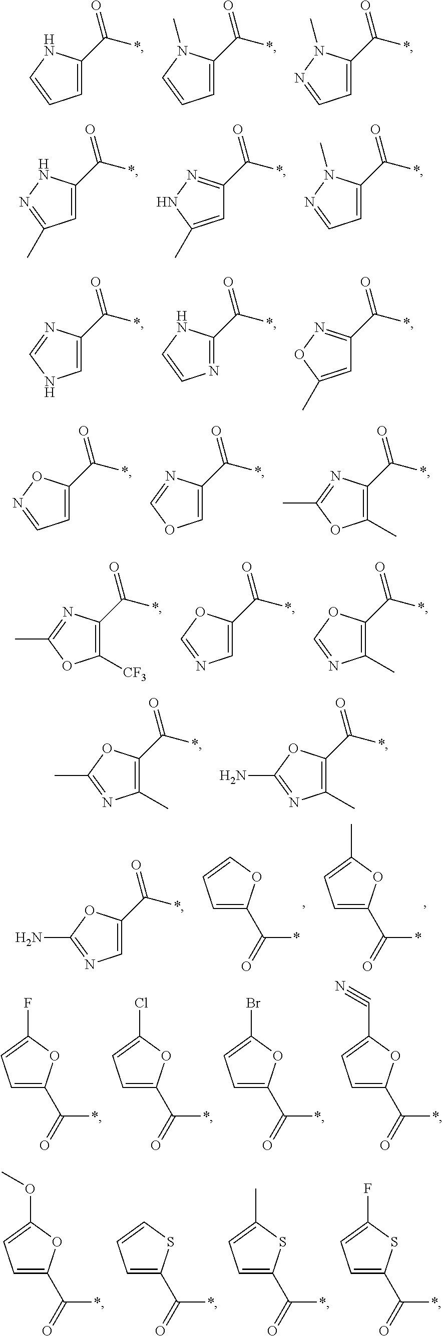

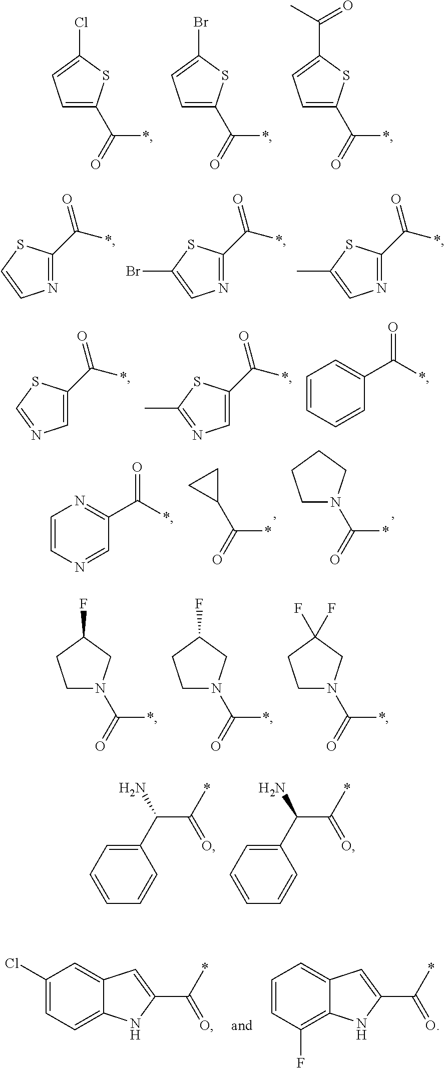

17. The compound according to claim 15, wherein R₃ is chloro or fluoro,

18. The compound according to claim 15, wherein R₄ is hydrogen.

19. The compound according to claim 15, wherein R₀ is selected from hydrogen, halo, nitro, hydroxyl, C₁₋₄alkoxy, amino, C₁₋₄alkylamino, —NH(CH₂)₁₋₂-phenyl, —NR₅C(O)R₆, —NR₅S(O)₂R₈, oxazolidin-2-one, 1,2,4-triazol-5(4H)-one, pyrrolidin-2-one, phenyl and C₅₋₆heteroaryl; wherein
the oxazolidin-2-one, 1,2,4-triazol-5(4H)-one, pyrrolidin-2-one, phenyl or C₅₋₆heteroaryl is optionally substituted with halo, C₁₋₄alkyl, C₁₋₄alkoxy, amino, C₁₋₄alkylamino, -(CH₂)₁₋₄OH, and -(CH₂)₁₋₄NRₐR_{b}, wherein Rₐ and R_{b} are each independently hydrogen, C₁₋₄alkyl or C₃₋₆cycloalkyl;
R₅ is hydrogen or C₁₋₄alkyl;
R₆ is selected from C₁₋₆alkyl, C₁₋₆alkoxyl, C₃₋₆cycloalkyl, C₅₋₆heterocycloalkyl, and C₅₋₆heteroaryl, each of which is optionally substituted with 1 to 2 substituents independently selected from hydroxyl, C₁₋₄alkoxy, amino, C₁₋₄alkylamino; and
R₈ is C₁₋₄alkyl or C₁₋₄alkylamino.

20. The compound according to claim 1 selected from the compounds listed in Table II (pages 232 to 354), wherein the corresponding names of said compounds are listed on pages 45 to 70.

21. A pharmaceutical composition comprising a compound according to claim 1 as an active ingredient and at least one excipient.

22. A method for treating, preventing, inhibiting, ameliorating, or eradicating the pathology and/or symptomology of a disease caused by a parasite, comprising administering to a subject a therapeutically effective amount of a compound according to claim 1, wherein the disease is selected from Leishmaniasis, Human African Trypanosomiasis and Chagas disease, and wherein the administering is optionally in combination with a second agent.
